(19) United States
(12) Patent Application Publication
(10) Pub. No.: US 2011/0117134 A1
(43) Pub. Date: May 19, 2011

(54) Gene library of Brucella suis surface antigens

(76) Inventors: Xuan Z. Ding, Rockville, MD (US); David I. Hoover, Rockville, MD (US); Ian T. Paulsen, Rockville, MD (US)

(21) Appl. No.: 12/590,914

(22) Filed: Nov. 16, 2009

(51) Int. Cl. A61K 39/10; C40B 40/10; C40B 40/06; C40B 30/04

(52) U.S. Cl. 424/252.1; 506/18; 506/16; 506/9

(57) ABSTRACT

The invention relates to a bacterial antigen library of Brucella suis surface antigens, its preparation and use. The library comprises 205 surface protein genes of Brucella suis constructed in an expression vector. The library can be used to prepare recombinant Brucella surface antigens. The expressed proteins derived from the gene library are important for the development of new vaccines to prevent brucellosis, or diagnostic kits to detect Brucellosis. The proteins prepared from the gene library can also be used to prepare a protein array to investigate pathogenic mechanism of brucellosis.

### GOVERNMENT INTEREST

The invention described herein may be manufactured, used and licensed by or for the U.S. Government.

### BACKGROUND OF THE INVENTION

1. Field of the Invention

The invention relates to a bacterial antigen library, its preparation and use. The library comprises 205 surface protein genes of Brucella suis constructed in an expression vector.

2. Brief Description of Related Art

Brucella, an aerobic, non-spore forming, non-motile Gram-Negative coccobacillus. Brucellosis typically occurs in humans after ingestion of unpasteurized dairy products or after occupational contact with infected humans.

Medical defense against the bio-terror threat posed by Brucella would be strengthened by development of a human vaccine and improved diagnostic tests. Central to advancement of these goals is discovery of bacterial constituents that are immunogenic or antigenic for humans.

Until now, there has not been any research group or person that has published or developed recombinant constructs that may be directly used to express all 205 surface proteins of B. suis.

The present inventors have developed a library of a set of putative Brucella surface antigens that can be used by researchers to develop new human vaccines to prevent brucellosis, or diagnostic tests or kits to detect Brucellosis. The proteins can be used to prepare a protein array to investigate pathogenic mechanism of brucellosis.

The complete library of Brucella surface antigens was prepared in a 96 well format and used for an antigen array to screen infectious specimens such as serum and blood cells for the presence of Brucella. The geometric format of the surface antigen array provides a direct technical platform to develop vaccines, methods and drugs to protect people from infectious disease.

The recombinant cells are important and valuable for the following reasons. They allow expression of a bacterial antigen library that is different from any other known library and provide definite genes and proteins used for screening new recombinant vaccines and drugs. This is also a novel technical platform to develop new diagnostic agents and vaccine candidates. This library contains a large set of surface antigens of Brucella for specific diagnosis of infection. The, recombinant proteins provided from this library include purification handles of 6-His and V5 that permit arrangement in an arrayed format or adherence to solid supports that promote their usefulness for other large-scale preparative and screening projects. The array design lends itself to further analysis using bioinformatics approaches. The library will be a valuable resource. These plates can be directly provided to scientists of universities, institutes, and companies for their screening and array studies. Any individual, specific antigen can also be prepared on a large scale from the recombinant library and purified using convenient 6-His and V5 purification handles expressed on each protein.

Therefore, the present invention provides a gene library of putative Brucella suis surface antigens.

The present invention also provides a method of preparation of a gene library of Brucella suis surface antigens.

The present invention permits the cloning and expression of a set of 205 genes of Brucella suis surface protein into expression vectors.

The present invention provides a set of recombinant constructs of Brucella suis surface antigens in 96 well format for further serum and cellular screening.

The present invention provides a method of preparing a vaccine using the gene library of Brucella suis surface antigens.

The present invention provides antigens for diagnosis and prophylaxis against brucellosis.

These features and others will become apparent upon reading the detailed description.

### SUMMARY OF THE INVENTION

The invention relates to a complete bacterial antigen library of Brucella suis surface antigens, its preparation and use. The library comprises 205 surface protein genes of Brucella suis constructed in an expression vector. The library can be used to prepare recombinant Brucella surface antigens. The expressed proteins derived from the gene library are important for the development of new vaccines to prevent brucellosis, or diagnostic kits to detect brucellosis. The proteins prepared from the gene library can also be used to prepare a protein array to investigate pathogenetic mechanisms of brucellosis.

### BRIEF DESCRIPTION OF THE FIGURES

FIG. 1 is a digital image of gel confirmation of B. suis SP Genes synthesized by PCR; and

FIG. 2 is a digital image of PCR identification of plasmids using primers located at T7 priming sites.

### DETAILED DESCRIPTION

From the genomic information of the complete sequences of B. suis, 205 open reading frames that encode surface antigens of Brucella were defined and selected. These putative Brucella surface antigens were identified as surface antigens by running prediction algorithms. These antigen genes were PCR synthesized and then constructed into an expression vector, pET101/D-TOPO (Invitrogen Life Technologies, California). The recombinant constructs were transferred into E. coli TOP10 cells (Invitrogen Life Technologies, California) for amplification. The amplified pET-B-SAs encoding surface antigens were collected and purified from the cells. Recombinant plasmids were confirmed to contain surface antigen sequence using PCR method. The positive recombinant E. coli cells are stored at −80° C. in two 96 deep well plates containing LB media with 15% glycerol and 100 ug/ml ampicillin.

### MATERIALS AND METHODS

We report the results of the use of a Highly Efficient Cloning and Expression System (herein after referred to as HECES) developed by the inventors for attempted expression of a set of 205 B. suis ORFs predicted to encode SPs (surface or secreted proteins). We find that this application of the HECES, which employs methods designed to minimize adjustment of production conditions for individual proteins, results in expression of 71% to 91% of the ORFs, depending on the specificity of the criteria used to demonstrate the presence of protein. Proteins were prepared at high purity in an assay-friendly, microwell, fixed-support format.

The following is a flow chart of the method of HECES.

### Flow Chart of HECES:

PCR synthesis of genes (Storage)
   ↓
E-gel detection of PCR products
   ↓
Ligation of genes into pET vector
   ↓
Transformation of TOP10 cells
   ↓
Spread agar plates and culture
   ↓
Pick 2 colonies per gene and culture
   ↓
Turbo miniprep of plasmids
   ↓
E-gel detection of plasmids
   ↓
PCR verification of plasmids
   ↓
E-gel detection of PCR products
   ↓
Preparation of Recombinant Plasmids

Preparation of Glycerol Stock of Each Recombinant Cells.

The HECES allows expression of large numbers of bacterial proteins based on genomic sequence information. Purified proteins are produced with high efficiency in a microarray format conducive to analysis of their sero-reactivity against serum from immunized animals. This method is applicable at either small or large scale of protein processing. In this study, we used the Invitrogen directional pET 101/D-TOPO cloning system instead of the Gateway cloning system, but other methods are as previously described.

Prediction of B. suis surface proteins (SPs): A bioinformatic screen of the complete B. suis predicted proteome was undertaken to identify putative surface exposed or secreted B. suis proteins. The bioinformatic pipeline ran a series of searches and analyses including prediction of type I and type II signal peptidase cleavage sites using SignalP, HMM and Blast searches for homology with known outer membrane proteins or protein families, transmembrane segment (TMS) prediction using TMHMM, motif searches for choline, fibrinogen and integrin binding domains, and the identification of repetitive amino acid domains. These data were collated and a set of 205 putative SPs was selected for protein expression. Proteins were included in the set if they had fewer than 3 predicted TMSs and one or more other characteristics supporting their prediction as SPs. Primer design and subsequent gene cloning and protein expression were conducted based on the bioinformatic screen predictions. Subsequently, complete annotation of the genome [5] resulted in re-assignment of the locus tags or alternative start or stop positions for some of the predicted genes. Predicted SP genes are listed in Table 3 by nucleotide position and locus tag ID based on the published annotation. The gene sizes of the Brucella suis surface proteins range from 93 to 3,000 base pairs.

Preparation of B. suis DNA: One milliliter of B. suis strain 1330 (ATCC 23444) bacteria was collected in a 50 ml tube and treated in an equal amount of phenol. After vortexing, the bacterial cells were spun down at 1,560 g, for 10 minutes at 4° C. Nine hundred μL of the supernatant was transferred into a 1.5 mL tube and 630 μL isopropanol was added. The sample was centrifuged at 16,000 g, for 10 minutes at 4° C. The pellet was washed with 70% alcohol and resuspended in 100 μL of sterile water.

Layout of the SP banks and PCR synthesis of B. suis SP genes: Two hundred and five genes that encoded surface protein of B. suis were selected with bioinformatic screen procedure for the gene synthesis. Primer sequences used for the surface antigens were designed and prepared in three 96 well plates as described in Example 1. The entire process for construction of the SP set from predicted gene sequence was implemented in a 96-well plate format to facilitate use of high-throughput methods wherever possible. The predicted SP genes, with their genome position, locus tag, predicted gene size, and their locations in three 96-well plates are shown in Table 3. The forward primers were designed with a CACC sequence at the 5′ end followed by the first 25 bases of the SP gene beginning with the start codon. The reverse primer was comprised of the last 25 bases of the SP gene prior to the stop codon. The PCR reaction was conducted in a final volume of 50 μL that contained 5 μL of 10× ThermalAce reaction buffer, 1 μL of 50 mM dNTP, 14 of 1:10 diluted B. suis template, 0.5 μL of each 50 μM primer, 41 μL of PCR water and 1 unit/μL of ThermalAce. Reactions of B. suis template without primers were used as a negative control. The lacZ gene with primers provided from Invitrogen was used as a positive control. The PCR running protocol was according to our previous study [13] designed as 1 cycle of 95° C. for 2 minutes, 30 cycles of 95° C. for 30 seconds, 55° C. for 30 seconds, 72° C. for 2.5 minutes, 1 cycle of 72° C. for 10 minutes and then refrigeration at 4° C. The presence and size of PCR products in each well were detected by 1% agarose gel electrophoresis with a 1 Kb DNA ladder (Invitrogen Inc) standard.

Cloning of surface genes into an expression vector: The PCR products were directly cloned into vector pET101/D-TOPO (Invitrogen, Inc). This vector includes a directional cloning site, a T7 lac promoter for high-level IPTG-inducible protein expression, coding sequences for ampicillin resistance, and coding sequences for C-terminal V5 epitope and hexahistidine (6His) expression. Reactions were performed according to the manufacturer's instructions. One ul of PCR product plus 1 ul of water was mixed with 0.5 ul of each salt solution and vector. Ligation reactions were conducted in 96 well plates at room temperature for 5 minutes according to the manufacturer's instructions. One μL of PCR product plus 1 μL of water were mixed with 0.5 μL of each salt solution and vector. The negative and positive transformation controls were pET101/D-TOPO vector alone and pET101/D-TOPO-LacZ, both from Invitrogen. Plasmid transformation was then conducted in 96 well plates. Two μL of the ligation reaction mixture was added to 40 μL of TOP10 competent cells per well and incubated at 4° C. for 30 minutes. After heating at 42° C. for 30 seconds, 250 μL of culture medium was added and the plates were shaken at 37° C. for 1 hour. After this incubation, 20 μL of the contents of each well was streaked on individual wells of 18 pre-warmed 12-well culture plates containing trypticase soy agar with 100 μg/mL ampicillin for clone selection. After plates were incubated at 37° C. overnight, a single colony from each well of the 12-well plates was picked and cultured in 1.2 mL LB broth medium with 100 μg/mL ampicillin in a 96-well deep-well plate for propagation of the recombinant plasmids at 37° C. overnight. On the next day, the plasmids were purified using a 96-well turbo Miniprep Spin column (Qiagen, Inc) and detected by electrophoresis on 1% agarose gel. The presence of inserted genes in the plasmids was detected by PCR using primers designed based on the sequence of the T7 promoter priming site (T7 forward: TAATA CGACT CACTA TAGGG) and the T7 reverse priming site (T7 Reverse: TAGTT ATTGC TCAGC GGTGG).

Expression of recombinant SPs: Competent BL21Star (DE3) E. coli cells were prepared with calcium for expression of B. suis SPs. Four μL of each purified plasmid preparation was mixed with 50 μL of expression host cells in a 96-well deep-well plate and incubated at 4° C. for 30 minutes. After heating at 42° C. for 45 seconds, 250 μL of culture medium was added into each well and the culture shaken at 37° C. for 1 hour. Then, 1 mL of LB solution containing 100 μg/mL of ampicillin was added and the culture was shaken at 37° C. overnight. On the next day, 250 μL of each culture was transferred into 5 mL of LB medium containing 1% glucose and 100 μg/mL ampicillin in 12 well plates and shaken at 37° C. for another 2 hours. Preliminary experiments indicated that this procedure would result in OD600 readings of the cultures between 0.4-0.8 units. IPTG (Invitrogen) was then added to a final 1 mM concentration and the plates were shaken for 5 hours at 37° C. After protein induction, cells were centrifuged at 1,560 g at 4° C. for 10 minutes. The cell pellets were dissolved in 500 μL of cell lysis buffer B-per solution (PIERCE Inc) for detection of protein. Protein expression was examined by both ELISA and Western blot methods.

Plate purification and detection of recombinant SPs by ELISA: A HisGrab plate (Pierce) was used to purify the recombinant surface proteins. Ten μL of the cell lysate and 90 μL of blotting buffer (10 mg casein in 900 mL of PBS, pH 7.4) were placed in wells of triplicate 96-well HisTag antibody plates (EMD Bioscience) and incubated at 4° C. overnight. After each well washed with 250 μL of PBS, 100 μL of a 1:2000 dilution of anti-V5 monoclonal antibody conjugated with alkaline phosphatase (Invitrogen Inc) was added to each well and incubated at 4° C. for 16 hours to detect the recombinant proteins. After the incubation, the plates were washed three times with 1×PBS before 100 μL of developing buffer containing 1 mg/mL Naphthol AS-MX phosphate disodium salt (Sigma) was added to each well. The plates were incubated at room temperature for 1 hour. Light transmission was measured at 410 nm single wavelength and the mean OD of corresponding wells in triplicate plates was taken as a semi-quantitative measure of protein concentration. The positive control was recombinant LacZ tagged with V5/6His and the negative control was medium alone. The OD of wells containing lysates from cells transfected with empty vector was less than 0.05. An OD reading of greater than 0.1 was considered to indicate the presence of recombinant protein.

Protein synthesis with TNT coupled reticulocyte lysate method: Brucella SPs that were not expressed by IPTG induction were synthesized by the TNT coupled reticulocyte lysate method using a reaction kit (Promega). One μg of pET101-SP plasmid was used as the DNA template. Twenty five μL TNT coupled reticulocyte lysate, 1 μL TNT RNA polymerase, 1 μL amino acid mixture (1 mM) and 1 μL RNasin (40 U/μL) were mixed in a 1.5 mL microcentrifuge tube and incubated at 30° C. for 90 minutes. The protein products were detected with WESTERN BLOT.

Column purification of recombinant SPs: Recombinant SPs were affinity purified from cell lysates via their 6His tag under native conditions using the Ni-NTA Superflow 96 BioRobot kit (Qiagen) according to the manufacturer's instructions. The cell pellets were resuspended in 100 μL of B-per solution (Pierce) and kept at room temperature for 30 minutes, then frozen and thawed 3 times. Soluble proteins went through a filter and were added to the Ni-NTA column. Insoluble proteins that were not solubilized in the B-per buffer were treated with inclusion body solubilization buffer (Pierce) and then added to the Ni-NTA column. After the columns were washed twice with NPI-20 buffer, recombinant proteins were eluted with 1 mL NPI-250 buffer into new 96-well deep well plates and stored at 4° C. until further analysis by Western blot.

Preparation of Rabbit Anti-Brucella IgG: Rabbit Anti-Brucella IgG was Used for screening of the recombinant Brucella surface proteins. Rabbits were immunized intramuscularly with dialyzed cell lysate of B. melitensis rough mutant WRR51 [14]. Research was conducted in compliance with the Animal Welfare Act and other federal statutes and regulations relating to animals and experiments involving animals and adhered to principles stated in the Guide for the Care and Use of Laboratory Animals, NRC Publication, 1996 edition. Rabbits were immunized intramuscularly with two doses of vaccine (25 μg bacterial lysate per dose) given 4 weeks apart. Control rabbits were injected intramuscularly with the same volume of normal saline. Blood was collected from the rabbits 2 weeks after the second dose of vaccine. IgG was prepared from the serum by affinity chromatography on protein G-Sepharose (Pharmacia-LKB Biotech), adjusted to 1.8 mg/mL, and used to screen SP for antigenicity by Western blot. (Antibodies elicited by immunization with B. melitensis also recognize B. suis proteins.)

Detection of recombinant SP by Western blot analysis Seven μL of each column-purified recombinant SP was mixed with an equal amount of 2×SDS loading buffer. The samples were boiled for 10 minutes and purified on 13% acrylamide/bis gel, then transferred onto nitrocellulose membrane and hybridized overnight with 1:2000 AP-conjugated anti-6His. The membranes were developed with 1 mg/mL of Naphthol AS-MX phosphate (Sigma) and 2 mg/mL Fast Red TR salt (Sigma) in 50 mM Tris buffer.

Screening of SP by Western blot with anti-Brucella antibody. Western blot assay was conducted as described above, except that rabbit anti-Brucella IgG was used.

### Results

Synthesis of B. suis SP genes by PCR Probable B. suis SP genes were predicted bioinformatically as described in the Materials and Methods, and a set of 205 predicted SP genes were selected for amplification, cloning and expression. The complete set of these SP is described in supporting information, Table 3.

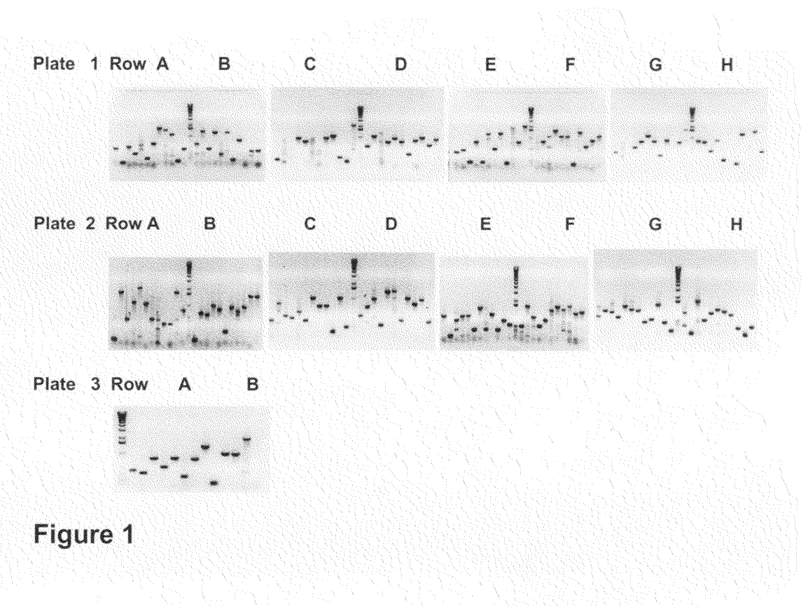

Molecular sizes of the predicted SP genes ranged from 93 by to 3000 bp. Purified genomic B. suis DNA prepared via phenol/chloroform isolation and detected on 1% agarose gel was used as the template for PCR synthesis of the SP ORFs. In preliminary experiments, we compared four DNA polymerases, pfx, pfu, Taq and ThermalAce (Invitrogen). All the polymerases produce blunt-end PCR products and perform 3′ end proofreading. ThermalAce provided the highest number of positive PCR products and was used to produce the data depicted in FIG. 1. We prospectively established an iterative strategy using three successive rounds of synthesis to maximize the proportion of products synthesized with high fidelity, but to limit the number of rounds of synthesis and to avoid adjustment of PCR conditions for each individual gene. The first round of synthesis, conducted using the conditions of PCR synthesis described in the methods, led to PCR products of the predicted size for 182 of 205 genes (89%) (FIG. 1). In the remaining 23 genes, 10 PCR products were represented as a single band of incorrect size. Thirteen genes (6%) were not amplified in the first round of the PCR assay. Genes that did not show products of the correct predicted size after the first round were subjected to one or two further rounds of synthesis in which the annealing temperature was reduced to 53° C. and 52° C., respectively. Of the 23 genes that underwent a second round of synthesis, 15 (65%) showed a product. The third round resulted in products for 1 out of 8 genes (13%). Overall, as assessed by presence of a band on agarose gel, 198 out of 205 genes (97%) of the predicted size were detected after three rounds of PCR.

Construction of an expression library of B. suis SP genes: The contents of all 205 wells of PCR-synthesized Brucella SP genes, whether product was demonstrated by agarose gel electrophoresis or not, were directly cloned into an expression vector, pET101/D-TOPO (Invitrogen, Inc), and propagated in TOP10 cells according to the manufacturer's instructions as described in the methods. Recombinant cells were selected on 18 12-well culture plates of LB agar containing 100 μg/mL ampicillin. One well was used for one SP. In the first round of cloning, 201/205 surface antigens (98%) resulted in isolated colonies on the selection plates. Another two rounds of cloning were conducted for the remaining four genes, but not even a single colony was obtained from these clonings. All 198 genes with PCR product detected by agarose gel electrophoresis were present in the set of 201 cloned genes.

FIG. 1 shows agarose gel confirmation of B. suis SP genes synthesized by PCR Synthesis of 205 B. suis SP genes was attempted using three separate rounds of PCR. PCR products were electrophoresed on 1% agarose gel to examine their molecular sizes. A 1 Kb ladder (Invitrogen) was used as the standard. Product was detected in 198/205 lanes after three rounds of PCR.

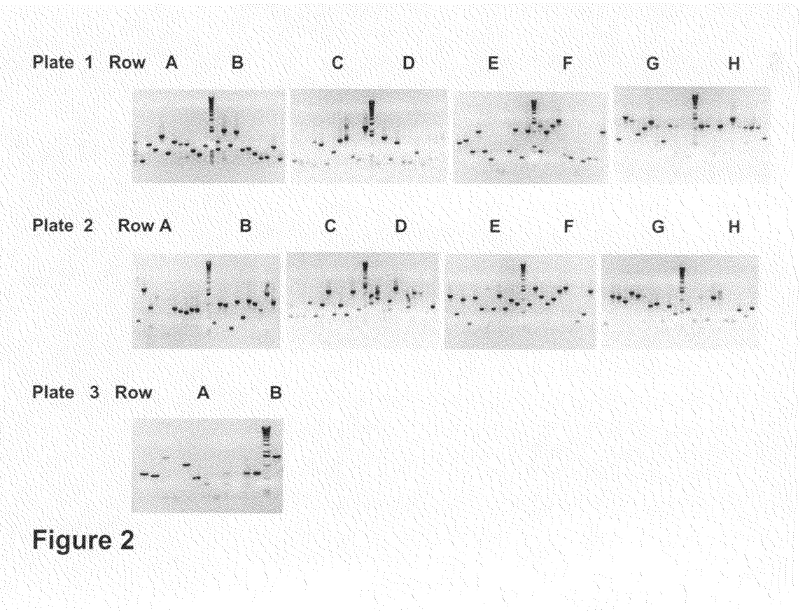

A single colony from each well was picked and cultured for plasmid isolation in LB broth. Plasmids from the cultures were purified with a Turbo miniprep method and examined by PCR analysis using forward and reverse primers based on T7. The PCR products were examined by electrophoresis on a 1% agarose gel and 185 plasmids out of 205 SP genes (90%) were found to have inserts with the correct molecular size (FIG. 2). The remaining 16 plasmids either had no detectable insert or an insert with an incorrect size. The full length sequences of the gene inserts of the plasmids were further confirmed by sequencing using ABI 3730xl capillary sequencing machines at The Institute of Genomic Research (TIGR). All of the recombinant plasmids tested contained a single insert that exactly matched its predicted sequence. Recombinant TOP10 cells with confirmed inserts of the correct sequence were prepared in 20% glycerol stock in 96 deep well plates and referred to as the B. suis SP Gene Bank. The positive plasmids were also stored for future studies.

FIG. 2. shows PCR identification of plasmids using primers located at T7 priming sites. Recombinant plasmids were identified by 1% agarose gel electrophoresis of 5 μL of PCR products synthesized using primers based on the T7 priming site sequence located on the pET101/D-TOPO vector. A 1 Kb ladder was used as standard (middle lane for Plates 2 and 3, second lane from the right for Plate 3).

Protein expression and purification: After IPTG-induced recombinant protein expression, the presence of recombinant proteins was assessed in crude cell lysates by both ELISA for detection of the V5 tag and Western blot analysis for detection of the 6-His tag (Table 3). Recombinant LacZ protein fused with 6His N5 tags was used as a positive control for both assay methods. An OD value of 0.10 in the ELISA was used as the minimum value that indicated the presence of recombinant protein, indicated by a P in Table 3. Proteins that showed a visually detectable band on Western blot are marked with a P in the respective column and those that showed no band are marked N; a question mark indicates that 10 proteins showed bands of a size different than that predicted from the ORF analysis. The number of expressed recombinant SP detected by these methods is summarized in Table 1.

TABLE 1
Detection of Recombinant SPs by ELISA and Western Blot

| | | Number (%) of Positive Wells by Detection Method | | | | | |
|---|---|---|---|---|---|---|---|
| | Wells | | Western | | | | |
| Plate | Tested | ELISA | Blot | Both | Either | Neither | IB^{a} |
| Plate 1 | 96 | 77(80%) | 83(87%) | 75(78%) | 84(88%) | 12(13%) | 12(13%) |
| Plate 2 | 96 | 81(84%) | 85(89%) | 79(82%) | 88(92%) | 8(8%) | 18(19%) |
| Plate 3 | 13 | 13(100%) | 13(100%) | 13(100%) | 13(100%) | 0(0%) | 1(8%) |
| Total | 205 | 171(83%) | 181(88%) | 167(81%) | 185(90%) | 20(10%) | 31(15%) |

^{a}SP present in inclusion bodies

Proteins were detected by ELISA in 171/205 wells (83%) and by Western blot in 181/205 wells (88%). Proteins were detected in 167/205 wells (81%) by both methods and in 185/205 wells (90%) by either method. Fifteen SPs were positive only by Western blot and 3 were positive only by ELISA. A total of 20 out of 205 wells (10%) were negative by both ELISA and Western blot. The percentage of positive wells detected by one, both or either method was generally similar across all three plates. Of the 181 recombinant proteins detected by Western blot, 31 represented proteins present in inclusion bodies.

The 20 plasmids that did not express SPs were used as templates to conduct TNT protein synthesis. Three expressed genes (marked C in the TNT column of Table 3) were used as positive controls to monitor the TNT protein synthesis; all 3 produced recombinant proteins. Of the 20 non-expressed genes, 7 (marked with a P in the TNT column) produced proteins by the TNT method. After the presence of protein was confirmed, cultures from positive wells were expanded and stored in 20% glycerol in 96 well deep well plates and referred to as the B. suis SP protein bank.

Screening recombinant proteins for immune reactivity against anti-Brucella rabbit IgG: We investigated the Brucella-specific seroreactivity of the 167 recombinant Brucella proteins detected by both anti-6His Western Blot and anti-V5 ELISA, and two additional proteins selected. The 169 selected proteins were column purified and concentrated after expression from the B. suis SP protein bank as described in the methods. Five of the expressed proteins (Table 2) were detected by Western blot analysis using rabbit anti-Brucella IgG. SodC is a periplasmic protein in B. abortus [15]. None of the other proteins have been demonstrated to be cell surface or secreted proteins.

TABLE 2
Brucella-Reactive Recombinant SPs

| | Locus | |
|---|---|---|
| Cell | Tag | Protein Features |
| 01B3 | BRA0464 | Peptide ABC transporter, ATP-binding protein. Protein |
| | | expressed is 3′ 474 of 906 nucleotides |
| 01C5 | BR0501 | Hypothetical protein with features of polysaccharide |
| | | deacetylase, lipoprotein and serine recombinase; |
| | | calculated MW = 29025 |
| 01E7 | BRA0703 | Cu—Zn superoxide dismutase(SodC); |
| | | calculated MW = 18000; periplasmic protein |
| | | in B. abortus |
| 01G2 | BR0611 | Serine protease, calculated MW = 53352 |
| 03A1 | BR1074 | NifU-related protein; calculated MW = 15958 |

It is obviously difficult, even impossible, by using traditional cloning technologies to construct the enormous number of genes from the simplest organism and evaluate their multiple functional products within a short time. The High Throughput (HT) concept has provided useful technologies that will be necessary to harness the informatic power of genome sequencing. We have previously described the development of conditions for the HECES HT method [13]. In this report, we have modified and extended the HECES to express and screen the Brucella SPs as a model approach to whole-genome functional screening. We expressed a high proportion of the selected genes using pre-defined conditions that were applied across the entire gene set. The HECES includes three iterations of PCR cloning at pre-set conditions, with descending stringency based on annealing temperature. This approach led to full-length cloning of 198/205 genes (96.6%) with no requirement to adjust individual conditions for each gene.

As expected, HT protein expression was more difficult, given the variety of challenges encountered in translation, solubilization, secretion and potential protein toxicity for the expression host. In addition to these issues, which may affect the production of proteins of correct sequence, proper protein folding is required for a number of functional tests. By their nature, HT approaches do not permit much flexibility in tailoring reaction conditions to optimize expression of each protein in a conformationally correct form. We focused on protein solubility as an important determinant for extraction from the host cells and separated proteins first with non-denaturing conditions and then used denaturing conditions for insoluble proteins. Extraction using denaturing conditions resulted in purification of 31 of the 181 proteins that were positive by Western blot and increased the overall protein yield by 15% (31/205 genes). The modified HECES we used in this study was highly successful for construction of the SP205 protein library. Overall, protein was expressed from 81% to 90% of in silico-predicted SP genes (depending on the criteria used for positivity) using only two variations in expression and extraction conditions. The higher number may be more accurate, since the quantity of protein in some of the ELISA-positive wells may have been too little to detect by WESTERN BLOT. The failure of protein expression did not appear to be related to gene size (data not shown).

HT expression of protein from a higher percentage of genes should be possible, but cost may increase considerably. Interestingly, when we attempted in vitro TNT translation from the genes that did not express proteins in BL21 Star cells, only 7 (35%) yielded products, while all three of the control genes were expressed. Although this study was not primarily designed to assess the relative merits of in vivo and in vitro translation, this observation suggests that use of in vivo translation at the single set of conditions we chose would not overcome all the limitations of our in vivo approach. Although the addition of in vitro translation as a conditionally programmed HT step (instead of ad hoc as we did) may be useful to increase the number of expressed proteins, the added benefit in our study would have been only 3.4% (7/205 genes). The value of additional iterations of in vitro translation with variations in experimental conditions or further optimization of the conditions could be evaluated in later studies. The HECES would provide an excellent benchmark for assessing the relative efficiencies of in vivo and in vitro translation systems under a variety of experimental conditions for a large protein set.

This project was originally designed to produce proteins that were arrayed in a format suitable for discovery of vaccine or diagnostic antigens suitable for further development to combat brucellosis. For analysis of Brucella seroreactivity, we initially planned to capture proteins from the crude lysate directly on an ELISA plate by use of either chelation of 6His to nickel or by antibody-mediated binding of the V5 tag. In preliminary studies (data not shown), we found that there was variability in binding using the two different methods. After applying the same amount of purified recombinant proteins on the two kinds of plates, OD readings over baseline on the anti-6His antibody plate were at least 2 fold higher than those observed with the Nickel-chelating plate. Due to this variability, we included a Ni-NTA column chromotography step to purify and concentrate proteins prior to probing with anti-Brucella IgG. We used native conditions for the purification, since our primary intent was to screen the library for immunogenicity with antibodies, which optimally requires maintenance of the native protein conformation. Using this column, we obtained approximately 20 to 300 μg per 5 mL bacterial culture. This large range of protein yield may reflect the wide range of protein solubility and binding properties as well as variability in protein expression under relatively uniform conditions. For production of libraries for purposes that do not require retention of native protein conformation, denaturation and purification via the 6His tag should be considered as it may yield a higher success rate. For our purpose, the absolute quantity of protein was not crucial, since only small amounts were required for studies of antigenicity.

In preliminary studies (data not shown), we arrayed the proteins on two microtiter plates after adjustment for protein concentration. Plates were screened by ELISA using the rabbit antibody employed for the anti-Brucella Western blot and compared with IgG prepared from non-immune rabbits. Although a number of additional proteins were reactive in this assay, there was significant variability among repeat experiments. Additional experiments are in progress to refine this assay for plate-based HT screening. Screening of all 167 SP by Western blot resulted in detection of five proteins, one of which (SodC) has been localized in the periplasmic space of B. abortus [15]. These Western blot results confirmed the utility of the HECES for HT cloning and expression of immunoreactive Brucella proteins. The five proteins we identified, as well as the likely additional immunoreactive proteins, may have utility as diagnostic or vaccine antigens for brucellosis.

The technology platform used to produce the large number of Brucella proteins in this study is a modification of the HECES we previously reported [13]. The HECES was designed to be compatible with either automated systems or benchtop methods using a 96-well plate format. In our previous report, we used Invitrogen's TOPO DNA topoisomerase I system for cloning PCR products into a Gateway entry vector, followed by cloning into a Gateway destination vector. For preparation of the SP205 protein library, we improved the HECES by cloning PCR products directly into an expression vector using the TOPO directional cloning system. This approach reduced the number of steps in the overall process, with associated reduction in time and cost. This single-step strategy not only saves working time but also avoids many potential problems that may impact the cloning success rate. The Gateway system is highly flexible for later application of the gene library, since it allows use of different destination vectors and promoters that can be applied to different hosts. This limitation is counterbalanced for our requirements by the improved efficiency in reaching the main goal of developing the protein library.

Our success rate in expression of the SP205 protein library is consistent with our previously reported success rate of 90% (18/20 proteins) using the Gateway-based HECES [13] for preparation of proteins of varied size and functions. The tradeoffs between high throughput and the need to individualize reaction conditions are most evident at the protein expression and purification steps. The SP205 protein library represents a reasonable compromise among cost, time and utility. Although it is likely that additional seroreactive proteins may be present in the remainder of the complete B. suis ORFeome), the positive results we obtained are sufficient to enable a number of additional studies directed toward development of new diagnostic tests and vaccines using the putative surface antigens identified and enumerated herein.

### Potential Commercial Applications of the Invention:

The recombinant constructs can be used to express a complete set of putative Brucella surface antigens. The recombinant constructs are amplified and purified from their recombinant cells. Both recombinant constructs and cells are prepared in a 96 well plate format by direct transferring between multi-well plates. These plates can be used as final products directly to provide to scientists of universities, institutes, and industrial companies for their specific screening and array studies. Once any individual, specific antigen is selected during the studies, these antigens can also be prepared in a large scale from the recombinant library and purified using convenient 6-His and V5 purification handles located on C terminal of each recombinant constructs. The plates that contain either recombinant constructs or cells can be fast prepared with low coast and be easily storage.

Table 3 shows detection and anti-Brucella seroreactivity of recombinant B. suis SPs.

TABLE 3
Detection and anti-Brucella Seroreactivity of Recombinant B. suis SPs

| Sequence | | | | Locus | anti-V5 | Anti-6His | | | Anti-Bru |
|---|---|---|---|---|---|---|---|---|---|
| ID number | Well | Chromosome | Amplicon position | Tag^{a} | ELISA OD | WB^{b} | IB^{c} | TNT^{d} | WB positive^{e} |
| 1 | 1A1 | NC_004310 | 493606 . . . 494130 | BR0494 | 0.1 | P | | | |
| 2 | 1A2 | NC_004310 | 1464671 . . . 1464778 | — | 0.05 | N | | | |
| 3 | 1A3 | NC_004310 | 1464025 . . . 1464603 | BR1511 | 0.21 | P | | | |
| 4 | 1A4 | NC_004310 | 1463752 . . . 1463417 | BR1510 | 0.22 | P | | | |
| 5 | 1A5 | NC_004310 | 1459484 . . . 1458876 | BR1504 | 0.1 | P | Y | | |
| 6 | 1A6 | NC_004310 | 1450538 . . . 1450729 | BR1492 | 0.11 | P | | | |
| 7 | 1A7 | NC_004310 | 202754 . . . 202083 | — | 0.05 | P | | | |
| 8 | 1A8 | NC_004310 | 191982 . . . 193649 | BR0181 | 0.13 | P | | | |
| 9 | 1A9 | NC_004310 | 190154 . . . 191638 | BR0179 | 0.16 | P | Y | | |
| 10 | 1A10 | NC_004311 | 451255 . . . 452391 | BRA0465 | 0.08 | N | | | |
| 11 | 1A11 | NC_004310 | 179170 . . . 179054 | — | 0.1 | N | | | |
| 12 | 1A12 | NC_004310 | 177676 . . . 178137 | BR0161 | 0.86 | P | | | |
| 13 | 1B1 | NC_004310 | 172931 . . . 173584 | BR0156 | 0.74 | P | | | |
| 14 | 1B2 | NC_004311 | 418657 . . . 420042 | BRA0432 | 0.31 | P | | | |
| 15 | 1B3 | NC_004311 | 450782 . . . 451255 | BRA0464 | 0.64 | P | | | Y |
| 16 | 1B4 | NC_004310 | 902486 . . . 903775 | BR0930 | 0.1 | P | | | |
| 17 | 1B5 | NC_004310 | 908982 . . . 906232 | — | 0.33 | P | | | |
| 18 | 1B6 | NC_004310 | 906319 . . . 907593 | BR0933 | 0.92 | P | | | |
| 19 | 1B7 | NC_004311 | 253475 . . . 253326 | BRA0272 | 0.2 | P | | | |
| 20 | 1B8 | NC_004310 | 307045 . . . 306206 | BR0295 | 0.1 | P | | | |
| 21 | 1B9 | NC_004310 | 382841 . . . 383596 | BR0371 | 0.05 | P | | | |
| 22 | 1B10 | NC_004310 | 357604 . . . 356180 | BR0347 | 0.1 | P | | | |
| 23 | 1B11 | NC_004311 | 217431 . . . 217877 | — | 0.06 | N | | P | |
| 24 | 1B12 | NC_004310 | 845101 . . . 845961 | BR0874 | 0.23 | P | Y | | |
| 25 | 1C1 | NC_004310 | 808237 . . . 808049 | — | 1.07 | N | | | |
| 26 | 1C2 | NC_004310 | 778276 . . . 778151 | BR0800 | 1.19 | P | | | |
| 27 | 1C3 | NC_004311 | 859344 . . . 859124 | — | 0.24 | P | | | |
| 28 | 1C4 | NC_004311 | 43164 . . . 42172 | BRA0046 | 0.1 | P | | | |
| 29 | 1C5 | NC_004310 | 503088 . . . 503909 | BR0501 | 0.1 | P | | | Y |
| 30 | 1C6 | NC_004311 | 276810 . . . 277811 | — | 0.03 | N | | P | |
| 31 | 1C7 | NC_004311 | 15372 . . . 16478 | BRA0017 | 0.1 | P | | | |
| 32 | 1C8 | NC_004311 | 263773 . . . 264777 | BRA0280 | 0.57 | P | | | |
| 33 | 1C9 | NC_004310 | 533776 . . . 534882 | BR0540 | 0.1 | P | | | |
| 34 | 1C10 | NC_004310 | 535578 . . . 535369 | BR0541 | 0.06 | P | Y | | |
| 35 | 1C11 | NC_004310 | 423995 . . . 423891 | BR0419 | 0.11 | P | Y | | |
| 36 | 1C12 | NC_004310 | 577664 . . . 575511 | BR0583 | 0.1 | P | | | |
| 37 | 1D1 | NC_004310 | 469881 . . . 470483 | BR0471 | 1.04 | P | | | |
| 38 | 1D2 | NC_004310 | 472847 . . . 473962 | BR0475 | 1.05 | P? | | | |
| 39 | 1D3 | NC_004310 | 602362 . . . 603762 | BR0613 | 0.43 | P | Y | | |
| 40 | 1D4 | NC_004310 | 476907 . . . 476110 | BR0479 | 0.86 | P | | | |
| 41 | 1D5 | NC_004310 | 614828 . . . 615841 | BR0618 | 0.1 | N | | | |
| 42 | 1D6 | NC_004310 | 701720 . . . 700902 | BR0716 | 0.1 | P | Y | | |
| 43 | 1D7 | NC_004311 | 39971 . . . 39612 | BRA0043 | 0.22 | P | | | |
| 44 | 1D8 | NC_004310 | 665595 . . . 666512 | BR0680 | 0.4 | P | Y | | |
| 45 | 1D9 | NC_004311 | 404880 . . . 403813 | BRA0419 | 0.21 | P? | | | |
| 46 | 1D10 | NC_004311 | 410713 . . . 410036 | — | 0.05 | P | | | |
| 47 | 1D11 | NC_004310 | 1916348 . . . 1917310 | BR1988 | 0.48 | P | | | |
| 48 | 1D12 | NC_004310 | 1918501 . . . 1919070 | BR1991 | 0.96 | P? | | | |
| 49 | 1E01 | NC_004310 | 777806 . . . 777408 | BR0799 | 1.4 | P | | | |
| 50 | 1E02 | NC_004311 | 932388 . . . 932861 | BRA0944 | 0.12 | P | Y | | |
| 51 | 1E03 | NC_004311 | 218638 . . . 218495 | — | 0.2 | P | | | |
| 52 | 1E04 | NC_004310 | 843444 . . . 844244 | BR0872 | 0.1 | P | | | |
| 53 | 1E05 | NC_004311 | 689483 . . . 689336 | — | 0.02 | N | | | |
| 54 | 1E06 | NC_004311 | 685682 . . . 686959 | BRA0706 | 0.1 | P | | | |
| 55 | 1E07 | NC_004311 | 683096 . . . 682575 | BRA0703 | 0.76 | P | | | Y |
| 56 | 1E08 | NC_004311 | 680986 . . . 682311 | BRA0702 | 0.46 | P | | | |
| 57 | 1E09 | NC_004311 | 680814 . . . 680680 | — | 0.04 | P | | | |
| 58 | 1E10 | NC_004311 | 694385 . . . 696271 | BRA0714 | 0.28 | P | | | |
| 59 | 1E11 | NC_004311 | 698826 . . . 698029 | BRA0717 | 0.11 | P | | | |
| 60 | 1E12 | NC_004310 | 1528243 . . . 1529076 | BRA0719 | 0.26 | P | | | |
| 61 | 1F1 | NC_004311 | 0718794 . . . 0719963 | BRA0734 | 0.1 | P | | | |
| 62 | 1F2 | NC_004310 | 1528243 . . . 1529076 | BR1580 | 0.02 | P | | | |
| 63 | 1F3 | NC_004310 | 923026 . . . 921905 | — | 0.81 | P? | | | |
| 64 | 1F4 | NC_004310 | 949390 . . . 950946 | BR0982 | 0.1 | P | | | |
| 65 | 1F5 | NC_004310 | 932042 . . . 930939 | BR0960 | 0.31 | P | | | |
| 66 | 1F6 | NC_004310 | 16123 . . . 17226 | — | 0.03 | P | Y | | |
| 67 | 1F7 | NC_004310 | 15886 . . . 15996 | — | 0.4 | P | Y | | |
| 68 | 1F8 | NC_004310 | 2104646 . . . 2103267 | BR2181 | 1.94 | P | | | |
| 69 | 1F9 | NC_004310 | 987835 . . . 988434 | BR1022 | 0.77 | P | | | |
| 70 | 1F10 | NC_004310 | 2102842 . . . 2101973 | BR2179 | 0.1 | P | | | |
| 71 | 1F11 | NC_004310 | 1031664 . . . 1030567 | BR1060 | 0.62 | P | | | |
| 72 | 1F12 | NC_004310 | 1620750 . . . 1620643 | BR1678 | 0.62 | P | | | |
| 73 | 1G1 | NC_004310 | 1602583 . . . 1602188 | BR1651 | 0.3 | P | | | |
| 74 | 1G2 | NC_004310 | 600020 . . . 601558 | BR0611 | 1.18 | P | | | Y |
| 75 | 1G3 | NC_004310 | 1094409 . . . 1092088 | BR1116 | 0.7 | P | | | |
| 76 | 1G4 | NC_004310 | 583447 . . . 584010 | BR0590 | 1.1 | P | | | |
| 77 | 1G5 | NC_004310 | 566507 . . . 567313 | BR0572 | 0.32 | P | | | |
| 78 | 1G6 | NC_004310 | 1137143 . . . 1136007 | BR1156 | 0.09 | N | | | |
| 79 | 1G7 | NC_004310 | 1138022 . . . 1137213 | BR1157 | 0.1 | P | | | |
| 80 | 1G8 | NC_004310 | 529028 . . . 528710 | — | 0.48 | P | | | |
| 81 | 1G9 | NC_004310 | 249420 . . . 250301 | BR0237 | 0.03 | P | | | |
| 82 | 1G10 | NC_004310 | 249132 . . . 247108 | — | 0.15 | P | | | |
| 83 | 1G11 | NC_004310 | 246965 . . . 246174 | BR0234 | 0.07 | N | | P | |
| 84 | 1G12 | NC_004310 | 241645 . . . 244644 | BR0231 | 0.1 | P | | | |
| 85 | 1H1 | NC_004310 | 1257866 . . . 1258747 | — | 0.37 | P | Y | | |
| 86 | 1H2 | NC_004310 | 1242836 . . . 1243660 | BR1282 | 0.07 | N | | | |
| 87 | 1H3 | NC_004310 | 1251755 . . . 1251291 | BR1290 | 0.02 | P | | | |
| 88 | 1H4 | NC_004310 | 994615 . . . 993719 | — | 1.14 | P | | | |
| 89 | 1H5 | NC_004310 | 969447 . . . 969232 | — | 0.1 | P? | | | |
| 90 | 1H6 | NC_004310 | 968529 . . . 967123 | BR1004 | 0.11 | P | | | |
| 91 | 1H7 | NC_004310 | 963424 . . . 963555 | BR1000 | 0.14 | P | | | |
| 92 | 1H8 | NC_004310 | 656492 . . . 657823 | BR0668 | 0.28 | P | | | |
| 93 | 1H9 | NC_004310 | 663564 . . . 665345 | BR0678 | 0.1 | P | | | |
| | | | | and | | | | | |
| | | | | BR0679 | | | | | |
| 94 | 1H10 | NC_004310 | 1349868 . . . 1348297 | BR1394 | 0.1 | P | | | |
| 95 | 1H11 | NC_004310 | 2043737 . . . 2043288 | BR2120 | 0.06 | N | | P | |
| 96 | 1H12 | NC_004310 | 1383558 . . . 1382239 | BR1426 | 1.26 | P | | | |
| 97 | 2A1 | NC_004310 | 2030862 . . . 2030992 | BR2104 | 0.31 | P? | | | |
| 98 | 2A2 | NC_004310 | 1397867 . . . 1396047 | BR1437 | 0.42 | P | | | |
| 99 | 2A3 | NC_004310 | 2013200 . . . 2013775 | BR2090 | 0.11 | P | | | |
| 100 | 2A4 | NC_004310 | 676289 . . . 675126 | BR0687 | 0.05 | N | | | |
| 101 | 2A5 | NC_004310 | 687198 . . . 685093 | BR0699 | 0.17 | P | | | |
| 102 | 2A6 | NC_004310 | 1458866 . . . 1457805 | BR1503 | 0.1 | P | | | |
| 103 | 2A7 | NC_004310 | 689605 . . . 689060 | BR0702 | 0.19 | P | | | |
| 104 | 2A8 | NC_004310 | 1473392 . . . 1474687 | BR1522 | 0.1 | P? | Y | | |
| 105 | 2A9 | NC_004311 | 954323 . . . 954757 | — | 0.1 | P | | | |
| 106 | 2A10 | NC_004311 | 953747 . . . 954196 | — | 0.04 | P | Y | | |
| 107 | 2A11 | NC_004310 | 1566008 . . . 1567762 | BR1620 | 0.05 | N | | | |
| 108 | 2A12 | NC_004311 | 497429 . . . 498265 | BRA0519 | 0.1 | P | Y | | |
| 109 | 2B1 | NC_004310 | 1582367 . . . 1582242 | BR1635 | 0.25 | P | | | |
| 110 | 2B2 | NC_004311 | 1199692 . . . 1200417 | BRA1197 | 0.2 | P | | | |
| 111 | 2B3 | NC_004311 | 1160261 . . . 1159590 | BRA1160 | 0.27 | P | | | |
| 112 | 2B4 | NC_004310 | 1622599 . . . 1621619 | BR1679 | 0.1 | P | | | |
| 113 | 2B5 | NC_004310 | 96925 . . . 96092 | BR0091 | 0.53 | P | | | |
| 114 | 2B6 | NC_004311 | 1122685 . . . 1122422 | BRA1133 | 0.01 | N | | P | |
| 115 | 2B7 | NC_004310 | 1119290 . . . 1120198 | BR1141 | 0.36 | P | | | |
| 116 | 2B8 | NC_004311 | 721117 . . . 721893 | BRA0737 | 0.1 | P | | | |
| 117 | 2B9 | NC_004311 | 1001108 . . . 1000158 | BRA1011 | 0.48 | P | Y | | |
| 118 | 2B10 | NC_004311 | 1150779 . . . 1152314 | BRA1151 | 0.84 | P | | | |
| 119 | 2B11 | NC_004311 | 1188366 . . . 1186843 | BRA1865 | 0.45 | P | | | |
| 120 | 2B12 | NC_004311 | 1190242 . . . 1189448 | BRA1187 | 0.17 | P | | | |
| 121 | 2C1 | NC_004311 | 932669 . . . 933049 | BRA0944 | 0.22 | P | | | |
| 122 | 2C2 | NC_004310 | 1811930 . . . 1811052 | BR1873 | 0.21 | P | | | |
| 123 | 2C3 | NC_004311 | 144434 . . . 144904 | BRA0155 | 0.26 | P | Y | | |
| 124 | 2C4 | NC_004311 | 868452 . . . 868033 | BRA0885 | 0.47 | P | | | |
| 125 | 2C5 | NC_004311 | 866546 . . . 865974 | BRA0881 | 0.11 | N | | | |
| 126 | 2C6 | NC_004311 | 865343 . . . 864993 | BRA0879 | 0.16 | P | | | |
| 127 | 2C7 | NC_004311 | 108011 . . . 106965 | BRA0110 | 0.24 | P | | | |
| 128 | 2C8 | NC_004311 | 103775 . . . 104518 | BRA0107 | 0.1 | P | | | |
| 129 | 2C9 | NC_004311 | 810953 . . . 811692 | — | 0.03 | N | | | |
| 130 | 2C10 | NC_004311 | 1041455 . . . 1041559 | — | 0.02 | N | | P | |
| 131 | 2C11 | NC_004311 | 1032857 . . . 1033900 | BRA1049 | 0.1 | P | | | |
| 132 | 2C12 | NC_004310 | 85987 . . . 86178 | BR0079 | 0.1 | P | Y | | |
| 133 | 2D1 | NC_004311 | 725632 . . . 726084 | — | 0.18 | P | Y | | |
| 134 | 2D2 | NC_004311 | 627456 . . . 626746 | — | 0.31 | P | | | |
| 135 | 2D3 | NC_004310 | 1067800 . . . 1068804 | — | 0.78 | P | Y | | |
| 136 | 2D4 | NC_004310 | 1070094 . . . 1069858 | BR1096 | 0.37 | P | | | |
| 137 | 2D5 | NC_004310 | 2017548 . . . 2018945 | BR2097 | 0.1 | P | Y | | |
| 138 | 2D6 | NC_004310 | 1831258 . . . 1828637 | BR1895 | 0.06 | N | | | |
| 139 | 2D7 | NC_004310 | 2060053 . . . 2059700 | BR2135 | 0.35 | P | | | |
| 140 | 2D8 | NC_004310 | 1822616 . . . 1823692 | BR1888 | 0.21 | N | | | |
| 141 | 2D9 | NC_004311 | 629978 . . . 630844 | BRA0649 | 0.07 | P | Y | | |
| 142 | 2D10 | NC_004310 | 1821550 . . . 1822590 | BR1887 | 0.15 | P | | | |
| 143 | 2D11 | NC_004311 | 599116 . . . 599478 | BRA0613 | 0.41 | P | | | |
| 144 | 2D12 | NC_004310 | 1984295 . . . 1983501 | BR2059 | 0.98 | P | | | |
| 145 | 2E01 | NC_004310 | 1961782 . . . 1960766 | BR2036 | 0.17 | P | | | |
| 146 | 2E02 | NC_004311 | 18423 . . . 18187 | BRA0020 | 0.53 | P | | | |
| 147 | 2E03 | NC_004310 | 1941591 . . . 1942403 | BR2016 | 0.36 | P | | | |
| 148 | 2E04 | NC_004310 | 761432 . . . 762400 | BR0777 | 0.51 | P | | | |
| 149 | 2E05 | NC_004310 | 1922413 . . . 1921514 | BR1995 | 0.12 | P | | | |
| 150 | 2E06 | NC_004310 | 762459 . . . 762842 | BR0778 | 0.89 | P | | | |
| 151 | 2E07 | NC_004310 | 1909342 . . . 1907945 | — | 0.05 | N | | P | |
| 152 | 2E08 | NC_004311 | 76331 . . . 75954 | BRA0077 | 0.18 | P | | | |
| 153 | 2E09 | NC_004311 | 103529 . . . 104518 | BRA0107 | 0.16 | P | | | |
| 154 | 2E10 | NC_004310 | 1845490 . . . 1845131 | BR1911 | 0.14 | N | | | |
| 155 | 2E11 | NC_004311 | 146843 . . . 147556 | BRA0158 | 0.45 | P | | | |
| 156 | 2E12 | NC_004310 | 1768232 . . . 1767684 | BR1836 | 0.21 | P | Y | | |
| 157 | 2F1 | NC_004310 | 1739527 . . . 1739000 | BR1809 | 0.2 | P | Y | | |
| 158 | 2F2 | NC_004310 | 1727960 . . . 1726398 | BR1799 | 0.7 | P | | | |
| 159 | 2F3 | NC_004310 | 1721473 . . . 1720568 | BR1791 | 1.3 | P | | | |
| 160 | 2F4 | NC_004311 | 263237 . . . 263773 | BRA0279 | 1.01 | P | | | |
| 161 | 2F5 | NC_004310 | 1653151 . . . 1653939 | BR1711 | 0.15 | P | | | |
| 162 | 2F6 | NC_004310 | 1642494 . . . 1641166 | BR1697 | 0.14 | P? | | | |
| 163 | 2F7 | NC_004310 | 1629788 . . . 1628190 | BR1685 | 0.36 | P | | | |
| 164 | 2F8 | NC_004311 | 363804 . . . 365234 | BRA0384 | 0.1 | P | | | |
| 165 | 2F9 | NC_004310 | 1566005 . . . 1567762 | BR1620 | 0.01 | P | | | |
| 166 | 2F10 | NC_004311 | 406415 . . . 407647 | BRA0421 | 0.04 | P | Y | | |
| 167 | 2F11 | NC_004310 | 1561849 . . . 1563135 | BR1614 | 0.23 | P | | | |
| 168 | 2F12 | NC_004310 | 1559879 . . . 1558866 | BR1612 | 0.76 | P | | | |
| 169 | 2G1 | NC_004310 | 1556547 . . . 1555729 | BR1609 | 0.13 | P | | | |
| 170 | 2G2 | NC_004311 | 414537 . . . 413413 | BRA0428 | 0.16 | N | | | |
| 171 | 2G3 | NC_004311 | 414830 . . . 415875 | BRA0429 | 0.13 | P | | | |
| 172 | 2G4 | NC_004310 | 1549188 . . . 1548409 | BR1605 | 0.2 | P | | | |
| 173 | 2G5 | NC_004311 | 418666 . . . 420042 | BRA0432 | 0.21 | P | Y | | |
| 174 | 2G6 | NC_004310 | 1517997 . . . 1519133 | — | 0.13 | P? | Y | | |
| 175 | 2G7 | NC_004311 | 457997 . . . 459043 | BRA0472 | 0.07 | P | Y | | |
| 176 | 2G8 | NC_004310 | 1503689 . . . 1503186 | BR1554 | 0.17 | P | | | |
| 177 | 2G9 | NC_004311 | 477228 . . . 476632 | BRA0497 | 0.35 | P | Y | | |
| 178 | 2G10 | NC_004310 | 1455667 . . . 1457064 | BR1500 | 0.08 | N | | | |
| 179 | 2G11 | NC_004310 | 1439152 . . . 1438703 | BR1482 | 0.59 | P | Y | | |
| 180 | 2G12 | NC_004310 | 1415777 . . . 1415601 | BR1457 | 0.29 | P? | | | |
| 181 | 2H1 | NC_004311 | 576909 . . . 575983 | BRA0590 | 0.13 | P | | | |
| 182 | 2H2 | NC_004310 | 760169 . . . 760077 | BR0775 | 0.8 | P | | | |
| 183 | 2H3 | NC_004310 | 1344776 . . . 1343025 | BR1389 | 0.1 | P | | | |
| 184 | 2H4 | NC_004310 | 1343003 . . . 1342434 | BR1388 | 1.24 | P | | | |
| 185 | 2H5 | NC_004311 | 646508 . . . 647422 | BRA0666 | 0.22 | P | | | |
| 186 | 2H6 | NC_004310 | 1301722 . . . 1300598 | BR1340 | 0.23 | P | | | |
| 187 | 2H7 | NC_004310 | 1231195 . . . 1230170 | BR1269 | 0.15 | P | | | |
| 188 | 2H8 | NC_004311 | 739642 . . . 738779 | BRA0758 | 0.3 | P | | | |
| 189 | 2H9 | NC_004310 | 1198726 . . . 1199049 | — | 0.35 | P | | | |
| 190 | 2H10 | NC_004310 | 1181544 . . . 1181422 | BR1203 | 0.54 | P | | | |
| 191 | 2H11 | NC_004310 | 1157188 . . . 1156805 | BR1179 | 0.85 | P | | | |
| 192 | 2H12 | NC_004310 | 1125721 . . . 1127139 | BR1147 | 1.11 | P | | | |
| 193 | 3A1 | NC_004310 | 1045370 . . . 1044933 | BR1074 | 0.14 | P | | | Y |
| 194 | 3A2 | NC_004310 | 1044928 . . . 1044560 | BR1073 | 0.2 | P | | | |
| 195 | 3A3 | NC_004310 | 1037671 . . . 1038492 | BR1067 | 0.1 | P | | | |
| 196 | 3A4 | NC_004310 | 996924 . . . 997454 | BR1031 | 0.18 | P | Y | | |
| 197 | 3A5 | NC_004311 | 949569 . . . 950402 | BRA0960 | 0.4 | P | | | |
| 198 | 3A6 | NC_004310 | 940426 . . . 940698 | BR0969 | 0.1 | P | | | |
| 199 | 3A7 | NC_004310 | 880436 . . . 879642 | BR0909 | 0.48 | P | | | |
| 200 | 3A8 | NC_004310 | 878400 . . . 877045 | BR0906 | 0.34 | P | | | |
| 201 | 3A9 | NC_004310 | 875773 . . . 875895 | BR0903 | 0.1 | P | | | |
| 202 | 3A10 | NC_004311 | 1114350 . . . 1113349 | BRA1122 | 0.11 | P | | | |
| 203 | 3A11 | NC_004310 | 2048358 . . . 2049341 | BR2124 | 0.17 | P | | | |
| 204 | 3A12 | NC_004311 | 1194338 . . . 1192356 | BRA1190 | 0.5 | P | | | |
| 205 | 3B1 | NC_004310 | 1988451 . . . 1987126 | BR2062 | 0.71 | P | | | |
| | 3B2 | | | LacZ | 0.1 | P | | | |

^{a}Locus Tag entries based on published annotation; missing entries indicate that locus tags were not applicable because of alteration to the genome annotation subsequent to the original locus assignment.
^{d}WB against 6His positive = P, negative = N, showed bands of a size different than that predicted from the ORF analysis = ?.
^{c}Present in inclusion bodies = Y.
^{d}Expressed in TNT expression system = Y.
^{e}Detected by WB using IgG from rabbits immunized with rough B. melitensis WRR51 = Y.

### EXAMPLES

### Example 1

Determination of B. Suis Surface proteins, 205 genes of B. suis SP were selected and their size shown in 96 well format as follows:

### Gene Size (bp)

Gene size (bp)

| | 1 | 2 | 3 | 4 | 5 | 6 | 7 | 8 | 9 | 10 | 11 | 12 |
|---|---|---|---|---|---|---|---|---|---|---|---|---|
| A | 525 | 108 | 579 | 336 | 609 | 192 | 672 | 1,668 | 1,485 | 1,137 | 117 | 462 |
| B | 654 | 1,386 | 474 | 1,290 | 2,751 | 1,275 | 150 | 840 | 756 | 1,425 | 447 | 861 |
| C | 189 | 126 | 231 | 993 | 822 | 1,002 | 1,107 | 1,005 | 1,107 | 210 | 105 | 2,154 |
| D | 603 | 1,116 | 1,401 | 798 | 1,014 | 819 | 360 | 918 | 1,068 | 678 | 963 | 570 |
| E | 399 | 474 | 144 | 801 | 147 | 1,278 | 522 | 1,326 | 135 | 1,887 | 798 | 2,073 |
| F | 195 | 834 | 1,122 | 1,557 | 1,104 | 1,104 | 111 | 1,380 | 600 | 870 | 1,098 | 108 |
| G | 396 | 1,539 | 2,322 | 564 | 807 | 1,137 | 810 | 315 | 882 | 2,025 | 792 | 3,000 |
| H | 882 | 825 | 465 | 897 | 216 | 1,407 | 132 | 1,332 | 1,782 | 1,572 | 450 | 1,320 |
| A | 135 | 1,821 | 576 | 1,164 | 2,106 | 1,062 | 546 | 1,296 | 435 | 450 | 1,755 | 837 |
| B | 126 | 762 | 672 | 981 | 834 | 264 | 909 | 777 | 951 | 1,536 | 1,524 | 795 |
| C | 381 | 879 | 471 | 420 | 573 | 351 | 1,047 | 744 | 741 | 105 | 1,044 | 192 |
| D | 453 | 711 | 1,005 | 237 | 1,398 | 2,622 | 354 | 1,077 | 867 | 1,041 | 363 | 795 |
| E | 1017 | 237 | 813 | 969 | 900 | 384 | 1,398 | 378 | 990 | 360 | 714 | 549 |
| F | 528 | 1,563 | 906 | 537 | 789 | 1,329 | 1,599 | 1,431 | 1,758 | 1,233 | 1,287 | 1,014 |
| G | 819 | 1,125 | 1,047 | 780 | 1,377 | 1,137 | 1,047 | 504 | 597 | 1,398 | 450 | 177 |
| H | 927 | 93 | 1,752 | 570 | 915 | 1,125 | 1,026 | 864 | 324 | 123 | 384 | 1,419 |
| A | 438 | 369 | 822 | 531 | 834 | 273 | 795 | 1,356 | 123 | 1,002 | 984 | 1,983 |
| B | 1,326 | | | | | | | | | | | |
| C | | | | | | | | | | | | |
| D | | | | | | | | | | | | |
| E | | | | | | | | | | | | |
| F | | | | | | | | | | | | |
| G | | | | | | | | | | | | |
| H | | | | | | | | | | | | |

### Example 2

Purification of plasmid: Transfer 100 ul bacteria/well to a new deep plate and add 500 ul of LB culture solution with 100 ug/ml Ampicillin. Culture at 37° C. overnight for preparation of glycerol stock. The plasmids were purified using 96 turbo miniprep method (Qiagen, Inc). The 0/N cell cultures were centrifuged at 3,000 RPM for 10 min at 4° C. The supernatants were decanted and cells kept for following processing.

| | | | |
|---|---|---|---|
| Add | P1 | 250 ul | mix |
| | P2 | 250 ul | mix |
| | N3 | 350 ul | mix |

Transfer supernatants to Turbo filter and vacuum to plasmid filter. Wash the filter with 700 ul PB/well and vacuum. Wash the filter with 700 ul PE/well and vacuum. Centrifuge the filters at 3,000 RPM for 10 min at 4° C. Add 200 ul EB to each well and centrifuge the EB to a 96 well plate at 3,000 RPM for 10 min at 4° C.

### Example 3

A method of identifying and expression Brucells suis surface antigens proteins was conducted. A bioinformatics screen of the complete Brucella suis genome was made to identify genes that encode putative surface or secreted proteins. This was done by designing primers to enable PCR amplification of predicted genes with appended sequences; arraying the primers and conducting cloning reactions in a multiwell microplate format suitable for liquid handling with multichannel devices; cloning of the predicted genes into E. coli.; purifying of plasmids containing the desired sequence; verifying of correct insert size and sequence in purified plasmids; storing of plasmids and E. coli containing vector with cloned genes; (Gene bank) cloning of genes into expression vector and storage of expression plasmids and E. coli containing expression vector with cloned genes; (Protein bank); inducing of protein expression from protein bank; and verifying the expressed protein size. The results of this are presented herein. The primers used are presented herein.

### Example 4

A method of detecting antigenic proteins of Brucella suis was conducted by creating a library of Brucella suis surface antigens. We found 205 surface antigens. The antigenicity of Brucella suis proteins was diagnosed by comparing a sample from a patient or immunized or infected experimental animal to the library to determine if Brucella suis antigens were recognized. When Brucella suis was recognized, we were able to diagnose antigenicity of the proteins.

### Example 5

Diagnosing exposure to Brucella suis can be conducted by creating a library of Brucella suis surface antigens; diagnosing exposure to Brucella suis by comparing reacting a sample from a patient to with said library to determine if Brucella suis antigens are presentrecognized; and diagnosing exposure if said antigens are recognized.

Primers: The following primer sequences were used:

| | | |
|---|---|---|
| BR0042 | 525 | CACCATGAGCGAACCGAACCCAAAACGCG |
| | | GTTTTTCGAGCCGCCCTTTTTGCCC |
| BR0048 | 108 | CACCGTGAAGGGGATTTCGCTTCCACACT |
| | | GCCTTGTTGCGCGAATCCCGGATAT |
| BR0049 | 579 | CACCATGAAACTGTCGGCAAGAGCTTCAA |
| | | CCGCCTGCCGCCGAGACGCATGCGG |
| BR0050 | 336 | CACCATGCCGAAGGATGCCAATTCAGCCT |
| | | ATCTTCCTGCGGTGCCGGGGTAGGC |
| BR0057 | 609 | CACCGTGTGTCGAGGGCGGATTGGCCTTC |
| | | GACGAATTTCAAAGCGATGATTTCG |
| BR0067 | 192 | CACCGTGAAGTGGTTCAACACCACCAAGG |
| | | TGCTGCGACGAGATTGTCGGCAGAC |
| BR0087 | 570 | CACCATGATGCCCGTGATTAGACTTAACG |
| | | GGCGGACGCCGGGCCAGACCGCAAA |
| BR0105 | 672 | CACCGTGGGGGCCTTCCGCCAGGATGAGC |
| | | TCGTTCTTGGAGGATGAATGAATGA |
| BR0117 | 1668 | CACCATGTATCGTTATGATGAATTCGACC |
| | | GGCAGCACTTCTTATCTCCCCATAA |
| BR0118 | 1485 | CACCTTGCTTCCAGCGCCAGCAGGCGTCT |
| | | GGCTGCGACGGGAACAGGTTTCAGG |
| BR0119 | 1137 | CACCATGACGATGCAATGGTTCTCGCTCG |
| | | ATCCAGTGTGCGCTGGAAGCGCAAC |
| BR0139 | 117 | CACCATGTCTCCCAAAAAGCTGGAAATGC |
| | | AAGACTGATATGGCTTGTGAAAAGC |
| BR0143 | 462 | CACCATGGATCTTAGCGATCTGATTCAGC |
| | | GGCGGCGTTGGACGTCGGGTGAGCC |
| BR0149 | 654 | CACCATGTATATGGAACGCGAGACGGGCA |
| | | GTTCCGCTTGGGCGCAGCACCGCCC |
| BR0154 | 1386 | CACCTTGGTCAGAATGCGGGGGCAGGCAA |
| | | GGCCGTCGACCATACCGTGTGCACG |
| BR0202 | 474 | CACCATGCACAAGCCGAAACTGCTTCTGC |
| | | TTGCATATTGTCCTTCGCCCCGGCC |
| BR0270 | 1290 | CACCATGACTGAAACAGGTTTCAGTTTCT |
| | | AACCGCGCCCAATTCGTGCTTCGTG |
| BR0274 | 2751 | CACCTTGCCGTTTCACTGGCAGGCCCGGT |
| | | AGTCCGGCGACAAGAGCCTCGCGCT |
| BR0276 | 1275 | CACCATGAATATCCAGGCCACTTCGGCAC |
| | | AGCATGAACGGCGAGCCAGTTGGAA |
| BR0291 | 150 | CACCATGCATGAGGAAAAGCTGCCCACGC |
| | | TTCGGCTGCGATAAGCGACACTGCG |
| BR0295 | 840 | CACCATGAAATCCTGTCTGTTGAGCCTGA |
| | | ATAAAAGCTTGCCGGGAAATATCTC |
| BR0296 | 756 | CACCTTGTCGATCCTGCGCTTTGCTGCCG |
| | | ATGCAGCCGGGCTGCGCGCCCGGCT |
| BR0310 | 495 | CACCATGTCCACAAACAGCAAAACATCCG |
| | | TAAGATTTTCCCGTCCTTCACCAGA |
| BR0320 | 1425 | CACCATGAAATATCCGCGCAATCTCGTCG |
| | | ATAGAGTGTTTCGGGGAGGATGTCT |
| BR0328 | 447 | CACCATGCTGGAGAAAAGCCTCCAGCAAT |
| | | GGCGCTGCCGTGCGCGACCACATCG |
| BR0396 | 861 | CACCATGCGCTCGTGCGGGGCAGGGCAAG |
| | | GACTTTCACTGAAGCAACGGTCGCT |
| BR0437 | 189 | CACCGTGCTGGCCCGGGCCATATTCACGA |
| | | GTCCTCGGAAACGCAGGGCAGAGGA |
| BR0469 | 126 | CACCGTGAAATTCAATCGTCCGCACGCGC |
| | | ATCGGTGAAACTATTTTCATCGCGC |
| BR0475 | 231 | CACCATGTGCTCAAAGGGCAAGAAGCTTC |
| | | TGCAGCACTCGACGACTTGCTCTTC |
| BR0486 | 993 | CACCATGTCGGAACTGCTTTTTGAAACCG |
| | | GATTTGAGAGAGGTCGGGTTCAAGA |
| BR0501 | 822 | CACCATGTTTCAAAAGTTTTCAGCGGCGG |
| | | TTTCACCGTTTCCTGTTTTTTCTGG |
| BR0510 | 1002 | CACCATGCGAAGGCGCGGCGGTGTGGTTG |
| | | CGGCGGCAATCGGCGAGCGCCTGCG |
| BR0511 | 1107 | CACCGTGCAATTATGCGACCCGGCGCATA |
| | | TGGCAAACGGCGGCGAAGATCGCTC |
| BR0529 | 1005 | CACCTTGAAAGAAACCATCATGTCGATCA |
| | | GGCCCCAAAGCGGCGCTTTTCGCCA |
| BR0540 | 1107 | CACCATGCGAGTTCTTCATTTTTTTAAGA |
| | | CTGCATCAGCGACGTATAATTTTTC |
| BR0541 | 210 | CACCATGCGCCTGCACCGGCAACGCACGC |
| | | AAAAATCCCGCCATAAATTTTGTGA |
| BR0544 | 105 | CACCTTGCCCTATCGCACAGGCTTCGCTA |
| | | ATCGGTTGAAGCGAAGGTGTTGTCA |
| BR0583 | 2154 | CACCATGCGTAATACGGCAGAAAAAGATC |
| | | GAGTACCGAAACTTTTGTGCGCTCG |
| BR0605 | 603 | CACCATGACGGATCAGGGGGAGAACGAGA |
| | | GCCGCCGTTGCGGGTGCTGCCCACT |
| BR0611 | 1116 | CACCATGGAAAAAAGCCGTAAATCGCATG |
| | | CTTCCCCTGCATAAGTTTGGAAATG |
| BR0613 | 1401 | CACCATGAGCCGGTTTTCCGCACTCATGC |
| | | GCCAACCTCGCGATGCGGCAGCGGA |
| BR0615 | 798 | CACCATGAAAATCTGTACCTCTATTCTCG |
| | | GTCCAGTTCCAGCCGGCCGGAAACT |
| BR0618 | 1014 | CACCATGGCTGAAATATTTTCAGCCGGAG |
| | | TTGCGCCTGCGGGGCATTAACCTTG |
| BR0637 | 819 | CACCGTGTCTTCCTGTCGCGATTCGGACA |
| | | GTTTTTAGCGCCGACAGCCTTTGGC |
| BR0660 | 360 | CACCATGAAGAGAATCCTTCTAGCTGAAG |
| | | GGCGGCGATCAGCATTTTTTCGATT |
| BR0680 | 918 | CACCATGGCTGATCAGGCTATATCCGATA |
| | | GGCCTGCGCGCGGTTGCGCCTGTGC |
| BR0701 | 1068 | CACCTTGCAGGATAAAACAGCTCTCATCG |
| | | GCCGTAGAAATCATTGCGTCCTACC |
| BR0706 | 678 | CACCTTGTGTCATTCGCAATCGATCCGTC |
| | | AAACCGGTGGATTGAATAATCTGTG |
| BR0777 | 963 | CACCATGTCCACAATCCACTTCGATAAAA |
| | | TTCCGCTGGGTCGCCTTCCGGCGCT |
| BR0781 | 570 | CACCGTGGATGCCATAATGATGGAGCATA |
| | | GGCTGCATTGTTTGCCTGCCGGCTT |
| BR0799 | 399 | CACCATGAAAAAGCTGCTTATTTTGGCCG |
| | | CAGTTTGCGCAGTTGCGAAACTTCC |
| BR0815 | 474 | CACCTTGGGATTGCTGGAAACGGCCATTA |
| | | AGCCGCTGACCGAACGCATGAAAGG |
| BR0838 | 144 | CACCGTGCTGACAGAAGCGATTTCATGGG |
| | | TTTGCCGGGCGGGTCGGGGGGAAAC |
| BR0872 | 801 | CACCATGCGTAGTCGCAGTTTTTCATGTT |
| | | GTAACTGCCTTTTACAGCGTAATCA |
| BR0882 | 147 | CACCATGCTGGCGCGTGAACAGATGCAGC |
| | | GCGCGCCCTGCTGTGTGACCTGCAA |
| BR0888 | 1278 | CACCATGACTGTCGCAGAAGCGGTTGCCC |
| | | TTTCTTGTCTTTCAGCGTTATGAAG |
| BR0890 | 522 | CACCGTGATGAAGTCCTTATTTATTGCAT |
| | | TTCGATCACGCCGCAGGCAAAACGG |
| BR0891 | 1326 | CACCATGGGACCGATCACGGATAAGGTGG |
| | | TTCCCAGTTTTCAATGCGGATTTTT |
| BR0893 | 135 | CACCGTGGCAATTGCGCAAAACTGGCTCT |
| | | ATGCCGTGCCGGAATGCTGCCGGCA |
| BR0912 | 1887 | CACCTTGCGACGTGTGGGACAAGGCGCGC |
| | | GTTTCTTTCTTTGATCTGGGCTTCA |
| BR0916 | 798 | CACCATGACTGCCCATTCCCGACAGGACC |
| | | TAGGCCAGCGACTTTCTTCAGGTCG |
| BR0917 | 2073 | CACCTTGCAAATTTTCGTCTTCGGACGTT |
| | | TGCGGTTTTCTCCTGACGGACGGAT |
| BR0936 | 195 | CACCATGACCACATTGACCGCGAAATGGA |
| | | ANNNNNATGCACGCCCGCGCATACC |
| BR0944 | 834 | CACCTTGAACTGGCTGACGGATTACAAGA |
| | | TTTCTCACGTCCGGCGCGGAAGATA |
| BR0971 | 1122 | CACCTTGCGACGTCAGGCCGGTGCTCGTT |
| | | ATGCCTTTGCTGTCTGGGAGGACAT |
| BR0982 | 1557 | CACCATGAATAAGCTCGGCGTGTTTATCG |
| | | GTCTGCAACAGATTTAAGCAGAGAC |
| BR0990 | 1104 | CACCATGTTGATCGTCGGTGCCGGTATAT |
| | | GAAATTGCCGTCCCGCaAGGGGCCG |
| BR0999 | 1104 | CACCATGCAGCGCCTTGGCAACGGCAGCG |
| | | CGCGATGAACAAAAGGGAGGGCCCG |
| BR1001 | 111 | CACCGTGTGTTGTATGAATTTTATAAAAT |
| | | CTGACTTGACGTGATATGAAATGCC |
| BR1019 | 1095 | CACCATGCGCGTCATCCATCTCAGCGGCC |
| | | GAAATTAATTGCCGCATAAAACATT |
| BR1021 | 1380 | CACCATGTCCGCCGAAATCATTTCCCAAA |
| | | GCGGGCCGGATAACGTTCCTTCAAA |
| BR1022 | 600 | CACCATGAAATCACGGCGCATGTTCCGTC |
| | | AAGGGTTGAAATCGTCCAGCTTACG |
| BR1024 | 870 | CACCATGAGTATCAACGCGACCACGCTCG |
| | | CGGCCTGCGGATCAATGTTGGCAGG |
| BR1060 | 1098 | CACCGTGGCCGAAACCAATACCTCGCCTT |
| | | GTCGATCCCCACCATGCCGGTCAGA |
| BR1062 | 108 | CACCTTGCCGGACCGATTATGCCGGACAA |
| | | TCGTTCGCCAGCTATATGAGCGGAC |
| BR1089 | 396 | CACCATGCGCCCGCGACGGCGTGATAACG |
| | | ATTAANNNNNATGAGGGCATTGACC |
| BR1114 | 1539 | CACCATGTCCAGAGCCAGGATTTCCAACT |
| | | TTCCTGATTGATCGGCAGCGCCACG |
| BR1116 | 2322 | CACCATGCGTTGCATAGATGCAACGCTAT |
| | | AATCACCTGTCCAGCTGTGTGCTTT |
| BR1137 | 564 | CACCATGACGATGTTTCTTGTGACGCCAC |
| | | TAGGGAAATCCGCCGCCAGCTATCG |
| BR1154 | 807 | CACCGTGGAGGATGGCAGCATGACGAGGC |
| | | CACACTCCGCGGTCGAATCTCAGGG |
| BR1156 | 1137 | CACCTTGCAGGAGGCTCTGGCCCTGTTTT |
| | | GCAGGCAAAAAGGTCGTTGAAAAGT |
| BR1157 | 810 | CACCATGTCAAATCTGCAAACCCGCATCA |
| | | GAAACTAAAGAAGATTGCAGACGGA |
| BR1185 | 315 | CACCATGACGAAGCGAGTGCCCCTTACGA |
| | | AGCAGTTCCGAGGCATCGCAACCCG |
| BR1204 | 882 | CACCGTGGTAGTCATGGCCCAATCCTTGA |
| | | ACCTTTAACCGCGCCGAAAGTCAGG |
| BR1205 | 2025 | CACCTTGCCATGGCGTGCAGCCATCCACG |
| | | ATAAGGAGGAGACGAATGCAGGATT |
| BR1207 | 792 | CACCATGAGGGAGCCGACCTTGGAAACTG |
| | | TCCTGTCACGCCTACATCCGCGCCC |
| BR1213 | 3000 | CACCATGATGACTGACATGCGCATTCAGA |
| | | TGCATTCAACTTCTTACCTTCGGGA |
| BR1237 | 732 | CACCATGGCGGCGAAAGGCAGATTGTTCG |
| | | TGCGCCGTTCCATTTTTCGCCGGGC |
| BR1250 | 882 | CACCTTGCGTTCTCTTGCCCAGCGGATCG |
| | | GTAAATTGAACGTGGTTGTAGAGTA |
| BR1282 | 825 | CACCATGGTAACCTATATCGAAGCGACAA |
| | | CCGCATGGAAGGCGGGCCAAAGATA |
| BR1290 | 465 | CACCATGTACGGACGCGCCCGTTCCGGAG |
| | | CTTCTTTTTCTTTGCCGCGGCCTTT |
| BR1304 | 897 | CACCTTGACCGGCCATATCAAGATTTTCG |
| | | TGAGCCGATAATATGGCGCTTTTTC |
| BR1324 | 216 | CACCATGTTGCGCTATGTCGGCAACTGCC |
| | | TGCAACTTGCGGTAAAAACAAGCAA |
| BR1327 | 1407 | CACCATGACGACTGCCAACGATATTCTGA |
| | | TACCGAGTmATACATATCGAACTCG |
| BR1331 | 132 | CACCGTGAAGATTCCGGCGGTTGGACTGC |
| | | ATCAAACCCTGTCAATAGACGCGCG |
| BR1351 | 1332 | CACCATGGCCGCAACATTCGATACGATTT |
| | | GCGACGGGGCAGCGCTTCGAGGAAC |
| BR1364 | 1782 | CACCATGGGATGTGGTAGGGTAATCGCGG |
| | | TTCGGCAGCGGCCAGACCTGCCATG |
| BR1394 | 1572 | CACCATGGCAATTGCACCAAAGGCGGGTT |
| | | GTCGATACGGATCGTCACGAAACGC |
| BR1418 | 450 | CACCATGTGCTTGGGAAAGGGGGCTGGCT |
| | | GTCTGAGAAGGAGATCATAACATGG |
| BR1426 | 1320 | CACCATGAGTATTCTTGGTGGTCATGGAG |
| | | AAAACTCTCTCTCAGCCATTGGCCG |
| BR1434 | 135 | CACCATGGCGGCAGACGCGCCACATGATA |
| | | TTTTAACAGAATCGCCGGAACCGCT |
| BR1437 | 1821 | CACCATGCGGCTGAGATTGTTTTCGGCAA |
| | | ATCGTATTCGTTTGAAACCATTGCT |
| BR1447 | 576 | CACCGTGGCGCGTCCGCCCAATCTTGCGA |
| | | CGCTTCCCGGAAACGATAACCGACG |
| BR1488 | 1164 | CACCATGTTCTTCGCGGTGCTGGGCGCTG |
| | | TAACCGGCCTCCGGCATTGTTGCGG |
| BR1498 | 255 | CACCTTGAGCGTAAAACTCGTCTATTTCG |
| | | GCCCCCGGTCATTGGAGGGAACAGC |
| BR1501 | 2106 | CACCATGGGAAACGCCCGACATTGCAGGA |
| | | TTTGTCCCGGAAGTGGTCGTGGATC |
| BR1503 | 1062 | CACCGTGACTTTATCAGGGCAAGTTCCGG |
| | | TCTTCCTGCGAAAACCTGCTCATAA |
| BR1507 | 546 | CACCATGGCCGCGCATTATGCAGGCATTC |
| | | GCCGCGCGGCAGAAGCTCGACCAGC |
| BR1522 | 1296 | CACCATGCCCAAGGGGCTTTATGCGCGCT |
| | | AGCCGGAATATGAATGGTGGCGCGC |
| BR1531 | 435 | CACCATGTTCTCAGGACATGGCATGGCCG |
| | | ACCGATACGGGAATACACGTGCAGG |
| BR1532 | 450 | CACCTTGCTCTGGATCACGCTCGTCAGCG |
| | | TTTTGTGTAAAACAAAAAAGGGTGC |
| BR1620 | 1755 | CACCGTGAAGCGTATCAGGAACCGGAAAC |
| | | AAAGCCCCCGTAGAGGCTGACCTGA |
| BR1622 | 837 | CACCATGTCGAAAGCACGCACTTCACCCG |
| | | TTTTACCGCTCCAGCCGTAATGCCA |
| BR1635 | 126 | CACCTTGCTTGGCTTTCTGTTTGATGGCG |
| | | AAACAGGAACCGCTCTTCTAGGGGA |
| BR1636 | 726 | CACCATGAGCCCCCACGACAGCAACATCA |
| | | CAAACCCAAATATGCTTTTTGCATC |
| BR1671 | 672 | CACCGTGGAGCGACCCTACCAGTCGCGCA |
| | | CGGCGCGACGATCGGCTGTGCCTTC |
| BR1679 | 981 | CACCATGTTCAGCCGATTCTTCCGTAATG |
| | | GGCGCTGCCGATCAATATGCCCGCC |
| BR1689 | 834 | CACCTTGCAGGGTAAAACTGACGTGATAT |
| | | GAAGGAGACGGGACGCTTCTGCTTG |
| BR1695 | 264 | CACCGTGAACGAGGCTGATGCGCTTGATA |
| | | AAAACCCTTTTCGATGCGGGAATAG |
| BR1714 | 909 | CACCTTGCGCAAGCCGTACAGTCCATCGA |
| | | TAGCTTTTCCGCACCCGTCAAAAGC |
| BR1755 | 777 | CACCGTGGGGCCGCTTGCAACCGGCTTTT |
| | | TCCTCGTCGGGCGATCAATATGCCG |
| BR1806 | 951 | CACCTTGCTGGCTTACATATCCGGGCGGT |
| | | TCGGCTGCCTCCCGTCGAAAGGCGT |
| BR1826 | 1536 | CACCTTGATGTCCGAATATCTTTTGGAAA |
| | | GGCCGCGTTGGTTGCAGGCTTGCCA |
| BR1866 | 1524 | CACCTTGTCTCCTGCGCTTTCTTCTGCCC |
| | | TCGCAAAGTTGCCTTTTCCTTCTCA |
| BR1869 | 795 | CACCATGGCAGGAGCGGGCATGAAACCCA |
| | | TTGACCGTGTGAAGATTGCTGGTCT |
| BR1870 | 381 | CACCATGGCGCTGGAACCGGCTGCTGCTG |
| | | GATTACGTCGCGGATGCGCTCCACG |
| BR1873 | 879 | CACCATGAAACTGATGGAATCCGCGCCTT |
| | | TCCTTTCTGCGCGCGCGTGGTCATG |
| BR1893 | 471 | CACCGTGAAGGCGATTTTTCAAACGGCGC |
| | | CTGCATCCCCACCTGAATACGACCA |
| BR1930 | 420 | CACCATGCCAATTCTTTACACGACCCAGT |
| | | GGCTACGCTCAGGCGAACGTCGAGA |
| BR1934 | 573 | CACCATGTCCGTCGTCCTGCCGCCGGTCA |
| | | CCCTACCACAAAACAGGCAAGCCGC |
| BR1937 | 351 | CACCATGGCTGTCCGCCGCATCGTTGCCA |
| | | ATGCTTGTGGGTCAGAATATTCACG |
| BR1945 | 1047 | CACCATGACGCAAAACGAACGTATAGGCG |
| | | ACCCGATACCAAAACCCTGCTGATG |
| BR1948 | 744 | CACCATGCTGATTGTCGAGTCGCATCGCG |
| | | GTTCTTGTCCGGGTTACGATATTGC |
| BR1982 | 741 | CACCATGGCAACACCGTTCCGCAATTGCA |
| | | ATCCTTGCGGAACAGAATGCGCCCG |
| BR2001 | 183 | CACCATGGCTGAGCAGGCTGCCCGCAATG |
| | | AAGCATCTCGCATGAATATGGAAAA |
| BR2002 | 105 | CACCTTGCCGATTGTGCTGGCAAGCACGA |
| | | AAATGCGTCGCGTTTTTCCATATTC |
| BR2009 | 1044 | CACCATGACAGGAGAGGACATGCAAGCGA |
| | | GCGGCGCATGCGCCAGCGGCGGCCC |
| BR2047 | 192 | CACCATGGCGCGGCTCTTAGCACAGAATA |
| | | TTCCCTGAAGGGCATTTCCGCGGCA |
| BR2059 | 453 | CACCTTGCCAATTGAGACGATCTTGCGTT |
| | | TCGATATCCTGCCCGTCGCCGCACG |
| BR2072 | 711 | CACCGTGCTCGAAAACCGATATGCTCATC |
| | | AGTTCGATATCGTATTGCGCGGACG |
| BR2088 | 1005 | CACCATGCGCTCCTCCGGCAGATCGAAAT |
| | | CCCCGATCTCCCGGCGGAATTTTCC |
| BR2091 | 237 | CACCATGGCGCAAACCATCGGCCTTTTTC |
| | | TCCATGATGGTCGCAATGGCTGCCT |
| BR2097 | 1398 | CACCATGACCGCAAGCCAAGATTTTGTTG |
| | | ATACCTGTAGTGTTCCGACTTGAAC |
| BR2134 | 2622 | CACCGTGCGCCCCGAGCATGTCCGCTCAC |
| | | GAAATCATCATCGCCCGTGGGCACC |
| BR2135 | 354 | CACCATGAACTTCAAGACCCGTATGGCTA |
| | | AAGTGGCTTGAAGCAGACTTTGGTG |
| BR2145 | 1077 | CACCATGCGCGTTCTTGGAATAGAAACAA |
| | | TGCCTTTGCTCCCCGCCGTCCCGTG |
| BR2146 | 867 | CACCATGCAGACTGTGCTCAGCATTGCTG |
| | | TTTGCGGGCCAGCCTTCCTGCAAGG |
| BR2148 | 1041 | CACCATGCTACTGCCACCCATCAAAGAAA |
| | | TATTGGCGCATGTTCAGCAAAAGTG |
| BR2175 | 363 | CACCATGGCAAATTATTCCGTTACTTCCG |
| | | AAGATGCTGGTTGAAGCCGCCAGGC |
| BRA0004 | 795 | CACCATGAACAAGATGTCCAGGATCATAA |
| | | GGCAGCCTGTAATTGCCGCTCGCGC |
| BRA0018 | 1017 | CACCGTGGCCACCGGCAAGGTTTATGAAC |
| | | GTTGATGGCCGTTGTCTTTACGCCT |
| BRA0020 | 237 | CACCATGACCATCAAGCACAACATCATCG |
| | | CTGACCGCTCTTCATTGCGTCACCT |
| BRA0037 | 813 | CACCATGACAGATGTCTTGCTCCTGCTGA |
| | | TTCCACTTTTTCAAACCGGGTGGCT |
| BRA0058 | 969 | CACCATGATAAGATCTGTCGTACGGGGTA |
| | | CCAGCGCACAAGAACCGCGCCCCAG |
| BRA0059 | 900 | CACCATGAGTTTTTTTCCCGCACGAACCG |
| | | TTCGCGCATATCGCCGCCATGAGAA |
| BRA0063 | 384 | CACCTTGTGTAACGTCCTGTCACGTAAGG |
| | | CTTCGAGGTTTTACCCTGACGCTTC |
| BRA0076 | 1398 | CACCGTGGAGATAGGTCTAACCCAAATTG |
| | | ATCGGTTTCGACCGCAAGCTCTATG |
| BRA0077 | 378 | CACCATGAAACGCTTCCGCATCGTTGCCC |
| | | GCCGGCGTTGCGGCGGGTGAGCGAG |
| BRA0107 | 990 | CACCATGTCGGCTCTACCTGATCATATTG |
| | | GTTCTTGTCCGGGTTACGATATTGC |
| BRA0117 | 1389 | CACCATGGGTTTCATCAAGAAAATGTTCT |
| | | GGCAACTCCTGCGATGGCGCGGGCA |
| BRA0135 | 360 | CACCATGATCGCTGTCATTTTCGAAGCTT |
| | | TACATGATCGATGTCCTCCTTATGG |
| BRA0158 | 714 | CACCATGAACAAAGCGATCCTTGCAGTAG |
| | | GAAAGGAGAAAACTGGTCGAGAATC |
| BRA0203 | 549 | CACCTTGATGGAGAGTGGCATGTCCAAGC |
| | | GGCCGCCAGATGCCGGAAAGTCGCA |
| BRA0228 | 528 | CACCATGGCATTTGTGCATTTTTCGAATC |
| | | GGTCTGCCCAGGTTTGCCGTGCCGC |
| BRA0236 | 1563 | CACCATGGCAAAAGCAGCGACCCCGAAAA |
| | | GGCAGCTTCAGCCGCCAGCTTCTTG |
| BRA0243 | 906 | CACCATGGAATATTTCTTCCAGCAGGTGA |
| | | CACCTTTTCGACTTCAGGTCGGCCA |
| BRA0279 | 537 | CACCATGAAACGCGCTCTGTTTCTTGCCC |
| | | ATGCATATGGCTTTCGCCGTCCGGT |
| BRA0322 | 789 | CACCATGGCCCGCTCAGCCCTTCTCAACG |
| | | GATCGGCTTCTTGAGTGTCTTCAGG |
| BRA0335 | 1329 | CACCATGAAAATCGGCATCATCAATACAA |
| | | TTCGAGCAGCGGCGACCAGGCCGGG |
| BRA0350 | 1599 | CACCATGGCACCTCGCGTTCTCGTATCCG |
| | | AACTTCCCCGACGTTGAACTCAAGC |
| BRA0384 | 1431 | CACCATGGTTTCTGTCCCGGCCTCGCGCC |
| | | CCAGATCCAGAACAACAATACCAAA |
| BRA0416 | 1758 | CACCTTGGTGAAGCGTATCAGGAACCGGA |
| | | AAAGCCCCCGTAGAGGCTGACCTGA |
| BRA0421 | 1233 | CACCATGGATGGTTCCGTGGTCGTCAGCC |
| | | TGCGCGTTGCCTCCTGTCGTGACGG |
| BRA0422 | 1287 | CACCATGACAGATTTTTACAGCCTCATCC |
| | | CTCGGCAGCCGGCTTGAACTGTGCG |
| BRA0423 | 1014 | CACCGTGGTCAATATCTATAACTGGTCGG |
| | | CTGGCCGGTGACGATCTTGGTCCAG |
| BRA0427 | 819 | CACCATGCAGAACAACTGGTCCCGCTTCA |
| | | TCCCATACGGAATGCTGCCTGTTCG |
| BRA0428 | 1125 | CACCATGACAAGTCTTTCCTGGATCACGC |
| | | TGTCGTGGTCCCCGGAAAGGTGATG |
| BRA0429 | 1047 | CACCATGAACAGGCACCGCAAAGGGTTCA |
| | | CGATTTGCGCCCAGTGCCATCAACA |
| BRA0430 | 780 | CACCATGAAGTGTTATCTTATTAATCTCG |
| | | CGCAATATCCCGATCAAATGGAATG |
| BRA0432 | 1377 | CACCATGCGGGGGCAGGCAATTGCGATAA |
| | | GGCCGTCGACCATACCGTGTGCACG |
| BRA0463 | 1137 | CACCATGGATCACCACGCGGGCATTCCCG |
| | | ATCCTTTGTTTTGACGCGCATCTTT |
| BRA0472 | 1047 | CACCATGTCCGTATCCAAGAAATATATCC |
| | | TTTATCCGGCGTGGAGGCAGTATCG |
| BRA0473 | 504 | CACCATGGTGACGGCCAGAATGCGCGATG |
| | | AACGTAGAGCCTTTCATAAAGTCCG |
| BRA0497 | 597 | CACCATGAACAATGCTCTCCTCGATTCCA |
| | | GAAGAACACCTTGAAGCGTTCAGCG |
| BRA0529 | 1398 | CACCATGGCCGCGGCCATGCTTCTGCCCG |
| | | ACGCACCCAGAATGAGCGTGTGAAG |
| BRA0544 | 450 | CACCATGCTTCGCCAGGAGATTTCCCGGG |
| | | TTGCAGCAGCGATTTTACAAGTGCG |
| BRA0567 | 177 | CACCTTGCAGTCCGTCCTGCGGGAGGGAG |
| | | GGCCAAATGCGGCGCAGAAGCAAAC |
| BRA0590 | 927 | CACCATGCCGAAAACGACGGGGGGTGGCG |
| | | TGAGATCCGTCCTGTTTTTTTCTTG |
| BRA0594 | 1020 | CACCATGTTTTTCCCGATCAAGCTTCTCG |
| | | CCAAAAAATACAAACAATGGCCCTT |
| BRA0618 | 93 | CACCATGGCGTCAATCGAAATCTTGATCA |
| | | TTTTCCCCGAAAACCTGTCTTCTGC |
| BRA0631 | 1752 | CACCATGACCGGCGCAGAAATGGTTATCC |
| | | GACGAGCGCGCGGCCCTTGGCATCA |
| BRA0632 | 570 | CACCATGAACGCACAAAACCTAGCTTCCG |
| | | CATGCCTTCCGCACCACGGTTCATG |
| BRA0666 | 915 | CACCATGCTTTACGGTATTTTACTTGCGT |
| | | GAAATACCCCGCTTTTTCTTGGACC |
| BRA0675 | 1125 | CACCATGAGCGAACCCAATTCCGAAACGA |
| | | ACGCTCGCCCGTGGCGCGCTTGCGG |
| BRA0732 | 1317 | CACCATGAGTGATGCATTGCGGATCGGTG |
| | | GCCAGCGCGTTCAATGCGGATCATC |
| BRA0737 | 1026 | CACCATGACGGGTTCCGGAAACCGCCTAA |
| | | TTCCGTGCCTTGAATAGCGGCGAGT |
| BRA0758 | 864 | CACCATGAAAAAGCTCCTTGCACATTCCG |
| | | ATCCACCACCGAAACGCTCACCGTG |
| BRA0763 | 306 | CACCATGAACGGTCAAAACATCCGCATTC |
| | | AAGCTTGATCTCGACATCGACACCG |
| BRA0767 | 324 | CACCGTGATCGGATTAAAATGCTTGAGTG |
| | | CGACGTGATCGTCAGCCCGGTCATC |
| BRA0791 | 123 | CACCATGCTCTATTGCGAAACATCCAATG |
| | | TCCACGAACTTCTGCAATCTGCACG |
| BRA0816 | 384 | CACCGTGCGCGAATATTATACGATAACGG |
| | | GGTCGATACGCCAAGCTCCGCCAGA |
| BRA0853 | 1419 | CACCATGTCGAAACCGGTTATTACCCGTT |
| | | ACCGATTTGATCGCCGATGCGGGCG |
| BRA0915 | 438 | CACCATGATCGACGATATCTACAATAAAC |
| | | GGCGGCTTTCGCCTTTTCCTCGATC |
| BRA0916 | 369 | CACCATGGGGTCTTGCGGCGGGAAACATA |
| | | TTTTCCGGTGCGCGAAGCCGAGCAG |
| BRA0921 | 822 | CACCATGAAATCATCCCGCAATATTGATC |
| | | CAGGCCTTTTTTTCTTCGCTTCGCTC |
| BRA0948 | 531 | CACCGTGAGCACTCCGCCGAAACCATTGC |
| | | TTCCAACCACCAGCCCGCATCCGTG |
| BRA0960 | 834 | CACCATGTCGTCAGTCCTTTCCCGCTATG |
| | | ATTCCAGGCCGGGATGCCGGTGCCC |
| BRA0999 | 273 | CACCATGACGCCGGAGACTGCATTGATCA |
| | | TGCTGCCTGTTTGTGCGCTGCGCTG |
| BRA1033 | 3438 | CACCTTGGCAGAGAAACCACAAAAAATTC |
| | | TTGGAACTCGAAGATGGAACGGAAA |
| BRA1047 | 795 | CACCATGAAAAATGCGGCTTCCATTACGA |
| | | CTTCTTTGCAGCGTCCCGGGCGGCC |
| BRA1050 | 1356 | CACCATGTTTCAAAAGATACTCATAGCCA |
| | | GGCGCTTTCGTTCTTGCTCTTGTTG |
| BRA1053 | 123 | CACCTTGGGTTTTTTCCTGAAAAGCCTTG |
| | | AATAAAGCCGGAATAATTCCTAAGC |
| BRA1080 | 1206 | CACCATGTCGCTGGAAGGAATAGGCTTCG |
| | | AGCTAAATCGGGGGTTCGGTCAAAC |
| BRA1099 | 1179 | CACCATGCAGAACAACAAAGTCGTCATAA |
| | | TGCTCTCACCAGCTTTGCTGCGCCT |
| BRA1105 | 564 | CACCATGAAAGTCAAAGACGCAGATATTC |
| | | ATGCTGTTTGGGAGCCTGAAGCTGG |
| BRA1106 | 765 | CACCATGTCAATGTCGTTGTCTTCGCGCC |
| | | TCCGCGAACAGCTTTCACCAGCGTT |
| BRA1122 | 1002 | CACCATGAAAAATCTGCATTCCTTGTTTC |
| | | TTTCGACAGGCAGTCTTTCAGCGAA |
| BRA1143 | 1137 | CACCTTGCGCCCCTTGCTCGTGTTGATGG |
| | | GGCTGGCAGTTGCGGCGCATCCGCC |
| BRA1144 | 984 | CACCATGTTTCGATGGGGTGTACTTTCCA |
| | | CACAGTCTCGCTCACCCCGCTTCTG |
| BRA1190 | 1983 | CACCATGAAGTTCACGAGAACGCTGGTGC |
| | | GAACTTGAAGGCCGTCTGGAAAGTG |
| BRA1202 | 1326 | CACCATGAGCGAGATCGGTTCTTATCACG |
| | | CTTCCCGATGCAGAATTGCGAGAAA |

SEQUENCE LISTING: The complete nucleotide sequences of the putative surface and secreted protein genes amplified by PCR from B. suis DNA are listed below.

>BR0529
TTGAAAGAAACCATCATGTCGATCACCCGACGCCGTGTCCTTGTCGGTGGCGCTGTCGCCGCAACGTTTTC
CATGCTTCCCCGGTTCGCCTTTGCGCAAGGCGAGCAGCAACCGGTCATCTGGCAGGGGCAGGCCTTGGGCG
CTCCGGCCAAGATCATCTTCTATCATCCCGGCCGCAAGGTGGCGGAGCGGCTTGTCGGTGAAAGCGCGCGG
GAGGCGGAAAGGCTTGAGAATATCTTCAGCCTCTATCGCCCGGATTCCGAAATTGTGCGGCTGAACCATAC
GGGCGCATTGGCTTCGCCTGCGCCGGAAATGGTGGAATTGCTTGGCATTTGCCGCACCTGCTGGCAACTCA
GCGACGGGCTGTTCGATCCAACTGTTCAGCCCTTGTGGCAATGCCTGAGAAAGCACTTCTCGCAGGCAAAT
CCCGCACCTGAAGGCCCTTCGCGGGCGGAGTGGGATGCGGCGCTTGCAAAGATAGGCTTCGATCATGTGCT
CTTCAACGCGGAGCGCATCGCCTTTACCAAGCCCGGAATGGCGCTGACACTGAACGGCATTGCGCAGGGCT
ATGTGACTGATCGCGTCGTGGGCCTTCTGCGAGATGCAGGCGTGAGCGAGGCACTTGTCGATATGGGCGAA
TATCGCGCTATCGGCGGGCAGCCGGATGGCAAGCCCTGGCGTATCGGCATCGCCGATCTTGAGGCGGGGGC
CGCGCCGGAAGAATTTATCGACATCATGGATCAGGCGCTTGCCACCTCCAGCTTTGCGGGTTTCCAGTTTG
AGGAAAGCGGGCGTTTCAACCATCTGCTCAATCCGAAAACCGGCTATTCGGCGGCCCTTTACCGGCGTGTG
ACGGTGGTTGCGCCAGATACGGCGATGGCCGATGCGCTTGCTACGGCTTTCAGCCTGATGGAACCGGCACA
AATAAAGGAAACGCTTAAAAAACTGCCCGGCATTTTCGCTTATACCGTCAGCCGCGATGGCGAAAAGCGCC
GCTTTGGGGCC
>BR0511
GTGCAATTATGCGACCCGGCGCATATGCCGGACGTGGATTTTGTTGATCTCAGCGCCTTTCCCGATCCGCG
GGCAGAGGCTGGGCGGCGTATGGAAACCGATCTGTCCGCACCGCTTGATCTGCGCCGGGACAGGATGTCCG
CCCATGCGCTTTATCGCATCGGCGCGCGCCAATATATCTGGTATATCCGCGCCCATCACATCATTATCGAC
GGTTACGGGCTGGCGCTGATCGAACAGCGTTGCGGCCAGCTTTACAGCCATTTTTGCGGCCAGGGCGAAAG
TGGCCACCCCTTCCATCCTTTTGCAAGCTTCATTCATGAGGAGGACGCCTATCGGGCAAGCCCCCGCTGGG
AGGCCGACCGCAAATTCTGGCAGGATTATCTGGACGGGCCTGTGCGCCTTCCCATCCTCGATCGGGGCGCG
GAAGATTATGGCGAGGCGGGCTATCATCATGACGTCGCCTTTCGCGACGATATTTCCATAGGCCTTCGCGC
GATGGCAAAAGCCACGGATATCGGCTGGCCCGATCTTCTGGTGCTTTTATCGGGGCTCTATCTCTCGCGGA
TGCTTCCGATGCAGGGAGAGGACGAGGGGCTGACGCTGTGGCTGCCTTTCATGAGCCGCTGGGGCAGCGTG
GGCGCACATATGCCGGGGCTTCTGGTCAATATCCTGCCGTTCCATCTTGAATTCGATTCCGGCGAAACATT
GTTCTCTGCGTTACGGCGCAATATGGACATGCTGAAAAAGCACCGGCTGCACGGGCGCTATCGCATCGAAC
AGATTTCCGCCGACCGCGGCCTCGTCAGTGGAAGCCGCTTTTTCTTCTCGCCGCTGATTAATGTGCTGCCA
TTCAGCACGCCGCAATTTGCAGGTTGCCGGGTTGCGCGTGAGGTTCTGGCCAATGGCCCCGGCGACGGCTT
CAACCTGACCTATCGTGCCGATGTCGATGGCGCTGGCCTTACCTTGAAAATCGAAGCGGCATCGAACATGA
TGGATGCGCAAACCTTTGCCCGCATTGCGGCGGGCCTTCCGGCCTTTCTCAAGGCAAGCCTTGCGCCCCAT
CTCATCGAGCGTCCGGTGAGCGATCTTCGCCGCCGTTTGCCA
>BR0510
ATGCGAAGGCGCGGCGGTGTGGTTGCGCTCACGCACCACCTGATAGCCCGGACGCCAGATATAACGCACCG
GCGCACTCAGCATATGCACAAGACGGGTGAACGGGAACAGGATGAAGATCGTGAGCCCGAGGAAGATGTGC
ATCTTGAACATGATGGAAGCATCGGCAACATAGCTGGAGGCGGCCGGGTTGAAGGTGAAGATGCCCTGCGC
CCAGTTCATGAACTTGACCATTTCGCCGCCATCGATGTGCTGGAGCGAAACCGGGATCGAACTCAGGCCGA
GCAGAAGCTGCACCCACAACAAGATGATGATGAGTGTGTCGCTTGGGCGCGAGGAAGCGCGCACGCGCGGG
TCGAACAGGCGGCGATGCACCAGCATGCTGGCGCCGATGATTGCCATCAGGCCGGCAATGCCGCCCGCGAC
GATGGCGAGGATCTGCTTTGCCTCATGGCTGATGCCGAGCGTGTCGAAGATCCAGATCGGGGTCAGAAGGC
CCACAAAGTGGCCTGCGAAAATGACGAGAACGCCGAGATGGAACAGGACCGAGCCCCACATGAGCTGCCTG
CGGCGCAGAAGCTGGCTGGAGCCGGAGCGCCAGGTATAGGGTTCGCGGTCGTAGCGGACGATCGAGCCGAG
TGCGAGGACGCTCAGCGCGATATAGGGATAGATGCCGAACAGAAACGTGTTGAAATAGCTGGCCATCTTCC
GTCTCCTAGATTTGTGTTGATGGCGAAGCGGGCTTGCGGTGTGCGGCGCGCATCTGCATGCGCAGCCGATC
CGGCCCGCAGGCATTTTCGCCTGCATTGGCGCCGAAAGTCACAGCCTCTTCTTCCCAGATGCGGTCGAGTG
CGGCGAGGTCGTCCGGGTCGTCTTCTTCCTGGCGAAGCAATTCTCCGACGAGTTTCTCGTCCGGCTTGGCA
TTGGCAAGATGAAGAAGCGCGGCGAAGGCCCCGGCATAGGGCGACTGGCGCTTGCGCAGGCGCTCGCCGAT
TGCCGCCG
>BR0501
ATGTTTCAAAAGTTTTCAGCGGCGGGTGCCGCGCTATTATTCACCGTCGGCATTGCCTGTGCCAATGACAT
GGCATCAAAGGAGCATGGCTCTGAAACGGTCACGATTGTCGAGCCGCAACTGCATATTAAGCCGGGCGGCG
CGGCGGCACCGCAAGTGGCGCTGACGCTTGATGCCTGCATGGGCAAGACAGATCATCGCATTCTGGACGTG
CTGGTGAAAGACCGCATTCCGGCTACGATTTTCGTGACAGGGCGATGGATAAAGCAAAATGCCGAAGCCGT
GGCGGTTCTCAAGGCTCATCCCGATCTCTTCGATGTAGAGAACCATGGCGCGATGCATGTGCCAGCCATCA
CGAACATGCCGCTGATGTATGGCATCAAGACCGCTGGCTCTCTCGATGCCGTGCGCAAGGAAATCGAAGGC
GGTGCGGAAGCGATTACCGCCGAGGGCATGCCAAAGCCGCTTTGATATCGCGATGCAACGGCGCGCTATTC
CACCGATGCGGTGCAGCTTGCCACCAGCATGGGCTACAAGATCGCCGGTTATTCGCTGAACGGTGACATGG
GCGCGAGCTTGCCCGCAGAGACTGTGGAACGGCGCATCGCCGGTGCGCATGACGGCGATGTCATTATTTCG
CATATCAACCAGCCGACCCGTTCTTCGGGCGAGGGGGTAGCGAAAGGCATTCTGGCTTTGAAGGCCAAGGG
CATGAAATTCGTGCGCCTGAAGGATGTGCAAACCACGATGGAACTCAACCCCGTCCCGGAACACGATCTTC
CTGTCAACACCGCTGCCCAGAAAAAACAGGAAACGGTGAAA
>BR0486
ATGTCGGAACTGCTTTTTGAAACCGATGCAAATCCGATCCCGAGCCGAATAAGAGGCGGTCTTTTCCATGC
GCCGGACGGGAAGGCCCTGCGCTACGCCCTGCTGAAAGCCGAAAGCCGGCCGTGCCGGGGCACGGTCATCG
TGCTTCAGGGCCGCAACGAATTCATCGAAAATTATTATGAAACCATGTCCGACCTCGCCGGGCGCGGTTTT
ACCGTTGCCACCTTCGACTGGCGCGGCCAGGGCGGCTCGCATCGCCTGCTGCGCGACCGCCTGCGCGGCTA
TGTGCGCAGCTTCAACGATTATGCCGACGATCTCGACCATTTCCTGACCGGAATCGTGCTGCCCGACTGCC
CGCCGCCATTTTTCATTCTCGCTCACTCGGCAGGTGCGCTGGTCGCGCTTTCCTCACTGGAACGGCTCTCC
TCCCGCATCACCCGCATGGTGCTTTGCGCGCCCCTGATGGGGCTTGGCGGGCAGAAAATCAGCGATGACAA
TGTGCGCCGCATTACCGCCGCGCTGCGCTGGATCGGCCTGGGGAGAATCTATGCGGCAGGCGGGCGCACCC
TGTCGGCGGCGCGCGCCTTTGCCGACAATCCACTGACCAGTGATCCGTTACGGTTCATGCGCAATGTGGAA
ATCACACGGACTTATACGGACCTCGCGCTTGGCGGCCCCACCGTGCGCTGGGTCTGGAGCGCACTGGAAAC
CGCATGGCGCATCAACCAGCCGGATTTTTACAAAAGCCCCATCGCGCCGGTTCTGATTATCGCGGCTGGTG
CTGACCGCGTTGTTTCAACAGCGGTGATCGAGCGTTTCGTGGCGCGCACGCGAAACATCTCGCTCGCCGTT
ATTGACGGTGCGCGCCACGAAATGCTCCAGGAAGCAGATTTCTACCGCGAACAGGTGCTGGCGGCTTTCGA
CGCCTTCATCCCCGGCTCATCGCCGGTGGAATCCATGCCCCAAAGTCTTGAACCCGACCTCTCTCAAATC
>BR0475
ATGTGCTCAAAGGGCAAGAAGCTTCACCAAAATATCCGCGCCCGCCGGCAAGGGATGAAAGCCGTGAAGCT
TTACCCCCGCCGATCAAAGATGACGCTGGAACGCGCCCTGTCGTGGATTCAGGATGACGAACTGGTGGAAG
TGACGCCGAAGTCGATCCGTCTTCGCAAGCTTTATCTCGATCCCAATGAGCACAAGCGTTTTCAGAAGAGC
AAGTCGTCGAGTGCTGCA
>BR0469
GTGAAATTCAATCGTCCGCACGCGCTGATTCAAAACTATATCAATGAAGTATGGCAAAGTGCGGCAATGAA
GATGCGAACGAACGATCCGCCGAAGCCCCTGCGCGATGAAAATAGTTTCACCGAT
>BR0437
GTGCTGGCCCGGGCCATATTCACGACGGTTGACCGGGGACTTCGGACGGCCCCAGATGTTTTCGCCAAGAC
GGCGGTCAATCTTATACTTTGCGGATTCGCGCTTGCTCATCGCATTTCCTTCAAAAAGTTAGAGCGCCCGG
AGGCGCTGGAGGAAACGCGCCCTCCTCTGCCCTGCGTTTCCGAGGAC
>BR0396
ATGCGCTCGTGCGGGGCAGGGCAAGCACGGCCCTTTGGCCCACACAGCTTTTCCTCCCACATTCCTGTCAC
AGCTCGTATTTCGGCGGCGGAGCATTTGTATTGCCACTTTTGCGTTCCTATGTGCGAAAGGACAAAACCAG
ACAGGAGATTTCGGATGTTCGATGCCAAAAAATTTCTCGATCAGTTTCTCGGATCACAGGTGCCGGGTATG
TCGGGAAGTGTCCGCGACAAGGCTGGACAGGTAACAGATATGGCGAAGAAGAACCCGCTTGCGACGGGTGC
CATAGCCGCAGCCATTTTTGGCACGAAAACGGGCCGCAAACTTGCTGGCAATGTAGCAACTGTCGGCGGCA
TCGCCGCCATAGCCGGATTGGGCTATCTTGCCTACAAGAATTATAAGTCCGGTCAGGCCCCGCAGGAGGCG
GAACAGCCGGTGGCAAAAGAGCCGGAGCTTCTTCCGGCGCCACCGGCCGATTCACCCTTCCATCCGCATTC
TCCCGCGATGAGCAATAGTTTCGCGCTGACGCTGGTTCAGGCCATGATCGCCGCCGCCAGGGCTGATGGTT
ATATTGATGATGCCGAACGTGCCCGCATCATGGAAAAGGTGAAGATTTCCGGTCTGGACGACGAGGCTGAG
GCCTTCCTTGCAAGGGAACTGGCCGAACCCGTCGATCTCGATGTGCTGGTTGCCGCAGCGCAGACGGAAGA
GCAGAAAGTTGAACTTTATACGGCATCGCGGCTGGCTATCGATCCAGATAACCGCGCCGAACGCGGCTATC
TGGATATGCTTGCCGGGCGGCTTGGCCTGCAAGATGCGCTGGTGGACCACATCGAAGCGACCGTTGCTTCA
GTGAAAGTC
>BR0328
ATGCTGGAGAAAAGCCTCCAGCAATGCTCATTCGCGCGCGGTAAGGCAAAGTGCGCGCCCGTCGCGCATGA
CGGACTTGGCTGCAAGGTCGATCTGCAAGCCGCCGATTTCGATCAGGGGATTGGGATTGCCGCTATAGCGC
CGCGCTACCGCGTTCAGCCGTGCGGAAAGATCGAAGGGCTTCGTCAGATAATCGTCGGCGCCTGCGTTCAG
TCCCTGAATGCGGTCGGAAATCTGGTCCAGGGCCGTCAATATGATGACAGGCGTGGAACAGCCCTTTGTGC
GCAGATCACGCAAGAAGGACAGGCCTTGCCCGTCCGGCAGCATCAAATCCAGAAGAACGAGATCGTAGCGC
GCGCTCTCCATGTAGTCATGGGCGGTGTCGAGACGGCTGACCCAATCAACGCAATGGCCTTCAGCCGCGAT
GTGGTCGCGCACGGCAGCGCC
>BR0320
ATGAAATATCCGCGCAATCTCGTCGGCTATGGACGCAACACGCCAGACCCGCGCTGGCCGGGAGGGGCAAA
TATCGCCGTGCAGTTTGTCGTCAATTATGAGGAAGGCGGCGAATGCTGCATTCTCGATGGCGACCCGGCTT
CCGAATGCCTGCTTTCCGAGATTGTCGGCGCGCAGCCCTGGCGGGGCCAGCGCAATCTCAACATGGAATCC
ATTTATGAATATGGCGCCCGTTCCGGCTTCTGGCGCCTGTGGCGCGCCTTTACCCGCCGCAATATGCCCGT
CACCGTCTATGGGGTGACGCTTGCCATGGCGCGCAACCCGGAAGCCGTGGCCGCCATGAAAGAAGCCGGCT
GGGAAATCGCCAGCCATGGCCTGCGATGGTGGGAATACAAAGACGTGCCGGAGGAGATCGAGCGCGAACAT
ATCCGCGAGGCCGTGCGCCTCCACACGGAAGTTACCGGCTCGCGCCCGCTCGGCATCTATCAGGGCAAGCC
CTCCGACAACACGCTGAAGCTGGTGATGGAGGAAGGCGGCTTCCTCTATTCCGCCGATTCCTATGCCGACG
AACTGCCCTATTGGGTAAAGGGCCCGAAAGGCCCGCACCTGATCGTGCCCTATACGCTGGACGCAAACGAC
ATGCGTTTCGCCACGCCACAAGGCTTTAATTCCGGCGACCAGTTCTTCACCTATCTGAAAGACAGTTTCGA
CGTTCTCTATCGCGAGGGTGAGGAAGGCGCGCCCAAGATGATGTCCATCGGCCTGCATTGCCGTCTGGCGG
GGCGCCCCGGCCGCATGGCGGCGCTGGAACGTTTCCTCGATTACGTGCAGAGCCACGACAAAGTCTGGGTG
GCCAAACGTGTGGATATCGCCCGCCACTGGCATAAACACCACCAGCCGGTCGTGGCCGATGTGGTGCCTTC
CAAAGCGGACAGGCAGGCCTTTGTCGCGCGTTATGGCAGCATTTTCGAGCATTCGCCCTGGATTGCCGAGC
GCGCCTATGATGCCCGGCTCACTGTGGAAGAAGACAGTGCTTCGGGCCTTGCCGCCGCGATGACCAAGGTG
TTCCGCACCGCAGCGCCGGATGAACGGCTGGCCGTTTTGAAAGCGCATCCCGACCTTGCGGGCAAGCTGGC
GCAGGCCAGGCGACTGACGGCGGAATCAACCGCCGAACAGGCATCGGCCGGGCTGGACGCGCTGACGGACA
CCGAAAAGCAGGCCTTTACCGAATTGAACGACGCCTATACCGCCAAGTTCGGCTTTCCCTTCATCATTGCG
GTCAAGGGCCGCAACAAGCAGGAGATACTCGACGCCTTCAGGCGGCGCGTTTCCAACGACCGTGATACGGA
GTTTGAAACCGCCTGCGCGCAGGTGGAACGCATCGCGCTCCTGCGCCTGAAAGACATCCTCCCCGAAACAC
TCTAT
>BR0296
TTGTCGATCCTGCGCTTTGCTGCCGGGTTTGCCAGTGCCGTCACCATGATCCTGTGCACGGCCATCGTGCT
CAGCCATATCACGCGCGCACAAAGGGCGGGGCTTGGCGCGATGCATTTTGGCGGTGTCGGCAGTGGCATCA
CGCTGTCTGCGCTGCTGGTGGCCCTGATCCGCTTTTCGGGGCTTGGCTGGCGGGCCGACTGGATTGGCGCT
GGCCTCCTTACCCTTGTTATCATGGCTGTGGTTGCCGCTTTCGTGAAGGAGGGGCCGGTCGGCGGCAATAA
TGGCATGCGTGAGCCGCCCCTGCCCAACAGCTTCGCCTTTCATGCGCTGGCGTTTGCCTATGGCATTTTCG
GCTTTGGCTATGTCATCACGGCGACATTCCTCATAGCCATCGTGCGCGCCAATGACGGCGGGCCGGTAATG
GAGGCGGCTGTGTGGGTGGTAACGGGCCTGGCAGCAGCACCTTCCATCTGGCTGTGGGCACCTGTGGGACG
CAAATTCGGGTTATTTTCCGCTTTCGCGCTCACCGCTTTCGGCCTGCAGGCCGCGCGCATTCTTGCGCCTG
CTTCACCCAAACGCGCATTGGCGCGCATGACGGTCTGCTTCAGTATCGGGCAGATCATAGGGCCGCTCGTG
GCGGGCTTCATGGCGCAGCATTCAGGCTCGTTCACATCTGCAAGCCTTGCTGCGGCCTTGTCTTTGATCGT
TGCGGCTGCTATCGGCTTTTCAGCCGGGCGCGCAGCCCGGCTGCAT
>BR0295
ATGAAATCCTGTCTGTTGAGCCTGACGATAAGCATGTGCCTTTTCGCTCCTGCCGCCCATGCGGCGGGGGA
AGAACCGGCTGCACCGCATTTCTTCAACCAGAAAGAGCGCCTGCCCCTGCCTTCGCTGCAAGGGCTCAATC
GTCTGCGCTTCATCACCACCGTCGATTTTCCGCCATTCaACAGGCTTAACGAACGCGGGCAGCTTTCCGGC
TACAACATTGATCTGGCCAAGGCGCTTTGCCAGCAGCTCAAGATCGAGGACATCTGCCAGATCGAGGCGGT
GCCGTGGGCGGAACTGGAAGAGCGTGTGCTGGATGGACAGGCGGACGCGATCGTTGCGGGGTGGGAACCGA
CCGACAGGAACCGGGAAAAATTCACCTTCACCCGCAGCTATATGCGCCTGCCCGCACGCTTTGCGACCTCC
AATGCCAAAAGCTTTACAGAACCCGCTGCAAAGGCGACGCAGGACAAGCCCGTCGGCGTGATTGCCGGGAC
GGCGCATGAAGCGCTTTTGAAGAACTATTTCCCACAGGCAAAACCCGTGCCCTACCCCAATCGCGATGCCA
TGCTGACCGCATTGAAGGACGGCAAGATCGACACGGTTTTCGATGACGGCATGTCCCTCTCGGCCTGGCTC
GACAGCGCAGAAGGCAGCGCCTGTTGTACTTTCACGGACGGGCCCTATCTGGCGCCTCAATATCTGGGTTC
CGGCCTCAGCATCGCTGTTACCCGGCAAAATTCCGCCCTGGCAAGCGCCTTCAACAATGCCCTGCAAGCCC
TGCAACAAGAAGGTAAGCTGACCGAGCTTTACCTGAGATATTTCCCGGCAAGCTTTTAT
>BR0291
ATGCATGAGGAAAAGCTGCCCACGCTCGAAAGCGGCGAAAGCATCTTCTCTCATGTGCTGGATATCGCCAA
TGGCTATGTTTCGCTCATCCCCGCCCATTTCGCGGCCGCCCCGGATGCGGAACCCGCAGTGTCGCTTATCG
CAGCCGAA
>BR0276
ATGAATATCCAGGCCACTTCGGCACCCAAGCCGCGTACCCCGGCTGAAAGCGTGCTGATCGACAGTTTTGC
CGAGCGCATGGGCGATCTGCCCGGCAATGGCGATGTCGTGATCGCACGCGACACGGCCCTTGAAGCCCTCA
AGGTTCAGGGGCTTCCCTCGCGCCGCATCGAAAGCTGGCATTACACCGATTTCCGCACGTTGCTGAAGGGC
GTTGCGGCATTCGACACATCTGCCGGAACACAGGCTCTGCCGGCGCTGATTGCCGGTTCGTCCATCATTGC
CGCCAGCAATGGTGTCGCGCTTGCCGCCAAGGCCCCCAAAGGCGTTTCGCTGACGCCGGTTCGTGAGGCGC
TTGCCGATGGTTCGCTTGTCTCGCAGCTTAGGTCGCGTGGCTTCGACGATACGATCGGCCAGATCAACGCT
GCCTATGTCAGCGACGGCTGGATGGTTTCGATTGCCGATGACACGGAACTGGAAGAGCCGCTCGAAATACA
GAACATCCAGAATGCCGGGCAGGGGCATACGCGGTTTCCGGTTCGCATCGGCGCCAACGTCAAGGGCACGA
TCATCGAACGCCAGAATGGCGGTGAGGGGGATGCCTTCTCCACATCCGTCAACCATGTGACGGTCGGCGAT
GGTGCGCAGATCATCTGGGTGATTCTGCGCGATTTCGGCCAGGCAACACAGCTCTCGCAGTTCAATGTGAC
AATCGGCAAGGGGTCCAGGCTGACGCTTTACATCGTCAATGCGGGCGGCAAGCTGGTTCGTCAGGAAGTGC
ATGTGAATGTCGAGGGCGAGGATTCCGATTTTGAGCTTCGCGGCCTCAATCTTCTGGCCGATGACAGCCAT
ACGGACGTCACCATGACCGTGGGCCATTTGGTGGAGAATACCCGCTCCACCGAGATTGTGCGCAATGTGGT
GAAAGACCGTGCACGCGGCGTTTTCCAGGGCATGATCCGTGTCGCCCAGATCGCCCAGAAGACTGATGCCC
GCATGGCCTGCAACACACTTCTGCTTTCGGATGAAGGCGAGTTTGATGCCAAGCCTGAACTTGAAATCTTC
GCGGACGATGTGGCCTGCGGCCATGGCGCGACGGTTGCGGAACTGTCCAGGGACTATCTCTTTTATCTGAT
GGCCCGTGGCATCCCCGAAAATGAAGCGCGCGGCCTGCTGATCAAGGCGTTCATCGCCGAGATCATCGAGG
AGCTGGAAGACGAAACGCTGGTTGAAGCACTCGAAGGGGTTCTTTCCAACTGGCTCGCCGTTCATGCT
>BR0274
TTGCCGTTTCACTGGCAGGCCCGGTTTCAGCCTTGTCCTGAACTTTCTGTTCCATATCGGTCATCCGAAAA
ACTTCCTTGCCTTTTCCAAGGCTTCAGCCAATGCGTCTACTTCGGCGCGGGTATTATAAAGGGCAAAGGAT
GCACGGCATGTAGAGGTGACGCCAAAGCGTTTCAAGAGCGGTTGCGCACAATGCGTTCCCGCGCGCACCGC
GACCCCCGCCCGATCGATCACCATCGACACGTCATGGGCATGAATGCCTTCAAGCGCGAAGGAGATGATTG
CGCCCTTGTCCGGCGCATTGCCGAAAATGCGCAAGCTGTTGATCCGGCCGAGCCGTTCATGCGCATAATCA
CGCAAGTCTGCCTCGTGAGCCAGGATCGCATGACGCCCGATCTTTTCCATATATTCAAGCGCCGCACCCAG
ACCAATCGCCTGAACGATAGGCGGCGTGCCTGCTTCAAAGCGGTGTGGCGGATGATTGTAGGTGACGTTTT
CCTCCGTCACTTCCTCGATCATTTCGCCCCCGCCCTGGAACGGGCGCATCTTTTCCAGCATTTGCGCGCGT
CCATAAAGCACGCCGATGCCCGAAGGGCCATAGACTTTGTGGCCGGTAAAGACATACCAGTCACAGCCCAG
ATCCTGCACATCGACCGGCAGGTGCACTGCACCCTGACTGCCATCGACCAGAACCGGAATGCCGCGTGCAT
GGGCAAGCTCCACGATCTTCTTGATCGGGACCACCGTGCCGAGTGTATTCGACATATGCGTGATGGCAACG
AGTTTGGTACGTTCGCTCAGGCGCTTCTCGAACTCTTCGATGTGGAAAACACCGTTATCGTCGACTGGTGT
GAAAACCAGTTTCGCGCCCTGGCGTTCGCGTATGAAATGCCACGGCACGATGTTGGAATGATGCTCCATGA
TCGAGAGCAGGATTTCATCGCCCTCGCCGATGAACGGCATTCCATAGCCATAAGCGACGGTATTGATCGCC
TCTGTCGCATTCTTGGTGAAGACGATCTCGTCCACAGAACCGGCATTGAGAAAACGTCGCACGGTTTCACG
CGACTTCTCATAAGCGTCGGTGGCAGCGTTCGAGAGAAAATGCAAACCGCGATGAACATTGGCATATTCAT
TCGCGTAAGCATGAGTTACCGCGTCGATAACGCTTTGCGGCTTCTGTGCGGAAGCGCCGTTATCCAGATAA
ACGAGCGTTTTGCCGTGCACCTGCCGCGACAAAATCGGGAAATCCCTGCGTATGGCCTCGACATCATAGGC
CATGGTGAGCGGATTCTTCTGATCCATGATCGTTCCTTCTGCCTTCCCGGCAGGAAAGGAGGAAGCGGGAA
ACTCCCGCCCCGTCCTGTTCAATCAGGCCCAATTCACTCAAGCATGAACGGCGAGCCAGTTGGAAAGAACC
CCTTCGAGTGCTTCAACCAGCGTTTCGTCTTCCAGCTCCTCGATGATCTCGGCGATGAACGCCTTGATCAG
CAGGCCGCGCGCTTCATTTTCGGGGATGCCACGGGCCATCAGATAAAAGAGATAGTCCCTGGACAGTTCCG
CAACCGTCGCGCCATGGCCGCAGGCCACATCGTCCGCGAAGATTTCAAGTTCAGGCTTGGCATCAAACTCG
CCTTCATCCGAAAGCAGAAGTGTGTTGCAGGCCATGCGGGCATCAGTCTTCTGGGCGATCTGGGCGACACG
GATCATGCCCTGGAAAACGCCGCGTGCACGGTCTTTCACCACATTGCGCACAATCTCGGTGGAGCGGGTAT
TCTCCACCAAATGGCCCACGGTCATGGTGACGTCCGTATGGCTGTCATCGGCCAGAAGATTGAGGCCGCGA
AGCTCAAAATCGGAATCCTCGCCCTCGACATTCACATGCACTTCCTGACGAACCAGCTTGCCGCCCGCATT
GACGATGTAAAGCGTCAGCCTGGACCCCTTGCCGATTGTCACATTGAACTGCGAGAGCTGTGTTGCCTGGC
CGAAATCGCGCAGAATCACCCAGATGATCTGCGCACCATCGCCGACCGTCACATGGTTGACGGATGTGGAG
AAGGCATCCCCCTCACCGCCATTCTGGCGTTCGATGATCGTGCCCTTGACGTTGGCGCCGATGCGAACCGG
AAACCGCGTATGCCCCTGCCCGGCATTCTGGATGTTCTGTATTTCGAGCGGCTCTTCCAGTTCCGTGTCAT
CGGCAATCGAAACCATCCAGCCGTCGCTGACATAGGCAGCGTTGATCTGGCCGATCGTATCGTCGAAGCCA
CGCGACCTAAGCTGCGAGACAAGCGAACCATCGGCAAGCGCCTCACGAACCGGCGTCAGCGAAACGCCTTT
GGGGGCCTTGGCGGCAAGCGCGACACCATTGCTGGCGGCAATGATGGACGAACCGGCAATCAGCGCCGGCA
GAGCCTGTGTTCCGGCAGATGTGTCGAATGCCGCAACGCCCTTCAGCAACGTGCGGAAATCGGTGTAATGC
CAGCTTTCGATGCGGCGCGAGGGAAGCCCCTGAACCTTGAGGGCTTCAAGGGCCGTGTCGCGTGCGATCAC
GACATCGCCATTGCCGGGCAGATCGCCCATGCGCTCGGCAAAACTGTCGATCAGCACGCTTTCAGCCGGGG
TACGCGGCTTGGGTGCCGAAGTGGCCTGGATATTCATGATCCAACCCTCCTCAGGCGGCTTCGCCGATTAC
GTCGGCATAACCATTTTCTTCCAGATGCAGCGCGAGGCTCTTGTCGCCGGACT
>BR0270
ATGACTGAAACAGGTTTCAGTTTCTCAAGCGGCTTCATAGGCCGTTCTTGGTTGAAAATTCAAGAAAATTG
CGACTTCAGATATCTCGAAGGCGTTGTTAAGGAGATAGGTAAGGTGGTCGGGAGCGGCAAGAGGCTCTATC
TGGATTATAATGCCAGTGCGCCTTTGCTTGATGAAGCGCGCGATGCGATCATCGCGGCATTGGGCATTACG
GGCAATCCATCCTCCGTTCACCGCGAGGGGCGGGCTGCCCGTGCGCTGGTCGAATCCGCCCGCCGCAGTGT
TGCGACCCTCGTTAATGCCAGGCCGGAGCATGTCTTTTTTACGTCCGGTGCGACAGAGGCGGCCTCGACGC
TGCTGACCCCCAATTACATGATgGGGCGTTCGCCGGTGCGTCTCTCACACCTTTATGTCAGCGCCACCGAA
CACCCATGCATGATCGCCGGCGGACAGTTTTCGCCCGATGACATCA
CCGTTCTTCCGGTTGATGCCAATGGCATCCTGCGCCTGGACATTTTGCGCGGCCTGCTTGCCGCCCATGACA
GGAGCAAGGGTCTTCCGCTGGTTGCAGTACAGGCAGCCAATAATGAAACGGGCGTGATCCAGCCGGTGGCGG
AGATCGCAGCCATCGTAAAGGCTGCGGGCGGCATTTACATCGTCGATGCCGTGCAGGCTGCAGGGAGAATTA
AATTAGATATTACAGATAATTGCG
GTGATTATCTCATAATTTCCTCACACAAGATCGGTGGCCCGAAGGGCGTCGGCGCGGTCATTGCGATCTCC
GACCTCATGATGCCGCGGGCGCTGGTGCGCGGCGGCGGGCAGGAAAAAGGCCACCGGGCGGGAACAGAGGC
GCTGCCCCTGATTGCCGGTTTTGGTGCGGCTGCCGATGTGGCAAAAGCGCGAATCGACGGCGAAGGCTGGT
CATCCCTCATGCGGGATCGTCTGGAGCGTGGCCTTGCCGAAATTGCgCCAGCGgCGATCGTTCATGGCAGT
GCTGTGCCGCGCTTGCCGAaTACGACTTTCTTTTCCCTGCAAGGCCAGAAGGCTGAAACGGTGCAGATCGC
TTTTGATCTTGCAGGTATTGCGCTTTCGGCCGGGTCGGCCTGtTCTTcGGGCAAGGTGGGGCCGAGCCATG
TTCTTGCTGCCATGGGGCAGGAAGACGGGCCGGGAGCCATCCGTGTTTCGCTTGCAACGGATGCGGGCGAG
GAAACAGTGGATCGATTTCTTGAAGCCTTGCGCAAAATCGTCGAGCGTCACCAGCGCACGAAGCACGAATT
GGGCGCGGTT
>BR0202
ATGCACAAGCCGAAACTGCTTCTGCTGGACGAGCCAACCGCGGGTGTGGACCCCAAGGCGCGGCGTGATTT
CTGGGATGAAATTCATCGGCTGGCAGATGGCGGCCTCACCGTTCTGGTTTCCACCCATTATATGGATGAGG
CGGAGCGGTGCCATCGCATCAACTATATTTCCTATGGCAAGCTGCTTGCAACGGGCACGGTCAGTGAAGTG
ATCGAACGCACGGGCCTCACCACTTTCTTCGTCAGTGGAAAGCGGCTCGGCAATGTGGCGCGAGAGCTTGA
AGAAATGCCGGGCGTCGATCAGGTCGCGCCTTTCGGGGCTACATTGCATGTGGTCGGCTCTGACAGGGAGA
AGCTGACGGCCGCACTCGACAGGATCAAGCATCGCGAAGACATCCATGTAGAGGCAGGCCAGACCAGCCTT
GAGGATGTTTTCATCCAGTTCATGGCCGGGGCGAAGGACAATATGC
AA
>BR0154
TTGGTCAGAATGCGGGGGCAGGCAATTGCGATAAACTGGCAGCGGTATATCTCATGCCCCCGATATACCGG
AACCAGGCTTCGAGGGAGCTTCTCCCCCGAAGTCCGGTTCCCGGAACCGTCACGGGGCAAGCCCGGTGTCC
AGCTAAAGAGCCTTGCCATGAAATGTGCCTACTTCGTGCGCCCCCATATTGGCGGAACCTATTCGGTTTTC
ACACGGTTGCGTGCTTCGCTTGCCCGGTCTGGCATGGATCTGCGCTGGCTTGCAGCCGGTGCGCCGGGCGA
AGCGGTGGCCGTGCCGCGCGAGCAGGGGCTGGAAGATGCCATGCGCCGGGGCGACATCATAGATCGCATGG
GCGTTCTGGATGAAGAAACCCGCGCCAAACGCTTTGTCGATTTTCTTCGCGAAAACAGGTTTGAAGCAGTT
TTTGTGAATGTGCTTTCTCACCGTTTCGAGACCAATCTCGTGCGCTATCTTCCCGCGGATATTCTGCGTGT
GATGATCGTTCACAGCATCACGCCCGGCACCTATGCGGCGGCGCAGGCGATCCGCGATCATGTTCACGCGA
CGGTCGGTGTTTCCAAACGGTGCCGCGACGATCTGGTTCGTGGCTATGGCTTCGACCCCAGCCGCACACTC
GTCATTCCCAATGGGCTGACGCCCGATCCGCATGTCCGGTGCGCAGCGCCGGAGATTGAAGGCGGACGGCT
GCGACTGCTCTATCTGGGGCGCATCGATGACAGTTCCAAGGGGGTCTTCTGGCTGCCCGCGATCCTGCGGC
GGCTGCAATGCGATTATCATCTGACGGTGGCGGGCGACGGGCCGGACCTCGCAGCCTTGCGCATCAAGCTG
GCTCCCTTTGGCAACAGGATCAGTTTTACCGGCTGGGTCGCGCCATCCGATGTTTCATGGCTGGTGAGCCA
GCATGACGTGATGGTCATGCCATCGCGCTATGAAGGTTTCGGCTCGACGCTGATTGAGGCAATGAGCCAGG
GATGCCCGGTGGTGGCGTCGAGGATACCGGGCGTGACGGATACTATCGTCACCGATGGTGAAGACGGTTTT
CTGTTTCCGATCGGCAATTGCAGGCAGGCGGCGCAGCAGATCGAGAGGCTTGCCGCCGATCCGCGCCTGCG
CGTGGCGATGGGTGCGGCAGGCGTGCGCAAGGTGATCGCGGAATTCGACAGCGAAATCGTCGGGCGCGCCT
ATGCGGGATTGCTCCAGCATCTGCGTGAAAATCGCCCGCCCATTACGCCGCCTCTGGCTCTTTCGCAGTGG
TCCATGCCCAATGCCCTGCACAAAAACCTGCGCAGCCGCTTGCCGCAGCCGGTCAAGAACTGGCTGCGCCG
CGTGCGGGAGCGCGTGCACACGGTATGGTCGACGGCC
>BR0149
ATGTATATGGAACGCGAGACGGGCAAGATGAACGGGGACACGATGGTCCGCACTTTGCGGATAGGATTTTC
GGTTCTGGCGCTTGCCGCCTTTGCTGCGCCGGGCGCTTCCTGGGCGCAGCAGGCCGGTGATGGGTCCGTGC
CCGGCCTCAAGCTTTCCAACAGCAAGGATCCCGTCAAGATCGATGCCGACAAGCTGGAAATGCGCGACAAG
GAAGGCGTGGCCGTTTTCACCGGCAATGTGGCCGTTTCGCAGGGCGACGCCTTGCTGAAGGCTGGCCAGAT
GACCGTTTATTACAGCAAGGCCAAGAAGGGCGCTGAAGCGCCCGAACCTGCAAATGCAGGCGTGGGCGGGA
TTGGTTCGTCGAGCATTGACCACATCGATATTGCCGGCAAGGTTTATCTCAAGTCCGGCACGCAGGTCGCG
ACCGGCGACACAGGCCGCTATGACGCGAAGAACCAGATCATGGTTCTCCAGGGCCAGAAGGTCGTGCTGAC
GGACGGTGACAATATCGTGACCGGCTGCAAGCTGACCGCCCATCTCGATACGGGGCGGGCCAATGTAGAAA
GCTGCAAGGGCCAGACCAAGGGGCGCGTGTCGATCATCATGGCTCCGAAGGATCAACAGCAGGGCGGTGCT
GCGCCCAAGCGGAAC
>BR0143
ATGGATCTTAGCGATCTGATTCAGCCCGGGGCGATTATCCCGGCGCTGAAAGCCAGCTCCAAAAAGCAGCT
TTTGCAGATTCTGTCTGAGAAGGCTGCGGTACTGACCGGTCTCCCTGAGCGTGAAGTTTTTGAAACAGTTC
TCCAGCGTGAGCGCCTCGGTTCCACCGGGGTGGGGAATGGCGTCGCCATTCCGCATGGCAAGCTGGCGAGC
ATCAACAAAATCGCCGGAATCTTTGCGCGCCTTGAAACGCCGGTCGATTTTGAGGCGCTGGACGATCAGCC
TGTCGATCTCGTTTTTCTGCTGTTGGCCCCGGAAAATGCCGGTGCCGATCATCTGAAAGCACTGTCGCGCA
TCGCCCGTTTGCTGCGCGACCCCGATATGGTGGCCAAATTGCGCGGCACCCATGACGCGCAGGCCCTCTAT
TCATTCCTGACGGCTCACCCGACGTCCAACGCCGCC
>BR0139
ATGTCTCCCAAAAAGCTGGAAATGCTCGTGAGGTCAGTAACCATGGCTTTACCATTGCGATGCGGGAATTC
GGCTTGCACCCTTGAAGTCCGGCTTTTCACAAGCCATATCAGTCTT
>BR0119
ATGACGATGCAATGGTTCTCGCTCGCGCGCCTTCGCGCCATGCTGATGAAGGAATTCATCCAGATGTGGCG
CGATCATATCACTTTCGCGATGATGCTGATCGTGCCGCTCATGCAATTGCTCCTGTTCGGTTTTGCGATCA
ACAATGATCCCAAAAGCCTGCCAAGCGCCCTCGTTGCACTGAGCAATGATCATTATACGCGCGCAATGATT
TCGGCACTGGAAACGACGAACTATTACCGCTTCGACCATGTGGTGCATAGTGCTGCCGAAGCCGATGCGCT
TCTGGCCAATGGCAGCGTTTCCTTTGTGGTGACGATCCCGTCCGATTTTGCGCGGCGCGTCGATGCGGGCG
ACAGGCCGCAGCTTTTGATCGAGGCTGACGCCACCGATCCATCCGTGGCAAGCGGCGCAATCTCTACGCTC
AGCACGGTGGCAAGCCAGGCATTGCTGCGCGAGCAGGGCAGACAGG
CCGAAGCGGAAAACGCCTCGCGCAGCCAGCTGGAGGTGGTGGTGCACCGCCGTTACAACCCGGAAGGCATA
TCGCAATATAATATCGTGCCGGGCCTGCTCGGCGTGATCCTGCAAATGACGATGGTGATGATGACGGCCAT
GGCGCTGACGCGCGAAACCGAGCGTGGCACGATGGAAAATCTGTTGGCCATGCCCGCCACGCCTGCGGAAA
TCATGCTGGGAAAGATATTGCCCTATCTTGCCGTGGGCGGGGTACAGATGGTTGTGGTTCTGGTTGCCGCC
AAACTTATTTTCGGCGTGCCTTTTGTCGGGTCGCTGACGCTTCTCCTGAGCAGTGTGCTGATTTTCGTGCT
GTCGCTCGTTCTGCTTGGCTATACGATTTCCACCGTGTCGCGCACACAGATGCAGGCAATGCAGCTTACCT
TCTTTTTCTTTCTGCCCTCGCTGATGCTTTCCGGCTTCATGTTCCCGTTTCGTGGAATGCCGGACTGGGCC
CAGGCGCTGGGCAATATTTTTCCGCTCACCTATTTCCTGCGCATCGTCCGTGCGGTGATGCTGAAGGGGGC
AGGGCTTGCCGATATTGCGGGAGAGGTGATGGCGCTTATCCTGTTCGTTTTTCTCTTTGCGGGCTTGGCAC
TGTTGCGCTTCCAGCGCACACTGGAT
>BR0118
TTGCTTCCAGCGCCAGCAGGCGTCTGCTCCGTGCTGGAAGTGTTTGAAAGGATGATGCCCGTGTCTTTATC
GCAAACGCGACTTCTGGCTGATTTTCCTGCTTTCTTCCGGGTTTCGGGCGAAGTGGTGGCGGTCGTGGGTG
GTGGCGAGrAAGCCTTGAACAAGGCTCGCCTCGTGGCGCAGACGGCTGCGCGCCTGCGCATCATTGCTGAA
AAGGCTGAGCCGCATCTTGCCGCTTTTATTGAAAGCCATGGGGTGGAGCATATTGCGTCACCCTTTGCGCC
GGAACTGCTGGATGGTGCGAAGCTCGTTTTTGTCGCGACCGGCGATGAGGCCGGGGATCGTGCCGCCGCTG
CCGCCGCAAGGGCCCGCAAAATTCCGGTCAATGTGGTGGACCGTCCTGCCCTTTGCGATTTTCTGACACCT
GCCATCGTCAATCGTGCGCCGGTCACGATCGCCATCGGTTCGCAGGGAACCGCCCCGGTTTTGGCGCAGAT
GGTGCGTGCGCGTATCGATGCAGCCTTTTCGCCGCGTCTTGGCGAGCTTGCGCATTTTGCGGAAAGCTGGC
GCCCTCTGGTCGAGCGGGCGCTTCCAAAAGGGCTTGCGCGGCGTGGTTTCTGGCGCACATTCTTTTCGGGA
AAAGTGGCCCGCGCGGTGGAGAGTGGCGACCACGAGGCGGCGCACAGGGCTGCGGCGGAACTGATCGGGCA
GGGGCAGGACGCTTCCGGCCATGTCTGGCTGGTTGGGGCCGGTCCCGGTGCTGAAGACCTTTTGACGCTGC
GCGCCCAGCGCGTGCTGATGGAAGCCGATGTGATCGTTCATGATGCGCTGGTGCCGGAAGGCGTCATCGCC
ATGGGACGCCGCGACGCCGAGCGCCTGCCGGTCGGCAAGCGCAAGGGTTGCCATTCCAAAAGCCAGGATGA
GATCAACCGCCTGCTGGTGAAGCTGGGGCAGGAGGGCAAGCGCGTGGTGCGCCTGAAATCCGGCGATCCGC
TGGTCTTTGGCCGTGCGGGTGAGGAAATGGCCGCCTTGCGCGCCGCCGGAATAGGCTTTGAGGTGGTGCCG
GGCATCACCTCTGCCTTTGCCGCTGCCGCCGATATGGAACTGCCGCTGACCTTGCGCGGCGTCGCGTCCTC
GCTCGTCTTCACCACGGGCCATGACATGACCGGCGATGTGCTGCCCGGCTGGGCGAAGCTTGCCGTCTCCG
GCGCTACGATTGCCGTCTATATGGGCTCCACGGTTGCCGCTTCCGTTGCCTCGCGTCTGATTGCCGCCGGC
CTGCATGAGGATACGGCGGTTGCAGTGGTGGAAAATGCAAGCCGCAGCAACAAGCGCATGTTCCATGGCAC
GCTGAAGGATTTGCCGCAGCTTGAAAACCGCAAGGAGCTTTCCGGCCCGGTCATGGTGGTCATTGGCGATG
CCGTCGCCGGTGCCGCCATCGGCAGGGCGCAGGCCCTTTCCCTGAAACCTGTTCCCGTCGCAGCC
>BR0117
ATGTATCGTTATGATGAATTCGACCGGGATTTTGTTGCAGCCCGCGTTGCCCAGTTCAAGGATCAGGTTGC
GCGCCGCCTTTCGGGCGAGCTGACGGAAGACCAGTTCAAGCCCTTGCGGCTGATGAACGGCCTTTACCTGC
AATTGCATGCCTATATGCTGCGCGTGGCAATCCCCTATGGAACGCTGTCCAGCCGCCAGTTGCGCAAGCTT
GGGTCTATCGCGCGCACTTATGACCGTGGCTTTGGCCATTTCACGACGCGCCAGAACATCCAGTATAACTG
GCCTGCGCTGAAGGATGTTCCGCAGATTTTGGCAGAGCTGGCAGAGGTGGAAATGCATTCCATCCAGACCT
CCGGCAATTGCATTCGCAATGTGACTGCCGACCATTTTGCAGGTGCTGCCGCCGACGAAGTGACGGACCCA
CGCCCGCTTGCCGAAATCCTGCGGCAATGGTCGTCGCTGCACCCGGAATTTTCCTATCTGCCGCGCAAGTT
CAAGATTGCGATTGTCGGGTCCGAGCATGACCGTGCGGCCATTCAGGTGCACGATATCGGCCTGCAACTGA
AGAAGAACGAGGCGGGCGAGCTTGGCCTTGCCGTTTATGTCGGCGGCGGGCAGGGCCGCACGCCGATGGTG
GCCAAGAAAATTCGTGACTTCCTGCCCATCGAGGACATGCTGACCTATGTGACGGCAATCGTCCGTGTCTA
TAATCTTCATGGTCGGCGTGACAACAAGTTCAAGGCGCGCATCAAGATCCTTGTGCATGAAACCGGCATCG
AACAATTGACCCGCGAGATCGATGCGGAATGGGAAGAAATCCGCCATGGCGAATTGAAGCTGCCGCAGCGC
GATATCGATGTCATTGAAAGCTATTTCCGTATGCCCGACCTGCCGCAGCGCCCCGAAGGCTGGGAAATTCT
CGCCGTGGCACAGAAGGCTGATGCGGAATTTGCAAGCTGGGTCGCGCGCAATGTCACCGCGCACAAGCATC
CCGATTATGCCGTCGTCACCATCTCGCTGAAGCCTGTCGGCGGCATTCCGGGCGATGCCAGTGCTGAACAG
ATGGAACTGGTGGCCGATGTGGCGCAAACCTGTTCCTTCGATGAAATCCGGGTGAGCCACGAGCAGAACCT
CGTGCTGCCACATGTGGCCAAGGCCGATCTGCCGGCAGTCTACGACCGCCTGCAATCGGGCGGACTGGTGA
CACCCAATGCCGGTCTCATCACCGATATCATTGCTTGCCCCGGTCTTGATTATTGCGCGCTGGCCAATGCA
CGCTCCATTCCCGTTGCGCAGCGCATTTCCGAGCGCTTCGCCGATCTGGACCGTCAGCTTGAAATCGGCGA
TCTCAAGATCAAGATTTCCGGCTGCATCAATGCCTGCGGCCATCATCATGTCGGCCATATCGGCATTCTGG
GCGTGGAGAAGAAGGGTGAGGAGCTTTACCAGATCACGCTTGGCGGCTCGGCGGACGAAAACTCGGCCATT
GGTGAAATCACCGGGCGCGGTTTCTCGTCGGAAGAGGTGGTCGATGCCATCGAAACCATTGTCGATACCTA
TCTGGGCCTGCGCGAAAGTGCGGAGGAAACTTTCCTTGCCGCTTATCGCCGGGTGGGAATGGAGCCTTTCA
AACGCGCTCTTTATGGGGAGATAAGAAGTGCTGCC
>BR0105
GTGGGGGCCTTCCGCCAGGATGAGCTGTTCCAGCTCGTTTGCCAGGCGCGTTGCGAACTGCTCTTCGCTCT
CACCCGGCAGCGCATCGTGGTAATGATGCGGGCAGCCGGTATGAAGAATACGATCGATGGGCAGATCGAAG
GAGCGGTGATTGTTGGGCAGGCCCGTCAGGCTGGCGGAGGCAATGGTCACGCCATGATAGCCGCGCTTGCG
CGAAATGATCTTCTTGCGCTCAGGCTCGCCCAGCGCATTGGAGCGATACCAGATGAGCTTGACGACGGTAT
CATTCGCTTCCGAGCCGGAATTGGTGAAATAGGCCTTGTTCATCGGCACCGGCGCCATGGCGACAAGCTTT
TCAGCCAGATCGATCACCGGGCCATGCGAGCGATAGGAAAAGGTATGATAAAATGGCAGTTTCTTCATCTG
CCGCGCAGCAGCTTCCGCCAGACGCTGTTCCGAAAAACCAACGCCGACACTCCACAGGCCCGACATGGCTT
CAATATAGCGTTTGCCGGCAATATCTTCGACATAGATGCCGTCGCCGCGCTCAATAACGAGCGGGCCTTCC
GCTTCAAGACGGACAGCATCGGTGTAGGAATGCAGGTGATAACGGATATCGCGGGCTTCAAGCGAGTTCGG
CTGTGCAGTCATTCATTCATCCTCCAAGAACGA
>BR0067
GTGAAGTGGTTCAACACCACCAAGGGCTTCGGCTTCATTCAGCCTGATCAGGGCGGCACGGACGTTTTTGT
CCACATCTCCGCCGTTCAGCGCGCTGGCCTCACCACGCTTGATGAAGGTCAGAAGGTCTCTTACGAGATCG
TTCAGGACCGCCGTTCGGGCCGTTCGTCTGCCGACAATCTCGTCGCAGCA
>BR0057
GTGTGTCGAGGGCGGATTGGCCTTCACAAAGCGCCTCGCGCTATCCGAGCATCGCGCGAGGTGAGGCGGGC
AAGGCAAaCTGCCGGTGCGGCCAGATTCCTGAAAAGGGCCGGCGCCGGGCGAAGGAAAGGACTGGGTATGG
AAAAGTTCCCTATGACCCCTCGCGGTTTTGAAAAGCTCAAGGAAGAGCTGCGCTGGCGTCAGCAATCAGAG
CGCCCTCGGATCATTGAGGCAATCGCCGAAGCGCGCGCCCATGGTGATCTGTCGGAAAACGCCGAATATCA
CGCCGCCAAGGAAGCCCAGAGCCTCAATGAAGGGCGCATCAACGAATTGGAAGACCTGGTTGCACGCGCCG
AGGTGATCGATGTTTCCAAGTTGACGGGCGACAGGATCAAATTCGGTGCGACCGTTACGATGATCGATGAA
GACACCGAAGAAGAAAAAATCTACCAGATCGTCGGCGATCAGGAAGCCGATGTGAAGGAAGGCCGTATTTC
GATTTCCTCTCCGATCGCGCGCGCCCTTATCGGCAAGGGCGAAGGCGACACAATTGAGGTCAACGCACCGG
GCGGCTCGCGTTCCTACGAAATCATCGCTTTGAAATTCGTC
>BR0050
ATGCCGAAGGATGCCAATTCAGCCTATGATATTTTCGGCGGCTGGGTGATGGCACAGATGGATATGGCTGC
TGGAATCCGTTCTGCCGAGCGCGCCAGGGGGCGCACGGTGACAGCAGCGGTGCGCGAAATGGCATTCGCCC
GACCAGTGAAGATTGGTGACACGCTCTGTGTTTATACGGATATAACGCGGGTGGGCCGTACGTCCATCACG
CTCCGGGTTGAAGCGTGGGCAAAGCGCTATCTCAGCGATAAGCGTGACCTCGTCACGCATGCTGAATTCAT
CATGGTTGCGCTGGATGCGGACGGCAAGCCTACCCCGGCACCGCAGGAAGAT
>BR0049
ATGAAACTGTCGGCAAGAGCTTCAATATTTACATTAGCACTTCTTGTTTCAACAAATGCTTATGCATCATC
GGCCATCGTCACATCGACGGTTAATCTGCGCACCGGCCCCGGCACCCAATATGGAACAATCGGTGCGATAC
CGAACGGCGTCGGCATTATGGTGGCGGGCTGCACCCGCGGCTATGGCTGGTGTCAGGTCTCCTATGGCGGC
ATGACCGGCTGGGCGGCATCGCGCTATATCGCCATTCAAACCGGCAACGGCTACACGACGAACGACAATTT
CGGATCGACCGCCGCAGCGGTCGGCATACCGCTCATTGCTGGAATCGTCATCGGCTCGGCCATCGCAAACA
GCGGCCCTGCCTGGAGGCCCGGCCCCTATCGCGGCTGGGGTGGCAGGCCCAACATCTATAATGGCTGCATC
GGGCGCAATTGCCAGTCGAACTGGGGACCGCCGCGCCCGCATTGGACGGACCGCCCCGGCTGGCGCGACAA
TCCGGGTTATCATCCCAATTTCGGTCATGGTTTCGGTCACGGCTTTGGTCATGGGCCCCGCATGCGTCTCG
GCGGCAGGCGG
>BR0048
GTGAAGGGGATTTCGCTTCCACACTCTTTTCATTTGAACGCTGAAATTCCATATTGGGAGCATGGCTTCCT
CAAAAGACAAACATATCCGGGATTCGCGCAACAAGGC
>BR0042
ATGAGCGAACCGAACCCAAAACGCGGATTTCGTCCGCTGGCGGATATGGCGTCGGGTCTGGTTGACCCGGT
GCTGCGCAAGCGCGCGGGCATCAATCTTGCTTTGCTGCAAGCCTGGGAGGATATTGTCGGCCCGGCCATCG
GCGCGTCCTCGCGACCGCTTCGCATTCTCTGGCCGCGTCGTATTCGCGAAGACGATCCCTTCACGCCTGCT
ACACTGGTTATTGCCTGCGAGGGTTTCGCGGCACTGCAAATCCAGCACGAAACCGGTGAAATCATCAGCCG
GGTCAATGGTTTCCTCGGCTTTGCGGCCATTGGCCGCATCCGTATTAAGCAAAAACCGCCCGTCATTGCAG
TGAAGCGCCGTGTGAAGCGCCTTGCGTCGCTCGGCCCCGCCGAAGAGCGCAGTGTGGACAAGGCAACGGCG
GGCATCGAGGATGATGCGCTGCGCCAGGCACTTGCCCGCCTCGGCAGGAATATCCTCGCTGAAAAGCGCAT
GCTGGGCAAAAAGGGCGGCTCGAAAAAC
>BR2175
ATGGCAAATTATTCCGTTACTTCCGGACTGTTGGCAGTTACCACCCGCCACGGCACCAAGCGAAGAGAGAG
AAAGTACATGAAAAAGCTCATGAGCGAAGCCGACGCACAAGGGCTTGCCATTGAAGCACTGGCCTGGCTCG
CGCAGGACAAGGACCTGCTGCCGCGCTTTCTGGCGCTGACCGGCATCGAGGCTTCCTCTATCCGGCAGGCG
GCACGGGAGCCCGGTTTTCTGGCTGGCGTGCTACAATTTTTCCTGGGACATGAACCGACGCTTATCCGCTT
CTGCGAGGAGACGGGCCATGACCCCGCCCTGTTTGAAAAAGCGGTTGCGCTTTTGCCTGGCGGCTTCAACC
AGCATCTT
>BR2148
ATGCTACTGCCACCCATCAAAGAAATGCGGGCCCGCCATGGCGCGCACGCGGTTTGCCGCATCTGTGCCGA
GCGCGGCTGCATCCGAGACAACCCCTTCGGCCGTCACTTCATGGGCCTGCCGGCCATCCGGCGTCAGGATC
ATCCCCCGGAAGGACAACCGGTCGCCCTCGACCGTTGCAAGACCGGCAATAGGCGTACGGCAGGAACCGTC
GAGAGCGCCGAGAAAGGCCCGCTCGCAGGCAAGCGCAATTTGCGTTTCGCGATGGGCGAGCGGCGCAAGAA
GCACATCGATCCGCTCGTCGCCAATGCGGCTTTCAATGCCGATTGCGCCCTGCCCCGGCGCAGGCGGGAAA
ACGGATGGATCGAGAAGATCGGTGATGACATCGGCAAGGCCGAGCCGGCGCAAACCGGCACAGGCAAGGAA
AGTGCCATCCACTTCGCCCGCATCGAGCTTGCGCAGGCGGGTATCC
ACATTGCCGCGATACATCACCACCTCGATATCCGGGCGAAGACGGCGAATAAGCGCCTGACGGCGCAGCGA
GGACGAGCCAACGGTGGCGCCCTGCGGCAAATCCATGAAACGCCTTGCGCTGCGCCCGATGAAAGCGTCGC
GCGGGTCCTCACGCTCCAGAAAAACGGAAAGGTGAAGCCCCTCTGGCAAGACGGTGGGCATATCCTTGGTC
GAGTGGACGGCAATATCGATTCGCCCATCCTTCAGCGCCTGCTCGATTTCCTCCGTAAACAGCCCCTTGCC
GCCAACTTCCGAAAGCGGGCGGTCCTGAATGCGGTCGCCTGCGGTGGACATGGGCAGGATTTCAATCGCAT
CTTCCGGCAAACCATGAGCCTCCTGCAAAAGGCGGCGCGTGAGGTAAGCCTGTGCCAGAGCCAGCTTGCTG
CCCCTGGTGCCGATCTTCAACGTGCCATTCTTGAAGGATGCTGTTTGCATATTGCCTCGTCCATGGTACCG
GGTTTCATCATTCGTTTTCATGCATGTCCAAAGGCAAAACCGGTTTCCACTTTTGCTGAACATGCGCCAAT
A
>BR2146
ATGCAGACTGTGCTCAGCATTGCTGTCGATGCCCTTGCCTATGGCATGGTGCTTTTCGTCATCTCCATCGG
CCTTTCCGTCACCATGGGCCTGATGCGCGTCGTCAATCTGGCACATGGCGCCTTTGCGATGATCGGTGGCT
ATTTCGCATCCTATGCCGCACAACAGCTTGGCCTCAATTATGGCTTTGCGATCATCATTGCGGTCGTCGGC
ACGATGATTATCGCCGTGCCGGTGGAAAGGCTGCTTTACCGCCGTATCTATGGTGCACCGGAATTGACACA
GGTATTGATGACCATTGGCGTCACGTTCTGCATCATCGGCATCACCAATTATATTTTCGGCCCGACGCTGA
AGACCATTCCCCTGCCGGAAATGCTGCGCGGCTCGGTCGATCTCGGCTTTCACGCTATTGCAAAGCACCGC
ATTTTCGCTATCGGCTGCGGCCTCGCGGTGGCAGTCGGCCTATGGTACGCCGTCGAGCGCACCGCTTTCGG
CGTGAAACTGCGCGCTGCCGTCGATAATGCCAATATGGCGGCAGCCCTCGGCGTGCGTACCGAGATCGTCT
ATGCGGTGAGCTTTGCGCTCGCCATCGGGCTTGCGGCCTTCGGCGGCGTGGTCGGCGCGGAGCTTTTGCCC
GTCGAACCCTATTATGCGCTGCGCTACATGGTGACTTTCCTTGTCGTGGTTTCGGTCGGCGGGGCCGGTTC
TATCCCCGGCGCACTCGTCGCCTGCCTGTTGCTCGGCGCAATCGACACGACCGGGCGTTACCTCGCACCCG
AATATGGCGAATTCTTCTTTTATCTGGCGGTGATCGTGATCGTTACCCTGTTCCCGCGCGGCCTTGCAGGA
AGGCTGGCCCGCAAA
>BR2145
ATGCGCGTTCTTGGAATAGAAACAAGTtGCGACGAAACAGCCGCCGCAATCGTCGAGCGTGACGATATGGG
CGAAGGGCGGATTTTGTCCAaTGTGGTTCTGAGCCAGATTGCCGAGCACGAGCCCTATGGCGGCGTGGTGC
CGGAGATCGCCGCCCGCGCCCATGTCGAGGCATTGGACAGGCTGGTGGACCGTGCCCTGAACGACGCGGGC
CTGAAACTGTACGAGGTGGATGCAGTGGCCGCTACGGCTGGCCCCGGCCTCATCGGCGGGCTGATCGTCGG
GCTGATGACGGCAAAGGCGCTCGCCATGGCTGCACAAAAGCCCTTCTATGCCGTCAACCATCTGGAAGGCC
ACGCGCTGACGGCGCGGCTGACCGACGGCCTGCCATTTCCCTATCTGCTGCTTCTCGTTTCCGGTGGCCAC
ACGCAGATGGTGCTTGTGCGCGGCATCGGCGATTATGAGCGCCTCGGCACCACCATCGACGATGCGCTGGG
CGAAGCCTTCGACAAGACCGCAAAGCTTCTCGGCCTGCCTTATCCCGGTGGCCCGGCTGTCGAGCGCATGG
CGCTACAGGGCGACCAGAAGCGTTTTGCCCTGCCGCGCCCGCTGAAAGGCGAAGCACGGCTGGATTTTTCC
TTCTCCGGCCTTAAAACCGCCGTCCGCCAGACCGCAACCGAATTGGTGCCTCTCACCGATCAGGACGTCAC
CGATATATGCGCCTCGTTTCAGGCTGCTGTCGCCGATACGCTTTCCGACCGTGTGGGCCGCTCGCTGGAGC
GTTTCAAGACGGAATTCCCGGATTGCGCCACGCCCTCCCTGGTGGTTGCAGGCGGGGTTGCAGCCAACAAG
ACCCTGCGTGCCGCACTGGAAAACCTCTGCACCCGGCATGGCTTTGCCTTTATCGCTCCGCCGCTTAATCT
TTGCACCGACAATGCAGCGATGATCGCATGGGCGGGGGCTGAGCGCGCCGCGACCCAAGCGCCTGATTCGC
TTGACATCGCCCCGCGCTCGCGCTGGCCGCTCGACGAAAAATCCGCGCCGGTCTTCGGCACGGGACGGCGG
GGAGCAAAGGCA
>BR2135
ATGAACTTCAAGACCCGTATGGCTATGCTTGCCGTTGCGGCAACCGGCCTTCTCGCCGCCACCGCAATCGA
GGCTTCAGCAGAGCAATGCGGCGGTGCCTCCTGGTACGCCCTTACTTCCCGCACGGCTTCAGGCGAACGCA
TGAACCCGGCCGGACTGACCGCAGCCCACCGCACCCTGCCGCTTGGCAGCAAGGTCAAGGTCACCAATCAG
AAAAACGGCAAATCGCTCGTCGTGCGCATCAATGATCGCGGTCCGTTCATCAAGGGGCGCGTACTCGATCT
TTCCAAGGGCGCTGCAAGCCGTCTGGGCTTCATCGGCGCCGGGCACACCAAAGTCTGCTTCAAGCCACTT
>BR2134
GTGCGCCCCGAGCATGTCCGCTCACGGGCCAGCAAAAATATTCGGTTCGGGACAGGCTTTATGCGGCAAGG
ATATTCGCCCTCATACCCGCTGCGTGACGAGCGGATTACGGAAGACAGGTTGCGGTTTGCCAATCTCGTCC
GCAGGCAGGTCTATATCCTTCTGGGGCTGGGGCTTCTTTCGCTCACCGCCATGGCGGTTGGCGCGCTTGCC
ACATGGAATGTCGCCGATCCAAGCTTCAGCCATGCCACCGACAATCCTGTCACCAATGCGCTCGGCTATCC
GGGCGCCGTGTTCTCCGATCTGGCCATGCAGTTCTTCGGCCTTGCCAGCGTTCCGGCGCTGCTGCCGCTTG
CGGTCTGGTCGCTTCTCCTGATGATCCGGGGTTATATCGGGCGTATCGCCCGGCGCAGCCTTGCGTGGGTG
GGGGCGGCGCTGCTCTTTGCCGCCATTGCAAGCTGCTTAGCCGTGCCGCAAAGCTGGCCAATGCCAATCGG
CCTTGGCGGTGTTTTCGGCGATATGCTGCTTCGGATACCGGGTTTCTTCCTTGGCGGGTTCCCGCAAGGGG
CTATTGCCTCCGCTATTGCGCTGGTTCTGGCTTTTCCAGCCTTGTGGTTCTGTTTTTTTGCAAGCGGCATC
ATTGGTCGCGGGGCGGAAGCGGTCAATCCGGCCATGCTTTCGGCAAATCGTTCTGCCGATGATGAATTCGC
GGATGAGGATGCAGACAATGAAGCCGGTGGCGGTTTCCACTTCATCGGTGCGCTGACCCATCTGGTGCTGA
TGACGACAGCCACGATCAGGCGCATGACCGGACTTGGCCGCCGCCGCTCGCGCGAGGATGATTTCGACGAC
ATGCGCATGGTGCGCCGCAGTGCCGAAACCCGCAATGCGCCGCGCCGCGAACCCGGTTTCGGGGCGCCTGC
CGCCGATGACGAACCGCCTTTCGATATGGACGATATGGATGATGGCGCACCGCTTGCCGGTCAGGAATGGC
ATGATGCGCCGCCGCCCCGCGCCCGCAAGGCGCGTGTGGAACAGGCCGCGCCGTCGCCGAAGCCCGGCCCG
CGCGCCCAGCGCGAGGCGCAGCCTTCCTTCCTTAAGGATAATGGTATCTTTGAAATGCCGTCGCTGCATTT
CCTGGCCGAACCGAAGCTCGTGCAGCGCGACCCCGCGCTTTCCAAGGATGCGCTGGAGCAGAATGCCCGTC
TGCTTGAGGGCGTGCTGGAAGATTTCGGTGTGCGTGGCGAGATCATCAATGTCAAACCCGGCCCCGTGGTC
ACGCTTTATGAGCTTGAACCGGCACCGGGCATCAAGTCCTCGCGCGTCATCGGCCTTGCCGACGATATTGC
GCGCTCGATGAGCGCGATTGCCGCGCGTGTGGCTGTCATTCCGGGGCGCAACGCGATCGGTATCGAACTGC
CGAACCCCAAGCGCGAAATGGTCTATCTGCGTGAGATGCTGGCGAGCCGCGACTTTGAGCAATCGAAGGCG
AAGCTTGCGCTGGCACTCGGCAAGACCATCAATGGCGAGCCGGTCATTGCCGATATTGCCAAGATGCCGCA
CGTTCTGGTTGCGGGCACCACCGGCTCGGGCAAGTCGGTTGCGATCAACACCATGATCCTGTCGTTGCTCT
ATCGCATGACGCCGCAGGAATGCCGCCTCATCATGATCGACCCCAAGATGCTGGAGCTTTCCGTCTATGAC
GGCATTCCGCATCTGCTGACGCCGGTTGTGACGGACCCGAAGAAGGCCGTTGTCGCGCTCAAATGGACCGT
GCGCGAAATGGAAGACCGCTATCGCAAGATGTCGAAGGTCGGCGTGCGCAATATAGACGGCTTCAACCAGC
GTGTCGGTCTGGCGCAGAAAAAGGGCGAGCCGATTGCGCGCACGGTACAGACCGGCTTCGACCGCAATACC
GGCGAGGCGATCTACGAGACGGAAGAACTCGATCTCGAACCCATGCCCTATATCGTCGTGATTATCGACGA
AATGGCCGACCTCATGATGGTCGCAGGCAAGGATATTGAAGGCGCGGTGCAGCGTCTGGCGCAGATGGCGC
GTGCGGCAGGCATTCACGTCATCATGGCGACGCAGCGCCCGTCGGTCGATGTCATCACCGGCACGATCAAG
GCCAATTTCCCGACCCGCATTTCCTTCCAGGTGACGTCGAAGATCGACAGCCGAACCATTCTGGGCGAACA
GGGTGCCGAACAGCTTCTTGGCCAGGGCGACATGCTGTTCATGGCGGGCGGCGGGCGCATCCAGCGCGTGC
ATGGGCCATTTGTCGGCGACGACGAGGTGGAGCGCATCGTGCAGCATCTCAAGCTTCAGGGCGTGCCGGAA
TATCTCGATGCCATCACCGAGGATGAAGACGATGACGAGGGCGGTAGCGGCCCGGCTGGAACCGGCAATCT
GGAGGATTCCGACGATCCGTATGATCAGGCCGTGGCCGTCGTGCTGCGCGACAAGAAGGCGTCCACATCCT
ATATTCAGCGCCGTCTCGGCATCGGCTATAATCGCGCTGCCTCAATCATCGAACGCATGGAAGATGAGGGC
ATTGTCGGCCCGGCCAACCATGCGGGCAAGCGCGAAATACTGGTGCCCACGGGCGATGATGATTTC
>BR2097
ATGACCGCAAGCCAAGATTTTGTTGTCAAGGATATTAGCCTGGCCGATTGGGGCCGCAAGGAACTCGACAT
CGCAGAAACCGAAATGCCGGGCCTGATGGCTGCCCGCGAGGAATTCGGCAAGTCGCAGCCGCTGAAGGGCG
CGCGCATTTCCGGCTCGCTGCACATGACCATTCAAACTGCCGTTCTCATTGAGACGCTGAAGGTTCTGGGT
GCCGAAGTGCGCTGGGCTTCCTGCAACATTTTCTCCACGCAGGATCACGCGGCGGCGGCGATTGCGGCAAC
CGGCACCCCGGTTTTCGCCGTCAAGGGCGAGACGCTTGAAGAATACTGGACCTATACCGACCAGATCTTCC
AGTGGCCGGATGGCGAGCCGTCCAACATGATCCTCGATGATGGCGGCGACGCCACCATGTATATTCTCATC
GGCGCGCGCGCCGAGGCTGGCGAGGACGTCCTGTCCAATCCGCAGAGCGAGGAAGAAGAAGTTCTTTTCGC
GCAGATCAAGAAGCGCATGGCCGCAACGCCGGGCTTCTTCACGAAGCAGCGCGCCGCAATCAAGGGCGTGA
CGGAAGAAACGACCACCGGCGTCAACCGCCTCTATCAGTTGCAGAAGAAGGGTCTCCTGCCCTTCCCCGCC
ATCAACGTCAATGACAGTGTGACCAAGTCGAAGTTCGACAACAAGTATGGCTGCAAGGAATCGCTGGTGGA
TGGCATTCGTCGCGGCACCGACGTCATGATGGCCGGCAAGGTTGCCGTCGTCTGCGGCTATGGTGATGTCG
GCAAAGGCTCTGCCCAGTCGCTCGCCGGTGCCGGCGCTCGCGTCAAGGTCACGGAAGTCGATCCGATCTGC
GCGCTTCAGGCCGCGATGGACGGCTTCGAGGTCGTGACCCTTGATGATGCCGCCTCCACCGCCGATATCGT
CGTGACGACCACCGGCAACAAGGACGTCATCACGATCGACCATATGCGCAAGATGAAAGACATGTGCATCG
TCGGCAATATCGGCCACTTCGACAATGAAATTCAGGTCGCAGCCCTGCGCAACCTGAAATGGACCAATGTG
AAGCCGCAGGTCGATCTGATCGAGTTCCCCGACGGCAAGCGCCTCATCCTTCTTTCGGAAGGCCGCCTGCT
CAATCTGGGCAATGCGACGGGCCATCCAAGCTTTGTCATGTCGGCTTCCTTCACCAATCAGGTGTTGGGCC
AGATCGAACTTTTCACCCGCACCGATGCCTATAAGAACGAAGTCTATGTGCTGCCGAAGCACCTTGATGAA
AAGGTCGCTCGTCTTCATCTCGACAAGCTTGGCGCAAAGCTGACGGTACTTTCCGAGGAACAGGCTGCCTA
TATCGGCGTCACTCCACAGGGTCCGTTCAAGTCGGAACACTACAGGTAT
>BR2091
ATGGCGCAAACCATCGGCCTTTTTCAGATGCCAAGACTGGAGGAATTGGCGATCTCAAATCTTTATCGGCA
GCCTGCTCAGGCGCGGCCTGCACCCCATGTGCCTAATTGGTCAGCGCGAACGCATGTTGCGCAGCGTATCA
CGATCGAATGCAACCTGACGCACACGCTGGCGCTTGCTCGCGTTCTCTTCGAGAAGAAGTATGATTGCCGA
GGCAGCCATTGCGACCATCATGGA
>BR2088
ATGCGCTCCTCCGGCAGATCGAAATCGAAAAGATCAACACGCATGACTTACCTCGTATGGGCATTTCCAGC
GAAAGCGCGAAGCAATATCGCGTTTGGAAACGCAACGCATAAAGTCGGCCCCGGCCGAAGCCGGAGCCGAG
ATTTCTCGATACAAACTATTGAATCAGATGTCGGCGGCGATACGCGCGGTCACGATCTTGTCCGGATCGGA
AACCGGCTCGCCACGCTTGATCTGGTCGACATTATCCATGCCTTCGATGACCTGACCCCAGACGGTGTACT
GGCGGTCGAGCCATGGAGCATCGGCAAAGCAGATGAAGAACTGCGAATTGGCCGAATTCGGATTGGCGGAG
CGCGCCATGGAGGCCGTACCGCGCTTGTGCGGCGTGTTGGAAAATTCAGCCTTCAGGTCCGCCTTGTCCGA
ACCGCCCATGCCAGCGCGCGAACCGTCGAAATGAGCGCCACCCGACTTGCCGAACTTCACGTCGCCGGTCT
GCGCCATGAAACCGTCGATCACGCGATGGAAGACAACGCCGTCATAAGCGCCTTCGCGTGCCAGTTCCTTG
ATGCGCGCGACATGGCCCGGTGCGAGATCCGGGTAGAGCTCCAGCACCACATTGCCCTTTGTGGTTTCGAG
AACGAGCGTATTTTCTGGGTCTTTATAAGCCATTGGGCCTCTCCTGTTGATTTGCACCCCGAAAGTTCAAC
CGTTTCCGGGGCGCCAGATGGGTGTTACTTCGTGTCAGCTTCGATGGTGGCCTTGATGATTTTGTCGGGAT
TCTTCACGGCACCATTCTCGGCTTCGCTGCCCTTCTTAATCTTGTCCACGGCATCCATGCCGCTGACAACC
TTGCCCACGACCGTATATTGTCCATTGAGGAACGGGCCGTCATCAAACATGATGAAAAACTGTGAATTGGC
CGAATTCGGATTCTGGCTGCGCGCCATGCCAACCGTGCCGCGCACGAAAGGTTCCTTGGAAAATTCCGCCG
GGAGATCGGGG
>BR2072
GTGCTCGAAAACCGATATGCTCATCTTTTAAATATCGAAAAAGACCGTTTCTTTTTCGCCCTGAAGGTGGA
TATCGAAGTGATAGACGTCGCCCTCGCGCTGGCCGATCAAGGTTGGCACGCGCATACGATGCTCTATGCGC
GCCAATACCGGATCTTCGGCATTGGCGGCTTCTTCGTCGGAAAAATACATCCGCGTTTGCAGGCCGAGGTT
AATGCCGCGCGCTACGATCCACAAGGTTACATGTGGCGCCATGAGCCGCCCGTCCCGGAAGGGAACGCGGC
CGGGCTTGATCGTCTCGAAGATGAATTCGCCCGTTTCCATATTGCTGGGGCAGCGGCCCCAGCCACGAAAA
TGCGGATCGGCCTtGCCAcGGCTTTCGCTCGGGCTGTTATAGAGCCCGGCAGCATCGGCCTGCcAGaTTTC
CACCAGCGCATCGCGCAGCGGCGCGCCCATGCCGTCGATAACGCgCCCCTTGATAGTGATGCGTTCGCCAT
GCGCCTTGTCGTTATAAAGCGGACCTGTACCCAGATCTTCTGCAAAAACTCCGTTAATGCCGACGAAATTG
GGCGTAAGGCCGATATGGACGTAAGGACCGGCTGTCTGGGAAGCGGTCTCCTTGAGATAACCGAGCGGCTG
TACCATGTCAGTTGCCCTCCGGGCGGTTTTCAAAGAAGGTCGAACGGCGTCCGCGCAATACGATATCGAAC
T
>BR2059
TTGCCAATTGAGACGATCTTGCGTTCCGTCCTTGCGCGCAATGGTGCCATCGAACTTTTTTCGCGCCAGCG
CAACATCGCCGGTAATATCGATTACGCTCAAATCCGTTGCCCGGAAAATGCCTTCAGCCAGAGGCTCGGCA
TATTCGATAGCCGCACTTTCCGCAGCCTGGCGCAGCCATTCGTCGCGATAGCTGGCAAGCGTGGGGAAGCC
TGCATCCCATTTATCCGGGTCGGGGTTGTTATGGGCATGAATGCCGAGGAAGCGCGCCTCATCGAAATCAT
CCGCCACCATGGACCAGTCCTGCGCGATGAATGCCTCGATATCGCGGCGCACGAACATTTCCCATATTTGC
TCGCGGTCCTTGTCGCCTGCGAACGGATTAACCGCTCCCTTCATTCTATCCTCCTGATTATCTTAATTTTC
CGCGTGCGGCGACGGGCAGGATATCGA
>BR2047
ATGGCGCGGCTCTTAGCACAGAATAACCCGCTCGTCATCCCGCATGAAATCTGGCGTGTCGTGCAAGATTG
CGGGGATGGGCGCAAAAGAAAAAAGCCGGGCACGAAGCCCGGCTTTGAAGTTCTGCCTGATCTTCTGGATT
ATTTGCACTTCTGGATCGCGGCCAGTGCCGCGGAAATGCCCTTCAGGGAA
>BR2009
ATGACAGGAGAGGACATGCAAGCGATTATCGACGCCGAACAGAGTTTCAAGTTTCCGGTTCTCGTCGGCGA
TATCGGCGGCACCAATGCCCGTTTTTCTATCCTCGTGGATTCaAACGCAGAGcCGAAGGAGtTTCCCGTGC
TCCAGACGGCGGATTATGCCACAATAGACGAAGCGATCCAGCACGCCATTCTGGATCAGACGGCCATCCAG
CCGCGCTCTGTCATTCTGGCCGTGGCAGGCCCGGTGGACGGCGACGAGATCGACCtCACCAATTGCGACTG
GGTCGTGCGTCCTAAAAAGATGATCGCTGATCTGGGCTTTGAAGACGTGACCGTCCTCAATGATTTCGAGG
CGCAGGCCCTTGCCGTGGTTTCGCTGGAAGGCCACCATATGGAACAGATCGGCGGCAAACCGGAGGAGGCT
GTTGCCACCCGCGTCGTGCTCGGCCCCGGCACGGGCCTTGGCGTGGCAGGTCTGGTTCGCACACGTCATGC
ATGGGTTCCGGTTCCCGGTGAAGGCGGTCATATCGATATCGGTCCACGCACCGAACGCGACTACCAGATTT
TCCCGCATATCGAACGCATCGAAGGGCGTGTCACCGGCGAGCAAATTCTTAGCGGGCGGGGCCTGCGCAAC
CTCTATCTGGGCATCTGCGCCGCCGACAAGATCACGCCCACCCTTGAGACGCCAGTAGACATTACATCCGC
CGGACTGGACGGCAGCAATCCACAAGCCGCAGAAACGCTTGACCTCTTCGCCACCTATCTGGGGCGGCTTG
CGGGCGACCTTGCGCTCATTTTCATGGCGCATGGCGGCGTTTATCTTTCGGGTGGCATCCCGGTGCGCATC
CTTTCCGCCCTCAAGGCCGGTTCGTTCCGCGCAGCCTTCGAGGACAAGGCCCCGCACAAGGCCATCATGCG
CGACATACCGGTCCGCGTTATCACATATCAACTGGCGGCCTTAACCGGGCTTtCCGCTTTCGCCCGCACCC
CyTCGCGCTTTGAAGTTTCGACCGAGGGCCGCCGCTGGCGCATGCGCCGC
>BR2002
TTGCCGATTGTGCTGGCAAGCACGATCCGGCTTTTGTTTGCTCAATTCATCCGATTGAAACAGATCAAAGC
ATCTCGCATGAATATGGAAAAACGCGACGCATTT
>BR1982
ATGGCAACACCGTTCCGCAATTGCAAAACCCCGCCGAAGAATCCATCGAAATCGGCCTTTCCACCGaAAAC
CATCGCCATCACATCGAATTTCGGCGGCACGGGGCTCACGATTTTCGGGGCGCTCGACAATGCCGATCCGA
TGATCCAGCGGCAGGGGCGCTACGATATCATTGTCGTGCTGCAAGGCCCTGCGCGCGATATTGTGGTGCGC
AAGAAAGAACGCTATCTCGGCGTCTGGGTGAATGCCGCTTCCGAAACCTTTCTCGGCGTCCCGCTCTCCTA
TTCGCTGGCCTCCACCCGCAATTTGCAGGACATTGCAGATGAGAAAACCTATCGCCAGCTTTCGGTCGGCG
TGCATAATTTCTATCTGGTGCCGGACGACCCCGACAGCCCGTCCGATAATATTGTCAAATTCGGCAACGAG
CTGCGCGAAATAAAAATCCGGCAGGGGCTATATAGTCAGCGCATTGGCGGTGTGGAATTCATCTCACGCAC
GCTTTTCCGCGCCACGCTCAACCTGCCCGCCAATGTGCCCGTGGGCCGCCACAAGGCGCGTGCGCTTTTGT
TCCGCAATGGTGTTTTCCTGCGGGAAGCCAATGCAAGTCTGGAAATCGTCAAGGCCGGTCTTGAACAAAGC
GTCTATAACGCCGCCCACCAGAACAGCCTTCTCTACGGCCTTTTTGCTGTTTTCCTCGCCATCTTCACCGG
TTGGTTCGGGCGCATTCTGTTCCGCAAGGAT
>BR1948
ATGCTGATTGTCGAGTCGCATCGCGGCAATTGCAATGCCTATAATGCGGGCTGGCTTACGGCCaTGACCTG
TTTTCCGAACTTCAAATATATCCTCGTCATCGATGATGATGAACTGGCCGATCCGATGTGGATCGAGAACA
TGGTTTCCGCGGCCGGGCGGTTCGATGCAAGCCTCGTCGGAGGCCCGCAATATCCGGTCTTTGAAAAGCCG
AATTCGCAACAATGGGCCAAACACCCGGTTTTTATGCCGCCTTATACAAAGACAGGCGCTGTTCCGATCAT
CTATTCGTCGGGAAACCTGCTGATCGCGCGTCCTGTGCTGGAGGCCATCGGCTATCCGTTCCTGGACCTGA
TGTTCAATTTTACCGGCGGCGGCGATTCCGATTTCGTGGGCCGCTCCAAGGCGAAAGGTTTCCGGATTGCC
TGGTGCGCGGAAGGTATCGTGCGTGAAACCATTCCGGCGCGCCGTCTGGAAGGCGACTGGATCAGGGCGCG
GGGCTTGCGCAACGGCGTTCTTTCCACTCTTACTGAACAGCGTCGGCGCAAGGATGAGCCTTGCGGGCAGA
TACGTGTGTTCCTGAAGAGCCTTGCGCTATTGGCTTATTCGCCGATCAAGGCCTTGCGGCGCGGTCTTGCG
GCAGGCTTTGCGCCGGCTGGAATGTATTTTATCTATGTTGGATTGGGACGGGTTTTGGCACATTTTGGATA
TCTGAATGAGCAATATCGTAACCCGGACAAGAAC
>BR1945
ATGACGCAAAACGAACGTATAGGCGGAGATTTTCGTTGGGGCATATGGGGCACGGACATGATTGCCTCTAG
CTTTGCCGCCGACATTGCCGCAAGCGCCGGAATGCGCGTTGTTGCGGTTTGTTCTCACACGATGCAAACGG
CACAAGCTTTTTGCAGGCATATCGGCATTAACAAGGCTTTCGgCaATCCGCATGCGTTTCTGGCCGATCCC
GAAATCGATGCCGTCTATATCGCCTCGCCCAATATGCTGCATGTGGAGCAAGCCCTGGATGCCATAATGGC
GGGGAAAGCCTGCCTCATCGAAAAGCCGCTATCGCTTGATCCAGAAGGCGCGCGCCAGATCGAAACGGCAA
GCAAGGCGCGAAACATCTTTGCCATGGAGGCAATGTGGACCCGTTTCCTTCCCGCGGTGCAAGCGGTGAAG
CACGCTATCGATGCAAGCCGCATCGGCACGGTCACGCATATTGAAGCCGATCTTTCCTATAGCCGCGCCTA
TGACCCGGAAAGCCGCTTTTTCAGCCCGGCGCTTGGTGGCGGAGCCGCTTTTGATCTTGGCGTTTATCCCC
TTTCGCTGGCGCTTTATTTTCTGGGCCTGCCAGAAAAGGTTGATGGACATTGCCAGCGCGCGGCAAGCGGC
GTCGATTTGCGCAGTGAATTCAACCTCGGCTGGCCCAGCGCAACCGCCTGGATTTCCTGCGGCTTCGACAG
GGACGGGGAAAACCGGATGTTGATCGAGGGAACTGACGGCGCAATCCTGATCCACCCGCCCTTCCTGAAAG
CGCAAAGGCTGACATTCTTTTCGCGTTCGGCCCTGCACTCGCCCTTCGGCCCGAAAAACAGCAAGGGCCGC
ATCGGTAACATCATCAATCGCCTGCCGCTGCCCGGCCGCACAATCGAAACTCATGCCTTTGCAGGCAACGG
GCTTCAATTTCAGGCACAAGCCGTGCGCGATGCCATCCGGCGCGGGGAAATTTCCACCCCCATCATGCCGC
TGGCACAGAGCGCGGCGGTCGCCGATATCATCAGCAGGGTTTTGGTATCGGGT
>BR1937
ATGGCTGTCCGCCGCATCGTTGCCAATATTTCCGCCAGTGATCCTGCGGCAGCAAGGCGCTTCTATGGCGA
CTTGCTGGGCCTTGATGTGGCGATGGATTTCGGTTGGATCGTCACCTATGCAGGCGATGCCGAGGCGCGGG
TACAGGTAAGCGTCATGAGCGAGGGCGGCGCGGGAACGCCGGTGCCCGATCTCTCCATTGAGGTGGATGAT
GTCGATGCTGTGCTGGAGCGCGCCATCGCGCTTGGATTTTCCATCGAATATGGTCCGGCTGATGAGGAATG
GGGCGTGCGTCGTTTCTACACGCGCGACTCTTATGGGCGCCTCGTGAATATTCTGACCCACAAGCAT
>BR1934
ATGTCCGTCGTCCTGCCGCCGGTCAGCTTTCAAACTATTTCAGGGCTTGGCCCCCAACTATTCTCGGTGCT
TGCGCTGTGGGGCTGTTCGGTGCACAAATCGGGCATGAAAACAATTGTTCGTCCCCGTCGTTCCGTCCTGT
TTGTTCCTGCCGCAAATACGCGGGCATTGGAAAAATCGCTGACGCTTTCCGCCGATTGCGTGATCTATGAT
CTGGAGGATTCGGTTGCGCCGGAGGCGAAGGCTGCGGCCCGCGAGGCACTTGCAGCGCATCTTTCGGCCCA
TCCGAAAGTTGCTTTCGAGCGGATCGTGCGGGTCAATGCGGCAGACACCCCATGGGGCAGGGACGATGTTG
CGGCTGTGGCGAAAATGGCTGTAAACGCCGTCCTTTTGCCGAAGGTGGAGCGCCCGCAGGATGTGATCGAG
GCCGCCAATCATCTCGACCGGCAGGATGCCGATCCCGCCATGGGCGTATGGGCCATGATCGAAACACCGCG
CGGCATTTTGAATGTCGATGAGATTGCTGTTCTGGGGCATCGCTCGGCGGTGCGGCTTGCCTGTTTTGTGG
TAGGG
>BR1930
ATGCCAATTCTTTACACGACCCAGTCCACGGCAACCGGTGGCCGCACGGGCAGCGCCAAGACCGCCGATGG
GCGCCTCAGCGTCGTTCTCGACACGCCGAAGGAACTTGGCGGGCAGGGCGGTGAAGGCACCAATCCCGAAC
AGCTTTTTGCTTCCGGCTATGCTGCCTGCTTTCTCGGGGCGCTGAAATTCGCGGCGGCGAAAGAAAAGATT
TCGATCCCCGCTGAAAGCACCGTGACCGCGACAGTTGGCATCGGCCCGCGCGAGGATGGCACCGGTTTCGG
CCTTGACGTGGCGCTGTCAATTGCTCTTCCTGGCATCGACAAGGCAAAGGCGGAGGAGCTGGTGCAGGCCG
CCCACATCGTTTGCCCCTATTCGCACGCGACGCGGGGCAATCTCGACGTTCGCCTGAGCGTAGCC
>BR1893
GTGAAGGCGATTTTTCAAACGGCGCGGCTGGCTCTTGCTGGTGGCTTTCTGCTGGCAGCAATGGCTGGCGC
CGGTGCGGCAGACCGCATTGCTTTTGTCGTGCCTTCGGCGACCGTCTATCCCGGACAAATCGTGAGCGATA
CCGCGCTTCTGGAAAAGCAGTTCTTCATCAATGCGCCTGCGGCGACCCAATATGTGCTGTCGCTGGATCAG
GCGGTGGGCAAGGTGGCGCGCAAGACGCTGCTTCCCGGCAAGCCGATCCTCGTTTCGGCACTGGGCGAGCC
GTCTCTGGTCACGCGCGGCATTCCTGCACCACTGGTCTTTACCGCCGGCACGCTGACTATAACTGCGATGG
GTACACCGCTCGAATCCGGTGTGGCTGGCGATTTCATCAAGGTTCGTAACGTCGATAGCGGCATCATCGTG
TCCGGCACAGTGCTCGCAAATGGTCGTATTCAGGTGGGGATGCAG
>BR1873
ATGAAACTGATGGAATCCGCGCCTTTTCTGCTGATTGCCACGGGCACGCTGCTTGGCCTCATATTGCCGCT
CGGCAAGATCGCCGCACAGGCGGGAATACCGCCGGAAATGTGGACTTTTCTCTTTTCCGCAAGTGCGGGCG
TCATCCTTTTTGCCGTTCTGTGCCTGCGTAAAAAGAGGCTCGGCTTTTCCGGCGGGAGGCTTCGTTATTAT
ATCTGCACAGCCCTGATTTCCTATGCCTTGCCGAACTTCCTGATCCTGTCGGCGATCCCGCATCTTGGCGC
GGGCTTCACGGGTATCATGTACACGCTGTCGCCTGTCATCACGCTGCTTCTTTCCATGGGTTTCGGCCTGC
GCCGCCCGAATGCACTGGGCATCGGCGGTATTGCCATCGGTTTCGTCGGGGCGGCCATGGTGGCGATGACG
CGGGGGGAGGTCGGCAAGCCGACCGATCTCCTGTGGATTGCAGCAGCACTTCTGATCCCCGTTCTTTTGGC
TATCGGGAATATTTATCGCACGCTGAACTGGCCGAAAAACTCCGAGCCGACAGAGCTTGCGGCGGGCAGCC
ACCTGGCTTCGGCATTCATCCTTTTTATCTGCATCACTCTCTTCACCGGGCGTTTTCCGCTGGAAAGCTTT
GCGCTTGCACCCTTTGCAGCACTTGCGCAATCCCTGGCTGCGGCAGGCATGTTCGCGCTGTTCTTCCGGCT
GCAGGCGGTGGGTGGGCCGGTTTATCTCAGCCAGATCGGCTATGTCGCGGCGGCGCTCGGCCTTGTTTCAG
GCACATTGTTTCTGGGCGAGCATTATCCGCCGCTGACCTGGATTGGCGCGGCTGTGATCGCGGCTGGCGTG
GCCATGACCACGCGCGCGCAGAAAGGA
>BR1870
ATGGCGCTGGAACCGGCTGCTGCTGCCGCCGCCGAGCGCGCCACCAGCGACGAGATCGTGGAGCTTTATGC
ACTTGCCGCCAAATTCGACAATCCCGACCACACGCCGGAATCCATCGCCAAGGTCGATCTGGAATTCCATC
TTGCCATTGCCCGCATGTCCGGCAATCCTTTCATGCGTTCGGTCAGCGGCTTGATCGAGGCGGCACTGGCC
GTTTCCTTCCAGCTTTCCTCGCCCGCCGCCTCGCAGGAGGGGATCGCCGAATGCGCGGCCAATCACCTGCG
CATCGCCCATGCCATTGCATCACGCGATCCACAGAAGGCGCGCCTGGCCATGGAAAGCGTCATCAAGCTTG
GCGTGGAGCGCATCCGCGACGTAATC
>BR1869
ATGGCAGGAGCGGGCATGAAACCCAAAATATCCTTTAATAATGTGGTGATGCGCTATGGCGGTTTTCTTGC
TCTGGATCGGCTGAACCTTGATATTGCCGACGGTGAATTTGTGACGGTGGTCGGGCCTTCCGGTTGCGGAA
AATCGACGGCAATGAATATTGCAGCCGGACTTTTGCAGCCATCAGGCGGTGAGATACTTGTGGGCGACAAG
CCGGTCACGGGCCCCGGGCCGGAGCGCGGCGTCATCTTTCAGCAATATGCACTGTTTCCCTGGCTTACCGT
GCGTCAGAACGTTGAATTCGGCCTTAGCGTCGCGGGCATGTCGCGCGTCAAGCGGCGTGAAATCTCTGATC
ACTACCTGTCTCTGGTCGGCCTTACAGATTTCGCCGATGCTTTACCAAAAGCCTTGTCAGGCGGCATGAAG
CAGCGTTGCGCCATCGCGCGCGCCTATGCGGCAGCACCTGAAATCCTGTTGATGGACGAACCGTTTGGTGC
GCTTGACGCACTGACGCGTGTGCATATGCAGGACCAGTTGCTGGACGCCTGGAGCCGCGAGCGGCGCACGG
TGATGTTCATCACGCATGATGTGGATGAGGCGGTTTATCTCGCAAATCGCGTTATCGTCATGGCCGCCCGC
CCCGGACGGCTGGATCAGATCATCCCGGTCGATCTGCCATATCCGCGCACCGAGGCCATTCGTCTTTCCCC
GGAATTTGCAGCCATCCGCAACCGCGTCTGGCATGCTGTCTACCATCAGCAGCCCCAAACAGACCAGCAAT
CTTCACACGGTCAA
>BR1866
TTGTCTCCTGCGCTTTCTTCTGCCCACAGGAAATCCACGATGAACGTCCTCTATATCGACATTGACAGCCT
GCGCCGCGATCATCTGGGCTGCTACGGATATCACCGCAACACGTCACCGACTATCGACGCTATCGCGCGGG
AAGGCGTGCGGTTTGAGAATATCTATGTTTCGGATGTTCCCTGCCATCCGTCGCGCACGGCGCTGTGGTCG
GGCAGGCACGGTTTTCGCACCGGAGTGGTTGGCCATGGAGGCACGGCCTGCGAGCCTTTTCGGGAGGGGGC
AAGTCGCGCCTGGGCAGGCACCTTCTATGAAGAAGGCTGGATGAAAGCTCTTCGCGATCTGGACTATTACA
CCACCACCATATCCAGCTTCGGCGAGCGGCATGGCTGCTGGCATTGGTATGCCGGTTTCAACGAGGTCATG
AATTGCGGCAAGGGCGGGATGGAAAATGCTGATGAAATCGTGCCGATGGCCATAGACTGGATTGCGCGCAA
CAAGTCGCGCAAATGGTTCCTGCATGTCAATCTGTGGGATCCGCATACCCCTTATCGTGTGCCCGAGGAAT
GGGGCGATCCTTTCGCCGGTGAGCCGCTGCCCGCATGGATGACAGAGGAGGTTCTGGCGAGAAGCATCGCT
GGTTACGGTCCGCACAGCCCACAGGAACCGACCGGCTTTTCCGCGGACAACCCACATGCGCATTACACCAG
AATGCCAGCCCCGATAGATTCGATGGAGAAAGTTGCGGCCTGGTTCAACGGTTATGATGCCGGTGTTCGCT
ACGCAGACGAGCACATCGGCCATCTGATCGCGGCGCTGAAGGAGCATGGTCTTTACGACAATACGATCATT
GTGATTGGAGCCGATCATGGCGAAAATCTGGGTGAGCTGAATGTCTGGGGAGATCATCAGACGGCGGATGA
ATTCACCTGCAATGTGCCGCTTATCATTCGCTGGCCGGGCCAGGCTCCGGGCGTAAATCATGGGCTGCACT
ACCATTTCGACTGGCCTGCAACATTGATTGACGGGCTTGGCGGAAAAGTACCGTCGGTTTGGGATGGAAAG
TCATTCGCACCGGCAATGCGCCGGAACGAGAATGGCGGGCGCGATTTCCTCGTGCTTAGTCAGGGTGCCTG
GGCCGTGCAGCGCGGTGTGCGGTTCCGTCATCAGAATGAAGATTGGCTGATGCTTCGCACATGGCATGACG
GGCTGAAGGATTTCGGGCCTGTCACCCTGTTCAATCTCACATCCGATCCGCATGAGCAGACGGATCTATCC
CAAAGCCGCCCCGACATTGTCGCACATGCCAGCCGTATGCTCGATGAATGGTATGGCACAATGGCCCAATG
CAGCGATCAGGATGTCGATCCGCTCATGACGGTTCTGCGCGAGGGCGGCCCTTATCATACGCTGGGTGAGC
TTCCGGCCTATCTAGAACGGCTCCGCGCCACGGGACGGGGAGACCATGCCGACGCTCTTGCCGCGCGTCAT
CCGCAGCGTGAGAAGGAAAAGGCAACTTTGCGA
>BR1826
TTGATGTCCGAATATCTTTTGGAAATGCGAAACATCGGTAAAGAGTTCAACGGCGTCAAGGCGCTGGATGG
CATTTACCTAAAGTGCGCGCGGGAGAATGTGTCGAGCTGTGCGGTGAAAACGGGGCGGGCAAGTCCACCCT
GATGAAAGTGCCGTCGGGTGTTTATCCCCATGGCACATGGACTGGTGAAATTTTCTGGGAAGGCAAAGAAC
TTAAAGCCAGTGGCATTCGCGATACCGAAGCTGCGGGTATTGTCATCATCCATCAGGAACTGATGATGGTG
CCACACCTGTCGGTCGCGGAGAATATCTTCCTTGGTTGCGAGCCAACCACAGGCGGTTTCATTGATTATGA
CCAGATGAATGCACGGGCAGCCGAACTGCTCGCTCGGCTCAAGATAAACGATATCAATGTCGCATTGCCGG
TTTATCATTACTCAGGCGGCAAGCAGCAGTTGATCGAGATTGCAAAGGCAATCAACAAAAATGCCAAGCTT
CTGATCCTTGACGAGCCAACCTCGGCCCTGACCGCTTCAGAAACCCGGGTTCTCATCGACCTGATCAAGGA
CTTCAAAAAGCAGGGCATGGCTTGCGTTTACATCTCTCACAAACTCGATGAAGTGGCGGAAATCTCCGACA
CGGTAACGGTTATTCGTGATGGCGCGCATATCGCGACACGTCCGATGTCCGAACTGACAACGCCCGATATC
ATCACGATGATGGTCGGGCGTGAGATGAAGAACCTCTTCCCGCGTGAACCCCATGACATTGGCGAGGTGAT
GTTTGAAGCCCGCAATATAAGTTGCTGGGATGTGACCAATCCTGGCCGCAAGGTGGTCGACGATGTTTCGT
TTGCGCTAAGGCGCGGTGAGATTTTGGGCATTGCGGGCCTTGTGGGTGCCGGGCGTACAGAACTCGTATCG
AGCTTGTTTGGTGTCTGGCCGGGCGCCTGTCAGGGGCAGGTTTTTCTGGAAGGCAAGGAAATCAAAATCCG
CACGCCGCGCGATGCGGTGCGCCAAGGCATTTGCATGGTGCCGGAAGATCACAAACGCGATGGCATCCTGC
CGATCATGCCAGTTGGCCACAATATGACGATCTCCGTGCTTGATCGCTTTTCGCTACGAGGCCTCATCGAT
AAAGACGCGGAACTCGTGGCGATCCAGCGTGAGATTTTGAGACTCAAGGTCAAGACCGCCGATCCGATGCT
TGCCATTGCCTCGCTATCTGGCGGCAATCAGCAGAAGGCCGTGCTTTCAAAGATGATGCTGCCTGATCCGA
AAGTGCTCATTCTTGATGAGCCCACACGCGGCGTCGATGTCGGCGCGAAATATGAAATTTACAAGCTGATA
TTCGCGCTCGCCAGGCAGGGCGTATCGATTTTGATGGTTTCGTCGGAAaTGCCGGAAGTCCTTGGGATCAG
TGATCGTGTTCTCGTCATTGGCGAGGGCAAATTGCGCGGCGATTTCCCGAATGAAAATCTTACACAGGaGA
AGGTTCTTGCCGCTGCAATTGGCAAGCCTGCAACCAACGCGGCc
>BR1806
TTGCTGGCTTACATATCCGGGCGGTTGGTTTCGCTCCTGCTTACGACGCTTGCGGCCTCCGTCATCGTCTT
CCTGCTGATGCAGGTGCTGCCGGGCGATCCGGCGGCGGTGATCCTCGGCATTAATGCGCAGCCCGAGACGC
TGGCCGCATTGCACAAGCAACTCGGCCTCGATCAACCGCTCTGGTGGCGCTATCTTGAATGGATAGGCGGC
TTTCTCAAAGGCGACTTCGGCACGAGCTATACCTATTCCGTGCCGATCAGCGAATTGCTCGGGCCACGCAT
CATGGTCACGCTGCCGCTGGCGCTTCTGTCCATGGTGCTGTCGGTGGCCGTTGCCATTCCGGTTGGCGTCT
ATGCGGCTTCCAGGCGCGGCAAGACCGGCGACGTGCTTTCCATGGGCGTGGCGCAGGTCGGCGTTGCGATC
CCGAATTTCTGGCTTGGCCTGCTTTTGATCCTGTTGTTTGCCTTGCAGCTTGGCTGGTTTCCCGCTTCGGG
TTTTGCAGGCTGGGAAGGCGGTTTCTGGAATGGCATCCGCTCGCTGTTTTTGCCCGCATTGGCGCTTGCCC
TGCCGCTTGCCGCCATTCTGGCCCGCGTTACCCGATCCGCCGTCATTGAAACTCTGGGTGAGGATTTCGTG
CGCACAGCGCGCGCCAAGGGCCTGACGCGCAAGGCGGCGCTCTGGCGCCATGCCGTGCCCAATGCGCTGAT
CCCGGTCGTCACCATTATTGGCCTGCAATTCTCCTTTCTGCTGGCGGGAACCATCATCATCGAAAATGTCT
TCAATCTGCCCGGTCTCGGAAGGCTGGTATTTCAGGCCATTGCGCAGCGTGATCTCGTCACCGTGCAATCG
CTGGTGACGTTGCTTGCCGCTTCCGTCATCGGGGTCAATTTCATTGTCGATCTGGTCTATGGCTTTATCGA
TCCACGCCTTTCGACGGGAGGCAGCCGA
>BR1755
GTGGGGCCGCTTGCAACCGGCTTTTGGCGTCTGGCGCTGGCCGTACTGCCGCTGTTGCTCATGGATGGGCG
TGAGCGTCATGAGAGAAAGGATGCAGCGCCTCTCACGCTTGCAGATTGCCTGCGGCTCGCATTGCCCGGCT
TTTTCATTGCCGCCGATCTCGGCGCATGGCACCTGTCGCTCCATTCCACATCTGTCGCCAATGCGACCCTG
CTTGCAAATATGGCGCCCATTTTCGTCACCATCGGTGCATGGCTTCTGTTCCGCTCGCGCGTCAGTTCATC
CTTCATTGTCGGTCTTGTGGTGGCAGTTGCAGGCGTAATCGTGCTCAAGGGCGGCCCCGGCGAATTGCTAC
ACAGCCGCTCGTCCGGCGACGCCATGGCGCTTCTCGCCGCCGTTTTCTACGCGGGCTACATCCTCTCGGTC
GGCCGTTTGCGCAGCCGCTTTTCCACCATGCGCATCATGTTGTGGAGCAGCGCCATCGCGGCCATTTTCTC
GCTTCCGGTCGCCTATTTCATGGAAGGGCATATCCTGCCCGCAACGCTTTACGGCTGGCTTGTCCTCGCGG
GCCTTGCCTGGCTCACCCATGCAGCAGGCCAGAGCATGATTGCCTATGCGCTCGCCTTCCTGCCAGCCTCC
TTTTCCTCCCTCACGCTGCTTTTGCAGCCGGTCGTAGCGGCCTTCCTCGCCTGGACCATCCTGTCGGAGCC
GATCGGGCTGATGCAGGCCATTGGCGGTGTCATCGTCATTTCCGGCATATTGATCGCCCGACGAGGA
>BR1714
TTGCGCAAGCCGTACAGTCCATCGACAGCGGCGCAGCCCTTGCGGTTTTGCAAAAGGTCATTGCCGTTTCC
AACGACAAGCCGGCCTGACAGGAAGGATCGCGCCATGTCCACCGATATTCTTCGCAAGATCGAAGCCTATA
AGCGGGAAGAAATAGCCGCCGCCAAGGCGCGTCTTGCCCTTGATGAACTGAAGGCCCGGACGAGGGATCAG
TCAGCGCCGCGCGGTTTTCTGAAGGCACTTGAGGCCAAGCGCGCCGCCGGTCAATTTGCACTGATCGCGGA
GATAAAGAAAGCAAGCCCGTCCAAAGGCTTGATCCGCCCCGATTTCGACCCGCCAGCGCTTGCAAAGGCTT
ATGAAGAAGGCGGTGCGGCCTGTCTTTCCGTGCTGACCGACACGCCTTCCTTTCAGGGCGCGCCGGAATTC
CTCACGGCCGCACGCCAGGCTTGCAGCCTGCCCGCATTGCGCAAGGATTTCCTGTTCGACCCCTATCAGGT
CTATGAAGCGCGTAGCTGGGGAGCGGATTGCATTCTCATCATCATGGCCAGCGTCGATGACGATCTTGCAA
AAGAGCTGGAAGACACGGCTTTCGCACTGGGCATGGATGCGCTGATCGAAGTGCATGACGAAGCTGAAATG
GAACGCGCCCTGAAGCTTTCCTCGCGCCTGCTCGGCGTCAACAATCGCAATCTGCGCAGCTTCGAGGTCAA
TCTCGCCGTTTCCGAGCGGCTTGCGAAAATGGCTCCGTCCGACCGTCTGCTGGTTGGCGAAAGCGGCATCT
TCACGCATGAGGACTGCCTGCGGCTTGAAAAGTCCGGCATCGGCACTTTTCTGATAGGCGAAAGCCTTATG
CGACAACATGACGTTGCGGCAGCCACCCGCGCGCTTTTGACGGGTGCGGAAAAGCTA
>BR1695
GTGAACGAGGCTGATGCGCTTGATATCGTCAATTCGGCGATCTGGACCGTGCTGACGGCAAGCGGGCCGGC
GGTGCTTGCCGCCATGCTTGCGGGCATTGGGATTGCGCTGTTTCAGGCGCTGACGCAAATTCAGGAAATGA
CCCTGACCTTCGTGCCGAAAATCATCGTCATTTTCGTCGTCCTGGCGCTGACGGCACCTTTTGTCGGTGCG
CAGATCAATGCTTTCACGCTGCTTGCCTATTCCCGCATCGAAAAGGGTTTT
>BR1689
TTGCAGGGTAAAACTGACGTGATATTCCAGGCCCTATCTTTCGGAAGAGACCTACGGCCAACCGGTGCGCA
TTTGCCGGGATGGCGGAGTATCTGCGCGGTTATGGGACTGACGTTCACCGGCTGTGTTTTCATCCAGGGAT
CAGCCAGAGCGCAGGATGGCAACAAGGCAACTGCCGTGCTGGAGCTTTACACCAGCCAGGGCTGCAAATCC
TGCCCGCCCGCCGACAAGAATTTCGCCAAATATGCCGACGATCCCAATGTGCTGGCGCTTTCGTTCCACGT
CAATTATTGGGATTACATGGGCTGGCGCGATACGCTGGCCACCCAGGACAATACCGACCGGCAGAATTCCT
ATCGCAACGCTTTTCATGCCAAGATGATCTATACGCCGCAAGCCATCATCAATGGCGCAGTGGAAGCAAAC
GGACGCAATGGCGCGGCCATCAATGCGAAGATCGCCGCAAACAAGCTGGACGTGCCCGTTAGCATCCAGCA
GCTCAACGATGGACGCCTTTCCATCGATATCGGTGCAGGCAAGAAGCCCAGGGATCCGGTTCACGTGGTTC
TATTCTATTTCCGCGATTCGGCCACCATCCCCATCACCGAGGGTGAGAATGCCGGAAAAGCCGTCACCTAT
CGCAATACGGTGAGCGATATCGACACGATCGGCATGTGGGATGGAAAGCCGCTCAAGATCGAGCTTCCGGC
CTCCGAGCTGAAGCGCAAAGACGTGACGGGCTGTGCCGTGGTTCTGCAAGAAACGCGCAGCGACAATTCGC
TGGGACCGATGCATGGCGCCGCCATGTTCAAGCAGAAGCGTCCCGTCTCCTTC
>BR1679
ATGTTCAGCCGATTCTTCCGTAATGACCGACGCTCTTTCGATTCGCTTTCTGAACAGGAAATTCTGGCGCT
TGCCATATCCTCCGAGGAGGATGATGCCCGCATCTATCTGGCTTATGCCGATGGCCTGCGCGACGAGTTTC
CGCAATCGGCGAAGATTTACGAGCAGATGGCCGCAGAGGAACACGACCACCGCGCAGCACTGATTGAGCGG
CATAAGGCCCGCTTTGGCGACCGGATTCCGCTGATCCGCCGCGAGCATGTCAGTGGCTATTATGATCGCAA
GCCGGACTGGCTGGTGCGTCCGCTCGGCATCGACAAGGTGCGTGAGGCCGCCTCCGAGATGGAGGAACAGG
CTTATCGTTTCTATGTCGAGGCGGCCAAGCGCGTCAGCGATGCAGACACGCGCAAACTGCTGGGTGACCTT
GCCCAGCAGGAAAAACAGCACGAAGCCAAGGCTGAATCGCTGGAACACACGCTGACACCGGATAATGTGCA
GGATGAGGAACGCGCCGCCGAGCGCAAGCAATTCATCCTCACCTATATCCAGCCTGGCCTTGCGGGCCTGA
TGGACGGCTCCGTCTCGACACTCGCACCGATCTTTGCCGCCGCCTTTGCCACGCAGGATACATGGCAGACC
TTTCTGGTTGGCCTTTCCGCCTCGGTCGGCGCCGGTATTTCGATGGGCTTTACAGAAGCCGTGCATGACGA
CGGCAAGCTGTCGGGACGCGGCTCGCCGATCAAGCGCGGCATTTCATGCGGCGTCATGACGACGCTGGGCG
GGCTTGGTCACAGCCTGCCCTATCTGATCAAGGATTTCTGGACTGCGACAAGCATCGCCGTGATCCTTGTC
TTCTTCGAGCTCTGGGCCATTGCCTTCATTCAGAACCGCTATATGGAAACGCCCTTCTTCCGCGCCGCACT
CCAGGTCGTTTTTGGCGGTGCGCTCGTCCTGGCGGCGGGCATATTGATCGGCAGCGCC
>BR1671
GTGGAGCGACCCTACCAGTCGCGCACGCTGCGCACGCGTGATCCGCACGGCATTCCGCTTGAGTTCTATTC
CAGAATGGACCGCCTGCCGCCGATCCATCAAAAATATGCGCTCTATAAGGGCGTGAAGCCGCTGCGCATCG
ACCACTTCAACTGCTTCTCGCCTAATGTGGATGAATCCGTCGCCTTCTATAACGAGCTGGGTTTCCGCGTG
ACGGAATATACCGAGGATGCCGAAACTGGCCGCCTGTGGGCCGCCTGGACGCACCGCAAGGGCGGCGTTCA
CGATATCGCCTTCACCAATGGCAAGGGGCCGCGCCTGCACCATACCGCTTTCTGGGTGCCGACACCGCTCA
ACATCATCGACCTGCTCGACCTCATGTCCACCACCGGCTGGCTTGCCAATATCGAGCGCGGTCCCGGCCGC
CACGGCATTTCCAATGCCTTCTTCCTCTATGTGCGCGACCCGGATGGCCATCGCATCGAAATCTACTGCTC
GGATTACCAGACGGTCGATCCCGATCTGGAGCCGATCAAGTGGGATCTGAAAGACCCGCAGCGCCAGACGC
TCTGGGGTGCGCCTGCACCGAAATCCTGGTTCGAGGAAGGCAGCTTGTTTGCCAGTGTGCAGCCGGTCGAT
TCCGTGTTGAAGGCACAGCCGATCGTCGCGCCG
>BR1636
ATGAGCCCCCACGACAGCAACATCATTCTCGATGTACGCAACCTATCGGCTGGCTATGGTGAGCTGCCGGT
CATTCGTGATCTTAATCTTCATGTCAAGGAAGCGGAAATCGTAGCACTGGTTGGCAGCAACGGCGCTGGCA
AGACAACGTTTCTGCGCGCGCTTTCTCATCTGATCCATTCCGAGGGCCAAATCGAGTTTTGTGGCGAAGAC
GTTTCCAGATCGACATCGGATTATGTATTTCGACGCGGATTGGTACATATCCCCGAAGGGCGGCAACTCTT
CGACCGTATGTCGGTGGAAGAAAACTTGTTGATGGGCGCTTATCGCAGCAAGGACAAGGCCCGGACCAAGG
CTGATCTGGAAAAGGTCTATGTTCTATTTCCCCGCCTCGCCGAAAGGCGCAAGCAGTTGGCTGGCTCCATG
TCAGGCGGAGAGCAGCAGATGTGCGCCATGGCTCGGGCGCTGATGGCCAATCCCGTTTTACAGCTGGTTGA
TGAAATGAGCCTCGGCCTTGCGCCCGTGGTTGTCGACCAGTTGATGACGATCCTGCATGACATTCGCAAGT
CAGAGCAGGTAACCGTGCTTCTGGTCGAGCAGGATATTCATCTTGCTCTGGACGGCGCAGACCGTGGATAT
GTGATGGAAACCGGCACCTTCACGCTGGAAGGCAAGGCGCAGGATCTTATTCACGATGAGAGGATGCAAAA
AGCATATTTGGGTTTG
>BR1635
TTGCTTGGCTTTCTGTTTGATGGCGACGCCGAAGTTCATTTCTTTCGCAACGACTTCCACGGCGTTGGCTG
CATCATCGGCTGGCCGCCCTTTTTCGCGTTTCCCCTAGAAGAGCGGTTCCTGTTT
>BR1622
ATGTCGAAAGCACGCACTTCACCCGTTCGTACCGCACTCGTTCACGCGACCCTGATCTTCTATATGTTGAT
CGCCGTGTTTCCGGTGGCGCTGACGCTGATCAATTCCTTCAAGAGCCGCAATGCGATCTTCCGTGAGCCGT
TGCGGTTTCCTACGCCGGAAAGCTTTAGCCTCATCGGTTATGAAACCGTTCTGAAGCAAGGTTCGTTCCTG
ACCTATTTCCAGAACAGCGCAATTGTCACGGTTGTCAGTATTTTTCTCGTCATCCTGTTTGGCGCGATGGC
GGCCTTCGCGCTCGCCGAATATCGTTTCCGCGGCAATATGCTGACCGGGCTCTATCTGGCAATCGGCATTA
TGATCCCGATCCGGCTTGGCACGGTCGCACTGTTGCAGGGAATGGTTGCAGCAGGGCTGGTCAATACGCTG
ACGGCACTCATACTTGTCTATACGGCGCAAGGGTTGCCGCTTGCGATCTTCATTCTCTCCGAGTTCATGCG
AACCGTTTCGGATGACTTGAAAAATGCCGGGCGAATAGATGGCATGTCGGAATATGCGATCTTCTTCAAGC
TGGTGTTGCCGCTGATCCGTCCCGCCATGGCTACCGTCGCCGTCTTTACCATGATCCCGATCTGGAATGAT
CTCTGGTTCCCGCTCATCCTCGCGCCCAGCGAGGCCACCAAGACCGTTACGCTTGGCGCACAGCTTTTCAT
CGGTCAGTTCGTCACCAACTGGAATGCAGTCCTCGCGGCCCTGTCATTGGCGATGTTCCCGATCCTCATTC
TCTATGTTCTCTTCTCGCGGCAGTTGATCCGTGGCATTACGGCTGGAGCGGTAAAA
>BR1620
GTGAAGCGTATCAGGAACCGGAAACAGGCGCTGCTGATTGCGGCATTGCCGCTTCTTGTCTTTTGTGGCCC
GCCACAAGCCATGGCGCAGGATGTCAGTGCCGCGCGTCCGGCAGTGCTTCCCCTTCCCGGCGAAGCCGAAG
GAGCCAGTATCGGCTCCATTTCCGTTCGCCTGCGCCAATCCAGCGGCAATGCAGAACGCGACGCCGCCGCG
CAGGAAGCCGCACGCCGCATCACCAAATCCCTCGACGGATCGGAATTCAGCGCCATCGAAGCCGGCGAAAT
TATTGCCGAACTCATTCGTCAGGAAGCAATCTCCGACGCCAGATTTTATCTGCTCCCATCTGCCTCGCAAA
CCGGCGTCAAACTGGTTTTTGAAATCGACGCCACTTCAGGCACCAAGACGAAGCAAGAGCCAACCGGCCTT
TTTCTCGGCGACACATCCTCCTTTCCAGTCCTCTATGAAAGCAACCGCGCAGTACTGAGAACCATTATCGG
CGCCGGATTGGGCGGCTATTCCGATGTGAATGCATGGTTCGGAAGGCCGGATCTGTTCAATGGCAGGAATC
CGCTTGCTGGCAAATTGCCGGGCCGCTCAAACACATGGAACGAAGGCTATCTGGAGCTTGGCGTCGGCGGC
GCCGCGCAATTGGGCGAAAGCAACCTCTATGGGTTCGGCGCTGTCACCGGCATGTTTTCCTGGACCCTCGG
ACAGGATATTTTTCGCGATGACGATCGCGGCTTCCTCGACTTCGAGAAAGCCTATGCCGGTGTGCTCTATG
TATCGCCGAAGAACCGGAAAAACCATTTCAAATTCTCGGCCGGGCGGCAGGCTTATACACTCAATGACGGG
TTTCTGGTGAATGCCGTTCGTGGCTCCACCAGCGCCGGCGAACGCGGCGGGCTTTATCTCGGCCCCCGGCT
CACCAACGACTTTTCGGTTCTGGCGCAAGGTGCTTATGATCGCTGGAACTATAGTGCCTTCTTCATCGATC
CCAACGAAGTTGAATCGCTGGAATCCGACACCACTTTTCTCGGCGCAAATCTGCGTTACAACTTCACCGAC
AAGTTTTCTGCCGACACTACATTAATAACGATCCCCAACTCGAAAACACAATTTTCCACCCCCTCTGGCAG
ATTAATCCCGCGTGAAGGGCTGAACACGGTTTCAGGCCATTTGAAATGGACCAGTGCTTTCGAGATGCCCG
GCCTGTGGCTCGAAGGCGAACTGGCGCTCCAGTTTCACCCCGGTGAGGATGTGTCGGCCTGGGCCTATTAC
GGAACAGTCGGCTATCTCATGAAAGAGATTGGCTGGACGCCAAGTCTTTCCTATCGCTACGCCTATTTTTC
GGGCGACGATCCGAACTCATCGCGTTATGAGCGGTTTGATCCCCTGCTCTCGACCGGGCTTGGCATCTGGT
TGCAGGGTGTTTCGTTTGGAAAGATCACCACCAATTCCAATCTTGAGACGCACCGTGTCCAGTTCAATGTC
GCACCCGACGAGCGGCTGAACCTGACATTCGACTACTATCTGCTGCGTACCCCCCAATTGAACAATCTCGG
TTCAAACCCGGCGCTTTCGACGCTATCGTCGCACGATCTTGGGCAGGAACTTTCACTTTCAGCACGCTGGA
GCGCGACCAAAAGCCTTTACCTGCAAGCACTTGTTTCACATGCCATTCCCGGCACGGCGCTACGCGATATC
GGTGCCGACCGGAACTGGACGACTTTTCAGGTCAGCCTCTACGGGGGCTTT
>BR1532
TTGCTCTGGATCACGCTCGTCAGCGAACTGATATTCTCGCTCGATCCCTGTGCCCTATCGACAGTGCTGAG
CGTCGAATTGGTACTTTGGCACCCGGCGACGGTCAAAGCCAGAAGTGCGGCGGCGATGATAAGGCCGTTTC
GGCCTGCTCCACCCGACGCCTCAATTCCCGAAACCTCCGTTTCAGACAATTGCATCACCTTTAAGCGCATC
GCCAATTGCAGCATTCCTTTCAGCTCGACAGAAACAGCAAGGCCACGGGCCCCTGCTTCTTTTCCATTCCG
CACCGGATCTTCAGACCTTACGCCTCGCCGGACGATTTGTCAGTTCCGGCACTATGCTGGTTTGAACCAGC
AGATAAAAATAGCAAAGAGGCGGCAGGAAAAGAGATTACTCCGCCTGCGGACGCCCAACCTCAAAGCCAGG
CACCCTTTTTTGTTTTACACAAAA
>BR1531
ATGTTCTCAGGACATGGCATGGCCGGGCCGGGCCGGGCCGGGCCGGAGCATCCATCATGGAACAGGCCTCC
CGCTCAAGCGGCCTCGCCTCTATTTTCCACAATCGAAACGCGCAAAATCCGGCGCAAGCCCGAAGCCGCAC
CGGAAAGCAAAAAGGCGCCGCTACCACAGCGCCTATATCACAGCCAATCAGCGAGCGCCTGTACGGCCGCC
GATGAGACCTTCGCGCTGGGCGCGCTTACGGGCCAGCTTGCGTGCGCGGCGAACGGCTTCTGCCTTTTCGC
GGGCGCGCTTTTCGGACGGCTTTTCATAATGGCCACGCATCTTCATTTCGCGGAAGATACCTTCGCGCTGC
ATCTTTTTCTTGAGAGCGCGGAGAGCCTGATCAACATTATTATCGCGGACGAGTACCTGCACGTGTATTCC
CGTATCGGT
>BR1522
ATGCCCAAGGGGCTTTATGCGCGCTCGCTCATCATCATTATCGCGCCCATGGTGCTGCTCCAGTCGGTCAT
TGCCTATGTGTTCATGGAGCGCCACTGGCAGATGGTGACGGAACGCCTTTCGACGGCAGTGGTTCGCGATA
TCGCAGCAATCATCGATCTTCTTGAAACCTATCCGCAGGAGCCGGGTTACGACAATCTGATCCGCATCGCG
CAGCAGCGGCTGGCGTTGAACATTTCCATCCTGCCCCCCGATCCACTGCCGCCGCCGGGGCCGAAACCCTT
CTTCGCCATTCTCGACTATTTCCTGAGCGAAGAGATCACCCGGCAGATCAACCGCCCTTTCTGGATCGATA
CTGTGGGTGATTCCGATCTGATCGAAATCCGCATCAAGCTTGAAGACAAGGTATTGCGCGTTTTTGCCCGG
CGCAGCCAGGCCTATGCGTCCAATACCGGCATCTTCCTCACATGGATGGTGGGCACGGCGCTGGTTCTGCT
TATCATTGCCATCGCCTTCCTGCGCAACCAGATCAAGCCTATCCAGCAATTGGCGCAGGCCGCCGAAAGCT
TCGGCAAGGGGCGGCCCATGCCGGAAGGTTTTCACCCGCGCGGGGCGGAGGAAGTGCGCCGCGCTGGCCTC
GCCTTTATCCAGATGCGCTCGCGTATCGAACGCCAGATCGAGCAGCGCACCGCCATGCTTTCCGGTGTCAG
CCACGACCTGCGTACCATTCTCACCCGCTTCAAGCTGCAACTGGCCCTCGCCGGCCATTCCATCGATACCG
AGCCGCTCAATCAGGATATTGCCGACATGCAGACCATGCTGGAGGGGTATCTGGCCTTTGCGCGCGGTGAG
GGAGCAGAAGAAACCGCGACTTTCGACGTCACCCGGCTTTGCGAAAAACTGGAAAGCGAAGCGCGCCTGCG
CGAGCGCGGTTTCAAGTACACGATAGAGGGCGACCGCGAAATCAACGTGCGCCCGAACGCCTTCTCACGAC
TTGTCAGCAATCTGGTTTCAAACGCCTTCCGTCACGCGCAGAATGTCGAGCTTTCGATTTCGCATGTGGAC
GGCTGGCTTAAAGTCATCGTGGACGATGATGGACCGGGCATACCCGAAGATCGCAGGGAGGACGTCTTCAA
GCCTTTCGTGCGGCTTGATGAGGCACGAACGCAGGATTCAGGCGGCACCGGCGGGCTTGGCCTTGCGATTG
CCCGCGACATCGCACGCAGCCATGGCGGCGACATTGCGCTGGAGGATTCGCCGCTCGGCGGCCTGCGCGCC
ACCATTCATATTCCGGCT
>BR1507
ATGGCCGCGCATTATGCAGGCATTCCGTTTGAAAGCGTCATCGTCAACACATCCGCGGAGCCGGAAACGCT
CATTCAGGTCAACCCTTTGGGCAAGATTCCAACGCTTGTGACCGATGACGGCAAGGCCGTTTTCGATAGCC
GCGCCATCACGCAATATCTCAACCGCGTTTCGGGCAACAAGCTTTTTCCACGCAATGCCGAAAAGCGCACC
GATGCGGAACGCTATGAAGCAATCGCCGATGGGCTTTGCGACGTTTTGCTGGCCCATGTCTATGAACGGCG
GATGCGCCCGGAAGAGAAAATTCATCAACCCTGGCTCGACCTGCAATGGCGTAAGGCCGAACGTACGCTTG
ATCTTCTCAACGAGGCCCCGCCGCGCCTTGCCGGAAAGCTTCATGCGGGACATCTCGCGGTTGCCGCAGCA
CTCGGCTATCTGGCGTTGCGCTTTGAAGGCAAATGGGAACGCAACCGCTCGAAGCTGAAACGCTGGATGAA
GCGCTTTCAGGAATTGCACCCGGAGCTGGTCGAGCTTCTGCCGCGCGGC
>BR1503
GTGACTTTATCAGGGCAAGTTCCGGTTCGGGAAGTTGAGGTCGTTGCACCCAACTTCAAGCGCCGCCTTTC
CGGCGTGACCTCGACGATTGTTCAGCTCATTCCATTGCAGCGCGCAATGGGGCTGAAAATTGCCACTATGG
GGCCGGGCCTGCCTGACACTCTCCCGCATCTTGGATGGAGCGCGTTGCCCTCCTTCTGGTCGCGACCTAAA
ACCAGACGGTTTCGCATCTGGCATGCGCGCCGCAACATTGAAATGCTTGCCGGAATTTTCATGCGGGATGT
CCTGCGCATGAAACTGCGGCTCGTTTTTACCTCGGCCGCCCAGCGCCATCACAAACCTTTCACCAAATGGC
TCATCCGCCGCATGAATGCGGTGATCGCGACAAGTGTGCGTTCGGGAAGTTTTCTCGAAGTCCCGCATCAG
GTCATCATGCATGGTGTCGATCTGGAACGGTTTCACCCGCCTCTTGCGGAGGACGATGATTTTTCCGCTTC
CGGCCTGCCGGGTAAATATGCCGTGGGGTGTTTTGGACGGGTTCGGCCTTCAAAAGGAACGGACCTTTTCG
TTGATGCGATGATCGCGCTCCTGCCGAAATATCCTGACTGGACGGCCATTGTCACGGGGCGCACAACCGCC
GAATATCAGGCCTTTGAAGCCGAGCTGCGCACCAGGATTGCGGCAGCCGGTTTGCAGGACCGTATTCTTAT
TCTGGGCGAAGTGCCGGACGTGCGCGTCTGGTATCGCCGGCTCACGCTTTACGTCGCGCCTTCCCGCAATG
AAGGCTTTGGCCTGACGCCGCTTGAAGCCATGGCCTCGAAAACAGCCGTCGTTGCCAGTGATGCCGGGGCT
TATGCGGAAATGGTCGTTGAGGATACGGGCCGCTTTGTGCCAGCCGGTGACGGGCGCGCATTGACGAACGC
CATCGAACCTTATCTGGCCGATCCGGCCATGACAAAACGCTGCGGCGAAAATGCTCTGGCGCATGTTCGCG
AAGCCTTTCCGCTCCAAAAGGAAGCGGCTGCTATCAGCAGCGTTTATGAGCAGGTTTTCGCAGGAAGA
>BR1501
ATGGGAAACGCCCGACATTGCAGGAATTACCGAATAATGGCCACCCGCAAGACAACCACCGGCGAGCAAAC
GCATCTGCCGCCCGATGACGAACAGGACGTTGCGGGCATCATTATCGGTGAGGCAGAAACCGAAGACGATG
ACGATGCGGCGGCAAACGGCGAGGAAGAGCCTGCCCGCATAGCTTCGGCTGCGGAATCCATCCAGTGGGAT
TCGTCGGACGGGCTGCCCGATACGGCGGGGCTCAGCGGCCCGGACATCATCAATGCCTTTATGAAGCGCCT
GCCCAACAATCCCGGTGTCTATCGCATGTTCAACAGCGATGGAGACGTGCTTTATGTCGGCAAGGCGCGCA
ACCTCAAGAAGCGCGTTTCCAATTATGCGCGCGGCATTGGCCATTCCAATCGCATCACACGCATGATCCGT
GAAACCGTGACGATGGAATTCGTCGTCACGCGCACGGAAACGGAAGCACTTCTGCTTGAAGCAAATCTCAT
CAAAAGATCGCGGCCACGCTTCAACGTGCTGATGCGCGACGACAAGTCGTTTCCGTATATTCTGCTGACCG
GCGGCCACCGCGCGCCGGGCATTTTCAAGCATCGCGGCGCACGTTCGCGCAAGGGCGACTATTTCGGCCCC
TTTGCTTCGGCGGGGGCTGTGGGCCGCACCATCAACGCGCTCCAGCGCGCTTTTTTACTGCGCACCTGCAC
CGATTCCGTCTTTGAAACCCGCACACGGCCCTGCCTGCTCTATCAGATCAAACGCTGCTCTGCGCCCTGCA
CCTATGAAATCAGCGACGAGGACTATGCCGGGCTTGTGGCAGAGGCCAAGGCCTTTCTTTCCGGCAAGAGC
CAGTCGGTCAAGGATCATCTGGCAGCGGCAATGCAGGCAGCTTCCGCCGATCTCGATTTTGAACATGCCGC
AGTCTATCGCGACCGTCTCGCCGCGCTTTCACACGTCCAGTCCCATCAGGGTATCAATCCGCAAACGGTCG
AGGAAGCCGATGTTTTCGCCATCCATCAGGAAGGCGGCATGACCTGCATTCAGGTCTTCTTCTTCCGCACC
GGCCAGAACTGGGGCAACCGCGCCTATTTTCCGAAAGCCGACAGTTCGCTCGGGCCAGCGGAAGTGCTGGG
CGCTTTCCTGTCGCAGTTCTATGATGACAAGCCTTGCCCGAAACTCGTTCTCCTTTCGGAAACGGTCGAAG
AACAATCCCTTATCACCGAGGCGCTCTCCACGCGCGCCGGTCATAAGGTCCAGGTCTCGGTGCCGCAGCGC
GGTGAAAAGAAAGAACTCGTGCAGCACGCGCTGACCAATGCGCGCGAAGCACTTGGCCGCAGGCTGGCCGA
AACCTCATCGCAGGCGCGCTTGCTACAGGGGCTTGCCGAAACATTCGGCCTGCCGCGCGCACCGCGCCGCA
TCGAGGTTTACGACAATTCCCACATCATGGGCACCAATGCGGTTGGCGGCATGATCGTCGCCGGGCCGGAA
GGCTTTGTGAAAAACCAGTATCGCAAGTTCAATATCCGTTCGACCGACATTACGCCGGGCGATGATTTCGG
CATGATGCGGGAGGTGATCGAGCGGCGCTTCTCACGTCTGGTAAAGGAACATGGAACCCCTGCCGGCGAGG
TGGAGAACCCGGACGCCTTCCCTGCCTGGCCTGATGTCATTCTCATTGATGGTGGCCAGGGACAGGTGGGC
GCGGTGCGCCAGATACTCGGCGAGATGGGCATCAGCGATCTGGTCACGGCAATCGGCATCGCCAAGGGCGT
TGACCGCGAGGCAGGCCGCGAGCGGTTTTTCATGGAAGGCAAGCAGCCATTTACCCTGCCCCCGCGCGATC
CCGTTCTTTATTTCATCCAACGCCTGCGCGACGAGGCGCATCGCTTTGCCATAGGCACCCACCGCGCACGA
CGCAAGAAGGAAATGGTCAGGAATCCGCTGGATGAAATTGCCGGCATAGGCCCTACGCGCAAGCGCGCCCT
TTTACATCATTTTGGAACGGCAAAAGCGGTTTCGCGCGCCGCTGTCGAAGATCTGATGCAAATCGATGGTA
TTTCGGAAGCCATGGCGCGGGCGATCCACGACCACTTCCGGGACAAA
>BR1488
ATGTTCTTCGCGGTGCTGGGCGCTGCGGTGGGCATTACGTGGACCGTGCAGGTGCTTCAGCGTATCAACTT
TCTGACCACAAGCGGGCAAACCTTCCAGACAATCGTCCAGTTCAGTTCACTGCTGATTCCGTCTGCGATTC
CGCTTGTGATGCCCTTTGCGCTCATCATTGCAATTACCCAGACGCTTTCGACGATGAATCAGGATTCGGAG
CTCGTCGTCATCAACGCTTCCGGCGCGCCGCGCAGCGCGGTAATGCGCCCGATTCTGATCCTGGCTGCAAC
AGTTTCCGTGACTTCGTTTCTGATCGCCAATTATGTCGAGCCCTATGCCCGCATAAATATGCGCGCGATGA
TCGCCAATGCCAGCGCCGACCTGATGCATGTCATCATTCAGGAAGGCACTTTCCGCAAGCTTGCCGACAAT
CTCTATATTCAGGTTGCCGAACGCCGGGCAGACGGCAGCATAGGTGGCCTTTTCATTGCGGATTCGCGCGA
TCCGACACTCGATCTCATTTATTATGCGGCCGACGGCGCCATTGCCTCCACGCCATCGGGCGACATGCTGC
TGATGGAAAATGGCGAAGTGCAGCGCCGCAATGTCTCGGACGGAACCGTTTCGATCATCAAGTTCGATTCC
TATGCCTTCGACCTCAACCAGTTTGCCTCAGCAGGTGATGATTTCGTTATTTTTGCCAAAGACAGGCCGCT
TACCTATCTACTGAATCCCGACCCCAACGACCCGATGCTTCAGACAAGGCCTTTGACTTACAAAGCCGAAT
TGCATCGGCGTTTCACACAGTGGATCTATCCGCTCATTTTCGCGATGATCGCGCTCGCCATTGCCGGCGAT
TCCCGCTCGCATCGCGAAGCTCGCGTTTCAGCCTCTTTTTCCGCCATCAGCACGGCGCTTCTCGTCTATTG
GGCTGGCTATTTCGCCTCCGACCGCGCAGACAAGGACCCAAGCTACATTCTCGTCATGTATCTGGTGCCGC
TGGTCGTCTTTCTGGTTACGGGCTTCATATTGCTGACAGGGCGGCGCCTCGGCCTGCCCGATAAGTGGAGC
GATCGCATTCTCGATTTCCTCGACAATACGAGGCGGCGCCTTGGCGAATTCTACGCCCGCTTTACCGGACG
TCCCCGCAACAATGCCGGAGGCCGGTTA
>BR1447
GTGGCGCGTCCGCCCAATCTTGCGATCTTCGATATCAAGATGCCGCGCATGGACGGTATGGAGCTTCTGCG
CCGCCTGCGCCAGAAGTCCGATCTGCCGGTCATCTTCCTcACTTCCaAGGACGATGAGATCGATGAACTCT
TCGGCCTCAAGATGGGTGCGGACGATTTCATCACCAAGCCTTTTTCGCAGCGTCTTCTGGTCGAGCGTGTC
AAGGCTGTTCTGCGCCGCGTGGCGGCGCGCGACGGCACGGCAAAGCCCGCCGGCCAGCAGGCCAAGTCCCT
TGAGCGCGGACAGCTCGTCATGGATCAGGAGCGCCATACCTGCACCTGGAAGGGTGAGCCGGTTACGCTGA
CTGTTACCGAATTTCTCATCCTTCACTCACTTGCCCAGCGCCCCGGCGTGGTCAAAAGCCGTGACGCCCTG
ATGGATGCGGCCTATGATGAACAGGTCTATGTGGATGATCGCACCATCGACAGCCACATCAAGCGTCTGCG
CAAGAAGTTCAAGGCGGTTGACGACAGCTTCGAGATGATCGAAACGCTCTACGGCGTCGGTTATCGTTTCC
GGGAAGCG
>BR1437
ATGCGGCTGAGATTGTTTTCGGCAAAGAAAAAGGCGCCTGATGGCTCCATTCACGGTGAAGTGCCGGCCGG
TGATGAAAAGCTGGCCGGTAATATGGCGTTTGTCGGCTCGCGCAAGCGCCATGGCAACCGCGCGCGCAACC
GGCTCTGGATGGCCATTGCCTGTTTCGCCGGCATTTATGGCGTGATGGCTGGCAAGCTTGTCTATTTCGGC
ATGATCGGTGGCGTGGATCAAGATGCCGCCGGTCCGTTTGTGCACCAGCTTGCATCACGCCCCGACATTCT
TGACCGCAATGGCGAAATTCTCGCAACCGATATCAAGACCGCATCGCTCTATGCGGAGCCGCGCAAGATCG
TGGACCCGGACGAAACCATCGAAATGCTGTCGACTGTTTTGCCGGATCTCGACTGGGAGGCGACCTATAAG
CGCCTGAAGAGTGGGGCCGGTTTCGTCTGGATCAAGCGCGGGCTGACGCCGAAGCAGCAAAGCCAGATCAT
GGCGCTCGGTGTGCCGGGCATTGGCTTCCGCACGGAAAAACGCCGCTTTTATCCCGGTGGCCCGACCGCTT
CGCATATTCTCGGTCTCGTCAATGTCGATAATCAGGGCATTGCGGGCATGGAGAAATATATCGATAGCCAG
GGGCTGAGCGATCTGCGCTCCGTCGGCCTTGCAACGGGGCAGTCGCTGGAGCCGGTGCATCTTTCCATCGA
TATTCGCGTGCAGCACATCATGCGCGATGTTCTGGTCAAGGCCATGGAGCGCTACCGGGCAATCGCGGCTG
GCGCTGTGGTGCTGAACGTCAAGACGGGCGAAGTGATCGCCATGTCGTCCGTACCGGATTTCGACCCGAAT
AATCCGGTGCACGCGCTGGACAAGGATCGCCTCAACCGCATGTCGGCAGGCACTTATGAAATGGGCTCGAC
CATCAAGAGCTTCACGACGGCCATGGCGCTCGATTCCGGCAAGTTCACCTTGCAGTCGAAGCTCGACGCCT
CGCGTCCGCTGGTGTTCGGCCGCCAGACCATCCGCGACTTCCACGGCAAGGGCCGCTGGCTCACCTTGCCG
GAGGTTTTCATCTTCTCGTCCAATATCGGTTCAGGCCGTGAAGCGGACGCCGTCGGCATCGAAGGGCATCG
CGCCTTCCTCAAGAAGATCGGCCTTCTCGACCGTATGCAGACGGAATTGCCGGAAGTGGCGCGCCCCGTTG
AGCCGCGCGTGTGGAAAAAGGTTCATTCCATGACCATTTCCTTCGGTCACGGCATGATGACCACGCCGCTT
CAGACGGCTGTGGGGGCTGCCGCTTTGATGAATGGCGGCAAGCTGATCGAGCCGACTTTCCTGCCGCGTAC
GGAAGCGCAGGCGGCTCGGGCCAGCAAGCAGGTGATCCATCCACAGGTTTCTGCCGATATGCGCTATCTCT
ACCGGCTGAACTCAACTGCCCCTGGCGGTTCGGGCCGGCGCGCGACGGTTCCGGGCTACCGTGTCGGTGGC
AAGACCGGCACGGCGGAAAAGGTGATCAATGGCCGCTATTCCAAGGATGTGCGCTTCAACGCTTTCCTGGC
GTCATTCCCGATGGATAATCCCACCTATGTCGTGCTTACCATCATTGATGAGCCGAAGCCGGAAGAGGGCA
AGTTCAGCGCCACCGCCGGTCTTAATGCGGCGCCCATGGTGGCTGAAATCATTCGCCGTTCCGCATCATTT
CTTGGGGTCAGTCCCGATTTCCGCAAGGAAGCTTTGCCTGCTTCGGTGGAAAACGTCAGCGGCAGGCCCGA
TTTTCAACAGGAAGTGCCGCCAGCAATGGTTTCAAACGAATACGAT
>BR1434
ATGGCGGCAGACGCGCCACATGATATAGCCAATAAAGAGCGGATAGAGCATTTTCGAGCCAAAAGTGTGAA
ACGGTTTGCGTTGGAAAATGCGACAAAACAAATAGTTAGAGCGGTTCCGGCGATTCTGTTAAAA
>BR1426
ATGAGTATTCTTGGTGGTCATGGAGCATCACAACACGGAACCGCAGGGCGCAAGGTGCGCCTGTTAACGGT
TCTGGACGTTGGGTCGAGCAAGGTTTCCTGTGTCATCGCAAGGCTGCGCCCGCATGAAGGCGGCGCGTTGC
TGCCGGGACGCACCCATCGCATGGAAGTGCTGGGCATCGGCCATCAGCGTTCGCGCGGCGTGAAATCCGGC
GTCATTATCGACCTTGATGCGGCCGAGCAGTCGATCCGCCTTGCGGTAGATGCGGCCGAGCGCATGGCTGG
CCTGACCGTCGATAGCCTCATCGTGAATATTTCGGCTGGCCGTCTCAAAAGCGAAACCTTTACCGCAAGCG
TCAATCTTGGCGGCCATGAAGTCGAACAGACAGATATCCGCCGCGTTCTGGCTGCTGGTGCAAAACAGGCG
CTGGCTGCCGAACGTCACCTCGTTCATTCGCTCCCCGTCGGTTATACGCTCGACGGCGAGCGTGGCATCCG
TGAGCCGCTTGGAATGCTGGGCGACAGCCTTGGCGTGGATATGCATGTGCTGACGGCTGATGCGGCCCCGC
TTCGCAATCTGGAGCTTTGCATCAACCGCTGCCACCTTTCGGTGGAGGCAATCGTTGCAACGCCTTATGCA
AGTGGCCTTGCTGCCCTGGTGGGCGACGAGGCGGAAATGGGTGCGGCCTGTATCGACATGGGCGGCGGCAC
GACGACGATTTCCGTTTTTTCGGAAGGCAAGTTCATTCATGCCGATGCGGTTGCCATCGGCGGCAATCACG
TCACGATGGATGTGGCGCGTGGCTTTTCCACCCGTATGGAAGACGCCGAACGGCTGAAGGTCATGTATGGC
TCCGCCCTGCCAAGTGCTGCCGACGACCGCGATCTGATCTCCGTTCCGCCCATCGGCGATGATGAGCGGGA
TGTACCCAATCAATATCCGCGCTCGGTGCTGACGCGGATTATCCGTGCAAGAGTGGAAGAGACGCTGGAAC
TGGTGCGCGACAGGCTGAACCAGTCGGGCCTCGGACATATCGTCGGCAAGCGCGTGGTTCTGACGGGCGGC
GCAAGCCAGCTTCCCGGTATGCCCGAAGCGGCCAGGCGAATCCTTGCAAGAAATGTACGAATCGGGCGTCC
TCTAGGAATAGCGGGATTGCCCGAGGCGGCTAAGGGGGCAGCTTTCGCGGCGACCGTAGGCCTTCTGATTT
ACCCGCAGGTGGCGGGGATAGAAGAGCGTTCGGTCAAAGCAGCGTCCTCCGGTCTGATGACCGGTACGGGT
GGGCGCATTCAACGAGTCGGCCAATGGCTGAGAGAGAGTTTT
>BR1418
ATGTGCTTGGGAAAGGGGGCTGGCTTAGCGGACAGCGCCTTCAAAAGCGTTCGGCATGGAAGTGTTCTTGA
GGTATTCAAGAGCCTTCTTTGCCGATGCAACCAGCGCAGCGAATGCTTCCGGTTCGTGGATTGCGATATCG
GAAAGAACCTTGCGGTCGATTTCGATGCCAGCCTTGGCCAGACCGTCGATGAAACGGCCATAGGTCAGTCC
CTGCTCGCGGACAGCAGCGTTGATGCGCTGAATCCAGAGGGCGCGGAACGAACGCTTGCGGTTCTTGCGGT
CGCGGTAAGCATACTGCTTGGAACGGTCAACCGCAGCCTTGGCGGTGCGGATGGTGTTCTTGCGACGGCCA
CGGAAACCGGCAGCCTGATCCAGAACCTTCTTATGCTTTGCGTGGGCGGTAACGCCGCGTTTTACGCGTGC
CATGTTATGATCTCCTTCTCAGAC
>BR1394
ATGGCAATTGCACCAAAGGCGGGTTTTGCTCGCACCCTTTTCGCAACCGTTGCACTGGGGGCGATGAGTGT
CGCGGGCACTGTGTCGATGGGAACGCCGCCCGCGCTGGCTGCGCAAGGTCCCGCTTCGGTGGCCGATCTTG
CCGAAGGGCTTCTGGATGCAGTCGTGAATATCTCCACATCGCAGACCGTGAAGGACGATGGCGAGGGGGAC
GGCCCGGTGCCGATGCCGCAGGTGCCGGAAGGTTCGCCCTTCCAGGAATTCTTCAAAGACTTCTTCAATGA
CAAGGATGGCGCGCAGGGCGATGATTCACGCAAGGTGCAGTCGCTGGGTTCGGGTTTCATCATCGATGCCG
AAAAGGGTTACATCGTCACCAACAATCACGTCATCGCCGATGCCGACGAGATTGAGGTGAATTTCAACGAT
GGTTCCAAGCTCAAGGCGGAGCTTGTTGGCAAGGATACGAAGACCGATCTTGCGATCCTGAAAGTCGACCC
TTCCAAGCATAAGCTCAAGGCGGTGCATTTCGGCAATTCGGAAAAGGCGCGCATTGGCGATTGGGTTCTGG
CGATCGGCAATCCGTTCGGCCTTGGCGGTACTGTCACGGCAGGCATCATTTCAGCACGCAAGCGCGATATC
AATTCCGGCCCCTATGACGATTTCATTCAGACGGATGCCGCGATCAATCGCGGCAATTCCGGCGGGCCGCT
GTTTGATATGGACGGCAAGGTGATCGGCATCAATACGGCGATCATTTCGCCTTCTGGCGGGTCGATCGGCA
TTGGTTTTGCTATTCCGGCGGAAATGGCCGCCGGTGTCATCGATCAGTTGAAGGAATTTGGCGAAGTGCGC
CGCGGCTGGCTTGGCGTCCGTCTTCAGCCGGTGACGGAAGACATTGCCCAGAGCCTCGGGCTGAAAGAGAC
CAAGGGCGCGCTGATTGCCGGTCTCATCGAAAATTCCGGTGTCGATAACAAGGCGATAGAGGCGGGCGATG
TGGTGATCCGCTTTGACGGCAAACCGGTGGATACGGCGCGCGATCTTCCGCGCCTCGTGGCGGAAAGCCCG
GTTGGCAAGGAAGTGGAAATCGTCGTTATCCGTCAGGGCGCGGAAAAGACCCTGAAGGTCAAGCTTGGCCG
TCTGGTCGAGGACGACAAGAGCACGGAGCCGGCGGTTGAAGATCAGGTGCCCGCGCCGGATGACGGCGAAC
AGCCGGGAGCCCGGCAGGAGACGCCGGACAAGTCGGACAAGCCGAAGAAGGAACACAAGAGCGATGCTGCT
CCAAGCGTTCTCGGCATGAAGCTCAGCAAGCTCGATGATGATATTCGCGGCGAGTTTGGCATTGCCGAAGA
TGTGGAGGGCGTTGCCATCCTCTATGTTGCACCGGGGACGGCTGCCGGCGAAAAGCGCATCGAAACGGGCG
ATGTCATTGTCGATATCGGTCAGGTGACGGTGAAGACGCCGGAAGATGTGAAAAAGCGTATCGATGCACTG
CGCCGCGAGGGGCGCAAGAATGCGCTTTTGATGCTGGCTTCGCGTTCCGGGGAGTTGCGTTTCGTGACGAT
CCGTATCGAC
>BR1364
ATGGGATGTGGTAGGGTAATCGCGGGGGAGAACAATGATGGCACGCGAGCGCATTTATATCTACGACACGA
CCTCGGTGGATGGGCAGCAGACGTTACCCGCCCGCCCGCAGCCGCGAAGCGAGCGAGGACGGGCAAAGGCA
AAAGCGGGCGTTTGCCTGGGAGGGCAATAATGGCACGCGAGCGCATTTATATTTACGATACGACCCTGCGG
GATGGGCAGCAAACGCCCGGCGTTGACTTCTCTGTGGAGGACAAAAAGTCGATTGTCACGCTGTTGGAAGA
GCTGGGCGTCGATTATGTGGAAGGCGGTTATCCGGGGGCCAACCCGACTGATACGGCTTTCTTCAAGCGTC
GCCAGACCGCTCGCGCCAAGTTCGTGGCTTTCGGGATGACAAAGCGGGCGGGCGTTTCCGTTTCCAACGAT
CCCGGCCTGTCGAGCTTGTTGCAGGCTTCTGCCGATGCGGTCTGCTTTGTCGCCAAGAGCTGGGATTATCA
TGTGCGTCTGGCGCTCGGCTGCACCAATGAGGAAAATCTGGAATCCATCACGGCTTCGGTAACGGCGGCGC
GCATGGCTGGCCGTGAGGCGCTGGTCGATTGCGAACATTTCTTCGATGGATACAAGGCGAACCCGGAATAT
GCGCTGGCCTGCGCGAAGGCTGCCTATTCGGCGGGTGCGCGCTGGGTGGTGCTGTGCGATACCAACGGCGG
CACGCAGCCTCTGGAAGTGGCGGCAATCGTTGAAGCGGTGAAGGCGGTTGTTCCCGGTGAGAGCCTTGGGA
TCCATGCCCATAACGATACCGAACAGGCTGTTGCGGTTTCCCTTGCTGCAATCGATGCGGGGGTGCGTCAT
GTGCAGGGCACCTTGAACGGCATTGGCGAGCGTTGCGGCAATGCCAATCTCGTTTCACTCATCCCGACGCT
GGTTCTCAAACCCTATTGGGCGGAGCGGTTCGAGCTTGGCATCAAGCCTGCATCGCTGAAATCCATCACGC
GCATTTCGCGCGCTTTCGATGATATCCTGAACCGCCCGGCCACGGAGCAGGCACCCTATACGGGCACTTCG
GCCTTTGCCACGAAGGCTGGCATCCACGCTTCGGCGCTGATGAAAGAGCCGCAGACCTACGAGCATATTCC
GCCGGAATCGGTTGGCAATAGCCGCCGTGTCATGGTGTCGGATCAGGGCGGCAAGTCGAATTTCATTGCCG
AGCTGGAGCGCCGGGGTATCCGCGTTGCCAAGGACGATGTTCGGCTCGATACGCTGATTGCGCTGGTCAAG
GAGCGTGAGGCCGAGGGTTATGCCTATGAGGGTGCGGACGCAAGTTTTGAGCTTCTGGCACGCGGTATGCT
CAATCCGCAACCGGAGTTCTTCAAGGTTGAATCGTTCCGCTGCCTCGTGGAGCGCCGGTTTGATGCCAACG
GCATGTTGAAAACCGTGTCCGAGGCGGTGGTCAAGGTCGAGGTGGATGGCGAGATGCATATGTCGGTCGCG
GAGGGCCATGGCCCTGTCAACGCGCTCGATATCGCATTGCGCAAGGATCTGGGCCGCTTTCAGGCCGAGAT
CGAGGATCTCGAACTGGTCGATTTCAAGGTGCGTATTCTCAACGGCGGCACGGCGGCGATCACCCGCGTTC
TGATCGAATCGGGTGATGCCACGGGTGCGCGCTGGCGCACGGTGGGCGTTTCCGACAATATCATCGATGCT
TCCTTTCAGGCTTTGATGGATTCCGTCAATTACAAGCTGATGAAAAACCGGGCCATGGCAGGTCTGGCCGC
TGCCGAA
>BR1351
ATGGCCGCAACATTCGATACGATTTTGAAAGGCGCAACCATCGTCAATCATGACGGTATCGGCCAGCGCGA
CATTGGCATTCGTAACGGACGCATCGCCGCTATCGGGTCGCTTGCAGGGAACTCAGCCGGTGAAGTGATTG
ACTGCACTGGCCTGCATATCCTACCCGGCGTTGTGGATAGCCAGGTTCATTTCCGCGAGCCGGGTCTTGAG
CACAAGGAAGATCTGGAAACCGGGTCACTTGCCGCCGTGCTGGGCGGCGTGACGGCCGTTTTTGAAATGCC
GAACACCAAGCCGCTGACCACTTCGGCCGAAATGCTGGAAGACAAGATTCGCCGTGGCCGCCATCGGATGC
ATTGCGATTTCGCCTTCTGGGTCGGCGGCACGCGCGATAATGCGAAGGACGTGGCCGAACTGGAGCGCCTG
CCGGGCGCGGCGGGCATCAAGGTATTCATGGGTTCGTCCACCGGCGATCTTCTGGTTGAGGACGATGATGG
CGTACGCTCGATCCTGAGGAATACGCGCCGCCGCGCGGCTTTTCATTCCGAAGACGAATTTCGGCTCAAGG
AACGTGAAGGATTGCGCGTTGAGGGTGATCCATCGAGCCATCCGGTATGGCGCGACGAGATTGCCGCGCTT
CAATGCACGCAGCGCCTGGTACGCATCGCCCGCGAGACTGGCGCGCGCATTCATGTGCTGCATATTTCGAC
CGCCGAGGAAATCGACTTCCTGAAAGATTACAAGgACGTCGCCACCTGCGAGGCGACGCCGCATCACCTCA
CCCTGTCGGCGGATGATTACAAGGCGCTTGGCAATCTCATCCAGATGAACCCGCCGGTGCGTGACAAGCGC
CACCGTGAAGGTGTGTGGAAGGGgATCGATCAGGGTATCGTGGATGTGCTGGGTTCAGACCATGCCCCGCA
CACGCTGGAAGAAAAGCAGAAGTCTTATCCGGCTTCCCCATCGGGCATGACCGGCGTGCAGACGCTGGTGC
CGATCATGCTGGACCATGTGAATGCCGGTAAGCTTTCACTGGAGCGTTTTGTCGATCTTTCCAGCCACGGC
CCGAACCGCATTTTCGGCATGGCGCGCAAGGGGCGCATCGCGGTCGGCTACGATGCCGATCTGACGATCGT
GGACATGAAACGCCGCGAAACGATCACGCATGAACAGGCAGGATCGAAAGCCGGCTGGACGCCCTATCATG
GCAAGACCGTCACCGGATGGCCGGTTGGCACTTTCGTGCGCGGCATCAAGGTGATGTGGGAGGCGGAAATC
GTCAATCCTCACAAGGGCGAGCCGGTGGAGTTCCTCGAAGCGCTGCCCCGTCGC
>BR1331
GTGAAGATTCCGGCGGTTGGACTGCAATTCTTTCAGGCTGCGGCAGGCTTGCCCGATAAGGGCGTTAAATT
ATCATTTTATAGAAATGTGCCTAGCAATCGCGCGGGCGCGCGTCTATTGACAGGGTTTGAT
>BR1327
ATGACGACTGCCAACGATATTCTGAAACAGATCAAAGATAACGACGTCAAGTTCGTCGACCTTCGCTTCAC
CGATCCGAAGGGCAAGCTCCATCACGTTACGATGGATATCGGCCTCGTGGACGAAGACATGTTCGTCGACG
GTGTCATGTTCGACGGCTCTTCGATCGCAGGCTGGAAGGCCATCAACGAATCCGACATGGTTTTGATGCCC
GATCCGGAAACGGCTCATATCGACCCGTTCTTCGCCCAGTCCACGCTTGTCATTCTCTGCGATATCCTTGA
GCCGCTTTCCGGCGAAAGCTACAGCCGTGATCCGCGTACGACTGCCAAGAAGGCCGAAGCCTATCTGAAAT
CGCTCGGCATTGGCGATACGGTCTATGTCGGCCCGGAAGCCGAATTCTTCGTCTTCGACGATGTGAAGTTC
AAGGCCGATCCGTTCAATACGGGCTTCAAGCTCGATAGCGCCGAATTGCCATCCAACGACGACACCGATTA
TGAAACCGGCAATCTCGGCCATCGTCCGCGCGTCAAGGGCGGCTATTTCCCCGTTCCGCCAATCGACAGCT
TGCAGGACATGCGTTCCGAAATGCTGACCGTGCTCTCGGAAATGGGTGTGACGGTTGAAAAGCACCACCAT
GAAGTCGCTTCCGCGCAGCACGAACTGGGCACGAAATTCGACACGCTGGTGCGCAACGCCGACAAGATGCA
GATTCAGAAATATGTCGTGCATCAGGTTGCCAATGCCTATGGCAAAACGGCAACCTTCATGCCGAAGCCGG
TCTTTGGCGACAATGGCTCCGGCATGCATGTTCACTTCTCGATCTGGAAAGACGGCAAGCCGACCTTCGCA
GGCAATGAATATGCTGGTCTGTCGGAAAACTGCCTCTACTTCATTGGCGGCGTCATCAAGCACGCAAAAGC
TGTGAATGCCTTCACCAACCCGTCCACCAACTCCTACAAGCGTCTGGTGCCGGGCTATGAAGCGCCTGTTC
TGCTGGCCTATTCCGCACGCAACCGTTCGGCTTCCTGCCGCATTCCATTCGGCTCCTCGCCGAAATCGAAG
CGTCTCGAAGTCCGCTTCCCGGATCCGTCCGCCAACCCTTATCTCTGCTTCTCGGCCCTTCTGATGGCGGG
CCTTGACGGCATCAAGAACAAGATCCATCCGGGCCAGGCCATGGACAAGGATCTCTACGACCTGCCGCCGA
AGGAACTGAAGCAGATTCCGACCGTCTGCGGCTCCCTGCGTGAAGCTCTACAGTCGCTCGACAAGGATCGT
GAATTCCTCAAGGCTGGCGGCGTGTTCTCCGACGACCAGATCGACAGCTTCATCGAGCTGAAAATGGCTGA
AGTGATGCGTTTTGAAATGACGCCGCACCCGGTCGAGTTCGATATGTATkACTCGGTA
>BR1324
ATGTTGCGCTATGTCGGCAACTGCCCGCCCCGCCGCCAGCATCAGCGCGAAACCGTCCTTGATGCCGCCCT
CCATCGTCAGGCGGTCAGCCCGCGCCATTTCTTCCGGCGTAAGCAATTCAAACATAACGCACCTTTCTCCA
GCGGCTTTCTGCCTATGATTAATTACGCGCTGCCGGACTTTATGCAAGTTTGCTTGTTTTTACCGCAAGTT
GCA
>BR1304
TTGACCGGCCATATCAAGATTTTCGGCCAATTCGCCAAAATGAAATTCGGGCTGGTAAGGCGCGATCTGTT
CCAGAAGCCGGTCGATCAGCTCTCCAGCCATGATCCAGGGAAAAGCCGGGATATCATAAATGGGCTTATCT
GGATAAAGGACGGCACATTAGCCGCCCGGACGATCCAGCGCGTCTATCAAATAACATTTGAAGCCGAAAAG
CCCAAGCTGGAAAACGGAAAAAAGCGCAACAGGCCCGGCACCGATAATGGCTATCCCGGTTTGCCTTCCGG
CCTCCATTACGCCCTCCTCAATCGTGCTGCCCAGCCTGCCCGGTCAGTTCTGGCGTTCGGGCACCTGAACG
ACAAGCCCCTCCAGATCGTCGGTAACGCGGATCTGGCAGGAAAGGCGCGAGGTCGGGCGGACTTCATAGGC
GAAATCCAGCATATCCTCTTCCATTGGATCCGGCCCGCCGACCGTATCGGCCCAGTCGTCATCGACATAGA
CATGGCAGGTTGCGCAGGCGCAAGCGCCACCGCATTCGGCATCAATACCGGGAATGCCGTTGCGAATGGCC
GCTTCCATAACGCTGGACCCGTTGTCGGCCTCGACCTCGGTTCGCGTGGCGCCATCGGCGGATACGAATAC
AATCTTCGTCATTTTTATTCCTTGAGACACGTCTTCGACGCGATCTTTGTGCTACGCGCCCCTGGCCCGAT
CCTCAGGCTGATCGCGCGATTGATCCCGAAATGGCAGATACGGCCTTATGAATGGAGTTTCAACCACCGTA
AATGGCGGCTCCCCGACTGCGCCAATATGCCCCCGCCCTGTCAAAATCGGCAAAACGGAACGCCGGAGCCA
ATCCCACCTTGCCGCAGGGGGAAAAAGCGCCATATTATCGGCTCA
>BR1290
ATGTACGGACGCGCCCGTTCCGGAGCCCGTTTCCTAGCTATCGCCATGTTGGCCCCACTGGCTGCCGCCTG
CACGACGACTGTGCCGACATCCACCGCCACGCCGGGCAAGACTGAAGCTCCCGTCAGCGCACCGACCATTT
CGCTGCCGCGTTTTACGCCGACGTCTTATGTCGATCCGGCGCTGGCGCAGCCCTGCATCACTGCCGCAGCC
AACAAATATTTCCTGCCGGAGCGCGTTATCAGCGCCGTCAATTCCCGTCCGGGTGCAGGTGGCGGCATGGA
TGTGGATCTGAAGGTCGATCTGCGCACGGCAGTTTGCCATTTGAATTCAAGCGGCGGTGTGCGTGCTGTTA
TCGATACTTCGCCAAAGAGCGCGGATCAGATTGCAGCCGAAGAAGCTGCGGCCAAGGCCGATGCCGCCGCA
GCCGCCCAGCCGAAAAAGGCCGCGGCAAAGAAAAAGAAG
>BR1282
ATGGTAACCTATATCGAAGCGACAAGCGCACCCGTGAAATATACAGGCCAGATCAGGCTCTACGGCCCCGA
AGCCTTCGAGGCGATGCGCAAGGTCTGCAACCTGACGGCACGTTGCCTTGATGCGCTGAACGATATCGTCA
AACCCGGCGTGACCACCAATGAGATCGACCGTTTCGTATTTGAATTCGGCATGGACCACGATGCGTTTCCG
GCAACGCTGAATTATCGCGGCTACACGAAATCGACCTGCACATCGATCAACCATGTGGTCTGCCACGGCAT
CCCGAACGACAAGCCTCTGCGCGAAGGCGATATCGTCAATATCGACGTGACTTATCTGCTTGACGGCTGGC
ACGGAGATTCCAGCCGCATGTATGCGGTTGGTGAAATCAAGCGCGCTTCCGAACGTCTGCTGGAAGTGACC
TATGAAAGCCTTCTTCGCGGTATCGCCGCCGTCAAGCCGGGTGCGAAGACCGGCGCCATCGGCGCGGCGAT
CCAGACCTATGCCGAAAGCGAACGCTGCTCGGTGGTGCGCGATTTCTGCGGTCATGGCGTCGGCCGGCTTT
TCCATGATGCGCCGAATATCCTGCACTACGGCACCCCCAATGAAGGTGTCGAGATCAAGGAAGGCATGATC
TTCACGATCGAGCCGATGATCAATCTGGGCAAACCGCATGTGAAGGTTCTTGCCGATGGCTGGACGGCGGT
GACGCGGGACCGCTCCTTGACCGCACAATATGAACACACGGTCGGCGTCACCAGGGATGGCTGCGAGATTT
TCACGCTTTCTCCGGCCAATATCTTTGGCCCGCCTTCCATGCGG
>BR1250
TTGCGTTCTCTTGCCCAGCGGATCGCTGACAAGGCTTTTTCCCGCCATTGCGCCGATCCAGTCCAGCCCGG
CACGCAAGCGCACCACCTCACCCACAACCACAATGGCTGGCGGTTGCAGCCCGGCTGCTTCCACCTCGATT
GCAGCCCGGCCCAACGTTGTTTCAAGCACGGTCTGGTCCGGCAGGCTGGCATTGCAGACGAAGGCCACCGG
CTCATCTGGCGAACGTCCAGCCTCGATCAGCCGCCCGGCTATGAGGCCGATATGTTTCATCGCCATATACA
TGACGATGACCGGCGACGATCGGGCAATACCCGCCCAATCGACGCCATCGGGCATGGCGCCGGTTGCGTCA
TGCCCGGTCAGAAACGTTACCGCCTGATTGATATCGCGATGCGTGGCGGCAATTCCCGCATAGGCAAGCCC
GCCAACCCCGGCTGTGACCCCCGGCACGATGCGAAACGGAACATGATACTCAACAAGCGTGAGCGCCTCTT
CCGCCCCGCGCCCGAACACGAAAGGATCGCCACCCTTGAGCCGCAGCACCTTCTTGCCTTGCCGTGCAAGC
TCCACCAGCCGCAGCGAGATGTCGCGTTGCCTGGGCGATGGTTTGCCGCCGCGCTTGCCGGCATATTCCAA
CGTGGCGGTCGCATTCTTCAGCGAAAGACAGGAATCATCGACCAGCGCGTCATAGACGATCACATCCGCCT
GTTGCAAAGCGTTCACCGCATGGAGGGTCAAAAGACCGGGATCGCCCGGACCGGCCCCGACAAGCCAGACA
TGGCCGGGCTCCATCACGGGCAAAGCCACTTTGCTTTTCGCGATCACACAGGCAGCCGCTTCGTCAGCTTT
GCTCATACTCTACAACCACGTTCAATTTAC
>BR1213
ATGATGACTGACATGCGCATTCAGAACAAAGGCCGCGTCAACAAGGCCAAGTCCGTTCGCTTCACCTTCAA
CGGCAAGACCTATTCCGGCTTTGAAGGCGACACGCTGGCCTCCGCCCTTTTGGCCAATGGCGAACATCTGA
CGGGCCGCTCGTTCAAATATCACCGCCCGCGCGGCATTCTTTCCGCCGGTTCCGAAGAGCCGAACGCGCTG
ATGGGCGTCTCGCGCGGGGCGGGCCGGTTCGAGCCGAACACGCGCGCCACCGCGCTGGAGCTTTATGACGG
CCTGAAAGCCGAGAGCCAGAACCACTGGCCGTCGCTGAAGCATGATGTGGGTGCGATCAACGATGCGTTCT
CCATGTTCTTTTCGGCAGGCTTCTACTACAAGACCTTCATGTGGCCGAAGAGCTTCTGGAACAAGGTTTAC
GAGCCTTTCATCCGGGGCGCGGCCGGTCTTGGCAAATCGCCATCCGAACCCGATCCCGACACCTATGCCAG
CCGTTATGCCTATTGCGATGTGCTTGTCGTGGGGGCGGGGCCAGCCGGGCTTGCGGCAGCGCTTGAAGCGG
CGAAAAGCGGCGCGAAGGTTATGCTTTGCGACGAACAGGCGGAACTGGGCGGTTCGCTTCTGTCAGAGCCG
GAGCCGGTCATCAATGGCCGCGCAAGCTGGGACTGGCTGGATGAGACGCTGGCCGCGCTCGCCGCCATGCC
CAATGTCACGCTTTTGCCGCGCACCACGGCGATTGGCTATTATCACCAGAACATGCTGGGCCTTTGCCAGC
GCCTGACCGATCACCTGCCGAACCCGCCAGCCAACGCGCCGCGTGAACGCATGTGGCGTGTGCGCGCAAAG
CAGGTCGTGCTGGCGCAGGGTGCAATTGAAAGGCCGCTGGTCTTTGCCGGCAACGACCGTCCGGGCGTGAT
GCTGGCGGGGGCCGGGCGTACCTATCTCAACCGCTATGGCGTGAAGGTTGGCCACAAGGCCGTTATTGTCA
CGTCGCATGATAGCGCCTGGCTTGCCGCCTTCGATCTGGCGGTCGCAGGCGTCAAGGTTCCTGCCATCATC
GATGTGCGTGAGCATGTTGCGGGCAGCCTCGTCAATCGCGCGAAGATGCTGGGCATAGAAACGCTGACCGG
CTGGACCGTAACCGATACGGGCGGGCGTCACCGGGTTTCCTCCGTGCGCGCCAATCCGGTGCAGGGCGGCG
TGGCTGGCGCGCCGCGCACCATAGAATGCGATGTGGTGCTGATGTGCGGCGGCTGGACGCCAAGCGTCCAT
CTCTTTTCGCACACCAAGGGCCAGCTTGTCTGGGATGAGGAGCGCCAGATCTACCTACCGGGCGCACGCAC
GGAAGAAAGCCGCTGCGCTGGTGCGGGCAATGGTCATTTCGACCTTGAAGCGGCGCTTCGTGAAGGAGCGC
AAAGCGGTGCCGGTGTGGCATCCGATGCCGGTTACAAGGCAAGCGCGCGCGAATATGCGGTGGCCGGTGAT
TTCATCTGCAATGGCATAAGCTGCCGCGAATTGCCGACGGATCGTGATCCGGGCAAGGCCAAGGCCTTCAT
CGACTTCCAGAACGATGTCACCGCCAAGGATATCCGCCTTGCCGTGCGCGAAGGCTTCCGCTCCATCGAGC
ATGTCAAGCGCTACACCACAAACGGCATGGCGACCGATCAGGGCAAGACGTCCAACATCAACGGTCTGGCG
GTTGCGTCCGATGCCTTGAAGCGCCCTGCGCCGCAGGTAGGCCTCACCACGTTCCGCCCGCCCTATACGCC
GACCACTTTTGGCGCTTTCTGCGGCTATAATCGCGGAAAACTGTTTGAAGTCACGCGCAAGACGCCGATCG
ACGCTTGGGCCGAACAGCATGGCGCGGCTTTCGAGCCGGTGTCGCTGTGGCGGCGCGCCTGGTATTTCCCG
AAGCCCGGCGAAGACATGCATCAGGCCGTGGCGCGTGAATGCAGGGCAACCCGCCAGTCGCTCGGCATGTT
CGATGCCTCCACATTGGGCAAGATCGAGGTGGTCGGCCCGGATGCAGCCGAATTCATGAACCGCATGTATA
CCAATCCATGGACGAAGCTTGGTGTCGGGCGCTGCCGCTATGGCCTGCTGCTCGGCGAAGATGGCTTCATC
CGCGATGACGGTGTGGTCGGTCGCCTGACGCAGGACCGTTTCCACGTGACGACCACGACCGGCGGTGCGGC
ACGCGTCCTCAACATGATGGAAGACTATCTCCAGACTGAATGGCCGCAGTTGAAAGTTGCGCTCACATCCA
CCACCGAACAATGGGCGGTGGTGGCGATCAATGGCCCGAACGCGCGCAAGCTTATCGAGCCGATGGTCGAG
GGGCTGGACATTTCGGACGAGGCTTTCCCGCATATGTCGGTTGCCGAATGCACATTCCTCGGCGTGCCTGC
GCGTCTCTTCCGCATGAGCTTCACCGGCGAACTCGGCTTCGAGATCAACGTGCCGTCGCGCTATGGTCTTG
CGCTGTGGAAGGCGCTTTACGAGGCGGGCCAGCAATATGACATCACACCTTACGGCACCGAGACGATGCAT
ATTCTGCGCGCCGAAAAGGGCTATATCATTGTCGGCCAGGATACGGATGGCACGGTGACGCCGGACGATGC
CAGCCTTGGCTGGGCCATTGGCAAGCAGAAGCCGGATTTCGTCGGCAAGCGGTCGCTGTCGCGTCCCGACA
TGCTGAAAAAGGACCGCAAGCATCTGGTCGGGCTTCTGACCAAGGACCCGAAGCTGGTGCTGGAGGAAGGC
GCGCAGATCGTGGCCGATCCAAAACAGGCGGTGCCGATGACCATGCTTGGCCACGTCACGTCTTCCTATTG
GAGCGAGACGCTTGGCCGTTCCATTGCCATGGCGCTGGTGTCCGGTGGCAAGGACCGTATGGGCGAGACAA
TATATATGCCGATGCCGGATGGCAGTGTGCATGAGGCCATCATTTCGGGAACCGTCTTCTACGATCCCGAA
GGTAAGAAGTTGAATGCA
>BR1207
ATGAGGGAGCCGACCTTGGAAACTGACGATCGCTATCGCGCGCCCGCGCTGGACAAGGGGCTGGACATATT
GGAATTGCTGGCGAGTGTGGATGGCGGGCTGACACAGGCCGAGATCGCCAAGCATCTCGACCGCAGCCCCA
ACGAATTTTACCGGATGCTGGACCGGCTGGTGAAGCGCGGCTATGTCACCAAACTCGATGGCGACCGTTAT
TCACTGACGCTGAAGCTGTTCGGGCTTGCGCAACTCCATGCGCCGGTGCGCAGGCTTGCCTCCTTCGCCAC
GCCCTTCATGCGCGAGCTTGCCGACCGTTCCAAACAGGCCAATCAGCTTGCCGTGTTCGACCGCGGCTCGG
TGGTGGTCATCGCTCAGCAGGAAGCACCGGATTACTGGGGCATTTCCATCCGCGTCGGCTCGCATATCAGC
CTGTTCGATACCGGCTCCGGTCACGTTCTGCTCGCCTTCCGCTCACCGGAAGAGCGCGAAATGATGATTGC
CGCCCATGTGAAAAGCCGTGATGAAGTCAATCTGGATCAGGACTTCTATGACCGTCTCGACCAGATACGCG
AGCGCGGATACGAGATGATGGCGAGCGCACAGATGGCGGGCGCCTATAATCTTTCCGCGCCGATCCTCGGC
CCGGACGGCACCTGCATTGCAGCCCTCACCTGCCCCTATATCACGCTCGTCAATCCGTCTTCGGCCCCTGA
TATCACCCAGACGATCAGCCTGTTGCAGAAGACGGTACGCGACCTCTCCAAGCTGGTGGGCGCGGATGTAG
GCGTGACAGGA
>BR1205
TTGCCATGGCGTGCAGCCATCCACGCGGGCAGCTTCCACCGGGTCCTGCGGCATGGCGAGAAGCCCGGCCA
GAATGAGGATTGCAAAGACGGCGGTGGACGACCAGACTTCGGCGGTCAGGATCGCCACCAGCGCCAGATTG
CCATCGATAAGCCAGGGGATCGCCCTGTCGGTCAGCCCCAGCGACTGAAGCGCATTATTCACCAGGCCGAT
ATTGTCGTTGAACATGAACTTGAACTGGAAGCCCACCAGAACCGGCGAGAACATCATTGGAAACATTATGA
GCGTGCGCAGGGTGCGCTTGCCCCATGTGGCCTTGTTGACCAGAAGCGCCAATCCCAGCCCCAGAAGCATT
TCCAGATTGAGTGCGATGGTCAGAAAAACCACCGTGCGCAGAAAGGCTGCCCAGAACACCCAGTCACTCAG
GATGCGCTCATAATTGCGCAGGCCGATAAAGCGGAACAGCGTTTCCGGGCGCGTCAGGCGAAACGGCGTGA
AGCTGGAATAAAGCGAAAGCGCAAGAGGAAAGAGCACCACCGCAGCCAGAATGATGAAGGCCGGAAGCAAC
AACAGGACCGGCGGGGAAAGTCTTTTAAACATTATCCATCCCAAATACTGGGTTCGGCGCATGAGCTGCCG
AAACGGCCCGCCACCCCTTGCGGAAAAGCGGGCCTGCCGAAAATCGGACGGTCAAAGGTCGCCGGCGTCTT
CCAGAATCTGGGTCGCCTTCTGGGCCGCACTGTCGAGTGCTTCCTTCGAGGTCTTGTCGCCCAGAATGGCA
GCCTGCAATTCAGGATAGACGGCGTTGGAAATCTCGATCCAGGACGGGGTGCTCGGAACCGGGAAAGCATT
CTTCGCCGCTTCCTGGAACACGGTCAGCACTTCCTTCTTGTAAGGATCAGATGCAGCCTGCTCGATATCCC
ATTCCCAGACCTTGGTGCGGGTGGGCAGCGTACCATTGGCGGCTTCCACCTTCTGGGAATCCTCATTGGTC
AGGAACCAGACCAGCGAAGCGGCAGCCTCCTTGGTGGGGCAGGCTTCGGTGACGGAGAAACCGTGATGGCC
AGACCAGCCGGTGCGCTTGCCGGACGAGCCCTTGGGCTGCACGACGATGCCGACATTGCCCGCCACCTTCG
ACGACTTCGGATCGTTGAAATAGGTGGCCCAGCCCGGCCAGTCCAGATCGAGCGCCACGGTGCCGGAAGCA
AAGCCCGCGCCAAGATCATCCCAGAGATAATTCGTGGTGCCAGCCGGGACGGCCTTGGCCTTGTAGAGGTT
TACGAACCAGTCGAGCGCGCGCACGCCTGCATCGGAATTGAAGGCAGGCTTGCCGTTCTTGTCGAGATATT
CACCACCCTCGGCAACCAGCATTTCATAGAAGCGGCCATTGATCGCCTCTTCCTTGCCTGCGAACTGGGTG
CCGTAGAAATTCGGCGGATCGGCGAAGAAGATCGCCTGATCGGAAACTTCCTTCCAGGTCTTTGGCGGTGC
CAGATCGTAGCCGTATTTTTCCTTGAACGCCTTCTTCCTGGCTTCGTCCTGATAGAGGCTCTTCTGGTAAT
AAAGGGCGGACACATCGAACTGGGCACGCGGCAGCATGACCAGCTTGCCATTGAGCATGGACGCCTTGATG
ACCGACGGCACGAAAGCGTCGATCTCTTCCTTTGGCAAGAGCGGTTTCAGGTCCGTGTAAAGATCGCCATA
TTGCGGCGCGAAAGAAGAATGGTTGGAACCGACGCACCAGTTGATCGAGCCGGAAGCCATGTCGGACTTGA
TTTCCTTGTCGAGCTCAAAATGATTCTTCTTGGAAAGAATGTTTACCTTTGCGCCCGTCGCCTTCTCCCAA
TCGGCAATGCGCGCATAAAGCGTTTCATATTGCTGACCGCCGATCAGCTTGGCGTTAACGGTCACGCCCTC
GAACTTGCCGGGCAGATCCGCCGCGTAAGCCGGGGCAATTCCCATTGCCAGCAAAGCCGCGCCCGCCAGAA
CATGCGATTTGAAATCCTGCATTCGTCTCCTCCTTAT
>BR1204
GTGGTAGTCATGGCCCAATCCTTGAGCAATGCCCAACACCGTATGCGCCGCCGCATTGCGCGCGTTTTCTA
TCTCGTGGGCCTGTTTCTGGCTATGCTTGTCATATGCCTGCCGGGCTTCTGGATCGTGCTTTCCTCCTTGC
GTCCGCCGGTGGAAATCATGGCCAAGCCGCCTGTCTGGATTCCGCAGGACATCTCGCTCGATGCCTATCGC
ACCATGTTCTCAGGAGCAGGGGCGGGCGGCGTGCCGGTCTGGGATTATTTCCGCAATTCCATCATTGTTTC
CGTCACCTCCACCGTGGTGTCGCTCATCATCGGCATGTCGGGCGGCTATGCCTTTGCCCGGTTCCGCTTTC
GCGGCAAGTCGGCAACCTTCCTGGGCTTCATGCTCACACGCTCCGTGCCGGGCATCGCCCTGTCGTTGCCG
CTCTTCATGGTCTATTCGCGCATTGGCATCATCGATACGCATTTCGGCCTGATCCTTACCTATGTGGCGCT
GAATATTCCCTTCACGATCTGGCTGATCGACGGCTTCTTCCGGCAGGTGCCGAAAGATCTGGCCGAAGCCG
CCCAGATCGACGGCTGCACGCGCTGGCAGGCCTTCTGGCAGGTGGAATTTCCGCTTGCCGGTCCCGGCATC
GCATCGGCGGGCATCTTTGCCTTCCTCACCTGCTGGAACGAATATGCGCTGGCCTCCCAGCTCACCCGTTC
CGTCAATTCCAAGACGCTTCCGGTCGGTCTTCTTGATTATACGGCCGAGTTCACCATCGATTGGCGCGGCA
TGTGCGCGCTTGCCGTGGTGATGATTGTTCCGGCGCTGGCGCTCACCTTCGTCATCCAGAAACACCTCGTT
TCCGGCCTGACTTTCGGCGCGGTTAAAGGT
>BR1185
ATGACGAAGCGAGTGCCCCTTACGAAAGAATTTGAACGTGAGGCTGTACGACTTGCGGAAACGAGCGGTCG
TACGCAGAAGGAGATTGCTGCAGATCTTGGTATCGGCCTGTCGACGCTGACACGGTGGATTTCCCGCAACC
GGGATGCAACCATTGATGATCCCGAACTGATTGATGCAAACGAAGATATGGTGGCTGAACTACGGCGTCGA
CTCATAAACTTTGGCACCATATTCTTGTTGCGACGAGCTTTACGGCTGCGAGGTAGTTTTCCGGGCGCTTA
TCATATCGGGTTGCGATGCCTCGGAACTGCT
>BR1157
ATGTCAAATCTGCAAACCCGCATCATCACCGCTATCGTTCTGGGCACCATCACCTTGTGGCTGACATGGGT
TGGCGGTGTGGGCTTTACGCTCTTTTcCATCGCGATTGGCCTTGCCATGTTTTACGAGTGGACGGAACTGT
CCGCGACACGGCAAACCGCCTTTTCGCGCCTTTTCGGCTGGGCGTGGCTGATCGTTACCGGGATTCTCCTG
ATCCTTGACCGCGGTGCATTGCTGACAATCGGGTTTCTTGTCGCCGGTTGCGCCATTTTGCTTGTCACACA
GTGGAAAAGCGGTCGTGGCTGGCCTGCTGCCGGGCTTTTCTATGCGGGTTTTTCGGCGCTTTCACTTTCGC
TCTTGCGGGGGGATGAGCCGTTCGGTTTTACCACTATCGTCTTTCTCTTCGCCGTCGTGTGGTCTACGGAT
ATTGCGGCCTATTTCAACGGGCGGGCCTTGGGCGGGCCTAAGCTTGCGCCACGTTTCTCGCCCAACAAAAC
GTGGTCAGGCGCCATCGGCGGGGCTGCGGCTGCGGTTGCGGGCGGGCTTCTTGTCGCCTCGCTTGTGGCGG
CTCCGGGAGGCTGGGGCGTGCCGGTGCTGGCGCTGCTCCTGTCCATCGTTTCCCAGATTGGCGATCTTGCC
GAATCGTGGGTGAAACGCCAGTTCGGTGCAAAGGATTCCGGCCGTCTTCTTCCCGGCCATGGCGGCGTACT
CGATCGCGTTGACGGGCTTGTCGCCGCTGCCGCACTTTTGTATCTCTTTGGTGCGATTTTTGCCGAGCCAG
ATGTTCCGTCTGCAATCTTCTTTAGTTTC
>BR1156
TTGCAGGAGGCTCTGGCCCTGTTTTTCGGTAGCGAAAGTCTGTTGGTCGGGACAATCATACCGTTTCTGTT
CGTTTTGACCGTCGTGGTGTTCGTTCATGAGATGGGCCACTATCTCGTTGCGCGGTGGTGCGGCATCGGAG
CCCAGGCTTTTTCCATCGGCTTCGGGCCGGAACTGCTGGGCTTCACCGACCGCCATGGAACGCGGTGGAAA
CTTTCGGCCATCCCCCTCGGCGGTTATGTGAAGTTTATCGGCGATGAGAGCGAAACCAGCTCGCCTGTAGG
CGTCAACGAGAGTGCTCTCAGCGAAGAGGACCGCAAGCGGGCCTTTCATACCCAACCCGTCTGGAAACGCG
CGGCGACGGTTTTTGCAGGTCCGGCGTTCAATATCATTCTTACCATTGCAATCTTCAGTGTTTTCTTTGCG
CTTTATGGGCGCCAGATCGCCGATCCGCTGATTGCGGGCGTACAGCCGGGAAGCCCGGCAGCCGAAGCCGG
GTTTGAACCGGGGGACCGCTTCGTCAGCGTGGAGGGCGAGAAGATAACGACTTTCGCCGATGTGCAGCGCA
TCGTTTCAGGCCGTGCCGGCGACAAGCTGAATTTTACCGTCGAGCGTGACGGCAAGATGGTGGACCTTCAG
GCAGTGCCGAAGATTGTCGAGCGGACCGATCCGCTTGGCAACAAGGTGAAGCTTGGCGCTATCGGCGTTGA
AACCACGGAAGCTGTGGGCAATTTCCGGCGTATTGAATATGGCCCGCTCGAATCGGTGGGGCAGGCCGTCA
TTGAAACCGGCCATATTATCGGTCGGACCGGCGAGTTCTTCAAACGTTTCGCCGTGGGGCGCGAAGACAAG
TGCCAGCTTGGCGGACCTGTGAAGATTGCCACTATGGCCAGCAAGGCGGCAAGCCAGGGGTTTGACTGGCT
GATACAATTGATGGCCATGCTATCCATCGGTATCGGGCTGTTGAACCTGTTTCCGCTCCCGCCGCTCGATG
GGGGACATCTGGTTTTCTATGCGGTGGAGGCAATCAAGGGCAGCCCTGTTTCAGGTGCGGCGCAGGAAATT
TTTTATCGCATCGGTTTTCTCCTTGTCATGGGATTTATGGGGTTCGTACTTTTCAACGACCTTTTTGCCTG
C
>BR1154
GTGGAGGATGGCAGCATGACGAGGCAGATTTCGGCAGCGGAAACGCGCATTATCCGTTTTTTGAAGCAGGG
CGGTTGCGAGCTTCAGGATTCGGTTCGCGAAACGCACGCTTTGCTGGTTGGCGAACAGGGGACTATTGCGG
CTTCGCGCCTTGAAATCGAACGGATGCGCAAGGCTGGAACCATACAATGTGACGGGCAGCGCATCGCGCTT
TCTGCAATGGGACGCAAGCGTCCTAAAGAGCAGAAAAAGCCCACGCTAACCGCCGCCTCTGTCCGTGAGAC
ATACGAGATAGAGCCGGGCGAAAAGGTGGAGATCAATCCGGCGGAATCCCCACTTGCCATGCTTTATCGGC
GCCGGAACGCCGATGGCCGCGCCTTTATTTCGGAGGATGAATTCCGTGCGGGCGAGCGCCTGCGCGCTGAT
TTCACGCGGGGGTCGCTCATGCCTTCCGTTACGTCGCGCTGGGATGTCCAGGCCGGGTCGGGCGGGCATTA
TGGGGCAGGCGGCATGGCTGAACTGACCGATATCGCCCTTGCAAGCCGGATAAGGGTGGAGCGTGCGCTGG
AAGCTGTCGGGCCGGAACTGAGCGGCATTTTGATAGACGTTTGCTGCTTTTTGAAAGGACTGGAAACAGTC
GAGCGTGAGCGTCAATGGCCGGTTCGTTCTGCCAAGGTGCTGCTGAAAGCGGGGCTTGCCATGCTGCATCG
CCATTATAATCCTCCGCGCGAAGGCGAAAGGCGAAGGGGGGCAGTCCTGCATTGGGGGGCGGAGGATTATC
GCCCTGAGATTCGACCGCGGAGTGTG
>BR1137
ATGACGATGTTTCTTGTGACGCCACCGGCGCTCGAGCCGGTGACGATTGCCGACGCACGCGCGTTCTTGCG
AATTTCTACCGAGAGTGAGGATGATATCCTTGGCCGCCTCATCACCACCGCGCGCGAGTTGGTGGAAGCCG
AAACGGGGCTGGCGCTCATTGACCAGACCTGGCGTCTGCGCGTTGATCGCTGGCCGCGTTCGGGCCGTCTG
GCGCTGTTCAAATATCCGATCAAAGCGGTGACGGGCGTTGTTGCCTATCGGCCGGATGGGGCGGCGATCAG
CTTTTTGCCGGACGAATTTGCGCTGCATCATGACAGGCGCCCGCAGCGTCTTTACATGGCGCAATATCCCG
ATGCCTCCACATTCTGCGGTATCGAAGTGGATTTCATTGCAGGCTTTGGTGAGAGCGGCGTTGAAGTGCCT
GATACACTGAAACAGGCAATCCTTACCCTGACCGCCCATCTTTACGAATCGCGCGCAGGTGTGAACGCCGA
TGCCGTTCAGCGATCTTTCCCGCCCATCGTGAACCAGATGGTCGATAGCTGGCGGCGGATTTCCCTA
>BR1116
ATGCGTTGCATAGATGCAACGCTATTGGTTATTATGGTTAACATGAACAGGGCGAATCTGACGACCGACAT
GAATGGGACGAGGCAATCATCGCAAGATGAGGGGCAGGGTGAGGGGCGCAGGCTTCTCGCCTTACCGGGTA
TTATTACCGTCGTTTCGGCGCTGGTCACAGCTTCGTTTTCCTTCGCGATCCTGATCGGTATTACGCCAATC
ACCCCCGACCGCACGGTTACACTGGCGCTGGTGATTATCAATGTCGCGTTGATTTTGTTTCTGATCCTGCT
GATCTGCCGCGAGGTCTATCGCATCATAACGGCGCGCAGGCTGGGCAAGGCCGCGTCGCGCCTGCATGTGC
GCATTGTAGCCCTGTTTTCGCTGATTGCCGCCATTCCGGCAATCGTGGTGGCAATTGTCGCTTCCGTGACC
CTCAATCTGGGCCTTGATCGCTGGTTCGACCAGAATACACGCGACATCATCAGTTCATCCCAGAATTTCTA
TACAGCCTATCTTCAGGAAACCGCGTTGAACCTCCAGAGCACGTCCTATTCGATGTTGCAGGATCTGGATG
CGCAGCGTGCCATTTATAGCCTCGACCGGAACGGTTTCATCCAGTTGATGACATTGCAGGTGCGTGGGCGC
GCCTTGCTGGGCGCATTTCTGGTGCGTGATAATGGCGATGTCGTCGTGCAGAGCGATACGGGCATGGAAAG
CCATCTGCCGCCGCCGACGAAGGATTCGCTTGAAAAGGCACCGGACGGAAAGCCTGTCATCATCCCGCCGG
GAAACACCAATTTCGTCGGTGCACTCATCAAGATGCGCGAAATTCCCGACATGTATCTTTATACGGTGCGT
TCGGTTCCGCCGCAAATTCTTGATGCCATCAATCTGATGAATGCCAATGCCAGGCGCTATCAGGAAATGGA
TGCGAACCGCATTCCGACGCAGATTGCTTTCGCTTTGCTCTATTTCGGCCTGACGCTGATCGTGCTTCTGT
CGGCCATCTGGACGGGCATTGCCGTTGCAGACCGGCTGGTGCGGCCAATCCGGCTGCTGATCGGCGCATCG
GACGATGTGGCTGCCGGCAATCTTGATGTTTCGGTGCCGGTGCGCTCGTCCGATGGCGATGTGGGGGCGCT
GTCGGGTACTTTCAACAACATGGTTGCCGAGCTGAAAAGCCAGCGCAACGAACTTATTTCCGCCAAGGACC
AGATCGATGAGCGCCGCCGCTTCTCCGAAGCCGTGCTTTCGGGTGTGACGGCAGGGGTTATCGGTATTGAG
AATGACGGGTCGATTTCGATCCTCAACCGCTCTGCGGAGCATATGTTTGGCGTAACGTCGGAAGATGCGGT
CGGGAAAAGCCTTACCAGCATCGCGCCGGAGGTCGGGCAGGCTTTCGAGGTGGCGCGCGCCACGGGGCGAA
CCGTGCACCGCGAGCAGGTGAGCATGGCGCGCGGCGGCGGTAGCCGCAGCTTCAATGTGCAGGTAACGGTC
GAGGATGCCGAATCCGAGGATCACTCCTATGTGGTGACGGTGGACGACATCACCGATCTGGTGCAGGCGCA
ACGTTCTTCCGCATGGGCGGATGTGGCGCGGCGCATCGCGCATGAAATCAAGAATCCGCTGACGCCGATCC
AGCTTTCGGCAGAGCGCATCCGCCGCCGGTATGGCAAGGTCATCACGGAGGATCGCGAGGTTTTCGACCAG
TGCACGGACACCATCATTCGTCAGGTCGGCGATATTGGCCGCATGGTCGATGAGTTCTCCGCTTTTGCGCG
TATGCCCAAGCCGGAAATGCGGCATATGGACCTGCGCGAAGCATTGCGTGAAGCCTCGTTCCTGATTGAAG
TCAGCCGCAGCGACATTCATTTCGACAATGATTTCGGTTCGGAAAAGCTGATTGGATCGTTCGATAGCCGC
CTGATCGGGCAGGCCTTCGGCAATGTCATCAAGAATGCCAGCGAGGCCATTGACGCGGTTGCGAAGGAAGA
GCGCGGCGAGGGGCATATCCGCATTCACGCCTACAGGGCGGATGGGCAACTGGTGGTCGATGTGATCGACA
ACGGCAAGGGCTTGCCCGGCGATGATCGCCAGAAACTGCTGGAACCCTATATGACGACCCGCGAAAAGGGA
ACGGGCCTTGGCCTCGCAATCGTTCGCAAGATTGTTGAAGATCATGGCGGTAATCTTGAATTGCATGATGC
GCCCGCGGATTTTCACGCAGGCCGGGGCGCAATGATACGTATGGTCTTTCCCGAGGATCCCCCCATTCCAT
CCGATGCGGATTCGGGAAATGACGAAAAGCACACAGCTGGACAGGTGATT
>BR1114
ATGTCCAGAGCCAGGATTTCCAACTATCGCAAAGGCGTTGCCGCCGTCGCGCTTTCCGCAGCGCTTGCCGG
CGCATTCGTCGTAACCGGCCCGCTTGGTGCGCTGAACGAAGCCCGCGCCGAAGCGGTGCATGTGACGCCGC
CGCCGCAGGCTGGCTTTGCCGATCTGGTGGAAAAGGTTCGCCCTGCGGTCGTCAGCGTCCGCGTCAAGAAG
GACGTGCAGGAGACTTCCAACCGTGGTCCGCAGTTCTTCGGCCCTCCCGGCTTCGACCAGTTGCCGGACGG
TCATCCGCTTAAGCGATTTTTCCGTGATTTTGGTATGGAGCCGCGTGGCGATTCACGCTCCGATAACCGTC
GCGGCAAGGCCAACAAGCCGCGCCCGGGCCATGAACGCCCTGTTGCACAAGGATCGGGCTTCGTCATTTCC
GAAGATGGCTATGTCGTTACCAACAACCATGTCGTTTCCGATGGCGATGCCTATACCGTCGTGCTGGACGA
CGGCACCGAACTTGATGCCAAGCTGATTGGTGCTGATCCGCGCACGGACCTGGCCGTGTTGAAAATCAACG
CTCCGAAGCGCAAGTTCGTCTATGTCGCCTTTGGCGACGATAATAAGGTGCGCGTCGGTGATTGGGTTGTC
GCAGTTGGCAATCCGTTCGGCCTTGGTGGCACGGTGACTTCCGGTATCGTTTCGGCCCGTGGCCGAGACAT
CGGCGCAGGCCCCTATGACGATTTCATCCAGATTGATGCCGCCGTGAACAAGGGCAATTCCGGTGGTCCGG
CTTTCGACCTTTCCGGCGAAGTCATCGGCATCAACACCGCCATCTTCTCGCCATCTGGCGGCAGCGTGGGC
ATTGCTTTTGCAATTCCGTCCTCCACCGCCAAGCAGGTCGTGGACCAGCTCATCAAGAAGGGTTCCGTCGA
GCGCGGCTGGATCGGCGTCCAGATACAGCCTGTGACCAAGGATATCGCCGCTTCGCTCGGTCTTGCCGAGG
AAAAGGGTGCGATCGTGGCCTCGCCGCAGGATGATGGCCCGGCTGCCAAGGCTGGCATCAAGGCGGGCGAT
GTGATTACGGCTGTCAATGGCGAAACGGTTCAGGACCCGCGTGACCTGGCGCGCAAGGTTGCCAATATCGC
TCCTGGTGAAAAGGCGGCCCTGACCGTCTGGCGCAAGAACAAGGCCGAGGAAATCAACGTCACGATTGCTG
CCATGCCCAATGACAAGGGGAAGAGCGGCAGCCAGTCCAACGACAATGACGGCGGACAGGGTGAAACGCTC
GACTCCTACGGCTTGACCGTCGTTCCGTCTGAAGACGGTAAGGGCGTGGTCGTTACCGATGTGGACCCGGA
CAGCGATGCGGCTGACCGTGGCATCCGCTCCGGCGATGTGATCGTGAGCGTCAACAACCAGACGGTAAAGA
CCGCTGGCGATATCAACAAGGCCATCACGGCAGCCGAAAAGTCAGGCCGTAAGGCGGTGCTGCTGCAATTG
CAGAGCAATGATCAGAGCCGCTTCGTGGCGCTGCCGATCAATCAGGAA
>BR1089
ATGCGCCCGCGACGGCGTGATAACGAAGGGAAGACACGTTTGAAAGAGCTAATTGCCGACTCCGATGCCGC
AGGCAAGCGTCTGGACCAGTGGCTGGCCGCTGCATTGGGGCCGGAACTCTCCCGCAGCCGCGTTCAGTCCC
TGATTGCCGATGGCCATGTGACCATCGACGGCGCGCCCGCCCGCGAGACGAAACAGAAACTGCGCGAAGGC
ATGAACATCGCCATCGTGCTGCCTGAGCCTGAAcCCGCCGAACCGCAAGGCGAGAACATCCCGCTCGATAT
TCTTTACGAGGACGATGATCTCATCGTGATCAACAAGCCTGCCGGGCTTGTCGTTCACCCAGGCAATGGCA
ACTGGACTGGAACGCTGGTCAATGCCCTCATNNNNNTTAAT
>BR1062
TTGCCGGACCGATTATGCCGGACAAATCATTCCAAATTCATGAATTTTCGCGCGCCAAGCGGCCTCAGCGG
CCCCTATTATGCGTCCGCTCATATAGCTGGCGAACGA
>BR1060
GTGGCCGAAACCAATACCTCGCCTTCTCCCGCTCAAGACCAGTCTCCCAAACATCCAATGCTACGTCGGAT
TCTGCTTATCGTAGGGCCGGTCGTCGCGATTGCCATCGGCCTGTGGATCTATTTCAGTGGAGGGCAATATG
TGAGCGAGGATGATTCCTATGTCGGCGCGGCGGCAGTGGCCATAACACCGCAGGTTTCCGGTCAGGTTATA
CGTGTCGCCGTTGGCCCGAACCAGCCCGTCAAGGAAGGCGATCTTCTGTTTGAAATAGACCCACAGCCGTT
CCAGATCGAACTTGACCAGGCAAAGGCCAATCTGGCGCAAGCGAGCGAAAAACTCGCAGGGCTTGTCCTCA
CCTACAAGCAACAATTGGCAAGTGTGGCTCAGGCAAAGGACGATGTCACCTTCGCACAGGAGCAATTCAAC
CGTGTTTCGGCACTGGTTCACGACAAGTTTGAGGCGCGCGCGCAATTCGATCAGGCTGAGCGGGCCTTGCA
GGTGGCCAAGCAGGAGCAAAATGCCGCCGCGTCGGCAGCCGCCGCAACACTGGCACAGCTTGGCGGCACAC
TCGATAAACCGTTACAACAACATGCATCCTATCTGGCGGCCAAAGCCGAAGTGGAGCTTGCCGAACGCAAT
GTCAGGCTAACCCGCGTGCTCGCCCCATTCGCCGGAACAGTGACGCAAGTGGAAAACATCCAGCTGGGAAG
TTTTCTTGCCGTAGGCGATCCGGCTTTCACCCTGATCGGCGCCGATGCCTGGGTAAACAGCAATATCAAGG
AAACGGACCTGACCCATATCAAGGTTGGCGATCCTGCCACGGTCGTGCTCGATTCCTATCCGGACCAGCCC
CTGCAAGGCGAGGTGCAGAGCATAACGCCCGCAAGCGGTTCTGTTTTTGCCCTGCTGCCCGCCCAGAATGC
CTCCGGCAACTGGGTCAAGGTGGTGCAGCGCATGCCGGTGCGGATCAAGATTACAAAACCCCATCCTGACG
TGGTGATGCGTGATGGCGGCAGCGCAACCGTTACCATCAATACAGGCTATCATCGCACATTTGCTACCTTG
TGGCGCGATCTGACCGGCATGGTGGGGATCGAC
>BR1024
ATGAGTATCAACGCGACCACGCTCGACCGCCGCCTCAATGCCTATCGCCCCGATCTTGCCGATGAGCGGCT
GCGTGGTCAGGTGGAGGCATCCCGCTTCGTTGCGGGAACGCCGATGCAGGTTTCGGCTCCGGTCGTCGATC
TGCGCGCAGAACCGCGCATGGATAGCGGCCCGCAGACGCAGATGATCTTTGGCGACAAGGTTCGCGTTTTC
GAGGATATGGACGGCTGGTGCTGGGTGCAGGCCGAACGCGACGGTTATGTCGGCTATGTTTCGGCGGCTTC
ACTCGAAAGGCCGGACGGTGATGCAACACATATGGTCGTCGTGCCGCGCACCTTTGTCTATCCCGGTTCCG
ATCTGCACTTTCCGCACACAATGGCGCTATCGCTCGGCTCCCGCGTGAAGATTGTCGGCGAGGCCGAAACA
CGCGGCACGAAATATGTGCTTCTGGCAAGCGGCGAGGCCCTGATCGCCAGCCATGTCGCACCGCTTGGCCA
ATATGCTTCCGATTATGTAGCGGTTGCGGAAACCCTGCTTTACACGCCCTATCTGTGGGGCGGCAGTTCCG
GTTTCGGCATCGATTGTTCCGGCCTGGTGCAGCTTTGCATGTTTGTGGCAGGCCGGAATGTCCTGCGCGAT
TCCGACATGCAGCAGAAGACCCTTGGTGAAGTCGTGCAGCCTGATGGTGCCTACGCCAATCTGCGGCGCGG
CGACCTTGTCTTCTGGAAGGGGCATGTCGCGATCTGCACCTCGCCCGACATGCTGATCCATGCGAACGGCC
ACACCATGACCGTGACGCTGGAACCGTTGCAGGACGCCATCAAGCGCATTGCCTATCTCTACGACCTGCCA
ACATTGATCCGCAGGCCG
>BR1022
ATGAAATCACGGCGCATGTTCCGTCACAGAGGTTCAGTTGCAATTTTGCTCACCGCATTTCTGGCGGGGTG
CGGTACAACAACGCCGCTCGACACGTTCAACCTGTCCACGCCCCAGCCCATGGTGACCGTACCGAAGCGCA
AGAACTTGCAATTGCTGATCCCCGGACCAGCCGCGCTGAAAGCTCTGGATAGCGAGAATATCGTGGTCAGC
TCTGCGCCGGGCTCCATCGAATATCTCAAGGGTGCGCAATGGGGCGACAGGCTGACCAATATCGTGCAGTC
GCGCCTTGTGCAGGCTTATGAAAATACCAGCGCATTTGGCGGCATTGGCCGTCCGGGCGATGGGCTGGCGA
TAAATTATCAGGTTCTGACTGATCTGCGCATGTTCGGCATTCAGGCCTATACATCGCCACAAACTGCTGTT
GTGGAAATGGCCGTCAGGCTCTTGAGCGACAAGACCGGCGAAGTCCGCGCCACCCGCGTATTCCGCACGGC
AATTCCTGTTACGGGAACCAGCAATGCGGCCTATGTGAAGGCTTTGGACAAGGCTTTTGACCAGACTGTGA
ATGAGATCGTAAGCTGGACGATTTCAACCCTT
>BR1021
ATGTCCGCCGAAATCATTTCCCAAAAATCCGCCGCCAGCCGCCCCATCTGGTTTGTCGCCAAGAACGCGCT
GGATGAAGCCGCCCTTCCCGAAAGCGCCGCTGCCTGGGCGAAGGTCAATGGCTTCGATGGTGAAGCCGGGC
GTGTGCTGGTGCTGCCGGGGCGCGATGGCGCGATTGCGGGCGCCCTGTTTGGCACCGGCGAGGCGCCGGAG
AACGGCCAGCCGCAGCTTATCGCCGGAAAGCTTGCCCGCAACCTGCCGGAAGGCGATTGGCATATTGAAGG
CAAGCCGGGCGATGCCGGTCTTGCCGCACTCGCTTTCCTGATGGGCGGTTATAGTTTCACACGCTATCGCA
AGGCCAATGGCAAGACGGTCCGCCTGGCCCTGCCCGAAGGCGCGGACGCTTCTGAAACACGCCGTCTGGCA
GAAGCCGTCACGCTCGTGCGCGATCTCATCAACACACCCGCCAACGACATGGGGCCGGCGGCGCTGGAGCA
GGCCGCACGCGATCTGGCGGCACAGCATGGCGCGCGGGTGGCAACCACGGAAGGCGAAGCGCTTCTGGAAA
AGAATTTCCCCATGATCCATGCGGTTGGCCGTGCAGGTGCCGTGGCCCCGCGCCTGATCGACCTTCAATGG
GGGCGGGACAGCGATCCGAAGATCACGCTTGTCGGCAAGGGCGTCTGTTTCGATACGGGCGGTCTCGACAT
CAAATCCGCAAGCGGGATGCTGCTGATGAAGAAGGACATGGGCGGCGCGGCCAATGTGCTGGGTCTTGCCG
CACTCATCATGGATGCGAAACTGCCGGTACGGCTGCGCGTGCTCATTCCCGCGGTAGAAAACGCAATCGCG
GGCAATGCTTTCCGCCCCGGCGACATTCTGCAAAGCCGCAAGGGGCTGACCGTTGAAATCGGCAATACCGA
TGCCGAGGGCCGTCTGGTTCTGGCCGATGCGCTGGCGCTTGCCGACGAAGAAGAGCCGGAAATGCTGATCG
ACATGGCAACGCTGACGGGCGCGGCCCGCGTGGCGCTTGGGCCGGATCTTCCGCCCTTCTATACGCATGAC
GACCAGCTTGCGGGCGAAATCGCCGTAAAGGCCAGAGAGACCGCCGACCCTCTATGGCGGATGCCGCTCTG
GCAGCCCTATGCGCAAAAGCTTTCCTCGCGCGTGGCCGATATCAACAATGTCACCACGGACGGTTTCGCCG
GTTCCGTGACAGCGGCACTGTTTCTCGCCAAATTCGTCGAGAGGGCACGCCAATGGGCGCATTTCGACATT
TTCGGCTGGGTGCCGGTCGAAAAGCCTGCCTCCCCCGTTGGCGGCGAAGCGCAGGCCATCCGCGCCCTTTA
CCAGCTTTTGAAGGAACGTTATCCGGCCCGC
>BR1001
GTGTGTTGTATGAATTTTATAAAATTTGACATGCTTTATTTTTATGGAGCCGGGAAATACAAAATGCCGGG
GCGTTTTCCCGCCCCGGCATTTCATATCACGTCAAGTCAG
>BR0999
ATGCAGCGCCTTGGCAACGGCAGCGGAATCGTCAGTCGAACCGGCGCGTTCGATGCCGGCCTTGATGACTT
CCACAGCCGCATAGGCGTTCATCGTGAAAGCTTCAGCCGGGATGTTCTTCGCCTTCAGGGCTTGGATAGCG
TCCTTGGCAGCCGGGTTCTTCGTGGCATCCTTGGCGTTGGTGAAGAGCGTACCCTGTGCGTTTGTGCCGCC
GATTGCCCAATATTCGGTGTTGGAGAGGCCTTCGCCGCCGAGCACCAGGGCCTGCATGCCAGCATCGTGGA
GCTGGCGCGACAGGAGACCGCCTTCAGCATGGTAGCCGCCGAAATAGACCACTTCAGCACCGGCAGACTTC
AGCTTGGTGACGAGCGCGGAAAAATCCTTGTCGCCCGGTGTGACCGAATCATAATGGACTTCAGTGATGCC
GCCCTTGTTGATGGCCGCCTTGAACGCATCGGCAAGACCCTTGCCATAAGCGCCCTTGTCGTGGATCACGG
CAACCTTCTTGTCCTTCATGTTTTTCAGGACGTAATCAGCCATGACTTCAGCCTGCTGGTCGTCGCGGCCG
CAGGTACGGAACACGTTTTCCAGGCCGCGCGCGGTCAGGTCCGGGCCGGTAGCCGTCGGGGTGACCATGAG
GACGCCGTTTTCAGACAAAACGTCGGAAACGGGGACGGCAACGCCGGTCGTAACCGGGCCGACGACAAACT
TGATGCCATCGCCAACGATCTGGTTGGCAGCGGAAACGCCCTGCTTCGGCTCGCCCGCATCGTCGGCAAAC
TTCAGGACGACTTTCTCACCCTTGATGCCGCCAGCCTTGTTGATGACTTCCACGGCGGTTTCCGCGCCCTT
TTTCACCTGATCGCCGAAAGCGGCAACCGGGCCCGTCAGCGGTGCAATCACACCGATGGTGATGTCTGCGT
AAGCGGCGCTGGCAAAGCCGATGGTAGCGGCAAAAGCCAAGCTGGACAGAAGTTTCAGGTTCATTCTTTTT
CTCCTTTGGTGGGGCGGCATTGTCCGCACAGCCCCGAAACAACCCATGCCGGTCAATTTTCAGCGCCTGCG
CGCTGCCGAATTGCCGGGCCCTCCCTTTTGTTCATCGCG
>BR0990
ATGTTGATCGTCGGTGCCGGTATATCTGGTTTGGCTTTGGCGCGTGCGCTGCGGCAATTCGATATTGAAAC
GCGCATTCTCGACAAGCGCAATCAACATGCAGAAGCCGGGTTGGCCATCAATCTACCGGGCAATGCCATTC
AGGCACTGGAACGTCTTGGCCTGAAAGAACAGGTTGATGCGCTTGGATATCCGACGAAGCGGCGCGAATAT
CGGACAGCAAAGGGGAAATTGCTGTTTTCCGTCAACGAGACCGATTTCTGGGGCGAGCGCCATCAGCCGCG
CTGCATCCTGCGCTCGGACTTGCTGCGCATCCTGCAAGAAGGCAATTCGCAAGACAATCTTCTCTATGGCA
AGAAAGTCGTTGAAATCTCGCAAGATGCCGGGCGCGTTTCGGTGGCGCTTTCCGACAATGAAACAATGGAG
ACGGCGTGCCTGATCGGCGCGGATGGCGTCAATTCCATCACCCGCACTCTCGCATTcAATGGCAATGCCGT
CAGGGCGGCAGCTTTATCCAATTTTAGCTGGCGCTTCATGGCGCCAAATCCGGGCGTCGATTGCTGGTCGC
TCTGGGTGGGCTCAAAAAGCATGATCCTGCTTATTyCGGTGAGCGAGCAAAGCGTTTATGTtTGGGCGACA
CTwmCCGGAAACCGGACcGAtAGCGGCCAGGTGCGCCTTAGCGGCGAGCATTTTTCCGATTTCCCCTCCGA
TCTGAGGTCGATCATCGAAAATGCGGTCAAGACGCCCCAGGACGTCTATAATTCTCCCTTGCAGGAAGTCC
GCATGGATCGCTGGTCAGCCGGAAACGTATTGCTGCTCGGCGATGCCGCCCATGCCACGGCACCCGTCTGG
GCGCAAGGCACAGCACTTGGCATGGAAGCCGCCCTCACCCtTGCAAAACTCGTTTCGCAAAAGGTGGAACA
GCCGGAGCTTTtCGACCAATTTGAAAAGCTGCATCGTCCGCGCGTCAACCaTGTCGTCGCCATGACGGACA
AGATGTCCAAGGCAGCCAAGATTCCGTCCTTTGCGCAAAAACTGCTCCTGCCCATTGTCGGGCCGCGCAAT
TACGAGGCAACATACGGCCCCTtGCGGGACGGCAATTTC
>BR0982
ATGAATAAGCTCGGCGTGTTTATCGGCTATAACCCAGGCCAATTAGATCCGTATCAGGGTATTTCTCGCTT
AATTGCATTCGTGATCAAGGGGGCCTTGAACCAGGGTAGCGGTGTAACAATTGCTTGCCCCGGCTGGCTAA
AGGACGATGTACGTGTTCTTTTGGAAGATGCTGATATCCCACTTGAAGCGGTCAAAATTATCGCGACGAAT
GGTCAGCCTCCATTGGCTTCGTTATGGAAGTTGAGAGATAAGTTCCGTAAGAGACGGACGAGTAAACGAAA
ACGTCTCTGGCTGGAGCGCTATGGCAAAAATGTTGCAAATTTTGTTGCAGAATGGCTTTCTTCGCGCTCGT
ATTGGGGGATTTTTTTGGGGGCTGCTGCAATTGCTGTAGTGACTATTCTACTTGCCGTACCAATTGCTATA
GCCTTCACCGCTCTTATCGGCCTTCTATTTGCTCGTCGGCTTATTAGACGTGTTATCAGGTCAAAGCTTGG
TTTGTTTTTTCACAAAAATGCCAATCAATTCAACAAATTAATGTCATCTGATGAAACCATCGACCAGATGA
GGGAACGGGAATTCTCATTGTTGATGAAGAAGATCAACGCCCAGAAGGACATCAAAGTTTGGTATGTACCA
GCCATGTTTTGGCCCGAGGTCGCTAACATAAAATCCAAGATCGTGATGGCGGCCCCGGATATCGTCTTCTT
CGATTACCCAGGGAACTTCCGAGGGATACGAGAGCATAACTCATACGATCGCATGTTAAAGAGCTTGAGGT
CTGCCGATCATCTCGTTTGCTACAGTGAAAATGCGAAGCAAAAACATTTTGTTGAACGGTGTGATGTTCCT
GCGGAAAAGATAACGGTTATCCGTCACGGGTTCGTAGATTTGGGAGCTTCTGGTGCGGCTATTTTGCGTCA
AGATGCACTTGACACATTACATGCCTTTATCAAGAAGAATGATGGTCGAATGCCGGAATATTTGAAAGGTT
TCCGGTTTGACGATGTTCCTTTCTTCTTCTATTCTTCACAGCTTAGACCACACAAAAACATAGAAGGCCTT
ATTAGGGCTTATGCAAAAGTATTGAAAGAGCATCAGCGCCCAGCAAAACTTATCCTTACGGCACAATTTCA
ATATGACAAGCGCATCCAGACCTTTATAGATGATAATGGCCTGCATGCTGATGTGCTGTCGCTGCATTCTG
TACCAAACAAGGTACTTGCTGCTCTATATCACCTGGCATCATTGTCGGTCACGCCAACAAACTTCGAGGGA
GGGTTCCCATTTACATTTTCAGAAGCCTATTCGGTCGGCACTCCATCGATCATGAGCCGTATCCCAGTCGT
GCAGGAAGTGATTGATGATCCAGAGCTTCAAGACTTGATGACATTCAATCCTTTGGATGTGGATGATATAG
CCAATAAGATGATTTTTGGTTTGGACAACAGGCAACGTCTATTTGAGGCGCAGTCTAGTCTATACGCCAAA
TTATCCGCTCGCACATGGCAGGTCGCCGCAAATGATTATCTGTCTCTGCTTAAATCTGTTGCAGAC
>BR0971
TTGCGACGTCAGGCCGGTGCTCGTTCCACGATCCCACGTAACAGTCGCAGCCGCAGACCACTGGTCATTGA
ACTTATGACCGACACCGCCGCTAACCGTCCAACCATCCTGATAGTAAAGGTTCAAACTGGTGATCTTCGTG
CCTTTGGGAGCGATCTTGTTATCAGAAGAAACAAAGTCCACCGATGTGATGCTCGACCAGTCGGTCCACTT
GACCGAGCCGAATGCGAGCCAGTCCGGGGCGATACCGGTCTGGAACTTGAATTCAACCGACTGCGGCGTAG
CAACATCGCTCTCCACATTAAGCGGCATGCCCCGCCCCGTTATCGGGTTCAGCACAAGATTGTCGATCGTG
CCTTCAAGGTCATACTTAACTTCCGATTGATAGACGAGCGTGGCGCGCATCGCATATTCAGGAATTTCGTA
AGCAGCACCAAGACGCCAGCCAACCAACTGATCTTCAGCATCAAGCGATGCTACCCGAACCGGCCCTGGAA
AAGGAAACTTCGTCGGATTCATGTTTGGACTAATGCCAGGAATCAGACGGGTCTGCTCACCCTTCAGTTCC
TGATAGCTGACGCCGCCAAGAATTCGGAAATAACCTTTTTCCCCTGCTACAAAGCGATAGGAACAGTTCAC
ACCATAATCATGAGACGAAATCTTCTGCTCGATTGCGGTGAACATGCGAACTGTGTCGGTACCGACATCAG
TCTGGATACCCCAGGGCTGACGATATTGCGCCGCACAGGCAAGATCGCTGGTGATGTCGAATTTGGCGGAA
ACCTTTGGAACCTAGTAGCTCTTGGCCTCATCGACTTCTGTTTCATACGGACGCCCGGTCGTGGGATTAAT
GCCAGCCGGAACAAAATTCTGCCCCCCGGTCCACTGAGGGGTTTTTTCCATAGCAGAGCCGCGAATATTCT
TCAGCTTGCGTTGTGGCGCAACGAAGGTTCCGCCAGCTTCGACGGCGTTACCATTTTCAAAAAGAATATCG
AAATCCTGCGAGCCACGCTCAAACCCACCCGCATGAGCCATAACCGTACTGCCCAGCAAAGCAGCTACGAA
CCCAGCTATTTTCACGCGTCCCATCTTCATGAATGTCCTCCCAGACAGCAAAGGCAT
>BR0944
TTGAACTGGCTGACGGATTACAAGATACCTGTCGGGCCAACCGCCAAATCGGTGGTGGACTGGCTGACGAC
CCATCTGGGACTTTTCTTCGACACTCTGGCCGCAGTCATGCAGAGCTTGATTGACGATTTGCTGTATCTCC
TGGAACTCCCTCACCCCCTCCTTCTGATCGCCATCTTTGCAATCATTGCATGGCTCATTCAGCGTCGTATC
TCGGTCGTTCTGCTCACCATTCTGGGTTTCCTTTTCATCATCAACCAGGGCTATTGGGAACCCACCCTGAA
AACACTCACCCTCGTTCTGTCATCATGTTTTGTCTGCATGGTGATCGGCGTACCACTTGGAATCGCCGCAG
CCCATCGCCCGGCCCTTTACGCCGCGATGCGCCCTGTGCTCGACCTGATGCAAACGCTGCCGACTTTCGTG
TATCTGATCCCGGCGATTGTTTTCTTTGGTATCGGCATGGTGCCGGGTCTTCTCGCCACCGTCATCTTCGT
GATCCCTGCTCCTATCCGCCTGACCCAGCTCGGCATTTCCTCAACACCCGCCTCGCTCATCGAAGCCGGCG
AAGCTTTCGGCGCGTCACGCTCGCAGCTTCTCTGGAAGGTTGAACTGCCCTACGCGATGCCGCAAATTCTG
GCCGGCCTCACCCAGACGATCATGCTGTCGCTTTCGATGGTGGTCATTGCAGCCCTTGTCGGTGCCGATGG
CCTCGGCGTGCCTGTGGTTCGCGGCCTTAACTCCGTCAATATTTCGCTGGGTTTTGAAGCAGGTTTCATCA
TCGTCGTGGTCGCCATCGTGCTCGACCGTATCTTCCGCGCCGGACGTGAGAAA
>BR0936
ATGACCACATTGACCGCGAAATGGAACTTTCCGACCACTGTGCTTTTCGGGCCGGGGCACATCAAGGAGCT
TTCAGCCGTCCTGAAAGCCGCAGGCATCGAGCACCCGCTTTTCGTCACCGATCCGGGCCTCGCCAAACTGC
CTGTGGTCGCCTCGACATTGGGCCGTCTGGTATGCGCGGGCGTGCATNNNNNT
>BR0917
TTGCAAATTTTCGTCTTCGGACGTTCGTGGATCGGGGCGATTGCCCTTCTGATGCTCATATGCGCTGTTGC
GCGCTTTTTCCTGCCTGTCGGAAAGCGCAGTGGCGTTCTCATCGCCGCAAGCCTGGTCGGGTTCATTTTTC
TGGCTTTGCAAGGATTGGCAATTGGTTTTTCCGGCTGGAACTGGACCATCAGCGAAACGCTTTTCGGGCCG
CTTTCCGATGGCCAGCCATCTTTCGGCGCGGGCGCGATTCTGACAGGCCTCTGCTTTCTCCTGATCTTCTC
TTTCGGGCTTGCCGAGCGCGGTGTGATGAAAGGCGACGCCTTTGTTGTTTCATCGATCACGCTTCTGGTGG
CGCTCGTCGGCGTCTTTGTCTTCTATCCCGTGCTCAGCATGTTCGTTGGCTCGGTGCAGGATTTCGACGGT
TCGTTCAATCCGGATGGCTTTATCGGCAATATTCAGGATTCCTCCATCTGGAGCCTTGCCTGCGTGGTGGG
CGAGGGGCGCTGCGGTGTGGCATGGCGCACGCTTTGGCTGGCGCTGATGACGGCGACCGGTTCCACGATTC
TGGGCCTTGCCTTTGCTCTGGTCGCGACCCGCACCAACTTTCCTTTCAAGAAAGGGCTGCGGCTTCTCACC
ATTCTTCCGATCATCACGCCGCCCTTCGTGGTGGGCCTTGCGCTGACGCTGCTCTTCGGGCGTGCGGGCGT
GGTAACGGAACAGATTTCCAGCCTCTTTGGCGTCGAGCCGGGCCGCTGGCTCTATGGCCTCACCGGCATCT
GGATCGCGCAGGTTCTTTCCTTCACACCCATCTCTTTCCTCGTGCTGATCGGGGTTGTCGAGGGCGTGTCT
CCCTCGATGGAGGAAGCCTCGCAGACGCTTCGCGCCGACCGCTGGCGCACCTTCAAGCGCGTTTCGCTACC
GCTGATGGCGCCGGGTCTCGCCAATGCCTTTCTGATTGCCTTCATCGAAAGCATGGCCGATTTCGGCAACC
CAATGGTGCTGGGCGGAAGCCATGGCGtTCTTTCGACCGAGATTTTCTTCGCCGTCGTCGGCGCGCAGAAw
GAcCCCTCGCGCGCGGCTGTTCTCGCCATTGTGCTCCTTTGCTTCACCCTGTCGGCCTTCCTCGTGCAACG
GCTGTGGCTTTCGGGCAAGAACTTCGCCACCGTCACCGGCAAGGGcGATTCCGGCATACATGCTGGCTTGC
CGCGCGGGTTGAAGATCGGCGTCTATGCGCTTGTCATTCCTTGGATGTTCTTCACCGTGGTCGTTTATGTG
ATGATCCTCGTTGGCGGTTTTGTGCGCCAGTGGGGCCTCGATAATTCGCTGACGCTGGAACATTATGCGCG
CGCCTTCTCCGTCTCATGGACCGATGGCGGGCTGGCCTGGACCGGCGTTGCGTGGAACTCGTTCTGGACGA
CGATGGAAATCTCGCTCATGTCAGCGCCGCTCACTGCCGCGGTCGGCCTGCTCACGGCCTATCTGATCGTG
CGGCAGCGTTTCGTCGGGCGCAATGTGTTTGAATTTGCACTGATGCTGAGCTTTGCTATTCCGGGCACGGT
CATCGGCGTCAGCTACATCATAGCGTTCAACCTGCCGCCGCTGGAAATGACTGGCACGGCGCTGATTCTCA
TCGCCTGTTTCGTGTTCCGCAATATGCCTGTGGGCGTGCGCGGCGGCATCGCGGCGATGAGCCAGCTCGAC
CAAAGCCTCGATGAAGCCTCGCTCACGCTGCGTGCGGGCAGTTTCCGCACCATCCGCAAGGTGATCCTGCC
CTTGCTGCGTCCTGCCATCACAGCGGCGCTGGTCTATTCCTTCGTGCGCGCCATCACATCCATCAGCGCCG
TGATCTTCCTTGTTTCGGCCCAATATAATATGGCGACTGCCTATATTGTCGGGCTGGTGGAGAACGGTGAA
TTCGGCGTGGCGATTGCCTATTCCTCCATGCTGATCGTGGTGATGGTTGTCGTCATAACCAGCTTCCAGCT
TCTCGTGGGCGAACGCAGGCTGCGGCGCGAAAACCGCGTTGCGGCGGCTGCGCCCGTCAAATCCGTCCGTC
AGGAGAAAACCGCA
>BR0916
ATGACTGCCCATTCCCGACAGGACCTCGAAACACGGCTGGCGCTGGCCGAAAAGATCGTGCAAGAGGCGGG
CGCCAAGGCGCTCGACTATTTCAATCGCCGTGAAACGCTGGTCATCGAAACCAAGCGTGACCCGCAGGACG
TGGTATCGATCGCCGACCGGGACGTGGAGCAGCTTATCCGCGCCCGTGTGTCGGAAAGCTTTCCGCAAGAC
GGGTTTCTGGGCGAGGAATATGGGCTGAATGCCGGTTCGTCCGGCTATACCTGGGTGGTCGATCCCATCGA
TGGCACCAGTCCGTTCGTCAACGGTATGCCCAACTGGTGTGTTTCCATAGCTGTGTTGAAAGATGGCGAGC
CGGTGGTGGGCGTCATTTTGGCGCCATGCTTCAACGAGCTTTATGTCTCCGCCAAGGGGCAGGGCGCGACG
CTTAACGGCAGGAAACTTGCGCTCGACCCGTCGCGCAATATCCGCAACGCGGTGACGGGCATCGGCGCGAA
CAATTACGTCACGCCGCAACTGGTGACCAAGGTCGTCGAGAACCTGCTGGAAGCGGGCGGTACTTTTATCC
GCAACGGTTCCGGCGCGCTGATGATTGCCTATGTCGCGGCGGGCAGGCTGGTCGGCTATTACGAACCCTAT
ATGCACGCCTGGGATTGCATGGCGGGCTTCTGCCTTGTGCGTGAGGCTGGCGGCTATATCTATCCGTTCCC
GACAGATGGCGAAAACCTCACGAAGGGCAACCGCGTTTTCGCCGCTGCACCGGGCGCGGTAGACGACCTGA
AGAAAGTCGCTGGCCTA
>BR0912
TTGCGACGTGTGGGACAAGGCGCGCGCTTACTGAACACTTTCCCTCGGGGGAAGGATGGAGGAAATATGAA
AACCGTTCGACTGACCGCCGCGCAGGCACTGGTGCGCTATCTGGCCAACCAGATGACGCCGGAGGGCGACA
CATTCATCGCGGGCGTCTGGGCCATTTTCGGGCACGGCAATGTGGCGGGGCTGGGCGAGGCGCTCTACGGC
GCTCGCGAGCATATGCCCACCTGGCGCGGCCATAACGAGCAGACCATGGCCCATGCGGCCATCGCCTATAC
CAAGCAGCTTGGCCGCAGGCGCGCCTGCGCCGTCACCTCCTCCATCGGCCCCGGCGCAACCAATATGGTGA
CAGCGGCAGCACTTGCCCATGTGAACCGCCTGCCCGTTCTGTTCGTTCCGGGCGATGTCTTCGCCAATCGC
GGTCCCGATCCAGTTCTCCAGCAGATCGAGGATTTCAACGACGGCACCATGACCGTCAACGACTGTTTCCG
CCCGGTAAGCCGTTATTTCGACCGTATCACCCGGCCGGAACAGCTTCTCACCGCCCTGCCTCGCGCCTTTC
GCACCATGACCGACCCCGCCGATTGCGGCCCGGTGACGCTCGCCTTCTGTCAGGATGTGCAGGCGCAAGCC
TATGACTGGCCGGAAGAATTCTTCACGCCCAAGGTCTGGCACTGGCGCCGCCCGCCGCCGGACGAGCGGGA
ACTGGAAACCGCTGCCGCCGCCATCAAAACCGCGCTGAAGCCCGTCATTGTGGCGGGCGGCGGCGTGCATT
ATTCCGGCGCGCATGAGGTGCTGCGCGCTTTTGTAGAAACACATGGCATCCCCGTTATTGAAACACAGGCG
GGCAAATCCGCACTGCCATGGGACCATCCGCTGAATTTCGGTCCTGTCGGCGTGACCGGCGCTGACAGCGC
CAATGCGGTTGCGGCAGAAGCCGATCTGGTGATCGGTGTCGGCACACGCTTTCAGGACTTCACCACCGGCT
CATGGGCCCTGTTCAGGCATCCGGGGCGCAGGCTTCTTTCGCTCAATGTACAGCCCTATGACGGAGCCAAG
CATAATTCTCTGCCGCTGGTAGCAGATGCGAAGATTGGGCTTGAGGCACTCTCCGGGGCCATTGGCGCTTA
CAGACGCCAGGCCGTCGATGCCAGCCTCAAAAACAGATGGTTTGCCGCTGCCGACCGTTTCACCGCCGCGC
CCAGCGACGGCAATGCGCTACCGACCGACATGCAGGTCATCGGTGCGGTGCAGCGGCAAGCGCGCGAAAAT
TCCGTTGTCATGTGCGCGGCAGGCACCATGCCGGGTGAGTTGCATCAGCTCTGGAAGGCCGGGCGCCCCAT
GTCCTACCACATGGAATATGGCTTTTCCTGCATGGGCTATGAGATTGCCGGCGGCCTTGGCATCAAGATGG
CCGAACCGGACCGCGACGTCATCGTGATGGTGGGCGACGGTTCCTATATGATGGCCAATTCGGAACTGGCC
ATCAGCATCATGATGGGGCAGAAAATCACCGTCGTCGTGACCGACAATCGCGGTTTCGGCTGCATCAACCG
GCTGCAAATGGCGACCGGCGGCGCGGAATTCAACAATCTGCTCGACCATGCCGCCCATGTGAACCCGTCGC
AGATCGATTTTGCCGCGCACGCCGCCGCCATGGGTGCGGATGCGAAAAAGGTCGGCTCCATCCGCGAACTG
GAGGGCGCACTGGCCGAAGCACGCGACAGCCTGCGCACGACCGTCATTGTTATCGACACCGACCCTTACCC
CACGCCGGAAACCGGCGGCTGGTGGTGGGATGTCGCCGTACCGGAAGTCTCGGAACGCGATGAGGTGCGCG
CCGCACGCAAAAACTATGAAGCCCAGATCAAAGAAAGAAAC
>BR0893
GTGGCAATTGCGCAAAACTGGCTCTATATGCGGCTGATAGATAATTCAAACTTCGGCTTCAGCATATGGAC
GCACGCAATCTGCAAGATGAAAGGCCCGGCGAGGAGAAGTGCCGGCAGCATTCCGGCACGGCAT
>BR0891
ATGGGACCGATCACGGATAAGGTGGCGACAGCCGAAAGTCTGCCACAGGAAACCAGGATTGCCATTATCGG
CGGCGGCGTGATCGGTGTTTCGACAGCCCTTTTCCTTGCCGAGCGCGGCATTCCCGTCACCCTGTTTGAAA
AGGGTGAAATTGCCGGGGAACAATCGAGCCGCAACTGGGGCTGGTGCCGCCGCACCGGGCGCGACAGCCGC
GAAATGCCTTTGATCGTGGAAAGCATGCGCCTTTGGGAAGGCATGAATGCACGCACAGGTCGCGAAACGGG
ATTCAGAGTAAAAGGAATTGTCTATGCCGCTGAAAAAGAAGAACAAATGGCTCGTTATGCCGAATGGATCA
AGATTGCCCGCCAGCACGGCATCGAGACCCACCTTCTGAACGGCGAACAGGCTGAGGCGCTTGCGCCGGGC
CTGACACGCCGCCTTGCGGGCGGCATGTCAACACCGCTCGACGGACGTGCAGAACCCCAGAAGGCGGTTCC
TGCAATGGCAGCAAAGGCGCAGGAAGACGGTGCTGTCATCCTGCAGAATTGCGCGGTGCGCGGCATCGAAA
CCACCAATGGCCGTGTGAGCGCTGTGCTAACCGAAAAGGGCCGTGTGCGCTGCGAGGCGGTCGTGGTTGCA
GGCGGCGCATGGTCTCGCCTGATTATTTCAGGCTTCAAGGCCCATCTGCCGCAACTCAAGGTGCGCTCCTC
CGTTTTCCGCACCGCGCCTATCGAAGGCGGCGTGGAACCTGCCATTGCCTTTTCCGATTTCGCGCTGCGCA
AGCGGCTCGACGGCGGCTACACGGTGGCGAGCCTCAGCAGCTCCATCGCCGATATCGTGCCGGACAGTTTC
CGCTTCTTCCGCGACTTCATTCCCTCGCTTTCGACGGAATGGCGTTCGCTGCGCTTCCGCCTCGGCGAACG
CTTCTTCGAGGAAGCCTTCCAGTGGAAGCTACGCCCGGCAGACCAGATGTCCATCTTCGAGAAAATCCGCA
CTCTGGACCCCGCACCGCACCATACGGCCAATGTGCAGGTGCTCGCTCAGCTAAAAAAGGCAATCCCGGCT
TTTGCGCCAGCCGTCATTGCTCAGGAATGGGCCGGGCTGATTGACACCATGCCGGATATCATTCCGGTCAT
ATCACCACTTCCGGGTGTCGAGGGCCTCATCGTGGCGACCGGCTTTTCAGGCCACGGCTTCGGCATAGGCC
CCGGCGCGGGGCGTCTCGTCGCAGATATGGTCTGCAACGAAACGCCGCTCGTCGATCCCTCTCCGTTTCAT
ATCTCCCGCTTTTCCGACGGAACAAAAATCCGCATTGAAAACTGGGAA
>BR0890
GTGATGAAGTCCTTATTTATTGCATCGACAATGGTGCTTATGGCTTTTCCGGCTTTCGCAGAAAGCACGAC
GGTAAAAATGTATGAGGCGCTGCCGACCGGACCGGGTAAAGAAGTTGGCACCGTGGTCATTTCCGAAGCCC
CGGGCGGGCTGCACTTCAAGGTGAATATGGAAAAGCTGACGCCGGGCTATCATGGCTTTCATGTTCACGAA
AATCCAAGCTGCGCTCCGGGAGAAAAAGACGGCAAGATCGTACCGGCTCTTGCTGCCGGCGGGCATTATGA
TCCGGGTAATACCCATCACCATTTAGGGCCTGAAGGTGATGGACATATGGGCGATTTGCCACGCCTGAGCG
CCAATGCTGACGGCAAGGTGAGTGAAACCGTTGTCGCTCCACATCTCAAGAAATTGGCGGAAATCAAGCAG
CGTTCTTTGATGGTCCATGTCGGAGGGGATAATTATTCCGATAAGCCTGAGCCGCTTGGTGGCGGTGGTGC
CCGTTTTGCCTGCGGCGTGATCGAA
>BR0888
ATGACTGTCGCAGAAGCGGTTGCCCCACAGGCAAACTCCGTGCGTCGTGTCTTGACGGCAAGCATGATCGG
CACGACCATTGAATTTTTCGATTTCTATATTTATGCAACGGCTGCGGTGATCGTATTTCCGCACCTGTTTT
TCCCGGCAAGCGACGGCAATTCCGCACTGTTGCAATCCTTTGCCACTTTCGCCATCGCCTTTTTCGCCCGC
CCTGTCGGCGCTGCGATTTTCGGCCATTTCGGTGACAAGATCGGCCGCAAGGCCACGCTCGTCGCGGCACT
GATGACCATGGGTCTTTCCACAGTTGCCATCGGCTTCCTGCCAACTTATGCCTCCATCGGCGTTGCGGCTC
CGCTTCTGCTGGCGCTCTGCCGTCTGGGTCAGGGGCTGGGTCTTGGCGGCGAATGGGGCGGTGCAGTGCTG
CTCGCCACCGAAAACGCACCGGAAGGCAAGCGGACCTGGTATGGCATGTTCCCGCAGCTTGGCGCACCGGC
AGGCTTCATTCTGGCCACGGGCATTTTCCTGCTGCTCGCCGAAACCATGACCGAAGAACAGTTCTTTGCCT
ATGGCTGGCGTGTTCCCTTCATCGCCAGTGCAATTCTGGTGGCCGTGGGCCTTTTCATCCGCCTGCGGATT
GCCGAAACACCGGAGTTCCAGAAAGCCATCGACAAGGCTGAAAGGGTCGCCGTGCCAGCCGCACAGCTTTT
CAGGCATCACAAGATGAACCTGTTCCTTGGCACCATCGGCACCATGGCAACCTTCGTGCTCTTCTATCTCA
TGACCGTGTTTTCGCTCGGCTGGGGCACGCGCGCACTGGGCTACAGCCGCGAGGAATTCCTCGTGCTTCAG
ATGATCGGCGTCATCTTCTTCGGCCTCACCATTCCGCTTTCCGCCCTGCTCTCCGACCGCTACGGCATGCG
CACCATCATGGTCATCGTCACCGTGCTGATCGGCCTCTACGGCTTCATCATGGCGCCGCTCTTTGCGGCGG
GCACCGCAGGCGTGTTGGGCTTTCTCATCATCGGCTTTGGGCTGATGGGCATGACCTATGGCCCCATCGGC
GCGGTTCTTGCCGAACCCTTCCCGACTTCGGTGCGCTATACGGGCGCGTCGCTGGCCTTCAATCTGGCCGG
TATTCTGGGCGCGTCGCTTGCCCCCTATATCGCCACCTGGCTCGCCACCGATTACGGCTTTGCCTATGTCG
GCTATTACATGGTGGCAGCGGCAATCATTTCGCTGATCGGCTTTGTCTTCATAACGCTGAAAGACAAGAAA
>BR0882
ATGCTGGCGCGTGAACAGATGCAGCGCAAGCTGAAGGGCCGCCGCTCcAATyCCCGCCTGCCGCAACTGAT
GGAACTGATCCTGCGCACTCCGCtGTTTCAAGTCAACTGGTCGAAAAGGAATTGCAGGTCACACAGCAGGG
CGCGC
>BR0872
ATGCGTAGTCGCAGTTTTTCATGTTCCGCTATAGCGATGGCTCTAGGCTTGGTCCTTACCTTGGCACTTGC
CTGCCATAGTACTTTTGCTTTCGATCTGAACGACGATTCAGAAAAATCGCGAAGCCCCTTCGAGCTTTTCA
AATTCGGCTTCTCTGCCTACAAGAACGGGCATAAGGACGAAGCCATCAAGGCTTTGCGTTATGCGGCGGAG
CGTGGCCATCAGGGCGCGAAGTGGAAGCTGGCGCGTATGTATGCCGAGGGCGACGGCGTGGCCGAGGATGA
TTATGAAGCCTATAAGATTTTTGAGAAGATCGTTCGTGAAGGCTCCGAACCCGGCTCCGAAAACGCTCCCT
ACGTCGCAGATGCGCTTGTCGCGCTTGCAGGCTATGTGAAAAACGGCATTCCCGGCTCGCCGGTTCAGGCC
AACCCCAACATGGCGCGTGAACTTTATGTGCAGGCAGCGGCCAATTTCGGCGACTCTATTGCTCAGTTTGA
GCTTGGCAAGATGCTGCTCGACGGCGAAGGTGGTGAGCGCAACGCTGTTCAGGCTGCACGCTGGTTCCAGC
TTGCGGCCCGGAAGGGCAATACCGGTGCGCAGGCCATGCTCGGCAATATGCTGTTTCAGGCCGGCAAGACC
GTGCGTGGCCTTGCCATGCTCACGGCTGCCTTCGAGCGATGCCCGGCCGAAGACTGCACCTGGATACGCGA
CATGCAGGAACAGGCCTTTTCCATTGCAGGCGAGGCCGACCGCCGCAACGCCATCGCGCTTGCAAGTGATT
ACGCTGTAAAAGGCAGTTAC
>BR0838
GTGCTGACAGAAGCGATTTCATGGGGCGTCAACTTTCATTCTGGAAATAAACGGACACGGGGCCGTGCACA
CCGGCGCGGCACGGCCGGTGCGGTAGAGATCGGGATATGCCTTGGAGGGTTTCCCCCCGACCCGCCCGGCA
AA
>BR0815
TTGGGATTGCTGGAAACGGCCATTACCGGGCGCAAGCGGCATAACAGCCACGCGGTCGTGGtGGGTGAACT
GGGGCGCGGCATCGTCGCGGGGACcATTCCCGAAgGGTCCATCCTTCCGGgCgATCATGAATTGTCGCTGC
GCTTCGGCGTATCCCGCACCGTTCTGCGCGAGGCGATGAAAACGCTTGCCGCCAAGCGCCTGATCGAGCCA
AAAGCCAAGGTGGGCACCCGCGTTCTGGGCCATGCAAGCTGGAATTTCTTCGATCCCGATGTCCTGTCATG
GCGCTGGAACCGGCTGCTGCTGCCGCCGCCGAGCGCGCCACCAGCGACGAGATCGTGGAGCTTTATGCACT
TGCCGCCAAATTCGACAATCCCGACCACACGCCGGAATCCATCGCCAAGGTCGATCTGGAATTCCATCTTG
CCATTGCCCGCATGTCCGGCAATCCTTTCATGCGTTCGGTCAGCGGCT
>BR0799
ATGAAAAAGCTGCTTATTTTGGCCGTTGGGATGATGCTGGCTGGCTGCATTACTGATGCTGGAAAACTGGA
AGATCGGATTGCCGCTCAAAGAGCGCCTTCACCGGCCATAAAGCAACTCATCATCAATGAGGCGCGGAATA
TCCTGCTCGATCCATATTCGGTCCGCGATGTCTCGATTTCATCCCTTATTCCGAATCTGAAAAGCTCCAAT
AATGATGGGTTCATATGCGTCCGCTTCAATGCAAAGAATAGCTATGGCGCTTATACGGGTCTAAAAACGGT
CGGAACAAACGTCATTAGTGGGCGGCTACTTGGATATTTTACCAATCCAGCCATATGCGCATATCCTACCG
TGAAGTGGCACCCCTTGCCGGAAGTTTCGCAACTGCGCAAACTG
>BR0781
GTGGATGCCATAATGATGGAGCATAGGAACGATTTCTGGGTCTTCGGCTACGGATCGCTGATGTGGCGGCC
GGGCTTTGCACATGTTGAAACGGTGCGTGCCCGCCTGCATGGCTATCGCCGCAGCCTGTGCATCTATTCCC
ACGTTCACCGCGGCACCCCCGACCATCCGGGTCTGGTGCTCGGCCTTGATACGGGCGGTTCGTGCCTGGGC
ATCGCCTTTCGCGTGCCGGGCGACATGACAGACGAGGTTATGACCTATCTGCGCGAACGCGAAATGGCGAA
CAAAGTTTATCACGAGAAAAAGCTTCATTTGCGGCTTGCCGATGGACGCATCGTTGAAGCCGTCGCCTATG
TTGCGGATCGCCGCCACAGGCAATATGCGGGCCGCATGACGAGTGAGAAAGCAGCCGCCATCATTGCTTCG
GCACAGGGCGAATCCGGCGCAAATATCGACTATGTTACCAATACGCTCATGCACCTGCGCAATATGCGCGT
TCGCGATCATGCGCTCGAACACATAAATGAGCTTGTTCTGGCTGCGGCAAGCCGGCAGGCAAACAATGCAG
CC
>BR0777
ATGTCCACAATCCACTTCGATAAAATCACGCTGATCGGTATCGGCCTTATCGGTTCCTCGCTGGCGCGCGT
CATTCGCCGCGAGGGCCTGGCAACGCATATCGCCATCGCCACCCGCAGCGCCGAAACCCTGAAGCGCGCCG
AGGAATTGAACCTCGGCGACAGCTACACGACCAACAGCGCCGAGGCCGTGAAAGATGCCGATCTCGTCATC
GTTTCGGTTCCCGTCGGATCCTCAGGCACGGTTGCGCGCCAGATCGCGGGCAATCTCAAGCCCGGCGCCAT
CGTCACCGATGTCGGCTCCACCAAGGCTTCCGTCATTGCACAGATGCAGCCGGAACTGCCGGAAAATGtGC
ATTTCATTCCCGGCCACCCGCTGGCCGGTACGGAATATTCAGGCCCGGATGCGGGCTTTGCCGAGCTTTTC
ACCAATCGCTGGTGCATTCTCACGCCGCTTCCCGACACGGATGAAAAGGCGCTGGAAAAACTCACCGCCTT
CTGGACAGCCTGCGGCTCGCGCCTCGACCATATGGACCCGCAGCACCATGATCTGGTGCTGGCCATCGTCT
CGCATCTTCCgCACATCATCGCCTACAATATCGTCGGGACGGCCAGCGATCTGGAACTGGTAACGAAGTCG
GAAGTCATCAAATATTCCGCATCCGGCTTCCGCGATTTCACGCGCCTTGCTGCATCCGACCCAACCATGTG
GCGTGACGTCTGCCTGCACAACAAGGATGCCATTCTGGAAATGCTGGGCCGTTTCTCGGAAGACCTTGCCT
CACTGCAACGCTCCATCCGCTGGGGCGATGGCGAAGCCCTGTTCGATCTCTTCACGCGCACACGTGCGGTG
CGTCGCGGCATCATCGATGCCGGTCAGGAAGTGGATGCACCGGATTTCGGCCGTCAGGCAGCCGTACGCTC
CACGGCCCCCCTCAAAGCGCCGGAAGGCGACCCAGCGGAA
>BR0706
TTGTGTCATTCGCAATCGATCCGTCTATCTGCCGATGTCGCCGCAGCTTTCCTGAAGGTATTTGCCATGAC
GATCACGCAATCCGGTTCTCCACACTCCATTGCCGGCAGAAAACAAACCTCCCGCCCGCTTGAAGGCGGCG
CAACCTTTACGCTGAAGCATCGCCTGCAGCGGCTTTTGTGGCAGGCTGCGTGGCTTGCCCTTGCAGCGTGG
ACCCCTTCATGCCTGTGGCGATGGCGCGGCATGGTGCTAAACGCCTTCGGAGCCGATATCCACAAGAGCGC
AATCGTTCGCGCAAGCGCACATATCTGGTGGCCGGGCAATCTGTCCATGGGCGCACATTCCTCGCTTGGCC
CCGGCGCGATCTGCTACAATGTTGCGCCGGTAACGCTCGCCCCCTTCGCCATTGTCTCGCAGCGCGCGCAT
CTTTGCACGGCAGGCCACGATATCGATCAGGCGAGCTTTCCCCTCACTGCCGCACCGATTCACATCGCTGC
CTATGGCTGGGTGGCGGCAGAGGCTTTTGTCGGCCCTGGCGTACATATTGGCGAGGGTGCAGTGCTGGGTG
CGCGCAGCGTCAGCTTCCGCGATCTGGATGCATGGGCCGTTTACACAGGCAATCCAGCAAAGCCCGTACGC
CAGCGGCGCAAAAACACAGATTATTCAATCCACCGGTTT
>BR0701
TTGCAGGATAAAACAGCTCTCATCGTCGGTGTAACCGGGCAGGATGGCGCCTATCTAAGCGAGCTTCTGCT
GGGCAAGGGCTATCGGGTGCATGGCCTGAAAAGGCGGTCATCGTCATTCAATACGGCGCGTATCGACCATC
TTTATCAAGACCCGCATGAGGAAGACATCCGCTTTCGCCTGCATTTCGGCGACCTGACAGACGCAACCAAT
CTGTGCCGCGTCATCCAGGAAGTGCAGCCTGACGAGATTTATAATCTCGGCGCCCAGAGCCATGTGCAGGT
GAGTTTTGAAACGCCGGAATATACGGCCAATGCCGATGCGCTCGGCACACTGCGCCTGCTTGAATCCATGC
GCATCCTGGGTCTCGGCAAAAGCTGCCGTTTCTATCAGGCTTCCACTTCGGAGCTTTTCGGCAATAGTTCC
CACCATGCGCAAAACGAGCAGACGCCTTTCGCTCCGCGCAGCCCATATGCAACCGCCAAGCTTTATGCTTA
CTGGACAACGGTGAACTATCGCGATGCCTATGGTTTCCACGCCTCGAACGGCATCTTGTTCAATCATGAAA
GCCCCTTGCGGGGCGAAACCTTCGTCACCCGCAAGATCACGCGCGCAGTTGCAGCTATCGAACGCGGGCTA
CAGGACAGGCTGCGGCTTGGCAATCTTGAGGCCCGCCGCGACTGGGGCCATGCGCGCGATTATGTCGAAGG
CATGTGGCGCATTCTACAGGAAGACACACCGGACGATTATGTGCTCGCCACGGGTGAAACGCATACGGTGC
GTGAATTCATCGAACACGCCTTCAAGGCCGTTGACAAGCAGATCGTATGGCATGGCGATGGCGTGGACGAG
GTCGGCATCGACCGCAAGACGGGCAATTGCCTGATCGAGATCGACCCGCGCTATTTCCGCCCCAACGAAGT
GGATTTCCTTATGGGCGATGCTTCCAAGGCAAAAGACCGTCTGGGCTGGCAACATACGACCGGCTTTGAAA
ATCTCGTGGCTGAAATGGTGGATTGGGATCTGCGCAACATCATGCGGGAGGTAGGACGCAATGATTTCTAC
GGC
>BR0680
ATGGCTGATCAGGCTATATCCGATACACGTCTTTCAGACGGTTCGCGCGGTTTTCTGATGGCGCTCGGAGC
TTATGGACTATGGGGCGCGTTGCCATTTTATCTCAAGGCGCTGTCTCACGTTCCGGCGATGGAAGTCGTCT
CGCATCGCGTCATCTGGTCCGTGCCGGTCGCGCTTGTGCTGTTGATCTTGCTTGGTCAGTTCAGGGGCATC
CTGAGTGCAGTTCGCCAGCCGCGAATGCTGGCCATGGCTGCATTGACGGCGGCGCTCGTCACTATGAACTG
GTCTGTTTATGTCTGGGCGGTTGCACATGACCAGGTGATGGGGGCCGCGCTCGGCTATTATATCAATCCCT
TGTTCAACGTGGTTCTGGGCCGTTTGTTTCTGGGCGAGCGGCTGACAAAAACACAATATGTCGCCGTGGGT
CTCGCCTTTGTGGCGGTCGTGCTTCTCACTATCAATGCGGGCGGCTTGCCCTGGGTTTCGCTGGCCCTGCC
GATCAGCTTCGGCCTTTATGGCTTCTTCCGCAAGTCGTTGCCGATGCCGCCCTTGCAGGGTTTTACGCTGG
AAGTGCTGATCCTTTGCATCCCCGCGCTTGCCTATGTGGCATGGCTCGCCGCAACGGGGCAGGACCATTTC
GTTGCGGGGCCCGTTTCCAATATTACGCTTCTGTTGCTGGCTGGACCCTTCACCGCCATTCCGCTCATTCT
TTATGCGGGCGGGGCGAAGCTTCTGCGCTATGCGACACTTGGCCTGATGCAGTATATGACGCCGACCATGC
TGTTTTTCTTCGCCATATTCATCTTCCATGAACCGTTCTCGACGGCGCAGCTTGCAGCATTCTTGCTCATA
TGGGCGGGACTGGCGCTCTACACATGGTCTTCGCTTTCCCAGCACAGGCGCAACCGCGCGCAGGCC
>BR0660
ATGAAGAGAATCCTTCTAGCTGAAGACGATAACGATATGCGCCGTTTCCTCGTAAAAGCGCTGGAAAAGGC
CGGTTATCACGTGACACACTTTGACAATGGTGCCAGCGCCTATGAACGGCTCCAGGAGGAACCATTTTCGC
TTTTGCTGACGGATATCGTCATGCCGGAAATGGATGGCATCGAACTGGCGCGCCGTGCGACGGAAATCGAT
CCCGATCTCAAGATCATGTTCATCACGGGCTTTGCCGCCGTTGCGCTGAACCCCGATTCGGACGCCCCCCG
CGATGCGAAGGTGCTTTCCAAGCCTTTCCACCTGCGCGATCTGGTGAACGAAATCGAAAAAATGCTGATCG
CCGCC
>BR0637
GTGTCTTCCTGTCGCGATTCGGACAGGAAACAGACAATGGCTGCAAGGAAACGAAGCTCCTCTTCGCGTTC
GCCAAAGCGCTCTTCCAGGCGCAAAAGCGGATCGGGTTCCTCGACGCCGATATTGGCGCTGGGCGCCATTC
TGGCTCTTGGCGCATTCAGCCTGTGGTCCACTGCGCAGCACAAAAGCCCACAAGCGGCTTTATCCGCGCTA
TTCCAGCGTCCCGCGCCCAAGCCCGCCCCGGCAGCTACAGCCAAGGCTGAAAACAAGCCCGCGCCCGGCAA
GACTGCCGATACGAAAGCGCCAGCCAGAGATTATGCGGCCGCCGTTGGCCCTGTTCCCCGCCCGGCAGCAC
CGGTTGCGCCCACACCGCAAAAGCCCGTCCAGACCGCGGCCGTCGCGGCGCCCAAACCGCCCCTCCCTTCC
GGGAGCGTCGTGGCATCGGTTACACCCGCTGCACCTTACGCCATGCCACCACGCGGCGTCAATACACCGGC
AAACTCGCCGTCGGTTATCTATGCCAAGGCCAAGCTCACCATTCACAAGAATGCCTGGGACCGTTCGCCAG
CCATTGCCACCATTGAGAAAGGCCGCGAAATGCGCTCCTACGGCAAGACAGGCCGCTGGCATCGTGTGGTT
GTACCCTCGACGAACATCATCGGCTGGGTTCACGAGGATCAATTGATCGGTGGACACAACAAGCCGGACAG
CGCAACGCTCATTACCGGATCAGTTGCCAAAAAGGCGCCATCGCCCGCGTCCACGCCCGCTCATGTGCAAT
CACACATCCTGCCGCCAAAGGCTGTCGGCGCTAAAAAC
>BR0618
ATGGCTGAAATATTTTCAGCCGGAGATTCGCACCGCTTACTTTTAAGGAATAGCGTCGTGAACGCCCATAC
GCGCGCAGCCGCCGATGCGGCCAGGCTTGAAATGAATCCGATGCTGGGCATCGGCCTCAAAATTGCTTCCG
TTGCTGTTTTCGTCGCCATGTCCACCTTGCTCAAGGCGGCGGAAGGCGTTCCGGTCGGCCAATTGATCTTC
TTCCGCTCCTTCTTCGCAATTTTCGCCATCCTGCTTTATCTGGGCTGGCGCGGGCAGCTTGGCGGGGTTTT
CTCTACCCGCCACGGCTTCAGCCATTTCTGGCGCGGGCTGGTGGGTGTGTGTTCCATGTCGATGAGCTTCT
TCGCGCTAACAAAGTTGCCTTTGCCGGAGGCCATCGCCATCAATTATGCCTCACCCCTGATTACCGTTATT
CTGGGGGCGGCCATCCTGCACGAAGTGGTGCGCTTCTACCGCTGGAGTGCGGTGCTGATCGGGCTGTTCGG
CGTCATGATTATCATCTGGCCACGCCTGACCCTGTTTTCTGCCGAAAGTGTCGGGCGCGACGAAGCGGTGG
GTGCGCTGGCCGCGCTTGGGGCCGCGGTCATGTCGGCTGTCGCCATGATGCTGGTGCGCCGTCTGGTCCAG
ACCGAACGTACGCCGACCATCGTGATCTATTTTTCCATATCGGCAAGCGTGATCGCGCTGGTGAGCCTGCC
GTTCGGCTGGGTTGTGCCGAACTGGTCACAGCTTGCCATGCTGGTGGGGGCGGGCTTTGCGGGCGGTATCG
GGCAAATTCTGCTCACGGAATGCTATCGCCATGCACCCATGTCTACGATTGCGCCGTTTGAATATACTTCA
ATGCTGCTGGGGCTGGTGATCGGCTTCATGCTCTTCGGCGATATACCGACCTTTGAAATGTTGATCGGCAG
CGGCATCGTGATGGCTGCCGGCGGCTTTATCATCTACCGCGAGCACCAGCTTTCGTTGGCGCCGCACAAGG
TTAATGCCCCGCAGGCGCAA
>BR0615
ATGAAAATCTGTACCTCTATTCTCGCTCCTTTCATACTGGCCGTGCTGGCAATCCCTGCACAGGCTTCAAC
TTCCGCCTGGACCGAAACTCCGGGCGGGCGTGTGCGTATTGTTATCGAAGGTGGCGATCCGGCAATCACCG
GAAAACAGGAGGTGCGCGGGGCCTTGCAGATCGAACTTCGCCCCGGCTGGAAAACCTATTGGCGCAATCCC
GGCGATGCCGGTGTGCCGCCGCAGATCAGCATTGAGGGCGATGCGAAGGCGCAAATTGCGTTCCCCGCGCC
TGTACGCTTCGCCGGGGACGAGGAGGGCGGCATCGGCTACAAGAAGCCGGTTTCGCTGCCCGTCACCTTCC
ACATCGAGCCGGGGGGCAAGCGGCTGAAAGGTCATGCCTTCCTCGGCATGTGTGAAAATATCTGCGTGCCG
GTGCTGGCCGAATTCGATTTTCCGCTGGATGCCGACAGGCAAAAATCATCATCGGAGAATCTCGCCGCACG
CACCGTCGTCGAGACCGCCTTCGCACAACTGCCGGCACCTGCTTCACCCGATTTCGGTGTGAGCGCGGTCA
AGCACGACAAGGATGAAGCCATCTTCGACGTCGCCCTGCCCGATCCCAATGCGCCTGCCGAGCTTTTCGTG
GCAAGCGACACGGTCAATCTTTCCGCGCCGGTTTCCGTCGAGGATGGAAAAGCCAGCCAGCGTTTTATCGC
CAAGATGCAGGGCAAGACGAAAGATGCCGTTATCGACTATACGATCGTGCAGAATGGCAAGGCAGTTTCCG
GCCGGCTGGAACTGGAC
>BR0613
ATGAGCCGGTTTTCCGCACTCATGCGCACCACCGCGGCGCGGCTTTCCGCGCTTTATCTCCTGCTCTTTGC
AGTGGGCGCGGTTGCACTTGTCTTTTACATGACCAACCTTTCGGCTTCCATTCTGGCCGGGCAGACCCAAC
AGGCGCTGGGCGAGGAAGTCGCGAGCATCGGCAAAAGCTATGCGCGCGGCGGTATTCCGCAACTTGTGCGA
ACCATTGATTATCGCTCGCGCCAGCCGGGTGCCTATCTCTATCTTGTCGCCGACCCTACGGGCCGTATTCT
TGCCGGTAATGTAGAAAGCGTTGAACCGGGCGTGCTCAACACGGACGGTATTATCGAACGTGCCTTTACCT
ATCGCCGTTACGGCGAACAGGCGCCGCAGATGGAGCATCGCGCCATTGCCGTGGTGATTGCCCTGCCGAAT
GGAATGCGGCTGCTCGTCGGGCGGGATCTGGGCGAACCTGAGCGAT
TTCGCGATCTGATACGCAACTCGCTGGTGCTGGCGCTTGGCATCATGGGCGTGGGTGCGCTTTTGATCTGG
CTCTTCGTGGGGCGGCGCGCGCTCAAACGCATCGACGATGTGTCGCGCGCATCGCAACGCATCATGGATGG
CGATCTGACCGGCAGGCTTCCCGTCAACGGCTCGGGCGACGAATTCGACCGGCTGTCTGGCAATCTCAATG
TCATGCTGGCACGCATTCTGGAATTGAACGAGGGGCTGAAACAGGTCTCCGACAATATTGCGCATGACCTG
AAAACGCCGCTCACGCGGTTGCGAAACCGCGCTGAGGAGGCGCTTGGCGGTGAGAAGGTAGAGCCGGAATA
TCGCGCTGCGCTGGAAGATATTATCGGCGAATCCGATCAGCTTATTCGCACGTTCAATGCCATTTTGATGA
TCTCGCGGCTTGAAGCCGGTTATTCATCTGAAAACCTCGACGATATGCCGGTTGCCCCGATCATGCGCGAT
GTTGCGGAAATGTATGAGCCGGTGGCAGAGGATGCGGGTGTTACCTTGACGCTTGGTGCGCTTGATGACGT
AGCCCTGCACATCAACCGCGAACTGGTTGGCCAGACCGTGTCCAATCTGGTCGACAATGCGATCAAATATG
CGGGCGGCGAAGGACGCACGGCGACGGTCACGCTTTTGATGGAAAAGGATGCGCAATGGGTCCATATCGTC
GTGGCTGATAATGGCCCCGGCATCCCCGCCGACAAACGTGACCATGCGACCGAGCGTTTCGTGCGCCTTGA
AGAAAGCCGCACACAGCCGGGGTCTGGCCTGGGTTTAAGTCTTGCCAAGGCGGTCATGAAGTTGCACGGTG
GCGCGCTTCGTCTTGAGGACAACGGACCCGGCCTTCGCGCCGTTCTGGAATTTCCGCTGCCGCATCGCGAG
GTTGGC
>BR0611
ATGGAAAAAAGCCGTAAATCGCATGACAGGTCCGTCCTGCGTTTTTTGCGTCATCTACTGGAAAACGAGTA
CGCCATAATGACCCAACAGCGCCCCCCTCTGGAGATCCGCCTTTGCGGCCCGCGCGGCTTTTGCGCAGGTG
TGGACCGGGCCATCCAGATTGTCGTCCTGGCGCTCAAAAAATATGGTGCGCCGGTTTATGTGCGCCATGAG
ATCGTGCACAATCGTTATGTGGTCGAGGGGCTTCAGGCCCGCGGCGCGATCTTCGTCGAGGAACTGGATGA
AATCCCGGCGGCACACCGCAACCAGCCGGTGGTGTTTTCCGCCCATGGTGTGCCAAAATCCGTGCCAGCGG
ACGCGGAGGCCAAGAACCTCTTTTATCTTGATGCGACATGCCCACTGGTTTCCAAGGTGCACAAACAGGCC
ATGCGCCACCAGCGCCTTGGCCGCCATGTCATCCTGATCGGCCATTCCGGCCACCCGGAAGTGATCGGCAC
GATGGGGCAATTGCCGGATGGCGCGGTCACGCTGATTGAAACCGTCGAGGATGCGCACACCTGCCATTTCG
ATGATGAAGACAATCTTGGCTTCGTCACGCAGACCACGCTGTCGGTGGATGACACGGCGGGTATCATCAAG
GAATTGCAGGCGCGCTTTCCCAATCTGGCGGCTCCGGCGGCGGAATCGATCTGCTACGCCACCACCAACCG
GCAGGATGCTGTGCGTGCGGCGGCGCCGGGCTGCGATCTGTTTCTGATTGTCGGTGCACCGAATTCCTCCA
ATTCCAAGCGTCTGGTCGAGGTGGCGGAAAAGGCGGGCGCGCGCATGTCCATGCTGGTGCAGCGCGCGGAG
GATATAGAATGGGAGCAGATCGGCGATATTTCCGTCGTTGGCCTTTCGGCTGGCGCGTCTGCGCCCGAAAT
CATCGTGGATGAAATCATCGATGCCTTCAAAGCCCACTTCGATGTGAAGATCGAACTGGCGGAAACAACGG
TTGAAACCGAAAATTTCCTTGTGAATCGCGAAATCCGCGATGTGGAATTGACGGTGAAGGATATGGCATTC
GTCAATGGCGAGCACCGCGTGGTCGGCATTTCCAAACTTATGCAGGGGAAG
>BR0605
ATGACGGATCAGGGGGAGAACGAGAAGAAGCAGCGGCGCTCCAATGCGACGATCGCCGTCGCGTGCCTGTC
CTTCTTCGTCTGCATGATCGGTGCGGCCTATGCTTCCGTGCCGCTCTACCGCATCTTCTGTCAGGTGACAG
GCTATGGCGGCACCACGCAGCGTGTGGAGCAATATTCCGACACCATTCTCGACAAGACGATCAAAGTGCGT
TTCGATGCCAACATCGCAAATGGCCTACCATGGGATTTCAAGCCCATGCAGCGCGAGGTGACGGTGCGTAT
CGGCGAAACCACGATGATAAAATATGAGGCGCATAATCTGTTCGGCGAGGAAACCTATGGCCGCGCAAGCT
TCAATGTCGCGCCGGGCCGGGCCGGGGCCTATTTCAACAAGGTCGAGTGCTTCTGCTTTACCGATAACACG
TTGAAGCCGGGTGAAGACCTTGAACTGCCGGTCGTTTTCTTCGTGGACCCGGAATTCGTGAATGACCCGGA
CCTGAAGGATGTGAAGACGATCACGCTGTCCTATACTTTCTTCCCGATTGATAAGCCGAGGCCGGTGGTGA
ACGCGAAGGCAGTGGGCAGCACCCGCAACGGCGGC
>BR0583
ATGCGTAATACGGCAGAAAAAGATCGCAGAAACGACAAGCGTAAACCCTTCCGGGTTTCGCGCCTGATCGG
ACTCGACGCCTGGATTGATTCCGCGCTCTACAATTTACATTTCCGCTTGGGCGAATGGTGGGAAAACATCA
CGATTTTCTCCCGGCGTTTTCGCGTGCGCGGCTTTCGCCGTTTCACGGTGGAAGTGCTGGACGAAGGCTTT
ACGCTCGGCGTCGCCGGTTCCGTGCTGATGTTGATGCTGGCGCTGCCAGCCTTTGAGGAAACCAAGAAGGA
CTGGCGCGCGCAGGACGATTATGCCGTCACCTTTCTTGACCGTTATGGCAACGAGATTGGCCGGCGCGGTA
TTCTGCACCGGCAGGCGGTGCCTATCGATGAATTGCCGGATCATGTCATCAAGGCTGTGCTCGGCACCGAG
GACCGTCGCTTCTTCGACCATTACGGCATTGATCTCATGGGGCTTAGCCGAGCGTTAAGCCAGAACATGCG
CGCCAACGGCGTGGTTCAGGGCGGCTCGACCATCACGCAGCAGCTTGCCAAAAACCTGTTTCTTTCCAATG
AGCGCACCATCGAACGCAAGGTCAAGGAAGCCTTTCTCGCGCTCTGGCTTGAAAGCAATCTCAGCAAGAAG
AAAATCCTCCAGCTTTACCTCGACCGTGCCTATATGGGCGGCGGCACTTTCGGCATTGCAGCCGCGTCCGA
GTTTTATTTCGGCAAGAATGTAAAGGACATCTCGCTGGCCGAGGCAGCCATGCTGGCCGGCCTGTTCAAGG
CTCCGGCGAAATTCGCGCCGCACGTCAACCTGCCTGCAGCTCGCGCCCGCGCCAATGTCGTTCTCTCCAAC
ATGGTGGAAAGCGGCTTTCTCAGCGAAGGTCAGGTTGCCGTCGCGCGCCGCCACCCGGCCAGCGTCATAGA
TCGCGCCAAGGATGAAAGCCCCGACTATTTCCTGGACTGGGCCTTCGACGAGGTGAAAAAGGTTGCAGACA
GGTTCAACCAGCATACGCTGATCGTGCGCACCACGCTTGACCGCAATATCCAGAAAGCCGCCGAAGAATCG
CTTGAATTTCACCTTCGCCAATATGGCAAGGAATATAACGTATCGGAGGCGGCCACCGTCGTGCTTGCCAA
TGACGGTTCCGTGCGCGCGCTCGTTGGCGGGCGCGACTATGGCGAAAGCCAGTTCAACCGCGCAACCAGGG
CGCTGCGGCAGGCCGGTTCATCCTTCAAGCCCTATGTCTATGCGGCTGCCATGGAAAAGGGGCTGACACCA
AGCACCATCGTTTCCGATGCACCGATCAGCTGGGGCAACTGGTCGCCGCGCAATTACGGGCGCAGTTTTGC
TGGCCGGGTTGATCTCACCACCGCGCTCGTCCGCTCGCTCAACAGCGTGCCGGTGCGCCTTGCCCGCGATT
ATCTGACCACGGCGCCCGTTGTCGCGCTCACAAAGGCAATGGGTGTGGAATCGCCCATCTCCTCCCATAAG
ACGATGGTGCTCGGCACATCCGAAATGACCGTCATGGATCAGGCAACAGGCTTTAACGTCTTCGCCAATGC
GGGCATGGCCGGAAACCGTCACGCTTTCACGCAAATCCTGGCATCCGACGGAAAGGTTCTTTGGGATTTTG
GCCGTGACGCACCAAAGCCGCACCGCGCCCTTTCGGAAAAGGCCGCACTTGAGATGAACTCCATGCTGGTG
CAGGTGCCCGAGCGCGGCACGGGCCGGCGCGCCGCACTTACGATGACGCGCGTGGCAGGCAAAACGGGTAC
GACCCAGAACTATCGCGATGCCTGGTTCGTGGGCTTTACCGGAAATTTCACCGCCGCCGTCTGGTTCGGCA
ACGATAATTTCACGCCCATGAAGGAACTGACCGGCGGTGTTCTGCCAGCCATGGCCTGGCAGCGCATGATG
GCATACGCCCATCAGAATATCGAATTGAAGCCGCTGCCGGGCGTCAACCCGCCCTTCCCCGCCCAGCCGAA
GAACCCGCCCGCACAGGTGGCCGATACGAGGCCGCACGAAACAATGGCTGCCCCACCACGGGTTTTATCGC
CGCTGGCGACAAAAATTCTCAAGGAACTGCATGACCGCTTCCTTGCCGCGCCGCCTCTGCCTACGATAGCC
GAGCGCACAAAAGTTTCGGTACTC
>BR0544
TTGCCCTATCGCACAGGCTTCGCTATCCTTCAAGCAATTGCGGATAAAAAGGCTGGTATTGTCACGTTACT
GGCGCAAAGTGACAACACCTTCGCTTCAACCGAT
>BR0541
ATGCGCCTGCACCGGCAACGCACGCATGGCCACATGTCCTGCGCACCTGCTTCATTCGATAATCCAGCTGC
ACGCGAACTCCTCCCAAAGCTCTACGTCAGGAAGCTTATCTTTCGCGTTGAGGTACGTAAAGCAAAAATAT
TAGGGCGGACGCAAAGAGTAGCCCGCCTGAAAATCGCACCATTTCACAAAATTTATGGCGGGATTTTT
>BR0540
ATGCGAGTTCTTCATTTTTTTAAGACCTATTGGCCAGATACATTTGGCGGCATTGAGCGAACCATTCATGC
GATCGCAAAAGGTGTGGCCGAACACGGTATCGCGTCGGACGTGTTATCCTTGAGTGCAAAGCCGGAAGAAA
ATACCCGAAATTTTGATGGTCACATGGCATATAAGGCCAAGCTCGACCTTGAATTTGCATCAACAGGATTG
TCGCGCGATGTGTTTTCCCGGTTTCGGGAGTTGAGCAGCCAAGCTGATGTCATTCATTATCATTTCCCCTG
GCCCATGAGCGATATCGTGCAGATCGGTGTGCGCCCGGATAAGCCGACTATCGTGACCTATCATTCGGATA
TCGTTAAGCAGAAACTACTTCTTCAGTTCTATCGCCCCTTGATGAACCGCTTTCTCGGCAGCGTTGACCGG
ATCGTTGCCACCTCTCCGAATTATCTTGCGACAAGCGATGTCCTGCAGCGGTTCAGCGATAAAACGACAGT
CATTCCACTTGGCTTGGACGAGTCTGATTATTCTGCTGCCGACGCCACAACCAAGGCGCGCTGGCGCGAGC
GGTTTCCGCGCCCTTTCTTTCTCTTCGTCGGCGTGCTGCGTTATTATAAAGGGCTAAGAACGCTACTGGCC
GCAGCAAGAACTTCCGATATTGATATTGTCATTGTCGGGGATGGGCCTATGAAACCTCAGCTCATCGCGTA
TGCGAATGAGCATAATCTGTCGCACGTTCATTTCGCCGGCGCGCTGCCGGATACGGACAAAACTGCGCTTC
TGGAGTTGAGTGCTGGGCTGATTTTTCCGTCGCATCTGCGCTCGGAAGCGTTTGGGCTATCGCTGGTGGAG
GCCTCTATGTTTGGAAAGCCGATGATCTCGTGTGAGATCGGTACCGGCACGAGTTATGTCAATCTGAATGG
GCAAACGGGAATCGTGGTGCCGCCTCAGAACCCGGAAGCGCTCTCAGCTGCAATGCGAAGCATTGCACACG
ATACGGAGCAGGCCAAAGTTTTTGGAATGAACGCACGGGCGCGATATATAGAGCATTTCACTGCTGATAAA
ATGGCGTTAGAATATGCGAAAAATTATACGTCGCTGATGCAG
>BRA1122
ATGAAAAATCTGCATTCCTTGTTTCTTGCTTCTGCCTTTCTGGCTGGCTTTTGCGGTTCTTCCCTTGCAGG
CGAACGTGAGGGCGTGGTTGTTTCGATCAAGCCGCTTCACTCGATTGTTTCCGCCGTCATGCAGGGCGTGG
GTAAGCCGAAGCTGATCGTTCAGGGGGCTGGGTCCGAGCATGTTTATAGTCTGAAACCCTCCGATGCCGAG
GCTATCGAGCACGCCAAGGTGATTTTCTGGGCCGGGCCATCCATGGAAACCTTTCTCGACAAGCCCATTGA
TACGCTGGGCGAGGGCGCGAAAGTGGTGGCTCTTGGCGATGCGAAAGGGTTGACGAAACTAAAATTCCGCG
AGGGTGGGCCGTTCGAGGCGCACGATCATGGCCATGGGGGGAGCCATGAGGAAGAGCATGACGCACATGGT
TCAGGCGATCACGACCATGCGGCCGAAGTCGCAGAAGAAGGCCATGAACACCACCATCACGGCGAATATGA
TCTGCATTTCTGGCTTGACCCGCAAAATGGCAAGATTCTGGCTGCCGATATCGCCAAAACGCTTGGCGAGA
GCGACCCGGAACACGCCGCGCAATATGAAAAGAATGCCAAGGCCTATGGAGAAAAACTCGATGCCTTGACC
AGGGAAGTGGCTGCTGAACTGAAGCCGGTCAAGGACAAGCCCTTCATCGTCTTTCATGATGCCTATCAATA
TTTCGAGAATCGCTTCGGCATGAAAGCAGCCGGCTCCATCACGGTCAGCCCGGAAAAAGCGCCGGGTGCGG
CGCGTATCCAGCAAATTCACGACAAGATAAAGTCACTCGGTGCAACCTGCGTTTTCTCAGAGCCGCAATTT
GAGCCGAAACTGGTGAAGACGGTGGTTGACGGAACGAAAGCCCGGACAGGTGTTCTTGATCCTCTTGGCGC
AGAACTGAAGGACGGCCCTGATCTTTATCCGCAACTCATCCGCAATCTGGCCAATTCGCTGAAAGACTGCC
TGTCGAAA
>BRA1144
ATGTTTCGATGGGGTGTACTTTCCACCGCGAAAATCGGCGTCACACAGGTTATTCCAGCACTTGCTGCTTC
GGATAACGGTGTGGTCCATGCAATAGCAAGCCGCGACCATGCGCGCGCACGCGCGGTTGCCGGCCGCTTCG
GCGCGCCGCTCGCTTTCGGTTCCTATGAGGAATTGCTGGCCAGCAAGGAAGTGGACGGTGTCTATATTCCG
CTGCCGACATCCCAGCATATCGAATGGACATTGAAGGCCGCCGAAGCAGGCAAGCATGTGCTGTGCGAAAA
gCCCATTGCGCTTTGCGCGGGCGATATAGACCAGCTCATCGAAGCGCGCGACAGGCATGGCGTCACGATTG
CGGAAGCCTTCATGGTCTATTACCACCCGCAATGGATCAAGCTGCGCGCGCTTCTGGCCGAAGGCGCGATT
GGCAATCTGCGTCATGTGCAGGGTGTGTTTTCCTATTTCAATGTCGATCCCGGCAATATGCGCAACCAGCC
GGGCCTTGGCGGAGGGGCCTTGCCGGATATCGGTGTTTACCCAACCGTGGTGACGCGCATGGTGACAGGCA
GGGAGCCGCTCTCGGTGCGCGCTTCGGTGGAATATGATCCGGGTTTTGGAACCGACCGCTACGCCAATGTT
TCGGCCCGTTTCGACGGGTTCGACCTGACCTTCTATGTCGCGACGCAGCTTGCCGGCCGCCAATGCATGGT
TTTCCATGGCGATAAGGGCTTCATCGAGGTTCATGCGCCATTCAATACCGGCAAATATGGCCATGGCCGCA
TCACCCTGCACGATGCGAGCCATATGCAGGCCACCGAATGGTCGTTTGGCGATGCAAACCACTATCAGCTT
CAGGCGCAGAGCTTTGTTCGTGCGGCGCGGGGTGAAAATGTGCCGCTCTTCTCGCTGGAAAATTCCAGGGC
GAACCAGAAATTCATCGATGCGATTTATTCCGCGGGCAGAAGCGGGGTGAGCGAGACTGTG
>BRA1190
ATGAAGTTCACGAGAACGCTGGTGCTCGCCAGCACATTTCTGCTTGCCACCGTCGCCACATCGCAGGCTCA
GGAAGTAAAGCGTGATACGAAAAAGCGGGGTGAAGTTGTCCTTAAACCCATTACGATCATAAGCCACGGCA
AGGACAATATCGAAGCGACCGGCGGCACTGTCCTTACTTATAAAGATATTGAAAAGCTGCAACCTGCCAAC
GTTTCCGAGTTGTTTTCGCGCCAGTCCTCCATCGCTGTTTCCGGCGGTGGCGGCCCGTCGAAGCGTATTCA
CATTCTTGGCATGGAGCAATCCAATCTGGCGGTCAGTGTGGATGGCGTGCCGCAAACGGCGACAAGCTGGC
ACCATACGGGTTCCAACGTAATCGATCCTGCGTTTCTCAAGCGCGTGGAAGTGGAGGCCGGAGCGGCCGCC
GCAGATTCCGGCTTTGGCGCGGCCGCTGGTGCGATCCGCTACGAGACCGTTAATGCGCTCGATCTGCTGGA
GCCGGGCAAAACCTTCGGTGCGCGCATCATAGGTTCCTACGGTACAAATGGCAGGGGTTTTTCCGGCAGTA
CCGCCGCCTATGGCCTCAAGGATGGCTTCGACTGGCTGCTGATGCTGCATGGCACGTCGGGCCACAATTAT
AAAAACGGCGATGGAACCGAAATACTCGGCACCGAGCCTGCCGCGCGCAATATTCTGGGCAAGGCCGGTTA
TGAATTCGACGGCAACCGCATCGATATTGGCTATGAACGCAGCCGCGACAAGGCCGACCGTCTTATCAAGA
TGAATATGGGGCTGCCCGGCGACACCGAATATCCGCTTGAGGTTGCGCGTGATTCCGTGAACATAAAATAT
ACCCGTACCGATGCTACGGACATGTGGGATCCGGAGGTGCAGTTCTATTACAATCGCAACGATTATTGGCG
CAACGATTATCAGAACAGGACAAACGGCAACATGATCCTCAAGGAGGATCTGTATGGCGGCAAGTTGCAGA
ATACTTTCACGATCGACTACGGCAAGATTACCGCCGGTATCGATTTTGGCAAGCACGACTACAATACGGAC
AATTACGGTCATAATGACCGCCGCTACCGCAAGTTCAACACCCAGCAGGTCGGTGCATTCACGCAAGGCCG
GTTTGAATTCGATAACGGCTTCAGCCTTTCGACAGGTGCCCGCTATGACTATAGCCGGTTTGCGGACTGGA
ACGATGAGGTGTTTTCAGATAGCGGCGCCAGCGTGAACGGCACGCTATCATACAAGTTCAACGAGCATATC
GAGGTTTTTGCCGGTGCTTCACGGACCTGGCTTGGCTACGTCCTCGGTGATTACGGCTATGTCCACGCCCG
CAATAATGCTTTCTATACCGACCCGACCTTCAGCCCCGGAAGGGCGCGCAATTATAAAGCGGGTGTGAATT
TCGGCGGTGCAGACTGGAGTGCCGGCATTACGCTTTTCGACACGCGCATCGCCGGTCTGCCGAATTATGAC
AGCCAGAAGCTTGGCAACGATCCGGAAGAATATCGTTCGCGAGGCTTTACCCTCAATGCGCGCTATATCTG
GAACTACACGACCATTGGCGCAACCTTTACCAAGGCCAAGGTTACGGCGGGCGACGACCCAGTCTTGCCGA
ACAGCGGCAGCTTCATGCCGATCGGTGATATGGCGACGCTTTTCATCGACCAGGAAATCCCCGACTACAAT
ATGAAAGTGGGTGCTACGCTGGCCTGGGCGGGTAGAATTTCAGACGAGGCTGCTACTGCTGCCAACTTCTA
CGATCAGCCTGCCTATACGGTTGTCAACGCCTATGCGGAATGGAATCCGCCAGCGGTTAAGAATATGACGT
TGCGCGTGGGTGTCGAGAACCTGTTCAACGAAAACTATTACGAGAGAACCAGCTTCGCGCCGAGTCAGAAG
CGTGGTGGCATCGATCCGGTTTGGGCGCCGGGCCGGACCTTCACTTTCCAGACGGCCTTCAAGTTC
>BRA1202
ATGAGCGAGATCGGTTCTTATCACGATACGATTTTTGCATTATCGAGCGGACGCTTGCCCTCAGGGGTCGC
GGTCATTCGCATTTCCGGCCCAAAAACTCGATTCGTTTACGAAACAATCTGCCAAGCAATTCCGGAACCGC
GCCATGCCGCATTGCTTACATTCCGTAGCCGAAATGGTGACGCCATTGATCGCGGCCTCACTCTGTTTTTC
CCTGCTCCTCATAGTTTTACTGGGGAAGACTGCGCCGAATTTCATTTGCACGGCGGTAAGGCCGTTGTGGA
AAAGATGCTTGCGGTGTTGGGCGAACTGCCGGGATGCCGAATTGCGGAGGCTGGTGAATTTACGCGACGTG
CTTTTGCCAATGGCAAGATGGACTTGACGATTGCAGAAGGTCTGGCCGACCTCATTGCTGCGGAAACCGAG
GGGCAGCGACGGCTTGCAATGCAAGTGGCTTCCGGTAATCAGCGCAAACTATATAGTGAGTGGCGCCAGCG
ACTTATCAATGCGCGGGCTTTCATTGAAGCTGAACTGGATTTTGCCGATGAAAGCGACGTACCCGGTTCTG
TTTCCATGCAGGTCTGGCAACAGCTTTCGGCGCTCAAACATGAAATCGAGCACCATATCGCAAGCGGTAAG
CGCGCTGCGATGCTGCGGGATGGATTGCATGTTGTTATTGTCGGCGCGCCAAATGCCGGTAAATCCAGCTT
GCTTAATTTTCTAGCCGGGCGCGATGTTGCGATCATTTCCAAAGAAGCTGGTACGACGCGCGATCTTCTGG
AAGTGAAACTCGATCTCGGCGGTATCCCGGTTTATGTCACCGATACGGCGGGTCTTCGCGAAACGGATAGT
GTCGTTGAGAAGATCGGCATTGAGCGTGCGCGTGCCCGGATGGCCGAAGCCGATCTGGTGCTCTCGCTGGA
AGATATGAGCGGACCTGTATCTGTTACCGTTGAGAAAATCGAAGCAGAAACCTGGCTGATCGGTACCAAGG
CCGACCTCGGTGGAAGTGCTTCGGGCTTGTGGAAATATCACATTTCCACCATGACTGGCAGTGGTCTGGAA
CAATTGCTGGACGCTCTACAGGCTTTTGCTGAAGCGAAGATCGGCCAGATTGAAGATGCTGTTCCTACCCG
GCAGCGCCATATCAACCTGTTGCGGGCAACGATTGAAGAAATCGAAAAAGCAATTGAGGGTGACGATCTTC
CATTGGAGTTAAGAGCCGAGAATATGCGGCTTGCTTCGCAATTCCTTGGACGGATCACGGGCGATGTGGAT
GTAGAGGAAATTCTGGACGTTATTTTCTCGCAATTCTGCATCGGGAAG
>BRA0004
AtGAACAAGATGTCCAGGATCATAACCATTGCGAACCAGAaGGGTGGGGTGGGCAAGACCACTACCGCCAT
TAATCTGGCGACCGCGCTTGCTGCCATCGGCGAGACGGTGCTGATTGTCGATCTTGATCCGCAGGGCAATG
CCAGCACGGGTCTCGGCATTGACCGGCATAACCGCCCTTTGTCCTCCTATGATGTGCTGACGCAGGCTTCG
TCCGTACCGGAAGCTGCGATGCAGACGGATGTGCCCAACCTCTTTATTGTGCCATCAACGCTCGATCTTCT
GGGCATTGAAATGGAAATCGCCCAATCGGCAGATCGTACCCGGCGTTTGCGCGATGCATTGCGTTTCGATT
CTGGTGTTTCGGAGCGATTTACTTACGTTCTGGTCGATTGCCCGCCGTCCCTTAATCTGCTGACATTGAAT
GCAATGGCCGCTGCCGACTCCGTTCTGGTGCCGCTGCAATGCGAATTTTTCGCGCTTGAGGGGTTGAGCCA
GCTCTTGCAAACTGTCGATCAGGTGCGTTCCACCATCAATCCTGAACTGTCCATACAGGGGATTGTGTTGA
CAATGTTTGACAGCCGCAACAATCTGGCAACGCAAGTGGTAGATGATGTGCGTGCTTTCATGGGCGAAAAG
GTCTATCGCACGGTTATTCCGCGCAATGTTCGTGTTTCGGAAGCACCGTCCCATGGAAAACCAGCTATTCT
CTACGATCTGAAATGCGCCGGTAGCCAGGCCTATCTGCAACTCGCCTCGGAGGTAATCCAGCGCGAGCGGC
AATTACAGGCTGCC
>BRA0018
GTGGCCACCGGCAAGGTTTATGAACAGCAGGATGCCTTCCAGCGCTGGTATCCCGCTTCGCTCACCAAGAT
GATGACGGCCTATGTCACATTCCGCGCGCTTCAGTCCGGCCAGATGACGCTGGAATCCCCGGTGCGCATGA
CGGTCAACGCCTCCAAGGAACCGCCGAGCAAGATGGGGTACAAGCCCGGCTCGATCATGACGCTTGATACG
GCGCTGAACATCATGCTGGTCAAGTCTGCCAATGATGTTTCGGTGGCCGTTGCCGAAGCCGTGGGCGGAAC
GGAAGCTGCCTTCGTGCAGCGGATGAATGCCGAGGCGCGGCGTCTTGGAATGTTCGGCTCGCATTTCGCCA
ATCCGAATGGCCTGCATGATCCGAACAATTATACGACAGCGCGTGATCTTGCGGTTCTTGCCGTGCAGTTG
CGCCGCGAATTTCCGCAATATGCGCATTATTTCGCGACAGAAGCGATCGATCCGGGTGCGGGCAAGAAAGT
TGAGGCCAATTACAACATCCTGCTTGGCCGCTATGACGGTGCGGATGGCATGAAGACGGGCTTCGTCTGCG
CATCAGGCTTCAACCTTGCAGGTTCGGCCACCCGCAACGGCCGCACGGTGCTGGCAATCGTACT
GGGTGCGGATCGCCAGGAAACGCGTGCGATCCAGGCCGCGCAAATGATGACCGATGCGTTTCGCGCGCGCA
GTTCCGGCGGGCCGACGCTTGCCACGCTGCGCCCGGCGCAGACAGCCAATCTCAATCAGGCCGTTGATATG
CGCAAGGCCATCTGTAGCGAGCAGGCCATGGCCGACCGCTGGGACGGGCGCGAGGTAGAAGGCCGGCTGAA
GATCAATTCACCCTATATTCACGCCATGGACCATGATCCGATTGCCGTGCAGGTGAGGCTGGTTTCCGAAC
CGGGTTCGATCAACCGGCCCGGCCAGCCCGGCATTTCGCGGGTTCCGGTGCCAATGCCGCGTCCGCAGCGC
GCGCCAGGCGTAAAGACAACGGCCATCAAC
>BRA0020
ATGACCATCAAGCACAACATCATCGCCTTTTCCGCCCTCGCCCTGATTGCAGGTTTCTCGCTTACGCCAGC
CGCTTTCGCCGAAGATGCGATGAAAACCGACCATATGAAGCCGGACGCGATGAAGAGCGATAATATGTCAA
AGGATGCCATGGGCAAGGAAGATGCCATGCACAAGGATTCCAAGAAAATGAAGAAGCACGACGCCATGAAA
GGTGACGCAATGAAGAGCGGTCAG
>BRA0037
ATGACAGATGTCTTGCTCCTGCTGAAAGCCGCATGGAAGCGATCGATCCGCTATGGTGCATTCACCCTGGC
AGGACTGGCAATCGCGAGCGGAGTGGCGAGAGCAGATGATCTCTATAGAACCGAATATGATATCTCGATTT
TCGGGCTTTCCATTGCGCGCGCAGCTATCGAAACCGTCGTCAGTGGGCCGAACTATAACCTGAACGGCCGT
TTCGTGACATCGGGCCTTGCCCGCATTTTCGACAATACGGATGGCACCGTGCACGTCACAGGCAGTGCGGT
AAAAGGGGCTATCGTCCCCAAAAGCTTCGATCTCGCCTATAAACATGGCCGCAAGGACAAGAGCACGGCAA
TCCGTTTCATGAATGGAGACGTGGTTGCAGCACAAAACCAGCCACCCGTGAAAAAAGACGATCCCTGGGTG
GAAACCATGCCAGGCGATCTCCTCAATGTCAGCGATCCGCTGAGCGCCCTCATGATCCCCGCAAAGGATGC
GCGTTCGGTCTGCGACCGCTCGCTCAGTGTTTTTGACGGCCAGACGCGCGTGGAAATAAAGCTGGCCTTCA
ACGGCACTGAATCGTTCACCACGGGTGGCTTTACTGGCGAATCAGTCATCTGTTCGGCGAAGTTCATTCCG
GTTTCAGGTTATCAGAAGGGCAAGAAATCGATCGATTATCTGGCCAACAAGAGCCGGATCACCATCTCCTT
TGCTGCGCTCGGCAATTCCGGCATCTATGCGCCGGTGGTGGCGCGTATCGGCACCCGGATCGGAACGCTGA
AAATCGCAGCCACCCGGTTTGAAAAAGTGGAA
>BRA0059
ATGAGTTTTTTTCCCGCACGAACCGTCAAAAAGCTGCAAATATCCGGTAATTTTTCGAGTTATCGCCGCAG
TGCGGGTTATGATAGCAAGATAAGGGAAACACAAGTTACGGAAGCGGCATCGGGCGGGTATTGGACAGGTG
ACGATGCAGTGGCACAAAGACGGACAGGAAGCAGTTCGCAAGCAGATATATGGCGCCGCAGCCATCAGGAT
GCATTTGTACCTTCGTTCCTGGGCACACCGGCCAGATTCGCCTGGTCTGTCATGATGCGGATATTGCGGCG
CCTCCAACCTGTTTCGATCATCCTTTTCCTGGCAGTCATAGCCTTTCTGGGTGGCTTCTTGGTGTTCAGTG
AAAAAGTGACGGGAATGCAGCCGCCCGTTTTGAGCGAACCGGCGGAAGCAATTGTGGTTTTGACCGGCGGG
CAATCGCGCGTCAAGGCAGGGATCAACCTCTTGAAGGAAAAGCGCGGCAAGCGCCTCCTTATCAGCGGTGT
GCATCCATCCACTAATGAGGAGGCGCTGCAAAGGGCCACCCATGCGGACAAGAGCCTGTTTGCCTGCTGTA
TCGACCTCGACCGTACGGCGCTCAATACGGTGGGCAATGCGGCGGAAAGCGAGCGCTGGATAAGGGCAAAC
GATTACCACCGCGTCATCGTCGTCACCAATAATTACCATATACCGCGCAGCATTCTGGAAATGTCGTCGCG
CATGAAGGATGTGGAGTTCATTCCCTATCCGGTGGTGAATGGGGAAAAGCGCGAGCAGAGCTGGGTTGCCG
AAGGTGATACGCTGCGTGTGCTCTTCACCGAATATGTCAAATATCTGGGCGCCGTTGTTCGGCTTGGCAGC
GCCTCGTTTTATGGCAATGGCGCTTCTCATGGCGGCGATATGCGCGAA
>BRA0076
GTGGAGATAGGTCTAACCCAAATTGCCCGAAAGTGGAATCATCTCTTGGCGGGTGATTCCACATGGCATGG
TGTTGATCGTTTGTCAGGCCTTGGCAATACGCTTTTTCCAGTAGCGGATCTGGCGGCGCACTTCCTGCGGG
GCGGTGCCGCCGAAGGATTGGCGGCTTTTGACCGATTTTTCCACCGTCAGATAGCCGAAGACCTCTGCTGT
GATGGCGGGGTTGATCGACTGCAATTCCTCAAGCGAGAGCTTTGCCAGATCGACCTTGCGGCTTTCGGCAA
GCGCCACGGCGCGGCCCGTTACGTGATGGGCTTCGCGGAAGGGAAGGCCCAGTGTGCGCACCAGCCAGTCG
GCAAGGTCGGTCGCGGTGGAATAGCCGGAACCTGCGGCCTTCTTCATGGCTGCGACATTGACGGTGAGATC
GCGCACCATTCCAGCCATCGCGGCAATCGCCAGTTCCAGATTTTCGGCAGCGTCGAAGACCTGTTCCTTGT
CTTCCTGCATGTCCTTGGAATAGGCGAGCGGCAGGCCCTTCATGATGGTGAGAAGTGCGACCAGCGAACCA
TTGATGCGGCCCGTCTTGGCGCGTACCAGTTCTGCGGCGTCCGGGTTCTTCTTCTGCGGCATGATCGAGGA
GCCGGTGGAGAAGGCGTCGGAAAGGCGCACGAAATTGAACTGCGGCGTGGACCAGATGACGATCTCTTCGG
CAAGGCGCGAAAGATGCCCGGCGCAGATTGCGGCAAGCGACAGGAATTCCAGCGCATAATCGCGGTCGGAA
ACGCTGTCGAGCGAATTGCGGGTCGGCTCGCGGAAGCCCAGCGCCTTTGCCGTCATGTGACGGTCGATAGG
AAAGCCGGTGCCTGCAAGGGCGGCTGCACCCAAAGGCGATTCGTCCATGCGCTCGATGGCGTCGCGCACGC
GCGAAAGATCGCGGCCGAACATTTCCACATAGGCCATGCAGTGATGGCCGAAGGTGACGGGCTGCGCGGTC
TGGAGATGGGTGAAACCCGGCATGACTGTTGCCGCGTGTTCTTCCGCGCGCTCCAGAAACGCTTCGATCAG
GTTTTTGAGGGCGGCTGCGGTTTTTTCCAGTTCCTGCTTCACCCAGAGACGGAAATCGACGGCCACCTGGT
CATTGCGCGAACGGGCGGTGTGGAGGCGCCCGGCGGCAGGGCCGATCAATTCCGCCAGCCGCGCCTCGATG
TTCATGTGAATGTCTTCGAGCTTGCGCGAGAAGGTGAATTTGCCCTCTTCTATTTCCTTGCGGATGGTTTT
CAAGCCATTTTCAATTTGTTTGTGGTCTTCTGCCGCAATGATGCCCGTCTTTGCGAGCATGGCTGCATGGG
CGAGCGAGCCCTGAATATCCTGCGCATAGAGCTTGCGGTCGAAACCGAT
>BRA0077
ATGAAACGCTTCCGCATCGTTGCCCCCCTCGCACTCATGTCGCTTGCGCTGGCTGCTTGCGAAACAACAGG
CCCGGGCAGCGGCAATGCCCCGATCATAGCCCATACCCCTGCCGGCATAGAAGGAAGCTGGGTCGATCCGA
ACGGCATCGCTTCCTCATTCAATGGCGGCATCTTTGAAACCCGCACCACCGACACCAACGAAAAGCTGGCG
GAGGGCAACTATCTCTACCTGTCGCCGCAACTCGTTGAAATCAACATGCGGTCCATCGTGCGCGGCACCAC
CTCGAAGGTAAATTGCGCACTGGTTTCGCCGACGCAACTTAACTGCACCTCCTCGGCCGGTTCGCGCTTCT
CGCTCACCCGCCGCAACGCCGGC
>BRA0107
ATGTCGGCTCTACCTGATCATATTGAAGTAATTTCTAAAACATCTGATCTCAGGTTTGAAGACATCGATAT
CGTCGTTACACTTCCTACATTTCGGCGGCCCGATCATCTCATCCGTACCCTTGATATGCTGAAAGCGCAGA
AGACCGAAAAGCGCTTTGCGGTGATCGTGATCGAAAATGAGGCTGAAAAGAGGGAAGGGGCCGAGGTGGCC
GCTCCGCTGTTTGCGGATGGCACGTACCGGGGAATGCTGATTGTCGAGTCGCATCGCGGCAATTGCAATGC
CTATAATGCGGGCTGGCTTACGGCCaTGACCTGTTTTCCGAACTTCAAATATATCCTCGTCATCGATGATG
ATGAACTGGCCGATCCGATGTGGATCGAGAACATGGTTTCCGCGGCCGGGCGGTTCGATGCAAGCCTCGTC
GGAGGCCCGCAATATCCGGTCTTTGAAAAGCCGAATTCGCAACAATGGGCCAAACACCCGGTTTTTATGCC
GCCTTATACAAAGACAGGCGCTGTTCCGATCATCTATTCGTCGGGAAACCTGCTGATCGCGCGTCCTGTGC
TGGAGGCCATCGGCTATCCGTTCCTGGACCTGATGTTCAATTTTACCGGCGGCGGCGATTCCGATTTCGTG
GGCCGCTCCAAGGCGAAAGGTTTCCGGATTGCCTGGTGCGCGGAAGGTATCGTGCGTGAAACCATTCCGGC
GCGCCGTCTGGAAGGCGACTGGATCAGGGCGCGGGGCTTGCGCAACGGCGTTCTTTCCACTCTTACTGAAC
AGCGTCGGCGCAAGGATGAGCCTTGCGGGCAGATACGTGTGTTCCTGAAGAGCCTTGCGCTATTGGCTTAT
TCGCCGATCAAGGCCTTGCGGCGCGGTCTTGCGGCAGGCTTTGCGCCGGCTGGAATGTATTTTATCTATGT
TGGATTGGGACGGGTTTTGGCACATTTTGGATATCTGAATGAGCAATATCGTAACCCGGACAAGAAC
>BRA0135
ATGATCGCTGTCATTTTCGAAGCTTGGGTTCCTGGTGAAGCGCAAACTGAATATCTCGATCTGGCTGCGGA
GCTACGCCCGCTGCTGGCTGACCTCGATGGCTTCATCTCCATTGAGCGCTTCCAGAGCCTGACCACCCCCG
GCAAAGTTCTGTCGCTGTCCTTCTGGCGTGATGAAGCGGCTGTCGCAGCATGGCGCAACCTTCCTGAGCAT
CGCCGTGTGCAGGCAGCAGGCCGCGACCACGTCTTTGCCGATTACCGCCTGCGCATTGCAGAAGTCGCGCG
GGACTATGGAATGCGCGCGAGAGGCGAAGCTCCGACCGACAGCCGCAATGGCCATAAGGAGGACATCGATC
ATGTA
>BRA0158
ATGAACAAAGCGATCCTTGCAGTAGCAATGGTGCTCCTTCTGGCCGGTTGCGCCACCAAGCCCGAGGAAAT
TGGCCGCGCGCCCGACCTGTCGCCGGTCGCGGCCCATCTCGGCATGCAGAACAATCCGCAGTTCAACGGCT
ATCCGGCGCGGCCCGGCAAGGCGTCCTATTCGCTCTGGGATCAGCGTTCGACCAATTTCTTCAAGGACCCG
CGCGCGGCGACGCCGGGCGATGTGCTGACCGTCATCATTTCCATCAATGATCGCGCCAACCTCGATAACAA
GACGGACCGCGAGCGCGTGTCGAAGGGCATTTACGGCGGAGGCGGTTCATTCGCCACCAGCAGCATTACGG
GCGCGGCGGCTGGCGGTGATATGGATGCTTCGATCAACACTCATTCCGACAGCAAGTCGAAGGGCAAGGGC
ACGATTGAACGCAGCGAGGATATCCGCTTGCAGATTGCCGCCATCGTCACCGACACGCTGCCGAACGGCAA
CCTCATTATCCGTGGGTCACAGGAAGTGCGTGTGAACAATGAATTGCGCGTGCTGAACGTTGCCGGTGTCG
TGCGTCCGCGCGATATTTCCGGCAACAACACGATTTCCTACGACAAGATCGCCGAAGCGCGCATCTCCTAT
GGCGGGCGCGGGCGACTGAGCGAGATTCAGCAGCCGCCTTATGGCCAGCAGATTCTCGACCAGTTTTCTCC
TTTC
>BRA0203
TTGATGGAGAGTGGCATGTCCAAGCATAAAGGCCCAACCGGCGCGATGGTCGATCCTGAAAGCCTGCCTTA
CCGTCCCTGTGTCGGGCTGATGGTTCTCAACAAGGCGGGGCTGGTCTGGGCCGGGCGGCGCATCGTCATTC
CGGGCGATGAAATGGATGGCGCCACACAGCTCTGGCAGATGCCGCAGGGGGGCATCGATAAGGGCGAGGAC
CCGGCGCAAGCCGCGCTGCGCGAACTTTATGAAGAAACCGGCATGACGTCGGTCTCGCTTCTGGAGGAAGC
ATCGGACTGGATCAATTACGATCTGCCGCCGCATCTGGTGGGGCTGGCGCTCAAGGGAAAATATCGCGGCC
AGACGCAGAAATGGTTCGCCTATCGCTTCGAGGGCGATGAAAGTGAAATTGCGATCAATCCGCCACCCGGT
GGCCACACTGCTGAGTTCGATTGCTGGGAATGGAAGCCGATGGCTGACCTGCCGAACCTGATCGTGCCGTT
CAAGCGCAAGGTCTATGAACAGGTGGTTGCGACTTTCCGGCATCTGGCGGCC
>BRA0228
ATGGCATTTGTGCATTTTTCGAATCCCGGGAAGGCGCTGCACTATGCTTCCCAAACCTTTATCGGGTGTCT
CATATCCTGGTTCGCCCTGCGCGGCATAGGCATCGAGCACCCGATCTGGTCGATGATGACGGTTTTCATCG
TTTCCGATCCAGATATTCACACCACGCTGTCGCTTGCCCGCGTGAGGGCGATCAATACGGCAGTGGGCTGC
ACGATCGGCATGGTGATGATCTGGCTTGTCGGCTATTCGCCGGTCATCAGCCTTTTTGCCGCCGCGATAAC
GGTTGGCGTCGTCGTGATGATCGACAACTATCCGAGCAACTGGCGGCTGGCCCCGGCAACGGTAGTGATCA
TCATGGATGCGGGAAGTGCTGCCCAGACAAGCGATCAGGAATTATGGCTTGCCCTGTCACGGTTTCTTGAA
ATCACGCTTGGATGCGTGGTTGCGCTCTCGGTCGCGTGGATCTACAGCCGCACGATCATGACGATCAGAAA
GCGCCTGCGGCACGGCAAACCTGGGCAGACC
>BRA0236
ATGGCAAAAGCAGCGACCCCGAAAACTACCGCCGCGGCCGAAGCAAAGCCAGCGGCGAAGGCACCCGCCAA
AAAGGCGGCGCCCAAGACCACCGCAGCAGCCAAGCCCGCTGCTACGAAGTCTGGCGCCCCCAAGGCAGCAG
CAGCCGGTGCGATCGGCCACATTACGCAGGTCATCGGCGCTGTCGTCGACGTCAAGTTCCCGGAAGGCCAG
CTGCCGCTGATCCTGAACGCGCTTGAAGTGGACAATCAGGGCCATCGTCTGGTTCTCGAAGTTGCCCAGCA
CCTCGGCGAAGACACCGTGCGCACCATCGCCATGGACGCGACCGAAGGTCTCGTTCGCGGTCAGGAAGCAC
GCGACACTGGCGAACCGATCATGGTGCCGGTCGGCGTCGAAACGCTTGGCCGCATCATGAACGTCATCGGC
GAGCCGGTTGACGAAGCAGGCCCCATCAAGACCAAGGCAACCCGCGCCATCCACCAGAACGCGCCGGAATA
TATCGAACAGTCGACCGAAGCCGAAATTCTGGTCACGGGCATCAAGGTCGTCGACCTTCTGGCGCCTTACG
CCAAGGGCGGCAAGATCGGCCTCTTCGGCGGTGCAGGCGTCGGCAAGACCGTTCTCATCATGGAACTCATC
AACAACGTCGCCAAGGCGCACGGCGGTTATTCCGTGTTCGCAGGCGTCGGTGAGCGTACCCGTGAGGGCAA
CGACCTTTACCACGAAATGATCGAGTCGGGCGTGAACAAGCTCGGCGGCGGCGAAGGCTCCAAGGCAGCCC
TCGTTTACGGCCAGATGAACGAACCCCCGGGTGCCCGCGCCCGCGTTGCCCTTTCCGGTCTGACGGTTGCT
GAAAACTTCCGTGACCAGGGCCAGGACGTTCTGTTCTTCGTGGACAACATCTTCCGCTTCACGCAGGCAGG
TTCGGAAGTGTCGGCTCTTCTCGGCCGTATTCCTTCCGCTGTGGGTTATCAGCCGACGCTGGCAACCGACA
TGGGCGCCATGCAGGAACGCATCACCACGACGACCAAGGGTTCGATCACCTCGGTTCAGGCCATTTACGTG
CCTGCCGACGACTTGACCGACCCGGCACCGGCAACCTCGTTCGCCCACCTGGATGCAACGACCGTTCTGTC
GCGCTCGATCGCTGAAAAGGGTATCTACCCGGCTGTTGACCCGCTCGACTCCACGTCGCGTATGCTCGACC
CGAAGGTCGTCGGTGAAGAACACTATGCCGTCGCCCGTCAGGTTCAGTCGATCCTGCAACGTTACAAGGCC
CTTCAGGACATCATCGCGATCCTGGGCATGGACGAACTGTCTGAAGAAGACAAGCTCACCGTTGCCCGCGC
CCGCAAGATCGAACGCTTCCTGTCGCAGCCGTTCTTCGTTGCTGAAGTCTTCACCGGTTCGCCGGGCAAGC
TGGTCGATCTCGCCGACACCATCAAGGGCTTCAAGGGCCTTTGCGCTGGCGACTACGATCACCTTCCGGAA
GCTGCCTTCTACATGGTCGGCAGCATCGAGGAAGCACTCGAAAAGGCCAAGAAGCTGGCGGCTGAAGCTGC
C
>BRA0243
ATGGAATATTTCTTCCAGCAGGTGATCAACGGGCTTGCGCTCGGTTCGATCTATGGCCTGATCGCCATCGG
TTATACGATGGTCTATGGCATTATCGGCATGATCAACTTTGCTCATGGCGATATCTTCATGCTCGGCGCGT
TCATGGCGCTGATTGTTTTCCTCATTATCACGACCTTCACCGGAATGCTGCCGGTTGCACTGATGCTGCTT
TTGATGATGATCGTCGCCATGCTTCTGACGGGTCTGTGGAGCTGGGCGATCGAGCGCGTGGCCTATCGACC
GCTGCGTGGTTCGTTCCGCCTTGCGCCGCTGATCACGGCTATCGGCATGTCGATTGCCTTGTCGAACTTCG
TTCAGGTCACGCAGGGTCCGCGCAACAAGCCGGTTCCCCAGCTTCTGAATGGTTCCTACGAGCTGTTCGGC
AGCGGCGTCACGATCTCGCTCAAGCAGATCGTGGTGATCGTGGTTACGGCAATATTGCTCACGATCTTCTG
GTATGTCGTCAACCGCACCTCGCTGGGGCGCGCGCAAAGGGCTTGCGAGCAGGATCGCAAGATGGCGGCCC
TTCTTGGCATCAATGTCGACCGTGTCATCTCGCTCACCTTCGTGATGGGCGCCATGCTGGCGGCCGTAGCC
GGGACGCTTTACCTCTCCTTCTATGGCGTGATTTCGTTCGCTGACGGGTTCATCCCCGGTGTGAAGGCGTT
CACGGCGGCGGTTCTGGGCGGCATCGGCTCGCTTCCGGGGGCTGTTGTCGGCGGGTTGCTTATCGGTCTCA
TCGAAGCGTTGTGGTCGGCCTATTTCTCGATCGACTACAAGGACGTCGCCGCGTTCTCGATCCTGGCTATC
GTGCTGATCTTCATGCCGTCCGGCATTCTTGGCCGACCTGAAGTCGAAAAGGTG
>BRA0279
ATGAAACGCGCTCTGTTTCTTGCCCCCTTCTTCTTCGCCCTGCTTGCCGGTTGCTCGCCTGCGGAAAAGCA
GGAACCCGCCACACCTTTCGCACTGACGGAAAATGCCATGGGGCGCTATTGCGGGATGAACGTGCTGGAAC
ATCCAGGCCCCAAGGGGCAGATCATCCTGCGGCCGACCAACGAGCCGATCTGGTTCTCGTCTGCACGCGAC
ACGCTCGCCTTCACCATGCTGCCGGAAGAGCCGAAGAATATCGCCGCCATCTATGTGTCCGATATGGCAAA
GGCGCCCAACTGGGACGAGCCGGGTGCGAATAACTGGATCGATGCACACAAGGCTTTCTTTGTGATCGAAA
GCGGGGCCGTGGGCGGCATGGGCGCGAAAGAAGCCGTGCCGTTCTCTGACCGCGCCGCTGCTGAACGGTTT
ACAGCCGATAATGGCGGCCGGATCGTCAGCTTCGATGAGATGCCGAGGGATTACGTGCTGGGCGAGCAGAC
GGCCCAGCATGTGGAACCGGACGGCGAAAGCCATATGCAT
>BRA0322
ATGGCCCGCTCAGCCCTTCTCAACGTAATGGTACAGGCCGCCATGAAAGCTGGCCGCAGCCTGGCCCGCGA
TTTCGGCGAAGTCCAGAACCTCCAGGTTTCCCTCAAAGGCCCCGGCGATTATGTCAGCCAGGCAGACCGCC
GCGCAGAACAGATCGTCTATACGGAATTGAGCCGCGCCCGCCCGGATTTCGGCTTCCTGATGGAAGAATCC
GGCGAGGTCGAAGCCAAGGACGGACAGCATCGCTGGCTGGTCGATCCGCTCGACGGCACCACGAATTTCCT
GCACGGTATTCCGGTTTTTGCAGTCTCCATCGCGCTTGAGCGTCAGGGGCAGATCGTTGCAGGCGTCATCT
ACAATCCCGCCATGAACGAGCTTTACACCGCCGAACGCGGCGGCGGCGCTTTCCTCAACGATCGCCGTTTG
CGCGTCGCCTCGCGCAACAAGCTGGTCGATTCGGTCATCGGCACCGGCATTCCGCATCTGGGGCGTGGCCA
TCACGGCCATTATCTCGTCGAACTACGCAATGTCATGGCGGAAACCGCCGGTATTCGCCGCATGGGCGCGG
TTGCGCTCGACCTCGCCTATGTCGCAGGCGGCCGCCTCGACGGTTTCTGGGAAGAGGGTATGCACCCATGG
GATCTGGGCGCGGGCATCCTGATGGTGCGCGAGGCTGGCGGTTTCGTCTCCGATAAGAAGGGCGGGCAGGA
CATATTTTCCACCCAGTCCATCGTCGCAGGCAACGAAGCCATCCACGGCGCGCTCCTGAAGACACTCAAGA
AGCCGATC
>BRA0335
ATGAAAATCGGCATCATCAATACAAAGATCCGGACGGTCTTTTCGGCTTTCGCGTGTATGATTGCCGCAAG
CCTCGTCTGCACCATGCCGGCGCGCGCTGTCGTGGAAATCAACATCAACAAGGGTGTGATCGAGCCGCTGC
CGATCGCCATCACCGATTTTCTTTCGGCTGATCAGCTTGGATCGAATATCACGTCGGTCATTGCGGCCGAT
CTTGAGCGGTCCGGCCTCTTCGCCCCCATAGACAAGGGCGCTTTCATCGAAAAGATCTCCAATCCGGATGC
GGCGCCGCGTTTCGAGGACTGGAAGGTCATCAATGCGCAGGCGCTCGTTACGGGCCGCATCACCAAACAGC
CCGATGGCAGGCTCAAGGCTGAGTTCCATCTCTGGGATACGTTTGGCGGCCAGCAGATGATCGGCCAGCAA
TTTTTCACCACGCCGGATAACTGGCGTCGTGTTGCCCATATCATCGCGGACGCCATCTATGAGCGCCTGAC
CGGTGACAAAGGCTATTTCGATACCCGTGTCGTTTTCGTGGATGAATCAGGCCCGGCGCAGAAGCGTGTGA
AGCGCCTTGCCATCATGGACCAGGACGGCGCCAATGTGCGTTTTATTTCTGATGGCCGCGCCCTTTCGCTG
ACGCCGCGCTTCTCGCCGAACCGGCAGGAAGTGACCTATATGTCGTTTGAGGGCGGCTCGCCGAAGGTCTA
TCTGCTGCAACTTGAAACCGGCCAGCGCGAGCTTGTCGGCAACTTCCCCGGCATGACTATTGCGCCGCGTT
TTTCGCCCGACGGCCAGAAGGTCGTGATGAGCCTGCTGCAGGATGACGGCAGCGCCAATATCTATACGATG
GATCTGCGCAATCGCACGACCACGCGCCTGACCAGCAGCCAGGCGATCGATACGGGCGCTTCCTATTCGCC
GGATGGTTCGCAGATTGTCTTCACATCCGACCGTGGCGGGCGCCCGCAGCTCTATGTCATGGGTGCCGATG
GCTCCAATCCGCGCCGTATCTCGATGGGTGACGGCAGCTATTCCACGCCGGTCTGGTCCCCGCGCGGCGAT
CTCATCGCTTTCACCAAGCAATCGCAGGGCCAGTTCTCCATTGGTGTGATGAAGACCGATGGTTCGGGTGA
GCGTCTGCTGACATCGGGCTTTCACAATGAAGGCCCGACATGGGCCCCGAATGGCCGCGTGCTGATGTTTT
TCCGCAAGGCAGCCGGTGCAGGCGGCCCGAAACTCTTCACCATCGACCTGACCGGCCGTAACGAACGCCAG
ATCCAGACGCCGAATTTTGCTTCCGACCCGGCCTGGTCGCCGCTGCTCGAA
>BRA0350
ATGGCACCTCGCGTTCTCGTATCCGATAAGCTTTCGCCCACTGCCGTCCAGATTTTCAAGGATCGCGGCGT
GGACGTCGATTACCTGCCCGATCTCGGCAAGGACAAGGAAAAGCTTCTCGAAGTCATCGGCGAATATGACG
GTCTGGCGATTCGTTCGGCCACCAAGGTCACGGAAAAGCTGATCGCAGCCGCCAAGAAGCTGAAAGTCGTT
GGTCGCGCCGGTATCGGCGTCGACAATGTCGATATTCCGGCCGCTTCGCGCCGCGGTATCATCGTCATGAA
TACGCCGTTCGGCAATTCGATCACCACCGCCGAACACGCTATTGCGCTGATGTTCGCTGTCGCCCGCCAAT
TGCCGGAGGCCGACACCTCCACCCGCGCTGGCAAGTGGGAAAAGAACCGCTTCATGGGCGTGGAAATCACC
GGCAAGACGCTGGGTGTCGTTGGCTGCGGCAATATCGGCTCAATCGTCGCCACACGCGGCATAGGCCTGAA
AATGCATGTGGTGGCTTTCGATCCGTTCCTTTCGGATGCACGCGCGCAGGAACTGGGCGTTGAAAAGGTCG
AGCTGGATGAATTGCTGGCGCGCGCCGATTTCATCACGCTCCACACGCCGCTCACCGACAAGACCCGCAAT
ATTATCAATGCGCAAACGCTGGCGAAAATGAAACCGGGTGTGCGCATTGTCAATTGCGCGCGCGGTGGCCT
GATCGTGGAAAAGGATCTGATTGCGGCGCTGAAATCCGGCCATGTGGCGGGTGCTGGCATTGACGTCTATG
AAACCGAGCCTGCAACGGAAAACGAGCTTTTTTCTCTGCCCAATGTGGTCTGCACGCCGCATCTGGGGGCA
TCGACTTCGGAAGCACAGGAAAATGTCGCACTTCAGGTGGCCGAACAGATGTCGGACTATCTTGTGAAGGG
CGCCGTCTCCAACGCCATCAACATGCCCTCGATCACGGCGGAAGAAGCGCCGCGCCTGAAGCCGTTCGTGA
AGCTTGCCGATGTGCTGGGCGCTTTTGTCGGTCAGGTCACGGATGAGCCGATCCAGGAAGTGGAAGTGCTT
TTCGACGGTTCCACCGCCTCGATGAATACGCGTGCACTGCTCAGTGCTGCTCTGGCCGGTCTCATCCGTTC
GCAGGTTGCCGACGTCAACATGGTTTCCGCGCCAATCATGGTGAAGGAGCGCGGCATCATCGTCTCGGAAG
TCAAGCGCGACAAGTCCGGCATTTTTGACGGCTATATCAAGCTGACGGTGAAGACCACCGTGCGCGAACGC
TCCATTGCGGGCACCTGTTTCTCCGATGGCAAGCCGCGTTTTATCCAGATCAAGGGAATCAATCTCGACGC
CGAGGTCGGCCCGCATATGCTTTATGTCACCAACAAGGACGTTCCGGGCATGATCGGCCTTCTTGGCACGA
TATGCGGCAAACATGATGTAAATATTGCGAATTTCGCGCTCGGGCGCGACCATCCGGGTGGCGATGCAATC
GCAATGCTTTATGTGGATGAACAGATTCCACAGGCAGCCCTTGACGAACTGATGGCTCAGGAGGCAATCAG
GGCGGCGACACCGCTTGAGTTCAACGTCGGGGAAGTT
>BRA0384
ATGGTTTCTGTCCCGGCCTCGCGCCCTGCAAACCGCCTGATTGCGGGAGAAGAAAAGCGCTTGCAGCGGGC
TCTGGCCCGCACAGGCCTCTGGCTGCGCGATCATCAGCGCGCAATCCGCGCCATTCAGTGGGTGATTATCG
GGGCCTATGCGGGATTGCTGATTATCCCGCTCATGACACCGATGCCCGATCGCCTGTCCCATATCTGGTCC
AATGCGGTTCTGTTTGCCCAGTTCATGTTCTGGGGCATCTGGTGGCCTTTCGTACTGGTGTCCATGGTGCT
GATGGGCCGCACATGGTGCGGCGTATTCTGCCCGGAAGGTGCTCTGGCGGAAGCTGCCAGCCGCCACGGCT
ATGGTCGCGCCACCCCGCGCTGGATCAAATGGAAAGGCTGGCCCTTCGTCGCTTTTGCCTGCACCACGATC
TATGGGCAGATGGTGAGCGTCTACCAATATCCAAAGCCGGTCATCATCGTGCTTGGCGGCTCCACGCTTGC
TGCAATGGCTGTGGGCTTCCTTTATGGCCGTGAGAAGCGCGTCTGGTGCCGCTATCTCTGCCCGGTTAGTG
GTGTGTTTGCCGTGCTGGCCAAGCTTGCTCCCGTATATTACCGGGTGGATCGCGAGACGTGGAACGATTGG
GAGCGCAAACCGGGCACACCGATGCCGCATGTCAATTGCGCGCCGCTGGTGCCGATCAAGACAATGCGCGG
GGCTTCCGCCTGCCACATGTGCGGGCGATGCGCGGGTTTCAAGGATGCTATCCAGCTTTCGGGGCGCTCGC
CTTCTTATGAAGTCGTCGAGGTTGCAGGGGATGCCCCCAAACCATGGGAAAGCCTGCTTATCATTTTTGGC
ATGATGGGCATAGCCGCTGGCGCGTTCCAGTGGGCATCCAGCCCTATCTATGTCGATGTCAAGCAATGGCT
GGCCGAATGGCTGGTGGAGCACGGCGGCATCGCCATGATCCAGCCGCAGCTTCCCTGGTGGGTGCTGACCA
ATTATCCCGATCTCAACGACATGATGACACCGCTCGATGGTGGCTTGATGATCGCCTATATTCTCGCTGTC
GCTTGCATTACCAGCATTGCGGTGGGTGGGCTTGTGGCAATCGCCAATTGGGCGCTTGGCGGCTGGAAGGC
GGCGCGCTTCCACCATCTGGTGCAATGTCTTATCCCCATGGCGGGGTGCGGCGTGTTTCTCGGCCTGTCGG
CCACGACCGTGACACTTTTGAAAGCGGAAGATGTGCCTCTGGCTTTTGTGAGCGACCTGCGCATTCTTCTG
CTGTTGGGCGCCGTCGTCTGGTCGGCATGGCTCTTGTGGTCGGTCGCGCGCCGCTACACATCTTCGATGGC
TCGCAGGCTTGTTGCGACCGCCATGGGGGCGCTTGCGACGGGTGTAGCCGCATTCAATTTGGTATTGTTGT
TCTGGATCTGG
>BRA0416
TTGGTGAAGCGTATCAGGAACCGGAAACAGGCGCTGCTGATTGCGGCATTGCCGCTTCTTGTCTTTTGTGG
CCCGCCACAAGCCATGGCGCAGGATGTCAGTGCCGCGCGTCCGGCAGTGCTTCCCCTTCCCGGCGAAGCCG
AAGGAGCCAGTATCGGCTCCATTTCCGTTCGCCTGCGCCAATCCAGCGGCAATGCAGAACGCGACGCCGCC
GCGCAGGAAGCCGCACGCCGCATCACCAAATCCCTCGACGGATCGGAATTCAGCGCCATCGAAGCCGGCGA
AATTATTGCCGAACTCATTCGTCAGGAAGCAATCTCCGACGCCAGATTTTATCTGCTCCCATCTGCCTCGC
AAACCGGCGTCAAACTGGTTTTTGAAATCGACGCCACTTCAGGCACCAAGACGAAGCAAGAGCCAACCGGC
CTTTTTCTCGGCGACACATCCTCCTTTCCAGTCCTCTATGAAAGCAACCGCGCAGTACTGAGAACCATTAT
CGGCGCCGGATTGGGCGGCTATTCCGATGTGAATGCATGGTTCGGAAGGCCGGATCTGTTCAATGGCAGGA
ATCCGCTTGCTGGCAAATTGCCGGGCCGCTCAAACACATGGAACGAAGGCTATCTGGAGCTTGGCGTCGGC
GGCGCCGCGCAATTGGGCGAAAGCAACCTCTATGGGTTCGGCGCTGTCACCGGCATGTTTTCCTGGACCCT
CGGACAGGATATTTTTCGCGATGACGATCGCGGCTTCCTCGACTTCGAGAAAGCCTATGCCGGTGTGCTCT
ATGTATCGCCGAAGAACCGGAAAAACCATTTCAAATTCTCGGCCGGGCGGCAGGCTTATACACTCAATGAC
GGGTTTCTGGTGAATGCCGTTCGTGGCTCCACCAGCGCCGGCGAACGCGGCGGGCTTTATCTCGGCCCCCG
GCTCACCAACGACTTTTCGGTTCTGGCGCAAGGTGCTTATGATCGCTGGAACTATAGTGCCTTCTTCATCG
ATCCCAACGAAGTTGAATCGCTGGAATCCGACACCACTTTTCTCGGCGCAAATCTGCGTTACAACTTCACC
GACAAGTTTTCTGCCGACACTACATTAATAACGATCCCCAACTCGAAAACACAATTTTCCACCCCCTCTGG
CAGATTAATCCCGCGTGAAGGGCTGAACACGGTTTCAGGCCATTTGAAATGGACCAGTGCTTTCGAGATGC
CCGGCCTGTGGCTCGAAGGCGAACTGGCGCTCCAGTTTCACCCCGGTGAGGATGTGTCGGCCTGGGCCTAT
TACGGAACAGTCGGCTATCTCATGAAAGAGATTGGCTGGACGCCAAGTCTTTCCTATCGCTACGCCTATTT
TTCGGGCGACGATCCGAACTCATCGCGTTATGAGCGGTTTGATCCCCTGCTCTCGACCGGGCTTGGCATCT
GGTTGCAGGGTGTTTCGTTTGGAAAGATCACCACCAATTCCAATCTTGAGACGCACCGTGTCCAGTTCAAT
GTCGCACCCGACGAGCGGCTGAACCTGACATTCGACTACTATCTGCTGCGTACCCCCCAATTGAACAATCT
CGGTTCAAACCCGGCGCTTTCGACGCTATCGTCGCACGATCTTGGGCAGGAACTTTCACTTTCAGCACGCT
GGAGCGCGACCAAAAGCCTTTACCTGCAAGCACTTGTTTCACATGCCATTCCCGGCACGGCGCTACGCGAT
ATCGGTGCCGACCGGAACTGGACGACTTTTCAGGTCAGCCTCTACGGGGGCTTT
>BRA0421
ATGGATGGTTCCGTGGTCGTCAGCCAGTTGGGCGCGCGGATGCACTATGCGGTGCCTCGCATTTTTGCCAG
CCGCGAAAGGCTTGCCCATTTCTATACCGATATTTGCGCCTTGCAGGGTTGGCCGCGTTTTTTCAATGGCC
TCCCGCCAACCATGCTGCCCGCTTCGGTGCGCAGGCTTGTGGGGCGAATTCCGCAGGGTATTCCGCAAGAG
CGGATGACGACGTTTCCGGGTTTCGGCCTGCACTCTGCCGTGCGCAGGCTTTCCAACCAGACTGGGCCCAA
ATCGACTGCCAATGCAATCTGGGCGGGCAAGACCTTCGGCAAACTGGTGGCCGAAAGCGGCTTTCATGGTG
CAAGTGGCCTTTATGCTTTCAGCGGTGAGGCGCTGGAACTGCTGGCTACCGCAAAGCGCCAGGGATTGTGG
ACGGCTGTGGAGCAGATGATTGCGCCGCGCATGGTGGTCGATCAACTGGCTCTTGAGGAAGAAGCCCTTAA
TCCGGGCTGGCAGCATCCTGTTGCAAACGATGCACATGCCGATGTTTTTGCCGCACGCGAGCAGGCTGAGT
GGCGGATTGCCGATGTGGTGGTCTGCCCCTCGGCATTCGTGGCGCACCATTTGGCAGAAATGGGCTGTGCA
GCGGAAAAAATCGTCGTGGTGCCATATGGTGTCGATGATCGCTTTCTGCTCGCCCCCTGTCCTCGTCTGCC
GGGGCCATTGCGGGTGTTGACAGTGGGAGCCGTAGGCTTGCGCAAGGGCGCGCCTTATGTCGGGGCTGTGG
CCCGTATGCTTAAAGGTGAGGCGCAGTTCCGCATGGTCGGGCCAATTGAGATTCAAGCGGAAGCGCAGGTA
AAACTTGCTGCAAGCGTGGAACTGACCGGCGCTATTCCCCGCAGCGAAATGCGCGCGCAGTTTAAATGGGC
GGATGTGTTTTTGCTGCCTTCGCTTTGTGAAGGTTCCGCCACGGCGGTTTACGAAGCACTTGCCGCCGGCT
TGCCGGTCATCTGCACCCCGAATACGGGAAGCGTCGTACGCCACGGCATCGACGGTTATATCGTGCCGATC
CGCGATGTGCACGAAACGGCGCAAATCCTGCGGCAACTTGCCGATAACCCGGCACTGCTCGCGCGGATGAG
CGAAAGCGCGCGGGAGAGGGGTGCTGATTTCACCGTATCGCGCTATGGCGAACGGCTGGTCGCAGCGCTGG
CCCGTCACGACAGGAGGCAACGCGCA
>BRA0422
ATGACAGATTTTTACAGCCTCATCCCTTCGGCACCCAAGGGCCGCTTCGACGGCATTGAACGTGCGCATAC
GGCCGAAGACGTGAAAAGGCTGCGCGGTTCGGTGGAGATAAAGTATTCGCTGGCCGAAATGGGTGCAAACC
GCCTCTGGAAGCTGATCCACGAGGAAGATTTCGTCAATGCGCTGGGCGCGCTTTCCGGCAACCAGGCCATG
CAGATGGTTCGCGCCGGGTTGAAGGCGATCTACCTTTCCGGCTGGCAGGTTGCAGCAGACGCCAACACGGC
ATCAGCCATGTATCCCGACCAGTCGCTCTATCCGGCCAATGCCGGGCCAGAGCTTGCAAAGCGCATCAACC
GCACGCTGCAACGCGCCGACCAGATCGAAACCGCCGAAGGCAAGGGGCTTTCGGTTGACACATGGTTCGCG
CCCATCGTGGCGGATGCCGAGGCAGGCTTTGGCGGCCCGCTGAATGCCTTCGAGATCATGAAAGCCTATAT
CGAGGCCGGTGCTGCGGGCGTTCACTTTGAAGACCAGCTTGCTTCGGAAAAGAAGTGCGGCCATCTGGGCG
GCAAGGTTCTGATCCCGACCGCAGCGCACATCCGCAACCTCAACGCGGCACGCCTTGCAGCCGACGTCATG
GGCACGCCGACCCTCATCGTGGCGCGCACGGATGCGGAAGCAGCCAAGCTTCTGACGTCGGATATCGACGA
GCGCGACCAGCCTTTCGTCGATTACGAAGCAGGCCGCACAGCGGAAGGCTTCTATCAGGTGAAGAATGGCA
TCGAGCCATGCATCGCCCGTGCGATTGCCTATGCGCCTTATTGCGATCTGATCTGGATGGAAACGTCCAAG
CCTGATCTCGCACAGGCCCGTCGCTTCGCGGAAGCCGTGCACAAGGCGCATCCGGGCAAGCTGCTCGCCTA
TAACTGCTCGCCGTCGTTCAACTGGAAAAAGAACCTCGACGACGCGACGATTGCCAAGTTCCAGTGCGAGC
TGGGCGCGATGGGCTACAAGTTCCAGTTCATCACGCTGGCTGGCTTCCACCAGCTCAACTACGGCATGTTC
GAGCTGGCACGCGGCTACAAGGATCGTCAGATGGCGGCTTATTCCGAGCTGCAACAGGCGGAATTTGCAGC
CGAAGCCGATGGCTACACGGCAACCAAGCACCAGCGCGAAGTCGGCACCGGCTATTTCGATGCCATGTCGC
TCGCAATCACCGGCGGCCAGTCTTCGACCACCGCCATGAAGGAATCGACCGAAACCGCACAGTTCAAGCCG
GCTGCCGAG
>BRA0423
GTGGTCAATATCTATAACTGGTCGGATTATATCGACGATTCCATCCTCAAGGACTTCACCAAGGAGACCGG
GATCAAGGTCGTCTACGACGTCTATGACTCCAACGAAATTCTGGAAACCAAGCTTCTGGCGGGCGGCAGCG
GCTACGACCTCGTGGTGCCATCGGGCGAATTTCTTGGGCGCCAGATTCCCGCAGGCGTGTTCCTGAAACTC
GACAAGGACAAGCTGCCGAACCTCAAGAATATGTGGGATGAGATTTCGACCCGTGCGGCAACCTATGATCC
GGGCAACGAATATTCCGCCAATTACATGTGGGGCACGACCGGCATCGGCTACAATAAGGCCAAGATCAAGG
AAGCGCTCGGCACCGACACGATCGACTCCTGGGACGTGCTTTTCGATCCGGAAAAAACGGCAAAGCTGAAA
GATTGCGGCATTTACCTGCTTGATTCCGCCAGCGAAATGCTGCGTCCGGCGCTGAACTATCTGGGTCTCGA
CCCGAACTCTCCGTCGCCGGACGATTTGCAGAAGGCACAGGATCTCTATCTCAAGATTCGTCCGAATATCC
GCAAATTCCACTCGTCGGAATATATCAATGCGCTCGCCAATGGCGATATCTGCATGGCTGTCGGCTATTCC
GGCGATATTTTCCAGGCCCGCGACCGCGCCGAAAAGGCGAAGCAGGGGGTGGAGATCGGCTATTCGATCCC
GAAGGAAGGCGCGCTGATCTGGTTTGACCAGATGGCGATCCCGGCCGATGCCAAGCATGTGCCGGAGGCGT
TGGAATTCATGAATTATATGATGCGCCCGGAGGTGGCGGCAAAGGCGTCGAACTATGTGTTCTATGCCAAT
GGCAACAAGGCGTCGCAGAAATTCATCGACAAGGAAATCCTCGACGACCCGGAAATCTATCCGTCCGACGA
GGTGATGAAGAAGCTGTTCGTGCCGACGCCATATGACACGAAGACCCAGCGTGTGGTCACACGCGCCTGGA
CCAAGATCGTCACCGGCCAG
>BRA0427
ATGCAGAACAACTGGTCCCGCTTCAATATCGTATCGGTGGCGCTGGGTTTTGCCTTTCTCTACCTGCCCAT
CGTGCTTTTGGTGATTTATTCGTTCAATGAATCCCGTCTGGTCACGGTCTGGGCCGGGTTCTCGACCAAAT
GGTATGGCGAATTGTTCCGCAATCAGGCCCTGATGGATGCGGCCTGGGTGACGATCCGCGTCGGCCTTCTG
TCCGCGACGATCGCCACGGTTCTGGGCACACTCGCAGCCCTTGCGCTCACGCGTTATACCCGCTTTCGCGG
GCGCATCCTGTTTTCAGGCATGGTCTATGCGCCGCTGGTCATGCCCGATGTCATCACCGGCCTGTCGCTGC
TTCTGCTCTTCGTGGCGATGAATTTCGACCGCGGTTTCTGGACAGTGACACTTGCCCATATTACCTTTTCC
ATGTGTTTCGTGGCGGTCGTGGTGCAATCGCGCCTCGTCAGCTTTGACCGGTCACTGGAAGAAGCGGCGAT
GGACCTCGGCGCGCCGCCGGTCACGACCTTCATGAAGGTGACGCTGCCGGTGATCCTGCCTGCCGTGGTTT
CCGGCTGGATGCTTGCTTTCACCCTGTCGCTCGACGATCTGGTGATTGCGAGTTTCACCTCCGGTCCGGGG
GCAACCACGCTGCCGATGAAAATCTACAGCCAGGTCCGCCTTGGTGTGACGCCGGAAATCAACGCGGTCTG
CACCATTCTCATCGCGCTGGTGACAACGGGCGTCATTATCGCCTCCGTCATCAACAAGCGCCGTGAAGTGC
AGCGCCGCCGCGACGAACAGGCAGCATTCCGTATGGGA
>BRA0428
ATGACAAGTCTTTCCTGGATCACGCTTCTGGCTTTTGTCCTGTCGGTGGCCTTCTCCTTCTGCATGGCTTT
TCGTGGGCTTGCGCGCGAATGGTATCTGATCGACCTGCCCGATTACCGCAAAAGACATGAAGGTGCCGTCC
CGCTTTGCGGGGGTACCGCAATCTTCCTGTCCTTTTTCCTGACAGCTTCACTGGTAAAAGACGGTGCGGGA
CATGCAGATGCACTGGTGATGATGCCGGGGCTGACGATCATCCTCGGCACGGGGGTGCTGGATGATCGCTT
CAGGCTGCCGGTGACGCCGCGTTTTGCAATCCAGCTTTTTGCAGCGGTTCTCATCATCGCCATGGCGGGGG
TACAGCAGGTCTTTCTGGACCTCAACCCCTCCTTTATCGACGATAGCGGCAATATATTTGCCTTTGAACGG
CTGGGCGGCCCCTTTCTATTGCTTTTCGCCGTGGCCTTCATCGTCGGCCTTGTCAATGCGGTCAATATGAG
CGACGGCGTGGACGGGCTTGCAGGATCGGCAAGTGCAGCCTCCTTCTTCTGGCTCGCCGTTATCAGCTTCG
ACATCGGCGAACACCGGCTGGGCCTGCAATCGCTGGCGCTTGTCGCCGCCTGCCTTGGTTTCCTCGTTTTC
AACATGCGCCATAGGTGGCGGGCAAAGGCAAGCCTCTTTCTGGGTGACGGCGGCAGCACGCTTCTGGGGGC
CGCCCTTGCGGGAATGGTGTTGATCCTCTCCAGCGGCAGGCAAGCAATTGCTTTCCCTGTCTTGCTTTGGA
TCGTGATTGTGCCGGTCCTCGATACGCTTAGCCTCATCGTAAGAAGGCTCGCGGCGCGGCGCAGCCCTTTT
TCCGCCGACCGGCAGCATCTCCATCACCTGCTGATGGATGCCGGGCTGACCTGCGGGCAGACCGCAATCAT
CGTCATGGCGCTCAACCTGATTGCAGGCGCAACAGCCTATACGGCTATTCGCCTCGACCTGCCCGGCTGGC
TGATGGCGCTGGCGCTTCTCGCACCGGCCATGGCGCACCTAGCCTTCATCCTGCGTTGCACCAGAGGTCCG
CGCATCGCCGATCCCGCGCAGGCGGCAAAACCGAACATCACCTTTCCGGGGACCACGACA
>BRA0429
ATGAACAGGCACCGCAAAGGGTTCATCATCGCCACGCTTGAGCAATATGCCGGGCTGATGTCCAATTTCCT
GACCATGGTGGCTGTGTCGCGCCTTCTGGGCCCTGCCGAAACCGGCATAGGGGTGGTGGGCCTCGGCATTG
CGGCTATTTTCTTCTCCATGCGGGAATTTGCGAGCGCCGAATTTCTCATCAAGATCGATAAGGTCGAGGAT
AGGGATGTGCGGACATCGCTGACTTTGGTGATGCTGGCGACCCTGATCCTCGCTGCCGGTCTTTATCTGGC
GCGGGATTATCTGGCGACTTCCTATCGCGATCCCTCGCTCATGCTATTCCTGAATATCACCATTGTTGCGG
CGCTGGTGGAGAGCGCGTCCTTTCCGGTGGTGGCGCTGCTGCGGCGGGAAATGGCCTTCGGCGTTCTGGCG
TGGATCCGCACGGCGGGATCGACGTGCGGGGCGGCAACGACCATTTCCATGGCCTATCTCGGCTTTGGCCA
TATGAGCTTCGCCTTCGGGGCGCTGTCCGCCGCACTCATCACCACCGGCTTTGCCTTCAAGGCCTATCCGA
TGAAACATCTGCTGCGCCCATCTCTGGCGAGCATCGGCACCGCCTGGCAGTTCGGCCTTTATCTTGGCAGC
AGTGCGGGCCTGAACAAGATTTGCGAAACATTTCCGCAGCTTATCCTTGGAAAGTTCATGTCTGCGGCGTC
GGTCGGCATCTATAGCCGCGCTGGCGCTGTCAGCGGGGTTCCAGACCGCATATTCCTGTCGTCGATCTTTA
CGGTGGCTTTTCCCATGTTATCGGCACAGGTGCGCGAGGGGGCCGATATCCGTCGCTCCTACGTTCAGGCG
CTTTCCTATATTTCGGTGGTCTATTGGCCGGCACAGGCGGTGGTGGCGCTTCTGGCCTATCCGGCTGTGCA
TATCATTCTGGGTAAGGGCTGGATGGAAGCCGCGCCCATCGTGGCGCTCAGCAGCCTTGCAAGCCTGTTCT
GGTTCCCGGTCATTCCTCACCTATCCCTTGTTGATGGCACTGGGCGCAAATCG
>BRA0430
ATGAAGTGTTATCTTATTAATCTCGACAAGAGCCGGGATCGCCTCGAATTCATGGCGTCTCAGTTTGAGCG
CCTCGGCGCACAATTCAAGCGTGTGGAAGCCGTAAATGGACGAGCCATGTCGCCGCTTGAACTGGCTTCCT
TTACTCAAATAAGTAAAGAATGGCCCGCTCCTTTGTCGCCTGCCGAAATTGGCTGTTTTCTTTCTCATCGC
AAATGCCTTGAAAAGATTGCTGCCGGCGAAGATGCCTATGCGGCAGTCTTTGAAGATGACATTCGATTGAG
CCAGGGTTCCTCGCGGTTTTTGGCTTCAGATCACTGGATACCCAAGCAAGCGGATATCGTCAAAATCGACG
CTTACGGACATGAGGTATTGATTTCCAACCCTGTTAAAAATGAAG
GCCCGTATTCGATTTCCCGGCTGCGCTCGCGACACTTGCAGACGGGCGGATACGTTGTTTCGCGCGAAGCA
GCTCGCAAGCTCCTTCCATTGATGGAAAAAGCCTCAGCGCCGGTAGACCATTTCTTGTTCGATCCCAATGA
TGGGCCATTCAACGATTTTGAAATTTATCAGATTTCCCCTGCAATTTGCCGCCAGTCGGGAATGGAAAGCA
CAATCGGTCAGAATCGGCGCCCCAAACAGCGCCCCTCTCTTTTGGGCTTGGTCTGGCGTGAAGCTAAAAGA
TTGGTTATGCGAACCCGTCGCAATCTAAAAGGTTTCATAGCCAACGTCACCAAAACAGGCCGGTGGGGGCC
CATTCCATTTGATCGGGATATTGCG
>BRA0432
ATGCGGGGGCAGGCAATTGCGATAAACTGGCAGCGGTATATCTCATGCCCCCGATATACCGGAACCAGGCT
TCGAGGGAGCTTCTCCCCCGAAGTCCGGTTCCCGGAACCGTCACGGGGCAAGCCCGGTGTCCAGCTAAAGA
GCCTTGCCATGAAATGTGCCTACTTCGTGCGCCCCCATATTGGCGGAACCTATTCGGTTTTCACACGGTTG
CGTGCTTCGCTTGCCCGGTCTGGCATGGATCTGCGCTGGCTTGCAGCCGGTGCGCCGGGCGAAGCGGTGGC
CGTGCCGCGCGAGCAGGGGCTGGAAGATGCCATGCGCCGGGGCGACATCATAGATCGCATGGGCGTTCTGG
ATGAAGAAACCCGCGCCAAACGCTTTGTCGATTTTCTTCGCGAAAACAGGTTTGAAGCAGTTTTTGTGAAT
GTGCTTTCTCACCGTTTCGAGACCAATCTCGTGCGCTATCTTCCCGCGGATATTCTGCGTGTGATGATCGT
TCACAGCATCACGCCCGGCACCTATGCGGCGGCGCAGGCGATCCGCGATCATGTTCACGCGACGGTCGGTG
TTTCCAAACGGTGCCGCGACGATCTGGTTCGTGGCTATGGCTTCGACCCCAGCCGCACACTCGTCATTCCC
AATGGGCTGACGCCCGATCCGCATGTCCGGTGCGCAGCGCCGGAGATTGAAGGCGGACGGCTGCGACTGCT
CTATCTGGGGCGCATCGATGACAGTTCCAAGGGGGTCTTCTGGCTGCCCGCGATCCTGCGGCGGCTGCAAT
GCGATTATCATCTGACGGTGGCGGGCGACGGGCCGGACCTCGCAGCCTTGCGCATCAAGCTGGCTCCCTTT
GGCAACAGGATCAGTTTTACCGGCTGGGTCGCGCCATCCGATGTTTCATGGCTGGTGAGCCAGCATGACGT
GATGGTCATGCCATCGCGCTATGAAGGTTTCGGCTCGACGCTGATTGAGGCAATGAGCCAGGGATGCCCGG
TGGTGGCGTCGAGGATACCGGGCGTGACGGATACTATCGTCACCGATGGTGAAGACGGTTTTCTGTTTCCG
ATCGGCAATTGCAGGCAGGCGGCGCAGCAGATCGAGAGGCTTGCCGCCGATCCGCGCCTGCGCGTGGCGAT
GGGTGCGGCAGGCGTGCGCAAGGTGATCGCGGAATTCGACAGCGAAATCGTCGGGCGCGCCTATGCGGGAT
TGCTCCAGCATCTGCGTGAAAATCGCCCGCCCATTACGCCGCCTCTGGCTCTTTCGCAGTGGTCCATGCCC
AATGCCCTGCACAAAAACCTGCGCAGCCGCTTGCCGCAGCCGGTCAAGAACTGGCTGCGCCGCGTGCGGGA
GCGCGTGCACACGGTATGGTCGACGGCC
>BRA0463
ATGGATCACCACGCGGGCATTCCCGATCTGCGCCTTCACCGCATCCTCGATGCGGTCACAGATGACATGTG
CATCGCCGACGCTCATATCCGCCGCCACGACAAGGTGAAACTCGATGAAGGTCACGCGGCCCGCAATGCGT
GTCTTGAGATCGTGAACCTCGATAGCCCCACCCGCATTGGCCGAGATAACATTGCGGATACGCATTTCCTC
GGTCGGCTCGACCGCAATATCCATCAGCCCCTGGACCGAGGAATTGATGACGTGCCAGCCCTGCCAGAGAA
TATTGATGGCCACCAGAATGGCGAGCAATGGATCGAGCACCGACCAACCGGTTACCACCACGCCGACAAGG
CCGACCAGAACGCCCGCAGAGGTGAACACATCCGTCATGATGTGCTTGCCGTCGGCTTCCAGTGCTGGCGA
GCGGAATTTGCGTCCGGTGCGAATCAACAATGTCGCCCACAGGCCGTTTATGGCAGCAGCAGCCATGTTGA
TGCCGAGACCCAGCCAGGGTTCATCGAGCATTCGCGGTGTTTCCAGTGCAAACCAGGCCTCGCGGAAGATC
AAAAGTGCGGCGACCGTAATCAGAACGCCCTCCACCACAGCCGAAATATATTCCGCCTTGTGGTGCCCGAA
GGGATGATTGTCGTCGGCGGGCAGATAGCTGACCCGAATGGCCCACCACGCGCCGATTGCCGCAATCACAT
TGACAATCGATTCGAGTGCATCCGAATAAAGAGCCACGGAGCCTGTCAGCGCATAGGCCACATATTTCAGC
CCCATCACACCGAAAGCAACGGGAATAGACCAAGCGGCAAGCCGCCGAACCTTTGCACTCGCGTCCATAAT
GAACCTTTGATTATCTTGATCTTTCCCGAAACCGGGACGGGCCTACGCGTAAGCCCTTCATTCGTTCGCCA
GAGCGGTTCCTGTTTCAACGAAATCGCTGGAAACGCTCTAACTCTTTGTTTTGTCGCATTATCCGGCACAA
AACCGTTTCACACTTTTGCTGGAAATGCTCTATATCCCAGAAAAACAAAACTTCGTACCCTGTTTTTTGCG
TTCCAGCATTTAGAGCGCATCCCGAAAAGTGTGAAACGGTTTTCGGAAAAGATGCGCGTCAAAACAAAGGA
T
>BRA0472
ATGTCCGTATCCAAGAAATATATCCGCGGTGGCTTTGCAATCCTGATTGGCCTTTTGGGTGTTTTCATCAT
CTTGTGGGCTTGGCAGTTGCCGCCGTTCAAACACAGTGTCGAAACGACCGACAATGCCTATGTGCGCGGGC
AGGTGACGGTTATGAGCCCGCAGGTTTCAGGCTATATAACCAAAGTCAACGTGACCGATTACGAGCAGGTC
AAAAAAGGCGATCTGCTTTTTGAGATCGATAGCCGCAGCTATCAGCAAAAGCTGGATCAGGCGCTTGCCGC
GTTGGACAGCAAGAAGGCGGCACTTGCCAATTCGGAGCAGAGCCAGCGTTCCGCCGAGGCAACGATAAAGG
CGCGAGAAGCCCAGATATCGGGGGCAAAGGCGGCGCTTGAAGTGGCAAAAGCCAATACGGTTCGTGTCGAT
GCCCTGTTGCCGCGCGGTGTGACGACACAAAGCTCTGCCGATACGGCACATGGCAATATGCTTCAGGCGCA
GGCTGCGGTCGATCAGGCCGAAGCGGCACTGGCCGTTGCGAAGGAGGATTTGCGCACCATCATCGTCGCGC
GTGAAGGGCTCAATGCCGACCTGCACAATGCAGAGGCTGCGGTGGCGCTGGCCAGGATCGACCTCCAGAAC
ACGAAGATCCTTGCTCCAAGGGATGGCAAGCTTGGCGAAGTCGGTGTGCGGCTTGGCCAATATGTCACCGC
AGGCACACAGCTTGTTTCGCTCGTGCCGGCCGTCAAATGGATCATGGCCAATTTCAAGGAAACGCAACTTT
ATGGCATGAAGGTCGGCCAGCCGGTTACGCTGACGGTCGATGCGCTCCGCCATGCGGAACTGAAGGGGCAT
ATCGAGGCTTTTTCACCGGCAACCGGCTCTGAATTCAGCGTCATCAAATCCGACAATGCGACCGGCAATTT
CACCAAGATCACGCAGCGCTTGTCTGTCCGCATTTCCATCGACGAAGGGCAGCCGGATGCTGAATTGCTGG
CGCCCGGCATGTCGGTGGTGGTGCGTGTCGATACTGCCTCCACGCCGGATAAA
>BRA0473
ATGGTGACGGCCAGAATGCGCGATGCCGCCGTGCTTGTTCCGGTGGTGGACCGGGGGGCGGAGGCAACGCT
GCTGCTTACCCGGCGCACCGATACGCTGCGCAAACATTCCGGCCAGATCGCCTTTCCCGGAGGGGCCATCG
ACCCGGAAGACGGAACGGCGGAACGGGCGGCACTTCGCGAGGCCAATGAGGAAATCGGGCTTGCCGCAGAC
CGTGCGGAGATCATCGGCAACCTGCCGCGCTATCTCACCGGCAGCGGCTTTTCCATCACGCCTGTCCTGGC
GGTGGTGAAAACGCCGTTCGATGTGCATCCCAATCCGGACGAAGTCGCCGATATTTTTGAAGTGCCGCTTT
CCTTCCTGATGAATCCGGCCAATCATCGCCGCGAAAGCAGGCTGTTCAACGGCAAGGAACGCTTCTATTAC
GCCATGCCCTATCACGAGCGATTCATCTGGGGGGCAACCGCCGGCATCATTCGCGGACTTTATGAAAGGCT
CTACGTT
>BRA0497
ATGAACAATGCTCTCCTCGATTCCATCAAGAAGCGCCGTACGCAATATGCTCTGGGCAAGACCCTTCCCCA
TGCCCGGGAAGATGTTGCCGAACTGATCCGCGAAGCAATCAAGCACACGCCCTCTTCTTTCAATTCGCAAA
GTTCGCGCGCGGTGATCCTGTTTGGCCAGGAAAGCGACAAGCTTTGGAACATTGTGAAGGAAACGCTTCGC
CCGATCGTTCCAGCCGAAGCTTTCCCGCAGACCGAAGCCAAGATCGACGGCTTTGCTGCCGGTGCGGGCAC
GGTTTTGTTCTATGAAGACCAGAGCGTCGTGAAAGGCCTTCAGGAAAATTTCCCGCTTTATGCCGATAATT
TCCCGGTCTGGTCGGAACAGTCGGGCGGCATGGCGCAACATGCGGTCTGGGCCGCGCTTGCCAATGCGGGC
ATCGGCGCCAGCCTCCAGCATTATAATCCGCTGATCGACGCGGAAGTAGCCAAGACCTGGGATGTGCCGAG
CAACTGGAAACTCCGCGCCCAGATGCCGTTTGGCTCCAATGAGCAGCCTTTCCCGGAAAAGCAGTTCATGA
ACGACGCTGAACGCTTCAAGGTGTTCTTC
>BRA0529
ATGGCCGCGGCCATGCTTCTGCCCGCCGTAATCGATCTGCGTGACGGAAGCGACGACTGGTCGGTTTTCGT
GCGCTCTGCTGCCGCCACGGGTGCTCTTTCCGCGCTTATTTTTTTAGCGACACAGAACCAGACCATGCGTT
TCACGCCGCGTCTGGGCTTTCTTCTCACCGCCTGCCTCTGGCTGACGGCCGCGTTTCTGGGTTCCATACCG
CTTTACTTTTCGCATCTGCCGATCAGCTATGCGACCGCCTTCTTCGAGGCTATGTCGGGCGTAACCTCCAC
CGGAGCGACCGCGCTGACCGGCCTCGACAACATGCAGCGCGGCATTCTTCTGTGGCGCTCGCTCCTGTGCT
GGATCGGGGGCGTCGGCTTCATCGGCCTTGCGCTTTTGATGTTGCCCTCCTTGCGGGCAGGCGGCCTTGCG
CTTTTTCACATGGAAAATTCCGACAAATCGGAAAAAATCCTGCCGCGCATGAACCAGATCGCGCTCGGCAT
CATGGCCGCATATCTAAGCCTCACGGCTGCCTGCACGGTTGCCTATTTCGCTGTGGGCATGAGCGTTTTCG
ACGCCATCAACCACAGTCTCACCACCATTGCCACTGCGGGCTTTTCCACCCATGACAGTTCGATCGGCTTT
TATCAGGGCAACCGGATGCTGCTCGTCGTATCCACCATATTCATGGCGCTCTCAGCCCTCCCCTTCGTGCT
TTACATCAAGGCTTTCATGCCGCGGCGCATGGAATTACTCACCGACCCGCAGGTGAAACTCTTCTTCACCA
TCATCATCGCCTTCAGCTTCATCCTCGCCATCGTCCTGCGCCTCAGCACCAATATACCTTTCGGCGATGCC
CTGATCTCGGCCTCGTTCCATTTTGTCTCGGTGATAACGACGACCGGATATGCAACCGAGGATTATTCCCT
CTGGGGACCGCCAGCGATTGGAATTTTCTTTCTCGCCTCCTTCCTCGGCGGCTGTGCAGGCTCGACTTCCG
GCGGCATCAAAATGAACCGCCTCATCATCCTGTGGAGCCTGACGCAGGCCAATCTTGCGCGCCTCATCATG
CCGCATGCCGTCATCAAGATTCGCTACGGCTCATCGGAAGTTTCGGGCGATATCGCGCAGAATGTATTGCT
TTACCTGTTTCTTTACAGCGCCTCGCTCATTATCGGCGCGGTGCTGCTCGCATCCTTCGGCCTTGATTTCG
TATCCGCCTTCACGGGTTCGCTGACGGCATTATCGAATGTCGGTCCCGGCCTTGGCGACAAGATCGGCCCC
GCTGGCAATTTCTCCACCGTTCGCGATCAGGCGCTTTGGGTGCTCTCATTCCTTATGCTGGCAGGCCGACT
GGAACTCGTCACCATATTCATCCTCTTCACACGCTCATTCTGGGTGCGT
>BRA0544
ATGCTTCGCCAGGAGATTTCCCGGGCCCTCACAACCGCGCTGAAGGCGCAGGAGAAGCGCCGCATGTCAAC
GCTGCGCCTTGTCATGGCTGCCGTGAAGGACCGCGATATCGCCAACCGCACCGCTGGCAAGGACCCGGTCG
GGGACGAAGAATTGCTGGGCATTCTGGGCAAGATGGTAAAACAGCGCGAAGAATCCGCGCGCATCTATGAA
GAAGGCAGCCGTCTGGAACTGGCGGAAGCCGAGCGCGAGGAAATTGCCATTATTTCGGAATTCCTGCCGCA
GCAGATGACGGAAGACGAAACCAAGAAGGCCTGTGAGGATGTGATTACCGAAATCGGCGCGCAGGGCCTTC
GCGATATGGGCAAGTGCATGGCGCTTTTGAAGGAGCGCTATGCAGGCAAAATGGACTTTACCCGGGCAGGC
GCACTTGTAAAATCGCTGCTGCAA
>BRA0567
TTGCAGTCCGTCCTGCGGGAGGGAGGAACTCTTGCGGACGGTGGCCAGAGGGGAAACCGGGTGACGGAAAT
ATCCGCGCCCGGTTTCATAATTTCAAGCCCTGTTCTGCCTGTTGCGGATGGCCGCCCGGCCATTGGCGTTT
GCGCGAAGGCGTTTGCTTCTGCGCCGCATTTGGCC
>BRA0590
ATGCCGAAAACGACGGGGGGTGGCGTGGAGGCGCATGTGAAGGGAATGGGCTTCATGGTCCTTTCCGTGCT
CATGTTGCCACTGATGGATGCCATCGGCAAATGGCTTGCCATGATGGACGATTTGCCGCCTGCGACCGCCA
CTTTCATGCGGTTTTTCGTACAGTGCTGGCTGATGTTCTTCATCATCCTTGTTGTGGGTGGCTTGCGGGCG
CTGGCCACCTCGCATCTGATGGGCAATCTGCTGCGCGGCGCGTTGATGGGGTTCGGCGGCCTTTGCTTCTT
TACGGCGGTCAAATATATGCCGCTTGCCGACGCCATGGCGGTGTTTTTTGCCGAGCCTTTGATCCTGACGC
TGTTTTCAGCGCTTTTCCTGAAGGAGAAAGTCGGCTGGCGGCGCTTCAGCGCCGTTGGCATCGGCCTCATC
GGCACGATGATCGTCATTCAGCCCAGTTTCGAGATTTTCGGCATGGTTTCGCTTCTGCCGCTCGCAACCGC
TGTTACTTTTGCAATTTATCTGATCCTCAACCGCAAATATGGCGCGAAGGAAAGCCCGCTTACCATGCAGT
TGTATGCGGGGCTGGGCGGATGGCTGTTCGTGGGGGTGGCAATGCTGCTCGCTGCCGATAGGAGCTTTGAC
GATATGCATTTCGCCCTGCCGCACAGCGTACAGCCCTGGCTGCTCCTTTTGCTTTTGGGCACCATCGGTAC
GGTCAGTCACCTCATCGTGGTGCAGGCATTCAAGCTTGCGCCCGCATCCATGCTGGCACCGTTTCAATATC
TGGAAATTGTGAATGCCGTTCTGGTGGGACTGATTATCTTCGGCGATTTTCCAACGCCATCCAAATGGCTC
GGCATCGCCATCATCATTGGCTCCGGCTTTTACGTCTTCATGCGTGAAAGCAAGAAAAAAACAGGACGGAT
CTCA
>BRA0631
ATGACCGGCGCAGAAATGGTTATCCAGGCGCTGATCGATCAGGGTGTGGAAAGCATTTTCGGCTATCCCGG
CGGCGCGGTGCTTCCGATTTATGACGAGCTGTTTCAGCAGGATCAAATCCAGCACGTTCTGGTTCGCCATG
AGCAGGGTGCAGGCCATGCAGCGGAAGGCTATGCGCGCTCGACGGGTAAAGTCGGCGTCATGCTCGTCACT
TCGGGGCCGGGTGCAACCAATGCCGTGACGCCGTTGCAGGATGCCTTGATGGATTCCATCCCGCTGGTCTG
CATTTCCGGTCAGGTTCCCACGACGCTGATCGGTTCCGATGCGTTTCAGGAATGCGATACGATTGGCATCA
CGCGCCCCTGCACCAAGCACAACTGGCTGGTAAAGGACGTCAACGACCTGTCGCGCGTCCTGCATGAAGCT
TTCCATGTCGCATCCACCGGCCGTCCCGGCCCCGTCGTCGTCGATATTCCGAAGGATATTCAGTTCGCCAA
GGGCATCTATACGCCGCCGGAAACTGCACCGCGCACAAGTTACCGTCCAGTTCTCGACGGCAATGCCGAGG
CTATTTCCGAGGCTGTCCGTCTGCTCGCCAATGCCAGAAAGCCGGTCATTTATTCGGGCGGCGGCGTGATC
AATTCCGGCCCCGCGGCTTCCCGTCTGCTGCGTGAGCTGGTCGAGATCAGCAATTTCCCGATCACCTCCAC
GCTGATGGGGCTTGGCGCCTATCCCGCCTCCGGCAAGAACTGGCTCGGTATGCTGGGCATGCACGGCACCT
ATGAAGCCAACATGACCATGCATGATTGCGATGTCATGCTGTGCGTCGGTGCGCGTTTCGACGACCGCATC
ACCGGCCGCCTCAACGCTTTCTCGCCGAATTCCAAGAAAATTCATATCGATATCGATCCGTCGTCGATCAA
CAAGAACGTTCGGGTCGATGTGCCGATCATCGGCGATGTCGCGCATGTGCTGGAGGATATCGTCCGCCAGT
TTCGTGCTTTCGACCGCAAGCCAGACAGGCAGGCATTGACCGAATGGTGGGGGCAGATCGACCGTTGGCGC
GCGCGCAATTCGCTGGCCTATACGCCGAACAAAGACGTCATCATGCCGCAATATGCCATCGAGCGGCTTTA
TGAACTGACCAGGAACCGCAACACCTATATCACCACCGAGGTGGGCCAGCACCAGATGTGGGCGGCGCAAT
TCTTCGGCTTTGAACAGCCGAACCGTTGGCTGACTTCCGGCGGCCTTGGCACCATGGGCTATGGTCTGCCT
GCGGCGCTGGGCGTGCAGATTGCACATCCGGATGCGCTGGTTATCGATATTGCAGGCGATGCCTCGATCCA
GATGTGTATCCAGGAAATGTCGGCAGCAATCCAGCATAATGCGCCGATCAAGATTTTCATCCTGAACAACC
AGTATATGGGCATGGTGCGCCAGTGGCAGCAATTGCTGCATGGCAACCGCCTGTCGCATTCCTACACGGAA
GCGATGCCCGATTTCGTGAAACTTGCGGAAGCATACGGCGCGCACGGCATTCGCTGCGAGAAGCCGGGCGA
TCTCGATGCCGCCATTCAGGAGATGATCGATATCGACAAGCCGGTCATTTTCGATTGCCGCGTGGCAAACC
TTGCCAACTGCTTCCCGATGATCCCGTCGGGCAAGGCGCATAATGAAATGCTGTTGCCGGACGAAGCGACA
GATGAGGCCGTCGCCAATGCCATTGATGCCAAGGGCCGCGCGCTCGTC
>BRA0632
ATGAACGCACAAAACCTAGCTTCCGGATCGGCCTATTTCATTGCTGCCGAAACCCAGACGCCCGAAACCCA
CACGCTTGCAGTCCTCGTCGATAATGAGCCGGGCGTTCTTGCCCGCGTCATCGGCTTGTTTTCCGGCCGTG
GCTATAATATCGAGAGCCTGACGGTGTCGGAAACCGAACATGAGCGCCACCTTTCGCGCATCACCATCGTT
ACGCGCGGCACGCCGCATGTGCTGGACCAGATCCGCCATCAGCTTGCGCGCATCGTTCCGGTGCATCGCGT
GGTGGATTTGAGCCACCGCGCCGCCACGCTTGGCCATGAACGCCCGATCGAACGGGAGCTTGCATTGGTGA
AAGTGGCTGGAAAAGGCGAGGCGAGGGCGGAAACGTTGCGTCTGGCCGATGCCTTCAAGGCTAAGGTGGTG
GATGCCACGACGGACCATTTCATTTTGGAGATTACCGGAAAATCGGCCAAGATCGACCAGTTCATCTCGAT
TATGAAGCCGCTGGGTCTGGTGGAAATCTGCCGCACTGGCGTTGCCGCCATGAACCGTGGTGCGGAAGGCA
TG
>BRA0666
ATGCTTTACGGTATTTTACTTGCGTTTGCCTCCTATGCAGCATTCGCCGTCAGCGACGCCTGCGTCAAGTT
TCTGGACGGCACGCTTTCCCCCTATGAAATAGCCTTTTACGGCGCTGTACTGGGTTTACTTGCAATTCCCT
TCACCAAAAAAGCCGATGATGCATGGCTCGACATGTTCCGCACCGGCAATATCAAAATGTGGCTGCTGCGC
ACGGTCACGGCTGCAATGGGCGTCATCGGCAGCGTGGCAGCCTTCACCTATCTTTCCATGGCGGAAGCCTT
TGCGCTGATCTTCCTCCTGCCCGCCTATGTGACAATCCTGTCCGTGATCTTTTTGAAAGAACAGGTCGGCT
GGCGACGCTGGTCGGCGGTTGCTATCGGTTTTGCGGGCGTTCTGGTGGTGCTGCGCCCAGGCTTTCGTGAA
TTATCCATCGGCCATCTGGGTGCGCTGGCGGCAGGCATGGGCGGCGCGACGAGCATCATCATCTTCCGCGT
GATGGGAAAGAACGAAAAGCGCATTTCGCTCTATACGAGCGGGCTGGTCGGCCCCATTCTCATTTGCGGCG
CGCTGATGGCGCCCAGCTATCAAGCACCGGGCACGATGCAGTGGGTCTATCTTGCGGGCTACGGGCTTCTG
GCGGCTCTTGCCAATATATTGCTGATGCTGGCCGCGCAGCGCGCGCCCGCCAGTGCCATCGCCTCACCGCA
ATATAGCCAGATGATCTGGGCCATCCTGTTTGGATATTTCGTCTTCCACGACCATATCGACATGCCCATGC
TGATCGGCATCGTGCTGATTACGATTTCAGGTCTTTTCACGTTCATCCGCGAGCGCCAGCGCGGTGTGGAA
GGCCCGCCCGCCGTCGCGGCAGAGCCGACACCGGCTCTGGTCCAAGAAAAAGCGGGGTATTTC
>BRA0675
ATGAGCGAACCCAATTCCGAAACGAAGGCCGCAGCGGCGGCACCCGCCGATGAGCGGCTGAAATCGGCCAG
CACCTTGCAGCGCCTGTTGAGCAGGCCGGAACTGGGCGCGGTGTCCGGTCTGGTTCTCATCACGATCTTCT
TTGTTTCAACCGCCAATTCGGCCATGTTTACCGCAGCCGGGATCATGAATTTCATGGCCCCGGCCTCGCAG
CTCGGTATTCTTGCTGTCGCAGCGGCGCTGTTGATGATCGGCGGCGAGTTCGATCTTTCCATTGGGTCTAT
GGTGGCCTTTGCCGGCCTGATTTTTGCCGCAGCCCTTGTCGTCTGGCAGGTGCCGCTTGCCGTTGCCATTA
TCGTTACCTTGCTGTTTGCGGCGCTGGTGGGGGCGGTGAATGCGCAGATCGTCATCAGAACGGGTCTGCCG
TCCTTTATCGTCACGCTGGCCTTTCTCTTCATATTGCGCGGCCTTTCGCTTGTCGGGCTGAAGTGGGCAAC
CGGCGGTGCGACGCAGCTGCGCGGTGTGAAGGAAGCGGTCAGCGGCAGCTATCTGGCCGGCCTCTTTTCCG
GCGATGCATTTCCGGGGCTTTTCGCATGGCTGGCGGAAAAGGATTTGATTGCGAAATTTCCAAATGGCGAG
CCGGCAGTGAAAGGTGTGCTGGTCGAGGTGCTGTGGTTTGTCGTGATTGCCCTTGTCGCGACCTATATTCT
GGTGCGCACCCGCTTCGGCAACTGGATTTTTGCGGCGGGCGGCGATGCGCTTGCCGCGCGCAATTCCGGTG
TTCCGGTGCGCCGCGTCAAAACCACGCTCTTCATGTGCACGGCCATGGCGGCAGCACTTGTGGCTATCCTG
ACTGTTCTCGATGCCGGGTCCACCGATGCACGGCGCGGTTTCCAGAAAGAATTCGAGGCAATCATCGCGGC
CGTGATCGGCGGGTGCCTGCTGACCGGCGGTTATGGCTCGGCCATCGGCGCTTTCTTCGGCGCGATCATTT
TCGGCATGGTGCTGATCGGGCTGACCTATACGCAGATCGACCAGGACTGGTATCTTGTGTTTCTCGGGGGA
ATGCTTCTGATCGCTGTGCTCTTCAATAATTTCATCCGCAAGCGCGCCACGGGCGAGCGT
>BRA0737
ATGACGGGTTCCGGAAACCGCCTAACCAAACTGACGCTTGCACTTGTCCTCGGCCTTGTTGCCGTATTCAC
GGCTGTCGATTTCGCTGAGGCTCGCCGCGCGGGCGGTGGCGGTTTTGGTAGCCGTGGCATGCGCACCTATT
CTACGCCTGCGCCGACCCGTACCGCGCCCAACAATGCGGCTCCCATCGAACGCTCGATGACACCGAACACA
GGCGCGACGCAAACGACCCGTCCCGCTGCGGCTGGCGCACAGAATGCGGCACAGCGTTCCGGTGGCCTGTT
CGGCAATTTTGGCCGTTCCATGCTTGGCGGCCTGCTGGTCGGCGGCCTGATCGGCATGTTGCTTGGCAATG
GTCTCGGCGGTCTTGCCGGCATGTTCGGCCTGATCCTGCAAGTGGCCGTCATTGCCCTGATCGCCATGTTC
GTGATGCGCATGTTTGCCAATCGCCGCCAGACGGCAGCGGCTGGCGCGGGCCATGCCGGGGCGCAGCCTTT
CGGCGCGAGCCAGCCTTTCGGTTCGGCGAAAAGCGCATCCGGTCAGGGCAGCTTTAGCGGCCGCACACCAT
CCGTCAATCCGTTTGAGACCAAACAGGCTGCGGCTCCCGTCGTGGAGGATGAGCCGATGGATGTCGGACAG
GAAGATCTGGATCGTTTTGAGCAGATGCTTGTGGATATCCAGGGGGGCTATGGCCGTGAGGATTATGCGGC
GCTGCGCAAGCTCACCACGCCGGAAGCCATGTCTTATCTGGCGGAAGAACTTGGCGAAATCGCATCCGGCG
GCAAGCGCAATGAAGTGAAGGATGTGAAGCTTCTTCAGGGCGATGTGTCCGAAGCCTGGCGCGAAGGCAAT
GTGGAATATGCAACGGTGGCGATGCGCTATTCGGCCATCGACGTGATGGTGGATCGCACCAGCGGCGCGGT
TGTCGAAGGCAACCCGAATGAGCCGGTGGAAAGCGTCGAGCTATGGACCTTCACCCGCACAGATGGCGGCG
CGTGGCAACTCGCCGCTATTCAAGGCACGGAA
>BRA0758
ATGAAAAAGCTCCTTGCACATTCCGGCCGGGTTTTTCTCACCCTCGTCATGGTTGTTATGGCGGGGCTGCT
CGGCTGGCATCTCTGGGATTATTACATGAATGCGCCGTGGACCCGTGACGGCAAAATCCGTGCCGATGTGG
TGCGCATTGCGCCCGATGTCTCGGGCCTTGTGAGCGAGGTTCTGGTGCATGACAACCAGACGGTCCATAAA
GGCGATGTGATTTTCCGCATCGACCAGGCGCGCTACAAGCTTGCTTTGCAGACTGCTGAGGCCAAGGCTGA
CAGCAGCAAGGCAGCACTGGACATGGCCAATCGTGATCTTGTCCGCTATCGCCAGCTCAACGATACGACCG
TGACGCGCCAGAAACTGGAACAGGTAGAAACCGCAGACCAGCAGGCAGAGGCCGCCCACCGCCAGGCCGAA
CTCGACCGCGATCTGGCTGCGCTTAATATGGAGCGTTCGTCTATCAAGGCTCCGGTCAATGGCGTTATCAC
CAATTTCTCGCTTCAGCCGGGCGATTATGTTTCGGCGGGTTCGGCCGTTACGGCGCTGGTCGATACGGATT
CCTATTATGTCTCCGGTTATTTTGAGGAAAACAAGCTGGAGCGTATCCATATCGGTGATCCGGTGGTGGTC
GATGTGATGGGCAGCAAGGTGCAGCTCAAGGGTAAGGTGGAAGGTATTGCGGGCGGCATCGAGGATCGCGA
GCGCAGCGATTCCCAAAGCCTGCTCGCCAATGTCTCGCCGACGTTCAACTGGGTGCGCCTTGCGCAGCGCG
TGCCGGTGCGTGTGAAACTGGAAGATGTGCCGCAAGGCATCCATCTGGTGGCAGGCCGCACGGTGAGCGTT
TCGGTGGTGGAT
>BRA0767
GTGATCGGATTAAAATGCTTGAGTGCCATTGTCTCTTACTCCATGGCCTCTCAGAGACCGGTCGAAACGTC
GATGCTCTGGCCTTCGGCCAAAGTAACGATCGCTTTCTTGACGTCGCTCTGGCGCCCGACAAGGCCGCGGA
ACCGCTTAACCTTGCCTTTGCGCACTGCAGTGTTGACTGCCGTAACCTTCACGCCGAAAAGCGCTTCGACT
GCAGCCTTGATTTCCGGCTTCGTCGCCTTGCGGGCCACGTTGAAGACGACCTGGTTGTGTTCGGAAACCAT
GGTGGACTTCTCGGTGATGACCGGGCTGACGATCACGTCG
>BRA0791
ATGCTCTATTGCGAAACATCCAATGCCAATATGGCAGGCGCAGCCCTCTTATATCGGGGCTGCGCCTTTTT
TCTTGCCGAATCGATAATGCCGGATTTCGTGCAGATTGCAGAAGTTCGTGGA
>BRA0816
GTGCGCGAATATTATACGATAACGGAATTGACGAGGGAGTTCGGAATTTCCACCCGGACGCTTCGTTTCTA
TGAGGATGAAGGGTTGATCGCTCCGGTGCGGCGCGGTCGTACACGTCTATTCCGACCGTCCGACCGACATC
TTCTCAAGCAGATCATGCGTGGCAAGCGATTGGGCTTTTCTATCGCGGAAATCCACGAAATCATCCAGATG
TATCGTGAGCCACCGGGGGAGACCGGGCAGCTCAAGCTGCTGATGAAGCGCGTGGAAGAAAAGCGCGCCGA
TTTGCGCCAGAAGCGCCGGGATATCGATGAAACGCTTGGGGAACTCGATCAAGTCGAGGAAGCCTGCATAG
AGCGTCTGGCGGAGCTTGGCGTATCGACC
>BRA0853
ATGTCGAAACCGGTTATTACCCGTTTTGCCCCCTCGCCGACGGGTTATCTTCACATTGGCGGAGCCCGCAC
CGCTCTGTTCAACTGGCTTTACGCCAAGCATTGCGGCGGCAAGATGCTGCTGCGCATCGAAGATACCGACC
GCGAACGTTCCACGGAGGCTGCAACGGCAGCCATTCTGGACGGCCTGACCTGGCTTGGCCTGGATTGGGAT
GGCGAGGCAATCTCGCAGTTCGAGCGCGCGCCGCGCCACCGCGAAGTGGCCGAAGAGCTGGTTGCAAACGG
CAAGGCCTATTATTGCTATGCAAGCCCGGAAGAGCTGGAAGAAATGCGCGAGAAGGCACGCGCCGAAGGCC
GCCCGCCGCGTTATGACGGGCGCTGGCGCGATCGCGATCCCTCCGAGGCACCGGCAGGCGTAAAGCCGGTC
ATCCGAATCAAGGCGCCTCGGGATGGCGAAACCGTCGTGCACGATGCCGTGCAGGGCGATGTTCGCTTCCC
GAACAAGGATCTGGACGATTTCATCATTCTCCGTTCGGATGGCACACCGACCTATATGCACGCTGTCGTGG
TCGATGACCACGACATGGGCGTCACCCATATCATCCGCGGCGGCGACCATCTCACCAACGCCGCGCGCCAG
ACGATCATCTATAATGCCATGGGCTGGGACGTACCGCAGATGTCGCATATCTCCCTCATCCATGGCGCCGA
TGGCGCAAAGCTTTCCAAGCGTCACGGCGCGCTTGGCGTGGATGCCTATCGGGCCATGGGCTATCTGCCGG
CCGCCCTGCGCAACTATCTCGTCCGCCTCGGCTGGAGCCATGGCGACGACGAAATCATGTCAACCGAACAG
ATGATCGAATGGTTCGATGTGAAGGACATCAACAAGGGCGCTGCCCGCTTTGATTTCCAGAAGCTGGAGGC
AATCAACGGTCTTTATATGCGTTCAAGCGACGATCAGGCGCTGTTTGATGCGCTCGTTGCCGTTTTGCCGG
AAATTGAAGGCGGCAAGGAGCTGGCCGAAGCCCTGGACGACAAAGGCCGCGCACAACTTCTGCTCGCTATG
CCGGGGCTGAAGGAGCGCGCAAAGACGCTTGTTGAGCTTGCCGATGGTGCAAAGTTCATTTTTGCCAGCCG
CCCGCTCGCGCTGGACGAAAAAGCCGCATCCTTGCTCAACGATGAAGGCCGCGCTGTACTGAAGCCGGTCT
ATCCTGTTCTGGAAGCAGTGGGCGAATGGACGGCTGAATCGCTTGATGCGGCAATCCGCGCCCATGCGGAA
GCCGAAGGGCTGAAACTTGGCAAGATTGCGCAACCCTTACGCGCCGCCCTGACTGGGCGCGCCACTTCTCC
GGGTGTATTCGACGTATTGGTGGTGCTTGGAAGAGAGGAATCCCTCGCCCGCATCGGCGATCAAATCGGT
>BRA0915
ATGATCGACGATATCTACAATAAACGCATCCTGGAGTTTGCGGGCAATATGGAACGCATCGGGCAGCTTGC
CGAGCCGGATGCCGTTGCCACGGTGCATTCAAAACTATGCGGCTCCACGGTTACCGTTTATCTGAAGATGA
GGGATGGTGTGGTGACAGATTTCGCCCACGAGGTGAAGGCATGTGCGCTGGGGCAGGCGTCTTCCTCCGTC
ATGGCGCGCAATGTGATTGGCGCCACCGCCGACGAATTGAGGGCGGCCCGCGATGCCATGTACAGGATGCT
GAAGGAAAACGGCCCGGCACCGGAAGGGCGCTTTGCGGATATGAAATATTTTGAGCCGGTGCGCGACTACA
AGGCCCGCCACGCTTCCACGCTTCTTACCTTCGATGCTGTCGCTGATTGCATTCGCCAGATCGAGGAAAAG
GCGAAAGCCGCC
>BRA0916
ATGGGGTCTTGCGGCGGGAAACATACGGGCAAGGGCGCGCCTAAACCTTACAGCCGCAACTTCACCGATCC
ATGGCGCAAGACGCCGGGACGCCTGTTCGGCACGGCACTGATACGCTTTTACCAGATTACGCTCTCATCGC
TTATCGGAAACTCCTGCCGGCATCTGCCGACCTGTTCCGAATATGCCTATGAAGCGATTGCGCGCCATGGT
CTTTGGCGCGGGGGCTGGATGGGGTTCTTTCGCGTGGTGCGCTGCGGACCGTTCGGCACGCATGGTTTCGA
CCCCGTGCCGCGGGAACTTTCTCCCGATCTCAAATGGTATATGCCCTGGCGCTATTGGCGCTGCTCGGCTT
CGCGCACCGGAAAA
>BRA0921
ATGAAATCATCCCGCAATATTGATCGTTTGTTGGAAATCATGGCCGCCCTTCGCAATCCCGAAACGGGTTG
CCCGTGGGATATCGAGCAGAATTTCCGCACCATCGCGCCCTATACGCTCGAAGAAACCTATGAGGTTCTCG
ACGCCATTGAACGCGACGATATGGATGACCTGCGCGAGGAACTTGGCGATCTTTTGCTACAGGTCGTCTTC
CATGCCCGAATGGCGGAAGAACAGGGCTATTTTGCCTTTGGTGACGTCGTGGAAGCCATCACGCATAAGAT
GATCCGCCGCCACCCGCATGTTTTCGGCGATGCGCAGGCACGAAGCGCCGGAATGGCCAAGGGTTCATGGA
ACCGCATCAAGGCGGAGGAAAAGGCAGAGCGCGCGGAAAGGCGTGCAAAACTGGGACTGGAAAGCATCGGG
AAAACCGGCTTTCTGGATGACATTCCCAACGCCTTCCCTGCCCTTCTGCGCGCGCTGAAATTACAGCAGAA
AGCGGCAAAGGTCGGCTTTGACTGGTCTGAGGCAGCCCCCATCCTCGACAAGATTGCCGAGGAAACAGCGG
AATTGAAAGAAGCCATGGCCGCAAGCGACAAGGCCGAAATCGAGGAAGAATATGGCGACCTTCTCTTCGCC
ATGGTCAATCTTGGCCGTCATCTTGAAATCGATGCCGAAACGGCGCTGATTTCCGCAAACGAGAAATTCAA
ACGCCGTTTTCATTTCATCGAGGAGGCTTTGAAAACGTCAGGCCGCGATCTCGATGCAGCCAGCCTTGATG
AAATGGAAGAAATCTGGAGCGAAGCGAAGAAAAAAGGCCTG
>BRA0948
GTGAGCACTCCGCCGAAACCATTGCGCGCCTGGCTTGGCCTCGGCGGCAATATCGATGATCCGATCATCTC
CATGGCGAGCGCGCTTCGTATGCTGGATGGACGGaGCGACACGAGGGTTGTTTCCGTATCCCCGGTCTACC
GGACCCCGCCATGGGGCAAGACGGATCAGGCCTGGTTCCACAATGCCTGCGCGCAAATCGAAACGGCCTTG
CAGCCACTGGAACTGATTGCGGCCTGTCTGGACGTGGAGCGATCCTTGAAACGTGTGCGGCTGGAACGGTG
GGGACCACGCATCATCGATATCGATATTCTTGTTATGCAGGATGAGACGGGCAGGGCAATCACGATGCAGG
AACCGGCATTGGAGCTTCCGCATCCACGTATGCACGAGCGAGCcTTCGTtCTGGTGCCGCTCAATGATATC
GCACCTTCGCTGCCTGTCGGTGCGAGAACTGTTGCTGAATGGGCTGCCGATTGCGACCGTGCGGAAATGGA
AAAAGCCCGCACGGATGCGGGCTGGTGGTTGGAA
>BRA0960
ATGTCGTCAGTCCTTTCCCGCTATGCGCTTACCCGTCGCGCGGGCCTGAAAGCCCTTCTCTTCACCGCTGC
GGCCCTCACCGTCGGCTTTGCATCCGCTCCCAGCCATGCAGAAGACAAGACCATCAAGGTCGGCATCATGG
GCGGCGAGGACGAAGATGTCTGGAAAGTCGTTGCTGAAGAAGGCAAGAAGCATGGCCTCAATATCGAGCGC
ATCACCTTCAACGATTATAACCAGCCGAACGAAGCCCTGGAGCGCAAGGAAATCGACGCCAACGCATTCCA
GCACAAGCCCTATCTGGACGAGCAGATCAAGCAGCACGGCTACAAGATCAGCGTTGCAGGCTATACCGCAG
TCTGGCCGATCGGCATTTATTCCCGCAAGGTGAAGAAGCTTGAGGAACTGAAGGACGGCGCCACCGTTGGC
GTGCCAAACGACCCGACCAATGAAGGCCGTGCGCTGCGCGTGCTGGAAGAACAGGGCCTCATCAAGCTGAA
GCCGGATGCGGGCATTCTGGCGACGCCAATCGACATCGTTGAAAACCCGAAGAAGCTTGAAATCAAGGAAC
TGGACGCCGGCGTCGTCGGCCGCTCCATCGATGATCTTGACGCGGCAATCGTCAACAATGACTGGGCTGCG
AAAGCCGGGCTGAAAAAAGAGGATGCCATTGGCTGGGAATCGAAGGAAAACAATCCCTACAACAATTTCAT
CGCCGTTCGCACCGATGATCTCGACCAGCCTTGGGTGAAGGCACTGGTTGCCTCATTCCAGAACGACGCCG
TGAAGGCCGAACTTGAGCGCGCCTACAAGGGCACCGGCATCCCGGCCTGGAAT
>BRA0999
ATGACGCCGGAGACTGCATTGATCAACGAGTACCTCGCCAAGCATGGTGCACGCCGGTTCGAGCAGGGCGC
CACCAGTGGCATTCACGGGATAGCGTCATTCATGGCTGAATATGGCTACGAGGTAGCCGGAGCACCGAAAG
GCGGCGTCAAGGTTCGTCGTGGCAAAGGCCAGTGGAAACGTATGTCCATGCCAGGGCTAATCGCAATGGCA
GATGAAATTCGCCTTGCGCAGGGGCTTGAGCCTTTCAGCGCAGCGCACAAACAGGCAGCA
>BRA1047
ATGAAAAATGCGGCTTCCATTACGATAGCGAGCGGTATTATCGGCTTGGTTGCGCTTGGTATCGGATATTG
TGCCGGTACGGCGCGACAGGCCTCCAAGCCTGTACAGGCTGCAACCTCCACCGTTGCCTTTAACCCGAAGG
CCGTCGAAAACATCGTGCGCAATTATCTGCTTCAGAACCCGGAACTGATGCTGGAAGTGCAGACCGCGCTT
GAAACCAAACAGGCTCATGCGGCACAGGAACAGGTGAAGCAGGTACTGGCCGCCAATCAGAGCGTTCTTTT
CGACCCCAAGCATGATGCTGTCTTTGGCAACCCGAATGGCGACGTGACGGTCTATGAATTCTTCGATTACA
ATTGCGGCTATTGCAAGCGCGCGCTTCCCGATATGGAAGCGATCCTGAAAAAAGATCCGAATGTTCGTTAT
GTCCTCAAGGAGTTCCCGATCCTGGGGCCGGATTCCATGCGTGCGCATGTGGTCTCCCAGGCTTTCAAGGC
GCTGATGCCGGAGAAGTACCCGGAATTTCATGAAATGCTGCTTGGCGGGCATGGGCGTGCAACGGAGGAAT
CCGCGATTGCCGACGCCGTAAAGCTCGGCGCCGATGAAGCTAAGCTTCGTGAAAAAATGAAGGACCCGGCC
ATCACCGGCGCTTTCCAGCGGACCTACCAGCTTGCGCAACAGCTCAACATCACCGGCACTCCGTCCTATGT
CATCGGCGACGAACTGGTGCCCGGCGCTATTGGTATCGATGGATTGCGGCAGAGAATCGCGGCCGCCCGGG
ACGCTGCAAAGAAG
>BRA1050
ATGTTTCAAAAGATACTCATAGCCAATCGTGGCGAAATCGCTCTTCGCGTGCTCAGGGCTTGCAAGGAGCT
GGGCATAAAGACGGTCGCCGTTCATTCCACGGCAGATGCCGACGCAATGCATGTGCGCCTGGCAGATGAGA
GCGTATGCATCGGACCGCCGCCCTCACGCGACAGCTATCTGAACATCCATCAGATCGTGGCCGCCTGCGAA
ATCACCGGCGCGGATGCAATTCACCCCGGCTACGGCTTCCTTTCGGAAAATGCAAAATTCGCCGAAATCCT
TGAAGCCCATGACATTACCTTCATCGGCCCCACTGCTTCGCATATCCGCATCATGGGCGACAAGATCGAAG
CCAAGCGCACCGCAAAGCGCCTCGGCATCCCGGTCGTTCCGGGTTCGGACGGCGGCGTGACGGATGAAGTC
GAAGCAGCCCGTATCGCCAAGGAAATCGGCTATCCGGTCATCATCAAGGCTTCGGCCGGCGGCGGTGGTCG
TGGCATGAAGGTCGCCCGCAGCGCGGAAGAGCTTCCGATCGCACTTGCGACGGCACGTTCTGAAGCTGGCG
CGGCCTTTGGCGACGATGCGGTCTACATCGAAAAATATCTCGAAAAACCGCGCCATATCGAAGTTCAGGTC
ATGGGGGACGGTGCGGGCAATGCCGTCCATCTGGGCGAACGCGACTGCTCGCTGCAACGCCGTCACCAGAA
GGTCTGGGAAGAGGGCAACTCCCCCGCCCTCAATGCGGAAGCGCGCGAAAAGATCGGCATGATCTGCGCCA
ATGCCTGCGCAGAGCTTGGCTATCGCGGCGCAGGCACGATCGAATTTCTCTATGAAGATGGAGAATTCTAT
TTCATCGAAATGAATACGCGCCTTCAGGTGGAACATCCGGTTACGGAAGCGATCACCGGCATCGATCTTGT
GCATGAGCAGATTCGCGTTGCCGCAGGCCTTGGTCTTTCGGTGAAGCAGAAGGATATCCGCTTTTCCGGCC
ATGCCATCGAGTGCCGTATCAATGCGGAAGATCCGCTGACCTTCGCACCGTCACCGGGCCTCATCACGCAT
TATCATACGCCGGGCGGGCTTGGCGTACGCGTCGATTCCGGCGTTTATTCCGGCTATCGCATTCCACCCTA
CTATGACAGCCTGATCGGCAAGCTCATCGTGCATGGACGTAACCGCGTCGAATGCATGATGCGTCTGCGCC
GCGCGCTGGATGAGTTCGTGGTGGATGGGGTAAAGACCACCCTGCCCCTGTTTCAGGATCTGATTTCCAAT
CAGGATATCGCCAATGGCGATTATGACATCCACTGGTTGGAAAAGTACCTGGCCAACAAGAGCAAGAACGA
AAGCGCC
>BRA1053
TTGGGTTTTTTCCTGAAAAGCCTTGAGGATAGAGCGGATGCTTTGGGCCTGCTGCCGCTTTGTGGCTTGCA
GCAGGGCAACGAATCGTCACATTTCATGCTTAGGAATTATTCCGGCTTTATT
>BRA0063
TTGTGTAACGTCCTGTCACGTAAGGATATTATCTGTTCAAACGCTAACCAGGCAGGTTCGGTCATGGGAGG
TAAAACGGTCACGCGGGCTGATCTGGCAGAGGCTGTTTACCGTAAGGTAGGCCTTTCCAGAACGGAATCGG
CGGCTTTGGTCGAGATGATACTCGATGAAGTCTGTGACGCTATCGTCAATGGCGAAACGGTGAAATTGTCG
TCTTTTGCGACATTTCAGGTTCGCGACAAGAACGAGCGTATCGGTCGTAACCCCAAGACGGGTGAGGAAGT
GCCGATCCTCCCGCGGCGTGTGATGACGTTCAAGGCGTCTAATGTTCTCAAGCAGCGCATTTTGCAGGAAC
ATCAGAAGCGTCAGGGTAAAACCTCGAAG
>BRA0058
ATGATAAGATCTGTCGTACGGGGTATCGGTTCGGCATTGCCTAAACGGGTCATGAAGAATACCGACTTTGA
AGGCATTGTCGAAACCTCGGACGAGTGGATCGTGCAGCGCACGGGTATCCGTGAACGCCACATTGCGGGCG
AGGGCGAAACGACGGTTTCGCTGGGAGCGGCTGCTGCGCGCGCGGCAATCGAGAATGCCGGGCTTCAGCCG
TCCGATATCGATCTGGTCCTTCTGGCAACCTCCACGCCCAACAATACCTTTCCGGCCAGTGCAGTGGCAAT
CCAGCGCGAGCTTGGCATCACCCGAGGCTTCGCTTTCGACCTTCAGGCCGTTTGCAGCGGTTTCATCTATG
CCATCACCACGGCTGATCTCTATATTCGCGGCGGCATGGCCAGGCGCGTTCTGGTCATCGGTGCGGAAACC
TTCTCGCATATCCTCGACTGGACCGACCGCACCACCTGCGTTCTCTTTGGTGATGGTGCAGGCGCGATCGT
TCTCGAAGCAGCCGAAGGCCATGGCCTGACTTCGGATCGCGGCATTCTCGCCGCCAATCTGCGCTCGGATG
GCAATCATAAGGAAAAGCTCTACGTCGATGGCGGCCCGTCCACCACGCAGACGGTGGGCCATCTGCGTATG
GAAGGCCGCGAGGTCTTCAAGCACGCCGTCGGCATGATTACCGATGTGATCGAGGCTTCCTTCGAGGCAAC
GGGCCTCACGGCCGAGGATATCGACTGGTTCGTGCCGCATCAGGCCAACAAGCGAATCATCGACGCTTCGG
CAAAGAAGCTTCACATCGCTGAAGAGAAGGTCGTCATCACGGTTGACCGGCACGGAAACACCTCTGCCGCC
TCGGTTCCACTGGCCCTCGCCACCGCTGTTGCCGATGGTCGCATCAAGAAGGGCGATCTCGTTCTTTTGGA
AGCCATGGGCGGCGGATTTACCTGGGGCGCGGTTCTTGTGCGCTGG
>BRA0618
ATGGCGTCAATCGAAATCTTGATCACTCTTGATTATTCCTGTGCGATCCTGTCCCTGCACGATTTTTTGCA
GAAGACAGGTTTTCGGGGAAAA

[1] Velasco, J., Bengoechea, J. A., Brandenburg, K., Lindner, B., et al., Brucella abortus and its closest phylogenetic relative, Ochrobactrum spp., differ in outer membrane permeability and cationic peptide resistance. Infect Immun 2000, 68, 3210-3218.
[2] Ariza, J., Bosilkovski, M., Cascio, A., Colmenero, J. D., et al., Perspectives for the treatment of brucellosis in the 21st century: the Ioannina recommendations. PLoS Med 2007, 4, e317.
[3] Pappas, G., Akritidis, N., Bosilkovski, M., Tsianos, E., Brucellosis. N Engl J Med 2005, 352, 2325-2336.
[4] Moreno, E., Borowiak, D., Mayer, H., Brucella lipopolysaccharides and polysaccharides. Ann Inst Pasteur Microbiol 1987, 138, 102-105.
[5] Paulsen, I. T., Seshadri, R., Nelson, K. E., Eisen, J. A., et al., The Brucella suis genome reveals fundamental similarities between animal and plant pathogens and symbionts. Proc Natl Acad Sci USA 2002, 99, 13148-13153.
[6] Del Vecchio, V. G., Kapatral, V., Elzer, P., Patra, G., Mujer, C. V., The genome of Brucella melitensis. Vet Microbiol 2002, 90, 587-592.
[7] DelVecchio, V. G., Kapatral, V., Redkar, R. J., Patra, G., et al., The genome sequence of the facultative intracellular pathogen Brucella melitensis. Proc Natl Acad Sci USA 2002, 99, 443-448.
[8] Halling, S. M., Peterson-Burch, B. D., Bricker, B. J., Zuerner, R. L., et al., Completion of the genome sequence of Brucella abortus and comparison to the highly similar genomes of Brucella melitensis and Brucella suis. J Bacteriol 2005, 187, 2715-2726.
[9] Tsolis, R. M., Seshadri, R., Santos, R. L., Sangari, F. J., et al., Genome degradation in Brucella ovis corresponds with narrowing of its host range and tissue tropism. PLoS One 2009, 4, e5519.
[10] Wattam, A. R., Williams, K. P., Snyder, E. E., Almeida, N. F., Jr., et al., Analysis of ten Brucella genomes reveals evidence for horizontal gene transfer despite a preferred intracellular lifestyle. J Bacteriol 2009, 191, 3569-3579.
[11] Dricot, A., Rual, J. F., Lamesch, P., Bertin, N., et al., Generation of the Brucella melitensis ORFeome version 1.1. Genome Res 2004, 14, 2201-2206.
[12] Ding, X. Z., Bhattacharjee, A., Nikolich, M. P., Paulsen, I. T., et al., Cloning, expression, and purification of Brucella suis outer membrane proteins. Protein Expr Purif 2005, 40, 134-141.
[13] Ding, X. Z., Paulsen, I. T., Bhattacharjee, A. K., Nikolich, M. P., et al., A high efficiency cloning and expression system for proteomic analysis. Proteomics 2006, 6, 4038-4046.
[14] Fernandez-Prada, C. M., Zelazowska, E. B., Nikolich, M., Hadfield, T. L., et al., Interactions between Brucella melitensis and human phagocytes: bacterial surface O-Polysaccharide inhibits phagocytosis, bacterial killing, and subsequent host cell apoptosis. Infect Immun 2003, 71, 2110-2119.
[15] Stabel, T. J., Sha, Z., Mayfield, J. E., Periplasmic location of Brucella abortus Cu/Zn superoxide dismutase. Vet Microbiol 1994, 38, 307-314.
[16] Braun, P., Hu, Y., Shen, B., Halleck, A., et al., Proteome-scale purification of human proteins from bacteria. Proc Natl Acad Sci USA 2002, 99, 2654-2659.
[17] Pizza, M., Scarlato, V., Masignani, V., Giuliani, M. M., et al., Identification of vaccine candidates against serogroup B meningococcus by whole-genome sequencing. Science 2000, 287, 1816-1820.
[18] Wizemann, T. M., Heinrichs, J. H., Adamou, J. E., Erwin, A. L., et al., Use of a whole genome approach to identify vaccine molecules affording protection against Streptococcus pneumoniae infection. Infect Immun 2001, 69, 1593-1598.
[19] Labaer, J., Qiu, Q., Anumanthan, A., Mar, W., et al., The Pseudomonas aeruginosa PA01 gene collection. Genome Res 2004, 14, 2190-2200.

What is claimed is:

1. A library comprising: a set of 205 isolated, synthetic or recombinant Brucella suis surface antigenic proteins encoded by Brucella suis nucleic acid having sequences of SEQ ID NOS 1-205.

2. A library comprising: a set of isolated and purified Brucella suis surface antigen genes having sequences listed in SEQ ID NOS 1-205.

3. Recombinant constructs of Brucelle suis genes having sequences listed in SEQ ID NOS 1-205, wherein said genes are amplified and purified from recombinant cells.

4. A library comprising: a set of Brucella suis surface antigens encoded by nucleic acid having sequences listed in SEQ ID NOS 1-205, wherein said surface antigens are isolated and purified in a 96 well plate format.

5. A library comprising: a set of recombinant constructs containing sequences of Brucella suis surface antigen genes of SEQ ID NOS 1-205.

6. A set of expression vectors comprising: 205 PET101/D-TOPO vectors, each containing a different DNA sequence encoding one of the 205 different Burcella suis surface antigens.

7. The set of expression vectors of claim 6, wherein each vector further comprises a differential cloning site, a T7 lac promoter for high level IPTG-inducible protein expression, coding sequences for ampicillin resistance and coding sequences for C-terminal V5 epitope and hexahistidine (SEQ ID NO: 411) expression.

8. A vaccine candidate derived from testing a complete gene library of Brucella suis surface antigens.

9. A vaccine comprising one or more Brucella suis surface antigens selected from the group consisting of antigens encoded by DNA sequences of SEQ ID 1-205.

10. A method of detecting antigenic proteins of Brucella suis comprising:
a) creating a library of Brucella suis surface antigens;
b) diagnosing antigenicity of Brucella suis proteins by comparing a sample from a patient or immunized or infected experimental animal to said library to determine if Brucella suis antigens are recognized; and
c) diagnosing antigenicity if said antigens are recognized.

11. A method of diagnosing exposure to Brucella suis comprising:
a) creating a library of Brucella suis surface antigens;
b) comparing a sample from a patient with said library to determine if Brucella suis antigens are present in said sample; and
c) diagnosing exposure of said patient if said Brucella suis antigens are recognized.

12. A bioinformatics screen of the complete Brucella suis genome to identify genes that encode putative surface or secreted proteins comprising:
designing primers to enable PCR amplification of predicted genes with appended sequences;
arraying the primers and conducting cloning reactions in a multiwell microplate format suitable for liquid handling with multichannel devices;
cloning of the predicted genes into E. coli.;
purifying of plasmids containing desired sequence;
verifying correct insert size and sequence in the purified plasmids;
storaging the plasmids and the E. coli containing vector with cloned genes;
cloning the genes into an expression vector;
storing of expression plasmids and E. coli containing expression vector with cloned genes; (Protein bank);
inducing protein expression from protein bank; and
verifying expressed protein size.


SEQUENCE LISTING (413 sequences)

SEQ ID NO: 1 - DNA, 525 bp, Brucella suis
atgagcgaaccgaacccaaaacgcggatttcgtccgctggcggatatggcgtcgggtctggttgacccggtgctgcgcaagcgcgcgggcatcaatcttgctttgctgcaagcctgggaggatattgtcggcccggccatcggcgcgtcctcgcgaccgcttcgcattctctggccgcgtcgtattcgcgaagacgatcccttcacgcctgctacactggttattgcctgcgagggtttcgcggcactgcaaatccagcacgaaaccggtgaaatcatcagccgggtcaatggtttcctcggctttgcggccattggccgcatccgtattaagcaaaaaccgcccgtcattgcagtgaagcgccgtgtgaagcgccttgcgtcgctcggccccgccgaagagcgcagtgtggacaaggcaacggcgggcatcgaggatgatgcgctgcgccaggcacttgcccgcctcggcaggaatatcctcgctgaaaagcgcatgctgggcaaaaagggcggctcgaaaaac

SEQ ID NO: 2 - DNA, 108 bp, Brucella suis
gtgaaggggatttcgcttccacactcttttcatttgaacgctgaaattccatattgggagcatggcttcctcaaaagacaaacatatccgggattcgcgcaacaaggc

SEQ ID NO: 3 - DNA, 579 bp, Brucella suis
atgaaactgtcggcaagagcttcaatatttacattagcacttcttgtttcaacaaatgcttatgcatcatcggccatcgtcacatcgacggttaatctgcgcaccggccccggcacccaatatggaacaatcggtgcgataccgaacggcgtcggcattatggtggcgggctgcacccgcggctatggctggtgtcaggtctcctatggcggcatgaccggctgggcggcatcgcgctatatcgccattcaaaccggcaacggctacacgacgaacgacaatttcggatcgaccgccgcagcggtcggcataccgctcattgctggaatcgtcatcggctcggccatcgcaaacagcggccctgcctggaggcccggcccctatcgcggctggggtggcaggcccaacatctataatggctgcatcgggcgcaattgccagtcgaactggggaccgccgcgcccgcattggacggaccgccccggctggcgcgacaatccgggttatcatcccaatttcggtcatggtttcggtcacggctttggtcatgggccccgcatgcgtctcggcggcaggcgg

SEQ ID NO: 4 - DNA, 336 bp, Brucella suis
atgccgaaggatgccaattcagcctatgatattttcggcggctgggtgatggcacagatggatatggctgctggaatccgttctgccgagcgcgccagggggcgcacggtgacagcagcggtgcgcgaaatggcattcgcccgaccagtgaagattggtgacacgctctgtgtttatacggatataacgcgggtgggccgtacgtccatcacgctccgggttgaagcgtgggcaaagcgctatctcagcgataagcgtgacctcgtcacgcatgctgaattcatcatggttgcgctggatgcggacggcaagcctaccccggcaccgcaggaagat

SEQ ID NO: 5 - DNA, 609 bp, Brucella suis
gtgtgtcgagggcggattggccttcacaaagcgcctcgcgctatccgagcatcgcgcgaggtgaggcgggcaaggcaaactgccggtgcggccagattcctgaaaagggccggcgccgggcgaaggaaaggactgggtatggaaaagttccctatgacccctcgcggttttgaaaagctcaaggaagagctgcgctggcgtcagcaatcagagcgccctcggatcattgaggcaatcgccgaagcgcgcgcccatggtgatctgtcggaaaacgccgaatatcacgccgccaaggaagcccagagcctcaatgaagggcgcatcaacgaattggaagacctggttgcacgcgccgaggtgatcgatgtttccaagttgacgggcgacaggatcaaattcggtgcgaccgttacgatgatcgatgaagacaccgaagaagaaaaaatctaccagatcgtcggcgatcaggaagccgatgtgaaggaaggccgtatttcgatttcctctccgatcgcgcgcgcccttatcggcaagggcgaaggcgacacaattgaggtcaacgcaccgggcggctcgcgttcctacgaaatcatcgctttgaaattcgtc

SEQ ID NO: 6 - DNA, 192 bp, Brucella suis
gtgaagtggttcaacaccaccaagggcttcggcttcattcagcctgatcagggcggcacggacgtttttgtccacatctccgccgttcagcgcgctggcctcaccacgcttgatgaaggtcagaaggtctcttacgagatcgttcaggaccgccgttcgggccgttcgtctgccgacaatctcgtcgcagca

SEQ ID NO: 7 - DNA, 672 bp, Brucella suis
gtgggggccttccgccaggatgagctgttccagctcgtttgccaggcgcgttgcgaactgctcttcgctctcacccggcagcgcatcgtggtaatgatgcgggcagccggtatgaagaatacgatcgatgggcagatcgaaggagcggtgattgttgggcaggcccgtcaggctggcggaggcaatggtcacgccatgatagccgcgcttgcgcgaaatgatcttcttgcgctcaggctcgcccagcgcattggagcgataccagatgagcttgacgacggtatcattcgcttccgagccggaattggtgaaataggccttgttcatcggcaccggcgccatggcgacaagcttttcagccagatcgatcaccgggccatgcgagcgataggaaaaggtatgataaaatggcagtttcttcatctgccgcgcagcagcttccgccagacgctgttccgaaaaaccaacgccgacactccacaggcccgacatggcttcaatatagcgtttgccggcaatatcttcgacatagatgccgtcgccgcgctcaataacgagcgggccttccgcttcaagacggacagcatcggtgtaggaatgcaggtgataacggatatcgcgggcttcaagcgagttcggctgtgcagtcattcattcatcctccaagaacga

SEQ ID NO: 8 - DNA, 1668 bp, Brucella suis
atgtatcgttatgatgaattcgaccgggattttgttgcagcccgcgttgcccagttcaaggatcaggttgcgcgccgcctttcgggcgagctgacggaagaccagttcaagcccttgcggctgatgaacggcctttacctgcaattgcatgcctatatgctgcgcgtggcaatcccctatggaacgctgtccagccgccagttgcgcaagcttgggtctatcgcgcgcacttatgaccgtggctttggccatttcacgacgcgccagaacatccagtataactggcctgcgctgaaggatgttccgcagattttggcagagctggcagaggtggaaatgcattccatccagacctccggcaattgcattcgcaatgtgactgccgaccattttgcaggtgctgccgccgacgaagtgacggacccacgcccgcttgccgaaatcctgcggcaatggtcgtcgctgcacccggaattttcctatctgccgcgcaagttcaagattgcgattgtcgggtccgagcatgaccgtgcggccattcaggtgcacgatatcggcctgcaactgaagaagaacgaggcgggcgagcttggccttgccgtttatgtcggcggcgggcagggccgcacgccgatggtggccaagaaaattcgtgacttcctgcccatcgaggacatgctgacctatgtgacggcaatcgtccgtgtctataatcttcatggtcggcgtgacaacaagttcaaggcgcgcatcaagatccttgtgcatgaaaccggcatcgaacaattgacccgcgagatcgatgcggaatgggaagaaatccgccatggcgaattgaagctgccgcagcgcgatatcgatgtcattgaaagctatttccgtatgcccgacctgccgcagcgccccgaaggctgggaaattctcgccgtggcacagaaggctgatgcggaatttgcaagctgggtcgcgcgcaatgtcaccgcgcacaagcatcccgattatgccgtcgtcaccatctcgctgaagcctgtcggcggcattccgggcgatgccagtgctgaacagatggaactggtggccgatgtggcgcaaacctgttccttcgatgaaatccgggtgagccacgagcagaacctcgtgctgccacatgtggccaaggccgatctgccggcagtctacgaccgcctgcaatcgggcggactggtgacacccaatgccggtctcatcaccgatatcattgcttgccccggtcttgattattgcgcgctggccaatgcacgctccattcccgttgcgcagcgcatttccgagcgcttcgccgatctggaccgtcagcttgaaatcggcgatctcaagatcaagatttccggctgcatcaatgcctgcggccatcatcatgtcggccatatcggcattctgggcgtggagaagaagggtgaggagctttaccagatcacgcttggcggctcggcggacgaaaactcggccattggtgaaatcaccgggcgcggtttctcgtcggaagaggtggtcgatgccatcgaaaccattgtcgatacctatctgggcctgcgcgaaagtgcggaggaaactttccttgccgcttatcgccgggtgggaatggagcctttcaaacgcgctctttatggggagataagaagtgctgcc

SEQ ID NO: 9 - DNA, 1485 bp, Brucella suis
ttgcttccagcgccagcaggcgtctgctccgtgctggaagtgtttgaaaggatgatgcccgtgtctttatcgcaaacgcgacttctggctgattttcctgctttcttccgggtttcgggcgaagtggtggcggtcgtgggtggtggcgagraagccttgaacaaggctcgcctcgtggcgcagacggctgcgcgcctgcgcatcattgctgaaaaggctgagccgcatcttgccgcttttattgaaagccatggggtggagcatattgcgtcaccctttgcgccggaactgctggatggtgcgaagctcgtttttgtcgcgaccggcgatgaggccggggatcgtgccgccgctgccgccgcaagggcccgcaaaattccggtcaatgtggtggaccgtcctgccctttgcgattttctgacacctgccatcgtcaatcgtgcgccggtcacgatcgccatcggttcgcagggaaccgccccggttttggcgcagatggtgcgtgcgcgtatcgatgcagccttttcgccgcgtcttggcgagcttgcgcattttgcggaaagctggcgccctctggtcgagcgggcgcttccaaaagggcttgcgcggcgtggtttctggcgcacattcttttcgggaaaagtggcccgcgcggtggagagtggcgaccacgaggcggcgcacagggctgcggcggaactgatcgggcaggggcaggacgcttccggccatgtctggctggttggggccggtcccggtgctgaagaccttttgacgctgcgcgcccagcgcgtgctgatggaagccgatgtgatcgttcatgatgcgctggtgccggaaggcgtcatcgccatgggacgccgcgacgccgagcgcctgccggtcggcaagcgcaagggttgccattccaaaagccaggatgagatcaaccgcctgctggtgaagctggggcaggagggcaagcgcgtggtgcgcctgaaatccggcgatccgctggtctttggccgtgcgggtgaggaaatggccgccttgcgcgccgccggaataggctttgaggtggtgccgggcatcacctctgcctttgccgctgccgccgatatggaactgccgctgaccttgcgcggcgtcgcgtcctcgctcgtcttcaccacgggccatgacatgaccggcgatgtgctgcccggctgggcgaagcttgccgtctccggcgctacgattgccgtctatatgggctccacggttgccgcttccgttgcctcgcgtctgattgccgccggcctgcatgaggatacggcggttgcagtggtggaaaatgcaagccgcagcaacaagcgcatgttccatggcacgctgaaggatttgccgcagcttgaaaaccgcaaggagctttccggcccggtcatggtggtcattggcgatgccgtcgccggtgccgccatcggcagggcgcaggccctttccctgaaacctgttcccgtcgcagcc

SEQ ID NO: 10 - DNA, 1137 bp, Brucella suis
atgacgatgcaatggttctcgctcgcgcgccttcgcgccatgctgatgaaggaattcatccagatgtggcgcgatcatatcactttcgcgatgatgctgatcgtgccgctcatgcaattgctcctgttcggttttgcgatcaacaatgatcccaaaagcctgccaagcgccctcgttgcactgagcaatgatcattatacgcgcgcaatgatttcggcactggaaacgacgaactattaccgcttcgaccatgtggtgcatagtgctgccgaagccgatgcgcttctggccaatggcagcgtttcctttgtggtgacgatcccgtccgattttgcgcggcgcgtcgatgcgggcgacaggccgcagcttttgatcgaggctgacgccaccgatccatccgtggcaagcggcgcaatctctacgctcagcacggtggcaagccaggcattgctgcgcgagcagggcagacaggccgaagcggaaaacgcctcgcgcagccagctggaggtggtggtgcaccgccgttacaacccggaaggcatatcgcaatataatatcgtgccgggcctgctcggcgtgatcctgcaaatgacgatggtgatgatgacggccatggcgctgacgcgcgaaaccgagcgtggcacgatggaaaatctgttggccatgcccgccacgcctgcggaaatcatgctgggaaagatattgccctatcttgccgtgggcggggtacagatggttgtggttctggttgccgccaaacttattttcggcgtgccttttgtcgggtcgctgacgcttctcctgagcagtgtgctgattttcgtgctgtcgctcgttctgcttggctatacgatttccaccgtgtcgcgcacacagatgcaggcaatgcagcttaccttctttttctttctgccctcgctgatgctttccggcttcatgttcccgtttcgtggaatgccggactgggcccaggcgctgggcaatatttttccgctcacctatttcctgcgcatcgtccgtgcggtgatgctgaagggggcagggcttgccgatattgcgggagaggtgatggcgcttatcctgttcgtttttctctttgcgggcttggcactgttgcgcttccagcgcacactggat

SEQ ID NO: 11 - DNA, 117 bp, Brucella suis
atgtctcccaaaaagctggaaatgctcgtgaggtcagtaaccatggctttaccattgcgatgcgggaattcggcttgcacccttgaagtccggcttttcacaagccatatcagtctt

SEQ ID NO: 12 - DNA, 462 bp, Brucella suis
atggatcttagcgatctgattcagcccggggcgattatcccggcgctgaaagccagctccaaaaagcagcttttgcagattctgtctgagaaggctgcggtactgaccggtctccctgagcgtgaagtttttgaaacagttctccagcgtgagcgcctcggttccaccggggtggggaatggcgtcgccattccgcatggcaagctggcgagcatcaacaaaatcgccggaatctttgcgcgccttgaaacgccggtcgattttgaggcgctggacgatcagcctgtcgatctcgtttttctgctgttggccccggaaaatgccggtgccgatcatctgaaagcactgtcgcgcatcgcccgtttgctgcgcgaccccgatatggtggccaaattgcgcggcacccatgacgcgcaggccctctattcattcctgacggctcacccgacgtccaacgccgcc

SEQ ID NO: 13 - DNA, 654 bp, Brucella suis
atgtatatggaacgcgagacgggcaagatgaacggggacacgatggtccgcactttgcggataggattttcggttctggcgcttgccgcctttgctgcgccgggcgcttcctgggcgcagcaggccggtgatgggtccgtgcccggcctcaagctttccaacagcaaggatcccgtcaagatcgatgccgacaagctggaaatgcgcgacaaggaaggcgtggccgttttcaccggcaatgtggccgtttcgcagggcgacgccttgctgaaggctggccagatgaccgtttattacagcaaggccaagaagggcgctgaagcgcccgaacctgcaaatgcaggcgtgggcgggattggttcgtcgagcattgaccacatcgatattgccggcaaggtttatctcaagtccggcacgcaggtcgcgaccggcgacacaggccgctatgacgcgaagaaccagatcatggttctccagggccagaaggtcgtgctgacggacggtgacaatatcgtgaccggctgcaagctgaccgcccatctcgatacggggcgggccaatgtagaaagctgcaagggccagaccaaggggcgcgtgtcgatcatcatggctccgaaggatcaacagcagggcggtgctgcgcccaagcggaac

SEQ ID NO: 14 - DNA, 1386 bp, Brucella suis
ttggtcagaatgcgggggcaggcaattgcgataaactggcagcggtatatctcatgcccccgatataccggaaccaggcttcgagggagcttctcccccgaagtccggttcccggaaccgtcacggggcaagcccggtgtccagctaaagagccttgccatgaaatgtgcctacttcgtgcgcccccatattggcggaacctattcggttttcacacggttgcgtgcttcgcttgcccggtctggcatggatctgcgctggcttgcagccggtgcgccgggcgaagcggtggccgtgccgcgcgagcaggggctggaagatgccatgcgccggggcgacatcatagatcgcatgggcgttctggatgaagaaacccgcgccaaacgctttgtcgattttcttcgcgaaaacaggtttgaagcagtttttgtgaatgtgctttctcaccgtttcgagaccaatctcgtgcgctatcttcccgcggatattctgcgtgtgatgatcgttcacagcatcacgcccggcacctatgcggcggcgcaggcgatccgcgatcatgttcacgcgacggtcggtgtttccaaacggtgccgcgacgatctggttcgtggctatggcttcgaccccagccgcacactcgtcattcccaatgggctgacgcccgatccgcatgtccggtgcgcagcgccggagattgaaggcggacggctgcgactgctctatctggggcgcatcgatgacagttccaagggggtcttctggctgcccgcgatcctgcggcggctgcaatgcgattatcatctgacggtggcgggcgacgggccggacctcgcagccttgcgcatcaagctggctccctttggcaacaggatcagttttaccggctgggtcgcgccatccgatgtttcatggctggtgagccagcatgacgtgatggtcatgccatcgcgctatgaaggtttcggctcgacgctgattgaggcaatgagccagggatgcccggtggtggcgtcgaggataccgggcgtgacggatactatcgtcaccgatggtgaagacggttttctgtttccgatcggcaattgcaggcaggcggcgcagcagatcgagaggcttgccgccgatccgcgcctgcgcgtggcgatgggtgcggcaggcgtgcgcaaggtgatcgcggaattcgacagcgaaatcgtcgggcgcgcctatgcgggattgctccagcatctgcgtgaaaatcgcccgcccattacgccgcctctggctctttcgcagtggtccatgcccaatgccctgcacaaaaacctgcgcagccgcttgccgcagccggtcaagaactggctgcgccgcgtgcgggagcgcgtgcacacggtatggtcgacggcc

SEQ ID NO: 15 - DNA, 474 bp, Brucella suis
atgcacaagccgaaactgcttctgctggacgagccaaccgcgggtgtggaccccaaggcgcggcgtgatttctgggatgaaattcatcggctggcagatggcggcctcaccgttctggtttccacccattatatggatgaggcggagcggtgccatcgcatcaactatatttcctatggcaagctgcttgcaacgggcacggtcagtgaagtgatcgaacgcacgggcctcaccactttcttcgtcagtggaaagcggctcggcaatgtggcgcgagagcttgaagaaatgccgggcgtcgatcaggtcgcgcctttcggggctacattgcatgtggtcggctctgacagggagaagctgacggccgcactcgacaggatcaagcatcgcgaagacatccatgtagaggcaggccagaccagccttgaggatgttttcatccagttcatggccggggcgaaggacaatatgcaa

SEQ ID NO: 16 - DNA, 1290 bp, Brucella suis
atgactgaaacaggtttcagtttctcaagcggcttcataggccgttcttggttgaaaattcaagaaaattgcgacttcagatatctcgaaggcgttgttaaggagataggtaaggtggtcgggagcggcaagaggctctatctggattataatgccagtgcgcctttgcttgatgaagcgcgcgatgcgatcatcgcggcattgggcattacgggcaatccatcctccgttcaccgcgaggggcgggctgcccgtgcgctggtcgaatccgcccgccgcagtgttgcgaccctcgttaatgccaggccggagcatgtcttttttacgtccggtgcgacagaggcggcctcgacgctgctgacccccaattacatgatggggcgttcgccggtgcgtctctcacacctttatgtcagcgccaccgaacacccatgcatgatcgccggcggacagttttcgcccgatgacatcaccgttcttccggttgatgccaatggcatcctgcgcctggacattttgcgcggcctgcttgccgcccatgacaggagcaagggtcttccgctggttgcagtacaggcagccaataatgaaacgggcgtgatccagccggtggcggagatcgcagccatcgtaaaggctgcgggcggcatttacatcgtcgatgccgtgcaggctgcagggagaattaaattagatattacagataattgcggtgattatctcataatttcctcacacaagatcggtggcccgaagggcgtcggcgcggtcattgcgatctccgacctcatgatgccgcgggcgctggtgcgcggcggcgggcaggaaaaaggccaccgggcgggaacagaggcgctgcccctgattgccggttttggtgcggctgccgatgtggcaaaagcgcgaatcgacggcgaaggctggtcatccctcatgcgggatcgtctggagcgtggccttgccgaaattgcgccagcggcgatcgttcatggcagtgctgtgccgcgcttgccgaatacgactttcttttccctgcaaggccagaaggctgaaacggtgcagatcgcttttgatcttgcaggtattgcgctttcggccgggtcggcctgttcttcgggcaaggtggggccgagccatgttcttgctgccatggggcaggaagacgggccgggagccatccgtgtttcgcttgcaacggatgcgggcgaggaaacagtggatcgatttcttgaagccttgcgcaaaatcgtcgagcgtcaccagcgcacgaagcacgaattgggcgcggtt

SEQ ID NO: 17 - DNA, 2751 bp, Brucella suis
ttgccgtttcactggcaggcccggtttcagccttgtcctgaactttctgttccatatcggtcatccgaaaaacttccttgccttttccaaggcttcagccaatgcgtctacttcggcgcgggtattataaagggcaaaggatgcacggcatgtagaggtgacgccaaagcgtttcaagagcggttgcgcacaatgcgttcccgcgcgcaccgcgacccccgcccgatcgatcaccatcgacacgtcatgggcatgaatgccttcaagcgcgaaggagatgattgcgcccttgtccggcgcattgccgaaaatgcgcaagctgttgatccggccgagccgttcatgcgcataatcacgcaagtctgcctcgtgagccaggatcgcatgacgcccgatcttttccatatattcaagcgccgcacccagaccaatcgcctgaacgataggcggcgtgcctgcttcaaagcggtgtggcggatgattgtaggtgacgttttcctccgtcacttcctcgatcatttcgcccccgccctggaacgggcgcatcttttccagcatttgcgcgcgtccataaagcacgccgatgcccgaagggccatagactttgtggccggtaaagacataccagtcacagcccagatcctgcacatcgaccggcaggtgcactgcaccctgactgccatcgaccagaaccggaatgccgcgtgcatgggcaagctccacgatcttcttgatcgggaccaccgtgccgagtgtattcgacatatgcgtgatggcaacgagtttggtacgttcgctcaggcgcttctcgaactcttcgatgtggaaaacaccgttatcgtcgactggtgtgaaaaccagtttcgcgccctggcgttcgcgtatgaaatgccacggcacgatgttggaatgatgctccatgatcgagagcaggatttcatcgccctcgccgatgaacggcattccatagccataagcgacggtattgatcgcctctgtcgcattcttggtgaagacgatctcgtccacagaaccggcattgagaaaacgtcgcacggtttcacgcgacttctcataagcgtcggtggcagcgttcgagagaaaatgcaaaccgcgatgaacattggcatattcattcgcgtaagcatgagttaccgcgtcgataacgctttgcggcttctgtgcggaagcgccgttatccagataaacgagcgttttgccgtgcacctgccgcgacaaaatcgggaaatccctgcgtatggcctcgacatcataggccatggtgagcggattcttctgatccatgatcgttccttctgccttcccggcaggaaaggaggaagcgggaaactcccgccccgtcctgttcaatcaggcccaattcactcaagcatgaacggcgagccagttggaaagaaccccttcgagtgcttcaaccagcgtttcgtcttccagctcctcgatgatctcggcgatgaacgccttgatcagcaggccgcgcgcttcattttcggggatgccacgggccatcagataaaagagatagtccctggacagttccgcaaccgtcgcgccatggccgcaggccacatcgtccgcgaagatttcaagttcaggcttggcatcaaactcgccttcatccgaaagcagaagtgtgttgcaggccatgcgggcatcagtcttctgggcgatctgggcgacacggatcatgccctggaaaacgccgcgtgcacggtctttcaccacattgcgcacaatctcggtggagcgggtattctccaccaaatggcccacggtcatggtgacgtccgtatggctgtcatcggccagaagattgaggccgcgaagctcaaaatcggaatcctcgccctcgacattcacatgcacttcctgacgaaccagcttgccgcccgcattgacgatgtaaagcgtcagcctggaccccttgccgattgtcacattgaactgcgagagctgtgttgcctggccgaaatcgcgcagaatcacccagatgatctgcgcaccatcgccgaccgtcacatggttgacggatgtggagaaggcatccccctcaccgccattctggcgttcgatgatcgtgcccttgacgttggcgccgatgcgaaccggaaaccgcgtatgcccctgcccggcattctggatgttctgtatttcgagcggctcttccagttccgtgtcatcggcaatcgaaaccatccagccgtcgctgacataggcagcgttgatctggccgatcgtatcgtcgaagccacgcgacctaagctgcgagacaagcgaaccatcggcaagcgcctcacgaaccggcgtcagcgaaacgcctttgggggccttggcggcaagcgcgacaccattgctggcggcaatgatggacgaaccggcaatcagcgccggcagagcctgtgttccggcagatgtgtcgaatgccgcaacgcccttcagcaacgtgcggaaatcggtgtaatgccagctttcgatgcggcgcgagggaagcccctgaaccttgagggcttcaagggccgtgtcgcgtgcgatcacgacatcgccattgccgggcagatcgcccatgcgctcggcaaaactgtcgatcagcacgctttcagccggggtacgcggcttgggtgccgaagtggcctggatattcatgatccaaccctcctcaggcggcttcgccgattacgtcggcataaccattttcttccagatgcagcgcgaggctcttgtcgccggact

SEQ ID NO: 18 - DNA, 1275 bp, Brucella suis
atgaatatccaggccacttcggcacccaagccgcgtaccccggctgaaagcgtgctgatcgacagttttgccgagcgcatgggcgatctgcccggcaatggcgatgtcgtgatcgcacgcgacacggcccttgaagccctcaaggttcaggggcttccctcgcgccgcatcgaaagctggcattacaccgatttccgcacgttgctgaagggcgttgcggcattcgacacatctgccggaacacaggctctgccggcgctgattgccggttcgtccatcattgccgccagcaatggtgtcgcgcttgccgccaaggcccccaaaggcgtttcgctgacgccggttcgtgaggcgcttgccgatggttcgcttgtctcgcagcttaggtcgcgtggcttcgacgatacgatcggccagatcaacgctgcctatgtcagcgacggctggatggtttcgattgccgatgacacggaactggaagagccgctcgaaatacagaacatccagaatgccgggcaggggcatacgcggtttccggttcgcatcggcgccaacgtcaagggcacgatcatcgaacgccagaatggcggtgagggggatgccttctccacatccgtcaaccatgtgacggtcggcgatggtgcgcagatcatctgggtgattctgcgcgatttcggccaggcaacacagctctcgcagttcaatgtgacaatcggcaaggggtccaggctgacgctttacatcgtcaatgcgggcggcaagctggttcgtcaggaagtgcatgtgaatgtcgagggcgaggattccgattttgagcttcgcggcctcaatcttctggccgatgacagccatacggacgtcaccatgaccgtgggccatttggtggagaatacccgctccaccgagattgtgcgcaatgtggtgaaagaccgtgcacgcggcgttttccagggcatgatccgtgtcgcccagatcgcccagaagactgatgcccgcatggcctgcaacacacttctgctttcggatgaaggcgagtttgatgccaagcctgaacttgaaatcttcgcggacgatgtggcctgcggccatggcgcgacggttgcggaactgtccagggactatctcttttatctgatggcccgtggcatccccgaaaatgaagcgcgcggcctgctgatcaaggcgttcatcgccgagatcatcgaggagctggaagacgaaacgctggttgaagcactcgaaggggttctttccaactggctcgccgttcatgct

SEQ ID NO: 19 - DNA, 150 bp, Brucella suis
atgcatgaggaaaagctgcccacgctcgaaagcggcgaaagcatcttctctcatgtgctggatatcgccaatggctatgtttcgctcatccccgcccatttcgcggccgccccggatgcggaacccgcagtgtcgcttatcgcagccgaa

SEQ ID NO: 20 - DNA, 840 bp, Brucella suis
atgaaatcctgtctgttgagcctgacgataagcatgtgccttttcgctcctgccgcccatgcggcgggggaagaaccggctgcaccgcatttcttcaaccagaaagagcgcctgcccctgccttcgctgcaagggctcaatcgtctgcgcttcatcaccaccgtcgattttccgccattcaacaggcttaacgaacgcgggcagctttccggctacaacattgatctggccaaggcgctttgccagcagctcaagatcgaggacatctgccagatcgaggcggtgccgtgggcggaactggaagagcgtgtgctggatggacaggcggacgcgatcgttgcggggtgggaaccgaccgacaggaaccgggaaaaattcaccttcacccgcagctatatgcgcctgcccgcacgctttgcgacctccaatgccaaaagctttacagaacccgctgcaaaggcgacgcaggacaagcccgtcggcgtgattgccgggacggcgcatgaagcgcttttgaagaactatttcccacaggcaaaacccgtgccctaccccaatcgcgatgccatgctgaccgcattgaaggacggcaagatcgacacggttttcgatgacggcatgtccctctcggcctggctcgacagcgcagaaggcagcgcctgttgtactttcacggacgggccctatctggcgcctcaatatctgggttccggcctcagcatcgctgttacccggcaaaattccgccctggcaagcgccttcaacaatgccctgcaagccctgcaacaagaaggtaagctgaccgagctttacctgagatatttcccggcaagcttttat

SEQ ID NO: 21 - DNA, 756 bp, Brucella suis
ttgtcgatcctgcgctttgctgccgggtttgccagtgccgtcaccatgatcctgtgcacggccatcgtgctcagccatatcacgcgcgcacaaagggcggggcttggcgcgatgcattttggcggtgtcggcagtggcatcacgctgtctgcgctgctggtggccctgatccgcttttcggggcttggctggcgggccgactggattggcgctggcctccttacccttgttatcatggctgtggttgccgctttcgtgaaggaggggccggtcggcggcaataatggcatgcgtgagccgcccctgcccaacagcttcgcctttcatgcgctggcgtttgcctatggcattttcggctttggctatgtcatcacggcgacattcctcatagccatcgtgcgcgccaatgacggcgggccggtaatggaggcggctgtgtgggtggtaacgggcctggcagcagcaccttccatctggctgtgggcacctgtgggacgcaaattcgggttattttccgctttcgcgctcaccgctttcggcctgcaggccgcgcgcattcttgcgcctgcttcacccaaacgcgcattggcgcgcatgacggtctgcttcagtatcgggcagatcatagggccgctcgtggcgggcttcatggcgcagcattcaggctcgttcacatctgcaagccttgctgcggccttgtctttgatcgttgcggctgctatcggcttttcagccgggcgcgcagcccggctgcat

SEQ ID NO: 22 - DNA, 1425 bp, Brucella suis
atgaaatatccgcgcaatctcgtcggctatggacgcaacacgccagacccgcgctggccgggaggggcaaatatcgccgtgcagtttgtcgtcaattatgaggaaggcggcgaatgctgcattctcgatggcgacccggcttccgaatgcctgctttccgagattgtcggcgcgcagccctggcggggccagcgcaatctcaacatggaatccatttatgaatatggcgcccgttccggcttctggcgcctgtggcgcgcctttacccgccgcaatatgcccgtcaccgtctatggggtgacgcttgccatggcgcgcaacccggaagccgtggccgccatgaaagaagccggctgggaaatcgccagccatggcctgcgatggtgggaatacaaagacgtgccggaggagatcgagcgcgaacatatccgcgaggccgtgcgcctccacacggaagttaccggctcgcgcccgctcggcatctatcagggcaagccctccgacaacacgctgaagctggtgatggaggaaggcggcttcctctattccgccgattcctatgccgacgaactgccctattgggtaaagggcccgaaaggcccgcacctgatcgtgccctatacgctggacgcaaacgacatgcgtttcgccacgccacaaggctttaattccggcgaccagttcttcacctatctgaaagacagtttcgacgttctctatcgcgagggtgaggaaggcgcgcccaagatgatgtccatcggcctgcattgccgtctggcggggcgccccggccgcatggcggcgctggaacgtttcctcgattacgtgcagagccacgacaaagtctgggtggccaaacgtgtggatatcgcccgccactggcataaacaccaccagccggtcgtggccgatgtggtgccttccaaagcggacaggcaggcctttgtcgcgcgttatggcagcattttcgagcattcgccctggattgccgagcgcgcctatgatgcccggctcactgtggaagaagacagtgcttcgggccttgccgccgcgatgaccaaggtgttccgcaccgcagcgccggatgaacggctggccgttttgaaagcgcatcccgaccttgcgggcaagctggcgcaggccaggcgactgacggcggaatcaaccgccgaacaggcatcggccgggctggacgcgctgacggacaccgaaaagcaggcctttaccgaattgaacgacgcctataccgccaagttcggctttcccttcatcattgcggtcaagggccgcaacaagcaggagatactcgacgccttcaggcggcgcgtttccaacgaccgtgatacggagtttgaaaccgcctgcgcgcaggtggaacgcatcgcgctcctgcgcctgaaagacatcctccccgaaacactctat

SEQ ID NO: 23 - DNA, 447 bp, Brucella suis
atgctggagaaaagcctccagcaatgctcattcgcgcgcggtaaggcaaagtgcgcgcccgtcgcgcatgacggacttggctgcaaggtcgatctgcaagccgccgatttcgatcaggggattgggattgccgctatagcgccgcgctaccgcgttcagccgtgcggaaagatcgaagggcttcgtcagataatcgtcggcgcctgcgttcagtccctgaatgcggtcggaaatctggtccagggccgtcaatatgatgacaggcgtggaacagccctttgtgcgcagatcacgcaagaaggacaggccttgcccgtccggcagcatcaaatccagaagaacgagatcgtagcgcgcgctctccatgtagtcatgggcggtgtcgagacggctgacccaatcaacgcaatggccttcagccgcgatgtggtcgcgcacggcagcgcc

SEQ ID NO: 24 - DNA, 861 bp, Brucella suis
atgcgctcgtgcggggcagggcaagcacggccctttggcccacacagcttttcctcccacattcctgtcacagctcgtatttcggcggcggagcatttgtattgccacttttgcgttcctatgtgcgaaaggacaaaaccagacaggagatttcggatgttcgatgccaaaaaatttctcgatcagtttctcggatcacaggtgccgggtatgtcgggaagtgtccgcgacaaggctggacaggtaacagatatggcgaagaagaacccgcttgcgacgggtgccatagccgcagccatttttggcacgaaaacgggccgcaaacttgctggcaatgtagcaactgtcggcggcatcgccgccatagccggattgggctatcttgcctacaagaattataagtccggtcaggccccgcaggaggcggaacagccggtggcaaaagagccggagcttcttccggcgccaccggccgattcacccttccatccgcattctcccgcgatgagcaatagtttcgcgctgacgctggttcaggccatgatcgccgccgccagggctgatggttatattgatgatgccgaacgtgcccgcatcatggaaaaggtgaagatttccggtctggacgacgaggctgaggccttccttgcaagggaactggccgaacccgtcgatctcgatgtgctggttgccgcagcgcagacggaagagcagaaagttgaactttatacggcatcgcggctggctatcgatccagataaccgcgccgaacgcggctatctggatatgcttgccgggcggcttggcctgcaagatgcgctggtggaccacatcgaagcgaccgttgcttcagtgaaagtc

SEQ ID NO: 25 - DNA, 189 bp, Brucella suis
gtgctggcccgggccatattcacgacggttgaccggggacttcggacggccccagatgttttcgccaagacggcggtcaatcttatactttgcggattcgcgcttgctcatcgcatttccttcaaaaagttagagcgcccggaggcgctggaggaaacgcgccctcctctgccctgcgtttccgaggac

SEQ ID NO: 26 - DNA, 126 bp, Brucella suis
gtgaaattcaatcgtccgcacgcgctgattcaaaactatatcaatgaagtatggcaaagtgcggcaatgaagatgcgaacgaacgatccgccgaagcccctgcgcgatgaaaatagtttcaccgat

SEQ ID NO: 27 - DNA, 231 bp, Brucella suis
atgtgctcaaagggcaagaagcttcaccaaaatatccgcgcccgccggcaagggatgaaagccgtgaagctttacccccgccgatcaaagatgacgctggaacgcgccctgtcgtggattcaggatgacgaactggtggaagtgacgccgaagtcgatccgtcttcgcaagctttatctcgatcccaatgagcacaagcgttttcagaagagcaagtcgtcgagtgctgca

SEQ ID NO: 28 - DNA, 993 bp, Brucella suis
atgtcggaactgctttttgaaaccgatgcaaatccgatcccgagccgaataagaggcggtcttttccatgcgccggacgggaaggccctgcgctacgccctgctgaaagccgaaagccggccgtgccggggcacggtcatcgtgcttcagggccgcaacgaattcatcgaaaattattatgaaaccatgtccgacctcgccgggcgcggttttaccgttgccaccttcgactggcgcggccagggcggctcgcatcgcctgctgcgcgaccgcctgcgcggctatgtgcgcagcttcaacgattatgccgacgatctcgaccatttcctgaccggaatcgtgctgcccgactgcccgccgccatttttcattctcgctcactcggcaggtgcgctggtcgcgctttcctcactggaacggctctcctcccgcatcacccgcatggtgctttgcgcgcccctgatggggcttggcgggcagaaaatcagcgatgacaatgtgcgccgcattaccgccgcgctgcgctggatcggcctggggagaatctatgcggcaggcgggcgcaccctgtcggcggcgcgcgcctttgccgacaatccactgaccagtgatccgttacggttcatgcgcaatgtggaaatcacacggacttatacggacctcgcgcttggcggccccaccgtgcgctgggtctggagcgcactggaaaccgcatggcgcatcaaccagccggatttttacaaaagccccatcgcgccggttctgattatcgcggctggtgctgaccgcgttgtttcaacagcggtgatcgagcgtttcgtggcgcgcacgcgaaacatctcgctcgccgttattgacggtgcgcgccacgaaatgctccaggaagcagatttctaccgcgaacaggtgctggcggctttcgacgccttcatccccggctcatcgccggtggaatccatgccccaaagtcttgaacccgacctctctcaaatc

SEQ ID NO: 29 - DNA, 822 bp, Brucella suis
atgtttcaaaagttttcagcggcgggtgccgcgctattattcaccgtcggcattgcctgtgccaatgacatggcatcaaaggagcatggctctgaaacggtcacgattgtcgagccgcaactgcatattaagccgggcggcgcggcggcaccgcaagtggcgctgacgcttgatgcctgcatgggcaagacagatcatcgcattctggacgtgctggtgaaagaccgcattccggctacgattttcgtgacagggcgatggataaagcaaaatgccgaagccgtggcggttctcaaggctcatcccgatctcttcgatgtagagaaccatggcgcgatgcatgtgccagccatcacgaacatgccgctgatgtatggcatcaagaccgctggctctctcgatgccgtgcgcaaggaaatcgaaggcggtgcggaagcgattaccgccgagggcatgccaaagccgctttgatatcgcgatgcaacggcgcgctattccaccgatgcggtgcagcttgccaccagcatgggctacaagatcgccggttattcgctgaacggtgacatgggcgcgagcttgcccgcagagactgtggaacggcgcatcgccggtgcgcatgacggcgatgtcattatttcgcatatcaaccagccgacccgttcttcgggcgagggggtagcgaaaggcattctggctttgaaggccaagggcatgaaattcgtgcgcctgaaggatgtgcaaaccacgatggaactcaaccccgtcccggaacacgatcttcctgtcaacaccgctgcccagaaaaaacaggaaacggtgaaa

SEQ ID NO: 30 - DNA, 1002 bp, Brucella suis
atgcgaaggcgcggcggtgtggttgcgctcacgcaccacctgatagcccggacgccagatataacgcaccggcgcactcagcatatgcacaagacgggtgaacgggaacaggatgaagatcgtgagcccgaggaagatgtgcatcttgaacatgatggaagcatcggcaacatagctggaggcggccgggttgaaggtgaagatgccctgcgcccagttcatgaacttgaccatttcgccgccatcgatgtgctggagcgaaaccgggatcgaactcaggccgagcagaagctgcacccacaacaagatgatgatgagtgtgtcgcttgggcgcgaggaagcgcgcacgcgcgggtcgaacaggcggcgatgcaccagcatgctggcgccgatgattgccatcaggccggcaatgccgcccgcgacgatggcgaggatctgctttgcctcatggctgatgccgagcgtgtcgaagatccagatcggggtcagaaggcccacaaagtggcctgcgaaaatgacgagaacgccgagatggaacaggaccgagccccacatgagctgcctgcggcgcagaagctggctggagccggagcgccaggtatagggttcgcggtcgtagcggacgatcgagccgagtgcgaggacgctcagcgcgatatagggatagatgccgaacagaaacgtgttgaaatagctggccatcttccgtctcctagatttgtgttgatggcgaagcgggcttgcggtgtgcggcgcgcatctgcatgcgcagccgatccggcccgcaggcattttcgcctgcattggcgccgaaagtcacagcctcttcttcccagatgcggtcgagtgcggcgaggtcgtccgggtcgtcttcttcctggcgaagcaattctccgacgagtttctcgtccggcttggcattggcaagatgaagaagcgcggcgaaggccccggcatagggcgactggcgcttgcgcaggcgctcgccgattgccgccg

SEQ ID NO: 31 - DNA, 1107 bp, Brucella suis
gtgcaattatgcgacccggcgcatatgccggacgtggattttgttgatctcagcgcctttcccgatccgcgggcagaggctgggcggcgtatggaaaccgatctgtccgcaccgcttgatctgcgccgggacaggatgtccgcccatgcgctttatcgcatcggcgcgcgccaatatatctggtatatccgcgcccatcacatcattatcgacggttacgggctggcgctgatcgaacagcgttgcggccagctttacagccatttttgcggccagggcgaaagtggccaccccttccatccttttgcaagcttcattcatgaggaggacgcctatcgggcaagcccccgctgggaggccgaccgcaaattctggcaggattatctggacgggcctgtgcgccttcccatcctcgatcggggcgcggaagattatggcgaggcgggctatcatcatgacgtcgcctttcgcgacgatatttccataggccttcgcgcgatggcaaaagccacggatatcggctggcccgatcttctggtgcttttatcggggctctatctctcgcggatgcttccgatgcagggagaggacgaggggctgacgctgtggctgcctttcatgagccgctggggcagcgtgggcgcacatatgccggggcttctggtcaatatcctgccgttccatcttgaattcgattccggcgaaacattgttctctgcgttacggcgcaatatggacatgctgaaaaagcaccggctgcacgggcgctatcgcatcgaacagatttccgccgaccgcggcctcgtcagtggaagccgctttttcttctcgccgctgattaatgtgctgccattcagcacgccgcaatttgcaggttgccgggttgcgcgtgaggttctggccaatggccccggcgacggcttcaacctgacctatcgtgccgatgtcgatggcgctggccttaccttgaaaatcgaagcggcatcgaacatgatggatgcgcaaacctttgcccgcattgcggcgggccttccggcctttctcaaggcaagccttgcgccccatctcatcgagcgtccggtgagcgatcttcgccgccgtttgcca

SEQ ID NO: 32 - DNA, 1005 bp, Brucella suis
ttgaaagaaaccatcatgtcgatcacccgacgccgtgtccttgtcggtggcgctgtcgccgcaacgttttccatgcttccccggttcgcctttgcgcaaggcgagcagcaaccggtcatctggcaggggcaggccttgggcgctccggccaagatcatcttctatcatcccggccgcaaggtggcggagcggcttgtcggtgaaagcgcgcgggaggcggaaaggcttgagaatatcttcagcctctatcgcccggattccgaaattgtgcggctgaaccatacgggcgcattggcttcgcctgcgccggaaatggtggaattgcttggcatttgccgcacctgctggcaactcagcgacgggctgttcgatccaactgttcagcccttgtggcaatgcctgagaaagcacttctcgcaggcaaatcccgcacctgaaggcccttcgcgggcggagtgggatgcggcgcttgcaaagataggcttcgatcatgtgctcttcaacgcggagcgcatcgcctttaccaagcccggaatggcgctgacactgaacggcattgcgcagggctatgtgactgatcgcgtcgtgggccttctgcgagatgcaggcgtgagcgaggcacttgtcgatatgggcgaatatcgcgctatcggcgggcagccggatggcaagccctggcgtatcggcatcgccgatcttgaggcgggggccgcgccggaagaatttatcgacatcatggatcaggcgcttgccacctccagctttgcgggtttccagtttgaggaaagcgggcgtttcaaccatctgctcaatccgaaaaccggctattcggcggccctttaccggcgtgtgacggtggttgcgccagatacggcgatggccgatgcgcttgctacggctttcagcctgatggaaccggcacaaataaaggaaacgcttaaaaaactgcccggcattttcgcttataccgtcagccgcgatggcgaaaagcgccgctttggggcc

SEQ ID NO: 33 - DNA, 1107 bp, Brucella suis
atgcgagttcttcatttttttaagacctattggccagatacatttggcggcattgagcgaaccattcatgcgatcgcaaaaggtgtggccgaacacggtatcgcgtcggacgtgttatccttgagtgcaaagccggaagaaaatacccgaaattttgatggtcacatggcatataaggccaagctcgaccttgaatttgcatcaacaggattgtcgcgcgatgtgttttcccggtttcgggagttgagcagccaagctgatgtcattcattatcatttcccctggcccatgagcgatatcgtgcagatcggtgtgcgcccggataagccgactatcgtgacctatcattcggatatcgttaagcagaaactacttcttcagttctatcgccccttgatgaaccgctttctcggcagcgttgaccggatcgttgccacctctccgaattatcttgcgacaagcgatgtcctgcagcggttcagcgataaaacgacagtcattccacttggcttggacgagtctgattattctgctgccgacgccacaaccaaggcgcgctggcgcgagcggtttccgcgccctttctttctcttcgtcggcgtgctgcgttattataaagggctaagaacgctactggccgcagcaagaacttccgatattgatattgtcattgtcggggatgggcctatgaaacctcagctcatcgcgtatgcgaatgagcataatctgtcgcacgttcatttcgccggcgcgctgccggatacggacaaaactgcgcttctggagttgagtgctgggctgatttttccgtcgcatctgcgctcggaagcgtttgggctatcgctggtggaggcctctatgtttggaaagccgatgatctcgtgtgagatcggtaccggcacgagttatgtcaatctgaatgggcaaacgggaatcgtggtgccgcctcagaacccggaagcgctctcagctgcaatgcgaagcattgcacacgatacggagcaggccaaagtttttggaatgaacgcacgggcgcgatatatagagcatttcactgctgataaaatggcgttagaatatgcgaaaaattatacgtcgctgatgcag

SEQ ID NO: 34 - DNA, 210 bp, Brucella suis
atgcgcctgcaccggcaacgcacgcatggccacatgtcctgcgcacctgcttcattcgataatccagctgcacgcgaactcctcccaaagctctacgtcaggaagcttatctttcgcgttgaggtacgtaaagcaaaaatattagggcggacgcaaagagtagcccgcctgaaaatcgcaccatttcacaaaatttatggcgggattttt

SEQ ID NO: 35 - DNA, 105 bp, Brucella suis
ttgccctatcgcacaggcttcgctatccttcaagcaattgcggataaaaaggctggtattgtcacgttactggcgcaaagtgacaacaccttcgcttcaaccgat

SEQ ID NO: 36 - DNA, 2154 bp, Brucella suis
atgcgtaatacggcagaaaaagatcgcagaaacgacaagcgtaaacccttccgggtttcgcgcctgatcggactcgacgcctggattgattccgcgctctacaatttacatttccgcttgggcgaatggtgggaaaacatcacgattttctcccggcgttttcgcgtgcgcggctttcgccgtttcacggtggaagtgctggacgaaggctttacgctcggcgtcgccggttccgtgctgatgttgatgctggcgctgccagcctttgaggaaaccaagaaggactggcgcgcgcaggacgattatgccgtcacctttcttgaccgttatggcaacgagattggccggcgcggtattctgcaccggcaggcggtgcctatcgatgaattgccggatcatgtcatcaaggctgtgctcggcaccgaggaccgtcgcttcttcgaccattacggcattgatctcatggggcttagccgagcgttaagccagaacatgcgcgccaacggcgtggttcagggcggctcgaccatcacgcagcagcttgccaaaaacctgtttctttccaatgagcgcaccatcgaacgcaaggtcaaggaagcctttctcgcgctctggcttgaaagcaatctcagcaagaagaaaatcctccagctttacctcgaccgtgcctatatgggcggcggcactttcggcattgcagccgcgtccgagttttatttcggcaagaatgtaaaggacatctcgctggccgaggcagccatgctggccggcctgttcaaggctccggcgaaattcgcgccgcacgtcaacctgcctgcagctcgcgcccgcgccaatgtcgttctctccaacatggtggaaagcggctttctcagcgaaggtcaggttgccgtcgcgcgccgccacccggccagcgtcatagatcgcgccaaggatgaaagccccgactatttcctggactgggccttcgacgaggtgaaaaaggttgcagacaggttcaaccagcatacgctgatcgtgcgcaccacgcttgaccgcaatatccagaaagccgccgaagaatcgcttgaatttcaccttcgccaatatggcaaggaatataacgtatcggaggcggccaccgtcgtgcttgccaatgacggttccgtgcgcgcgctcgttggcgggcgcgactatggcgaaagccagttcaaccgcgcaaccagggcgctgcggcaggccggttcatccttcaagccctatgtctatgcggctgccatggaaaaggggctgacaccaagcaccatcgtttccgatgcaccgatcagctggggcaactggtcgccgcgcaattacgggcgcagttttgctggccgggttgatctcaccaccgcgctcgtccgctcgctcaacagcgtgccggtgcgccttgcccgcgattatctgaccacggcgcccgttgtcgcgctcacaaaggcaatgggtgtggaatcgcccatctcctcccataagacgatggtgctcggcacatccgaaatgaccgtcatggatcaggcaacaggctttaacgtcttcgccaatgcgggcatggccggaaaccgtcacgctttcacgcaaatcctggcatccgacggaaaggttctttgggattttggccgtgacgcaccaaagccgcaccgcgccctttcggaaaaggccgcacttgagatgaactccatgctggtgcaggtgcccgagcgcggcacgggccggcgcgccgcacttacgatgacgcgcgtggcaggcaaaacgggtacgacccagaactatcgcgatgcctggttcgtgggctttaccggaaatttcaccgccgccgtctggttcggcaacgataatttcacgcccatgaaggaactgaccggcggtgttctgccagccatggcctggcagcgcatgatggcatacgcccatcagaatatcgaattgaagccgctgccgggcgtcaacccgcccttccccgcccagccgaagaacccgcccgcacaggtggccgatacgaggccgcacgaaacaatggctgccccaccacgggttttatcgccgctggcgacaaaaattctcaaggaactgcatgaccgcttccttgccgcgccgcctctgcctacgatagccgagcgcacaaaagtttcggtactc

SEQ ID NO: 37 - DNA, 603 bp, Brucella suis
atgacggatcagggggagaacgagaagaagcagcggcgctccaatgcgacgatcgccgtcgcgtgcctgtccttcttcgtctgcatgatcggtgcggcctatgcttccgtgccgctctaccgcatcttctgtcaggtgacaggctatggcggcaccacgcagcgtgtggagcaatattccgacaccattctcgacaagacgatcaaagtgcgtttcgatgccaacatcgcaaatggcctaccatgggatttcaagcccatgcagcgcgaggtgacggtgcgtatcggcgaaaccacgatgataaaatatgaggcgcataatctgttcggcgaggaaacctatggccgcgcaagcttcaatgtcgcgccgggccgggccggggcctatttcaacaaggtcgagtgcttctgctttaccgataacacgttgaagccgggtgaagaccttgaactgccggtcgttttcttcgtggacccggaattcgtgaatgacccggacctgaaggatgtgaagacgatcacgctgtcctatactttcttcccgattgataagccgaggccggtggtgaacgcgaaggcagtgggcagcacccgcaacggcggc

SEQ ID NO: 38 - DNA, 1116 bp, Brucella suis
atggaaaaaagccgtaaatcgcatgacaggtccgtcctgcgttttttgcgtcatctactggaaaacgagtacgccataatgacccaacagcgcccccctctggagatccgcctttgcggcccgcgcggcttttgcgcaggtgtggaccgggccatccagattgtcgtcctggcgctcaaaaaatatggtgcgccggtttatgtgcgccatgagatcgtgcacaatcgttatgtggtcgaggggcttcaggcccgcggcgcgatcttcgtcgaggaactggatgaaatcccggcggcacaccgcaaccagccggtggtgttttccgcccatggtgtgccaaaatccgtgccagcggacgcggaggccaagaacctcttttatcttgatgcgacatgcccactggtttccaaggtgcacaaacaggccatgcgccaccagcgccttggccgccatgtcatcctgatcggccattccggccacccggaagtgatcggcacgatggggcaattgccggatggcgcggtcacgctgattgaaaccgtcgaggatgcgcacacctgccatttcgatgatgaagacaatcttggcttcgtcacgcagaccacgctgtcggtggatgacacggcgggtatcatcaaggaattgcaggcgcgctttcccaatctggcggctccggcggcggaatcgatctgctacgccaccaccaaccggcaggatgctgtgcgtgcggcggcgccgggctgcgatctgtttctgattgtcggtgcaccgaattcctccaattccaagcgtctggtcgaggtggcggaaaaggcgggcgcgcgcatgtccatgctggtgcagcgcgcggaggatatagaatgggagcagatcggcgatatttccgtcgttggcctttcggctggcgcgtctgcgcccgaaatcatcgtggatgaaatcatcgatgccttcaaagcccacttcgatgtgaagatcgaactggcggaaacaacggttgaaaccgaaaatttccttgtgaatcgcgaaatccgcgatgtggaattgacggtgaaggatatggcattcgtcaatggcgagcaccgcgtggtcggcatttccaaacttatgcaggggaag

SEQ ID NO: 39 - DNA, 1401 bp, Brucella suis
atgagccggttttccgcactcatgcgcaccaccgcggcgcggctttccgcgctttatctcctgctctttgcagtgggcgcggttgcacttgtcttttacatgaccaacctttcggcttccattctggccgggcagacccaacaggcgctgggcgaggaagtcgcgagcatcggcaaaagctatgcgcgcggcggtattccgcaacttgtgcgaaccattgattatcgctcgcgccagccgggtgcctatctctatcttgtcgccgaccctacgggccgtattcttgccggtaatgtagaaagcgttgaaccgggcgtgctcaacacggacggtattatcgaacgtgcctttacctatcgccgttacggcgaacaggcgccgcagatggagcatcgcgccattgccgtggtgattgccctgccgaatggaatgcggctgctcgtcgggcgggatctgggcgaacctgagcgatttcgcgatctgatacgcaactcgctggtgctggcgcttggcatcatgggcgtgggtgcgcttttgatctggctcttcgtggggcggcgcgcgctcaaacgcatcgacgatgtgtcgcgcgcatcgcaacgcatcatggatggcgatctgaccggcaggcttcccgtcaacggctcgggcgacgaattcgaccggctgtctggcaatctcaatgtcatgctggcacgcattctggaattgaacgaggggctgaaacaggtctccgacaatattgcgcatgacctgaaaacgccgctcacgcggttgcgaaaccgcgctgaggaggcgcttggcggtgagaaggtagagccggaatatcgcgctgcgctggaagatattatcggcgaatccgatcagcttattcgcacgttcaatgccattttgatgatctcgcggcttgaagccggttattcatctgaaaacctcgacgatatgccggttgccccgatcatgcgcgatgttgcggaaatgtatgagccggtggcagaggatgcgggtgttaccttgacgcttggtgcgcttgatgacgtagccctgcacatcaaccgcgaactggttggccagaccgtgtccaatctggtcgacaatgcgatcaaatatgcgggcggcgaaggacgcacggcgacggtcacgcttttgatggaaaaggatgcgcaatgggtccatatcgtcgtggctgataatggccccggcatccccgccgacaaacgtgaccatgcgaccgagcgtttcgtgcgccttgaagaaagccgcacacagccggggtctggcctgggtttaagtcttgccaaggcggtcatgaagttgcacggtggcgcgcttcgtcttgaggacaacggacccggccttcgcgccgttctggaatttccgctgccgcatcgcgaggttggc

SEQ ID NO: 40 - DNA, 798 bp, Brucella suis
atgaaaatctgtacctctattctcgctcctttcatactggccgtgctggcaatccctgcacaggcttcaacttccgcctggaccgaaactccgggcgggcgtgtgcgtattgttatcgaaggtggcgatccggcaatcaccggaaaacaggaggtgcgcggggccttgcagatcgaacttcgccccggctggaaaacctattggcgcaatcccggcgatgccggtgtgccgccgcagatcagcattgagggcgatgcgaaggcgcaaattgcgttccccgcgcctgtacgcttcgccggggacgaggagggcggcatcggctacaagaagccggtttcgctgcccgtcaccttccacatcgagccggggggcaagcggctgaaaggtcatgccttcctcggcatgtgtgaaaatatctgcgtgccggtgctggccgaattcgattttccgctggatgccgacaggcaaaaatcatcatcggagaatctcgccgcacgcaccgtcgtcgagaccgccttcgcacaactgccggcacctgcttcacccgatttcggtgtgagcgcggtcaagcacgacaaggatgaagccatcttcgacgtcgccctgcccgatcccaatgcgcctgccgagcttttcgtggcaagcgacacggtcaatctttccgcgccggtttccgtcgaggatggaaaagccagccagcgttttatcgccaagatgcagggcaagacgaaagatgccgttatcgactatacgatcgtgcagaatggcaaggcagtttccggccggctggaactggac

SEQ ID NO: 41 - DNA, 1014 bp, Brucella suis
atggctgaaatattttcagccggagattcgcaccgcttacttttaaggaatagcgtcgtgaacgcccatacgcgcgcagccgccgatgcggccaggcttgaaatgaatccgatgctgggcatcggcctcaaaattgcttccgttgctgttttcgtcgccatgtccaccttgctcaaggcggcggaaggcgttccggtcggccaattgatcttcttccgctccttcttcgcaattttcgccatcctgctttatctgggctggcgcgggcagcttggcggggttttctctacccgccacggcttcagccatttctggcgcgggctggtgggtgtgtgttccatgtcgatgagcttcttcgcgctaacaaagttgcctttgccggaggccatcgccatcaattatgcctcacccctgattaccgttattctgggggcggccatcctgcacgaagtggtgcgcttctaccgctggagtgcggtgctgatcgggctgttcggcgtcatgattatcatctggccacgcctgaccctgttttctgccgaaagtgtcgggcgcgacgaagcggtgggtgcgctggccgcgcttggggccgcggtcatgtcggctgtcgccatgatgctggtgcgccgtctggtccagaccgaacgtacgccgaccatcgtgatctatttttccatatcggcaagcgtgatcgcgctggtgagcctgccgttcggctgggttgtgccgaactggtcacagcttgccatgctggtgggggcgggctttgcgggcggtatcgggcaaattctgctcacggaatgctatcgccatgcacccatgtctacgattgcgccgtttgaatatacttcaatgctgctggggctggtgatcggcttcatgctcttcggcgatataccgacctttgaaatgttgatcggcagcggcatcgtgatggctgccggcggctttatcatctaccgcgagcaccagctttcgttggcgccgcacaaggttaatgccccgcaggcgcaa

SEQ ID NO: 42 - DNA, 819 bp, Brucella suis
gtgtcttcctgtcgcgattcggacaggaaacagacaatggctgcaaggaaacgaagctcctcttcgcgttcgccaaagcgctcttccaggcgcaaaagcggatcgggttcctcgacgccgatattggcgctgggcgccattctggctcttggcgcattcagcctgtggtccactgcgcagcacaaaagcccacaagcggctttatccgcgctattccagcgtcccgcgcccaagcccgccccggcagctacagccaaggctgaaaacaagcccgcgcccggcaagactgccgatacgaaagcgccagccagagattatgcggccgccgttggccctgttccccgcccggcagcaccggttgcgcccacaccgcaaaagcccgtccagaccgcggccgtcgcggcgcccaaaccgcccctcccttccgggagcgtcgtggcatcggttacacccgctgcaccttacgccatgccaccacgcggcgtcaatacaccggcaaactcgccgtcggttatctatgccaaggccaagctcaccattcacaagaatgcctgggaccgttcgccagccattgccaccattgagaaaggccgcgaaatgcgctcctacggcaagacaggccgctggcatcgtgtggttgtaccctcgacgaacatcatcggctgggttcacgaggatcaattgatcggtggacacaacaagccggacagcgcaacgctcattaccggatcagttgccaaaaaggcgccatcgcccgcgtccacgcccgctcatgtgcaatcacacatcctgccgccaaaggctgtcggcgctaaaaac

SEQ ID NO: 43 - DNA, 360 bp, Brucella suis
atgaagagaatccttctagctgaagacgataacgatatgcgccgtttcctcgtaaaagcgctggaaaaggccggttatcacgtgacacactttgacaatggtgccagcgcctatgaacggctccaggaggaaccattttcgcttttgctgacggatatcgtcatgccggaaatggatggcatcgaactggcgcgccgtgcgacggaaatcgatcccgatctcaagatcatgttcatcacgggctttgccgccgttgcgctgaaccccgattcggacgccccccgcgatgcgaaggtgctttccaagcctttccacctgcgcgatctggtgaacgaaatcgaaaaaatgctgatcgccgcc

SEQ ID NO: 44 - DNA, 918 bp, Brucella suis
atggctgatcaggctatatccgatacacgtctttcagacggttcgcgcggttttctgatggcgctcggagcttatggactatggggcgcgttgccattttatctcaaggcgctgtctcacgttccggcgatggaagtcgtctcgcatcgcgtcatctggtccgtgccggtcgcgcttgtgctgttgatcttgcttggtcagttcaggggcatcctgagtgcagttcgccagccgcgaatgctggccatggctgcattgacggcggcgctcgtcactatgaactggtctgtttatgtctgggcggttgcacatgaccaggtgatgggggccgcgctcggctattatatcaatcccttgttcaacgtggttctgggccgtttgtttctgggcgagcggctgacaaaaacacaatatgtcgccgtgggtctcgcctttgtggcggtcgtgcttctcactatcaatgcgggcggcttgccctgggtttcgctggccctgccgatcagcttcggcctttatggcttcttccgcaagtcgttgccgatgccgcccttgcagggttttacgctggaagtgctgatcctttgcatccccgcgcttgcctatgtggcatggctcgccgcaacggggcaggaccatttcgttgcggggcccgtttccaatattacgcttctgttgctggctggacccttcaccgccattccgctcattctttatgcgggcggggcgaagcttctgcgctatgcgacacttggcctgatgcagtatatgacgccgaccatgctgtttttcttcgccatattcatcttccatgaaccgttctcgacggcgcagcttgcagcattcttgctcatatgggcgggactggcgctctacacatggtcttcgctttcccagcacaggcgcaaccgcgcgcaggcc

SEQ ID NO: 45 - DNA, 1068 bp, Brucella suis
ttgcaggataaaacagctctcatcgtcggtgtaaccgggcaggatggcgcctatctaagcgagcttctgctgggcaagggctatcgggtgcatggcctgaaaaggcggtcatcgtcattcaatacggcgcgtatcgaccatctttatcaagacccgcatgaggaagacatccgctttcgcctgcatttcggcgacctgacagacgcaaccaatctgtgccgcgtcatccaggaagtgcagcctgacgagatttataatctcggcgcccagagccatgtgcaggtgagttttgaaacgccggaatatacggccaatgccgatgcgctcggcacactgcgcctgcttgaatccatgcgcatcctgggtctcggcaaaagctgccgtttctatcaggcttccacttcggagcttttcggcaatagttcccaccatgcgcaaaacgagcagacgcctttcgctccgcgcagcccatatgcaaccgccaagctttatgcttactggacaacggtgaactatcgcgatgcctatggtttccacgcctcgaacggcatcttgttcaatcatgaaagccccttgcggggcgaaaccttcgtcacccgcaagatcacgcgcgcagttgcagctatcgaacgcgggctacaggacaggctgcggcttggcaatcttgaggcccgccgcgactggggccatgcgcgcgattatgtcgaaggcatgtggcgcattctacaggaagacacaccggacgattatgtgctcgccacgggtgaaacgcatacggtgcgtgaattcatcgaacacgccttcaaggccgttgacaagcagatcgtatggcatggcgatggcgtggacgaggtcggcatcgaccgcaagacgggcaattgcctgatcgagatcgacccgcgctatttccgccccaacgaagtggatttccttatgggcgatgcttccaaggcaaaagaccgtctgggctggcaacatacgaccggctttgaaaatctcgtggctgaaatggtggattgggatctgcgcaacatcatgcgggaggtaggacgcaatgatttctacggc

SEQ ID NO: 46 - DNA, 678 bp, Brucella suis
ttgtgtcattcgcaatcgatccgtctatctgccgatgtcgccgcagctttcctgaaggtatttgccatgacgatcacgcaatccggttctccacactccattgccggcagaaaacaaacctcccgcccgcttgaaggcggcgcaacctttacgctgaagcatcgcctgcagcggcttttgtggcaggctgcgtggcttgcccttgcagcgtggaccccttcatgcctgtggcgatggcgcggcatggtgctaaacgccttcggagccgatatccacaagagcgcaatcgttcgcgcaagcgcacatatctggtggccgggcaatctgtccatgggcgcacattcctcgcttggccccggcgcgatctgctacaatgttgcgccggtaacgctcgcccccttcgccattgtctcgcagcgcgcgcatctttgcacggcaggccacgatatcgatcaggcgagctttcccctcactgccgcaccgattcacatcgctgcctatggctgggtggcggcagaggcttttgtcggccctggcgtacatattggcgagggtgcagtgctgggtgcgcgcagcgtcagcttccgcgatctggatgcatgggccgtttacacaggcaatccagcaaagcccgtacgccagcggcgcaaaaacacagattattcaatccaccggttt

SEQ ID NO: 47 - DNA, 963 bp, Brucella suis
atgtccacaatccacttcgataaaatcacgctgatcggtatcggccttatcggttcctcgctggcgcgcgtcattcgccgcgagggcctggcaacgcatatcgccatcgccacccgcagcgccgaaaccctgaagcgcgccgaggaattgaacctcggcgacagctacacgaccaacagcgccgaggccgtgaaagatgccgatctcgtcatcgtttcggttcccgtcggatcctcaggcacggttgcgcgccagatcgcgggcaatctcaagcccggcgccatcgtcaccgatgtcggctccaccaaggcttccgtcattgcacagatgcagccggaactgccggaaaatgtgcatttcattcccggccacccgctggccggtacggaatattcaggcccggatgcgggctttgccgagcttttcaccaatcgctggtgcattctcacgccgcttcccgacacggatgaaaaggcgctggaaaaactcaccgccttctggacagcctgcggctcgcgcctcgaccatatggacccgcagcaccatgatctggtgctggccatcgtctcgcatcttccgcacatcatcgcctacaatatcgtcgggacggccagcgatctggaactggtaacgaagtcggaagtcatcaaatattccgcatccggcttccgcgatttcacgcgccttgctgcatccgacccaaccatgtggcgtgacgtctgcctgcacaacaaggatgccattctggaaatgctgggccgtttctcggaagaccttgcctcactgcaacgctccatccgctggggcgatggcgaagccctgttcgatctcttcacgcgcacacgtgcggtgcgtcgcggcatcatcgatgccggtcaggaagtggatgcaccggatttcggccgtcaggcagccgtacgctccacggcccccctcaaagcgccggaaggcgacccagcggaa

SEQ ID NO: 48 - DNA, 570 bp, Brucella suis
gtggatgccataatgatggagcataggaacgatttctgggtcttcggctacggatcgctgatgtggcggccgggctttgcacatgttgaaacggtgcgtgcccgcctgcatggctatcgccgcagcctgtgcatctattcccacgttcaccgcggcacccccgaccatccgggtctggtgctcggccttgatacgggcggttcgtgcctgggcatcgcctttcgcgtgccgggcgacatgacagacgaggttatgacctatctgcgcgaacgcgaaatggcgaacaaagtttatcacgagaaaaagcttcatttgcggcttgccgatggacgcatcgttgaagccgtcgcctatgttgcggatcgccgccacaggcaatatgcgggccgcatgacgagtgagaaagcagccgccatcattgcttcggcacagggcgaatccggcgcaaatatcgactatgttaccaatacgctcatgcacctgcgcaatatgcgcgttcgcgatcatgcgctcgaacacataaatgagcttgttctggctgcggcaagccggcaggcaaacaatgcagcc

SEQ ID NO: 49 - DNA, 399 bp, Brucella suis
atgaaaaagctgcttattttggccgttgggatgatgctggctggctgcattactgatgctggaaaactggaagatcggattgccgctcaaagagcgccttcaccggccataaagcaactcatcatcaatgaggcgcggaatatcctgctcgatccatattcggtccgcgatgtctcgatttcatcccttattccgaatctgaaaagctccaataatgatgggttcatatgcgtccgcttcaatgcaaagaatagctatggcgcttatacgggtctaaaaacggtcggaacaaacgtcattagtgggcggctacttggatattttaccaatccagccatatgcgcatatcctaccgtgaagtggcaccccttgccggaagtttcgcaactgcgcaaactg

SEQ ID NO: 50 - DNA, 474 bp, Brucella suis
ttgggattgctggaaacggccattaccgggcgcaagcggcataacagccacgcggtcgtggtgggtgaactggggcgcggcatcgtcgcggggaccattcccgaagggtccatccttccgggcgatcatgaattgtcgctgcgcttcggcgtatcccgcaccgttctgcgcgaggcgatgaaaacgcttgccgccaagcgcctgatcgagccaaaagccaaggtgggcacccgcgttctgggccatgcaagctggaatttcttcgatcccgatgtcctgtcatggcgctggaaccggctgctgctgccgccgccgagcgcgccaccagcgacgagatcgtggagctttatgcacttgccgccaaattcgacaatcccgaccacacgccggaatccatcgccaaggtcgatctggaattccatcttgccattgcccgcatgtccggcaatcctttcatgcgttcggtcagcggct

SEQ ID NO: 51 - DNA, 144 bp, Brucella suis
gtgctgacagaagcgatttcatggggcgtcaactttcattctggaaataaacggacacggggccgtgcacaccggcgcggcacggccggtgcggtagagatcgggatatgccttggagggtttccccccgacccgcccggcaaa

SEQ ID NO: 52 - DNA, 801 bp, Brucella suis
atgcgtagtcgcagtttttcatgttccgctatagcgatggctctaggcttggtccttaccttggcacttgcctgccatagtacttttgctttcgatctgaacgacgattcagaaaaatcgcgaagccccttcgagcttttcaaattcggcttctctgcctacaagaacgggcataaggacgaagccatcaaggctttgcgttatgcggcggagcgtggccatcagggcgcgaagtggaagctggcgcgtatgtatgccgagggcgacggcgtggccgaggatgattatgaagcctataagatttttgagaagatcgttcgtgaaggctccgaacccggctccgaaaacgctccctacgtcgcagatgcgcttgtcgcgcttgcaggctatgtgaaaaacggcattcccggctcgccggttcaggccaaccccaacatggcgcgtgaactttatgtgcaggcagcggccaatttcggcgactctattgctcagtttgagcttggcaagatgctgctcgacggcgaaggtggtgagcgcaacgctgttcaggctgcacgctggttccagcttgcggcccggaagggcaataccggtgcgcaggccatgctcggcaatatgctgtttcaggccggcaagaccgtgcgtggccttgccatgctcacggctgccttcgagcgatgcccggccgaagactgcacctggatacgcgacatgcaggaacaggccttttccattgcaggcgaggccgaccgccgcaacgccatcgcgcttgcaagtgattacgctgtaaaaggcagttac

SEQ ID NO: 53 - DNA, 147 bp, Brucella suis
atgctggcgcgtgaacagatgcagcgcaagctgaagggccgccgctccaatycccgcctgccgcaactgatggaactgatcctgcgcactccgctgtttcaagtcaactggtcgaaaaggaattgcaggtcacacagcagggcgcgc

SEQ ID NO: 54 - DNA, 1278 bp, Brucella suis
atgactgtcgcagaagcggttgccccacaggcaaactccgtgcgtcgtgtcttgacggcaagcatgatcggcacgaccattgaatttttcgatttctatatttatgcaacggctgcggtgatcgtatttccgcacctgtttttcccggcaagcgacggcaattccgcactgttgcaatcctttgccactttcgccatcgcctttttcgcccgccctgtcggcgctgcgattttcggccatttcggtgacaagatcggccgcaaggccacgctcgtcgcggcactgatgaccatgggtctttccacagttgccatcggcttcctgccaacttatgcctccatcggcgttgcggctccgcttctgctggcgctctgccgtctgggtcaggggctgggtcttggcggcgaatggggcggtgcagtgctgctcgccaccgaaaacgcaccggaaggcaagcggacctggtatggcatgttcccgcagcttggcgcaccggcaggcttcattctggccacgggcattttcctgctgctcgccgaaaccatgaccgaagaacagttctttgcctatggctggcgtgttcccttcatcgccagtgcaattctggtggccgtgggccttttcatccgcctgcggattgccgaaacaccggagttccagaaagccatcgacaaggctgaaagggtcgccgtgccagccgcacagcttttcaggcatcacaagatgaacctgttccttggcaccatcggcaccatggcaaccttcgtgctcttctatctcatgaccgtgttttcgctcggctggggcacgcgcgcactgggctacagccgcgaggaattcctcgtgcttcagatgatcggcgtcatcttcttcggcctcaccattccgctttccgccctgctctccgaccgctacggcatgcgcaccatcatggtcatcgtcaccgtgctgatcggcctctacggcttcatcatggcgccgctctttgcggcgggcaccgcaggcgtgttgggctttctcatcatcggctttgggctgatgggcatgacctatggccccatcggcgcggttcttgccgaacccttcccgacttcggtgcgctatacgggcgcgtcgctggccttcaatctggccggtattctgggcgcgtcgcttgccccctatatcgccacctggctcgccaccgattacggctttgcctatgtcggctattacatggtggcagcggcaatcatttcgctgatcggctttgtcttcataacgctgaaagacaagaaa

SEQ ID NO: 55 - DNA, 522 bp, Brucella suis
gtgatgaagtccttatttattgcatcgacaatggtgcttatggcttttccggctttcgcagaaagcacgacggtaaaaatgtatgaggcgctgccgaccggaccgggtaaagaagttggcaccgtggtcatttccgaagccccgggcgggctgcacttcaaggtgaatatggaaaagctgacgccgggctatcatggctttcatgttcacgaaaatccaagctgcgctccgggagaaaaagacggcaagatcgtaccggctcttgctgccggcgggcattatgatccgggtaatacccatcaccatttagggcctgaaggtgatggacatatgggcgatttgccacgcctgagcgccaatgctgacggcaaggtgagtgaaaccgttgtcgctccacatctcaagaaattggcggaaatcaagcagcgttctttgatggtccatgtcggaggggataattattccgataagcctgagccgcttggtggcggtggtgcccgttttgcctgcggcgtgatcgaa

SEQ ID NO: 56 - DNA, 1326 bp, Brucella suis
atgggaccgatcacggataaggtggcgacagccgaaagtctgccacaggaaaccaggattgccattatcggcggcggcgtgatcggtgtttcgacagcccttttccttgccgagcgcggcattcccgtcaccctgtttgaaaagggtgaaattgccggggaacaatcgagccgcaactggggctggtgccgccgcaccgggcgcgacagccgcgaaatgcctttgatcgtggaaagcatgcgcctttgggaaggcatgaatgcacgcacaggtcgcgaaacgggattcagagtaaaaggaattgtctatgccgctgaaaaagaagaacaaatggctcgttatgccgaatggatcaagattgcccgccagcacggcatcgagacccaccttctgaacggcgaacaggctgaggcgcttgcgccgggcctgacacgccgccttgcgggcggcatgtcaacaccgctcgacggacgtgcagaaccccagaaggcggttcctgcaatggcagcaaaggcgcaggaagacggtgctgtcatcctgcagaattgcgcggtgcgcggcatcgaaaccaccaatggccgtgtgagcgctgtgctaaccgaaaagggccgtgtgcgctgcgaggcggtcgtggttgcaggcggcgcatggtctcgcctgattatttcaggcttcaaggcccatctgccgcaactcaaggtgcgctcctccgttttccgcaccgcgcctatcgaaggcggcgtggaacctgccattgccttttccgatttcgcgctgcgcaagcggctcgacggcggctacacggtggcgagcctcagcagctccatcgccgatatcgtgccggacagtttccgcttcttccgcgacttcattccctcgctttcgacggaatggcgttcgctgcgcttccgcctcggcgaacgcttcttcgaggaagccttccagtggaagctacgcccggcagaccagatgtccatcttcgagaaaatccgcactctggaccccgcaccgcaccatacggccaatgtgcaggtgctcgctcagctaaaaaaggcaatcccggcttttgcgccagccgtcattgctcaggaatgggccgggctgattgacaccatgccggatatcattccggtcatatcaccacttccgggtgtcgagggcctcatcgtggcgaccggcttttcaggccacggcttcggcataggccccggcgcggggcgtctcgtcgcagatatggtctgcaacgaaacgccgctcgtcgatccctctccgtttcatatctcccgcttttccgacggaacaaaaatccgcattgaaaactgggaa

SEQ ID NO: 57 - DNA, 135 bp, Brucella suis
gtggcaattgcgcaaaactggctctatatgcggctgatagataattcaaacttcggcttcagcatatggacgcacgcaatctgcaagatgaaaggcccggcgaggagaagtgccggcagcattccggcacggcat

SEQ ID NO: 58 - DNA, 1887 bp, Brucella suis
ttgcgacgtgtgggacaaggcgcgcgcttactgaacactttccctcgggggaaggatggaggaaatatgaaaaccgttcgactgaccgccgcgcaggcactggtgcgctatctggccaaccagatgacgccggagggcgacacattcatcgcgggcgtctgggccattttcgggcacggcaatgtggcggggctgggcgaggcgctctacggcgctcgcgagcatatgcccacctggcgcggccataacgagcagaccatggcccatgcggccatcgcctataccaagcagcttggccgcaggcgcgcctgcgccgtcacctcctccatcggccccggcgcaaccaatatggtgacagcggcagcacttgcccatgtgaaccgcctgcccgttctgttcgttccgggcgatgtcttcgccaatcgcggtcccgatccagttctccagcagatcgaggatttcaacgacggcaccatgaccgtcaacgactgtttccgcccggtaagccgttatttcgaccgtatcacccggccggaacagcttctcaccgccctgcctcgcgcctttcgcaccatgaccgaccccgccgattgcggcccggtgacgctcgccttctgtcaggatgtgcaggcgcaagcctatgactggccggaagaattcttcacgcccaaggtctggcactggcgccgcccgccgccggacgagcgggaactggaaaccgctgccgccgccatcaaaaccgcgctgaagcccgtcattgtggcgggcggcggcgtgcattattccggcgcgcatgaggtgctgcgcgcttttgtagaaacacatggcatccccgttattgaaacacaggcgggcaaatccgcactgccatgggaccatccgctgaatttcggtcctgtcggcgtgaccggcgctgacagcgccaatgcggttgcggcagaagccgatctggtgatcggtgtcggcacacgctttcaggacttcaccaccggctcatgggccctgttcaggcatccggggcgcaggcttctttcgctcaatgtacagccctatgacggagccaagcataattctctgccgctggtagcagatgcgaagattgggcttgaggcactctccggggccattggcgcttacagacgccaggccgtcgatgccagcctcaaaaacagatggtttgccgctgccgaccgtttcaccgccgcgcccagcgacggcaatgcgctaccgaccgacatgcaggtcatcggtgcggtgcagcggcaagcgcgcgaaaattccgttgtcatgtgcgcggcaggcaccatgccgggtgagttgcatcagctctggaaggccgggcgccccatgtcctaccacatggaatatggcttttcctgcatgggctatgagattgccggcggccttggcatcaagatggccgaaccggaccgcgacgtcatcgtgatggtgggcgacggttcctatatgatggccaattcggaactggccatcagcatcatgatggggcagaaaatcaccgtcgtcgtgaccgacaatcgcggtttcggctgcatcaaccggctgcaaatggcgaccggcggcgcggaattcaacaatctgctcgaccatgccgcccatgtgaacccgtcgcagatcgattttgccgcgcacgccgccgccatgggtgcggatgcgaaaaaggtcggctccatccgcgaactggagggcgcactggccgaagcacgcgacagcctgcgcacgaccgtcattgttatcgacaccgacccttaccccacgccggaaaccggcggctggtggtgggatgtcgccgtaccggaagtctcggaacgcgatgaggtgcgcgccgcacgcaaaaactatgaagcccagatcaaagaaagaaac

SEQ ID NO: 59 - DNA, 798 bp, Brucella suis
atgactgcccattcccgacaggacctcgaaacacggctggcgctggccgaaaagatcgtgcaagaggcgggcgccaaggcgctcgactatttcaatcgccgtgaaacgctggtcatcgaaaccaagcgtgacccgcaggacgtggtatcgatcgccgaccgggacgtggagcagcttatccgcgcccgtgtgtcggaaagctttccgcaagacgggtttctgggcgaggaatatgggctgaatgccggttcgtccggctatacctgggtggtcgatcccatcgatggcaccagtccgttcgtcaacggtatgcccaactggtgtgtttccatagctgtgttgaaagatggcgagccggtggtgggcgtcattttggcgccatgcttcaacgagctttatgtctccgccaaggggcagggcgcgacgcttaacggcaggaaacttgcgctcgacccgtcgcgcaatatccgcaacgcggtgacgggcatcggcgcgaacaattacgtcacgccgcaactggtgaccaaggtcgtcgagaacctgctggaagcgggcggtacttttatccgcaacggttccggcgcgctgatgattgcctatgtcgcggcgggcaggctggtcggctattacgaaccctatatgcacgcctgggattgcatggcgggcttctgccttgtgcgtgaggctggcggctatatctatccgttcccgacagatggcgaaaacctcacgaagggcaaccgcgttttcgccgctgcaccgggcgcggtagacgacctgaagaaagtcgctggccta

SEQ ID NO: 60 - DNA, 2073 bp, Brucella suis
ttgcaaattttcgtcttcggacgttcgtggatcggggcgattgcccttctgatgctcatatgcgctgttgcgcgctttttcctgcctgtcggaaagcgcagtggcgttctcatcgccgcaagcctggtcgggttcatttttctggctttgcaaggattggcaattggtttttccggctggaactggaccatcagcgaaacgcttttcgggccgctttccgatggccagccatctttcggcgcgggcgcgattctgacaggcctctgctttctcctgatcttctctttcgggcttgccgagcgcggtgtgatgaaaggcgacgcctttgttgtttcatcgatcacgcttctggtggcgctcgtcggcgtctttgtcttctatcccgtgctcagcatgttcgttggctcggtgcaggatttcgacggttcgttcaatccggatggctttatcggcaatattcaggattcctccatctggagccttgcctgcgtggtgggcgaggggcgctgcggtgtggcatggcgcacgctttggctggcgctgatgacggcgaccggttccacgattctgggccttgcctttgctctggtcgcgacccgcaccaactttcctttcaagaaagggctgcggcttctcaccattcttccgatcatcacgccgcccttcgtggtgggccttgcgctgacgctgctcttcgggcgtgcgggcgtggtaacggaacagatttccagcctctttggcgtcgagccgggccgctggctctatggcctcaccggcatctggatcgcgcaggttctttccttcacacccatctctttcctcgtgctgatcggggttgtcgagggcgtgtctccctcgatggaggaagcctcgcagacgcttcgcgccgaccgctggcgcaccttcaagcgcgtttcgctaccgctgatggcgccgggtctcgccaatgcctttctgattgccttcatcgaaagcatggccgatttcggcaacccaatggtgctgggcggaagccatggcgttctttcgaccgagattttcttcgccgtcgtcggcgcgcagaawgacccctcgcgcgcggctgttctcgccattgtgctcctttgcttcaccctgtcggccttcctcgtgcaacggctgtggctttcgggcaagaacttcgccaccgtcaccggcaagggcgattccggcatacatgctggcttgccgcgcgggttgaagatcggcgtctatgcgcttgtcattccttggatgttcttcaccgtggtcgtttatgtgatgatcctcgttggcggttttgtgcgccagtggggcctcgataattcgctgacgctggaacattatgcgcgcgccttctccgtctcatggaccgatggcgggctggcctggaccggcgttgcgtggaactcgttctggacgacgatggaaatctcgctcatgtcagcgccgctcactgccgcggtcggcctgctcacggcctatctgatcgtgcggcagcgtttcgtcgggcgcaatgtgtttgaatttgcactgatgctgagctttgctattccgggcacggtcatcggcgtcagctacatcatagcgttcaacctgccgccgctggaaatgactggcacggcgctgattctcatcgcctgtttcgtgttccgcaatatgcctgtgggcgtgcgcggcggcatcgcggcgatgagccagctcgaccaaagcctcgatgaagcctcgctcacgctgcgtgcgggcagtttccgcaccatccgcaaggtgatcctgcccttgctgcgtcctgccatcacagcggcgctggtctattccttcgtgcgcgccatcacatccatcagcgccgtgatcttccttgtttcggcccaatataatatggcgactgcctatattgtcgggctggtggagaacggtgaattcggcgtggcgattgcctattcctccatgctgatcgtggtgatggttgtcgtcataaccagcttccagcttctcgtgggcgaacgcaggctgcggcgcgaaaaccgcgttgcggcggctgcgcccgtcaaatccgtccgtcaggagaaaaccgca

SEQ ID NO: 61 - DNA, 195 bp, Brucella suis
  modified_base (190)..(194): a, c, t, g, unknown or other
atgaccacattgaccgcgaaatggaactttccgaccactgtgcttttcgggccggggcacatcaaggagctttcagccgtcctgaaagccgcaggcatcgagcacccgcttttcgtcaccgatccgggcctcgccaaactgcctgtggtcgcctcgacattgggccgtctggtatgcgcgggcgtgcatnnnnnt

SEQ ID NO: 62 - DNA, 834 bp, Brucella suis
ttgaactggctgacggattacaagatacctgtcgggccaaccgccaaatcggtggtggactggctgacgacccatctgggacttttcttcgacactctggccgcagtcatgcagagcttgattgacgatttgctgtatctcctggaactccctcaccccctccttctgatcgccatctttgcaatcattgcatggctcattcagcgtcgtatctcggtcgttctgctcaccattctgggtttccttttcatcatcaaccagggctattgggaacccaccctgaaaacactcaccctcgttctgtcatcatgttttgtctgcatggtgatcggcgtaccacttggaatcgccgcagcccatcgcccggccctttacgccgcgatgcgccctgtgctcgacctgatgcaaacgctgccgactttcgtgtatctgatcccggcgattgttttctttggtatcggcatggtgccgggtcttctcgccaccgtcatcttcgtgatccctgctcctatccgcctgacccagctcggcatttcctcaacacccgcctcgctcatcgaagccggcgaagctttcggcgcgtcacgctcgcagcttctctggaaggttgaactgccctacgcgatgccgcaaattctggccggcctcacccagacgatcatgctgtcgctttcgatggtggtcattgcagcccttgtcggtgccgatggcctcggcgtgcctgtggttcgcggccttaactccgtcaatatttcgctgggttttgaagcaggtttcatcatcgtcgtggtcgccatcgtgctcgaccgtatcttccgcgccggacgtgagaaa

SEQ ID NO: 63 - DNA, 1122 bp, Brucella suis
ttgcgacgtcaggccggtgctcgttccacgatcccacgtaacagtcgcagccgcagaccactggtcattgaacttatgaccgacaccgccgctaaccgtccaaccatcctgatagtaaaggttcaaactggtgatcttcgtgcctttgggagcgatcttgttatcagaagaaacaaagtccaccgatgtgatgctcgaccagtcggtccacttgaccgagccgaatgcgagccagtccggggcgataccggtctggaacttgaattcaaccgactgcggcgtagcaacatcgctctccacattaagcggcatgccccgccccgttatcgggttcagcacaagattgtcgatcgtgccttcaaggtcatacttaacttccgattgatagacgagcgtggcgcgcatcgcatattcaggaatttcgtaagcagcaccaagacgccagccaaccaactgatcttcagcatcaagcgatgctacccgaaccggccctggaaaaggaaacttcgtcggattcatgtttggactaatgccaggaatcagacgggtctgctcacccttcagttcctgatagctgacgccgccaagaattcggaaataacctttttcccctgctacaaagcgataggaacagttcacaccataatcatgagacgaaatcttctgctcgattgcggtgaacatgcgaactgtgtcggtaccgacatcagtctggataccccagggctgacgatattgcgccgcacaggcaagatcgctggtgatgtcgaatttggcggaaacctttggaacctagtagctcttggcctcatcgacttctgtttcatacggacgcccggtcgtgggattaatgccagccggaacaaaattctgccccccggtccactgaggggttttttccatagcagagccgcgaatattcttcagcttgcgttgtggcgcaacgaaggttccgccagcttcgacggcgttaccattttcaaaaagaatatcgaaatcctgcgagccacgctcaaacccacccgcatgagccataaccgtactgcccagcaaagcagctacgaacccagctattttcacgcgtcccatcttcatgaatgtcctcccagacagcaaaggcat

SEQ ID NO: 64 - DNA, 1557 bp, Brucella suis
atgaataagctcggcgtgtttatcggctataacccaggccaattagatccgtatcagggtatttctcgcttaattgcattcgtgatcaagggggccttgaaccagggtagcggtgtaacaattgcttgccccggctggctaaaggacgatgtacgtgttcttttggaagatgctgatatcccacttgaagcggtcaaaattatcgcgacgaatggtcagcctccattggcttcgttatggaagttgagagataagttccgtaagagacggacgagtaaacgaaaacgtctctggctggagcgctatggcaaaaatgttgcaaattttgttgcagaatggctttcttcgcgctcgtattgggggatttttttgggggctgctgcaattgctgtagtgactattctacttgccgtaccaattgctatagccttcaccgctcttatcggccttctatttgctcgtcggcttattagacgtgttatcaggtcaaagcttggtttgttttttcacaaaaatgccaatcaattcaacaaattaatgtcatctgatgaaaccatcgaccagatgagggaacgggaattctcattgttgatgaagaagatcaacgcccagaaggacatcaaagtttggtatgtaccagccatgttttggcccgaggtcgctaacataaaatccaagatcgtgatggcggccccggatatcgtcttcttcgattacccagggaacttccgagggatacgagagcataactcatacgatcgcatgttaaagagcttgaggtctgccgatcatctcgtttgctacagtgaaaatgcgaagcaaaaacattttgttgaacggtgtgatgttcctgcggaaaagataacggttatccgtcacgggttcgtagatttgggagcttctggtgcggctattttgcgtcaagatgcacttgacacattacatgcctttatcaagaagaatgatggtcgaatgccggaatatttgaaaggtttccggtttgacgatgttcctttcttcttctattcttcacagcttagaccacacaaaaacatagaaggccttattagggcttatgcaaaagtattgaaagagcatcagcgcccagcaaaacttatccttacggcacaatttcaatatgacaagcgcatccagacctttatagatgataatggcctgcatgctgatgtgctgtcgctgcattctgtaccaaacaaggtacttgctgctctatatcacctggcatcattgtcggtcacgccaacaaacttcgagggagggttcccatttacattttcagaagcctattcggtcggcactccatcgatcatgagccgtatcccagtcgtgcaggaagtgattgatgatccagagcttcaagacttgatgacattcaatcctttggatgtggatgatatagccaataagatgatttttggtttggacaacaggcaacgtctatttgaggcgcagtctagtctatacgccaaattatccgctcgcacatggcaggtcgccgcaaatgattatctgtctctgcttaaatctgttgcagac

SEQ ID NO: 65 - DNA, 1104 bp, Brucella suis
atgttgatcgtcggtgccggtatatctggtttggctttggcgcgtgcgctgcggcaattcgatattgaaacgcgcattctcgacaagcgcaatcaacatgcagaagccgggttggccatcaatctaccgggcaatgccattcaggcactggaacgtcttggcctgaaagaacaggttgatgcgcttggatatccgacgaagcggcgcgaatatcggacagcaaaggggaaattgctgttttccgtcaacgagaccgatttctggggcgagcgccatcagccgcgctgcatcctgcgctcggacttgctgcgcatcctgcaagaaggcaattcgcaagacaatcttctctatggcaagaaagtcgttgaaatctcgcaagatgccgggcgcgtttcggtggcgctttccgacaatgaaacaatggagacggcgtgcctgatcggcgcggatggcgtcaattccatcacccgcactctcgcattcaatggcaatgccgtcagggcggcagctttatccaattttagctggcgcttcatggcgccaaatccgggcgtcgattgctggtcgctctgggtgggctcaaaaagcatgatcctgcttattycggtgagcgagcaaagcgtttatgtttgggcgacactwmccggaaaccggaccgatagcggccaggtgcgccttagcggcgagcatttttccgatttcccctccgatctgaggtcgatcatcgaaaatgcggtcaagacgccccaggacgtctataattctcccttgcaggaagtccgcatggatcgctggtcagccggaaacgtattgctgctcggcgatgccgcccatgccacggcacccgtctgggcgcaaggcacagcacttggcatggaagccgccctcacccttgcaaaactcgtttcgcaaaaggtggaacagccggagcttttcgaccaatttgaaaagctgcatcgtccgcgcgtcaaccatgtcgtcgccatgacggacaagatgtccaaggcagccaagattccgtcctttgcgcaaaaactgctcctgcccattgtcgggccgcgcaattacgaggcaacatacggccccttgcgggacggcaatttc

SEQ ID NO: 66 - DNA, 1104 bp, Brucella suis
atgcagcgccttggcaacggcagcggaatcgtcagtcgaaccggcgcgttcgatgccggccttgatgacttccacagccgcataggcgttcatcgtgaaagcttcagccgggatgttcttcgccttcagggcttggatagcgtccttggcagccgggttcttcgtggcatccttggcgttggtgaagagcgtaccctgtgcgtttgtgccgccgattgcccaatattcggtgttggagaggccttcgccgccgagcaccagggcctgcatgccagcatcgtggagctggcgcgacaggagaccgccttcagcatggtagccgccgaaatagaccacttcagcaccggcagacttcagcttggtgacgagcgcggaaaaatccttgtcgcccggtgtgaccgaatcataatggacttcagtgatgccgcccttgttgatggccgccttgaacgcatcggcaagacccttgccataagcgcccttgtcgtggatcacggcaaccttcttgtccttcatgtttttcaggacgtaatcagccatgacttcagcctgctggtcgtcgcggccgcaggtacggaacacgttttccaggccgcgcgcggtcaggtccgggccggtagccgtcggggtgaccatgaggacgccgttttcagacaaaacgtcggaaacggggacggcaacgccggtcgtaaccgggccgacgacaaacttgatgccatcgccaacgatctggttggcagcggaaacgccctgcttcggctcgcccgcatcgtcggcaaacttcaggacgactttctcacccttgatgccgccagccttgttgatgacttccacggcggtttccgcgccctttttcacctgatcgccgaaagcggcaaccgggcccgtcagcggtgcaatcacaccgatggtgatgtctgcgtaagcggcgctggcaaagccgatggtagcggcaaaagccaagctggacagaagtttcaggttcattctttttctcctttggtggggcggcattgtccgcacagccccgaaacaacccatgccggtcaattttcagcgcctgcgcgctgccgaattgccgggccctcccttttgttcatcgcg

SEQ ID NO: 67 - DNA, 111 bp, Brucella suis
gtgtgttgtatgaattttataaaatttgacatgctttatttttatggagccgggaaatacaaaatgccggggcgttttcccgccccggcatttcatatcacgtcaagtcag

SEQ ID NO: 68 - DNA, 1380 bp, Brucella suis
atgtccgccgaaatcatttcccaaaaatccgccgccagccgccccatctggtttgtcgccaagaacgcgctggatgaagccgcccttcccgaaagcgccgctgcctgggcgaaggtcaatggcttcgatggtgaagccgggcgtgtgctggtgctgccggggcgcgatggcgcgattgcgggcgccctgtttggcaccggcgaggcgccggagaacggccagccgcagcttatcgccggaaagcttgcccgcaacctgccggaaggcgattggcatattgaaggcaagccgggcgatgccggtcttgccgcactcgctttcctgatgggcggttatagtttcacacgctatcgcaaggccaatggcaagacggtccgcctggccctgcccgaaggcgcggacgcttctgaaacacgccgtctggcagaagccgtcacgctcgtgcgcgatctcatcaacacacccgccaacgacatggggccggcggcgctggagcaggccgcacgcgatctggcggcacagcatggcgcgcgggtggcaaccacggaaggcgaagcgcttctggaaaagaatttccccatgatccatgcggttggccgtgcaggtgccgtggccccgcgcctgatcgaccttcaatgggggcgggacagcgatccgaagatcacgcttgtcggcaagggcgtctgtttcgatacgggcggtctcgacatcaaatccgcaagcgggatgctgctgatgaagaaggacatgggcggcgcggccaatgtgctgggtcttgccgcactcatcatggatgcgaaactgccggtacggctgcgcgtgctcattcccgcggtagaaaacgcaatcgcgggcaatgctttccgccccggcgacattctgcaaagccgcaaggggctgaccgttgaaatcggcaataccgatgccgagggccgtctggttctggccgatgcgctggcgcttgccgacgaagaagagccggaaatgctgatcgacatggcaacgctgacgggcgcggcccgcgtggcgcttgggccggatcttccgcccttctatacgcatgacgaccagcttgcgggcgaaatcgccgtaaaggccagagagaccgccgaccctctatggcggatgccgctctggcagccctatgcgcaaaagctttcctcgcgcgtggccgatatcaacaatgtcaccacggacggtttcgccggttccgtgacagcggcactgtttctcgccaaattcgtcgagagggcacgccaatgggcgcatttcgacattttcggctgggtgccggtcgaaaagcctgcctcccccgttggcggcgaagcgcaggccatccgcgccctttaccagcttttgaaggaacgttatccggcccgc

SEQ ID NO: 69 - DNA, 600 bp, Brucella suis
atgaaatcacggcgcatgttccgtcacagaggttcagttgcaattttgctcaccgcatttctggcggggtgcggtacaacaacgccgctcgacacgttcaacctgtccacgccccagcccatggtgaccgtaccgaagcgcaagaacttgcaattgctgatccccggaccagccgcgctgaaagctctggatagcgagaatatcgtggtcagctctgcgccgggctccatcgaatatctcaagggtgcgcaatggggcgacaggctgaccaatatcgtgcagtcgcgccttgtgcaggcttatgaaaataccagcgcatttggcggcattggccgtccgggcgatgggctggcgataaattatcaggttctgactgatctgcgcatgttcggcattcaggcctatacatcgccacaaactgctgttgtggaaatggccgtcaggctcttgagcgacaagaccggcgaagtccgcgccacccgcgtattccgcacggcaattcctgttacgggaaccagcaatgcggcctatgtgaaggctttggacaaggcttttgaccagactgtgaatgagatcgtaagctggacgatttcaaccctt

SEQ ID NO: 70 - DNA, 870 bp, Brucella suis
atgagtatcaacgcgaccacgctcgaccgccgcctcaatgcctatcgccccgatcttgccgatgagcggctgcgtggtcaggtggaggcatcccgcttcgttgcgggaacgccgatgcaggtttcggctccggtcgtcgatctgcgcgcagaaccgcgcatggatagcggcccgcagacgcagatgatctttggcgacaaggttcgcgttttcgaggatatggacggctggtgctgggtgcaggccgaacgcgacggttatgtcggctatgtttcggcggcttcactcgaaaggccggacggtgatgcaacacatatggtcgtcgtgccgcgcacctttgtctatcccggttccgatctgcactttccgcacacaatggcgctatcgctcggctcccgcgtgaagattgtcggcgaggccgaaacacgcggcacgaaatatgtgcttctggcaagcggcgaggccctgatcgccagccatgtcgcaccgcttggccaatatgcttccgattatgtagcggttgcggaaaccctgctttacacgccctatctgtggggcggcagttccggtttcggcatcgattgttccggcctggtgcagctttgcatgtttgtggcaggccggaatgtcctgcgcgattccgacatgcagcagaagacccttggtgaagtcgtgcagcctgatggtgcctacgccaatctgcggcgcggcgaccttgtcttctggaaggggcatgtcgcgatctgcacctcgcccgacatgctgatccatgcgaacggccacaccatgaccgtgacgctggaaccgttgcaggacgccatcaagcgcattgcctatctctacgacctgccaacattgatccgcaggccg

SEQ ID NO: 71 - DNA, 1098 bp, Brucella suis
gtggccgaaaccaatacctcgccttctcccgctcaagaccagtctcccaaacatccaatgctacgtcggattctgcttatcgtagggccggtcgtcgcgattgccatcggcctgtggatctatttcagtggagggcaatatgtgagcgaggatgattcctatgtcggcgcggcggcagtggccataacaccgcaggtttccggtcaggttatacgtgtcgccgttggcccgaaccagcccgtcaaggaaggcgatcttctgtttgaaatagacccacagccgttccagatcgaacttgaccaggcaaaggccaatctggcgcaagcgagcgaaaaactcgcagggcttgtcctcacctacaagcaacaattggcaagtgtggctcaggcaaaggacgatgtcaccttcgcacaggagcaattcaaccgtgtttcggcactggttcacgacaagtttgaggcgcgcgcgcaattcgatcaggctgagcgggccttgcaggtggccaagcaggagcaaaatgccgccgcgtcggcagccgccgcaacactggcacagcttggcggcacactcgataaaccgttacaacaacatgcatcctatctggcggccaaagccgaagtggagcttgccgaacgcaatgtcaggctaacccgcgtgctcgccccattcgccggaacagtgacgcaagtggaaaacatccagctgggaagttttcttgccgtaggcgatccggctttcaccctgatcggcgccgatgcctgggtaaacagcaatatcaaggaaacggacctgacccatatcaaggttggcgatcctgccacggtcgtgctcgattcctatccggaccagcccctgcaaggcgaggtgcagagcataacgcccgcaagcggttctgtttttgccctgctgcccgcccagaatgcctccggcaactgggtcaaggtggtgcagcgcatgccggtgcggatcaagattacaaaaccccatcctgacgtggtgatgcgtgatggcggcagcgcaaccgttaccatcaatacaggctatcatcgcacatttgctaccttgtggcgcgatctgaccggcatggtggggatcgac

SEQ ID NO: 72 - DNA, 108 bp, Brucella suis
ttgccggaccgattatgccggacaaatcattccaaattcatgaattttcgcgcgccaagcggcctcagcggcccctattatgcgtccgctcatatagctggcgaacga

SEQ ID NO: 73 - DNA, 396 bp, Brucella suis
  modified_base (387)..(391): a, c, t, g, unknown or other
atgcgcccgcgacggcgtgataacgaagggaagacacgtttgaaagagctaattgccgactccgatgccgcaggcaagcgtctggaccagtggctggccgctgcattggggccggaactctcccgcagccgcgttcagtccctgattgccgatggccatgtgaccatcgacggcgcgcccgcccgcgagacgaaacagaaactgcgcgaaggcatgaacatcgccatcgtgctgcctgagcctgaacccgccgaaccgcaaggcgagaacatcccgctcgatattctttacgaggacgatgatctcatcgtgatcaacaagcctgccgggcttgtcgttcacccaggcaatggcaactggactggaacgctggtcaatgccctcatnnnnnttaat

SEQ ID NO: 74 - DNA, 1539 bp, Brucella suis
atgtccagagccaggatttccaactatcgcaaaggcgttgccgccgtcgcgctttccgcagcgcttgccggcgcattcgtcgtaaccggcccgcttggtgcgctgaacgaagcccgcgccgaagcggtgcatgtgacgccgccgccgcaggctggctttgccgatctggtggaaaaggttcgccctgcggtcgtcagcgtccgcgtcaagaaggacgtgcaggagacttccaaccgtggtccgcagttcttcggccctcccggcttcgaccagttgccggacggtcatccgcttaagcgatttttccgtgattttggtatggagccgcgtggcgattcacgctccgataaccgtcgcggcaaggccaacaagccgcgcccgggccatgaacgccctgttgcacaaggatcgggcttcgtcatttccgaagatggctatgtcgttaccaacaaccatgtcgtttccgatggcgatgcctataccgtcgtgctggacgacggcaccgaacttgatgccaagctgattggtgctgatccgcgcacggacctggccgtgttgaaaatcaacgctccgaagcgcaagttcgtctatgtcgcctttggcgacgataataaggtgcgcgtcggtgattgggttgtcgcagttggcaatccgttcggccttggtggcacggtgacttccggtatcgtttcggcccgtggccgagacatcggcgcaggcccctatgacgatttcatccagattgatgccgccgtgaacaagggcaattccggtggtccggctttcgacctttccggcgaagtcatcggcatcaacaccgccatcttctcgccatctggcggcagcgtgggcattgcttttgcaattccgtcctccaccgccaagcaggtcgtggaccagctcatcaagaagggttccgtcgagcgcggctggatcggcgtccagatacagcctgtgaccaaggatatcgccgcttcgctcggtcttgccgaggaaaagggtgcgatcgtggcctcgccgcaggatgatggcccggctgccaaggctggcatcaaggcgggcgatgtgattacggctgtcaatggcgaaacggttcaggacccgcgtgacctggcgcgcaaggttgccaatatcgctcctggtgaaaaggcggccctgaccgtctggcgcaagaacaaggccgaggaaatcaacgtcacgattgctgccatgcccaatgacaaggggaagagcggcagccagtccaacgacaatgacggcggacagggtgaaacgctcgactcctacggcttgaccgtcgttccgtctgaagacggtaagggcgtggtcgttaccgatgtggacccggacagcgatgcggctgaccgtggcatccgctccggcgatgtgatcgtgagcgtcaacaaccagacggtaaagaccgctggcgatatcaacaaggccatcacggcagccgaaaagtcaggccgtaaggcggtgctgctgcaattgcagagcaatgatcagagccgcttcgtggcgctgccgatcaatcaggaa

SEQ ID NO: 75 - DNA, 2322 bp, Brucella suis
atgcgttgcatagatgcaacgctattggttattatggttaacatgaacagggcgaatctgacgaccgacatgaatgggacgaggcaatcatcgcaagatgaggggcagggtgaggggcgcaggcttctcgccttaccgggtattattaccgtcgtttcggcgctggtcacagcttcgttttccttcgcgatcctgatcggtattacgccaatcacccccgaccgcacggttacactggcgctggtgattatcaatgtcgcgttgattttgtttctgatcctgctgatctgccgcgaggtctatcgcatcataacggcgcgcaggctgggcaaggccgcgtcgcgcctgcatgtgcgcattgtagccctgttttcgctgattgccgccattccggcaatcgtggtggcaattgtcgcttccgtgaccctcaatctgggccttgatcgctggttcgaccagaatacacgcgacatcatcagttcatcccagaatttctatacagcctatcttcaggaaaccgcgttgaacctccagagcacgtcctattcgatgttgcaggatctggatgcgcagcgtgccatttatagcctcgaccggaacggtttcatccagttgatgacattgcaggtgcgtgggcgcgccttgctgggcgcatttctggtgcgtgataatggcgatgtcgtcgtgcagagcgatacgggcatggaaagccatctgccgccgccgacgaaggattcgcttgaaaaggcaccggacggaaagcctgtcatcatcccgccgggaaacaccaatttcgtcggtgcactcatcaagatgcgcgaaattcccgacatgtatctttatacggtgcgttcggttccgccgcaaattcttgatgccatcaatctgatgaatgccaatgccaggcgctatcaggaaatggatgcgaaccgcattccgacgcagattgctttcgctttgctctatttcggcctgacgctgatcgtgcttctgtcggccatctggacgggcattgccgttgcagaccggctggtgcggccaatccggctgctgatcggcgcatcggacgatgtggctgccggcaatcttgatgtttcggtgccggtgcgctcgtccgatggcgatgtgggggcgctgtcgggtactttcaacaacatggttgccgagctgaaaagccagcgcaacgaacttatttccgccaaggaccagatcgatgagcgccgccgcttctccgaagccgtgctttcgggtgtgacggcaggggttatcggtattgagaatgacgggtcgatttcgatcctcaaccgctctgcggagcatatgtttggcgtaacgtcggaagatgcggtcgggaaaagccttaccagcatcgcgccggaggtcgggcaggctttcgaggtggcgcgcgccacggggcgaaccgtgcaccgcgagcaggtgagcatggcgcgcggcggcggtagccgcagcttcaatgtgcaggtaacggtcgaggatgccgaatccgaggatcactcctatgtggtgacggtggacgacatcaccgatctggtgcaggcgcaacgttcttccgcatgggcggatgtggcgcggcgcatcgcgcatgaaatcaagaatccgctgacgccgatccagctttcggcagagcgcatccgccgccggtatggcaaggtcatcacggaggatcgcgaggttttcgaccagtgcacggacaccatcattcgtcaggtcggcgatattggccgcatggtcgatgagttctccgcttttgcgcgtatgcccaagccggaaatgcggcatatggacctgcgcgaagcattgcgtgaagcctcgttcctgattgaagtcagccgcagcgacattcatttcgacaatgatttcggttcggaaaagctgattggatcgttcgatagccgcctgatcgggcaggccttcggcaatgtcatcaagaatgccagcgaggccattgacgcggttgcgaaggaagagcgcggcgaggggcatatccgcattcacgcctacagggcggatgggcaactggtggtcgatgtgatcgacaacggcaagggcttgcccggcgatgatcgccagaaactgctggaaccctatatgacgacccgcgaaaagggaacgggccttggcctcgcaatcgttcgcaagattgttgaagatcatggcggtaatcttgaattgcatgatgcgcccgcggattttcacgcaggccggggcgcaatgatacgtatggtctttcccgaggatccccccattccatccgatgcggattcgggaaatgacgaaaagcacacagctggacaggtgatt

SEQ ID NO: 76 - DNA, 564 bp, Brucella suis
atgacgatgtttcttgtgacgccaccggcgctcgagccggtgacgattgccgacgcacgcgcgttcttgcgaatttctaccgagagtgaggatgatatccttggccgcctcatcaccaccgcgcgcgagttggtggaagccgaaacggggctggcgctcattgaccagacctggcgtctgcgcgttgatcgctggccgcgttcgggccgtctggcgctgttcaaatatccgatcaaagcggtgacgggcgttgttgcctatcggccggatggggcggcgatcagctttttgccggacgaatttgcgctgcatcatgacaggcgcccgcagcgtctttacatggcgcaatatcccgatgcctccacattctgcggtatcgaagtggatttcattgcaggctttggtgagagcggcgttgaagtgcctgatacactgaaacaggcaatccttaccctgaccgcccatctttacgaatcgcgcgcaggtgtgaacgccgatgccgttcagcgatctttcccgcccatcgtgaaccagatggtcgatagctggcggcggatttcccta

SEQ ID NO: 77 - DNA, 807 bp, Brucella suis
gtggaggatggcagcatgacgaggcagatttcggcagcggaaacgcgcattatccgttttttgaagcagggcggttgcgagcttcaggattcggttcgcgaaacgcacgctttgctggttggcgaacaggggactattgcggcttcgcgccttgaaatcgaacggatgcgcaaggctggaaccatacaatgtgacgggcagcgcatcgcgctttctgcaatgggacgcaagcgtcctaaagagcagaaaaagcccacgctaaccgccgcctctgtccgtgagacatacgagatagagccgggcgaaaaggtggagatcaatccggcggaatccccacttgccatgctttatcggcgccggaacgccgatggccgcgcctttatttcggaggatgaattccgtgcgggcgagcgcctgcgcgctgatttcacgcgggggtcgctcatgccttccgttacgtcgcgctgggatgtccaggccgggtcgggcgggcattatggggcaggcggcatggctgaactgaccgatatcgcccttgcaagccggataagggtggagcgtgcgctggaagctgtcgggccggaactgagcggcattttgatagacgtttgctgctttttgaaaggactggaaacagtcgagcgtgagcgtcaatggccggttcgttctgccaaggtgctgctgaaagcggggcttgccatgctgcatcgccattataatcctccgcgcgaaggcgaaaggcgaaggggggcagtcctgcattggggggcggaggattatcgccctgagattcgaccgcggagtgtg

SEQ ID NO: 78 - DNA, 1137 bp, Brucella suis
ttgcaggaggctctggccctgtttttcggtagcgaaagtctgttggtcgggacaatcataccgtttctgttcgttttgaccgtcgtggtgttcgttcatgagatgggccactatctcgttgcgcggtggtgcggcatcggagcccaggctttttccatcggcttcgggccggaactgctgggcttcaccgaccgccatggaacgcggtggaaactttcggccatccccctcggcggttatgtgaagtttatcggcgatgagagcgaaaccagctcgcctgtaggcgtcaacgagagtgctctcagcgaagaggaccgcaagcgggcctttcatacccaacccgtctggaaacgcgcggcgacggtttttgcaggtccggcgttcaatatcattcttaccattgcaatcttcagtgttttctttgcgctttatgggcgccagatcgccgatccgctgattgcgggcgtacagccgggaagcccggcagccgaagccgggtttgaaccgggggaccgcttcgtcagcgtggagggcgagaagataacgactttcgccgatgtgcagcgcatcgtttcaggccgtgccggcgacaagctgaattttaccgtcgagcgtgacggcaagatggtggaccttcaggcagtgccgaagattgtcgagcggaccgatccgcttggcaacaaggtgaagcttggcgctatcggcgttgaaaccacggaagctgtgggcaatttccggcgtattgaatatggcccgctcgaatcggtggggcaggccgtcattgaaaccggccatattatcggtcggaccggcgagttcttcaaacgtttcgccgtggggcgcgaagacaagtgccagcttggcggacctgtgaagattgccactatggccagcaaggcggcaagccaggggtttgactggctgatacaattgatggccatgctatccatcggtatcgggctgttgaacctgtttccgctcccgccgctcgatgggggacatctggttttctatgcggtggaggcaatcaagggcagccctgtttcaggtgcggcgcaggaaattttttatcgcatcggttttctccttgtcatgggatttatggggttcgtacttttcaacgacctttttgcctgc

SEQ ID NO: 79 - DNA, 810 bp, Brucella suis
atgtcaaatctgcaaacccgcatcatcaccgctatcgttctgggcaccatcaccttgtggctgacatgggttggcggtgtgggctttacgctcttttccatcgcgattggccttgccatgttttacgagtggacggaactgtccgcgacacggcaaaccgccttttcgcgccttttcggctgggcgtggctgatcgttaccgggattctcctgatccttgaccgcggtgcattgctgacaatcgggtttcttgtcgccggttgcgccattttgcttgtcacacagtggaaaagcggtcgtggctggcctgctgccgggcttttctatgcgggtttttcggcgctttcactttcgctcttgcggggggatgagccgttcggttttaccactatcgtctttctcttcgccgtcgtgtggtctacggatattgcggcctatttcaacgggcgggccttgggcgggcctaagcttgcgccacgtttctcgcccaacaaaacgtggtcaggcgccatcggcggggctgcggctgcggttgcgggcgggcttcttgtcgcctcgcttgtggcggctccgggaggctggggcgtgccggtgctggcgctgctcctgtccatcgtttcccagattggcgatcttgccgaatcgtgggtgaaacgccagttcggtgcaaaggattccggccgtcttcttcccggccatggcggcgtactcgatcgcgttgacgggcttgtcgccgctgccgcacttttgtatctctttggtgcgatttttgccgagccagatgttccgtctgcaatcttctttagtttc

SEQ ID NO: 80 - DNA, 315 bp, Brucella suis
atgacgaagcgagtgccccttacgaaagaatttgaacgtgaggctgtacgacttgcggaaacgagcggtcgtacgcagaaggagattgctgcagatcttggtatcggcctgtcgacgctgacacggtggatttcccgcaaccgggatgcaaccattgatgatcccgaactgattgatgcaaacgaagatatggtggctgaactacggcgtcgactcataaactttggcaccatattcttgttgcgacgagctttacggctgcgaggtagttttccgggcgcttatcatatcgggttgcgatgcctcggaactgct

SEQ ID NO: 81 - DNA, 882 bp, Brucella suis
gtggtagtcatggcccaatccttgagcaatgcccaacaccgtatgcgccgccgcattgcgcgcgttttctatctcgtgggcctgtttctggctatgcttgtcatatgcctgccgggcttctggatcgtgctttcctccttgcgtccgccggtggaaatcatggccaagccgcctgtctggattccgcaggacatctcgctcgatgcctatcgcaccatgttctcaggagcaggggcgggcggcgtgccggtctgggattatttccgcaattccatcattgtttccgtcacctccaccgtggtgtcgctcatcatcggcatgtcgggcggctatgcctttgcccggttccgctttcgcggcaagtcggcaaccttcctgggcttcatgctcacacgctccgtgccgggcatcgccctgtcgttgccgctcttcatggtctattcgcgcattggcatcatcgatacgcatttcggcctgatccttacctatgtggcgctgaatattcccttcacgatctggctgatcgacggcttcttccggcaggtgccgaaagatctggccgaagccgcccagatcgacggctgcacgcgctggcaggccttctggcaggtggaatttccgcttgccggtcccggcatcgcatcggcgggcatctttgccttcctcacctgctggaacgaatatgcgctggcctcccagctcacccgttccgtcaattccaagacgcttccggtcggtcttcttgattatacggccgagttcaccatcgattggcgcggcatgtgcgcgcttgccgtggtgatgattgttccggcgctggcgctcaccttcgtcatccagaaacacctcgtttccggcctgactttcggcgcggttaaaggt

SEQ ID NO: 82 - DNA, 2025 bp, Brucella suis
ttgccatggcgtgcagccatccacgcgggcagcttccaccgggtcctgcggcatggcgagaagcccggccagaatgaggattgcaaagacggcggtggacgaccagacttcggcggtcaggatcgccaccagcgccagattgccatcgataagccaggggatcgccctgtcggtcagccccagcgactgaagcgcattattcaccaggccgatattgtcgttgaacatgaacttgaactggaagcccaccagaaccggcgagaacatcattggaaacattatgagcgtgcgcagggtgcgcttgccccatgtggccttgttgaccagaagcgccaatcccagccccagaagcatttccagattgagtgcgatggtcagaaaaaccaccgtgcgcagaaaggctgcccagaacacccagtcactcaggatgcgctcataattgcgcaggccgataaagcggaacagcgtttccgggcgcgtcaggcgaaacggcgtgaagctggaataaagcgaaagcgcaagaggaaagagcaccaccgcagccagaatgatgaaggccggaagcaacaacaggaccggcggggaaagtcttttaaacattatccatcccaaatactgggttcggcgcatgagctgccgaaacggcccgccaccccttgcggaaaagcgggcctgccgaaaatcggacggtcaaaggtcgccggcgtcttccagaatctgggtcgccttctgggccgcactgtcgagtgcttccttcgaggtcttgtcgcccagaatggcagcctgcaattcaggatagacggcgttggaaatctcgatccaggacggggtgctcggaaccgggaaagcattcttcgccgcttcctggaacacggtcagcacttccttcttgtaaggatcagatgcagcctgctcgatatcccattcccagaccttggtgcgggtgggcagcgtaccattggcggcttccaccttctgggaatcctcattggtcaggaaccagaccagcgaagcggcagcctccttggtggggcaggcttcggtgacggagaaaccgtgatggccagaccagccggtgcgcttgccggacgagcccttgggctgcacgacgatgccgacattgcccgccaccttcgacgacttcggatcgttgaaataggtggcccagcccggccagtccagatcgagcgccacggtgccggaagcaaagcccgcgccaagatcatcccagagataattcgtggtgccagccgggacggccttggccttgtagaggtttacgaaccagtcgagcgcgcgcacgcctgcatcggaattgaaggcaggcttgccgttcttgtcgagatattcaccaccctcggcaaccagcatttcatagaagcggccattgatcgcctcttccttgcctgcgaactgggtgccgtagaaattcggcggatcggcgaagaagatcgcctgatcggaaacttccttccaggtctttggcggtgccagatcgtagccgtatttttccttgaacgccttcttcctggcttcgtcctgatagaggctcttctggtaataaagggcggacacatcgaactgggcacgcggcagcatgaccagcttgccattgagcatggacgccttgatgaccgacggcacgaaagcgtcgatctcttcctttggcaagagcggtttcaggtccgtgtaaagatcgccatattgcggcgcgaaagaagaatggttggaaccgacgcaccagttgatcgagccggaagccatgtcggacttgatttccttgtcgagctcaaaatgattcttcttggaaagaatgtttacctttgcgcccgtcgccttctcccaatcggcaatgcgcgcataaagcgtttcatattgctgaccgccgatcagcttggcgttaacggtcacgccctcgaacttgccgggcagatccgccgcgtaagccggggcaattcccattgccagcaaagccgcgcccgccagaacatgcgatttgaaatcctgcattcgtctcctccttat

SEQ ID NO: 83 - DNA, 792 bp, Brucella suis
atgagggagccgaccttggaaactgacgatcgctatcgcgcgcccgcgctggacaaggggctggacatattggaattgctggcgagtgtggatggcgggctgacacaggccgagatcgccaagcatctcgaccgcagccccaacgaattttaccggatgctggaccggctggtgaagcgcggctatgtcaccaaactcgatggcgaccgttattcactgacgctgaagctgttcgggcttgcgcaactccatgcgccggtgcgcaggcttgcctccttcgccacgcccttcatgcgcgagcttgccgaccgttccaaacaggccaatcagcttgccgtgttcgaccgcggctcggtggtggtcatcgctcagcaggaagcaccggattactggggcatttccatccgcgtcggctcgcatatcagcctgttcgataccggctccggtcacgttctgctcgccttccgctcaccggaagagcgcgaaatgatgattgccgcccatgtgaaaagccgtgatgaagtcaatctggatcaggacttctatgaccgtctcgaccagatacgcgagcgcggatacgagatgatggcgagcgcacagatggcgggcgcctataatctttccgcgccgatcctcggcccggacggcacctgcattgcagccctcacctgcccctatatcacgctcgtcaatccgtcttcggcccctgatatcacccagacgatcagcctgttgcagaagacggtacgcgacctctccaagctggtgggcgcggatgtaggcgtgacagga

SEQ ID NO: 84 - DNA, 3000 bp, Brucella suis
atgatgactgacatgcgcattcagaacaaaggccgcgtcaacaaggccaagtccgttcgcttcaccttcaacggcaagacctattccggctttgaaggcgacacgctggcctccgcccttttggccaatggcgaacatctgacgggccgctcgttcaaatatcaccgcccgcgcggcattctttccgccggttccgaagagccgaacgcgctgatgggcgtctcgcgcggggcgggccggttcgagccgaacacgcgcgccaccgcgctggagctttatgacggcctgaaagccgagagccagaaccactggccgtcgctgaagcatgatgtgggtgcgatcaacgatgcgttctccatgttcttttcggcaggcttctactacaagaccttcatgtggccgaagagcttctggaacaaggtttacgagcctttcatccggggcgcggccggtcttggcaaatcgccatccgaacccgatcccgacacctatgccagccgttatgcctattgcgatgtgcttgtcgtgggggcggggccagccgggcttgcggcagcgcttgaagcggcgaaaagcggcgcgaaggttatgctttgcgacgaacaggcggaactgggcggttcgcttctgtcagagccggagccggtcatcaatggccgcgcaagctgggactggctggatgagacgctggccgcgctcgccgccatgcccaatgtcacgcttttgccgcgcaccacggcgattggctattatcaccagaacatgctgggcctttgccagcgcctgaccgatcacctgccgaacccgccagccaacgcgccgcgtgaacgcatgtggcgtgtgcgcgcaaagcaggtcgtgctggcgcagggtgcaattgaaaggccgctggtctttgccggcaacgaccgtccgggcgtgatgctggcgggggccgggcgtacctatctcaaccgctatggcgtgaaggttggccacaaggccgttattgtcacgtcgcatgatagcgcctggcttgccgccttcgatctggcggtcgcaggcgtcaaggttcctgccatcatcgatgtgcgtgagcatgttgcgggcagcctcgtcaatcgcgcgaagatgctgggcatagaaacgctgaccggctggaccgtaaccgatacgggcgggcgtcaccgggtttcctccgtgcgcgccaatccggtgcagggcggcgtggctggcgcgccgcgcaccatagaatgcgatgtggtgctgatgtgcggcggctggacgccaagcgtccatctcttttcgcacaccaagggccagcttgtctgggatgaggagcgccagatctacctaccgggcgcacgcacggaagaaagccgctgcgctggtgcgggcaatggtcatttcgaccttgaagcggcgcttcgtgaaggagcgcaaagcggtgccggtgtggcatccgatgccggttacaaggcaagcgcgcgcgaatatgcggtggccggtgatttcatctgcaatggcataagctgccgcgaattgccgacggatcgtgatccgggcaaggccaaggccttcatcgacttccagaacgatgtcaccgccaaggatatccgccttgccgtgcgcgaaggcttccgctccatcgagcatgtcaagcgctacaccacaaacggcatggcgaccgatcagggcaagacgtccaacatcaacggtctggcggttgcgtccgatgccttgaagcgccctgcgccgcaggtaggcctcaccacgttccgcccgccctatacgccgaccacttttggcgctttctgcggctataatcgcggaaaactgtttgaagtcacgcgcaagacgccgatcgacgcttgggccgaacagcatggcgcggctttcgagccggtgtcgctgtggcggcgcgcctggtatttcccgaagcccggcgaagacatgcatcaggccgtggcgcgtgaatgcagggcaacccgccagtcgctcggcatgttcgatgcctccacattgggcaagatcgaggtggtcggcccggatgcagccgaattcatgaaccgcatgtataccaatccatggacgaagcttggtgtcgggcgctgccgctatggcctgctgctcggcgaagatggcttcatccgcgatgacggtgtggtcggtcgcctgacgcaggaccgtttccacgtgacgaccacgaccggcggtgcggcacgcgtcctcaacatgatggaagactatctccagactgaatggccgcagttgaaagttgcgctcacatccaccaccgaacaatgggcggtggtggcgatcaatggcccgaacgcgcgcaagcttatcgagccgatggtcgaggggctggacatttcggacgaggctttcccgcatatgtcggttgccgaatgcacattcctcggcgtgcctgcgcgtctcttccgcatgagcttcaccggcgaactcggcttcgagatcaacgtgccgtcgcgctatggtcttgcgctgtggaaggcgctttacgaggcgggccagcaatatgacatcacaccttacggcaccgagacgatgcatattctgcgcgccgaaaagggctatatcattgtcggccaggatacggatggcacggtgacgccggacgatgccagccttggctgggccattggcaagcagaagccggatttcgtcggcaagcggtcgctgtcgcgtcccgacatgctgaaaaaggaccgcaagcatctggtcgggcttctgaccaaggacccgaagctggtgctggaggaaggcgcgcagatcgtggccgatccaaaacaggcggtgccgatgaccatgcttggccacgtcacgtcttcctattggagcgagacgcttggccgttccattgccatggcgctggtgtccggtggcaaggaccgtatgggcgagacaatatatatgccgatgccggatggcagtgtgcatgaggccatcatttcgggaaccgtcttctacgatcccgaaggtaagaagttgaatgca

SEQ ID NO: 85 - DNA, 882 bp, Brucella suis
ttgcgttctcttgcccagcggatcgctgacaaggctttttcccgccattgcgccgatccagtccagcccggcacgcaagcgcaccacctcacccacaaccacaatggctggcggttgcagcccggctgcttccacctcgattgcagcccggcccaacgttgtttcaagcacggtctggtccggcaggctggcattgcagacgaaggccaccggctcatctggcgaacgtccagcctcgatcagccgcccggctatgaggccgatatgtttcatcgccatatacatgacgatgaccggcgacgatcgggcaatacccgcccaatcgacgccatcgggcatggcgccggttgcgtcatgcccggtcagaaacgttaccgcctgattgatatcgcgatgcgtggcggcaattcccgcataggcaagcccgccaaccccggctgtgacccccggcacgatgcgaaacggaacatgatactcaacaagcgtgagcgcctcttccgccccgcgcccgaacacgaaaggatcgccacccttgagccgcagcaccttcttgccttgccgtgcaagctccaccagccgcagcgagatgtcgcgttgcctgggcgatggtttgccgccgcgcttgccggcatattccaacgtggcggtcgcattcttcagcgaaagacaggaatcatcgaccagcgcgtcatagacgatcacatccgcctgttgcaaagcgttcaccgcatggagggtcaaaagaccgggatcgcccggaccggccccgacaagccagacatggccgggctccatcacgggcaaagccactttgcttttcgcgatcacacaggcagccgcttcgtcagctttgctcatactctacaaccacgttcaatttac

SEQ ID NO: 86 - DNA, 825 bp, Brucella suis
atggtaacctatatcgaagcgacaagcgcacccgtgaaatatacaggccagatcaggctctacggccccgaagccttcgaggcgatgcgcaaggtctgcaacctgacggcacgttgccttgatgcgctgaacgatatcgtcaaacccggcgtgaccaccaatgagatcgaccgtttcgtatttgaattcggcatggaccacgatgcgtttccggcaacgctgaattatcgcggctacacgaaatcgacctgcacatcgatcaaccatgtggtctgccacggcatcccgaacgacaagcctctgcgcgaaggcgatatcgtcaatatcgacgtgacttatctgcttgacggctggcacggagattccagccgcatgtatgcggttggtgaaatcaagcgcgcttccgaacgtctgctggaagtgacctatgaaagccttcttcgcggtatcgccgccgtcaagccgggtgcgaagaccggcgccatcggcgcggcgatccagacctatgccgaaagcgaacgctgctcggtggtgcgcgatttctgcggtcatggcgtcggccggcttttccatgatgcgccgaatatcctgcactacggcacccccaatgaaggtgtcgagatcaaggaaggcatgatcttcacgatcgagccgatgatcaatctgggcaaaccgcatgtgaaggttcttgccgatggctggacggcggtgacgcgggaccgctccttgaccgcacaatatgaacacacggtcggcgtcaccagggatggctgcgagattttcacgctttctccggccaatatctttggcccgccttccatgcgg

SEQ ID NO: 87 - DNA, 465 bp, Brucella suis
atgtacggacgcgcccgttccggagcccgtttcctagctatcgccatgttggccccactggctgccgcctgcacgacgactgtgccgacatccaccgccacgccgggcaagactgaagctcccgtcagcgcaccgaccatttcgctgccgcgttttacgccgacgtcttatgtcgatccggcgctggcgcagccctgcatcactgccgcagccaacaaatatttcctgccggagcgcgttatcagcgccgtcaattcccgtccgggtgcaggtggcggcatggatgtggatctgaaggtcgatctgcgcacggcagtttgccatttgaattcaagcggcggtgtgcgtgctgttatcgatacttcgccaaagagcgcggatcagattgcagccgaagaagctgcggccaaggccgatgccgccgcagccgcccagccgaaaaaggccgcggcaaagaaaaagaag

SEQ ID NO: 88 - DNA, 897 bp, Brucella suis
ttgaccggccatatcaagattttcggccaattcgccaaaatgaaattcgggctggtaaggcgcgatctgttccagaagccggtcgatcagctctccagccatgatccagggaaaagccgggatatcataaatgggcttatctggataaaggacggcacattagccgcccggacgatccagcgcgtctatcaaataacatttgaagccgaaaagcccaagctggaaaacggaaaaaagcgcaacaggcccggcaccgataatggctatcccggtttgccttccggcctccattacgccctcctcaatcgtgctgcccagcctgcccggtcagttctggcgttcgggcacctgaacgacaagcccctccagatcgtcggtaacgcggatctggcaggaaaggcgcgaggtcgggcggacttcataggcgaaatccagcatatcctcttccattggatccggcccgccgaccgtatcggcccagtcgtcatcgacatagacatggcaggttgcgcaggcgcaagcgccaccgcattcggcatcaataccgggaatgccgttgcgaatggccgcttccataacgctggacccgttgtcggcctcgacctcggttcgcgtggcgccatcggcggatacgaatacaatcttcgtcatttttattccttgagacacgtcttcgacgcgatctttgtgctacgcgcccctggcccgatcctcaggctgatcgcgcgattgatcccgaaatggcagatacggccttatgaatggagtttcaaccaccgtaaatggcggctccccgactgcgccaatatgcccccgccctgtcaaaatcggcaaaacggaacgccggagccaatcccaccttgccgcagggggaaaaagcgccatattatcggctca

SEQ ID NO: 89 - DNA, 216 bp, Brucella suis
atgttgcgctatgtcggcaactgcccgccccgccgccagcatcagcgcgaaaccgtccttgatgccgccctccatcgtcaggcggtcagcccgcgccatttcttccggcgtaagcaattcaaacataacgcacctttctccagcggctttctgcctatgattaattacgcgctgccggactttatgcaagtttgcttgtttttaccgcaagttgca

SEQ ID NO: 90 - DNA, 1407 bp, Brucella suis
atgacgactgccaacgatattctgaaacagatcaaagataacgacgtcaagttcgtcgaccttcgcttcaccgatccgaagggcaagctccatcacgttacgatggatatcggcctcgtggacgaagacatgttcgtcgacggtgtcatgttcgacggctcttcgatcgcaggctggaaggccatcaacgaatccgacatggttttgatgcccgatccggaaacggctcatatcgacccgttcttcgcccagtccacgcttgtcattctctgcgatatccttgagccgctttccggcgaaagctacagccgtgatccgcgtacgactgccaagaaggccgaagcctatctgaaatcgctcggcattggcgatacggtctatgtcggcccggaagccgaattcttcgtcttcgacgatgtgaagttcaaggccgatccgttcaatacgggcttcaagctcgatagcgccgaattgccatccaacgacgacaccgattatgaaaccggcaatctcggccatcgtccgcgcgtcaagggcggctatttccccgttccgccaatcgacagcttgcaggacatgcgttccgaaatgctgaccgtgctctcggaaatgggtgtgacggttgaaaagcaccaccatgaagtcgcttccgcgcagcacgaactgggcacgaaattcgacacgctggtgcgcaacgccgacaagatgcagattcagaaatatgtcgtgcatcaggttgccaatgcctatggcaaaacggcaaccttcatgccgaagccggtctttggcgacaatggctccggcatgcatgttcacttctcgatctggaaagacggcaagccgaccttcgcaggcaatgaatatgctggtctgtcggaaaactgcctctacttcattggcggcgtcatcaagcacgcaaaagctgtgaatgccttcaccaacccgtccaccaactcctacaagcgtctggtgccgggctatgaagcgcctgttctgctggcctattccgcacgcaaccgttcggcttcctgccgcattccattcggctcctcgccgaaatcgaagcgtctcgaagtccgcttcccggatccgtccgccaacccttatctctgcttctcggcccttctgatggcgggccttgacggcatcaagaacaagatccatccgggccaggccatggacaaggatctctacgacctgccgccgaaggaactgaagcagattccgaccgtctgcggctccctgcgtgaagctctacagtcgctcgacaaggatcgtgaattcctcaaggctggcggcgtgttctccgacgaccagatcgacagcttcatcgagctgaaaatggctgaagtgatgcgttttgaaatgacgccgcacccggtcgagttcgatatgtatkactcggta

SEQ ID NO: 91 - DNA, 132 bp, Brucella suis
gtgaagattccggcggttggactgcaattctttcaggctgcggcaggcttgcccgataagggcgttaaattatcattttatagaaatgtgcctagcaatcgcgcgggcgcgcgtctattgacagggtttgat

SEQ ID NO: 92 - DNA, 1332 bp, Brucella suis
atggccgcaacattcgatacgattttgaaaggcgcaaccatcgtcaatcatgacggtatcggccagcgcgacattggcattcgtaacggacgcatcgccgctatcgggtcgcttgcagggaactcagccggtgaagtgattgactgcactggcctgcatatcctacccggcgttgtggatagccaggttcatttccgcgagccgggtcttgagcacaaggaagatctggaaaccgggtcacttgccgccgtgctgggcggcgtgacggccgtttttgaaatgccgaacaccaagccgctgaccacttcggccgaaatgctggaagacaagattcgccgtggccgccatcggatgcattgcgatttcgccttctgggtcggcggcacgcgcgataatgcgaaggacgtggccgaactggagcgcctgccgggcgcggcgggcatcaaggtattcatgggttcgtccaccggcgatcttctggttgaggacgatgatggcgtacgctcgatcctgaggaatacgcgccgccgcgcggcttttcattccgaagacgaatttcggctcaaggaacgtgaaggattgcgcgttgagggtgatccatcgagccatccggtatggcgcgacgagattgccgcgcttcaatgcacgcagcgcctggtacgcatcgcccgcgagactggcgcgcgcattcatgtgctgcatatttcgaccgccgaggaaatcgacttcctgaaagattacaaggacgtcgccacctgcgaggcgacgccgcatcacctcaccctgtcggcggatgattacaaggcgcttggcaatctcatccagatgaacccgccggtgcgtgacaagcgccaccgtgaaggtgtgtggaaggggatcgatcagggtatcgtggatgtgctgggttcagaccatgccccgcacacgctggaagaaaagcagaagtcttatccggcttccccatcgggcatgaccggcgtgcagacgctggtgccgatcatgctggaccatgtgaatgccggtaagctttcactggagcgttttgtcgatctttccagccacggcccgaaccgcattttcggcatggcgcgcaaggggcgcatcgcggtcggctacgatgccgatctgacgatcgtggacatgaaacgccgcgaaacgatcacgcatgaacaggcaggatcgaaagccggctggacgccctatcatggcaagaccgtcaccggatggccggttggcactttcgtgcgcggcatcaaggtgatgtgggaggcggaaatcgtcaatcctcacaagggcgagccggtggagttcctcgaagcgctgccccgtcgc

SEQ ID NO: 93 - DNA, 1782 bp, Brucella suis
atgggatgtggtagggtaatcgcgggggagaacaatgatggcacgcgagcgcatttatatctacgacacgacctcggtggatgggcagcagacgttacccgcccgcccgcagccgcgaagcgagcgaggacgggcaaaggcaaaagcgggcgtttgcctgggagggcaataatggcacgcgagcgcatttatatttacgatacgaccctgcgggatgggcagcaaacgcccggcgttgacttctctgtggaggacaaaaagtcgattgtcacgctgttggaagagctgggcgtcgattatgtggaaggcggttatccgggggccaacccgactgatacggctttcttcaagcgtcgccagaccgctcgcgccaagttcgtggctttcgggatgacaaagcgggcgggcgtttccgtttccaacgatcccggcctgtcgagcttgttgcaggcttctgccgatgcggtctgctttgtcgccaagagctgggattatcatgtgcgtctggcgctcggctgcaccaatgaggaaaatctggaatccatcacggcttcggtaacggcggcgcgcatggctggccgtgaggcgctggtcgattgcgaacatttcttcgatggatacaaggcgaacccggaatatgcgctggcctgcgcgaaggctgcctattcggcgggtgcgcgctgggtggtgctgtgcgataccaacggcggcacgcagcctctggaagtggcggcaatcgttgaagcggtgaaggcggttgttcccggtgagagccttgggatccatgcccataacgataccgaacaggctgttgcggtttcccttgctgcaatcgatgcgggggtgcgtcatgtgcagggcaccttgaacggcattggcgagcgttgcggcaatgccaatctcgtttcactcatcccgacgctggttctcaaaccctattgggcggagcggttcgagcttggcatcaagcctgcatcgctgaaatccatcacgcgcatttcgcgcgctttcgatgatatcctgaaccgcccggccacggagcaggcaccctatacgggcacttcggcctttgccacgaaggctggcatccacgcttcggcgctgatgaaagagccgcagacctacgagcatattccgccggaatcggttggcaatagccgccgtgtcatggtgtcggatcagggcggcaagtcgaatttcattgccgagctggagcgccggggtatccgcgttgccaaggacgatgttcggctcgatacgctgattgcgctggtcaaggagcgtgaggccgagggttatgcctatgagggtgcggacgcaagttttgagcttctggcacgcggtatgctcaatccgcaaccggagttcttcaaggttgaatcgttccgctgcctcgtggagcgccggtttgatgccaacggcatgttgaaaaccgtgtccgaggcggtggtcaaggtcgaggtggatggcgagatgcatatgtcggtcgcggagggccatggccctgtcaacgcgctcgatatcgcattgcgcaaggatctgggccgctttcaggccgagatcgaggatctcgaactggtcgatttcaaggtgcgtattctcaacggcggcacggcggcgatcacccgcgttctgatcgaatcgggtgatgccacgggtgcgcgctggcgcacggtgggcgtttccgacaatatcatcgatgcttcctttcaggctttgatggattccgtcaattacaagctgatgaaaaaccgggccatggcaggtctggccgctgccgaa

SEQ ID NO: 94 - DNA, 1572 bp, Brucella suis
atggcaattgcaccaaaggcgggttttgctcgcacccttttcgcaaccgttgcactgggggcgatgagtgtcgcgggcactgtgtcgatgggaacgccgcccgcgctggctgcgcaaggtcccgcttcggtggccgatcttgccgaagggcttctggatgcagtcgtgaatatctccacatcgcagaccgtgaaggacgatggcgagggggacggcccggtgccgatgccgcaggtgccggaaggttcgcccttccaggaattcttcaaagacttcttcaatgacaaggatggcgcgcagggcgatgattcacgcaaggtgcagtcgctgggttcgggtttcatcatcgatgccgaaaagggttacatcgtcaccaacaatcacgtcatcgccgatgccgacgagattgaggtgaatttcaacgatggttccaagctcaaggcggagcttgttggcaaggatacgaagaccgatcttgcgatcctgaaagtcgacccttccaagcataagctcaaggcggtgcatttcggcaattcggaaaaggcgcgcattggcgattgggttctggcgatcggcaatccgttcggccttggcggtactgtcacggcaggcatcatttcagcacgcaagcgcgatatcaattccggcccctatgacgatttcattcagacggatgccgcgatcaatcgcggcaattccggcgggccgctgtttgatatggacggcaaggtgatcggcatcaatacggcgatcatttcgccttctggcgggtcgatcggcattggttttgctattccggcggaaatggccgccggtgtcatcgatcagttgaaggaatttggcgaagtgcgccgcggctggcttggcgtccgtcttcagccggtgacggaagacattgcccagagcctcgggctgaaagagaccaagggcgcgctgattgccggtctcatcgaaaattccggtgtcgataacaaggcgatagaggcgggcgatgtggtgatccgctttgacggcaaaccggtggatacggcgcgcgatcttccgcgcctcgtggcggaaagcccggttggcaaggaagtggaaatcgtcgttatccgtcagggcgcggaaaagaccctgaaggtcaagcttggccgtctggtcgaggacgacaagagcacggagccggcggttgaagatcaggtgcccgcgccggatgacggcgaacagccgggagcccggcaggagacgccggacaagtcggacaagccgaagaaggaacacaagagcgatgctgctccaagcgttctcggcatgaagctcagcaagctcgatgatgatattcgcggcgagtttggcattgccgaagatgtggagggcgttgccatcctctatgttgcaccggggacggctgccggcgaaaagcgcatcgaaacgggcgatgtcattgtcgatatcggtcaggtgacggtgaagacgccggaagatgtgaaaaagcgtatcgatgcactgcgccgcgaggggcgcaagaatgcgcttttgatgctggcttcgcgttccggggagttgcgtttcgtgacgatccgtatcgac

SEQ ID NO: 95 - DNA, 450 bp, Brucella suis
atgtgcttgggaaagggggctggcttagcggacagcgccttcaaaagcgttcggcatggaagtgttcttgaggtattcaagagccttctttgccgatgcaaccagcgcagcgaatgcttccggttcgtggattgcgatatcggaaagaaccttgcggtcgatttcgatgccagccttggccagaccgtcgatgaaacggccataggtcagtccctgctcgcggacagcagcgttgatgcgctgaatccagagggcgcggaacgaacgcttgcggttcttgcggtcgcggtaagcatactgcttggaacggtcaaccgcagccttggcggtgcggatggtgttcttgcgacggccacggaaaccggcagcctgatccagaaccttcttatgctttgcgtgggcggtaacgccgcgttttacgcgtgccatgttatgatctccttctcagac

SEQ ID NO: 96 - DNA, 1320 bp, Brucella suis
atgagtattcttggtggtcatggagcatcacaacacggaaccgcagggcgcaaggtgcgcctgttaacggttctggacgttgggtcgagcaaggtttcctgtgtcatcgcaaggctgcgcccgcatgaaggcggcgcgttgctgccgggacgcacccatcgcatggaagtgctgggcatcggccatcagcgttcgcgcggcgtgaaatccggcgtcattatcgaccttgatgcggccgagcagtcgatccgccttgcggtagatgcggccgagcgcatggctggcctgaccgtcgatagcctcatcgtgaatatttcggctggccgtctcaaaagcgaaacctttaccgcaagcgtcaatcttggcggccatgaagtcgaacagacagatatccgccgcgttctggctgctggtgcaaaacaggcgctggctgccgaacgtcacctcgttcattcgctccccgtcggttatacgctcgacggcgagcgtggcatccgtgagccgcttggaatgctgggcgacagccttggcgtggatatgcatgtgctgacggctgatgcggccccgcttcgcaatctggagctttgcatcaaccgctgccacctttcggtggaggcaatcgttgcaacgccttatgcaagtggccttgctgccctggtgggcgacgaggcggaaatgggtgcggcctgtatcgacatgggcggcggcacgacgacgatttccgttttttcggaaggcaagttcattcatgccgatgcggttgccatcggcggcaatcacgtcacgatggatgtggcgcgtggcttttccacccgtatggaagacgccgaacggctgaaggtcatgtatggctccgccctgccaagtgctgccgacgaccgcgatctgatctccgttccgcccatcggcgatgatgagcgggatgtacccaatcaatatccgcgctcggtgctgacgcggattatccgtgcaagagtggaagagacgctggaactggtgcgcgacaggctgaaccagtcgggcctcggacatatcgtcggcaagcgcgtggttctgacgggcggcgcaagccagcttcccggtatgcccgaagcggccaggcgaatccttgcaagaaatgtacgaatcgggcgtcctctaggaatagcgggattgcccgaggcggctaagggggcagctttcgcggcgaccgtaggccttctgatttacccgcaggtggcggggatagaagagcgttcggtcaaagcagcgtcctccggtctgatgaccggtacgggtgggcgcattcaacgagtcggccaatggctgagagagagtttt

SEQ ID NO: 97 - DNA, 135 bp, Brucella suis
atggcggcagacgcgccacatgatatagccaataaagagcggatagagcattttcgagccaaaagtgtgaaacggtttgcgttggaaaatgcgacaaaacaaatagttagagcggttccggcgattctgttaaaa

SEQ ID NO: 98 - DNA, 1821 bp, Brucella suis
atgcggctgagattgttttcggcaaagaaaaaggcgcctgatggctccattcacggtgaagtgccggccggtgatgaaaagctggccggtaatatggcgtttgtcggctcgcgcaagcgccatggcaaccgcgcgcgcaaccggctctggatggccattgcctgtttcgccggcatttatggcgtgatggctggcaagcttgtctatttcggcatgatcggtggcgtggatcaagatgccgccggtccgtttgtgcaccagcttgcatcacgccccgacattcttgaccgcaatggcgaaattctcgcaaccgatatcaagaccgcatcgctctatgcggagccgcgcaagatcgtggacccggacgaaaccatcgaaatgctgtcgactgttttgccggatctcgactgggaggcgacctataagcgcctgaagagtggggccggtttcgtctggatcaagcgcgggctgacgccgaagcagcaaagccagatcatggcgctcggtgtgccgggcattggcttccgcacggaaaaacgccgcttttatcccggtggcccgaccgcttcgcatattctcggtctcgtcaatgtcgataatcagggcattgcgggcatggagaaatatatcgatagccaggggctgagcgatctgcgctccgtcggccttgcaacggggcagtcgctggagccggtgcatctttccatcgatattcgcgtgcagcacatcatgcgcgatgttctggtcaaggccatggagcgctaccgggcaatcgcggctggcgctgtggtgctgaacgtcaagacgggcgaagtgatcgccatgtcgtccgtaccggatttcgacccgaataatccggtgcacgcgctggacaaggatcgcctcaaccgcatgtcggcaggcacttatgaaatgggctcgaccatcaagagcttcacgacggccatggcgctcgattccggcaagttcaccttgcagtcgaagctcgacgcctcgcgtccgctggtgttcggccgccagaccatccgcgacttccacggcaagggccgctggctcaccttgccggaggttttcatcttctcgtccaatatcggttcaggccgtgaagcggacgccgtcggcatcgaagggcatcgcgccttcctcaagaagatcggccttctcgaccgtatgcagacggaattgccggaagtggcgcgccccgttgagccgcgcgtgtggaaaaaggttcattccatgaccatttccttcggtcacggcatgatgaccacgccgcttcagacggctgtgggggctgccgctttgatgaatggcggcaagctgatcgagccgactttcctgccgcgtacggaagcgcaggcggctcgggccagcaagcaggtgatccatccacaggtttctgccgatatgcgctatctctaccggctgaactcaactgcccctggcggttcgggccggcgcgcgacggttccgggctaccgtgtcggtggcaagaccggcacggcggaaaaggtgatcaatggccgctattccaaggatgtgcgcttcaacgctttcctggcgtcattcccgatggataatcccacctatgtcgtgcttaccatcattgatgagccgaagccggaagagggcaagttcagcgccaccgccggtcttaatgcggcgcccatggtggctgaaatcattcgccgttccgcatcatttcttggggtcagtcccgatttccgcaaggaagctttgcctgcttcggtggaaaacgtcagcggcaggcccgattttcaacaggaagtgccgccagcaatggtttcaaacgaatacgat

SEQ ID NO: 99 - DNA, 576 bp, Brucella suis
gtggcgcgtccgcccaatcttgcgatcttcgatatcaagatgccgcgcatggacggtatggagcttctgcgccgcctgcgccagaagtccgatctgccggtcatcttcctcacttccaaggacgatgagatcgatgaactcttcggcctcaagatgggtgcggacgatttcatcaccaagcctttttcgcagcgtcttctggtcgagcgtgtcaaggctgttctgcgccgcgtggcggcgcgcgacggcacggcaaagcccgccggccagcaggccaagtcccttgagcgcggacagctcgtcatggatcaggagcgccatacctgcacctggaagggtgagccggttacgctgactgttaccgaatttctcatccttcactcacttgcccagcgccccggcgtggtcaaaagccgtgacgccctgatggatgcggcctatgatgaacaggtctatgtggatgatcgcaccatcgacagccacatcaagcgtctgcgcaagaagttcaaggcggttgacgacagcttcgagatgatcgaaacgctctacggcgtcggttatcgtttccgggaagcg

SEQ ID NO: 100 - DNA, 1164 bp, Brucella suis
atgttcttcgcggtgctgggcgctgcggtgggcattacgtggaccgtgcaggtgcttcagcgtatcaactttctgaccacaagcgggcaaaccttccagacaatcgtccagttcagttcactgctgattccgtctgcgattccgcttgtgatgccctttgcgctcatcattgcaattacccagacgctttcgacgatgaatcaggattcggagctcgtcgtcatcaacgcttccggcgcgccgcgcagcgcggtaatgcgcccgattctgatcctggctgcaacagtttccgtgacttcgtttctgatcgccaattatgtcgagccctatgcccgcataaatatgcgcgcgatgatcgccaatgccagcgccgacctgatgcatgtcatcattcaggaaggcactttccgcaagcttgccgacaatctctatattcaggttgccgaacgccgggcagacggcagcataggtggccttttcattgcggattcgcgcgatccgacactcgatctcatttattatgcggccgacggcgccattgcctccacgccatcgggcgacatgctgctgatggaaaatggcgaagtgcagcgccgcaatgtctcggacggaaccgtttcgatcatcaagttcgattcctatgccttcgacctcaaccagtttgcctcagcaggtgatgatttcgttatttttgccaaagacaggccgcttacctatctactgaatcccgaccccaacgacccgatgcttcagacaaggcctttgacttacaaagccgaattgcatcggcgtttcacacagtggatctatccgctcattttcgcgatgatcgcgctcgccattgccggcgattcccgctcgcatcgcgaagctcgcgtttcagcctctttttccgccatcagcacggcgcttctcgtctattgggctggctatttcgcctccgaccgcgcagacaaggacccaagctacattctcgtcatgtatctggtgccgctggtcgtctttctggttacgggcttcatattgctgacagggcggcgcctcggcctgcccgataagtggagcgatcgcattctcgatttcctcgacaatacgaggcggcgccttggcgaattctacgcccgctttaccggacgtccccgcaacaatgccggaggccggtta

SEQ ID NO: 101 - DNA, 2106 bp, Brucella suis
atgggaaacgcccgacattgcaggaattaccgaataatggccacccgcaagacaaccaccggcgagcaaacgcatctgccgcccgatgacgaacaggacgttgcgggcatcattatcggtgaggcagaaaccgaagacgatgacgatgcggcggcaaacggcgaggaagagcctgcccgcatagcttcggctgcggaatccatccagtgggattcgtcggacgggctgcccgatacggcggggctcagcggcccggacatcatcaatgcctttatgaagcgcctgcccaacaatcccggtgtctatcgcatgttcaacagcgatggagacgtgctttatgtcggcaaggcgcgcaacctcaagaagcgcgtttccaattatgcgcgcggcattggccattccaatcgcatcacacgcatgatccgtgaaaccgtgacgatggaattcgtcgtcacgcgcacggaaacggaagcacttctgcttgaagcaaatctcatcaaaagatcgcggccacgcttcaacgtgctgatgcgcgacgacaagtcgtttccgtatattctgctgaccggcggccaccgcgcgccgggcattttcaagcatcgcggcgcacgttcgcgcaagggcgactatttcggcccctttgcttcggcgggggctgtgggccgcaccatcaacgcgctccagcgcgcttttttactgcgcacctgcaccgattccgtctttgaaacccgcacacggccctgcctgctctatcagatcaaacgctgctctgcgccctgcacctatgaaatcagcgacgaggactatgccgggcttgtggcagaggccaaggcctttctttccggcaagagccagtcggtcaaggatcatctggcagcggcaatgcaggcagcttccgccgatctcgattttgaacatgccgcagtctatcgcgaccgtctcgccgcgctttcacacgtccagtcccatcagggtatcaatccgcaaacggtcgaggaagccgatgttttcgccatccatcaggaaggcggcatgacctgcattcaggtcttcttcttccgcaccggccagaactggggcaaccgcgcctattttccgaaagccgacagttcgctcgggccagcggaagtgctgggcgctttcctgtcgcagttctatgatgacaagccttgcccgaaactcgttctcctttcggaaacggtcgaagaacaatcccttatcaccgaggcgctctccacgcgcgccggtcataaggtccaggtctcggtgccgcagcgcggtgaaaagaaagaactcgtgcagcacgcgctgaccaatgcgcgcgaagcacttggccgcaggctggccgaaacctcatcgcaggcgcgcttgctacaggggcttgccgaaacattcggcctgccgcgcgcaccgcgccgcatcgaggtttacgacaattcccacatcatgggcaccaatgcggttggcggcatgatcgtcgccgggccggaaggctttgtgaaaaaccagtatcgcaagttcaatatccgttcgaccgacattacgccgggcgatgatttcggcatgatgcgggaggtgatcgagcggcgcttctcacgtctggtaaaggaacatggaacccctgccggcgaggtggagaacccggacgccttccctgcctggcctgatgtcattctcattgatggtggccagggacaggtgggcgcggtgcgccagatactcggcgagatgggcatcagcgatctggtcacggcaatcggcatcgccaagggcgttgaccgcgaggcaggccgcgagcggtttttcatggaaggcaagcagccatttaccctgcccccgcgcgatcccgttctttatttcatccaacgcctgcgcgacgaggcgcatcgctttgccataggcacccaccgcgcacgacgcaagaaggaaatggtcaggaatccgctggatgaaattgccggcataggccctacgcgcaagcgcgcccttttacatcattttggaacggcaaaagcggtttcgcgcgccgctgtcgaagatctgatgcaaatcgatggtatttcggaagccatggcgcgggcgatccacgaccacttccgggacaaa

SEQ ID NO: 102 - DNA, 1062 bp, Brucella suis
gtgactttatcagggcaagttccggttcgggaagttgaggtcgttgcacccaacttcaagcgccgcctttccggcgtgacctcgacgattgttcagctcattccattgcagcgcgcaatggggctgaaaattgccactatggggccgggcctgcctgacactctcccgcatcttggatggagcgcgttgccctccttctggtcgcgacctaaaaccagacggtttcgcatctggcatgcgcgccgcaacattgaaatgcttgccggaattttcatgcgggatgtcctgcgcatgaaactgcggctcgtttttacctcggccgcccagcgccatcacaaacctttcaccaaatggctcatccgccgcatgaatgcggtgatcgcgacaagtgtgcgttcgggaagttttctcgaagtcccgcatcaggtcatcatgcatggtgtcgatctggaacggtttcacccgcctcttgcggaggacgatgatttttccgcttccggcctgccgggtaaatatgccgtggggtgttttggacgggttcggccttcaaaaggaacggaccttttcgttgatgcgatgatcgcgctcctgccgaaatatcctgactggacggccattgtcacggggcgcacaaccgccgaatatcaggcctttgaagccgagctgcgcaccaggattgcggcagccggtttgcaggaccgtattcttattctgggcgaagtgccggacgtgcgcgtctggtatcgccggctcacgctttacgtcgcgccttcccgcaatgaaggctttggcctgacgccgcttgaagccatggcctcgaaaacagccgtcgttgccagtgatgccggggcttatgcggaaatggtcgttgaggatacgggccgctttgtgccagccggtgacgggcgcgcattgacgaacgccatcgaaccttatctggccgatccggccatgacaaaacgctgcggcgaaaatgctctggcgcatgttcgcgaagcctttccgctccaaaaggaagcggctgctatcagcagcgtttatgagcaggttttcgcaggaaga

SEQ ID NO: 103 - DNA, 546 bp, Brucella suis
atggccgcgcattatgcaggcattccgtttgaaagcgtcatcgtcaacacatccgcggagccggaaacgctcattcaggtcaaccctttgggcaagattccaacgcttgtgaccgatgacggcaaggccgttttcgatagccgcgccatcacgcaatatctcaaccgcgtttcgggcaacaagctttttccacgcaatgccgaaaagcgcaccgatgcggaacgctatgaagcaatcgccgatgggctttgcgacgttttgctggcccatgtctatgaacggcggatgcgcccggaagagaaaattcatcaaccctggctcgacctgcaatggcgtaaggccgaacgtacgcttgatcttctcaacgaggccccgccgcgccttgccggaaagcttcatgcgggacatctcgcggttgccgcagcactcggctatctggcgttgcgctttgaaggcaaatgggaacgcaaccgctcgaagctgaaacgctggatgaagcgctttcaggaattgcacccggagctggtcgagcttctgccgcgcggc

SEQ ID NO: 104 - DNA, 1296 bp, Brucella suis
atgcccaaggggctttatgcgcgctcgctcatcatcattatcgcgcccatggtgctgctccagtcggtcattgcctatgtgttcatggagcgccactggcagatggtgacggaacgcctttcgacggcagtggttcgcgatatcgcagcaatcatcgatcttcttgaaacctatccgcaggagccgggttacgacaatctgatccgcatcgcgcagcagcggctggcgttgaacatttccatcctgccccccgatccactgccgccgccggggccgaaacccttcttcgccattctcgactatttcctgagcgaagagatcacccggcagatcaaccgccctttctggatcgatactgtgggtgattccgatctgatcgaaatccgcatcaagcttgaagacaaggtattgcgcgtttttgcccggcgcagccaggcctatgcgtccaataccggcatcttcctcacatggatggtgggcacggcgctggttctgcttatcattgccatcgccttcctgcgcaaccagatcaagcctatccagcaattggcgcaggccgccgaaagcttcggcaaggggcggcccatgccggaaggttttcacccgcgcggggcggaggaagtgcgccgcgctggcctcgcctttatccagatgcgctcgcgtatcgaacgccagatcgagcagcgcaccgccatgctttccggtgtcagccacgacctgcgtaccattctcacccgcttcaagctgcaactggccctcgccggccattccatcgataccgagccgctcaatcaggatattgccgacatgcagaccatgctggaggggtatctggcctttgcgcgcggtgagggagcagaagaaaccgcgactttcgacgtcacccggctttgcgaaaaactggaaagcgaagcgcgcctgcgcgagcgcggtttcaagtacacgatagagggcgaccgcgaaatcaacgtgcgcccgaacgccttctcacgacttgtcagcaatctggtttcaaacgccttccgtcacgcgcagaatgtcgagctttcgatttcgcatgtggacggctggcttaaagtcatcgtggacgatgatggaccgggcatacccgaagatcgcagggaggacgtcttcaagcctttcgtgcggcttgatgaggcacgaacgcaggattcaggcggcaccggcgggcttggccttgcgattgcccgcgacatcgcacgcagccatggcggcgacattgcgctggaggattcgccgctcggcggcctgcgcgccaccattcatattccggct

SEQ ID NO: 105 - DNA, 435 bp, Brucella suis
atgttctcaggacatggcatggccgggccgggccgggccgggccggagcatccatcatggaacaggcctcccgctcaagcggcctcgcctctattttccacaatcgaaacgcgcaaaatccggcgcaagcccgaagccgcaccggaaagcaaaaaggcgccgctaccacagcgcctatatcacagccaatcagcgagcgcctgtacggccgccgatgagaccttcgcgctgggcgcgcttacgggccagcttgcgtgcgcggcgaacggcttctgccttttcgcgggcgcgcttttcggacggcttttcataatggccacgcatcttcatttcgcggaagataccttcgcgctgcatctttttcttgagagcgcggagagcctgatcaacattattatcgcggacgagtacctgcacgtgtattcccgtatcggt

SEQ ID NO: 106 - DNA, 450 bp, Brucella suis
ttgctctggatcacgctcgtcagcgaactgatattctcgctcgatccctgtgccctatcgacagtgctgagcgtcgaattggtactttggcacccggcgacggtcaaagccagaagtgcggcggcgatgataaggccgtttcggcctgctccacccgacgcctcaattcccgaaacctccgtttcagacaattgcatcacctttaagcgcatcgccaattgcagcattcctttcagctcgacagaaacagcaaggccacgggcccctgcttcttttccattccgcaccggatcttcagaccttacgcctcgccggacgatttgtcagttccggcactatgctggtttgaaccagcagataaaaatagcaaagaggcggcaggaaaagagattactccgcctgcggacgcccaacctcaaagccaggcacccttttttgttttacacaaaa

SEQ ID NO: 107 - DNA, 1755 bp, Brucella suis
gtgaagcgtatcaggaaccggaaacaggcgctgctgattgcggcattgccgcttcttgtcttttgtggcccgccacaagccatggcgcaggatgtcagtgccgcgcgtccggcagtgcttccccttcccggcgaagccgaaggagccagtatcggctccatttccgttcgcctgcgccaatccagcggcaatgcagaacgcgacgccgccgcgcaggaagccgcacgccgcatcaccaaatccctcgacggatcggaattcagcgccatcgaagccggcgaaattattgccgaactcattcgtcaggaagcaatctccgacgccagattttatctgctcccatctgcctcgcaaaccggcgtcaaactggtttttgaaatcgacgccacttcaggcaccaagacgaagcaagagccaaccggcctttttctcggcgacacatcctcctttccagtcctctatgaaagcaaccgcgcagtactgagaaccattatcggcgccggattgggcggctattccgatgtgaatgcatggttcggaaggccggatctgttcaatggcaggaatccgcttgctggcaaattgccgggccgctcaaacacatggaacgaaggctatctggagcttggcgtcggcggcgccgcgcaattgggcgaaagcaacctctatgggttcggcgctgtcaccggcatgttttcctggaccctcggacaggatatttttcgcgatgacgatcgcggcttcctcgacttcgagaaagcctatgccggtgtgctctatgtatcgccgaagaaccggaaaaaccatttcaaattctcggccgggcggcaggcttatacactcaatgacgggtttctggtgaatgccgttcgtggctccaccagcgccggcgaacgcggcgggctttatctcggcccccggctcaccaacgacttttcggttctggcgcaaggtgcttatgatcgctggaactatagtgccttcttcatcgatcccaacgaagttgaatcgctggaatccgacaccacttttctcggcgcaaatctgcgttacaacttcaccgacaagttttctgccgacactacattaataacgatccccaactcgaaaacacaattttccaccccctctggcagattaatcccgcgtgaagggctgaacacggtttcaggccatttgaaatggaccagtgctttcgagatgcccggcctgtggctcgaaggcgaactggcgctccagtttcaccccggtgaggatgtgtcggcctgggcctattacggaacagtcggctatctcatgaaagagattggctggacgccaagtctttcctatcgctacgcctatttttcgggcgacgatccgaactcatcgcgttatgagcggtttgatcccctgctctcgaccgggcttggcatctggttgcagggtgtttcgtttggaaagatcaccaccaattccaatcttgagacgcaccgtgtccagttcaatgtcgcacccgacgagcggctgaacctgacattcgactactatctgctgcgtaccccccaattgaacaatctcggttcaaacccggcgctttcgacgctatcgtcgcacgatcttgggcaggaactttcactttcagcacgctggagcgcgaccaaaagcctttacctgcaagcacttgtttcacatgccattcccggcacggcgctacgcgatatcggtgccgaccggaactggacgacttttcaggtcagcctctacgggggcttt

SEQ ID NO: 108 - DNA, 837 bp, Brucella suis
atgtcgaaagcacgcacttcacccgttcgtaccgcactcgttcacgcgaccctgatcttctatatgttgatcgccgtgtttccggtggcgctgacgctgatcaattccttcaagagccgcaatgcgatcttccgtgagccgttgcggtttcctacgccggaaagctttagcctcatcggttatgaaaccgttctgaagcaaggttcgttcctgacctatttccagaacagcgcaattgtcacggttgtcagtatttttctcgtcatcctgtttggcgcgatggcggccttcgcgctcgccgaatatcgtttccgcggcaatatgctgaccgggctctatctggcaatcggcattatgatcccgatccggcttggcacggtcgcactgttgcagggaatggttgcagcagggctggtcaatacgctgacggcactcatacttgtctatacggcgcaagggttgccgcttgcgatcttcattctctccgagttcatgcgaaccgtttcggatgacttgaaaaatgccgggcgaatagatggcatgtcggaatatgcgatcttcttcaagctggtgttgccgctgatccgtcccgccatggctaccgtcgccgtctttaccatgatcccgatctggaatgatctctggttcccgctcatcctcgcgcccagcgaggccaccaagaccgttacgcttggcgcacagcttttcatcggtcagttcgtcaccaactggaatgcagtcctcgcggccctgtcattggcgatgttcccgatcctcattctctatgttctcttctcgcggcagttgatccgtggcattacggctggagcggtaaaa

SEQ ID NO: 109 - DNA, 126 bp, Brucella suis
ttgcttggctttctgtttgatggcgacgccgaagttcatttctttcgcaacgacttccacggcgttggctgcatcatcggctggccgccctttttcgcgtttcccctagaagagcggttcctgttt

SEQ ID NO: 110 - DNA, 726 bp, Brucella suis
atgagcccccacgacagcaacatcattctcgatgtacgcaacctatcggctggctatggtgagctgccggtcattcgtgatcttaatcttcatgtcaaggaagcggaaatcgtagcactggttggcagcaacggcgctggcaagacaacgtttctgcgcgcgctttctcatctgatccattccgagggccaaatcgagttttgtggcgaagacgtttccagatcgacatcggattatgtatttcgacgcggattggtacatatccccgaagggcggcaactcttcgaccgtatgtcggtggaagaaaacttgttgatgggcgcttatcgcagcaaggacaaggcccggaccaaggctgatctggaaaaggtctatgttctatttccccgcctcgccgaaaggcgcaagcagttggctggctccatgtcaggcggagagcagcagatgtgcgccatggctcgggcgctgatggccaatcccgttttacagctggttgatgaaatgagcctcggccttgcgcccgtggttgtcgaccagttgatgacgatcctgcatgacattcgcaagtcagagcaggtaaccgtgcttctggtcgagcaggatattcatcttgctctggacggcgcagaccgtggatatgtgatggaaaccggcaccttcacgctggaaggcaaggcgcaggatcttattcacgatgagaggatgcaaaaagcatatttgggtttg

SEQ ID NO: 111 - DNA, 672 bp, Brucella suis
gtggagcgaccctaccagtcgcgcacgctgcgcacgcgtgatccgcacggcattccgcttgagttctattccagaatggaccgcctgccgccgatccatcaaaaatatgcgctctataagggcgtgaagccgctgcgcatcgaccacttcaactgcttctcgcctaatgtggatgaatccgtcgccttctataacgagctgggtttccgcgtgacggaatataccgaggatgccgaaactggccgcctgtgggccgcctggacgcaccgcaagggcggcgttcacgatatcgccttcaccaatggcaaggggccgcgcctgcaccataccgctttctgggtgccgacaccgctcaacatcatcgacctgctcgacctcatgtccaccaccggctggcttgccaatatcgagcgcggtcccggccgccacggcatttccaatgccttcttcctctatgtgcgcgacccggatggccatcgcatcgaaatctactgctcggattaccagacggtcgatcccgatctggagccgatcaagtgggatctgaaagacccgcagcgccagacgctctggggtgcgcctgcaccgaaatcctggttcgaggaaggcagcttgtttgccagtgtgcagccggtcgattccgtgttgaaggcacagccgatcgtcgcgccg

SEQ ID NO: 112 - DNA, 981 bp, Brucella suis
atgttcagccgattcttccgtaatgaccgacgctctttcgattcgctttctgaacaggaaattctggcgcttgccatatcctccgaggaggatgatgcccgcatctatctggcttatgccgatggcctgcgcgacgagtttccgcaatcggcgaagatttacgagcagatggccgcagaggaacacgaccaccgcgcagcactgattgagcggcataaggcccgctttggcgaccggattccgctgatccgccgcgagcatgtcagtggctattatgatcgcaagccggactggctggtgcgtccgctcggcatcgacaaggtgcgtgaggccgcctccgagatggaggaacaggcttatcgtttctatgtcgaggcggccaagcgcgtcagcgatgcagacacgcgcaaactgctgggtgaccttgcccagcaggaaaaacagcacgaagccaaggctgaatcgctggaacacacgctgacaccggataatgtgcaggatgaggaacgcgccgccgagcgcaagcaattcatcctcacctatatccagcctggccttgcgggcctgatggacggctccgtctcgacactcgcaccgatctttgccgccgcctttgccacgcaggatacatggcagacctttctggttggcctttccgcctcggtcggcgccggtatttcgatgggctttacagaagccgtgcatgacgacggcaagctgtcgggacgcggctcgccgatcaagcgcggcatttcatgcggcgtcatgacgacgctgggcgggcttggtcacagcctgccctatctgatcaaggatttctggactgcgacaagcatcgccgtgatccttgtcttcttcgagctctgggccattgccttcattcagaaccgctatatggaaacgcccttcttccgcgccgcactccaggtcgtttttggcggtgcgctcgtcctggcggcgggcatattgatcggcagcgcc

SEQ ID NO: 113 - DNA, 834 bp, Brucella suis
ttgcagggtaaaactgacgtgatattccaggccctatctttcggaagagacctacggccaaccggtgcgcatttgccgggatggcggagtatctgcgcggttatgggactgacgttcaccggctgtgttttcatccagggatcagccagagcgcaggatggcaacaaggcaactgccgtgctggagctttacaccagccagggctgcaaatcctgcccgcccgccgacaagaatttcgccaaatatgccgacgatcccaatgtgctggcgctttcgttccacgtcaattattgggattacatgggctggcgcgatacgctggccacccaggacaataccgaccggcagaattcctatcgcaacgcttttcatgccaagatgatctatacgccgcaagccatcatcaatggcgcagtggaagcaaacggacgcaatggcgcggccatcaatgcgaagatcgccgcaaacaagctggacgtgcccgttagcatccagcagctcaacgatggacgcctttccatcgatatcggtgcaggcaagaagcccagggatccggttcacgtggttctattctatttccgcgattcggccaccatccccatcaccgagggtgagaatgccggaaaagccgtcacctatcgcaatacggtgagcgatatcgacacgatcggcatgtgggatggaaagccgctcaagatcgagcttccggcctccgagctgaagcgcaaagacgtgacgggctgtgccgtggttctgcaagaaacgcgcagcgacaattcgctgggaccgatgcatggcgccgccatgttcaagcagaagcgtcccgtctccttc

SEQ ID NO: 114 - DNA, 264 bp, Brucella suis
gtgaacgaggctgatgcgcttgatatcgtcaattcggcgatctggaccgtgctgacggcaagcgggccggcggtgcttgccgccatgcttgcgggcattgggattgcgctgtttcaggcgctgacgcaaattcaggaaatgaccctgaccttcgtgccgaaaatcatcgtcattttcgtcgtcctggcgctgacggcaccttttgtcggtgcgcagatcaatgctttcacgctgcttgcctattcccgcatcgaaaagggtttt

SEQ ID NO: 115 - DNA, 909 bp, Brucella suis
ttgcgcaagccgtacagtccatcgacagcggcgcagcccttgcggttttgcaaaaggtcattgccgtttccaacgacaagccggcctgacaggaaggatcgcgccatgtccaccgatattcttcgcaagatcgaagcctataagcgggaagaaatagccgccgccaaggcgcgtcttgcccttgatgaactgaaggcccggacgagggatcagtcagcgccgcgcggttttctgaaggcacttgaggccaagcgcgccgccggtcaatttgcactgatcgcggagataaagaaagcaagcccgtccaaaggcttgatccgccccgatttcgacccgccagcgcttgcaaaggcttatgaagaaggcggtgcggcctgtctttccgtgctgaccgacacgccttcctttcagggcgcgccggaattcctcacggccgcacgccaggcttgcagcctgcccgcattgcgcaaggatttcctgttcgacccctatcaggtctatgaagcgcgtagctggggagcggattgcattctcatcatcatggccagcgtcgatgacgatcttgcaaaagagctggaagacacggctttcgcactgggcatggatgcgctgatcgaagtgcatgacgaagctgaaatggaacgcgccctgaagctttcctcgcgcctgctcggcgtcaacaatcgcaatctgcgcagcttcgaggtcaatctcgccgtttccgagcggcttgcgaaaatggctccgtccgaccgtctgctggttggcgaaagcggcatcttcacgcatgaggactgcctgcggcttgaaaagtccggcatcggcacttttctgataggcgaaagccttatgcgacaacatgacgttgcggcagccacccgcgcgcttttgacgggtgcggaaaagcta

SEQ ID NO: 116 - DNA, 777 bp, Brucella suis
gtggggccgcttgcaaccggcttttggcgtctggcgctggccgtactgccgctgttgctcatggatgggcgtgagcgtcatgagagaaaggatgcagcgcctctcacgcttgcagattgcctgcggctcgcattgcccggctttttcattgccgccgatctcggcgcatggcacctgtcgctccattccacatctgtcgccaatgcgaccctgcttgcaaatatggcgcccattttcgtcaccatcggtgcatggcttctgttccgctcgcgcgtcagttcatccttcattgtcggtcttgtggtggcagttgcaggcgtaatcgtgctcaagggcggccccggcgaattgctacacagccgctcgtccggcgacgccatggcgcttctcgccgccgttttctacgcgggctacatcctctcggtcggccgtttgcgcagccgcttttccaccatgcgcatcatgttgtggagcagcgccatcgcggccattttctcgcttccggtcgcctatttcatggaagggcatatcctgcccgcaacgctttacggctggcttgtcctcgcgggccttgcctggctcacccatgcagcaggccagagcatgattgcctatgcgctcgccttcctgccagcctccttttcctccctcacgctgcttttgcagccggtcgtagcggccttcctcgcctggaccatcctgtcggagccgatcgggctgatgcaggccattggcggtgtcatcgtcatttccggcatattgatcgcccgacgagga

SEQ ID NO: 117 - DNA, 951 bp, Brucella suis
ttgctggcttacatatccgggcggttggtttcgctcctgcttacgacgcttgcggcctccgtcatcgtcttcctgctgatgcaggtgctgccgggcgatccggcggcggtgatcctcggcattaatgcgcagcccgagacgctggccgcattgcacaagcaactcggcctcgatcaaccgctctggtggcgctatcttgaatggataggcggctttctcaaaggcgacttcggcacgagctatacctattccgtgccgatcagcgaattgctcgggccacgcatcatggtcacgctgccgctggcgcttctgtccatggtgctgtcggtggccgttgccattccggttggcgtctatgcggcttccaggcgcggcaagaccggcgacgtgctttccatgggcgtggcgcaggtcggcgttgcgatcccgaatttctggcttggcctgcttttgatcctgttgtttgccttgcagcttggctggtttcccgcttcgggttttgcaggctgggaaggcggtttctggaatggcatccgctcgctgtttttgcccgcattggcgcttgccctgccgcttgccgccattctggcccgcgttacccgatccgccgtcattgaaactctgggtgaggatttcgtgcgcacagcgcgcgccaagggcctgacgcgcaaggcggcgctctggcgccatgccgtgcccaatgcgctgatcccggtcgtcaccattattggcctgcaattctcctttctgctggcgggaaccatcatcatcgaaaatgtcttcaatctgcccggtctcggaaggctggtatttcaggccattgcgcagcgtgatctcgtcaccgtgcaatcgctggtgacgttgcttgccgcttccgtcatcggggtcaatttcattgtcgatctggtctatggctttatcgatccacgcctttcgacgggaggcagccga

SEQ ID NO: 118 - DNA, 1535 bp, Brucella suis
ttgatgtccgaatatcttttggaaatgcgaaacatcggtaaagagttcaacggcgtcaaggcgctggatggcatttacctaaagtgcgcgcgggagaatgtgtcgagctgtgcggtgaaaacggggcgggcaagtccaccctgatgaaagtgccgtcgggtgtttatccccatggcacatggactggtgaaattttctgggaaggcaaagaacttaaagccagtggcattcgcgataccgaagctgcgggtattgtcatcatccatcaggaactgatgatggtgccacacctgtcggtcgcggagaatatcttccttggttgcgagccaaccacaggcggtttcattgattatgaccagatgaatgcacgggcagccgaactgctcgctcggctcaagataaacgatatcaatgtcgcattgccggtttatcattactcaggcggcaagcagcagttgatcgagattgcaaaggcaatcaacaaaaatgccaagcttctgatccttgacgagccaacctcggccctgaccgcttcagaaacccgggttctcatcgacctgatcaaggacttcaaaaagcagggcatggcttgcgtttacatctctcacaaactcgatgaagtggcggaaatctccgacacggtaacggttattcgtgatggcgcgcatatcgcgacacgtccgatgtccgaactgacaacgcccgatatcatcacgatgatggtcgggcgtgagatgaagaacctcttcccgcgtgaaccccatgacattggcgaggtgatgtttgaagcccgcaatataagttgctgggatgtgaccaatcctggccgcaaggtggtcgacgatgtttcgtttgcgctaaggcgcggtgagattttgggcattgcgggccttgtgggtgccgggcgtacagaactcgtatcgagcttgtttggtgtctggccgggcgcctgtcaggggcaggtttttctggaaggcaaggaaatcaaaatccgcacgccgcgcgatgcggtgcgccaaggcatttgcatggtgccggaagatcacaaacgcgatggcatcctgccgatcatgccagttggccacaatatgacgatctccgtgcttgatcgcttttcgctacgaggcctcatcgataaagacgcggaactcgtggcgatccagcgtgagattttgagactcaaggtcaagaccgccgatccgatgcttgccattgcctcgctatctggcggcaatcagcagaaggccgtgctttcaaagatgatgctgcctgatccgaaagtgctcattcttgatgagcccacacgcggcgtcgatgtcggcgcgaaatatgaaatttacaagctgatattcgcgctcgccaggcagggcgtatcgattttgatggtttcgtcggaaatgccggaagtccttgggatcagtgatcgtgttctcgtcattggcgagggcaaattgcgcggcgatttcccgaatgaaaatcttacacaggagaaggttcttgccgctgcaattggcaagcctgcaaccaacgcggcc

SEQ ID NO: 119 - DNA, 1524 bp, Brucella suis
ttgtctcctgcgctttcttctgcccacaggaaatccacgatgaacgtcctctatatcgacattgacagcctgcgccgcgatcatctgggctgctacggatatcaccgcaacacgtcaccgactatcgacgctatcgcgcgggaaggcgtgcggtttgagaatatctatgtttcggatgttccctgccatccgtcgcgcacggcgctgtggtcgggcaggcacggttttcgcaccggagtggttggccatggaggcacggcctgcgagccttttcgggagggggcaagtcgcgcctgggcaggcaccttctatgaagaaggctggatgaaagctcttcgcgatctggactattacaccaccaccatatccagcttcggcgagcggcatggctgctggcattggtatgccggtttcaacgaggtcatgaattgcggcaagggcgggatggaaaatgctgatgaaatcgtgccgatggccatagactggattgcgcgcaacaagtcgcgcaaatggttcctgcatgtcaatctgtgggatccgcataccccttatcgtgtgcccgaggaatggggcgatcctttcgccggtgagccgctgcccgcatggatgacagaggaggttctggcgagaagcatcgctggttacggtccgcacagcccacaggaaccgaccggcttttccgcggacaacccacatgcgcattacaccagaatgccagccccgatagattcgatggagaaagttgcggcctggttcaacggttatgatgccggtgttcgctacgcagacgagcacatcggccatctgatcgcggcgctgaaggagcatggtctttacgacaatacgatcattgtgattggagccgatcatggcgaaaatctgggtgagctgaatgtctggggagatcatcagacggcggatgaattcacctgcaatgtgccgcttatcattcgctggccgggccaggctccgggcgtaaatcatgggctgcactaccatttcgactggcctgcaacattgattgacgggcttggcggaaaagtaccgtcggtttgggatggaaagtcattcgcaccggcaatgcgccggaacgagaatggcgggcgcgatttcctcgtgcttagtcagggtgcctgggccgtgcagcgcggtgtgcggttccgtcatcagaatgaagattggctgatgcttcgcacatggcatgacgggctgaaggatttcgggcctgtcaccctgttcaatctcacatccgatccgcatgagcagacggatctatcccaaagccgccccgacattgtcgcacatgccagccgtatgctcgatgaatggtatggcacaatggcccaatgcagcgatcaggatgtcgatccgctcatgacggttctgcgcgagggcggcccttatcatacgctgggtgagcttccggcctatctagaacggctccgcgccacgggacggggagaccatgccgacgctcttgccgcgcgtcatccgcagcgtgagaaggaaaaggcaactttgcga

SEQ ID NO: 120 - DNA, 795 bp, Brucella suis
atggcaggagcgggcatgaaacccaaaatatcctttaataatgtggtgatgcgctatggcggttttcttgctctggatcggctgaaccttgatattgccgacggtgaatttgtgacggtggtcgggccttccggttgcggaaaatcgacggcaatgaatattgcagccggacttttgcagccatcaggcggtgagatacttgtgggcgacaagccggtcacgggccccgggccggagcgcggcgtcatctttcagcaatatgcactgtttccctggcttaccgtgcgtcagaacgttgaattcggccttagcgtcgcgggcatgtcgcgcgtcaagcggcgtgaaatctctgatcactacctgtctctggtcggccttacagatttcgccgatgctttaccaaaagccttgtcaggcggcatgaagcagcgttgcgccatcgcgcgcgcctatgcggcagcacctgaaatcctgttgatggacgaaccgtttggtgcgcttgacgcactgacgcgtgtgcatatgcaggaccagttgctggacgcctggagccgcgagcggcgcacggtgatgttcatcacgcatgatgtggatgaggcggtttatctcgcaaatcgcgttatcgtcatggccgcccgccccggacggctggatcagatcatcccggtcgatctgccatatccgcgcaccgaggccattcgtctttccccggaatttgcagccatccgcaaccgcgtctggcatgctgtctaccatcagcagccccaaacagaccagcaatcttcacacggtcaa

SEQ ID NO: 121 - DNA, 381 bp, Brucella suis
atggcgctggaaccggctgctgctgccgccgccgagcgcgccaccagcgacgagatcgtggagctttatgcacttgccgccaaattcgacaatcccgaccacacgccggaatccatcgccaaggtcgatctggaattccatcttgccattgcccgcatgtccggcaatcctttcatgcgttcggtcagcggcttgatcgaggcggcactggccgtttccttccagctttcctcgcccgccgcctcgcaggaggggatcgccgaatgcgcggccaatcacctgcgcatcgcccatgccattgcatcacgcgatccacagaaggcgcgcctggccatggaaagcgtcatcaagcttggcgtggagcgcatccgcgacgtaatc

SEQ ID NO: 122 - DNA, 879 bp, Brucella suis
atgaaactgatggaatccgcgccttttctgctgattgccacgggcacgctgcttggcctcatattgccgctcggcaagatcgccgcacaggcgggaataccgccggaaatgtggacttttctcttttccgcaagtgcgggcgtcatcctttttgccgttctgtgcctgcgtaaaaagaggctcggcttttccggcgggaggcttcgttattatatctgcacagccctgatttcctatgccttgccgaacttcctgatcctgtcggcgatcccgcatcttggcgcgggcttcacgggtatcatgtacacgctgtcgcctgtcatcacgctgcttctttccatgggtttcggcctgcgccgcccgaatgcactgggcatcggcggtattgccatcggtttcgtcggggcggccatggtggcgatgacgcggggggaggtcggcaagccgaccgatctcctgtggattgcagcagcacttctgatccccgttcttttggctatcgggaatatttatcgcacgctgaactggccgaaaaactccgagccgacagagcttgcggcgggcagccacctggcttcggcattcatcctttttatctgcatcactctcttcaccgggcgttttccgctggaaagctttgcgcttgcaccctttgcagcacttgcgcaatccctggctgcggcaggcatgttcgcgctgttcttccggctgcaggcggtgggtgggccggtttatctcagccagatcggctatgtcgcggcggcgctcggccttgtttcaggcacattgtttctgggcgagcattatccgccgctgacctggattggcgcggctgtgatcgcggctggcgtggccatgaccacgcgcgcgcagaaagga

SEQ ID NO: 123 - DNA, 471 bp, Brucella suis
gtgaaggcgatttttcaaacggcgcggctggctcttgctggtggctttctgctggcagcaatggctggcgccggtgcggcagaccgcattgcttttgtcgtgccttcggcgaccgtctatcccggacaaatcgtgagcgataccgcgcttctggaaaagcagttcttcatcaatgcgcctgcggcgacccaatatgtgctgtcgctggatcaggcggtgggcaaggtggcgcgcaagacgctgcttcccggcaagccgatcctcgtttcggcactgggcgagccgtctctggtcacgcgcggcattcctgcaccactggtctttaccgccggcacgctgactataactgcgatgggtacaccgctcgaatccggtgtggctggcgatttcatcaaggttcgtaacgtcgatagcggcatcatcgtgtccggcacagtgctcgcaaatggtcgtattcaggtggggatgcag

SEQ ID NO: 124 - DNA, 420 bp, Brucella suis
atgccaattctttacacgacccagtccacggcaaccggtggccgcacgggcagcgccaagaccgccgatgggcgcctcagcgtcgttctcgacacgccgaaggaacttggcgggcagggcggtgaaggcaccaatcccgaacagctttttgcttccggctatgctgcctgctttctcggggcgctgaaattcgcggcggcgaaagaaaagatttcgatccccgctgaaagcaccgtgaccgcgacagttggcatcggcccgcgcgaggatggcaccggtttcggccttgacgtggcgctgtcaattgctcttcctggcatcgacaaggcaaaggcggaggagctggtgcaggccgcccacatcgtttgcccctattcgcacgcgacgcggggcaatctcgacgttcgcctgagcgtagcc

SEQ ID NO: 125 - DNA, 573 bp, Brucella suis
atgtccgtcgtcctgccgccggtcagctttcaaactatttcagggcttggcccccaactattctcggtgcttgcgctgtggggctgttcggtgcacaaatcgggcatgaaaacaattgttcgtccccgtcgttccgtcctgtttgttcctgccgcaaatacgcgggcattggaaaaatcgctgacgctttccgccgattgcgtgatctatgatctggaggattcggttgcgccggaggcgaaggctgcggcccgcgaggcacttgcagcgcatctttcggcccatccgaaagttgctttcgagcggatcgtgcgggtcaatgcggcagacaccccatggggcagggacgatgttgcggctgtggcgaaaatggctgtaaacgccgtccttttgccgaaggtggagcgcccgcaggatgtgatcgaggccgccaatcatctcgaccggcaggatgccgatcccgccatgggcgtatgggccatgatcgaaacaccgcgcggcattttgaatgtcgatgagattgctgttctggggcatcgctcggcggtgcggcttgcctgttttgtggtaggg

SEQ ID NO: 126 - DNA, 351 bp, Brucella suis
atggctgtccgccgcatcgttgccaatatttccgccagtgatcctgcggcagcaaggcgcttctatggcgacttgctgggccttgatgtggcgatggatttcggttggatcgtcacctatgcaggcgatgccgaggcgcgggtacaggtaagcgtcatgagcgagggcggcgcgggaacgccggtgcccgatctctccattgaggtggatgatgtcgatgctgtgctggagcgcgccatcgcgcttggattttccatcgaatatggtccggctgatgaggaatggggcgtgcgtcgtttctacacgcgcgactcttatgggcgcctcgtgaatattctgacccacaagcat

SEQ ID NO: 127 - DNA, 1047 bp, Brucella suis
atgacgcaaaacgaacgtataggcggagattttcgttggggcatatggggcacggacatgattgcctctagctttgccgccgacattgccgcaagcgccggaatgcgcgttgttgcggtttgttctcacacgatgcaaacggcacaagctttttgcaggcatatcggcattaacaaggctttcggcaatccgcatgcgtttctggccgatcccgaaatcgatgccgtctatatcgcctcgcccaatatgctgcatgtggagcaagccctggatgccataatggcggggaaagcctgcctcatcgaaaagccgctatcgcttgatccagaaggcgcgcgccagatcgaaacggcaagcaaggcgcgaaacatctttgccatggaggcaatgtggacccgtttccttcccgcggtgcaagcggtgaagcacgctatcgatgcaagccgcatcggcacggtcacgcatattgaagccgatctttcctatagccgcgcctatgacccggaaagccgctttttcagcccggcgcttggtggcggagccgcttttgatcttggcgtttatcccctttcgctggcgctttattttctgggcctgccagaaaaggttgatggacattgccagcgcgcggcaagcggcgtcgatttgcgcagtgaattcaacctcggctggcccagcgcaaccgcctggatttcctgcggcttcgacagggacggggaaaaccggatgttgatcgagggaactgacggcgcaatcctgatccacccgcccttcctgaaagcgcaaaggctgacattcttttcgcgttcggccctgcactcgcccttcggcccgaaaaacagcaagggccgcatcggtaacatcatcaatcgcctgccgctgcccggccgcacaatcgaaactcatgcctttgcaggcaacgggcttcaatttcaggcacaagccgtgcgcgatgccatccggcgcggggaaatttccacccccatcatgccgctggcacagagcgcggcggtcgccgatatcatcagcagggttttggtatcgggt

SEQ ID NO: 128 - DNA, 744 bp, Brucella suis
atgctgattgtcgagtcgcatcgcggcaattgcaatgcctataatgcgggctggcttacggccatgacctgttttccgaacttcaaatatatcctcgtcatcgatgatgatgaactggccgatccgatgtggatcgagaacatggtttccgcggccgggcggttcgatgcaagcctcgtcggaggcccgcaatatccggtctttgaaaagccgaattcgcaacaatgggccaaacacccggtttttatgccgccttatacaaagacaggcgctgttccgatcatctattcgtcgggaaacctgctgatcgcgcgtcctgtgctggaggccatcggctatccgttcctggacctgatgttcaattttaccggcggcggcgattccgatttcgtgggccgctccaaggcgaaaggtttccggattgcctggtgcgcggaaggtatcgtgcgtgaaaccattccggcgcgccgtctggaaggcgactggatcagggcgcggggcttgcgcaacggcgttctttccactcttactgaacagcgtcggcgcaaggatgagccttgcgggcagatacgtgtgttcctgaagagccttgcgctattggcttattcgccgatcaaggccttgcggcgcggtcttgcggcaggctttgcgccggctggaatgtattttatctatgttggattgggacgggttttggcacattttggatatctgaatgagcaatatcgtaacccggacaagaac

SEQ ID NO: 129 - DNA, 741 bp, Brucella suis
atggcaacaccgttccgcaattgcaaaaccccgccgaagaatccatcgaaatcggcctttccaccgaaaaccatcgccatcacatcgaatttcggcggcacggggctcacgattttcggggcgctcgacaatgccgatccgatgatccagcggcaggggcgctacgatatcattgtcgtgctgcaaggccctgcgcgcgatattgtggtgcgcaagaaagaacgctatctcggcgtctgggtgaatgccgcttccgaaacctttctcggcgtcccgctctcctattcgctggcctccacccgcaatttgcaggacattgcagatgagaaaacctatcgccagctttcggtcggcgtgcataatttctatctggtgccggacgaccccgacagcccgtccgataatattgtcaaattcggcaacgagctgcgcgaaataaaaatccggcaggggctatatagtcagcgcattggcggtgtggaattcatctcacgcacgcttttccgcgccacgctcaacctgcccgccaatgtgcccgtgggccgccacaaggcgcgtgcgcttttgttccgcaatggtgttttcctgcgggaagccaatgcaagtctggaaatcgtcaaggccggtcttgaacaaagcgtctataacgccgcccaccagaacagccttctctacggcctttttgctgttttcctcgccatcttcaccggttggttcgggcgcattctgttccgcaaggat

SEQ ID NO: 130 - DNA, 105 bp, Brucella suis
ttgccgattgtgctggcaagcacgatccggcttttgtttgctcaattcatccgattgaaacagatcaaagcatctcgcatgaatatggaaaaacgcgacgcattt

SEQ ID NO: 131 - DNA, 1044 bp, Brucella suis
atgacaggagaggacatgcaagcgattatcgacgccgaacagagtttcaagtttccggttctcgtcggcgatatcggcggcaccaatgcccgtttttctatcctcgtggattcaaacgcagagccgaaggagtttcccgtgctccagacggcggattatgccacaatagacgaagcgatccagcacgccattctggatcagacggccatccagccgcgctctgtcattctggccgtggcaggcccggtggacggcgacgagatcgacctcaccaattgcgactgggtcgtgcgtcctaaaaagatgatcgctgatctgggctttgaagacgtgaccgtcctcaatgatttcgaggcgcaggcccttgccgtggtttcgctggaaggccaccatatggaacagatcggcggcaaaccggaggaggctgttgccacccgcgtcgtgctcggccccggcacgggccttggcgtggcaggtctggttcgcacacgtcatgcatgggttccggttcccggtgaaggcggtcatatcgatatcggtccacgcaccgaacgcgactaccagattttcccgcatatcgaacgcatcgaagggcgtgtcaccggcgagcaaattcttagcgggcggggcctgcgcaacctctatctgggcatctgcgccgccgacaagatcacgcccacccttgagacgccagtagacattacatccgccggactggacggcagcaatccacaagccgcagaaacgcttgacctcttcgccacctatctggggcggcttgcgggcgaccttgcgctcattttcatggcgcatggcggcgtttatctttcgggtggcatcccggtgcgcatcctttccgccctcaaggccggttcgttccgcgcagccttcgaggacaaggccccgcacaaggccatcatgcgcgacataccggtccgcgttatcacatatcaactggcggccttaaccgggctttccgctttcgcccgcaccccytcgcgctttgaagtttcgaccgagggccgccgctggcgcatgcgccgc

SEQ ID NO: 132 - DNA, 192 bp, Brucella suis
atggcgcggctcttagcacagaataacccgctcgtcatcccgcatgaaatctggcgtgtcgtgcaagattgcggggatgggcgcaaaagaaaaaagccgggcacgaagcccggctttgaagttctgcctgatcttctggattatttgcacttctggatcgcggccagtgccgcggaaatgcccttcagggaa

SEQ ID NO: 133 - DNA, 453 bp, Brucella suis
ttgccaattgagacgatcttgcgttccgtccttgcgcgcaatggtgccatcgaacttttttcgcgccagcgcaacatcgccggtaatatcgattacgctcaaatccgttgcccggaaaatgccttcagccagaggctcggcatattcgatagccgcactttccgcagcctggcgcagccattcgtcgcgatagctggcaagcgtggggaagcctgcatcccatttatccgggtcggggttgttatgggcatgaatgccgaggaagcgcgcctcatcgaaatcatccgccaccatggaccagtcctgcgcgatgaatgcctcgatatcgcggcgcacgaacatttcccatatttgctcgcggtccttgtcgcctgcgaacggattaaccgctcccttcattctatcctcctgattatcttaattttccgcgtgcggcgacgggcaggatatcga

SEQ ID NO: 134 - DNA, 711 bp, Brucella suis
gtgctcgaaaaccgatatgctcatcttttaaatatcgaaaaagaccgtttctttttcgccctgaaggtggatatcgaagtgatagacgtcgccctcgcgctggccgatcaaggttggcacgcgcatacgatgctctatgcgcgccaataccggatcttcggcattggcggcttcttcgtcggaaaaatacatccgcgtttgcaggccgaggttaatgccgcgcgctacgatccacaaggttacatgtggcgccatgagccgcccgtcccggaagggaacgcggccgggcttgatcgtctcgaagatgaattcgcccgtttccatattgctggggcagcggccccagccacgaaaatgcggatcggccttgccacggctttcgctcgggctgttatagagcccggcagcatcggcctgccagatttccaccagcgcatcgcgcagcggcgcgcccatgccgtcgataacgcgccccttgatagtgatgcgttcgccatgcgccttgtcgttataaagcggacctgtacccagatcttctgcaaaaactccgttaatgccgacgaaattgggcgtaaggccgatatggacgtaaggaccggctgtctgggaagcggtctccttgagataaccgagcggctgtaccatgtcagttgccctccgggcggttttcaaagaaggtcgaacggcgtccgcgcaatacgatatcgaact

SEQ ID NO: 135 - DNA, 1005 bp, Brucella suis
atgcgctcctccggcagatcgaaatcgaaaagatcaacacgcatgacttacctcgtatgggcatttccagcgaaagcgcgaagcaatatcgcgtttggaaacgcaacgcataaagtcggccccggccgaagccggagccgagatttctcgatacaaactattgaatcagatgtcggcggcgatacgcgcggtcacgatcttgtccggatcggaaaccggctcgccacgcttgatctggtcgacattatccatgccttcgatgacctgaccccagacggtgtactggcggtcgagccatggagcatcggcaaagcagatgaagaactgcgaattggccgaattcggattggcggagcgcgccatggaggccgtaccgcgcttgtgcggcgtgttggaaaattcagccttcaggtccgccttgtccgaaccgcccatgccagcgcgcgaaccgtcgaaatgagcgccacccgacttgccgaacttcacgtcgccggtctgcgccatgaaaccgtcgatcacgcgatggaagacaacgccgtcataagcgccttcgcgtgccagttccttgatgcgcgcgacatggcccggtgcgagatccgggtagagctccagcaccacattgccctttgtggtttcgagaacgagcgtattttctgggtctttataagccattgggcctctcctgttgatttgcaccccgaaagttcaaccgtttccggggcgccagatgggtgttacttcgtgtcagcttcgatggtggccttgatgattttgtcgggattcttcacggcaccattctcggcttcgctgcccttcttaatcttgtccacggcatccatgccgctgacaaccttgcccacgaccgtatattgtccattgaggaacgggccgtcatcaaacatgatgaaaaactgtgaattggccgaattcggattctggctgcgcgccatgccaaccgtgccgcgcacgaaaggttccttggaaaattccgccgggagatcgggg

SEQ ID NO: 136 - DNA, 237 bp, Brucella suis
atggcgcaaaccatcggcctttttcagatgccaagactggaggaattggcgatctcaaatctttatcggcagcctgctcaggcgcggcctgcaccccatgtgcctaattggtcagcgcgaacgcatgttgcgcagcgtatcacgatcgaatgcaacctgacgcacacgctggcgcttgctcgcgttctcttcgagaagaagtatgattgccgaggcagccattgcgaccatcatgga

SEQ ID NO: 137 - DNA, 1398 bp, Brucella suis
atgaccgcaagccaagattttgttgtcaaggatattagcctggccgattggggccgcaaggaactcgacatcgcagaaaccgaaatgccgggcctgatggctgcccgcgaggaattcggcaagtcgcagccgctgaagggcgcgcgcatttccggctcgctgcacatgaccattcaaactgccgttctcattgagacgctgaaggttctgggtgccgaagtgcgctgggcttcctgcaacattttctccacgcaggatcacgcggcggcggcgattgcggcaaccggcaccccggttttcgccgtcaagggcgagacgcttgaagaatactggacctataccgaccagatcttccagtggccggatggcgagccgtccaacatgatcctcgatgatggcggcgacgccaccatgtatattctcatcggcgcgcgcgccgaggctggcgaggacgtcctgtccaatccgcagagcgaggaagaagaagttcttttcgcgcagatcaagaagcgcatggccgcaacgccgggcttcttcacgaagcagcgcgccgcaatcaagggcgtgacggaagaaacgaccaccggcgtcaaccgcctctatcagttgcagaagaagggtctcctgcccttccccgccatcaacgtcaatgacagtgtgaccaagtcgaagttcgacaacaagtatggctgcaaggaatcgctggtggatggcattcgtcgcggcaccgacgtcatgatggccggcaaggttgccgtcgtctgcggctatggtgatgtcggcaaaggctctgcccagtcgctcgccggtgccggcgctcgcgtcaaggtcacggaagtcgatccgatctgcgcgcttcaggccgcgatggacggcttcgaggtcgtgacccttgatgatgccgcctccaccgccgatatcgtcgtgacgaccaccggcaacaaggacgtcatcacgatcgaccatatgcgcaagatgaaagacatgtgcatcgtcggcaatatcggccacttcgacaatgaaattcaggtcgcagccctgcgcaacctgaaatggaccaatgtgaagccgcaggtcgatctgatcgagttccccgacggcaagcgcctcatccttctttcggaaggccgcctgctcaatctgggcaatgcgacgggccatccaagctttgtcatgtcggcttccttcaccaatcaggtgttgggccagatcgaacttttcacccgcaccgatgcctataagaacgaagtctatgtgctgccgaagcaccttgatgaaaaggtcgctcgtcttcatctcgacaagcttggcgcaaagctgacggtactttccgaggaacaggctgcctatatcggcgtcactccacagggtccgttcaagtcggaacactacaggtat

SEQ ID NO: 138 - DNA, 2622 bp, Brucella suis
gtgcgccccgagcatgtccgctcacgggccagcaaaaatattcggttcgggacaggctttatgcggcaaggatattcgccctcatacccgctgcgtgacgagcggattacggaagacaggttgcggtttgccaatctcgtccgcaggcaggtctatatccttctggggctggggcttctttcgctcaccgccatggcggttggcgcgcttgccacatggaatgtcgccgatccaagcttcagccatgccaccgacaatcctgtcaccaatgcgctcggctatccgggcgccgtgttctccgatctggccatgcagttcttcggccttgccagcgttccggcgctgctgccgcttgcggtctggtcgcttctcctgatgatccggggttatatcgggcgtatcgcccggcgcagccttgcgtgggtgggggcggcgctgctctttgccgccattgcaagctgcttagccgtgccgcaaagctggccaatgccaatcggccttggcggtgttttcggcgatatgctgcttcggataccgggtttcttccttggcgggttcccgcaaggggctattgcctccgctattgcgctggttctggcttttccagccttgtggttctgtttttttgcaagcggcatcattggtcgcggggcggaagcggtcaatccggccatgctttcggcaaatcgttctgccgatgatgaattcgcggatgaggatgcagacaatgaagccggtggcggtttccacttcatcggtgcgctgacccatctggtgctgatgacgacagccacgatcaggcgcatgaccggacttggccgccgccgctcgcgcgaggatgatttcgacgacatgcgcatggtgcgccgcagtgccgaaacccgcaatgcgccgcgccgcgaacccggtttcggggcgcctgccgccgatgacgaaccgcctttcgatatggacgatatggatgatggcgcaccgcttgccggtcaggaatggcatgatgcgccgccgccccgcgcccgcaaggcgcgtgtggaacaggccgcgccgtcgccgaagcccggcccgcgcgcccagcgcgaggcgcagccttccttccttaaggataatggtatctttgaaatgccgtcgctgcatttcctggccgaaccgaagctcgtgcagcgcgaccccgcgctttccaaggatgcgctggagcagaatgcccgtctgcttgagggcgtgctggaagatttcggtgtgcgtggcgagatcatcaatgtcaaacccggccccgtggtcacgctttatgagcttgaaccggcaccgggcatcaagtcctcgcgcgtcatcggccttgccgacgatattgcgcgctcgatgagcgcgattgccgcgcgtgtggctgtcattccggggcgcaacgcgatcggtatcgaactgccgaaccccaagcgcgaaatggtctatctgcgtgagatgctggcgagccgcgactttgagcaatcgaaggcgaagcttgcgctggcactcggcaagaccatcaatggcgagccggtcattgccgatattgccaagatgccgcacgttctggttgcgggcaccaccggctcgggcaagtcggttgcgatcaacaccatgatcctgtcgttgctctatcgcatgacgccgcaggaatgccgcctcatcatgatcgaccccaagatgctggagctttccgtctatgacggcattccgcatctgctgacgccggttgtgacggacccgaagaaggccgttgtcgcgctcaaatggaccgtgcgcgaaatggaagaccgctatcgcaagatgtcgaaggtcggcgtgcgcaatatagacggcttcaaccagcgtgtcggtctggcgcagaaaaagggcgagccgattgcgcgcacggtacagaccggcttcgaccgcaataccggcgaggcgatctacgagacggaagaactcgatctcgaacccatgccctatatcgtcgtgattatcgacgaaatggccgacctcatgatggtcgcaggcaaggatattgaaggcgcggtgcagcgtctggcgcagatggcgcgtgcggcaggcattcacgtcatcatggcgacgcagcgcccgtcggtcgatgtcatcaccggcacgatcaaggccaatttcccgacccgcatttccttccaggtgacgtcgaagatcgacagccgaaccattctgggcgaacagggtgccgaacagcttcttggccagggcgacatgctgttcatggcgggcggcgggcgcatccagcgcgtgcatgggccatttgtcggcgacgacgaggtggagcgcatcgtgcagcatctcaagcttcagggcgtgccggaatatctcgatgccatcaccgaggatgaagacgatgacgagggcggtagcggcccggctggaaccggcaatctggaggattccgacgatccgtatgatcaggccgtggccgtcgtgctgcgcgacaagaaggcgtccacatcctatattcagcgccgtctcggcatcggctataatcgcgctgcctcaatcatcgaacgcatggaagatgagggcattgtcggcccggccaaccatgcgggcaagcgcgaaatactggtgcccacgggcgatgatgatttc

SEQ ID NO: 139 - DNA, 354 bp, Brucella suis
atgaacttcaagacccgtatggctatgcttgccgttgcggcaaccggccttctcgccgccaccgcaatcgaggcttcagcagagcaatgcggcggtgcctcctggtacgcccttacttcccgcacggcttcaggcgaacgcatgaacccggccggactgaccgcagcccaccgcaccctgccgcttggcagcaaggtcaaggtcaccaatcagaaaaacggcaaatcgctcgtcgtgcgcatcaatgatcgcggtccgttcatcaaggggcgcgtactcgatctttccaagggcgctgcaagccgtctgggcttcatcggcgccgggcacaccaaagtctgcttcaagccactt

SEQ ID NO: 140 - DNA, 1077 bp, Brucella suis
atgcgcgttcttggaatagaaacaagttgcgacgaaacagccgccgcaatcgtcgagcgtgacgatatgggcgaagggcggattttgtccaatgtggttctgagccagattgccgagcacgagccctatggcggcgtggtgccggagatcgccgcccgcgcccatgtcgaggcattggacaggctggtggaccgtgccctgaacgacgcgggcctgaaactgtacgaggtggatgcagtggccgctacggctggccccggcctcatcggcgggctgatcgtcgggctgatgacggcaaaggcgctcgccatggctgcacaaaagcccttctatgccgtcaaccatctggaaggccacgcgctgacggcgcggctgaccgacggcctgccatttccctatctgctgcttctcgtttccggtggccacacgcagatggtgcttgtgcgcggcatcggcgattatgagcgcctcggcaccaccatcgacgatgcgctgggcgaagccttcgacaagaccgcaaagcttctcggcctgccttatcccggtggcccggctgtcgagcgcatggcgctacagggcgaccagaagcgttttgccctgccgcgcccgctgaaaggcgaagcacggctggatttttccttctccggccttaaaaccgccgtccgccagaccgcaaccgaattggtgcctctcaccgatcaggacgtcaccgatatatgcgcctcgtttcaggctgctgtcgccgatacgctttccgaccgtgtgggccgctcgctggagcgtttcaagacggaattcccggattgcgccacgccctccctggtggttgcaggcggggttgcagccaacaagaccctgcgtgccgcactggaaaacctctgcacccggcatggctttgcctttatcgctccgccgcttaatctttgcaccgacaatgcagcgatgatcgcatgggcgggggctgagcgcgccgcgacccaagcgcctgattcgcttgacatcgccccgcgctcgcgctggccgctcgacgaaaaatccgcgccggtcttcggcacgggacggcggggagcaaaggca

SEQ ID NO: 141 - DNA, 867 bp, Brucella suis
atgcagactgtgctcagcattgctgtcgatgcccttgcctatggcatggtgcttttcgtcatctccatcggcctttccgtcaccatgggcctgatgcgcgtcgtcaatctggcacatggcgcctttgcgatgatcggtggctatttcgcatcctatgccgcacaacagcttggcctcaattatggctttgcgatcatcattgcggtcgtcggcacgatgattatcgccgtgccggtggaaaggctgctttaccgccgtatctatggtgcaccggaattgacacaggtattgatgaccattggcgtcacgttctgcatcatcggcatcaccaattatattttcggcccgacgctgaagaccattcccctgccggaaatgctgcgcggctcggtcgatctcggctttcacgctattgcaaagcaccgcattttcgctatcggctgcggcctcgcggtggcagtcggcctatggtacgccgtcgagcgcaccgctttcggcgtgaaactgcgcgctgccgtcgataatgccaatatggcggcagccctcggcgtgcgtaccgagatcgtctatgcggtgagctttgcgctcgccatcgggcttgcggccttcggcggcgtggtcggcgcggagcttttgcccgtcgaaccctattatgcgctgcgctacatggtgactttccttgtcgtggtttcggtcggcggggccggttctatccccggcgcactcgtcgcctgcctgttgctcggcgcaatcgacacgaccgggcgttacctcgcacccgaatatggcgaattcttcttttatctggcggtgatcgtgatcgttaccctgttcccgcgcggccttgcaggaaggctggcccgcaaa

SEQ ID NO: 142 - DNA, 1041 bp, Brucella suis
atgctactgccacccatcaaagaaatgcgggcccgccatggcgcgcacgcggtttgccgcatctgtgccgagcgcggctgcatccgagacaaccccttcggccgtcacttcatgggcctgccggccatccggcgtcaggatcatcccccggaaggacaaccggtcgccctcgaccgttgcaagaccggcaataggcgtacggcaggaaccgtcgagagcgccgagaaaggcccgctcgcaggcaagcgcaatttgcgtttcgcgatgggcgagcggcgcaagaagcacatcgatccgctcgtcgccaatgcggctttcaatgccgattgcgccctgccccggcgcaggcgggaaaacggatggatcgagaagatcggtgatgacatcggcaaggccgagccggcgcaaaccggcacaggcaaggaaagtgccatccacttcgcccgcatcgagcttgcgcaggcgggtatccacattgccgcgatacatcaccacctcgatatccgggcgaagacggcgaataagcgcctgacggcgcagcgaggacgagccaacggtggcgccctgcggcaaatccatgaaacgccttgcgctgcgcccgatgaaagcgtcgcgcgggtcctcacgctccagaaaaacggaaaggtgaagcccctctggcaagacggtgggcatatccttggtcgagtggacggcaatatcgattcgcccatccttcagcgcctgctcgatttcctccgtaaacagccccttgccgccaacttccgaaagcgggcggtcctgaatgcggtcgcctgcggtggacatgggcaggatttcaatcgcatcttccggcaaaccatgagcctcctgcaaaaggcggcgcgtgaggtaagcctgtgccagagccagcttgctgcccctggtgccgatcttcaacgtgccattcttgaaggatgctgtttgcatattgcctcgtccatggtaccgggtttcatcattcgttttcatgcatgtccaaaggcaaaaccggtttccacttttgctgaacatgcgccaata

SEQ ID NO: 143 - DNA, 363 bp, Brucella suis
atggcaaattattccgttacttccggactgttggcagttaccacccgccacggcaccaagcgaagagagagaaagtacatgaaaaagctcatgagcgaagccgacgcacaagggcttgccattgaagcactggcctggctcgcgcaggacaaggacctgctgccgcgctttctggcgctgaccggcatcgaggcttcctctatccggcaggcggcacgggagcccggttttctggctggcgtgctacaatttttcctgggacatgaaccgacgcttatccgcttctgcgaggagacgggccatgaccccgccctgtttgaaaaagcggttgcgcttttgcctggcggcttcaaccagcatctt

SEQ ID NO: 144 - DNA, 795 bp, Brucella suis
atgaacaagatgtccaggatcataaccattgcgaaccagaagggtggggtgggcaagaccactaccgccattaatctggcgaccgcgcttgctgccatcggcgagacggtgctgattgtcgatcttgatccgcagggcaatgccagcacgggtctcggcattgaccggcataaccgccctttgtcctcctatgatgtgctgacgcaggcttcgtccgtaccggaagctgcgatgcagacggatgtgcccaacctctttattgtgccatcaacgctcgatcttctgggcattgaaatggaaatcgcccaatcggcagatcgtacccggcgtttgcgcgatgcattgcgtttcgattctggtgtttcggagcgatttacttacgttctggtcgattgcccgccgtcccttaatctgctgacattgaatgcaatggccgctgccgactccgttctggtgccgctgcaatgcgaatttttcgcgcttgaggggttgagccagctcttgcaaactgtcgatcaggtgcgttccaccatcaatcctgaactgtccatacaggggattgtgttgacaatgtttgacagccgcaacaatctggcaacgcaagtggtagatgatgtgcgtgctttcatgggcgaaaaggtctatcgcacggttattccgcgcaatgttcgtgtttcggaagcaccgtcccatggaaaaccagctattctctacgatctgaaatgcgccggtagccaggcctatctgcaactcgcctcggaggtaatccagcgcgagcggcaattacaggctgcc

SEQ ID NO: 145 - DNA, 1017 bp, Brucella suis
gtggccaccggcaaggtttatgaacagcaggatgccttccagcgctggtatcccgcttcgctcaccaagatgatgacggcctatgtcacattccgcgcgcttcagtccggccagatgacgctggaatccccggtgcgcatgacggtcaacgcctccaaggaaccgccgagcaagatggggtacaagcccggctcgatcatgacgcttgatacggcgctgaacatcatgctggtcaagtctgccaatgatgtttcggtggccgttgccgaagccgtgggcggaacggaagctgccttcgtgcagcggatgaatgccgaggcgcggcgtcttggaatgttcggctcgcatttcgccaatccgaatggcctgcatgatccgaacaattatacgacagcgcgtgatcttgcggttcttgccgtgcagttgcgccgcgaatttccgcaatatgcgcattatttcgcgacagaagcgatcgatccgggtgcgggcaagaaagttgaggccaattacaacatcctgcttggccgctatgacggtgcggatggcatgaagacgggcttcgtctgcgcatcaggcttcaaccttgcaggttcggccacccgcaacggccgcacggtgctggcaatcgtactgggtgcggatcgccaggaaacgcgtgcgatccaggccgcgcaaatgatgaccgatgcgtttcgcgcgcgcagttccggcgggccgacgcttgccacgctgcgcccggcgcagacagccaatctcaatcaggccgttgatatgcgcaaggccatctgtagcgagcaggccatggccgaccgctgggacgggcgcgaggtagaaggccggctgaagatcaattcaccctatattcacgccatggaccatgatccgattgccgtgcaggtgaggctggtttccgaaccgggttcgatcaaccggcccggccagcccggcatttcgcgggttccggtgccaatgccgcgtccgcagcgcgcgccaggcgtaaagacaacggccatcaac

SEQ ID NO: 146 - DNA, 237 bp, Brucella suis
atgaccatcaagcacaacatcatcgccttttccgccctcgccctgattgcaggtttctcgcttacgccagccgctttcgccgaagatgcgatgaaaaccgaccatatgaagccggacgcgatgaagagcgataatatgtcaaaggatgccatgggcaaggaagatgccatgcacaaggattccaagaaaatgaagaagcacgacgccatgaaaggtgacgcaatgaagagcggtcag

SEQ ID NO: 147 - DNA, 813 bp, Brucella suis
atgacagatgtcttgctcctgctgaaagccgcatggaagcgatcgatccgctatggtgcattcaccctggcaggactggcaatcgcgagcggagtggcgagagcagatgatctctatagaaccgaatatgatatctcgattttcgggctttccattgcgcgcgcagctatcgaaaccgtcgtcagtgggccgaactataacctgaacggccgtttcgtgacatcgggccttgcccgcattttcgacaatacggatggcaccgtgcacgtcacaggcagtgcggtaaaaggggctatcgtccccaaaagcttcgatctcgcctataaacatggccgcaaggacaagagcacggcaatccgtttcatgaatggagacgtggttgcagcacaaaaccagccacccgtgaaaaaagacgatccctgggtggaaaccatgccaggcgatctcctcaatgtcagcgatccgctgagcgccctcatgatccccgcaaaggatgcgcgttcggtctgcgaccgctcgctcagtgtttttgacggccagacgcgcgtggaaataaagctggccttcaacggcactgaatcgttcaccacgggtggctttactggcgaatcagtcatctgttcggcgaagttcattccggtttcaggttatcagaagggcaagaaatcgatcgattatctggccaacaagagccggatcaccatctcctttgctgcgctcggcaattccggcatctatgcgccggtggtggcgcgtatcggcacccggatcggaacgctgaaaatcgcagccacccggtttgaaaaagtggaa

SEQ ID NO: 148 - DNA, 969 bp, Brucella suis
atgataagatctgtcgtacggggtatcggttcggcattgcctaaacgggtcatgaagaataccgactttgaaggcattgtcgaaacctcggacgagtggatcgtgcagcgcacgggtatccgtgaacgccacattgcgggcgagggcgaaacgacggtttcgctgggagcggctgctgcgcgcgcggcaatcgagaatgccgggcttcagccgtccgatatcgatctggtccttctggcaacctccacgcccaacaatacctttccggccagtgcagtggcaatccagcgcgagcttggcatcacccgaggcttcgctttcgaccttcaggccgtttgcagcggtttcatctatgccatcaccacggctgatctctatattcgcggcggcatggccaggcgcgttctggtcatcggtgcggaaaccttctcgcatatcctcgactggaccgaccgcaccacctgcgttctctttggtgatggtgcaggcgcgatcgttctcgaagcagccgaaggccatggcctgacttcggatcgcggcattctcgccgccaatctgcgctcggatggcaatcataaggaaaagctctacgtcgatggcggcccgtccaccacgcagacggtgggccatctgcgtatggaaggccgcgaggtcttcaagcacgccgtcggcatgattaccgatgtgatcgaggcttccttcgaggcaacgggcctcacggccgaggatatcgactggttcgtgccgcatcaggccaacaagcgaatcatcgacgcttcggcaaagaagcttcacatcgctgaagagaaggtcgtcatcacggttgaccggcacggaaacacctctgccgcctcggttccactggccctcgccaccgctgttgccgatggtcgcatcaagaagggcgatctcgttcttttggaagccatgggcggcggatttacctggggcgcggttcttgtgcgctgg

SEQ ID NO: 149 - DNA, 900 bp, Brucella suis
atgagtttttttcccgcacgaaccgtcaaaaagctgcaaatatccggtaatttttcgagttatcgccgcagtgcgggttatgatagcaagataagggaaacacaagttacggaagcggcatcgggcgggtattggacaggtgacgatgcagtggcacaaagacggacaggaagcagttcgcaagcagatatatggcgccgcagccatcaggatgcatttgtaccttcgttcctgggcacaccggccagattcgcctggtctgtcatgatgcggatattgcggcgcctccaacctgtttcgatcatccttttcctggcagtcatagcctttctgggtggcttcttggtgttcagtgaaaaagtgacgggaatgcagccgcccgttttgagcgaaccggcggaagcaattgtggttttgaccggcgggcaatcgcgcgtcaaggcagggatcaacctcttgaaggaaaagcgcggcaagcgcctccttatcagcggtgtgcatccatccactaatgaggaggcgctgcaaagggccacccatgcggacaagagcctgtttgcctgctgtatcgacctcgaccgtacggcgctcaatacggtgggcaatgcggcggaaagcgagcgctggataagggcaaacgattaccaccgcgtcatcgtcgtcaccaataattaccatataccgcgcagcattctggaaatgtcgtcgcgcatgaaggatgtggagttcattccctatccggtggtgaatggggaaaagcgcgagcagagctgggttgccgaaggtgatacgctgcgtgtgctcttcaccgaatatgtcaaatatctgggcgccgttgttcggcttggcagcgcctcgttttatggcaatggcgcttctcatggcggcgatatgcgcgaa

SEQ ID NO: 150 - DNA, 384 bp, Brucella suis
ttgtgtaacgtcctgtcacgtaaggatattatctgttcaaacgctaaccaggcaggttcggtcatgggaggtaaaacggtcacgcgggctgatctggcagaggctgtttaccgtaaggtaggcctttccagaacggaatcggcggctttggtcgagatgatactcgatgaagtctgtgacgctatcgtcaatggcgaaacggtgaaattgtcgtcttttgcgacatttcaggttcgcgacaagaacgagcgtatcggtcgtaaccccaagacgggtgaggaagtgccgatcctcccgcggcgtgtgatgacgttcaaggcgtctaatgttctcaagcagcgcattttgcaggaacatcagaagcgtcagggtaaaacctcgaag

SEQ ID NO: 151 - DNA, 1398 bp, Brucella suis
gtggagataggtctaacccaaattgcccgaaagtggaatcatctcttggcgggtgattccacatggcatggtgttgatcgtttgtcaggccttggcaatacgctttttccagtagcggatctggcggcgcacttcctgcggggcggtgccgccgaaggattggcggcttttgaccgatttttccaccgtcagatagccgaagacctctgctgtgatggcggggttgatcgactgcaattcctcaagcgagagctttgccagatcgaccttgcggctttcggcaagcgccacggcgcggcccgttacgtgatgggcttcgcggaagggaaggcccagtgtgcgcaccagccagtcggcaaggtcggtcgcggtggaatagccggaacctgcggccttcttcatggctgcgacattgacggtgagatcgcgcaccattccagccatcgcggcaatcgccagttccagattttcggcagcgtcgaagacctgttccttgtcttcctgcatgtccttggaataggcgagcggcaggcccttcatgatggtgagaagtgcgaccagcgaaccattgatgcggcccgtcttggcgcgtaccagttctgcggcgtccgggttcttcttctgcggcatgatcgaggagccggtggagaaggcgtcggaaaggcgcacgaaattgaactgcggcgtggaccagatgacgatctcttcggcaaggcgcgaaagatgcccggcgcagattgcggcaagcgacaggaattccagcgcataatcgcggtcggaaacgctgtcgagcgaattgcgggtcggctcgcggaagcccagcgcctttgccgtcatgtgacggtcgataggaaagccggtgcctgcaagggcggctgcacccaaaggcgattcgtccatgcgctcgatggcgtcgcgcacgcgcgaaagatcgcggccgaacatttccacataggccatgcagtgatggccgaaggtgacgggctgcgcggtctggagatgggtgaaacccggcatgactgttgccgcgtgttcttccgcgcgctccagaaacgcttcgatcaggtttttgagggcggctgcggttttttccagttcctgcttcacccagagacggaaatcgacggccacctggtcattgcgcgaacgggcggtgtggaggcgcccggcggcagggccgatcaattccgccagccgcgcctcgatgttcatgtgaatgtcttcgagcttgcgcgagaaggtgaatttgccctcttctatttccttgcggatggttttcaagccattttcaatttgtttgtggtcttctgccgcaatgatgcccgtctttgcgagcatggctgcatgggcgagcgagccctgaatatcctgcgcatagagcttgcggtcgaaaccgat

SEQ ID NO: 152 - DNA, 378 bp, Brucella suis
atgaaacgcttccgcatcgttgcccccctcgcactcatgtcgcttgcgctggctgcttgcgaaacaacaggcccgggcagcggcaatgccccgatcatagcccatacccctgccggcatagaaggaagctgggtcgatccgaacggcatcgcttcctcattcaatggcggcatctttgaaacccgcaccaccgacaccaacgaaaagctggcggagggcaactatctctacctgtcgccgcaactcgttgaaatcaacatgcggtccatcgtgcgcggcaccacctcgaaggtaaattgcgcactggtttcgccgacgcaacttaactgcacctcctcggccggttcgcgcttctcgctcacccgccgcaacgccggc

SEQ ID NO: 153 - DNA, 990 bp, Brucella suis
atgtcggctctacctgatcatattgaagtaatttctaaaacatctgatctcaggtttgaagacatcgatatcgtcgttacacttcctacatttcggcggcccgatcatctcatccgtacccttgatatgctgaaagcgcagaagaccgaaaagcgctttgcggtgatcgtgatcgaaaatgaggctgaaaagagggaaggggccgaggtggccgctccgctgtttgcggatggcacgtaccggggaatgctgattgtcgagtcgcatcgcggcaattgcaatgcctataatgcgggctggcttacggccatgacctgttttccgaacttcaaatatatcctcgtcatcgatgatgatgaactggccgatccgatgtggatcgagaacatggtttccgcggccgggcggttcgatgcaagcctcgtcggaggcccgcaatatccggtctttgaaaagccgaattcgcaacaatgggccaaacacccggtttttatgccgccttatacaaagacaggcgctgttccgatcatctattcgtcgggaaacctgctgatcgcgcgtcctgtgctggaggccatcggctatccgttcctggacctgatgttcaattttaccggcggcggcgattccgatttcgtgggccgctccaaggcgaaaggtttccggattgcctggtgcgcggaaggtatcgtgcgtgaaaccattccggcgcgccgtctggaaggcgactggatcagggcgcggggcttgcgcaacggcgttctttccactcttactgaacagcgtcggcgcaaggatgagccttgcgggcagatacgtgtgttcctgaagagccttgcgctattggcttattcgccgatcaaggccttgcggcgcggtcttgcggcaggctttgcgccggctggaatgtattttatctatgttggattgggacgggttttggcacattttggatatctgaatgagcaatatcgtaacccggacaagaac

SEQ ID NO: 154 - DNA, 360 bp, Brucella suis
atgatcgctgtcattttcgaagcttgggttcctggtgaagcgcaaactgaatatctcgatctggctgcggagctacgcccgctgctggctgacctcgatggcttcatctccattgagcgcttccagagcctgaccacccccggcaaagttctgtcgctgtccttctggcgtgatgaagcggctgtcgcagcatggcgcaaccttcctgagcatcgccgtgtgcaggcagcaggccgcgaccacgtctttgccgattaccgcctgcgcattgcagaagtcgcgcgggactatggaatgcgcgcgagaggcgaagctccgaccgacagccgcaatggccataaggaggacatcgatcatgta

SEQ ID NO: 155 - DNA, 714 bp, Brucella suis
atgaacaaagcgatccttgcagtagcaatggtgctccttctggccggttgcgccaccaagcccgaggaaattggccgcgcgcccgacctgtcgccggtcgcggcccatctcggcatgcagaacaatccgcagttcaacggctatccggcgcggcccggcaaggcgtcctattcgctctgggatcagcgttcgaccaatttcttcaaggacccgcgcgcggcgacgccgggcgatgtgctgaccgtcatcatttccatcaatgatcgcgccaacctcgataacaagacggaccgcgagcgcgtgtcgaagggcatttacggcggaggcggttcattcgccaccagcagcattacgggcgcggcggctggcggtgatatggatgcttcgatcaacactcattccgacagcaagtcgaagggcaagggcacgattgaacgcagcgaggatatccgcttgcagattgccgccatcgtcaccgacacgctgccgaacggcaacctcattatccgtgggtcacaggaagtgcgtgtgaacaatgaattgcgcgtgctgaacgttgccggtgtcgtgcgtccgcgcgatatttccggcaacaacacgatttcctacgacaagatcgccgaagcgcgcatctcctatggcgggcgcgggcgactgagcgagattcagcagccgccttatggccagcagattctcgaccagttttctcctttc

SEQ ID NO: 156 - DNA, 549 bp, Brucella suis
ttgatggagagtggcatgtccaagcataaaggcccaaccggcgcgatggtcgatcctgaaagcctgccttaccgtccctgtgtcgggctgatggttctcaacaaggcggggctggtctgggccgggcggcgcatcgtcattccgggcgatgaaatggatggcgccacacagctctggcagatgccgcaggggggcatcgataagggcgaggacccggcgcaagccgcgctgcgcgaactttatgaagaaaccggcatgacgtcggtctcgcttctggaggaagcatcggactggatcaattacgatctgccgccgcatctggtggggctggcgctcaagggaaaatatcgcggccagacgcagaaatggttcgcctatcgcttcgagggcgatgaaagtgaaattgcgatcaatccgccacccggtggccacactgctgagttcgattgctgggaatggaagccgatggctgacctgccgaacctgatcgtgccgttcaagcgcaaggtctatgaacaggtggttgcgactttccggcatctggcggcc

SEQ ID NO: 157 - DNA, 528 bp, Brucella suis
atggcatttgtgcatttttcgaatcccgggaaggcgctgcactatgcttcccaaacctttatcgggtgtctcatatcctggttcgccctgcgcggcataggcatcgagcacccgatctggtcgatgatgacggttttcatcgtttccgatccagatattcacaccacgctgtcgcttgcccgcgtgagggcgatcaatacggcagtgggctgcacgatcggcatggtgatgatctggcttgtcggctattcgccggtcatcagcctttttgccgccgcgataacggttggcgtcgtcgtgatgatcgacaactatccgagcaactggcggctggccccggcaacggtagtgatcatcatggatgcgggaagtgctgcccagacaagcgatcaggaattatggcttgccctgtcacggtttcttgaaatcacgcttggatgcgtggttgcgctctcggtcgcgtggatctacagccgcacgatcatgacgatcagaaagcgcctgcggcacggcaaacctgggcagacc

SEQ ID NO: 158 - DNA, 1563 bp, Brucella suis
atggcaaaagcagcgaccccgaaaactaccgccgcggccgaagcaaagccagcggcgaaggcacccgccaaaaaggcggcgcccaagaccaccgcagcagccaagcccgctgctacgaagtctggcgcccccaaggcagcagcagccggtgcgatcggccacattacgcaggtcatcggcgctgtcgtcgacgtcaagttcccggaaggccagctgccgctgatcctgaacgcgcttgaagtggacaatcagggccatcgtctggttctcgaagttgcccagcacctcggcgaagacaccgtgcgcaccatcgccatggacgcgaccgaaggtctcgttcgcggtcaggaagcacgcgacactggcgaaccgatcatggtgccggtcggcgtcgaaacgcttggccgcatcatgaacgtcatcggcgagccggttgacgaagcaggccccatcaagaccaaggcaacccgcgccatccaccagaacgcgccggaatatatcgaacagtcgaccgaagccgaaattctggtcacgggcatcaaggtcgtcgaccttctggcgccttacgccaagggcggcaagatcggcctcttcggcggtgcaggcgtcggcaagaccgttctcatcatggaactcatcaacaacgtcgccaaggcgcacggcggttattccgtgttcgcaggcgtcggtgagcgtacccgtgagggcaacgacctttaccacgaaatgatcgagtcgggcgtgaacaagctcggcggcggcgaaggctccaaggcagccctcgtttacggccagatgaacgaacccccgggtgcccgcgcccgcgttgccctttccggtctgacggttgctgaaaacttccgtgaccagggccaggacgttctgttcttcgtggacaacatcttccgcttcacgcaggcaggttcggaagtgtcggctcttctcggccgtattccttccgctgtgggttatcagccgacgctggcaaccgacatgggcgccatgcaggaacgcatcaccacgacgaccaagggttcgatcacctcggttcaggccatttacgtgcctgccgacgacttgaccgacccggcaccggcaacctcgttcgcccacctggatgcaacgaccgttctgtcgcgctcgatcgctgaaaagggtatctacccggctgttgacccgctcgactccacgtcgcgtatgctcgacccgaaggtcgtcggtgaagaacactatgccgtcgcccgtcaggttcagtcgatcctgcaacgttacaaggcccttcaggacatcatcgcgatcctgggcatggacgaactgtctgaagaagacaagctcaccgttgcccgcgcccgcaagatcgaacgcttcctgtcgcagccgttcttcgttgctgaagtcttcaccggttcgccgggcaagctggtcgatctcgccgacaccatcaagggcttcaagggcctttgcgctggcgactacgatcaccttccggaagctgccttctacatggtcggcagcatcgaggaagcactcgaaaaggccaagaagctggcggctgaagctgcc

SEQ ID NO: 159 - DNA, 906 bp, Brucella suis
atggaatatttcttccagcaggtgatcaacgggcttgcgctcggttcgatctatggcctgatcgccatcggttatacgatggtctatggcattatcggcatgatcaactttgctcatggcgatatcttcatgctcggcgcgttcatggcgctgattgttttcctcattatcacgaccttcaccggaatgctgccggttgcactgatgctgcttttgatgatgatcgtcgccatgcttctgacgggtctgtggagctgggcgatcgagcgcgtggcctatcgaccgctgcgtggttcgttccgccttgcgccgctgatcacggctatcggcatgtcgattgccttgtcgaacttcgttcaggtcacgcagggtccgcgcaacaagccggttccccagcttctgaatggttcctacgagctgttcggcagcggcgtcacgatctcgctcaagcagatcgtggtgatcgtggttacggcaatattgctcacgatcttctggtatgtcgtcaaccgcacctcgctggggcgcgcgcaaagggcttgcgagcaggatcgcaagatggcggcccttcttggcatcaatgtcgaccgtgtcatctcgctcaccttcgtgatgggcgccatgctggcggccgtagccgggacgctttacctctccttctatggcgtgatttcgttcgctgacgggttcatccccggtgtgaaggcgttcacggcggcggttctgggcggcatcggctcgcttccgggggctgttgtcggcgggttgcttatcggtctcatcgaagcgttgtggtcggcctatttctcgatcgactacaaggacgtcgccgcgttctcgatcctggctatcgtgctgatcttcatgccgtccggcattcttggccgacctgaagtcgaaaaggtg

SEQ ID NO: 160 - DNA, 537 bp, Brucella suis
atgaaacgcgctctgtttcttgcccccttcttcttcgccctgcttgccggttgctcgcctgcggaaaagcaggaacccgccacacctttcgcactgacggaaaatgccatggggcgctattgcgggatgaacgtgctggaacatccaggccccaaggggcagatcatcctgcggccgaccaacgagccgatctggttctcgtctgcacgcgacacgctcgccttcaccatgctgccggaagagccgaagaatatcgccgccatctatgtgtccgatatggcaaaggcgcccaactgggacgagccgggtgcgaataactggatcgatgcacacaaggctttctttgtgatcgaaagcggggccgtgggcggcatgggcgcgaaagaagccgtgccgttctctgaccgcgccgctgctgaacggtttacagccgataatggcggccggatcgtcagcttcgatgagatgccgagggattacgtgctgggcgagcagacggcccagcatgtggaaccggacggcgaaagccatatgcat

SEQ ID NO: 161 - DNA, 789 bp, Brucella suis
atggcccgctcagcccttctcaacgtaatggtacaggccgccatgaaagctggccgcagcctggcccgcgatttcggcgaagtccagaacctccaggtttccctcaaaggccccggcgattatgtcagccaggcagaccgccgcgcagaacagatcgtctatacggaattgagccgcgcccgcccggatttcggcttcctgatggaagaatccggcgaggtcgaagccaaggacggacagcatcgctggctggtcgatccgctcgacggcaccacgaatttcctgcacggtattccggtttttgcagtctccatcgcgcttgagcgtcaggggcagatcgttgcaggcgtcatctacaatcccgccatgaacgagctttacaccgccgaacgcggcggcggcgctttcctcaacgatcgccgtttgcgcgtcgcctcgcgcaacaagctggtcgattcggtcatcggcaccggcattccgcatctggggcgtggccatcacggccattatctcgtcgaactacgcaatgtcatggcggaaaccgccggtattcgccgcatgggcgcggttgcgctcgacctcgcctatgtcgcaggcggccgcctcgacggtttctgggaagagggtatgcacccatgggatctgggcgcgggcatcctgatggtgcgcgaggctggcggtttcgtctccgataagaagggcgggcaggacatattttccacccagtccatcgtcgcaggcaacgaagccatccacggcgcgctcctgaagacactcaagaagccgatc

SEQ ID NO: 162 - DNA, 1329 bp, Brucella suis
atgaaaatcggcatcatcaatacaaagatccggacggtcttttcggctttcgcgtgtatgattgccgcaagcctcgtctgcaccatgccggcgcgcgctgtcgtggaaatcaacatcaacaagggtgtgatcgagccgctgccgatcgccatcaccgattttctttcggctgatcagcttggatcgaatatcacgtcggtcattgcggccgatcttgagcggtccggcctcttcgcccccatagacaagggcgctttcatcgaaaagatctccaatccggatgcggcgccgcgtttcgaggactggaaggtcatcaatgcgcaggcgctcgttacgggccgcatcaccaaacagcccgatggcaggctcaaggctgagttccatctctgggatacgtttggcggccagcagatgatcggccagcaatttttcaccacgccggataactggcgtcgtgttgcccatatcatcgcggacgccatctatgagcgcctgaccggtgacaaaggctatttcgatacccgtgtcgttttcgtggatgaatcaggcccggcgcagaagcgtgtgaagcgccttgccatcatggaccaggacggcgccaatgtgcgttttatttctgatggccgcgccctttcgctgacgccgcgcttctcgccgaaccggcaggaagtgacctatatgtcgtttgagggcggctcgccgaaggtctatctgctgcaacttgaaaccggccagcgcgagcttgtcggcaacttccccggcatgactattgcgccgcgtttttcgcccgacggccagaaggtcgtgatgagcctgctgcaggatgacggcagcgccaatatctatacgatggatctgcgcaatcgcacgaccacgcgcctgaccagcagccaggcgatcgatacgggcgcttcctattcgccggatggttcgcagattgtcttcacatccgaccgtggcgggcgcccgcagctctatgtcatgggtgccgatggctccaatccgcgccgtatctcgatgggtgacggcagctattccacgccggtctggtccccgcgcggcgatctcatcgctttcaccaagcaatcgcagggccagttctccattggtgtgatgaagaccgatggttcgggtgagcgtctgctgacatcgggctttcacaatgaaggcccgacatgggccccgaatggccgcgtgctgatgtttttccgcaaggcagccggtgcaggcggcccgaaactcttcaccatcgacctgaccggccgtaacgaacgccagatccagacgccgaattttgcttccgacccggcctggtcgccgctgctcgaa

SEQ ID NO: 163 - DNA, 1599 bp, Brucella suis
atggcacctcgcgttctcgtatccgataagctttcgcccactgccgtccagattttcaaggatcgcggcgtggacgtcgattacctgcccgatctcggcaaggacaaggaaaagcttctcgaagtcatcggcgaatatgacggtctggcgattcgttcggccaccaaggtcacggaaaagctgatcgcagccgccaagaagctgaaagtcgttggtcgcgccggtatcggcgtcgacaatgtcgatattccggccgcttcgcgccgcggtatcatcgtcatgaatacgccgttcggcaattcgatcaccaccgccgaacacgctattgcgctgatgttcgctgtcgcccgccaattgccggaggccgacacctccacccgcgctggcaagtgggaaaagaaccgcttcatgggcgtggaaatcaccggcaagacgctgggtgtcgttggctgcggcaatatcggctcaatcgtcgccacacgcggcataggcctgaaaatgcatgtggtggctttcgatccgttcctttcggatgcacgcgcgcaggaactgggcgttgaaaaggtcgagctggatgaattgctggcgcgcgccgatttcatcacgctccacacgccgctcaccgacaagacccgcaatattatcaatgcgcaaacgctggcgaaaatgaaaccgggtgtgcgcattgtcaattgcgcgcgcggtggcctgatcgtggaaaaggatctgattgcggcgctgaaatccggccatgtggcgggtgctggcattgacgtctatgaaaccgagcctgcaacggaaaacgagcttttttctctgcccaatgtggtctgcacgccgcatctgggggcatcgacttcggaagcacaggaaaatgtcgcacttcaggtggccgaacagatgtcggactatcttgtgaagggcgccgtctccaacgccatcaacatgccctcgatcacggcggaagaagcgccgcgcctgaagccgttcgtgaagcttgccgatgtgctgggcgcttttgtcggtcaggtcacggatgagccgatccaggaagtggaagtgcttttcgacggttccaccgcctcgatgaatacgcgtgcactgctcagtgctgctctggccggtctcatccgttcgcaggttgccgacgtcaacatggtttccgcgccaatcatggtgaaggagcgcggcatcatcgtctcggaagtcaagcgcgacaagtccggcatttttgacggctatatcaagctgacggtgaagaccaccgtgcgcgaacgctccattgcgggcacctgtttctccgatggcaagccgcgttttatccagatcaagggaatcaatctcgacgccgaggtcggcccgcatatgctttatgtcaccaacaaggacgttccgggcatgatcggccttcttggcacgatatgcggcaaacatgatgtaaatattgcgaatttcgcgctcgggcgcgaccatccgggtggcgatgcaatcgcaatgctttatgtggatgaacagattccacaggcagcccttgacgaactgatggctcaggaggcaatcagggcggcgacaccgcttgagttcaacgtcggggaagtt

SEQ ID NO: 164 - DNA, 1431 bp, Brucella suis
atggtttctgtcccggcctcgcgccctgcaaaccgcctgattgcgggagaagaaaagcgcttgcagcgggctctggcccgcacaggcctctggctgcgcgatcatcagcgcgcaatccgcgccattcagtgggtgattatcggggcctatgcgggattgctgattatcccgctcatgacaccgatgcccgatcgcctgtcccatatctggtccaatgcggttctgtttgcccagttcatgttctggggcatctggtggcctttcgtactggtgtccatggtgctgatgggccgcacatggtgcggcgtattctgcccggaaggtgctctggcggaagctgccagccgccacggctatggtcgcgccaccccgcgctggatcaaatggaaaggctggcccttcgtcgcttttgcctgcaccacgatctatgggcagatggtgagcgtctaccaatatccaaagccggtcatcatcgtgcttggcggctccacgcttgctgcaatggctgtgggcttcctttatggccgtgagaagcgcgtctggtgccgctatctctgcccggttagtggtgtgtttgccgtgctggccaagcttgctcccgtatattaccgggtggatcgcgagacgtggaacgattgggagcgcaaaccgggcacaccgatgccgcatgtcaattgcgcgccgctggtgccgatcaagacaatgcgcggggcttccgcctgccacatgtgcgggcgatgcgcgggtttcaaggatgctatccagctttcggggcgctcgccttcttatgaagtcgtcgaggttgcaggggatgcccccaaaccatgggaaagcctgcttatcatttttggcatgatgggcatagccgctggcgcgttccagtgggcatccagccctatctatgtcgatgtcaagcaatggctggccgaatggctggtggagcacggcggcatcgccatgatccagccgcagcttccctggtgggtgctgaccaattatcccgatctcaacgacatgatgacaccgctcgatggtggcttgatgatcgcctatattctcgctgtcgcttgcattaccagcattgcggtgggtgggcttgtggcaatcgccaattgggcgcttggcggctggaaggcggcgcgcttccaccatctggtgcaatgtcttatccccatggcggggtgcggcgtgtttctcggcctgtcggccacgaccgtgacacttttgaaagcggaagatgtgcctctggcttttgtgagcgacctgcgcattcttctgctgttgggcgccgtcgtctggtcggcatggctcttgtggtcggtcgcgcgccgctacacatcttcgatggctcgcaggcttgttgcgaccgccatgggggcgcttgcgacgggtgtagccgcattcaatttggtattgttgttctggatctgg

SEQ ID NO: 165 - DNA, 1758 bp, Brucella suis
ttggtgaagcgtatcaggaaccggaaacaggcgctgctgattgcggcattgccgcttcttgtcttttgtggcccgccacaagccatggcgcaggatgtcagtgccgcgcgtccggcagtgcttccccttcccggcgaagccgaaggagccagtatcggctccatttccgttcgcctgcgccaatccagcggcaatgcagaacgcgacgccgccgcgcaggaagccgcacgccgcatcaccaaatccctcgacggatcggaattcagcgccatcgaagccggcgaaattattgccgaactcattcgtcaggaagcaatctccgacgccagattttatctgctcccatctgcctcgcaaaccggcgtcaaactggtttttgaaatcgacgccacttcaggcaccaagacgaagcaagagccaaccggcctttttctcggcgacacatcctcctttccagtcctctatgaaagcaaccgcgcagtactgagaaccattatcggcgccggattgggcggctattccgatgtgaatgcatggttcggaaggccggatctgttcaatggcaggaatccgcttgctggcaaattgccgggccgctcaaacacatggaacgaaggctatctggagcttggcgtcggcggcgccgcgcaattgggcgaaagcaacctctatgggttcggcgctgtcaccggcatgttttcctggaccctcggacaggatatttttcgcgatgacgatcgcggcttcctcgacttcgagaaagcctatgccggtgtgctctatgtatcgccgaagaaccggaaaaaccatttcaaattctcggccgggcggcaggcttatacactcaatgacgggtttctggtgaatgccgttcgtggctccaccagcgccggcgaacgcggcgggctttatctcggcccccggctcaccaacgacttttcggttctggcgcaaggtgcttatgatcgctggaactatagtgccttcttcatcgatcccaacgaagttgaatcgctggaatccgacaccacttttctcggcgcaaatctgcgttacaacttcaccgacaagttttctgccgacactacattaataacgatccccaactcgaaaacacaattttccaccccctctggcagattaatcccgcgtgaagggctgaacacggtttcaggccatttgaaatggaccagtgctttcgagatgcccggcctgtggctcgaaggcgaactggcgctccagtttcaccccggtgaggatgtgtcggcctgggcctattacggaacagtcggctatctcatgaaagagattggctggacgccaagtctttcctatcgctacgcctatttttcgggcgacgatccgaactcatcgcgttatgagcggtttgatcccctgctctcgaccgggcttggcatctggttgcagggtgtttcgtttggaaagatcaccaccaattccaatcttgagacgcaccgtgtccagttcaatgtcgcacccgacgagcggctgaacctgacattcgactactatctgctgcgtaccccccaattgaacaatctcggttcaaacccggcgctttcgacgctatcgtcgcacgatcttgggcaggaactttcactttcagcacgctggagcgcgaccaaaagcctttacctgcaagcacttgtttcacatgccattcccggcacggcgctacgcgatatcggtgccgaccggaactggacgacttttcaggtcagcctctacgggggcttt

SEQ ID NO: 166 - DNA, 1233 bp, Brucella suis
atggatggttccgtggtcgtcagccagttgggcgcgcggatgcactatgcggtgcctcgcatttttgccagccgcgaaaggcttgcccatttctataccgatatttgcgccttgcagggttggccgcgttttttcaatggcctcccgccaaccatgctgcccgcttcggtgcgcaggcttgtggggcgaattccgcagggtattccgcaagagcggatgacgacgtttccgggtttcggcctgcactctgccgtgcgcaggctttccaaccagactgggcccaaatcgactgccaatgcaatctgggcgggcaagaccttcggcaaactggtggccgaaagcggctttcatggtgcaagtggcctttatgctttcagcggtgaggcgctggaactgctggctaccgcaaagcgccagggattgtggacggctgtggagcagatgattgcgccgcgcatggtggtcgatcaactggctcttgaggaagaagcccttaatccgggctggcagcatcctgttgcaaacgatgcacatgccgatgtttttgccgcacgcgagcaggctgagtggcggattgccgatgtggtggtctgcccctcggcattcgtggcgcaccatttggcagaaatgggctgtgcagcggaaaaaatcgtcgtggtgccatatggtgtcgatgatcgctttctgctcgccccctgtcctcgtctgccggggccattgcgggtgttgacagtgggagccgtaggcttgcgcaagggcgcgccttatgtcggggctgtggcccgtatgcttaaaggtgaggcgcagttccgcatggtcgggccaattgagattcaagcggaagcgcaggtaaaacttgctgcaagcgtggaactgaccggcgctattccccgcagcgaaatgcgcgcgcagtttaaatgggcggatgtgtttttgctgccttcgctttgtgaaggttccgccacggcggtttacgaagcacttgccgccggcttgccggtcatctgcaccccgaatacgggaagcgtcgtacgccacggcatcgacggttatatcgtgccgatccgcgatgtgcacgaaacggcgcaaatcctgcggcaacttgccgataacccggcactgctcgcgcggatgagcgaaagcgcgcgggagaggggtgctgatttcaccgtatcgcgctatggcgaacggctggtcgcagcgctggcccgtcacgacaggaggcaacgcgca

SEQ ID NO: 167 - DNA, 1287 bp, Brucella suis
atgacagatttttacagcctcatcccttcggcacccaagggccgcttcgacggcattgaacgtgcgcatacggccgaagacgtgaaaaggctgcgcggttcggtggagataaagtattcgctggccgaaatgggtgcaaaccgcctctggaagctgatccacgaggaagatttcgtcaatgcgctgggcgcgctttccggcaaccaggccatgcagatggttcgcgccgggttgaaggcgatctacctttccggctggcaggttgcagcagacgccaacacggcatcagccatgtatcccgaccagtcgctctatccggccaatgccgggccagagcttgcaaagcgcatcaaccgcacgctgcaacgcgccgaccagatcgaaaccgccgaaggcaaggggctttcggttgacacatggttcgcgcccatcgtggcggatgccgaggcaggctttggcggcccgctgaatgccttcgagatcatgaaagcctatatcgaggccggtgctgcgggcgttcactttgaagaccagcttgcttcggaaaagaagtgcggccatctgggcggcaaggttctgatcccgaccgcagcgcacatccgcaacctcaacgcggcacgccttgcagccgacgtcatgggcacgccgaccctcatcgtggcgcgcacggatgcggaagcagccaagcttctgacgtcggatatcgacgagcgcgaccagcctttcgtcgattacgaagcaggccgcacagcggaaggcttctatcaggtgaagaatggcatcgagccatgcatcgcccgtgcgattgcctatgcgccttattgcgatctgatctggatggaaacgtccaagcctgatctcgcacaggcccgtcgcttcgcggaagccgtgcacaaggcgcatccgggcaagctgctcgcctataactgctcgccgtcgttcaactggaaaaagaacctcgacgacgcgacgattgccaagttccagtgcgagctgggcgcgatgggctacaagttccagttcatcacgctggctggcttccaccagctcaactacggcatgttcgagctggcacgcggctacaaggatcgtcagatggcggcttattccgagctgcaacaggcggaatttgcagccgaagccgatggctacacggcaaccaagcaccagcgcgaagtcggcaccggctatttcgatgccatgtcgctcgcaatcaccggcggccagtcttcgaccaccgccatgaaggaatcgaccgaaaccgcacagttcaagccggctgccgag

SEQ ID NO: 168 - DNA, 1014 bp, Brucella suis
gtggtcaatatctataactggtcggattatatcgacgattccatcctcaaggacttcaccaaggagaccgggatcaaggtcgtctacgacgtctatgactccaacgaaattctggaaaccaagcttctggcgggcggcagcggctacgacctcgtggtgccatcgggcgaatttcttgggcgccagattcccgcaggcgtgttcctgaaactcgacaaggacaagctgccgaacctcaagaatatgtgggatgagatttcgacccgtgcggcaacctatgatccgggcaacgaatattccgccaattacatgtggggcacgaccggcatcggctacaataaggccaagatcaaggaagcgctcggcaccgacacgatcgactcctgggacgtgcttttcgatccggaaaaaacggcaaagctgaaagattgcggcatttacctgcttgattccgccagcgaaatgctgcgtccggcgctgaactatctgggtctcgacccgaactctccgtcgccggacgatttgcagaaggcacaggatctctatctcaagattcgtccgaatatccgcaaattccactcgtcggaatatatcaatgcgctcgccaatggcgatatctgcatggctgtcggctattccggcgatattttccaggcccgcgaccgcgccgaaaaggcgaagcagggggtggagatcggctattcgatcccgaaggaaggcgcgctgatctggtttgaccagatggcgatcccggccgatgccaagcatgtgccggaggcgttggaattcatgaattatatgatgcgcccggaggtggcggcaaaggcgtcgaactatgtgttctatgccaatggcaacaaggcgtcgcagaaattcatcgacaaggaaatcctcgacgacccggaaatctatccgtccgacgaggtgatgaagaagctgttcgtgccgacgccatatgacacgaagacccagcgtgtggtcacacgcgcctggaccaagatcgtcaccggccag

SEQ ID NO: 169 - DNA, 819 bp, Brucella suis
atgcagaacaactggtcccgcttcaatatcgtatcggtggcgctgggttttgcctttctctacctgcccatcgtgcttttggtgatttattcgttcaatgaatcccgtctggtcacggtctgggccgggttctcgaccaaatggtatggcgaattgttccgcaatcaggccctgatggatgcggcctgggtgacgatccgcgtcggccttctgtccgcgacgatcgccacggttctgggcacactcgcagcccttgcgctcacgcgttatacccgctttcgcgggcgcatcctgttttcaggcatggtctatgcgccgctggtcatgcccgatgtcatcaccggcctgtcgctgcttctgctcttcgtggcgatgaatttcgaccgcggtttctggacagtgacacttgcccatattaccttttccatgtgtttcgtggcggtcgtggtgcaatcgcgcctcgtcagctttgaccggtcactggaagaagcggcgatggacctcggcgcgccgccggtcacgaccttcatgaaggtgacgctgccggtgatcctgcctgccgtggtttccggctggatgcttgctttcaccctgtcgctcgacgatctggtgattgcgagtttcacctccggtccgggggcaaccacgctgccgatgaaaatctacagccaggtccgccttggtgtgacgccggaaatcaacgcggtctgcaccattctcatcgcgctggtgacaacgggcgtcattatcgcctccgtcatcaacaagcgccgtgaagtgcagcgccgccgcgacgaacaggcagcattccgtatggga

SEQ ID NO: 170 - DNA, 1125 bp, Brucella suis
atgacaagtctttcctggatcacgcttctggcttttgtcctgtcggtggccttctccttctgcatggcttttcgtgggcttgcgcgcgaatggtatctgatcgacctgcccgattaccgcaaaagacatgaaggtgccgtcccgctttgcgggggtaccgcaatcttcctgtcctttttcctgacagcttcactggtaaaagacggtgcgggacatgcagatgcactggtgatgatgccggggctgacgatcatcctcggcacgggggtgctggatgatcgcttcaggctgccggtgacgccgcgttttgcaatccagctttttgcagcggttctcatcatcgccatggcgggggtacagcaggtctttctggacctcaacccctcctttatcgacgatagcggcaatatatttgcctttgaacggctgggcggcccctttctattgcttttcgccgtggccttcatcgtcggccttgtcaatgcggtcaatatgagcgacggcgtggacgggcttgcaggatcggcaagtgcagcctccttcttctggctcgccgttatcagcttcgacatcggcgaacaccggctgggcctgcaatcgctggcgcttgtcgccgcctgccttggtttcctcgttttcaacatgcgccataggtggcgggcaaaggcaagcctctttctgggtgacggcggcagcacgcttctgggggccgcccttgcgggaatggtgttgatcctctccagcggcaggcaagcaattgctttccctgtcttgctttggatcgtgattgtgccggtcctcgatacgcttagcctcatcgtaagaaggctcgcggcgcggcgcagccctttttccgccgaccggcagcatctccatcacctgctgatggatgccgggctgacctgcgggcagaccgcaatcatcgtcatggcgctcaacctgattgcaggcgcaacagcctatacggctattcgcctcgacctgcccggctggctgatggcgctggcgcttctcgcaccggccatggcgcacctagccttcatcctgcgttgcaccagaggtccgcgcatcgccgatcccgcgcaggcggcaaaaccgaacatcacctttccggggaccacgaca

SEQ ID NO: 171 - DNA, 1047 bp, Brucella suis
atgaacaggcaccgcaaagggttcatcatcgccacgcttgagcaatatgccgggctgatgtccaatttcctgaccatggtggctgtgtcgcgccttctgggccctgccgaaaccggcataggggtggtgggcctcggcattgcggctattttcttctccatgcgggaatttgcgagcgccgaatttctcatcaagatcgataaggtcgaggatagggatgtgcggacatcgctgactttggtgatgctggcgaccctgatcctcgctgccggtctttatctggcgcgggattatctggcgacttcctatcgcgatccctcgctcatgctattcctgaatatcaccattgttgcggcgctggtggagagcgcgtcctttccggtggtggcgctgctgcggcgggaaatggccttcggcgttctggcgtggatccgcacggcgggatcgacgtgcggggcggcaacgaccatttccatggcctatctcggctttggccatatgagcttcgccttcggggcgctgtccgccgcactcatcaccaccggctttgccttcaaggcctatccgatgaaacatctgctgcgcccatctctggcgagcatcggcaccgcctggcagttcggcctttatcttggcagcagtgcgggcctgaacaagatttgcgaaacatttccgcagcttatccttggaaagttcatgtctgcggcgtcggtcggcatctatagccgcgctggcgctgtcagcggggttccagaccgcatattcctgtcgtcgatctttacggtggcttttcccatgttatcggcacaggtgcgcgagggggccgatatccgtcgctcctacgttcaggcgctttcctatatttcggtggtctattggccggcacaggcggtggtggcgcttctggcctatccggctgtgcatatcattctgggtaagggctggatggaagccgcgcccatcgtggcgctcagcagccttgcaagcctgttctggttcccggtcattcctcacctatcccttgttgatggcactgggcgcaaatcg

SEQ ID NO: 172 - DNA, 780 bp, Brucella suis
atgaagtgttatcttattaatctcgacaagagccgggatcgcctcgaattcatggcgtctcagtttgagcgcctcggcgcacaattcaagcgtgtggaagccgtaaatggacgagccatgtcgccgcttgaactggcttcctttactcaaataagtaaagaatggcccgctcctttgtcgcctgccgaaattggctgttttctttctcatcgcaaatgccttgaaaagattgctgccggcgaagatgcctatgcggcagtctttgaagatgacattcgattgagccagggttcctcgcggtttttggcttcagatcactggatacccaagcaagcggatatcgtcaaaatcgacgcttacggacatgaggtattgatttccaaccctgttaaaaatgaaggcccgtattcgatttcccggctgcgctcgcgacacttgcagacgggcggatacgttgtttcgcgcgaagcagctcgcaagctccttccattgatggaaaaagcctcagcgccggtagaccatttcttgttcgatcccaatgatgggccattcaacgattttgaaatttatcagatttcccctgcaatttgccgccagtcgggaatggaaagcacaatcggtcagaatcggcgccccaaacagcgcccctctcttttgggcttggtctggcgtgaagctaaaagattggttatgcgaacccgtcgcaatctaaaaggtttcatagccaacgtcaccaaaacaggccggtgggggcccattccatttgatcgggatattgcg

SEQ ID NO: 173 - DNA, 1377 bp, Brucella suis
atgcgggggcaggcaattgcgataaactggcagcggtatatctcatgcccccgatataccggaaccaggcttcgagggagcttctcccccgaagtccggttcccggaaccgtcacggggcaagcccggtgtccagctaaagagccttgccatgaaatgtgcctacttcgtgcgcccccatattggcggaacctattcggttttcacacggttgcgtgcttcgcttgcccggtctggcatggatctgcgctggcttgcagccggtgcgccgggcgaagcggtggccgtgccgcgcgagcaggggctggaagatgccatgcgccggggcgacatcatagatcgcatgggcgttctggatgaagaaacccgcgccaaacgctttgtcgattttcttcgcgaaaacaggtttgaagcagtttttgtgaatgtgctttctcaccgtttcgagaccaatctcgtgcgctatcttcccgcggatattctgcgtgtgatgatcgttcacagcatcacgcccggcacctatgcggcggcgcaggcgatccgcgatcatgttcacgcgacggtcggtgtttccaaacggtgccgcgacgatctggttcgtggctatggcttcgaccccagccgcacactcgtcattcccaatgggctgacgcccgatccgcatgtccggtgcgcagcgccggagattgaaggcggacggctgcgactgctctatctggggcgcatcgatgacagttccaagggggtcttctggctgcccgcgatcctgcggcggctgcaatgcgattatcatctgacggtggcgggcgacgggccggacctcgcagccttgcgcatcaagctggctccctttggcaacaggatcagttttaccggctgggtcgcgccatccgatgtttcatggctggtgagccagcatgacgtgatggtcatgccatcgcgctatgaaggtttcggctcgacgctgattgaggcaatgagccagggatgcccggtggtggcgtcgaggataccgggcgtgacggatactatcgtcaccgatggtgaagacggttttctgtttccgatcggcaattgcaggcaggcggcgcagcagatcgagaggcttgccgccgatccgcgcctgcgcgtggcgatgggtgcggcaggcgtgcgcaaggtgatcgcggaattcgacagcgaaatcgtcgggcgcgcctatgcgggattgctccagcatctgcgtgaaaatcgcccgcccattacgccgcctctggctctttcgcagtggtccatgcccaatgccctgcacaaaaacctgcgcagccgcttgccgcagccggtcaagaactggctgcgccgcgtgcgggagcgcgtgcacacggtatggtcgacggcc

SEQ ID NO: 174 - DNA, 1137 bp, Brucella suis
atggatcaccacgcgggcattcccgatctgcgccttcaccgcatcctcgatgcggtcacagatgacatgtgcatcgccgacgctcatatccgccgccacgacaaggtgaaactcgatgaaggtcacgcggcccgcaatgcgtgtcttgagatcgtgaacctcgatagccccacccgcattggccgagataacattgcggatacgcatttcctcggtcggctcgaccgcaatatccatcagcccctggaccgaggaattgatgacgtgccagccctgccagagaatattgatggccaccagaatggcgagcaatggatcgagcaccgaccaaccggttaccaccacgccgacaaggccgaccagaacgcccgcagaggtgaacacatccgtcatgatgtgcttgccgtcggcttccagtgctggcgagcggaatttgcgtccggtgcgaatcaacaatgtcgcccacaggccgtttatggcagcagcagccatgttgatgccgagacccagccagggttcatcgagcattcgcggtgtttccagtgcaaaccaggcctcgcggaagatcaaaagtgcggcgaccgtaatcagaacgccctccaccacagccgaaatatattccgccttgtggtgcccgaagggatgattgtcgtcggcgggcagatagctgacccgaatggcccaccacgcgccgattgccgcaatcacattgacaatcgattcgagtgcatccgaataaagagccacggagcctgtcagcgcataggccacatatttcagccccatcacaccgaaagcaacgggaatagaccaagcggcaagccgccgaacctttgcactcgcgtccataatgaacctttgattatcttgatctttcccgaaaccgggacgggcctacgcgtaagcccttcattcgttcgccagagcggttcctgtttcaacgaaatcgctggaaacgctctaactctttgttttgtcgcattatccggcacaaaaccgtttcacacttttgctggaaatgctctatatcccagaaaaacaaaacttcgtaccctgttttttgcgttccagcatttagagcgcatcccgaaaagtgtgaaacggttttcggaaaagatgcgcgtcaaaacaaaggat

SEQ ID NO: 175 - DNA, 1047 bp, Brucella suis
atgtccgtatccaagaaatatatccgcggtggctttgcaatcctgattggccttttgggtgttttcatcatcttgtgggcttggcagttgccgccgttcaaacacagtgtcgaaacgaccgacaatgcctatgtgcgcgggcaggtgacggttatgagcccgcaggtttcaggctatataaccaaagtcaacgtgaccgattacgagcaggtcaaaaaaggcgatctgctttttgagatcgatagccgcagctatcagcaaaagctggatcaggcgcttgccgcgttggacagcaagaaggcggcacttgccaattcggagcagagccagcgttccgccgaggcaacgataaaggcgcgagaagcccagatatcgggggcaaaggcggcgcttgaagtggcaaaagccaatacggttcgtgtcgatgccctgttgccgcgcggtgtgacgacacaaagctctgccgatacggcacatggcaatatgcttcaggcgcaggctgcggtcgatcaggccgaagcggcactggccgttgcgaaggaggatttgcgcaccatcatcgtcgcgcgtgaagggctcaatgccgacctgcacaatgcagaggctgcggtggcgctggccaggatcgacctccagaacacgaagatccttgctccaagggatggcaagcttggcgaagtcggtgtgcggcttggccaatatgtcaccgcaggcacacagcttgtttcgctcgtgccggccgtcaaatggatcatggccaatttcaaggaaacgcaactttatggcatgaaggtcggccagccggttacgctgacggtcgatgcgctccgccatgcggaactgaaggggcatatcgaggctttttcaccggcaaccggctctgaattcagcgtcatcaaatccgacaatgcgaccggcaatttcaccaagatcacgcagcgcttgtctgtccgcatttccatcgacgaagggcagccggatgctgaattgctggcgcccggcatgtcggtggtggtgcgtgtcgatactgcctccacgccggataaa

SEQ ID NO: 176 - DNA, 504 bp, Brucella suis
atggtgacggccagaatgcgcgatgccgccgtgcttgttccggtggtggaccggggggcggaggcaacgctgctgcttacccggcgcaccgatacgctgcgcaaacattccggccagatcgcctttcccggaggggccatcgacccggaagacggaacggcggaacgggcggcacttcgcgaggccaatgaggaaatcgggcttgccgcagaccgtgcggagatcatcggcaacctgccgcgctatctcaccggcagcggcttttccatcacgcctgtcctggcggtggtgaaaacgccgttcgatgtgcatcccaatccggacgaagtcgccgatatttttgaagtgccgctttccttcctgatgaatccggccaatcatcgccgcgaaagcaggctgttcaacggcaaggaacgcttctattacgccatgccctatcacgagcgattcatctggggggcaaccgccggcatcattcgcggactttatgaaaggctctacgtt

SEQ ID NO: 177 - DNA, 597 bp, Brucella suis
atgaacaatgctctcctcgattccatcaagaagcgccgtacgcaatatgctctgggcaagacccttccccatgcccgggaagatgttgccgaactgatccgcgaagcaatcaagcacacgccctcttctttcaattcgcaaagttcgcgcgcggtgatcctgtttggccaggaaagcgacaagctttggaacattgtgaaggaaacgcttcgcccgatcgttccagccgaagctttcccgcagaccgaagccaagatcgacggctttgctgccggtgcgggcacggttttgttctatgaagaccagagcgtcgtgaaaggccttcaggaaaatttcccgctttatgccgataatttcccggtctggtcggaacagtcgggcggcatggcgcaacatgcggtctgggccgcgcttgccaatgcgggcatcggcgccagcctccagcattataatccgctgatcgacgcggaagtagccaagacctgggatgtgccgagcaactggaaactccgcgcccagatgccgtttggctccaatgagcagcctttcccggaaaagcagttcatgaacgacgctgaacgcttcaaggtgttcttc

SEQ ID NO: 178 - DNA, 1398 bp, Brucella suis
atggccgcggccatgcttctgcccgccgtaatcgatctgcgtgacggaagcgacgactggtcggttttcgtgcgctctgctgccgccacgggtgctctttccgcgcttatttttttagcgacacagaaccagaccatgcgtttcacgccgcgtctgggctttcttctcaccgcctgcctctggctgacggccgcgtttctgggttccataccgctttacttttcgcatctgccgatcagctatgcgaccgccttcttcgaggctatgtcgggcgtaacctccaccggagcgaccgcgctgaccggcctcgacaacatgcagcgcggcattcttctgtggcgctcgctcctgtgctggatcgggggcgtcggcttcatcggccttgcgcttttgatgttgccctccttgcgggcaggcggccttgcgctttttcacatggaaaattccgacaaatcggaaaaaatcctgccgcgcatgaaccagatcgcgctcggcatcatggccgcatatctaagcctcacggctgcctgcacggttgcctatttcgctgtgggcatgagcgttttcgacgccatcaaccacagtctcaccaccattgccactgcgggcttttccacccatgacagttcgatcggcttttatcagggcaaccggatgctgctcgtcgtatccaccatattcatggcgctctcagccctccccttcgtgctttacatcaaggctttcatgccgcggcgcatggaattactcaccgacccgcaggtgaaactcttcttcaccatcatcatcgccttcagcttcatcctcgccatcgtcctgcgcctcagcaccaatatacctttcggcgatgccctgatctcggcctcgttccattttgtctcggtgataacgacgaccggatatgcaaccgaggattattccctctggggaccgccagcgattggaattttctttctcgcctccttcctcggcggctgtgcaggctcgacttccggcggcatcaaaatgaaccgcctcatcatcctgtggagcctgacgcaggccaatcttgcgcgcctcatcatgccgcatgccgtcatcaagattcgctacggctcatcggaagtttcgggcgatatcgcgcagaatgtattgctttacctgtttctttacagcgcctcgctcattatcggcgcggtgctgctcgcatccttcggccttgatttcgtatccgccttcacgggttcgctgacggcattatcgaatgtcggtcccggccttggcgacaagatcggccccgctggcaatttctccaccgttcgcgatcaggcgctttgggtgctctcattccttatgctggcaggccgactggaactcgtcaccatattcatcctcttcacacgctcattctgggtgcgt

SEQ ID NO: 179 - DNA, 450 bp, Brucella suis
atgcttcgccaggagatttcccgggccctcacaaccgcgctgaaggcgcaggagaagcgccgcatgtcaacgctgcgccttgtcatggctgccgtgaaggaccgcgatatcgccaaccgcaccgctggcaaggacccggtcggggacgaagaattgctgggcattctgggcaagatggtaaaacagcgcgaagaatccgcgcgcatctatgaagaaggcagccgtctggaactggcggaagccgagcgcgaggaaattgccattatttcggaattcctgccgcagcagatgacggaagacgaaaccaagaaggcctgtgaggatgtgattaccgaaatcggcgcgcagggccttcgcgatatgggcaagtgcatggcgcttttgaaggagcgctatgcaggcaaaatggactttacccgggcaggcgcacttgtaaaatcgctgctgcaa

SEQ ID NO: 180 - DNA, 177 bp, Brucella suis
ttgcagtccgtcctgcgggagggaggaactcttgcggacggtggccagaggggaaaccgggtgacggaaatatccgcgcccggtttcataatttcaagccctgttctgcctgttgcggatggccgcccggccattggcgtttgcgcgaaggcgtttgcttctgcgccgcatttggcc

SEQ ID NO: 181 - DNA, 927 bp, Brucella suis
atgccgaaaacgacggggggtggcgtggaggcgcatgtgaagggaatgggcttcatggtcctttccgtgctcatgttgccactgatggatgccatcggcaaatggcttgccatgatggacgatttgccgcctgcgaccgccactttcatgcggtttttcgtacagtgctggctgatgttcttcatcatccttgttgtgggtggcttgcgggcgctggccacctcgcatctgatgggcaatctgctgcgcggcgcgttgatggggttcggcggcctttgcttctttacggcggtcaaatatatgccgcttgccgacgccatggcggtgttttttgccgagcctttgatcctgacgctgttttcagcgcttttcctgaaggagaaagtcggctggcggcgcttcagcgccgttggcatcggcctcatcggcacgatgatcgtcattcagcccagtttcgagattttcggcatggtttcgcttctgccgctcgcaaccgctgttacttttgcaatttatctgatcctcaaccgcaaatatggcgcgaaggaaagcccgcttaccatgcagttgtatgcggggctgggcggatggctgttcgtgggggtggcaatgctgctcgctgccgataggagctttgacgatatgcatttcgccctgccgcacagcgtacagccctggctgctccttttgcttttgggcaccatcggtacggtcagtcacctcatcgtggtgcaggcattcaagcttgcgcccgcatccatgctggcaccgtttcaatatctggaaattgtgaatgccgttctggtgggactgattatcttcggcgattttccaacgccatccaaatggctcggcatcgccatcatcattggctccggcttttacgtcttcatgcgtgaaagcaagaaaaaaacaggacggatctca

SEQ ID NO: 182 - DNA, 93 bp, Brucella suis
atggcgtcaatcgaaatcttgatcactcttgattattcctgtgcgatcctgtccctgcacgattttttgcagaagacaggttttcggggaaaa

SEQ ID NO: 183 - DNA, 1752 bp, Brucella suis
atgaccggcgcagaaatggttatccaggcgctgatcgatcagggtgtggaaagcattttcggctatcccggcggcgcggtgcttccgatttatgacgagctgtttcagcaggatcaaatccagcacgttctggttcgccatgagcagggtgcaggccatgcagcggaaggctatgcgcgctcgacgggtaaagtcggcgtcatgctcgtcacttcggggccgggtgcaaccaatgccgtgacgccgttgcaggatgccttgatggattccatcccgctggtctgcatttccggtcaggttcccacgacgctgatcggttccgatgcgtttcaggaatgcgatacgattggcatcacgcgcccctgcaccaagcacaactggctggtaaaggacgtcaacgacctgtcgcgcgtcctgcatgaagctttccatgtcgcatccaccggccgtcccggccccgtcgtcgtcgatattccgaaggatattcagttcgccaagggcatctatacgccgccggaaactgcaccgcgcacaagttaccgtccagttctcgacggcaatgccgaggctatttccgaggctgtccgtctgctcgccaatgccagaaagccggtcatttattcgggcggcggcgtgatcaattccggccccgcggcttcccgtctgctgcgtgagctggtcgagatcagcaatttcccgatcacctccacgctgatggggcttggcgcctatcccgcctccggcaagaactggctcggtatgctgggcatgcacggcacctatgaagccaacatgaccatgcatgattgcgatgtcatgctgtgcgtcggtgcgcgtttcgacgaccgcatcaccggccgcctcaacgctttctcgccgaattccaagaaaattcatatcgatatcgatccgtcgtcgatcaacaagaacgttcgggtcgatgtgccgatcatcggcgatgtcgcgcatgtgctggaggatatcgtccgccagtttcgtgctttcgaccgcaagccagacaggcaggcattgaccgaatggtgggggcagatcgaccgttggcgcgcgcgcaattcgctggcctatacgccgaacaaagacgtcatcatgccgcaatatgccatcgagcggctttatgaactgaccaggaaccgcaacacctatatcaccaccgaggtgggccagcaccagatgtgggcggcgcaattcttcggctttgaacagccgaaccgttggctgacttccggcggccttggcaccatgggctatggtctgcctgcggcgctgggcgtgcagattgcacatccggatgcgctggttatcgatattgcaggcgatgcctcgatccagatgtgtatccaggaaatgtcggcagcaatccagcataatgcgccgatcaagattttcatcctgaacaaccagtatatgggcatggtgcgccagtggcagcaattgctgcatggcaaccgcctgtcgcattcctacacggaagcgatgcccgatttcgtgaaacttgcggaagcatacggcgcgcacggcattcgctgcgagaagccgggcgatctcgatgccgccattcaggagatgatcgatatcgacaagccggtcattttcgattgccgcgtggcaaaccttgccaactgcttcccgatgatcccgtcgggcaaggcgcataatgaaatgctgttgccggacgaagcgacagatgaggccgtcgccaatgccattgatgccaagggccgcgcgctcgtc

SEQ ID NO: 184 - DNA, 570 bp, Brucella suis
atgaacgcacaaaacctagcttccggatcggcctatttcattgctgccgaaacccagacgcccgaaacccacacgcttgcagtcctcgtcgataatgagccgggcgttcttgcccgcgtcatcggcttgttttccggccgtggctataatatcgagagcctgacggtgtcggaaaccgaacatgagcgccacctttcgcgcatcaccatcgttacgcgcggcacgccgcatgtgctggaccagatccgccatcagcttgcgcgcatcgttccggtgcatcgcgtggtggatttgagccaccgcgccgccacgcttggccatgaacgcccgatcgaacgggagcttgcattggtgaaagtggctggaaaaggcgaggcgagggcggaaacgttgcgtctggccgatgccttcaaggctaaggtggtggatgccacgacggaccatttcattttggagattaccggaaaatcggccaagatcgaccagttcatctcgattatgaagccgctgggtctggtggaaatctgccgcactggcgttgccgccatgaaccgtggtgcggaaggcatg

SEQ ID NO: 185 - DNA, 915 bp, Brucella suis
atgctttacggtattttacttgcgtttgcctcctatgcagcattcgccgtcagcgacgcctgcgtcaagtttctggacggcacgctttccccctatgaaatagccttttacggcgctgtactgggtttacttgcaattcccttcaccaaaaaagccgatgatgcatggctcgacatgttccgcaccggcaatatcaaaatgtggctgctgcgcacggtcacggctgcaatgggcgtcatcggcagcgtggcagccttcacctatctttccatggcggaagcctttgcgctgatcttcctcctgcccgcctatgtgacaatcctgtccgtgatctttttgaaagaacaggtcggctggcgacgctggtcggcggttgctatcggttttgcgggcgttctggtggtgctgcgcccaggctttcgtgaattatccatcggccatctgggtgcgctggcggcaggcatgggcggcgcgacgagcatcatcatcttccgcgtgatgggaaagaacgaaaagcgcatttcgctctatacgagcgggctggtcggccccattctcatttgcggcgcgctgatggcgcccagctatcaagcaccgggcacgatgcagtgggtctatcttgcgggctacgggcttctggcggctcttgccaatatattgctgatgctggccgcgcagcgcgcgcccgccagtgccatcgcctcaccgcaatatagccagatgatctgggccatcctgtttggatatttcgtcttccacgaccatatcgacatgcccatgctgatcggcatcgtgctgattacgatttcaggtcttttcacgttcatccgcgagcgccagcgcggtgtggaaggcccgcccgccgtcgcggcagagccgacaccggctctggtccaagaaaaagcggggtatttc

SEQ ID NO: 186 - DNA, 1125 bp, Brucella suis
atgagcgaacccaattccgaaacgaaggccgcagcggcggcacccgccgatgagcggctgaaatcggccagcaccttgcagcgcctgttgagcaggccggaactgggcgcggtgtccggtctggttctcatcacgatcttctttgtttcaaccgccaattcggccatgtttaccgcagccgggatcatgaatttcatggccccggcctcgcagctcggtattcttgctgtcgcagcggcgctgttgatgatcggcggcgagttcgatctttccattgggtctatggtggcctttgccggcctgatttttgccgcagcccttgtcgtctggcaggtgccgcttgccgttgccattatcgttaccttgctgtttgcggcgctggtgggggcggtgaatgcgcagatcgtcatcagaacgggtctgccgtcctttatcgtcacgctggcctttctcttcatattgcgcggcctttcgcttgtcgggctgaagtgggcaaccggcggtgcgacgcagctgcgcggtgtgaaggaagcggtcagcggcagctatctggccggcctcttttccggcgatgcatttccggggcttttcgcatggctggcggaaaaggatttgattgcgaaatttccaaatggcgagccggcagtgaaaggtgtgctggtcgaggtgctgtggtttgtcgtgattgcccttgtcgcgacctatattctggtgcgcacccgcttcggcaactggatttttgcggcgggcggcgatgcgcttgccgcgcgcaattccggtgttccggtgcgccgcgtcaaaaccacgctcttcatgtgcacggccatggcggcagcacttgtggctatcctgactgttctcgatgccgggtccaccgatgcacggcgcggtttccagaaagaattcgaggcaatcatcgcggccgtgatcggcgggtgcctgctgaccggcggttatggctcggccatcggcgctttcttcggcgcgatcattttcggcatggtgctgatcgggctgacctatacgcagatcgaccaggactggtatcttgtgtttctcgggggaatgcttctgatcgctgtgctcttcaataatttcatccgcaagcgcgccacgggcgagcgt

SEQ ID NO: 187 - DNA, 1026 bp, Brucella suis
atgacgggttccggaaaccgcctaaccaaactgacgcttgcacttgtcctcggccttgttgccgtattcacggctgtcgatttcgctgaggctcgccgcgcgggcggtggcggttttggtagccgtggcatgcgcacctattctacgcctgcgccgacccgtaccgcgcccaacaatgcggctcccatcgaacgctcgatgacaccgaacacaggcgcgacgcaaacgacccgtcccgctgcggctggcgcacagaatgcggcacagcgttccggtggcctgttcggcaattttggccgttccatgcttggcggcctgctggtcggcggcctgatcggcatgttgcttggcaatggtctcggcggtcttgccggcatgttcggcctgatcctgcaagtggccgtcattgccctgatcgccatgttcgtgatgcgcatgtttgccaatcgccgccagacggcagcggctggcgcgggccatgccggggcgcagcctttcggcgcgagccagcctttcggttcggcgaaaagcgcatccggtcagggcagctttagcggccgcacaccatccgtcaatccgtttgagaccaaacaggctgcggctcccgtcgtggaggatgagccgatggatgtcggacaggaagatctggatcgttttgagcagatgcttgtggatatccaggggggctatggccgtgaggattatgcggcgctgcgcaagctcaccacgccggaagccatgtcttatctggcggaagaacttggcgaaatcgcatccggcggcaagcgcaatgaagtgaaggatgtgaagcttcttcagggcgatgtgtccgaagcctggcgcgaaggcaatgtggaatatgcaacggtggcgatgcgctattcggccatcgacgtgatggtggatcgcaccagcggcgcggttgtcgaaggcaacccgaatgagccggtggaaagcgtcgagctatggaccttcacccgcacagatggcggcgcgtggcaactcgccgctattcaaggcacggaa

SEQ ID NO: 188 - DNA, 864 bp, Brucella suis
atgaaaaagctccttgcacattccggccgggtttttctcaccctcgtcatggttgttatggcggggctgctcggctggcatctctgggattattacatgaatgcgccgtggacccgtgacggcaaaatccgtgccgatgtggtgcgcattgcgcccgatgtctcgggccttgtgagcgaggttctggtgcatgacaaccagacggtccataaaggcgatgtgattttccgcatcgaccaggcgcgctacaagcttgctttgcagactgctgaggccaaggctgacagcagcaaggcagcactggacatggccaatcgtgatcttgtccgctatcgccagctcaacgatacgaccgtgacgcgccagaaactggaacaggtagaaaccgcagaccagcaggcagaggccgcccaccgccaggccgaactcgaccgcgatctggctgcgcttaatatggagcgttcgtctatcaaggctccggtcaatggcgttatcaccaatttctcgcttcagccgggcgattatgtttcggcgggttcggccgttacggcgctggtcgatacggattcctattatgtctccggttattttgaggaaaacaagctggagcgtatccatatcggtgatccggtggtggtcgatgtgatgggcagcaaggtgcagctcaagggtaaggtggaaggtattgcgggcggcatcgaggatcgcgagcgcagcgattcccaaagcctgctcgccaatgtctcgccgacgttcaactgggtgcgccttgcgcagcgcgtgccggtgcgtgtgaaactggaagatgtgccgcaaggcatccatctggtggcaggccgcacggtgagcgtttcggtggtggat

SEQ ID NO: 189 - DNA, 324 bp, Brucella suis
gtgatcggattaaaatgcttgagtgccattgtctcttactccatggcctctcagagaccggtcgaaacgtcgatgctctggccttcggccaaagtaacgatcgctttcttgacgtcgctctggcgcccgacaaggccgcggaaccgcttaaccttgcctttgcgcactgcagtgttgactgccgtaaccttcacgccgaaaagcgcttcgactgcagccttgatttccggcttcgtcgccttgcgggccacgttgaagacgacctggttgtgttcggaaaccatggtggacttctcggtgatgaccgggctgacgatcacgtcg

SEQ ID NO: 190 - DNA, 123 bp, Brucella suis
atgctctattgcgaaacatccaatgccaatatggcaggcgcagccctcttatatcggggctgcgccttttttcttgccgaatcgataatgccggatttcgtgcagattgcagaagttcgtgga

SEQ ID NO: 191 - DNA, 384 bp, Brucella suis
gtgcgcgaatattatacgataacggaattgacgagggagttcggaatttccacccggacgcttcgtttctatgaggatgaagggttgatcgctccggtgcggcgcggtcgtacacgtctattccgaccgtccgaccgacatcttctcaagcagatcatgcgtggcaagcgattgggcttttctatcgcggaaatccacgaaatcatccagatgtatcgtgagccaccgggggagaccgggcagctcaagctgctgatgaagcgcgtggaagaaaagcgcgccgatttgcgccagaagcgccgggatatcgatgaaacgcttggggaactcgatcaagtcgaggaagcctgcatagagcgtctggcggagcttggcgtatcgacc

SEQ ID NO: 192 - DNA, 1419 bp, Brucella suis
atgtcgaaaccggttattacccgttttgccccctcgccgacgggttatcttcacattggcggagcccgcaccgctctgttcaactggctttacgccaagcattgcggcggcaagatgctgctgcgcatcgaagataccgaccgcgaacgttccacggaggctgcaacggcagccattctggacggcctgacctggcttggcctggattgggatggcgaggcaatctcgcagttcgagcgcgcgccgcgccaccgcgaagtggccgaagagctggttgcaaacggcaaggcctattattgctatgcaagcccggaagagctggaagaaatgcgcgagaaggcacgcgccgaaggccgcccgccgcgttatgacgggcgctggcgcgatcgcgatccctccgaggcaccggcaggcgtaaagccggtcatccgaatcaaggcgcctcgggatggcgaaaccgtcgtgcacgatgccgtgcagggcgatgttcgcttcccgaacaaggatctggacgatttcatcattctccgttcggatggcacaccgacctatatgcacgctgtcgtggtcgatgaccacgacatgggcgtcacccatatcatccgcggcggcgaccatctcaccaacgccgcgcgccagacgatcatctataatgccatgggctgggacgtaccgcagatgtcgcatatctccctcatccatggcgccgatggcgcaaagctttccaagcgtcacggcgcgcttggcgtggatgcctatcgggccatgggctatctgccggccgccctgcgcaactatctcgtccgcctcggctggagccatggcgacgacgaaatcatgtcaaccgaacagatgatcgaatggttcgatgtgaaggacatcaacaagggcgctgcccgctttgatttccagaagctggaggcaatcaacggtctttatatgcgttcaagcgacgatcaggcgctgtttgatgcgctcgttgccgttttgccggaaattgaaggcggcaaggagctggccgaagccctggacgacaaaggccgcgcacaacttctgctcgctatgccggggctgaaggagcgcgcaaagacgcttgttgagcttgccgatggtgcaaagttcatttttgccagccgcccgctcgcgctggacgaaaaagccgcatccttgctcaacgatgaaggccgcgctgtactgaagccggtctatcctgttctggaagcagtgggcgaatggacggctgaatcgcttgatgcggcaatccgcgcccatgcggaagccgaagggctgaaacttggcaagattgcgcaacccttacgcgccgccctgactgggcgcgccacttctccgggtgtattcgacgtattggtggtgcttggaagagaggaatccctcgcccgcatcggcgatcaaatcggt

SEQ ID NO: 193 - DNA, 438 bp, Brucella suis
atgatcgacgatatctacaataaacgcatcctggagtttgcgggcaatatggaacgcatcgggcagcttgccgagccggatgccgttgccacggtgcattcaaaactatgcggctccacggttaccgtttatctgaagatgagggatggtgtggtgacagatttcgcccacgaggtgaaggcatgtgcgctggggcaggcgtcttcctccgtcatggcgcgcaatgtgattggcgccaccgccgacgaattgagggcggcccgcgatgccatgtacaggatgctgaaggaaaacggcccggcaccggaagggcgctttgcggatatgaaatattttgagccggtgcgcgactacaaggcccgccacgcttccacgcttcttaccttcgatgctgtcgctgattgcattcgccagatcgaggaaaaggcgaaagccgcc

SEQ ID NO: 194 - DNA, 369 bp, Brucella suis
atggggtcttgcggcgggaaacatacgggcaagggcgcgcctaaaccttacagccgcaacttcaccgatccatggcgcaagacgccgggacgcctgttcggcacggcactgatacgcttttaccagattacgctctcatcgcttatcggaaactcctgccggcatctgccgacctgttccgaatatgcctatgaagcgattgcgcgccatggtctttggcgcgggggctggatggggttctttcgcgtggtgcgctgcggaccgttcggcacgcatggtttcgaccccgtgccgcgggaactttctcccgatctcaaatggtatatgccctggcgctattggcgctgctcggcttcgcgcaccggaaaa

SEQ ID NO: 195 - DNA, 822 bp, Brucella suis
atgaaatcatcccgcaatattgatcgtttgttggaaatcatggccgcccttcgcaatcccgaaacgggttgcccgtgggatatcgagcagaatttccgcaccatcgcgccctatacgctcgaagaaacctatgaggttctcgacgccattgaacgcgacgatatggatgacctgcgcgaggaacttggcgatcttttgctacaggtcgtcttccatgcccgaatggcggaagaacagggctattttgcctttggtgacgtcgtggaagccatcacgcataagatgatccgccgccacccgcatgttttcggcgatgcgcaggcacgaagcgccggaatggccaagggttcatggaaccgcatcaaggcggaggaaaaggcagagcgcgcggaaaggcgtgcaaaactgggactggaaagcatcgggaaaaccggctttctggatgacattcccaacgccttccctgcccttctgcgcgcgctgaaattacagcagaaagcggcaaaggtcggctttgactggtctgaggcagcccccatcctcgacaagattgccgaggaaacagcggaattgaaagaagccatggccgcaagcgacaaggccgaaatcgaggaagaatatggcgaccttctcttcgccatggtcaatcttggccgtcatcttgaaatcgatgccgaaacggcgctgatttccgcaaacgagaaattcaaacgccgttttcatttcatcgaggaggctttgaaaacgtcaggccgcgatctcgatgcagccagccttgatgaaatggaagaaatctggagcgaagcgaagaaaaaaggcctg

SEQ ID NO: 196 - DNA, 531 bp, Brucella suis
gtgagcactccgccgaaaccattgcgcgcctggcttggcctcggcggcaatatcgatgatccgatcatctccatggcgagcgcgcttcgtatgctggatggacggagcgacacgagggttgtttccgtatccccggtctaccggaccccgccatggggcaagacggatcaggcctggttccacaatgcctgcgcgcaaatcgaaacggccttgcagccactggaactgattgcggcctgtctggacgtggagcgatccttgaaacgtgtgcggctggaacggtggggaccacgcatcatcgatatcgatattcttgttatgcaggatgagacgggcagggcaatcacgatgcaggaaccggcattggagcttccgcatccacgtatgcacgagcgagccttcgttctggtgccgctcaatgatatcgcaccttcgctgcctgtcggtgcgagaactgttgctgaatgggctgccgattgcgaccgtgcggaaatggaaaaagcccgcacggatgcgggctggtggttggaa

SEQ ID NO: 197 - DNA, 834 bp, Brucella suis
atgtcgtcagtcctttcccgctatgcgcttacccgtcgcgcgggcctgaaagcccttctcttcaccgctgcggccctcaccgtcggctttgcatccgctcccagccatgcagaagacaagaccatcaaggtcggcatcatgggcggcgaggacgaagatgtctggaaagtcgttgctgaagaaggcaagaagcatggcctcaatatcgagcgcatcaccttcaacgattataaccagccgaacgaagccctggagcgcaaggaaatcgacgccaacgcattccagcacaagccctatctggacgagcagatcaagcagcacggctacaagatcagcgttgcaggctataccgcagtctggccgatcggcatttattcccgcaaggtgaagaagcttgaggaactgaaggacggcgccaccgttggcgtgccaaacgacccgaccaatgaaggccgtgcgctgcgcgtgctggaagaacagggcctcatcaagctgaagccggatgcgggcattctggcgacgccaatcgacatcgttgaaaacccgaagaagcttgaaatcaaggaactggacgccggcgtcgtcggccgctccatcgatgatcttgacgcggcaatcgtcaacaatgactgggctgcgaaagccgggctgaaaaaagaggatgccattggctgggaatcgaaggaaaacaatccctacaacaatttcatcgccgttcgcaccgatgatctcgaccagccttgggtgaaggcactggttgcctcattccagaacgacgccgtgaaggccgaacttgagcgcgcctacaagggcaccggcatcccggcctggaat

SEQ ID NO: 198 - DNA, 273 bp, Brucella suis
atgacgccggagactgcattgatcaacgagtacctcgccaagcatggtgcacgccggttcgagcagggcgccaccagtggcattcacgggatagcgtcattcatggctgaatatggctacgaggtagccggagcaccgaaaggcggcgtcaaggttcgtcgtggcaaaggccagtggaaacgtatgtccatgccagggctaatcgcaatggcagatgaaattcgccttgcgcaggggcttgagcctttcagcgcagcgcacaaacaggcagca

SEQ ID NO: 199 - DNA, 795 bp, Brucella suis
atgaaaaatgcggcttccattacgatagcgagcggtattatcggcttggttgcgcttggtatcggatattgtgccggtacggcgcgacaggcctccaagcctgtacaggctgcaacctccaccgttgcctttaacccgaaggccgtcgaaaacatcgtgcgcaattatctgcttcagaacccggaactgatgctggaagtgcagaccgcgcttgaaaccaaacaggctcatgcggcacaggaacaggtgaagcaggtactggccgccaatcagagcgttcttttcgaccccaagcatgatgctgtctttggcaacccgaatggcgacgtgacggtctatgaattcttcgattacaattgcggctattgcaagcgcgcgcttcccgatatggaagcgatcctgaaaaaagatccgaatgttcgttatgtcctcaaggagttcccgatcctggggccggattccatgcgtgcgcatgtggtctcccaggctttcaaggcgctgatgccggagaagtacccggaatttcatgaaatgctgcttggcgggcatgggcgtgcaacggaggaatccgcgattgccgacgccgtaaagctcggcgccgatgaagctaagcttcgtgaaaaaatgaaggacccggccatcaccggcgctttccagcggacctaccagcttgcgcaacagctcaacatcaccggcactccgtcctatgtcatcggcgacgaactggtgcccggcgctattggtatcgatggattgcggcagagaatcgcggccgcccgggacgctgcaaagaag

SEQ ID NO: 200 - DNA, 1356 bp, Brucella suis
atgtttcaaaagatactcatagccaatcgtggcgaaatcgctcttcgcgtgctcagggcttgcaaggagctgggcataaagacggtcgccgttcattccacggcagatgccgacgcaatgcatgtgcgcctggcagatgagagcgtatgcatcggaccgccgccctcacgcgacagctatctgaacatccatcagatcgtggccgcctgcgaaatcaccggcgcggatgcaattcaccccggctacggcttcctttcggaaaatgcaaaattcgccgaaatccttgaagcccatgacattaccttcatcggccccactgcttcgcatatccgcatcatgggcgacaagatcgaagccaagcgcaccgcaaagcgcctcggcatcccggtcgttccgggttcggacggcggcgtgacggatgaagtcgaagcagcccgtatcgccaaggaaatcggctatccggtcatcatcaaggcttcggccggcggcggtggtcgtggcatgaaggtcgcccgcagcgcggaagagcttccgatcgcacttgcgacggcacgttctgaagctggcgcggcctttggcgacgatgcggtctacatcgaaaaatatctcgaaaaaccgcgccatatcgaagttcaggtcatgggggacggtgcgggcaatgccgtccatctgggcgaacgcgactgctcgctgcaacgccgtcaccagaaggtctgggaagagggcaactcccccgccctcaatgcggaagcgcgcgaaaagatcggcatgatctgcgccaatgcctgcgcagagcttggctatcgcggcgcaggcacgatcgaatttctctatgaagatggagaattctatttcatcgaaatgaatacgcgccttcaggtggaacatccggttacggaagcgatcaccggcatcgatcttgtgcatgagcagattcgcgttgccgcaggccttggtctttcggtgaagcagaaggatatccgcttttccggccatgccatcgagtgccgtatcaatgcggaagatccgctgaccttcgcaccgtcaccgggcctcatcacgcattatcatacgccgggcgggcttggcgtacgcgtcgattccggcgtttattccggctatcgcattccaccctactatgacagcctgatcggcaagctcatcgtgcatggacgtaaccgcgtcgaatgcatgatgcgtctgcgccgcgcgctggatgagttcgtggtggatggggtaaagaccaccctgcccctgtttcaggatctgatttccaatcaggatatcgccaatggcgattatgacatccactggttggaaaagtacctggccaacaagagcaagaacgaaagcgcc

SEQ ID NO: 201 - DNA, 123 bp, Brucella suis
ttgggttttttcctgaaaagccttgaggatagagcggatgctttgggcctgctgccgctttgtggcttgcagcagggcaacgaatcgtcacatttcatgcttaggaattattccggctttatt

SEQ ID NO: 202 - DNA, 1002 bp, Brucella suis
atgaaaaatctgcattccttgtttcttgcttctgcctttctggctggcttttgcggttcttcccttgcaggcgaacgtgagggcgtggttgtttcgatcaagccgcttcactcgattgtttccgccgtcatgcagggcgtgggtaagccgaagctgatcgttcagggggctgggtccgagcatgtttatagtctgaaaccctccgatgccgaggctatcgagcacgccaaggtgattttctgggccgggccatccatggaaacctttctcgacaagcccattgatacgctgggcgagggcgcgaaagtggtggctcttggcgatgcgaaagggttgacgaaactaaaattccgcgagggtgggccgttcgaggcgcacgatcatggccatggggggagccatgaggaagagcatgacgcacatggttcaggcgatcacgaccatgcggccgaagtcgcagaagaaggccatgaacaccaccatcacggcgaatatgatctgcatttctggcttgacccgcaaaatggcaagattctggctgccgatatcgccaaaacgcttggcgagagcgacccggaacacgccgcgcaatatgaaaagaatgccaaggcctatggagaaaaactcgatgccttgaccagggaagtggctgctgaactgaagccggtcaaggacaagcccttcatcgtctttcatgatgcctatcaatatttcgagaatcgcttcggcatgaaagcagccggctccatcacggtcagcccggaaaaagcgccgggtgcggcgcgtatccagcaaattcacgacaagataaagtcactcggtgcaacctgcgttttctcagagccgcaatttgagccgaaactggtgaagacggtggttgacggaacgaaagcccggacaggtgttcttgatcctcttggcgcagaactgaaggacggccctgatctttatccgcaactcatccgcaatctggccaattcgctgaaagactgcctgtcgaaa

SEQ ID NO: 203 - DNA, 984 bp, Brucella suis
atgtttcgatggggtgtactttccaccgcgaaaatcggcgtcacacaggttattccagcacttgctgcttcggataacggtgtggtccatgcaatagcaagccgcgaccatgcgcgcgcacgcgcggttgccggccgcttcggcgcgccgctcgctttcggttcctatgaggaattgctggccagcaaggaagtggacggtgtctatattccgctgccgacatcccagcatatcgaatggacattgaaggccgccgaagcaggcaagcatgtgctgtgcgaaaagcccattgcgctttgcgcgggcgatatagaccagctcatcgaagcgcgcgacaggcatggcgtcacgattgcggaagccttcatggtctattaccacccgcaatggatcaagctgcgcgcgcttctggccgaaggcgcgattggcaatctgcgtcatgtgcagggtgtgttttcctatttcaatgtcgatcccggcaatatgcgcaaccagccgggccttggcggaggggccttgccggatatcggtgtttacccaaccgtggtgacgcgcatggtgacaggcagggagccgctctcggtgcgcgcttcggtggaatatgatccgggttttggaaccgaccgctacgccaatgtttcggcccgtttcgacgggttcgacctgaccttctatgtcgcgacgcagcttgccggccgccaatgcatggttttccatggcgataagggcttcatcgaggttcatgcgccattcaataccggcaaatatggccatggccgcatcaccctgcacgatgcgagccatatgcaggccaccgaatggtcgtttggcgatgcaaaccactatcagcttcaggcgcagagctttgttcgtgcggcgcggggtgaaaatgtgccgctcttctcgctggaaaattccagggcgaaccagaaattcatcgatgcgatttattccgcgggcagaagcggggtgagcgagactgtg

SEQ ID NO: 204 - DNA, 1983 bp, Brucella suis
atgaagttcacgagaacgctggtgctcgccagcacatttctgcttgccaccgtcgccacatcgcaggctcaggaagtaaagcgtgatacgaaaaagcggggtgaagttgtccttaaacccattacgatcataagccacggcaaggacaatatcgaagcgaccggcggcactgtccttacttataaagatattgaaaagctgcaacctgccaacgtttccgagttgttttcgcgccagtcctccatcgctgtttccggcggtggcggcccgtcgaagcgtattcacattcttggcatggagcaatccaatctggcggtcagtgtggatggcgtgccgcaaacggcgacaagctggcaccatacgggttccaacgtaatcgatcctgcgtttctcaagcgcgtggaagtggaggccggagcggccgccgcagattccggctttggcgcggccgctggtgcgatccgctacgagaccgttaatgcgctcgatctgctggagccgggcaaaaccttcggtgcgcgcatcataggttcctacggtacaaatggcaggggtttttccggcagtaccgccgcctatggcctcaaggatggcttcgactggctgctgatgctgcatggcacgtcgggccacaattataaaaacggcgatggaaccgaaatactcggcaccgagcctgccgcgcgcaatattctgggcaaggccggttatgaattcgacggcaaccgcatcgatattggctatgaacgcagccgcgacaaggccgaccgtcttatcaagatgaatatggggctgcccggcgacaccgaatatccgcttgaggttgcgcgtgattccgtgaacataaaatatacccgtaccgatgctacggacatgtgggatccggaggtgcagttctattacaatcgcaacgattattggcgcaacgattatcagaacaggacaaacggcaacatgatcctcaaggaggatctgtatggcggcaagttgcagaatactttcacgatcgactacggcaagattaccgccggtatcgattttggcaagcacgactacaatacggacaattacggtcataatgaccgccgctaccgcaagttcaacacccagcaggtcggtgcattcacgcaaggccggtttgaattcgataacggcttcagcctttcgacaggtgcccgctatgactatagccggtttgcggactggaacgatgaggtgttttcagatagcggcgccagcgtgaacggcacgctatcatacaagttcaacgagcatatcgaggtttttgccggtgcttcacggacctggcttggctacgtcctcggtgattacggctatgtccacgcccgcaataatgctttctataccgacccgaccttcagccccggaagggcgcgcaattataaagcgggtgtgaatttcggcggtgcagactggagtgccggcattacgcttttcgacacgcgcatcgccggtctgccgaattatgacagccagaagcttggcaacgatccggaagaatatcgttcgcgaggctttaccctcaatgcgcgctatatctggaactacacgaccattggcgcaacctttaccaaggccaaggttacggcgggcgacgacccagtcttgccgaacagcggcagcttcatgccgatcggtgatatggcgacgcttttcatcgaccaggaaatccccgactacaatatgaaagtgggtgctacgctggcctgggcgggtagaatttcagacgaggctgctactgctgccaacttctacgatcagcctgcctatacggttgtcaacgcctatgcggaatggaatccgccagcggttaagaatatgacgttgcgcgtgggtgtcgagaacctgttcaacgaaaactattacgagagaaccagcttcgcgccgagtcagaagcgtggtggcatcgatccggtttgggcgccgggccggaccttcactttccagacggccttcaagttc

SEQ ID NO: 205 - DNA, 1326 bp, Brucella suis
atgagcgagatcggttcttatcacgatacgatttttgcattatcgagcggacgcttgccctcaggggtcgcggtcattcgcatttccggcccaaaaactcgattcgtttacgaaacaatctgccaagcaattccggaaccgcgccatgccgcattgcttacattccgtagccgaaatggtgacgccattgatcgcggcctcactctgtttttccctgctcctcatagttttactggggaagactgcgccgaatttcatttgcacggcggtaaggccgttgtggaaaagatgcttgcggtgttgggcgaactgccgggatgccgaattgcggaggctggtgaatttacgcgacgtgcttttgccaatggcaagatggacttgacgattgcagaaggtctggccgacctcattgctgcggaaaccgaggggcagcgacggcttgcaatgcaagtggcttccggtaatcagcgcaaactatatagtgagtggcgccagcgacttatcaatgcgcgggctttcattgaagctgaactggattttgccgatgaaagcgacgtacccggttctgtttccatgcaggtctggcaacagctttcggcgctcaaacatgaaatcgagcaccatatcgcaagcggtaagcgcgctgcgatgctgcgggatggattgcatgttgttattgtcggcgcgccaaatgccggtaaatccagcttgcttaattttctagccgggcgcgatgttgcgatcatttccaaagaagctggtacgacgcgcgatcttctggaagtgaaactcgatctcggcggtatcccggtttatgtcaccgatacggcgggtcttcgcgaaacggatagtgtcgttgagaagatcggcattgagcgtgcgcgtgcccggatggccgaagccgatctggtgctctcgctggaagatatgagcggacctgtatctgttaccgttgagaaaatcgaagcagaaacctggctgatcggtaccaaggccgacctcggtggaagtgcttcgggcttgtggaaatatcacatttccaccatgactggcagtggtctggaacaattgctggacgctctacaggcttttgctgaagcgaagatcggccagattgaagatgctgttcctacccggcagcgccatatcaacctgttgcgggcaacgattgaagaaatcgaaaaagcaattgagggtgacgatcttccattggagttaagagccgagaatatgcggcttgcttcgcaattccttggacggatcacgggcgatgtggatgtagaggaaattctggacgttattttctcgcaattctgcatcgggaag

SEQ ID NO: 206 - DNA, 54 bp, Artificial Sequence
  Description of Artificial Sequence Synthetic primer
caccatgagcgaaccgaacccaaaacgcggtttttcgagccgccctttttgccc

SEQ ID NO: 207 - DNA, 54 bp, Artificial Sequence
  Description of Artificial Sequence Synthetic primer
caccgtgaaggggatttcgcttccacactgccttgttgcgcgaatcccggatat

SEQ ID NO: 208 - DNA, 54 bp, Artificial Sequence
  Description of Artificial Sequence Synthetic primer
caccatgaaactgtcggcaagagcttcaaccgcctgccgccgagacgcatgcgg

SEQ ID NO: 209 - DNA, 54 bp, Artificial Sequence
  Description of Artificial Sequence Synthetic primer
caccatgccgaaggatgccaattcagcctatcttcctgcggtgccggggtaggc

SEQ ID NO: 210 - DNA, 54 bp, Artificial Sequence
  Description of Artificial Sequence Synthetic primer
caccgtgtgtcgagggcggattggccttcgacgaatttcaaagcgatgatttcg

SEQ ID NO: 211 - DNA, 54 bp, Artificial Sequence
  Description of Artificial Sequence Synthetic primer
caccgtgaagtggttcaacaccaccaaggtgctgcgacgagattgtcggcagac

SEQ ID NO: 212 - DNA, 54 bp, Artificial Sequence
  Description of Artificial Sequence Synthetic primer
caccgtgggggccttccgccaggatgagctcgttcttggaggatgaatgaatga

SEQ ID NO: 213 - DNA, 54 bp, Artificial Sequence
  Description of Artificial Sequence Synthetic primer
caccatgtatcgttatgatgaattcgaccggcagcacttcttatctccccataa

SEQ ID NO: 214 - DNA, 54 bp, Artificial Sequence
  Description of Artificial Sequence Synthetic primer
caccttgcttccagcgccagcaggcgtctggctgcgacgggaacaggtttcagg

SEQ ID NO: 215 - DNA, 54 bp, Artificial Sequence
  Description of Artificial Sequence Synthetic primer
caccatgacgatgcaatggttctcgctcgatccagtgtgcgctggaagcgcaac

SEQ ID NO: 216 - DNA, 54 bp, Artificial Sequence
  Description of Artificial Sequence Synthetic primer
caccatgtctcccaaaaagctggaaatgcaagactgatatggcttgtgaaaagc

SEQ ID NO: 217 - DNA, 54 bp, Artificial Sequence
  Description of Artificial Sequence Synthetic primer
caccatggatcttagcgatctgattcagcggcggcgttggacgtcgggtgagcc

SEQ ID NO: 218 - DNA, 54 bp, Artificial Sequence
  Description of Artificial Sequence Synthetic primer
caccatgtatatggaacgcgagacgggcagttccgcttgggcgcagcaccgccc

SEQ ID NO: 219 - DNA, 54 bp, Artificial Sequence
  Description of Artificial Sequence Synthetic primer
caccttggtcagaatgcgggggcaggcaaggccgtcgaccataccgtgtgcacg

SEQ ID NO: 220 - DNA, 54 bp, Artificial Sequence
  Description of Artificial Sequence Synthetic primer
caccatgcacaagccgaaactgcttctgcttgcatattgtccttcgccccggcc

SEQ ID NO: 221 - DNA, 54 bp, Artificial Sequence
  Description of Artificial Sequence Synthetic primer
caccatgactgaaacaggtttcagtttctaaccgcgcccaattcgtgcttcgtg

SEQ ID NO: 222 - DNA, 54 bp, Artificial Sequence
  Description of Artificial Sequence Synthetic primer
caccttgccgtttcactggcaggcccggtagtccggcgacaagagcctcgcgct

SEQ ID NO: 223 - DNA, 54 bp, Artificial Sequence
  Description of Artificial Sequence Synthetic primer
caccatgaatatccaggccacttcggcacagcatgaacggcgagccagttggaa

SEQ ID NO: 224 - DNA, 54 bp, Artificial Sequence
  Description of Artificial Sequence Synthetic primer
caccatgcatgaggaaaagctgcccacgcttcggctgcgataagcgacactgcg

SEQ ID NO: 225 - DNA, 54 bp, Artificial Sequence
  Description of Artificial Sequence Synthetic primer
caccatgaaatcctgtctgttgagcctgaataaaagcttgccgggaaatatctc

SEQ ID NO: 226 - DNA, 54 bp, Artificial Sequence
  Description of Artificial Sequence Synthetic primer
caccttgtcgatcctgcgctttgctgccgatgcagccgggctgcgcgcccggct

SEQ ID NO: 227 - DNA, 54 bp, Artificial Sequence
  Description of Artificial Sequence Synthetic primer
caccatgaaatatccgcgcaatctcgtcgatagagtgtttcggggaggatgtct

SEQ ID NO: 228 - DNA, 54 bp, Artificial Sequence
  Description of Artificial Sequence Synthetic primer
caccatgctggagaaaagcctccagcaatggcgctgccgtgcgcgaccacatcg

SEQ ID NO: 229 - DNA, 54 bp, Artificial Sequence
  Description of Artificial Sequence Synthetic primer
caccatgcgctcgtgcggggcagggcaaggactttcactgaagcaacggtcgct

SEQ ID NO: 230 - DNA, 54 bp, Artificial Sequence
  Description of Artificial Sequence Synthetic primer
caccgtgctggcccgggccatattcacgagtcctcggaaacgcagggcagagga

SEQ ID NO: 231 - DNA, 54 bp, Artificial Sequence
  Description of Artificial Sequence Synthetic primer
caccgtgaaattcaatcgtccgcacgcgcatcggtgaaactattttcatcgcgc

SEQ ID NO: 232 - DNA, 54 bp, Artificial Sequence
  Description of Artificial Sequence Synthetic primer
caccatgtgctcaaagggcaagaagcttctgcagcactcgacgacttgctcttc

SEQ ID NO: 233 - DNA, 54 bp, Artificial Sequence
  Description of Artificial Sequence Synthetic primer
caccatgtcggaactgctttttgaaaccggatttgagagaggtcgggttcaaga

SEQ ID NO: 234 - DNA, 54 bp, Artificial Sequence
  Description of Artificial Sequence Synthetic primer
caccatgtttcaaaagttttcagcggcggtttcaccgtttcctgttttttctgg

SEQ ID NO: 235 - DNA, 54 bp, Artificial Sequence
  Description of Artificial Sequence Synthetic primer
caccatgcgaaggcgcggcggtgtggttgcggcggcaatcggcgagcgcctgcg

SEQ ID NO: 236 - DNA, 54 bp, Artificial Sequence
  Description of Artificial Sequence Synthetic primer
caccgtgcaattatgcgacccggcgcatatggcaaacggcggcgaagatcgctc

SEQ ID NO: 237 - DNA, 54 bp, Artificial Sequence
  Description of Artificial Sequence Synthetic primer
caccttgaaagaaaccatcatgtcgatcaggccccaaagcggcgcttttcgcca

SEQ ID NO: 238 - DNA, 54 bp, Artificial Sequence
  Description of Artificial Sequence Synthetic primer
caccatgcgagttcttcatttttttaagactgcatcagcgacgtataatttttc

SEQ ID NO: 239 - DNA, 54 bp, Artificial Sequence
  Description of Artificial Sequence Synthetic primer
caccatgcgcctgcaccggcaacgcacgcaaaaatcccgccataaattttgtga

SEQ ID NO: 240 - DNA, 54 bp, Artificial Sequence
  Description of Artificial Sequence Synthetic primer
caccttgccctatcgcacaggcttcgctaatcggttgaagcgaaggtgttgtca

SEQ ID NO: 241 - DNA, 54 bp, Artificial Sequence
  Description of Artificial Sequence Synthetic primer
caccatgcgtaatacggcagaaaaagatcgagtaccgaaacttttgtgcgctcg

SEQ ID NO: 242 - DNA, 54 bp, Artificial Sequence
  Description of Artificial Sequence Synthetic primer
caccatgacggatcagggggagaacgagagccgccgttgcgggtgctgcccact

SEQ ID NO: 243 - DNA, 54 bp, Artificial Sequence
  Description of Artificial Sequence Synthetic primer
caccatggaaaaaagccgtaaatcgcatgcttcccctgcataagtttggaaatg

SEQ ID NO: 244 - DNA, 54 bp, Artificial Sequence
  Description of Artificial Sequence Synthetic primer
caccatgagccggttttccgcactcatgcgccaacctcgcgatgcggcagcgga

SEQ ID NO: 245 - DNA, 54 bp, Artificial Sequence
  Description of Artificial Sequence Synthetic primer
caccatgaaaatctgtacctctattctcggtccagttccagccggccggaaact

SEQ ID NO: 246 - DNA, 54 bp, Artificial Sequence
  Description of Artificial Sequence Synthetic primer
caccatggctgaaatattttcagccggagttgcgcctgcggggcattaaccttg

SEQ ID NO: 247 - DNA, 54 bp, Artificial Sequence
  Description of Artificial Sequence Synthetic primer
caccgtgtcttcctgtcgcgattcggacagtttttagcgccgacagcctttggc

SEQ ID NO: 248 - DNA, 54 bp, Artificial Sequence
  Description of Artificial Sequence Synthetic primer
caccatgaagagaatccttctagctgaagggcggcgatcagcattttttcgatt

SEQ ID NO: 249 - DNA, 54 bp, Artificial Sequence
  Description of Artificial Sequence Synthetic primer
caccatggctgatcaggctatatccgataggcctgcgcgcggttgcgcctgtgc

SEQ ID NO: 250 - DNA, 54 bp, Artificial Sequence
  Description of Artificial Sequence Synthetic primer
caccttgcaggataaaacagctctcatcggccgtagaaatcattgcgtcctacc

SEQ ID NO: 251 - DNA, 54 bp, Artificial Sequence
  Description of Artificial Sequence Synthetic primer
caccttgtgtcattcgcaatcgatccgtcaaaccggtggattgaataatctgtg

SEQ ID NO: 252 - DNA, 54 bp, Artificial Sequence
  Description of Artificial Sequence Synthetic primer
caccatgtccacaatccacttcgataaaattccgctgggtcgccttccggcgct

SEQ ID NO: 253 - DNA, 54 bp, Artificial Sequence
  Description of Artificial Sequence Synthetic primer
caccgtggatgccataatgatggagcataggctgcattgtttgcctgccggctt

SEQ ID NO: 254 - DNA, 54 bp, Artificial Sequence
  Description of Artificial Sequence Synthetic primer
caccatgaaaaagctgcttattttggccgcagtttgcgcagttgcgaaacttcc

SEQ ID NO: 255 - DNA, 54 bp, Artificial Sequence
  Description of Artificial Sequence Synthetic primer
caccttgggattgctggaaacggccattaagccgctgaccgaacgcatgaaagg

SEQ ID NO: 256 - DNA, 54 bp, Artificial Sequence
  Description of Artificial Sequence Synthetic primer
caccgtgctgacagaagcgatttcatgggtttgccgggcgggtcggggggaaac

SEQ ID NO: 257 - DNA, 54 bp, Artificial Sequence
  Description of Artificial Sequence Synthetic primer
caccatgcgtagtcgcagtttttcatgttgtaactgccttttacagcgtaatca

SEQ ID NO: 258 - DNA, 54 bp, Artificial Sequence
  Description of Artificial Sequence Synthetic primer
caccatgctggcgcgtgaacagatgcagcgcgcgccctgctgtgtgacctgcaa

SEQ ID NO: 259 - DNA, 54 bp, Artificial Sequence
  Description of Artificial Sequence Synthetic primer
caccatgactgtcgcagaagcggttgccctttcttgtctttcagcgttatgaag

SEQ ID NO: 260 - DNA, 54 bp, Artificial Sequence
  Description of Artificial Sequence Synthetic primer
caccgtgatgaagtccttatttattgcatttcgatcacgccgcaggcaaaacgg

SEQ ID NO: 261 - DNA, 54 bp, Artificial Sequence
  Description of Artificial Sequence Synthetic primer
caccatgggaccgatcacggataaggtggttcccagttttcaatgcggattttt

SEQ ID NO: 262 - DNA, 54 bp, Artificial Sequence
  Description of Artificial Sequence Synthetic primer
caccgtggcaattgcgcaaaactggctctatgccgtgccggaatgctgccggca

SEQ ID NO: 263 - DNA, 54 bp, Artificial Sequence
  Description of Artificial Sequence Synthetic primer
caccttgcgacgtgtgggacaaggcgcgcgtttctttctttgatctgggcttca

SEQ ID NO: 264 - DNA, 54 bp, Artificial Sequence
  Description of Artificial Sequence Synthetic primer
caccatgactgcccattcccgacaggacctaggccagcgactttcttcaggtcg

SEQ ID NO: 265 - DNA, 54 bp, Artificial Sequence
  Description of Artificial Sequence Synthetic primer
caccttgcaaattttcgtcttcggacgtttgcggttttctcctgacggacggat

SEQ ID NO: 266 - DNA, 54 bp, Artificial Sequence
  Description of Artificial Sequence Synthetic primer
caccatgaccacattgaccgcgaaatggaannnnnatgcacgcccgcgcatacc

SEQ ID NO: 267 - DNA, 54 bp, Artificial Sequence
  Description of Artificial Sequence Synthetic primer
caccttgaactggctgacggattacaagatttctcacgtccggcgcggaagata

SEQ ID NO: 268 - DNA, 54 bp, Artificial Sequence
  Description of Artificial Sequence Synthetic primer
caccttgcgacgtcaggccggtgctcgttatgcctttgctgtctgggaggacat

SEQ ID NO: 269 - DNA, 54 bp, Artificial Sequence
  Description of Artificial Sequence Synthetic primer
caccatgaataagctcggcgtgtttatcggtctgcaacagatttaagcagagac

SEQ ID NO: 270 - DNA, 54 bp, Artificial Sequence
  Description of Artificial Sequence Synthetic primer
caccatgttgatcgtcggtgccggtatatgaaattgccgtcccgcaaggggccg

SEQ ID NO: 271 - DNA, 54 bp, Artificial Sequence
  Description of Artificial Sequence Synthetic primer
caccatgcagcgccttggcaacggcagcgcgcgatgaacaaaagggagggcccg

SEQ ID NO: 272 - DNA, 54 bp, Artificial Sequence
  Description of Artificial Sequence Synthetic primer
caccgtgtgttgtatgaattttataaaatctgacttgacgtgatatgaaatgcc

SEQ ID NO: 273 - DNA, 54 bp, Artificial Sequence
  Description of Artificial Sequence Synthetic primer
caccatgtccgccgaaatcatttcccaaagcgggccggataacgttccttcaaa

SEQ ID NO: 274 - DNA, 54 bp, Artificial Sequence
  Description of Artificial Sequence Synthetic primer
caccatgaaatcacggcgcatgttccgtcaagggttgaaatcgtccagcttacg

SEQ ID NO: 275 - DNA, 54 bp, Artificial Sequence
  Description of Artificial Sequence Synthetic primer
caccatgagtatcaacgcgaccacgctcgcggcctgcggatcaatgttggcagg

SEQ ID NO: 276 - DNA, 54 bp, Artificial Sequence
  Description of Artificial Sequence Synthetic primer
caccgtggccgaaaccaatacctcgccttgtcgatccccaccatgccggtcaga

SEQ ID NO: 277 - DNA, 54 bp, Artificial Sequence
  Description of Artificial Sequence Synthetic primer
caccttgccggaccgattatgccggacaatcgttcgccagctatatgagcggac

SEQ ID NO: 278 - DNA, 54 bp, Artificial Sequence
  Description of Artificial Sequence Synthetic primer
caccatgcgcccgcgacggcgtgataacgattaannnnnatgagggcattgacc

SEQ ID NO: 279 - DNA, 54 bp, Artificial Sequence
  Description of Artificial Sequence Synthetic primer
caccatgtccagagccaggatttccaactttcctgattgatcggcagcgccacg

SEQ ID NO: 280 - DNA, 54 bp, Artificial Sequence
  Description of Artificial Sequence Synthetic primer
caccatgcgttgcatagatgcaacgctataatcacctgtccagctgtgtgcttt

SEQ ID NO: 281 - DNA, 54 bp, Artificial Sequence
  Description of Artificial Sequence Synthetic primer
caccatgacgatgtttcttgtgacgccactagggaaatccgccgccagctatcg

SEQ ID NO: 282 - DNA, 54 bp, Artificial Sequence
  Description of Artificial Sequence Synthetic primer
caccgtggaggatggcagcatgacgaggccacactccgcggtcgaatctcaggg

SEQ ID NO: 283 - DNA, 54 bp, Artificial Sequence
  Description of Artificial Sequence Synthetic primer
caccttgcaggaggctctggccctgttttgcaggcaaaaaggtcgttgaaaagt

SEQ ID NO: 284 - DNA, 54 bp, Artificial Sequence
  Description of Artificial Sequence Synthetic primer
caccatgtcaaatctgcaaacccgcatcagaaactaaagaagattgcagacgga

SEQ ID NO: 285 - DNA, 54 bp, Artificial Sequence
  Description of Artificial Sequence Synthetic primer
caccatgacgaagcgagtgccccttacgaagcagttccgaggcatcgcaacccg

SEQ ID NO: 286 - DNA, 54 bp, Artificial Sequence
  Description of Artificial Sequence Synthetic primer
caccgtggtagtcatggcccaatccttgaacctttaaccgcgccgaaagtcagg

SEQ ID NO: 287 - DNA, 54 bp, Artificial Sequence
  Description of Artificial Sequence Synthetic primer
caccttgccatggcgtgcagccatccacgataaggaggagacgaatgcaggatt

SEQ ID NO: 288 - DNA, 54 bp, Artificial Sequence
  Description of Artificial Sequence Synthetic primer
caccatgagggagccgaccttggaaactgtcctgtcacgcctacatccgcgccc

SEQ ID NO: 289 - DNA, 54 bp, Artificial Sequence
  Description of Artificial Sequence Synthetic primer
caccatgatgactgacatgcgcattcagatgcattcaacttcttaccttcggga

SEQ ID NO: 290 - DNA, 54 bp, Artificial Sequence
  Description of Artificial Sequence Synthetic primer
caccttgcgttctcttgcccagcggatcggtaaattgaacgtggttgtagagta

SEQ ID NO: 291 - DNA, 54 bp, Artificial Sequence
  Description of Artificial Sequence Synthetic primer
caccatggtaacctatatcgaagcgacaaccgcatggaaggcgggccaaagata

SEQ ID NO: 292 - DNA, 54 bp, Artificial Sequence
  Description of Artificial Sequence Synthetic primer
caccatgtacggacgcgcccgttccggagcttctttttctttgccgcggccttt

SEQ ID NO: 293 - DNA, 54 bp, Artificial Sequence
  Description of Artificial Sequence Synthetic primer
caccttgaccggccatatcaagattttcgtgagccgataatatggcgctttttc

SEQ ID NO: 294 - DNA, 54 bp, Artificial Sequence
  Description of Artificial Sequence Synthetic primer
caccatgttgcgctatgtcggcaactgcctgcaacttgcggtaaaaacaagcaa

SEQ ID NO: 295 - DNA, 54 bp, Artificial Sequence
  Description of Artificial Sequence Synthetic primer
caccatgacgactgccaacgatattctgataccgagtmatacatatcgaactcg

SEQ ID NO: 296 - DNA, 54 bp, Artificial Sequence
  Description of Artificial Sequence Synthetic primer
caccgtgaagattccggcggttggactgcatcaaaccctgtcaatagacgcgcg

SEQ ID NO: 297 - DNA, 54 bp, Artificial Sequence
  Description of Artificial Sequence Synthetic primer
caccatggccgcaacattcgatacgatttgcgacggggcagcgcttcgaggaac

SEQ ID NO: 298 - DNA, 54 bp, Artificial Sequence
  Description of Artificial Sequence Synthetic primer
caccatgggatgtggtagggtaatcgcggttcggcagcggccagacctgccatg

SEQ ID NO: 299 - DNA, 54 bp, Artificial Sequence
  Description of Artificial Sequence Synthetic primer
caccatggcaattgcaccaaaggcgggttgtcgatacggatcgtcacgaaacgc

SEQ ID NO: 300 - DNA, 54 bp, Artificial Sequence
  Description of Artificial Sequence Synthetic primer
caccatgtgcttgggaaagggggctggctgtctgagaaggagatcataacatgg

SEQ ID NO: 301 - DNA, 54 bp, Artificial Sequence
  Description of Artificial Sequence Synthetic primer
caccatgagtattcttggtggtcatggagaaaactctctctcagccattggccg

SEQ ID NO: 302 - DNA, 54 bp, Artificial Sequence
  Description of Artificial Sequence Synthetic primer
caccatggcggcagacgcgccacatgatattttaacagaatcgccggaaccgct

SEQ ID NO: 303 - DNA, 54 bp, Artificial Sequence
  Description of Artificial Sequence Synthetic primer
caccatgcggctgagattgttttcggcaaatcgtattcgtttgaaaccattgct

SEQ ID NO: 304 - DNA, 54 bp, Artificial Sequence
  Description of Artificial Sequence Synthetic primer
caccgtggcgcgtccgcccaatcttgcgacgcttcccggaaacgataaccgacg

SEQ ID NO: 305 - DNA, 54 bp, Artificial Sequence
  Description of Artificial Sequence Synthetic primer
caccatgttcttcgcggtgctgggcgctgtaaccggcctccggcattgttgcgg

SEQ ID NO: 306 - DNA, 54 bp, Artificial Sequence
  Description of Artificial Sequence Synthetic primer
caccatgggaaacgcccgacattgcaggatttgtcccggaagtggtcgtggatc

SEQ ID NO: 307 - DNA, 54 bp, Artificial Sequence
  Description of Artificial Sequence Synthetic primer
caccgtgactttatcagggcaagttccggtcttcctgcgaaaacctgctcataa

SEQ ID NO: 308 - DNA, 54 bp, Artificial Sequence
  Description of Artificial Sequence Synthetic primer
caccatggccgcgcattatgcaggcattcgccgcgcggcagaagctcgaccagc

SEQ ID NO: 309 - DNA, 54 bp, Artificial Sequence
  Description of Artificial Sequence Synthetic primer
caccatgcccaaggggctttatgcgcgctagccggaatatgaatggtggcgcgc

SEQ ID NO: 310 - DNA, 54 bp, Artificial Sequence
  Description of Artificial Sequence Synthetic primer
caccatgttctcaggacatggcatggccgaccgatacgggaatacacgtgcagg

SEQ ID NO: 311 - DNA, 54 bp, Artificial Sequence
  Description of Artificial Sequence Synthetic primer
caccttgctctggatcacgctcgtcagcgttttgtgtaaaacaaaaaagggtgc

SEQ ID NO: 312 - DNA, 54 bp, Artificial Sequence
  Description of Artificial Sequence Synthetic primer
caccgtgaagcgtatcaggaaccggaaacaaagcccccgtagaggctgacctga

SEQ ID NO: 313 - DNA, 54 bp, Artificial Sequence
  Description of Artificial Sequence Synthetic primer
caccatgtcgaaagcacgcacttcacccgttttaccgctccagccgtaatgcca

SEQ ID NO: 314 - DNA, 54 bp, Artificial Sequence
  Description of Artificial Sequence Synthetic primer
caccttgcttggctttctgtttgatggcgaaacaggaaccgctcttctagggga

SEQ ID NO: 315 - DNA, 54 bp, Artificial Sequence
  Description of Artificial Sequence Synthetic primer
caccatgagcccccacgacagcaacatcacaaacccaaatatgctttttgcatc

SEQ ID NO: 316 - DNA, 54 bp, Artificial Sequence
  Description of Artificial Sequence Synthetic primer
caccgtggagcgaccctaccagtcgcgcacggcgcgacgatcggctgtgccttc

SEQ ID NO: 317 - DNA, 54 bp, Artificial Sequence
  Description of Artificial Sequence Synthetic primer
caccatgttcagccgattcttccgtaatgggcgctgccgatcaatatgcccgcc

SEQ ID NO: 318 - DNA, 54 bp, Artificial Sequence
  Description of Artificial Sequence Synthetic primer
caccttgcagggtaaaactgacgtgatatgaaggagacgggacgcttctgcttg

SEQ ID NO: 319 - DNA, 54 bp, Artificial Sequence
  Description of Artificial Sequence Synthetic primer
caccgtgaacgaggctgatgcgcttgataaaaacccttttcgatgcgggaatag

SEQ ID NO: 320 - DNA, 54 bp, Artificial Sequence
  Description of Artificial Sequence Synthetic primer
caccttgcgcaagccgtacagtccatcgatagcttttccgcacccgtcaaaagc

SEQ ID NO: 321 - DNA, 54 bp, Artificial Sequence
  Description of Artificial Sequence Synthetic primer
caccgtggggccgcttgcaaccggctttttcctcgtcgggcgatcaatatgccg

SEQ ID NO: 322 - DNA, 54 bp, Artificial Sequence
  Description of Artificial Sequence Synthetic primer
caccttgctggcttacatatccgggcggttcggctgcctcccgtcgaaaggcgt

SEQ ID NO: 323 - DNA, 54 bp, Artificial Sequence
  Description of Artificial Sequence Synthetic primer
caccttgatgtccgaatatcttttggaaaggccgcgttggttgcaggcttgcca

SEQ ID NO: 324 - DNA, 54 bp, Artificial Sequence
  Description of Artificial Sequence Synthetic primer
caccttgtctcctgcgctttcttctgccctcgcaaagttgccttttccttctca

SEQ ID NO: 325 - DNA, 54 bp, Artificial Sequence
  Description of Artificial Sequence Synthetic primer
caccatggcaggagcgggcatgaaacccattgaccgtgtgaagattgctggtct

SEQ ID NO: 326 - DNA, 54 bp, Artificial Sequence
  Description of Artificial Sequence Synthetic primer
caccatggcgctggaaccggctgctgctggattacgtcgcggatgcgctccacg

SEQ ID NO: 327 - DNA, 54 bp, Artificial Sequence
  Description of Artificial Sequence Synthetic primer
caccatgaaactgatggaatccgcgcctttcctttctgcgcgcgcgtggtcatg

SEQ ID NO: 328 - DNA, 54 bp, Artificial Sequence
  Description of Artificial Sequence Synthetic primer
caccgtgaaggcgatttttcaaacggcgcctgcatccccacctgaatacgacca

SEQ ID NO: 329 - DNA, 54 bp, Artificial Sequence
  Description of Artificial Sequence Synthetic primer
caccatgccaattctttacacgacccagtggctacgctcaggcgaacgtcgaga

SEQ ID NO: 330 - DNA, 54 bp, Artificial Sequence
  Description of Artificial Sequence Synthetic primer
caccatgtccgtcgtcctgccgccggtcaccctaccacaaaacaggcaagccgc

SEQ ID NO: 331 - DNA, 54 bp, Artificial Sequence
  Description of Artificial Sequence Synthetic primer
caccatggctgtccgccgcatcgttgccaatgcttgtgggtcagaatattcacg

SEQ ID NO: 332 - DNA, 54 bp, Artificial Sequence
  Description of Artificial Sequence Synthetic primer
caccatgacgcaaaacgaacgtataggcgacccgataccaaaaccctgctgatg

SEQ ID NO: 333 - DNA, 54 bp, Artificial Sequence
  Description of Artificial Sequence Synthetic primer
caccatgctgattgtcgagtcgcatcgcggttcttgtccgggttacgatattgc

SEQ ID NO: 334 - DNA, 54 bp, Artificial Sequence
  Description of Artificial Sequence Synthetic primer
caccatggcaacaccgttccgcaattgcaatccttgcggaacagaatgcgcccg

SEQ ID NO: 335 - DNA, 54 bp, Artificial Sequence
  Description of Artificial Sequence Synthetic primer
caccttgccgattgtgctggcaagcacgaaaatgcgtcgcgtttttccatattc

SEQ ID NO: 336 - DNA, 54 bp, Artificial Sequence
  Description of Artificial Sequence Synthetic primer
caccatgacaggagaggacatgcaagcgagcggcgcatgcgccagcggcggccc

SEQ ID NO: 337 - DNA, 54 bp, Artificial Sequence
  Description of Artificial Sequence Synthetic primer
caccatggcgcggctcttagcacagaatattccctgaagggcatttccgcggca

SEQ ID NO: 338 - DNA, 54 bp, Artificial Sequence
  Description of Artificial Sequence Synthetic primer
caccttgccaattgagacgatcttgcgtttcgatatcctgcccgtcgccgcacg

SEQ ID NO: 339 - DNA, 54 bp, Artificial Sequence
  Description of Artificial Sequence Synthetic primer
caccgtgctcgaaaaccgatatgctcatcagttcgatatcgtattgcgcggacg

SEQ ID NO: 340 - DNA, 54 bp, Artificial Sequence
  Description of Artificial Sequence Synthetic primer
caccatgcgctcctccggcagatcgaaatccccgatctcccggcggaattttcc

SEQ ID NO: 341 - DNA, 54 bp, Artificial Sequence
  Description of Artificial Sequence Synthetic primer
caccatggcgcaaaccatcggcctttttctccatgatggtcgcaatggctgcct

SEQ ID NO: 342 - DNA, 54 bp, Artificial Sequence
  Description of Artificial Sequence Synthetic primer
caccatgaccgcaagccaagattttgttgatacctgtagtgttccgacttgaac

SEQ ID NO: 343 - DNA, 54 bp, Artificial Sequence
  Description of Artificial Sequence Synthetic primer
caccgtgcgccccgagcatgtccgctcacgaaatcatcatcgcccgtgggcacc

SEQ ID NO: 344 - DNA, 54 bp, Artificial Sequence
  Description of Artificial Sequence Synthetic primer
caccatgaacttcaagacccgtatggctaaagtggcttgaagcagactttggtg

SEQ ID NO: 345 - DNA, 54 bp, Artificial Sequence
  Description of Artificial Sequence Synthetic primer
caccatgcgcgttcttggaatagaaacaatgcctttgctccccgccgtcccgtg

SEQ ID NO: 346 - DNA, 54 bp, Artificial Sequence
  Description of Artificial Sequence Synthetic primer
caccatgcagactgtgctcagcattgctgtttgcgggccagccttcctgcaagg

SEQ ID NO: 347 - DNA, 54 bp, Artificial Sequence
  Description of Artificial Sequence Synthetic primer
caccatgctactgccacccatcaaagaaatattggcgcatgttcagcaaaagtg

SEQ ID NO: 348 - DNA, 54 bp, Artificial Sequence
  Description of Artificial Sequence Synthetic primer
caccatggcaaattattccgttacttccgaagatgctggttgaagccgccaggc

SEQ ID NO: 349 - DNA, 54 bp, Artificial Sequence
  Description of Artificial Sequence Synthetic primer
caccatgaacaagatgtccaggatcataaggcagcctgtaattgccgctcgcgc

SEQ ID NO: 350 - DNA, 54 bp, Artificial Sequence
  Description of Artificial Sequence Synthetic primer
caccgtggccaccggcaaggtttatgaacgttgatggccgttgtctttacgcct

SEQ ID NO: 351 - DNA, 54 bp, Artificial Sequence
  Description of Artificial Sequence Synthetic primer
caccatgaccatcaagcacaacatcatcgctgaccgctcttcattgcgtcacct

SEQ ID NO: 352 - DNA, 54 bp, Artificial Sequence
  Description of Artificial Sequence Synthetic primer
caccatgacagatgtcttgctcctgctgattccactttttcaaaccgggtggct

SEQ ID NO: 353 - DNA, 54 bp, Artificial Sequence
  Description of Artificial Sequence Synthetic primer
caccatgataagatctgtcgtacggggtaccagcgcacaagaaccgcgccccag

SEQ ID NO: 354 - DNA, 54 bp, Artificial Sequence
  Description of Artificial Sequence Synthetic primer
caccatgagtttttttcccgcacgaaccgttcgcgcatatcgccgccatgagaa

SEQ ID NO: 355 - DNA, 54 bp, Artificial Sequence
  Description of Artificial Sequence Synthetic primer
caccttgtgtaacgtcctgtcacgtaaggcttcgaggttttaccctgacgcttc

SEQ ID NO: 356 - DNA, 54 bp, Artificial Sequence
  Description of Artificial Sequence Synthetic primer
caccgtggagataggtctaacccaaattgatcggtttcgaccgcaagctctatg

SEQ ID NO: 357 - DNA, 54 bp, Artificial Sequence
  Description of Artificial Sequence Synthetic primer
caccatgaaacgcttccgcatcgttgcccgccggcgttgcggcgggtgagcgag

SEQ ID NO: 358 - DNA, 54 bp, Artificial Sequence
  Description of Artificial Sequence Synthetic primer
caccatgtcggctctacctgatcatattggttcttgtccgggttacgatattgc

SEQ ID NO: 359 - DNA, 54 bp, Artificial Sequence
  Description of Artificial Sequence Synthetic primer
caccatgatcgctgtcattttcgaagctttacatgatcgatgtcctccttatgg

SEQ ID NO: 360 - DNA, 54 bp, Artificial Sequence
  Description of Artificial Sequence Synthetic primer
caccatgaacaaagcgatccttgcagtaggaaaggagaaaactggtcgagaatc

SEQ ID NO: 361 - DNA, 54 bp, Artificial Sequence
  Description of Artificial Sequence Synthetic primer
caccttgatggagagtggcatgtccaagcggccgccagatgccggaaagtcgca

SEQ ID NO: 362 - DNA, 54 bp, Artificial Sequence
  Description of Artificial Sequence Synthetic primer
caccatggcatttgtgcatttttcgaatcggtctgcccaggtttgccgtgccgc

SEQ ID NO: 363 - DNA, 54 bp, Artificial Sequence
  Description of Artificial Sequence Synthetic primer
caccatggcaaaagcagcgaccccgaaaaggcagcttcagccgccagcttcttg

SEQ ID NO: 364 - DNA, 54 bp, Artificial Sequence
  Description of Artificial Sequence Synthetic primer
caccatggaatatttcttccagcaggtgacaccttttcgacttcaggtcggcca

SEQ ID NO: 365 - DNA, 54 bp, Artificial Sequence
  Description of Artificial Sequence Synthetic primer
caccatgaaacgcgctctgtttcttgcccatgcatatggctttcgccgtccggt

SEQ ID NO: 366 - DNA, 54 bp, Artificial Sequence
  Description of Artificial Sequence Synthetic primer
caccatggcccgctcagcccttctcaacggatcggcttcttgagtgtcttcagg

SEQ ID NO: 367 - DNA, 54 bp, Artificial Sequence
  Description of Artificial Sequence Synthetic primer
caccatgaaaatcggcatcatcaatacaattcgagcagcggcgaccaggccggg

SEQ ID NO: 368 - DNA, 54 bp, Artificial Sequence
  Description of Artificial Sequence Synthetic primer
caccatggcacctcgcgttctcgtatccgaacttccccgacgttgaactcaagc

SEQ ID NO: 369 - DNA, 54 bp, Artificial Sequence
  Description of Artificial Sequence Synthetic primer
caccatggtttctgtcccggcctcgcgccccagatccagaacaacaataccaaa

SEQ ID NO: 370 - DNA, 54 bp, Artificial Sequence
  Description of Artificial Sequence Synthetic primer
caccttggtgaagcgtatcaggaaccggaaaagcccccgtagaggctgacctga

SEQ ID NO: 371 - DNA, 54 bp, Artificial Sequence
  Description of Artificial Sequence Synthetic primer
caccatggatggttccgtggtcgtcagcctgcgcgttgcctcctgtcgtgacgg

SEQ ID NO: 372 - DNA, 54 bp, Artificial Sequence
  Description of Artificial Sequence Synthetic primer
caccatgacagatttttacagcctcatccctcggcagccggcttgaactgtgcg

SEQ ID NO: 373 - DNA, 54 bp, Artificial Sequence
  Description of Artificial Sequence Synthetic primer
caccgtggtcaatatctataactggtcggctggccggtgacgatcttggtccag

SEQ ID NO: 374 - DNA, 54 bp, Artificial Sequence
  Description of Artificial Sequence Synthetic primer
caccatgcagaacaactggtcccgcttcatcccatacggaatgctgcctgttcg

SEQ ID NO: 375 - DNA, 54 bp, Artificial Sequence
  Description of Artificial Sequence Synthetic primer
caccatgacaagtctttcctggatcacgctgtcgtggtccccggaaaggtgatg

SEQ ID NO: 376 - DNA, 54 bp, Artificial Sequence
  Description of Artificial Sequence Synthetic primer
caccatgaacaggcaccgcaaagggttcacgatttgcgcccagtgccatcaaca

SEQ ID NO: 377 - DNA, 54 bp, Artificial Sequence
  Description of Artificial Sequence Synthetic primer
caccatgaagtgttatcttattaatctcgcgcaatatcccgatcaaatggaatg

SEQ ID NO: 378 - DNA, 54 bp, Artificial Sequence
  Description of Artificial Sequence Synthetic primer
caccatgcgggggcaggcaattgcgataaggccgtcgaccataccgtgtgcacg

SEQ ID NO: 379 - DNA, 54 bp, Artificial Sequence
  Description of Artificial Sequence Synthetic primer
caccatggatcaccacgcgggcattcccgatcctttgttttgacgcgcatcttt

SEQ ID NO: 380 - DNA, 54 bp, Artificial Sequence
  Description of Artificial Sequence Synthetic primer
caccatgtccgtatccaagaaatatatcctttatccggcgtggaggcagtatcg

SEQ ID NO: 381 - DNA, 54 bp, Artificial Sequence
  Description of Artificial Sequence Synthetic primer
caccatggtgacggccagaatgcgcgatgaacgtagagcctttcataaagtccg

SEQ ID NO: 382 - DNA, 54 bp, Artificial Sequence
  Description of Artificial Sequence Synthetic primer
caccatgaacaatgctctcctcgattccagaagaacaccttgaagcgttcagcg

SEQ ID NO: 383 - DNA, 54 bp, Artificial Sequence
  Description of Artificial Sequence Synthetic primer
caccatggccgcggccatgcttctgcccgacgcacccagaatgagcgtgtgaag

SEQ ID NO: 384 - DNA, 54 bp, Artificial Sequence
  Description of Artificial Sequence Synthetic primer
caccatgcttcgccaggagatttcccgggttgcagcagcgattttacaagtgcg

SEQ ID NO: 385 - DNA, 54 bp, Artificial Sequence
  Description of Artificial Sequence Synthetic primer
caccttgcagtccgtcctgcgggagggagggccaaatgcggcgcagaagcaaac

SEQ ID NO: 386 - DNA, 54 bp, Artificial Sequence
  Description of Artificial Sequence Synthetic primer
caccatgccgaaaacgacggggggtggcgtgagatccgtcctgtttttttcttg

SEQ ID NO: 387 - DNA, 54 bp, Artificial Sequence
  Description of Artificial Sequence Synthetic primer
caccatggcgtcaatcgaaatcttgatcattttccccgaaaacctgtcttctgc

SEQ ID NO: 388 - DNA, 54 bp, Artificial Sequence
  Description of Artificial Sequence Synthetic primer
caccatgaccggcgcagaaatggttatccgacgagcgcgcggcccttggcatca

SEQ ID NO: 389 - DNA, 54 bp, Artificial Sequence
  Description of Artificial Sequence Synthetic primer
caccatgaacgcacaaaacctagcttccgcatgccttccgcaccacggttcatg

SEQ ID NO: 390 - DNA, 54 bp, Artificial Sequence
  Description of Artificial Sequence Synthetic primer
caccatgctttacggtattttacttgcgtgaaataccccgctttttcttggacc

SEQ ID NO: 391 - DNA, 54 bp, Artificial Sequence
  Description of Artificial Sequence Synthetic primer
caccatgagcgaacccaattccgaaacgaacgctcgcccgtggcgcgcttgcgg

SEQ ID NO: 392 - DNA, 54 bp, Artificial Sequence
  Description of Artificial Sequence Synthetic primer
caccatgacgggttccggaaaccgcctaattccgtgccttgaatagcggcgagt

SEQ ID NO: 393 - DNA, 54 bp, Artificial Sequence
  Description of Artificial Sequence Synthetic primer
caccatgaaaaagctccttgcacattccgatccaccaccgaaacgctcaccgtg

SEQ ID NO: 394 - DNA, 54 bp, Artificial Sequence
  Description of Artificial Sequence Synthetic primer
caccgtgatcggattaaaatgcttgagtgcgacgtgatcgtcagcccggtcatc

SEQ ID NO: 395 - DNA, 54 bp, Artificial Sequence
  Description of Artificial Sequence Synthetic primer
caccatgctctattgcgaaacatccaatgtccacgaacttctgcaatctgcacg

SEQ ID NO: 396 - DNA, 54 bp, Artificial Sequence
  Description of Artificial Sequence Synthetic primer
caccgtgcgcgaatattatacgataacggggtcgatacgccaagctccgccaga

SEQ ID NO: 397 - DNA, 54 bp, Artificial Sequence
  Description of Artificial Sequence Synthetic primer
caccatgtcgaaaccggttattacccgttaccgatttgatcgccgatgcgggcg

SEQ ID NO: 398 - DNA, 54 bp, Artificial Sequence
  Description of Artificial Sequence Synthetic primer
caccatgatcgacgatatctacaataaacggcggctttcgccttttcctcgatc

SEQ ID NO: 399 - DNA, 54 bp, Artificial Sequence
  Description of Artificial Sequence Synthetic primer
caccatggggtcttgcggcgggaaacatattttccggtgcgcgaagccgagcag

SEQ ID NO: 400 - DNA, 54 bp, Artificial Sequence
  Description of Artificial Sequence Synthetic primer
caccatgaaatcatcccgcaatattgatccaggccttttttcttcgcttcgctc

SEQ ID NO: 401 - DNA, 54 bp, Artificial Sequence
  Description of Artificial Sequence Synthetic primer
caccgtgagcactccgccgaaaccattgcttccaaccaccagcccgcatccgtg

SEQ ID NO: 402 - DNA, 54 bp, Artificial Sequence
  Description of Artificial Sequence Synthetic primer
caccatgtcgtcagtcctttcccgctatgattccaggccgggatgccggtgccc

SEQ ID NO: 403 - DNA, 54 bp, Artificial Sequence
  Description of Artificial Sequence Synthetic primer
caccatgacgccggagactgcattgatcatgctgcctgtttgtgcgctgcgctg

SEQ ID NO: 404 - DNA, 54 bp, Artificial Sequence
  Description of Artificial Sequence Synthetic primer
caccatgaaaaatgcggcttccattacgacttctttgcagcgtcccgggcggcc

SEQ ID NO: 405 - DNA, 54 bp, Artificial Sequence
  Description of Artificial Sequence Synthetic primer
caccatgtttcaaaagatactcatagccaggcgctttcgttcttgctcttgttg

SEQ ID NO: 406 - DNA, 54 bp, Artificial Sequence
  Description of Artificial Sequence Synthetic primer
caccttgggttttttcctgaaaagccttgaataaagccggaataattcctaagc

SEQ ID NO: 407 - DNA, 54 bp, Artificial Sequence
  Description of Artificial Sequence Synthetic primer
caccatgaaaaatctgcattccttgtttctttcgacaggcagtctttcagcgaa

SEQ ID NO: 408 - DNA, 54 bp, Artificial Sequence
  Description of Artificial Sequence Synthetic primer
caccatgtttcgatggggtgtactttccacacagtctcgctcaccccgcttctg

SEQ ID NO: 409 - DNA, 54 bp, Artificial Sequence
  Description of Artificial Sequence Synthetic primer
caccatgaagttcacgagaacgctggtgcgaacttgaaggccgtctggaaagtg

SEQ ID NO: 410 - DNA, 54 bp, Artificial Sequence
  Description of Artificial Sequence Synthetic primer
caccatgagcgagatcggttcttatcacgcttcccgatgcagaattgcgagaaa

SEQ ID NO: 411 - protein, 6 aa, Artificial Sequence
  Description of Artificial Sequence Synthetic 6xHis tag
HHHHHH

SEQ ID NO: 412 - DNA, 20 bp, Artificial Sequence
  Description of Artificial Sequence Synthetic primer
taatacgactcactataggg

SEQ ID NO: 413 - DNA, 20 bp, Artificial Sequence
  Description of Artificial Sequence Synthetic primer
tagttattgctcagcggtgg